(19) United States
(12) Patent Application Publication
(10) Pub. No.: US 2006/0094100 A1
(43) Pub. Date: May 4, 2006

(54) Structural models of carnitine acyltransferases and uses thereof

(76) Inventors: Liang Tong, Scarsdale, NY (US); Gerwald Jogl, New York, NY (US)

Correspondence Address: BAKER & BOTTS, 30 ROCKEFELLER PLAZA, NEW YORK, NY, 10112, US

(21) Appl. No.: 11/146,700

(22) Filed: Jun. 7, 2005

Related U.S. Application Data
(63) Continuation of application No. PCT/US04/00170, filed on Jan. 6, 2004.
(60) Provisional application No. 60/438,172, filed on Jan. 6, 2003.

(30) Foreign Application Priority Data
Jul. 29, 2004 (WO) 2004/062581

(51) Int. Cl. G06F 19/00; C12N 9/10

(52) U.S. Cl. 435/193; 702/19

(57) ABSTRACT

The present invention relates to structural models of carnitine acyltransferases, and, in particular, to models of the reactive sites of these enzymes. It is based, at least in part, on the X-ray crystallographic structures of murine carnitine acetyltransferase (“mCRAT”), both in pure form and in complex with its substrates carnitine and coenzyme A (“CoA”). The structural information provides a basis for designing modulators of the activity of CRAT and related enzymes.

### CROSS-REFERENCE TO RELATED APPLICATION

The present application claims priority from U.S. Patent Application No. 60/438,172, filed Jan. 6, 2003, the entire disclosure of which is incorporated herein by reference.

### INTRODUCTION

The present invention relates to structural models of carnitine acyltransferases, and, in particular, to models of the reactive sites of these enzymes. It is based, at least in part, on the X-ray crystallographic structures of murine carnitine acetyltransferase (“mCRAT”), both in pure form and in complex with its substrates carnitine and coenzyme A (“CoA”). The structural information provides a basis for designing modulators of the activity of CRAT and related enzymes.

### BACKGROUND OF THE INVENTION

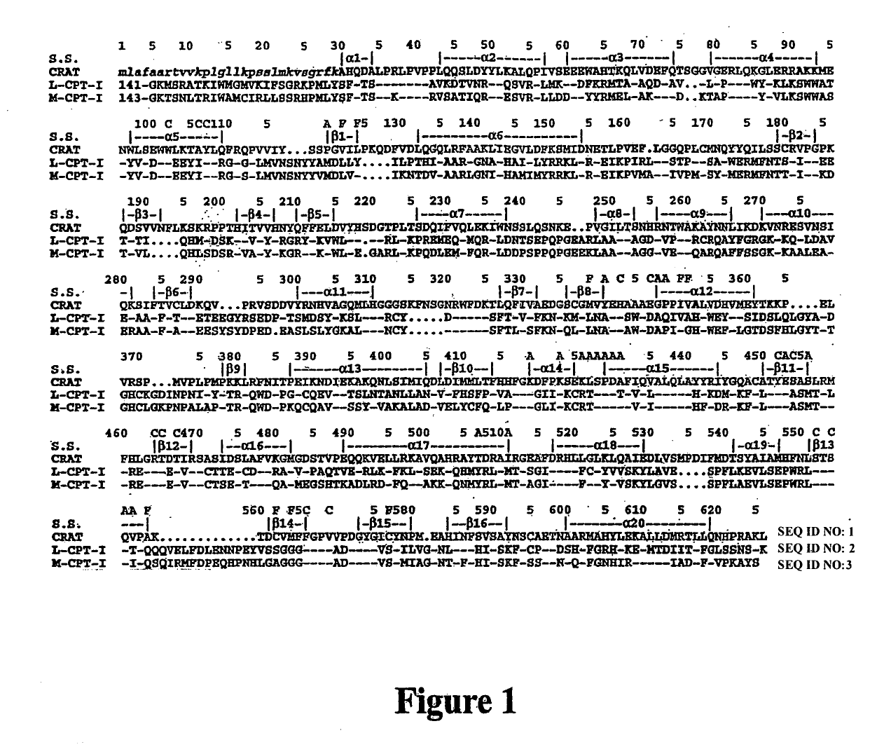

Carnitine acyltransferases are a group of structurally related enzymes involved in lipid catabolism. More specifically, these enzymes participate in fatty acid oxidation, catalyzing the exchange of acyl groups between carnitine and CoA (Bieber, 1988, Ann. Rev. Biochem. 57:261-283; Kerner and Hoppel 2000, Biochim. Biophys. Acta 1486:1-17; McGarry and Brown, 1997, Eur. J. Biochem. 244:1-14; Ramsay et al., 2001, Biochim. Biophys. Acta 1546:21-43). Among the carnitine acyltransferases are carnitine acetyltransferase (CRAT, also known as CAT), carnitine octanoyltransferase (COT) and carnitine palmitoyltransferase (CPT), with substrate preferences for short-chain, medium-chain and long-chain fatty acids, respectively. These enzymes generally contain approximately 600 amino acid residues and have molecular weights of about 70 kD (FIG. 1); they are the products of a multi-gene family which may have evolved by duplication of a single ancestral gene (van der Leij et al., 2000, Mol. Genet. Metab. 71(1-2):139-153).

The fatty acid oxidation process begins when a fatty acid is activated in the cell cytoplasm to form acyl-CoA. The acyl moiety is then transported into the mitochondria via an acyl-carnitine intermediate formed by CPT-I in the outer mitochnodrial membrane and regenerated into acyt-CoA and carnitine by CPT-If in the inner mitochondrial membrane (McGarry and Brown, 1997, Eur. J. Biochem. 244:1-14; Ramsay et al., 2001, Biochim. Biophys. Acta 1546:21-43). Once in the mitochondrial matrix, the acyl group is broken down by a cyclic pathway that shortens the carbon backbone by 2-carbon segments per round and indirectly generates molecules of the important cellular energy source, adenosine triphosphate (ATP). The residual 2-carbon fragments are in the form of acetyl-CoA. Acetyl-CoA can generate further energy by entering the Krebs cycle during aerobic respiration.

CRAT catalyzes the reversible conversion of acetyl-CoA and carnitine to acetyl-carnitine and CoA, and thereby regulates the balance between acetyl-CoA and free CoA (Liu et al., 2002, Proc. Natl. Acad. Sci. U.S.A. 99(4):1876-1881). The cellular compartmentalization of acetyl-CoA is apportioned such that a majority of acetyl-CoA resides in mitochondria and a smaller pool is maintained in the cytosol (Lo Giudice et al., 2002, Diabetes Res. Clin. Pract. 56(3):173-180).

To efficiently control energy expenditure, the relative rates of fatty acid synthesis and oxidation are cross-regulated, and conditions that favor fatty acid synthesis disfavor lipid oxidation. For example, malonyl-CoA, an intermediate in fatty acid synthesis, inhibits the activity of the catabolic enzyme CPT-I (Abu-Elheiga et al., 2001, Science 291:2613-2616; Morillas et al., 2002, J. Biol. Chem. 277(13): 11473-11480). Thus, when it is appropriate for a normal, healthy cell to store energy (by synthesizing fatty acids), the oxidation of fatty acids is inhibited. Conversely, when the cell is expending energy, the consequent low levels of ATP are associated with inhibition of malonyl-CoA production (and hence fatty acid synthesis), which “disinhibits” CPT-I and favors fatty acid oxidation.

The significant role played by CRAT can be appreciated by considering the importance of one of its substrates, acetyl-CoA, as an energy resource. As discussed above, acetyl-CoA is the end product of fatty acid oxidation. In addition, acetyl-CoA can be produced by catabolism of carbohydrates. In aerobic respiration, pyruvate, generated from glucose via the Embden-Meyerhoff pathway (which is shared by aerobic and anaerobic respiration) is oxidized by pyruvate dehydrogenase and combined with CoA to form acetyl-CoA and carbon dioxide. However, when oxygen supplies are low, pyruvate is instead reduced to form lactic acid, thereby regenerating a component (NAD+) needed to release additional energy from glucose stores by (anaerobic) glycolysis. For oxygen-deprived cells, the need to exploit all possible energy sources counterbalances the toxic effects (e.g. muscle fatigue and cramps) of accumulating lactic acid.

The activity of pyruvate dehydrogenase (“PDH”), and hence the ability of a cell to choose whether to utilize carbohydrates in aerobic respiration, is affected by the acetyl-CoA/CoA ratio, an equilibrium partially regulated by CRAT (Bieber, 1988, Ann. Rev. Biochem. 57:261-283; Ramsay et al., 2001, Biochim Biophys Acta 1546:21-43; van der Leij et al., 2000, Mol. Genet. Metab. 71(1-2):139-153). Acetyl-CoA competes with CoA for binding to PDH enzyme and activates a kinase that inhibits PDH. If the reaction to produce acetyl-CoA+ carnitine is favored, increasing the acetyl-CoA/CoA ratio, PDH is inhibited and glucose-derived pyruvate is blocked from entering the Krebs cycle (moreover, carnitine is freed to shuttle fatty acids into the mitochondria for oxidation).

An organism's physiologic choice to utilize carbohydrate versus fat to supply energy can have either positive or negative effects. For example, during starvation, if muscle cells are allowed to deplete carbohydrate reserves, there may not be sufficient glucose available for neurons in the brain to survive; it is therefore advantageous for the cells to utilize fat as their energy source. Certain diets are designed to influence the body's choice to preferentially tap fat reserves (Forslund et al., 1999, Am. J. Physiol. 276:964-976). On the detrimental side, high levels of free fatty acids in obese individuals are postulated to induce insulin resistance (suppressing glucose utilization), thereby providing a link between obesity and diabetes (Boden, 1997, Diabetes 46:3-10).

Aberrancies in utilization of glucose versus fat are believed to play a significant role in diabetes in non-obese as well as in obese subjects. As reviewed in Anderson, 1998, Cur. Pharmaceut. Des. 4:1-15, adipocytes, major sites of fatty acid storage, are subject to insulin resistance in diabetic patients, so that lipase activity, which normally would be inhibited by insulin, increases. The consequent rise in levels of free fatty acids (“FFA”) leads to increased fatty acid oxidation, decreased glucose oxidation and increased utilization of lactate to re-form glucose. Anderson states that “[t] aken together, elevated FFA increase hepatic glucose production, decrease peripheral gluocse disposal and impair pancreatic insulin secretion, all hallmarks of the diabetic condition.”

In addition to its role in maintaining the acetyl-CoA/CoA equilibrium, CRAT's physiologic importance is demonstrated by data suggesting that CRAT activity may be required for progression through G1 to the S phase of the cell cycle (Brunner et al., Biochem J. 322:403-420). Moreover, inherited defects in CRAT have been associated with severe neurological disorders known as encephalopathies (DiDonato et al., 1979, Neurology 29(12):1578-1583; Melegh et al., 1999, J. Inherit. Metab. Dis. 22(7):827-838). CRAT activity has been observed to decrease with age in rat soleus, diaphragm, and heart muscles (Liu et al., 2002, Proc. Natl. Acad. Sci. U.S.A. 99(4):1876-1881 (“Liu et al.”), citing Hansford, 1978, Biochem. J. U178:285-295 and Hansford and Castro, 1982, Mech. Aging Dev. 19:191-200), in brain and muscles of vitamin E-deficient rats (Liu et al., citing Sung et al., 1978, Neurochem. Res. 3:815-820), in the microvessels and cerebellum of the brains of Alzheimer's Disease patients (Liu et al., citing Kalaria and Harik, 1992, Annal. Neurol. 32:583-586 and Makar et al., 1995, Neurochem. Res. 20:705-711, with contradictory findings reported in Maurer et al., 1998, Alzheimer Dis. Assoc. Disord. 12:71-76), and in severe peripheral vascular disease (Liu et al., citing Brevetti et al., 1991, Circulation 84:1490-1495).

Likewise, mutations in other members of the carnitine acyltransferase family have been associated with various pathologies. Inherited recessive defects of CPT-1 and CPT-II can give rise to hypoketonemia and hypoglycemia, with severely reduced blood glucose levels (McGarry and Brown, 1997, Eur. J. Biochem. 244:1-14, Ramsay et al., 2001, Biochim. Biophys. Acta 1546:21-43). CPT-II deficiency is the most common cause of abnormal lipid metabolism in skeletal muscle, causing muscle pain and myoglobinuria (DiMauro and Melis-DiMauro, 1973, Science 182:929-931). Both single-point mutations and insertions/deletions in these genes have been observed to produce the clinical phenotype.

The physiologic relevance of carnitine acyltransferases not only is a source of pathology when these enzymes go awry, but also provides opportunities for treatment of diseases linked with disorders in fatty acid metabolism. As discussed above, the hyperglycemia found in diabetes results from decreased glucose disposal and increased glucose production, associated with increased and uncontrolled fatty acid oxidation (Bebernitz and Schuster, 2002, Curr. Pharm. Des. 8(14):1199-1227). Inhibition of fatty acid oxidation has emerged as a new strategy for the treatment of diabetes (Id.; Wagman and Nuss, 2001, Curr. Pharm. Des. 7(6):417-450), in particular non-insulin dependent diabetes mellitus (“NIDDM”; also known as “mature onset diabetes”).

Agents which directly inhibit oxidation of fatty acids have to date been unsuccessful for NIDDM treatment because of severe side effects, most notably hypoglycemia, but also hypoketosis, hyperammonemia, aciduria. hypotonia, liver failure and cardiomyopathy (Anderson, 1998, Curr. Pharmaceut. Des. 4:1-15). Agents targeted at fatty acid transport, such as CPT inhibitors, provide more promising options. In particular, a covalent inhibitor of L-CPT-I, etomoxir, has been evaluated for its therapeutic benefit in NIDDM. While early reports indicated that etomoxir reduced hepatic glucose production and plasma lipids in NIDDM patients (Ratheiser et al., 1991, Metabolism 40(11):1185-1190) and was said to improve insulin sensitivity (Hubinger, 1992, Horm. Metab. Res. 24(3):115-118), it was later found to be associated with cardiac toxicities that seemed not to be counterbalanced by its effectiveness. Furthermore, it was demonstrated that high doses of etomoxir resulted in insulin resistance in rats (Dobbins et al., 2001, Diabetes 50: 123-130). Surprisingly, etomoxir was later tested for use in the treatment of certain heart diseases (Hayashi et al., 2001, Life Sci. 68(13):1515-1526; Zarain-Herzberg and Rupp, 2002, Expert Opin. Investig. Drugs 11(3):345-356; Kato et al., 2002, Mol. Cell. Biochem. 232(1-2):57-62), and has been considered as an anti-obesity agent (Hinderling et al., 2002, Am. J. Clin. Nutr. 76(1):141-147).

There have been favorable reports regarding treatment of diabetes and other disorders with carnitine or acetylcarnitine, both of which are substrates for CRAT. Carnitine deficiency has been associated with complications (e.g., retinopathy, hyperlipidemia, and neuropathy) of diabetes (Tamamogullari et al., 1999, J. Diabetes Complic. 13(5-6):251-253), and intravenous carnitine has been found to stimulate glucose disposal in normal individuals (De Gaetano et al., 1999, J. Am. Coll. Nutr. 8(4):289-295) and diabetic patients (Mingrone et al., 1999, J. Am. Coll. Nutr. 18(1):77-82). Acute carnitine supplementation has also been reported to improve the recovery of ischemic myocardium in diabetic as well as euglycemic rats (Keller et al., 1998, Ann. Thorac. Surg. 66(5):1600-1603).

Paradoxically, acetylcarnitine has also been found to increase glucose disposal in diabetic patients (Giancaterini et al., 2000, Metabolism 49(6):704-708) and to prevent autonomic neuropathy in diabetic rats (Lo Giudice et al., 2002, Diabetes Res. Clin. Pract. 56(3:173-180). In addition, acetylcarnitine has been observed to retard brain mitochondrial decay in aged rats (Liu et al., 2002, Proc. Natl. Acad. Sci. U.S.A. 99(4):1876-1881; Liu et al., 2002, Ann. N.Y. Acad. Sci. 959:133-166). To explain the ability of acetylcarnitine (“ALC”) to increase glucose disposal, Lo Guidice propose that “ALC infusion very likely produces an increase in the acetyl-CoA cytosolic pool and simultaneously increases the ALC cellular concentration, forcing the passage of acetyl moieties across the mitochondrial membrane.” As a consequence, “a back-inhibition of [fatty acid] oxidation would occur with a subsequent reduction of acetyl-CoA formation.” The decreased levels of acetyl-CoA would favor PDH activity to funnel glucose stores into the Krebs cycle.

Although positive effects have been associated with carnitine and acetylcarnitine, neither compound has been accepted by the medical community as a suitably effective therapeutic agent for the management of diabetes. Despite the various incentives for developing other modulators of carnitine acyltransferases, including CRAT, rational drug design of such compounds has been problematic because, prior to the present invention, structural information available for these enzymes has been extremely limited. In the early 1980's, methoxycarbonyl-CoA disulfide was used as an active-site-directed inhibitor of carnitine acetyltransferase, and led to the hypothesis that CRAT has a sulfhydryl group at the site of acetyl group transfer (Venkatraghavan and Smith, 1983, Arch. Biochem. Biophys. 220(1):193-199. However, as shown by the present invention, there is no cysteine residue in the active site and therefore, no sulfhydryl group. Later inhibitor-based studies of the CRAT active site used metabolites of 4-pentenoic acid (Zhong et al., 1985, Arch. Biochem. Biophys. 240:524-529), hemiacetylcarnitinium chloride (Gandour et al., 1986, Biochem. Biophys. Res. Commun. 138(2):735-741), and acetyl-DL-aminocarnitine (Brass et al., 1991, Biochim. Biophys. Acta 1095(1):17-22). Circular dichroism and fluorescence spectroscopy were used to study interactions between CRAT and its substrates (Yan et al., 1995, Biochim. Biophys. Acta 1253(2):175-180). Lian et al. (2002, Acta Cryst D58:1193-1194) report preliminary X-ray crystallographic studies of recombinant human carnitine acetyltransferase; a photographic image is provided but no atomic coordinates are given. None of these studies, taken singly or in combination, provide a sufficiently detailed structure to elucidate the catalytic mechanisms of carnitine acyltransferases. A further impediment to rational drug design of modulators of carnitine acyltransferases is the lack of recognizable sequence homology between this family of enzymes and other proteins in public protein sequence databases. Structural based models for these enzymes have been published (Morillas et al., 2001, J. Biol. Chem. 276:45001-45008; Morillas et al., 2002, J. Biol. Chem. 277:11473-11480) which are inaccurate, demonstrating the need for genuine, experimentally-determined structural information.

To understand the molecular mechanism for catalysis by this important family of enzymes and to provide a structural basis for the design and/or identification of modulator compounds, the crystal structures of mouse CRAT, alone and in complex with its substrates carnitine or CoA, at up to 1.8 Å resolution have been determined. These crystal structures are the bases for models of the murine CRAT substrate binding sites and active site. From the murine structures, models of human CRAT, its substrate binding sites and active site have been derived. As the amino acid sequences among the various carnitine acyltransferases are significantly conserved among the eukaryotes (FIG. 1), the structural observations shown by the present invention for CRAT can be generally applied to this entire family of enzymes.

### SUMMARY OF THE INVENTION

The present invention relates to structural models of carnitine acyltransferases, and, in particular, to models of the reactive sites of these enzymes. It is based, at least in part, on the X-ray crystallographic structure of murine CRAT, alone and bound to substrate, and on the structure of human CRAT deduced therefrom.

The structural information of the present invention is useful, inter alia, for the rational design of compounds which act as modulators of carnitine acyltransferase activity. Furthermore, the invention provides for actual or virtual screening assays which may be used to identify compounds (for example, members of a combinatorial library) having such modulator activities, or to confirm the activity of designed compounds. In view of the biological importance of carnitine acyltransferases and carnitine acetyltransferase (“CRAT”) in particular, modulators of the activities of these enzymes may be used in the treatment of various diseases and disorders, including, but not limited to, non-insulin dependent diabetes mellitus (“NIDDM”) and its complications, obesity, starvation, heart disease (ischemic or non-ischemic) and disorders associated with mitochondrial dysfunction and/or aging (including, but not limited to, inherited mutations in carnitine acyltransferases and Alzheimer's disease). The present invention may also be used to design and/or screen metabolic enhancers that may be used to promote endurance or survival under stressful conditions. Additionally, modulators having toxic effects may be used as chemotherapeutic agents in the treatment of cancer or other proliferative diseases, as pesticides, or as herbicides, to name but a few uses.

In one embodiment, the present invention provides the atomic coordinates which define the three-dimensional structure of CRAT alone or in complex with its substrates, carnitine and CoA, with a root mean square deviation (RMSD) of from about 0 to 4 Å along the Ca chain of CRAT. Preferably the RMSD is from about 0 to 2 Å. In another embodiment, the RMSD is from about 0 to 0.5 Å. In another embodiment of the present invention, there is provided the atomic coordinates which define the three-dimensional structure of the substrate binding sites of CRAT alone and in complex with carnitine or CoA, with a root mean square deviation (RMSD) of from about 0 to 4 Å along the Ca chain of the substrate binding site. Preferably the RMSD is from about 0 to 2 Å. In another embodiment, the RMSD is from about 0 to 0.5 Å. In an additional, related embodiment, the present invention provides a structure for the reactive site of CRAT. The atomic coordinates may be included in a computer readable medium, including a database, and may be displayed on a computer display.

In further embodiments, the present invention provides for methods of using a computer to identify modulators of a target carnitine acyltransferase comprising using a computer-readable three-dimensional structure of a CRAT enzyme, a CRAT substrate binding site, and/or a CRAT reactive site to design and/or select a potential modulator of the carnitine acyltransferase based on the predicted ability of the modulator to bind to said CRAT enzyme, CRAT substrate binding site and/or CRAT reactive site. The invention further provides for synthesizing and then testing the designed or selected modulator for its ability to modulate the activity of the target carnitine acyltransferase. For example, a potential modulator may be contacted with the target enzyme in the presence of one or more substrate, and the ability of the target enzyme to act on its substrate in the presence or absence of potential modulator may be measured and compared. As another specific, non-limiting example, a designed or selected potential modulator may be synthesized and introduced into an in vivo or in vitro model system for fatty acid oxidation, and the effect of the modulator on the acetyl-CoA/CoA ratio may be determined. A modulator that decreases the relative amount of acetyl-CoA may have the therapeutic benefit of favoring the utilization of glucose as an energy source (as would be useful in the treatment of a diabetic subject).

The present invention also provides for molecules which comprise a carnitine acyltransferase substrate binding and/or active site, as defined by the structures for the murine and human CRAT defined herein, in an otherwise synthetic molecule. Such a molecule could be used to screen test compounds, for example compounds in a combinatorial library, for binding to the active site and/or for suitability as substrates.

In still further embodiments the present invention provides for a method of designing or selecting an inhibitor or agonist of a carnitine acyltransferase comprising creating a computer model of the negative space present in an unoccupied CRAT substrate binding site or active site, preferably taking into account the electron densities at the boundaries of this space, and using such a model to design or select molecules that modulate the activity of CRAT or another carnitine acyltransferase enzyme. Such a negative space, particularly a space presented in the context of electrophilic and electrophobic boundaries, in computer readable, electronic form, stored or storable on a floppy disk or computer hard drive, may provide a simple template for the design/or and selection of enzyme-interactive compounds.

In addition, the present invention provides for a method of evaluating the binding properties of a potential modulator comprising co-crystallizing the potential modulator with CRAT, determining the three-dimensional structure of the modulator bound to CRAT and analyzing the three-dimensional structure of CRAT bound to the modulator to evaluate the structural aspects of binding.

### DEFINITIONS

The term “α helix” refers to the most abundant helical conformation found in globular proteins. In an α helix, all amide protons point toward the N-terminus and all carbonyl oxygens point toward the C-terminus. The repeating nature of the phi, psi pairs ensure this orientation. Hydrogen bonds within an a helix also display a repeating pattern in which the backbone C═O of residue X (wherein X refers to any amino acid) hydrogen bonds to the backbone HN of residue X+4. The ÿ helix is a coiled structure characterized by 3.6 residues per turn, and translating along its axis 1.5 Å per amino acid. Thus the pitch is 3.6×1.5 or 5.4 Å. The screw sense of alpha helices is always right-handed.

The term “active site” is a region of the carnitine acyltransferase which contains the amino acid residues necessary for acyl transfer. For mCRAT, the amino acids that may participate in acetyl transfer are TRP102, LEU103, ALA106, TYR107, ILE116, TYR117, SER118, SER119, PRO120, GLY121, TYR341, GLU342, HIS343, ALA344, ALA345, ALA346, GLU347, GLY348, PRO349, PRO350, ILE351, TYR452, GLU453, SER454, ALA455, SER456, LEU457, ARG458, ARG464, THR465, ASP466, THR467, IL468, ARG469, ARG518, LEU551, SER552, THR553, SER554, GLN555, VAL556, MET564, PHE565, PHE566, GLY567, PRO568, and VAL569 and preferably HIS 343, and for hCRAT the amino acids that may participate in acetyl transfer are TRP102, LEU103, ALA106, TYR107, ILE116, TYR117, SER118, SER119, PRO120, GLY121, TYR341, GLU342, HIS343, ALA344, ALA345, ALA346, GLU347, GLY348, PHE349, PRO350, ILE351, TYR452, GLU453, SER454, ALA455, SER456, LEU457, ARG458, ARG464, THR465, ASP466, THR467, ILE468, ARG469, ARG518, LEU551, SER552, THR553, SER554, GLN555, VAL556, MET564, PHE565, PHE566, GLY567, PRO568, and VAL569, and preferably HIS343.

The term “β sheet” refers to two or more polypeptide chains (or β strands) that run alongside each other and are linked in a regular manner by hydrogen bonds between the main chain C═O and N—H groups. Therefore all hydrogen bonds in a beta-sheet are between different segments of polypeptide. Hydrogen bonds in antiparallel sheets are perpendicular to the chain direction and spaced evenly as pairs between strands. Hydrogen bonds in parallel sheets are slanted with respect to the chain direction and spaced evenly between strands.

The term “carnitine acyltransferase”, as used herein, refers to a structurally and functionally related family of enzymes that includes carnitine acetyltransferase (“CRAT”), carnitineoctanoyltransferase (“COT”), carnitine palmitoyltransferase (“CPT”; including LCPT-I and M-CPT-II), and allelic variants and mutations thereof that are at least 85 percent, preferably at least 90 percent, and more preferably at least 95 percent homologous to the naturally occurring enzyme, with homology being determined by standard computer software, such as BLAST P, or ClustalW used with a scoring matrix such as BLOSUM or PAM.

The term “CRAT” refers to the genus of CRAT enzymes, irrespective of their organismal species of origin; it encompasses mammalian CRATs (“mamCRATs”) such as murine (“mCRAT”) and human (“hCRAT”) carnitine acetyltransferases, and allelic variants and mutations thereof that are at least 85 percent, preferably at least 90 percent, and more preferably at least 95 percent homologous to the naturally occurring enzyme, with homology being determined by standard computer software, such as BLASTP, or ClustalW used with a scoring matrix such as BLOSUIM or PAM. The nucleic acid sequence of a cDNA encoding mCRAT and the amino acid sequence of mCRAT protein is publicly available as GenBank Accession No. NP_{—}031786 (Brunner et al., 1997, Biochem. J. 322(2):403-410). The nucleic acid sequence of a cDNA encoding hCRAT and the amino acid sequence of hCRAT protein is publicly available as GenBank Accession No. CAA55359 (Corti et al., 1994, Genomics 23(1):94-44).

The term “loop” refers to any other conformation of amino acids. (i.e. not a helix, strand or sheet). Additionally, a loop may contain bond interactions between amino acid side chains, but not in a repetitive, regular fashion.

A “modulator” of enzyme activity as used herein refers to a compound which can alter the amount of product generated by a reaction catalyzed by the enzyme. The alteration may be an increase or a decrease. A compound that increases the amount of product is considered an agonist and a compound that decreases the amount of product is considered an inhibitor. Where the biological function of an enzyme encompasses both directions of a reaction (for example CRAT catalyzes the reaction of acetyl-CoA+carnitine<−>acetylcarnitine+CoA in both directions, with the directionality controlled by cellular factors such as substrate concentration), whether a modulator is acting as an agonist or an inhibitor depends upon the product being considered. Therefore, the term “modulator” is used in the alternative for product-specific agonist or antagonist activity. For example, if a compound promotes CRAT-mediated acetyl-CoA+carnitine−>acetylcarnitine+CoA, it may either be referred to as a CRAT modulator that favors this direction of the reaction, or it may be referred to as a CRAT agonist of acetylcarnitine and CoA formation or a CRAT antagonist of acetyl-CoA and carnitine formation.

The term “reactive site” refers to a region of an enzyme, a portion of an enzyme, or an actual or virtual model thereof, which provides an active site and one or more substrate binding sites. It is possible that, in some instances, an active site and a substrate binding site overlap or coincide.

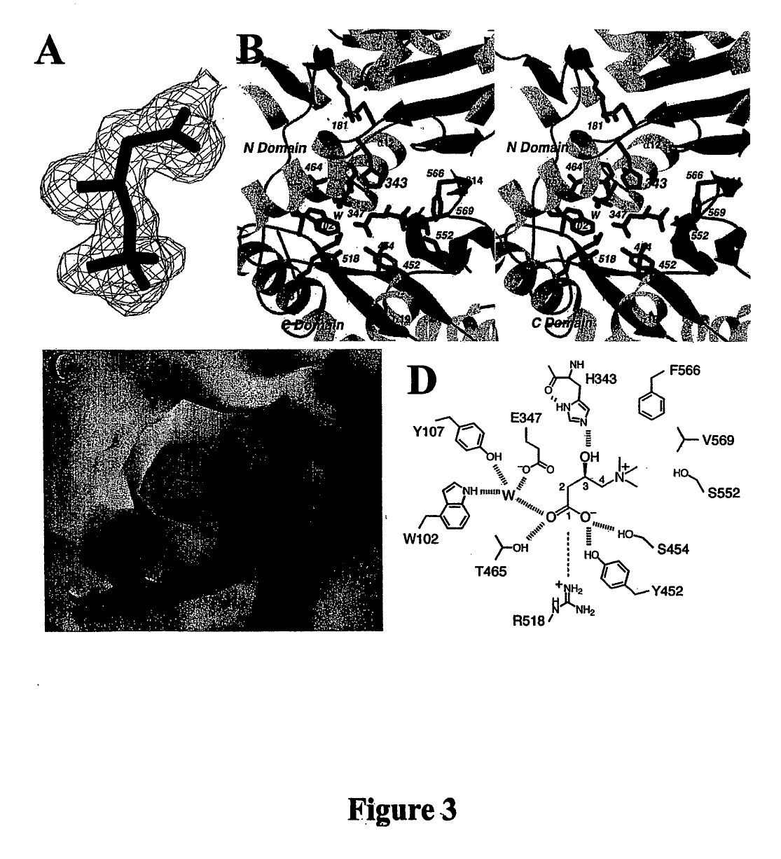

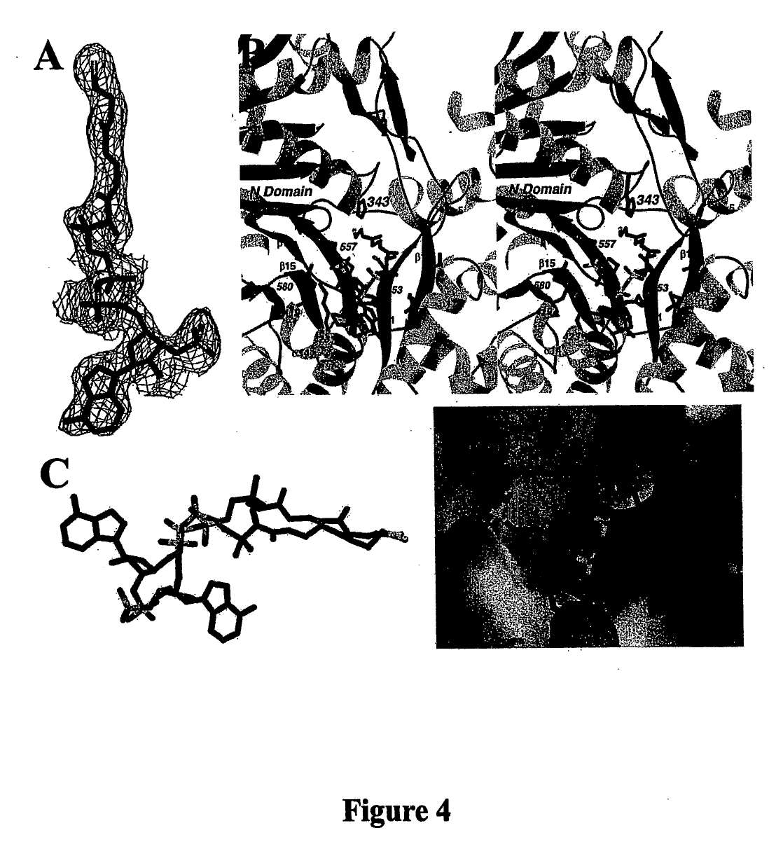

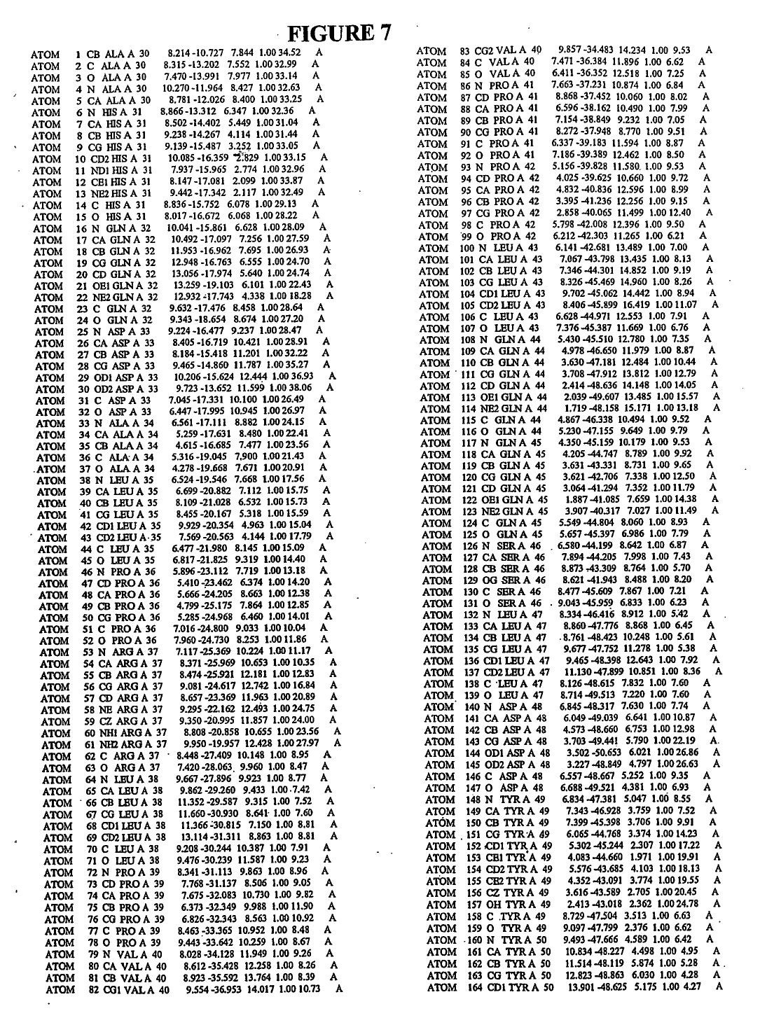

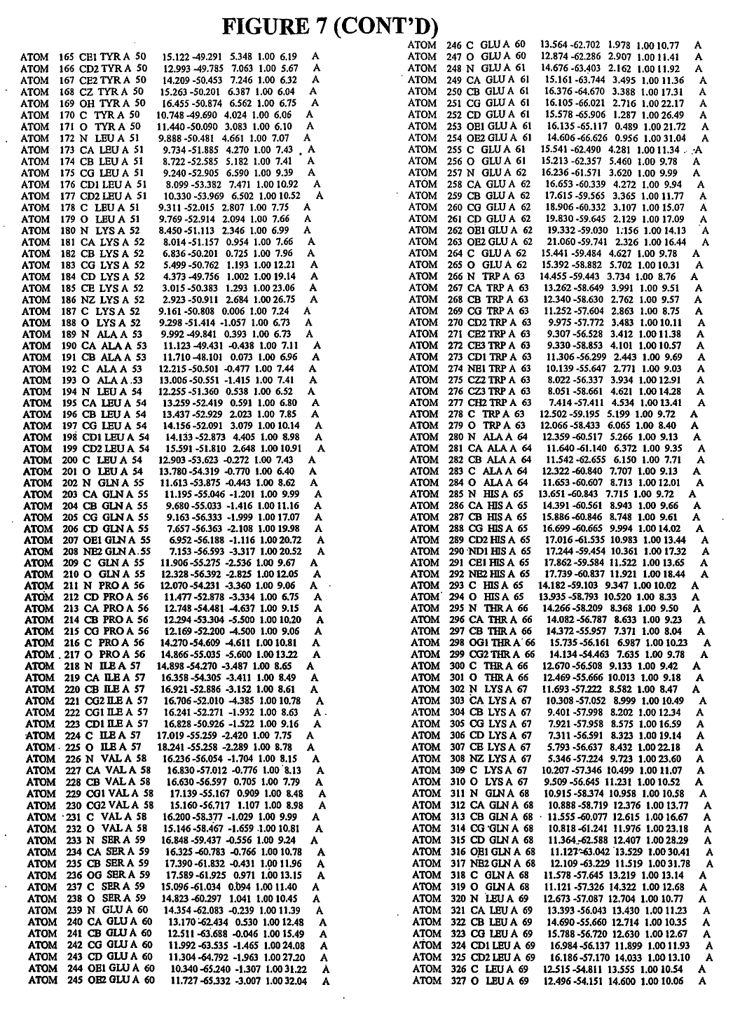

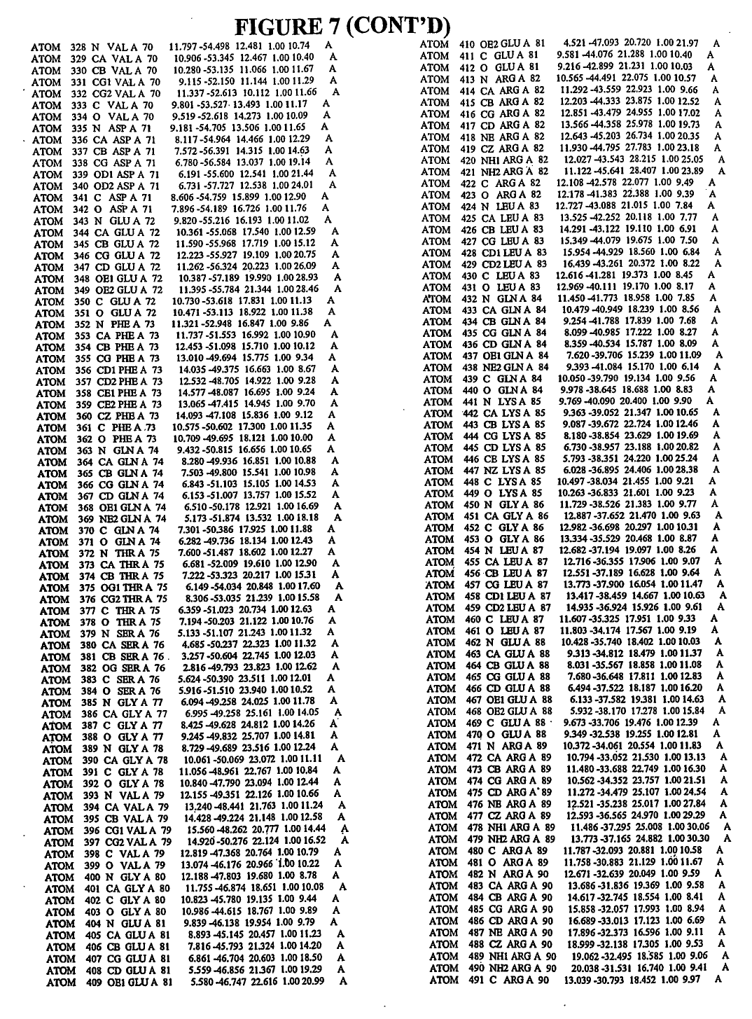

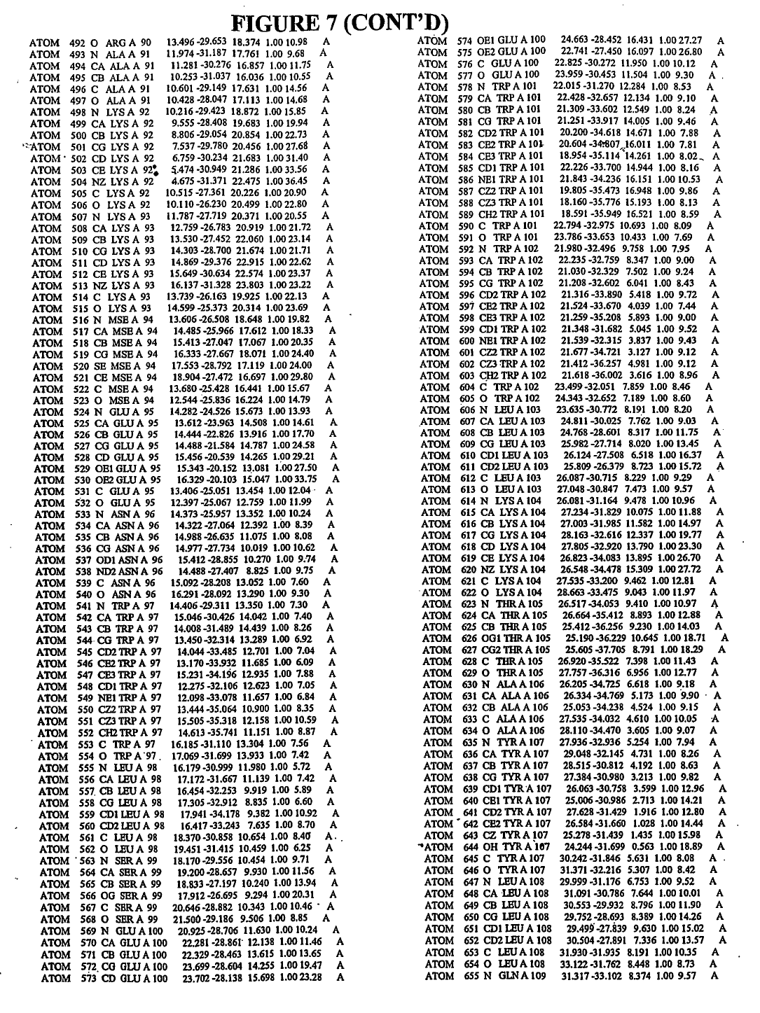

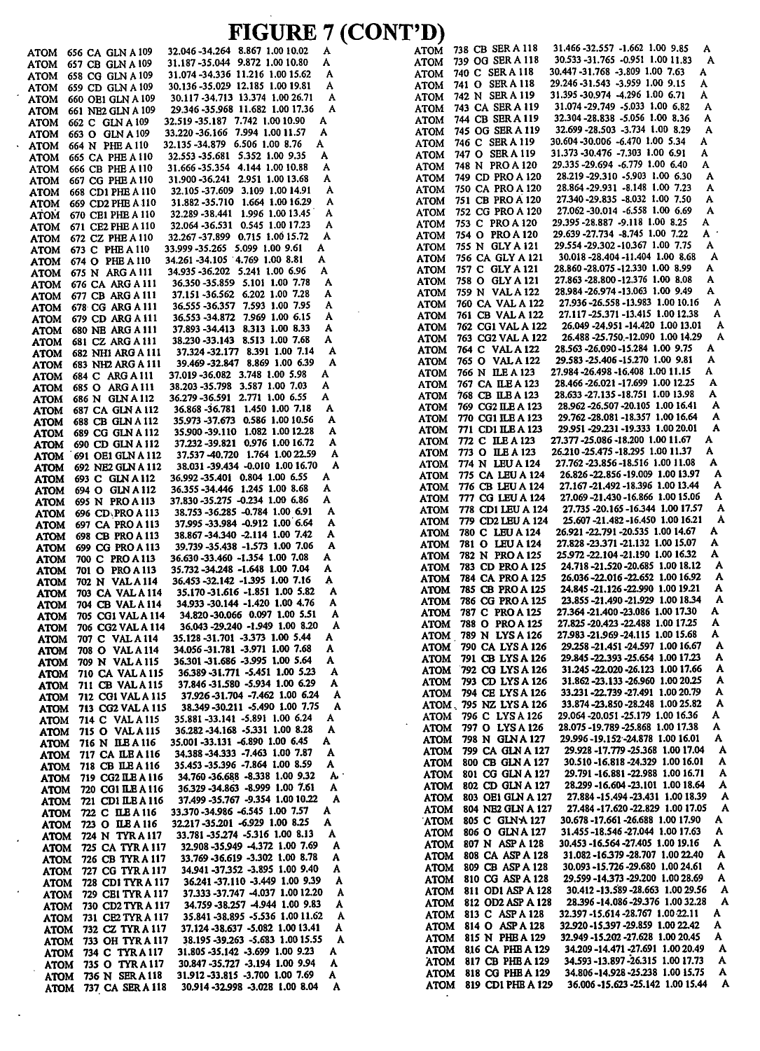

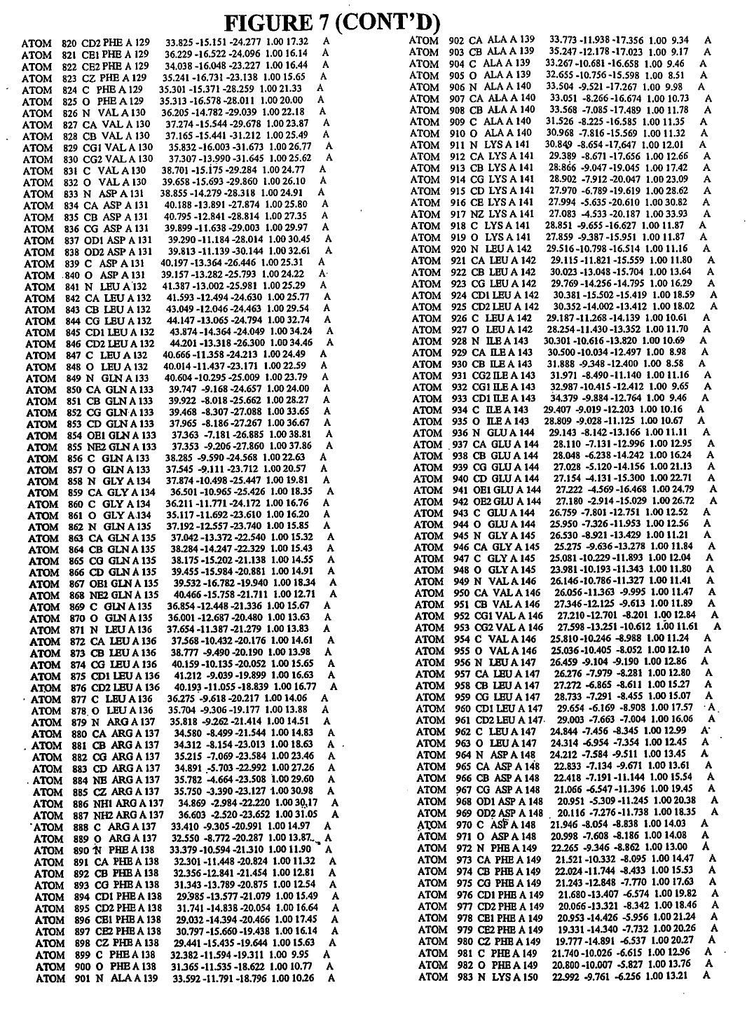

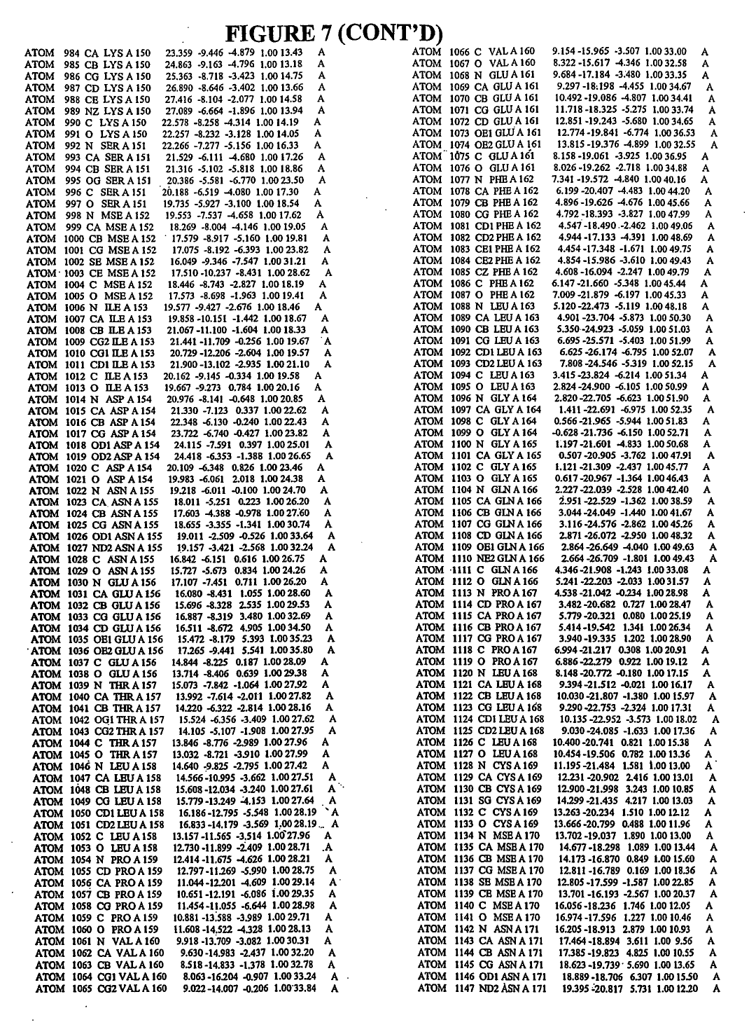

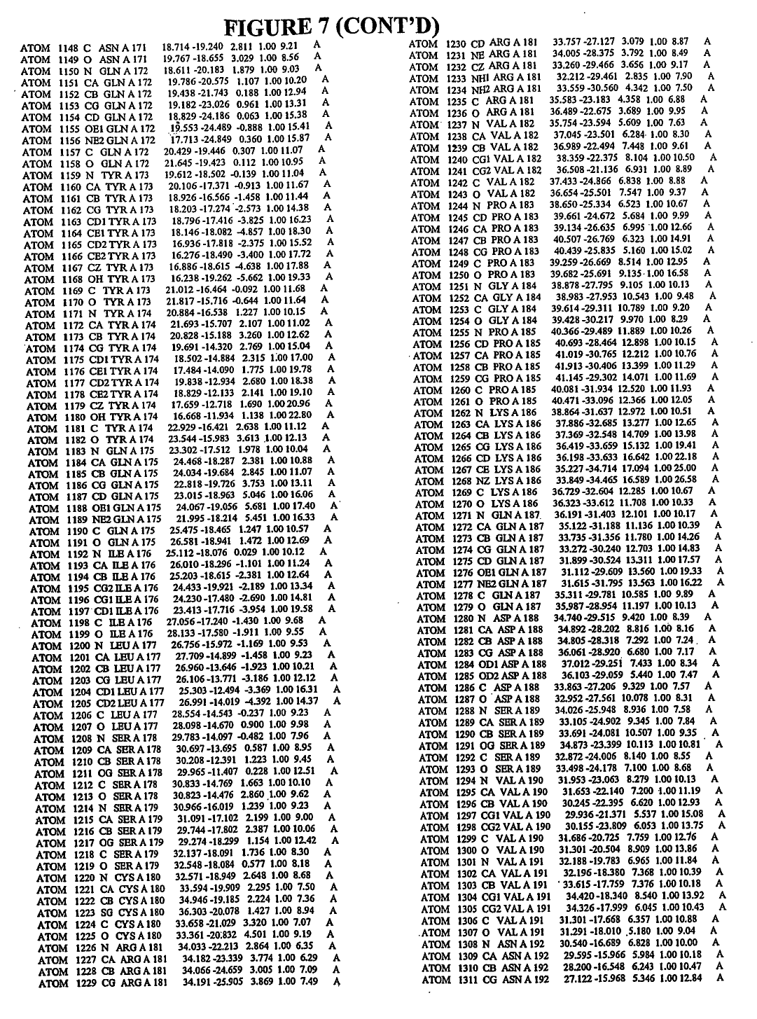

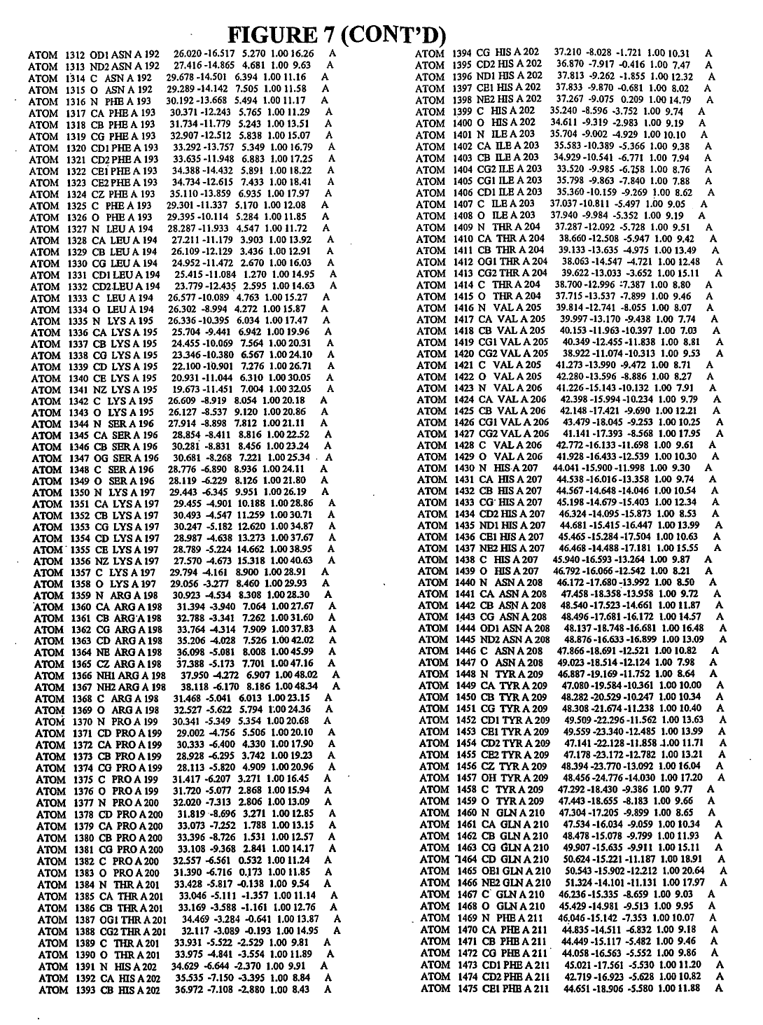

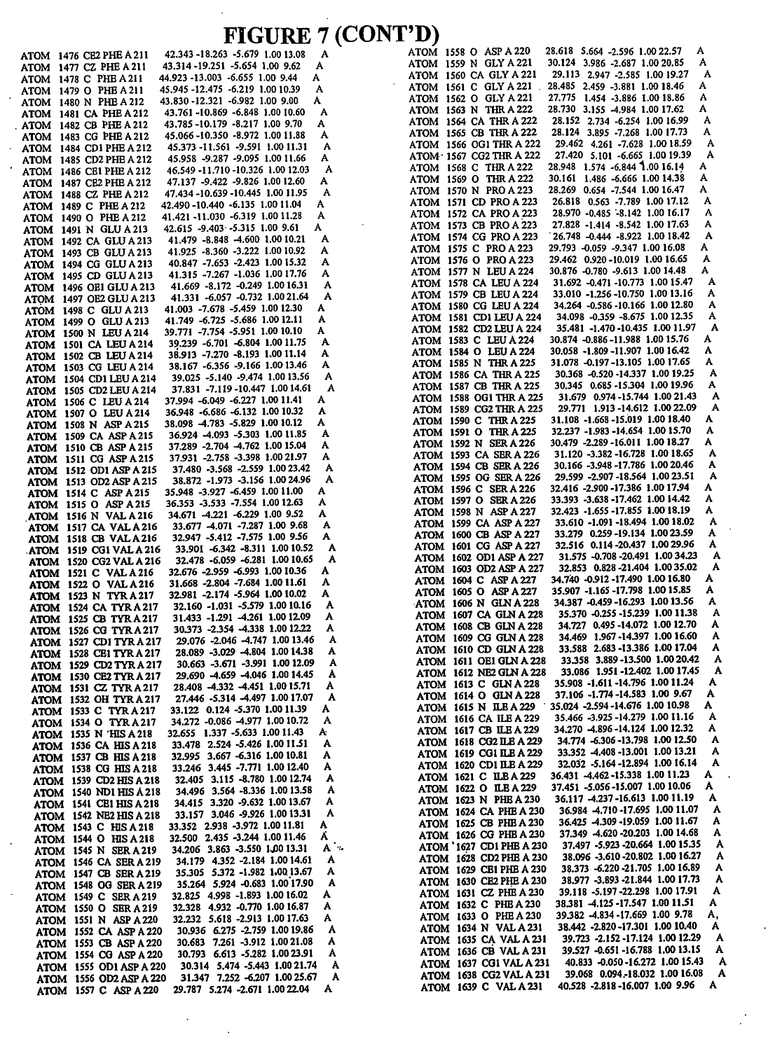

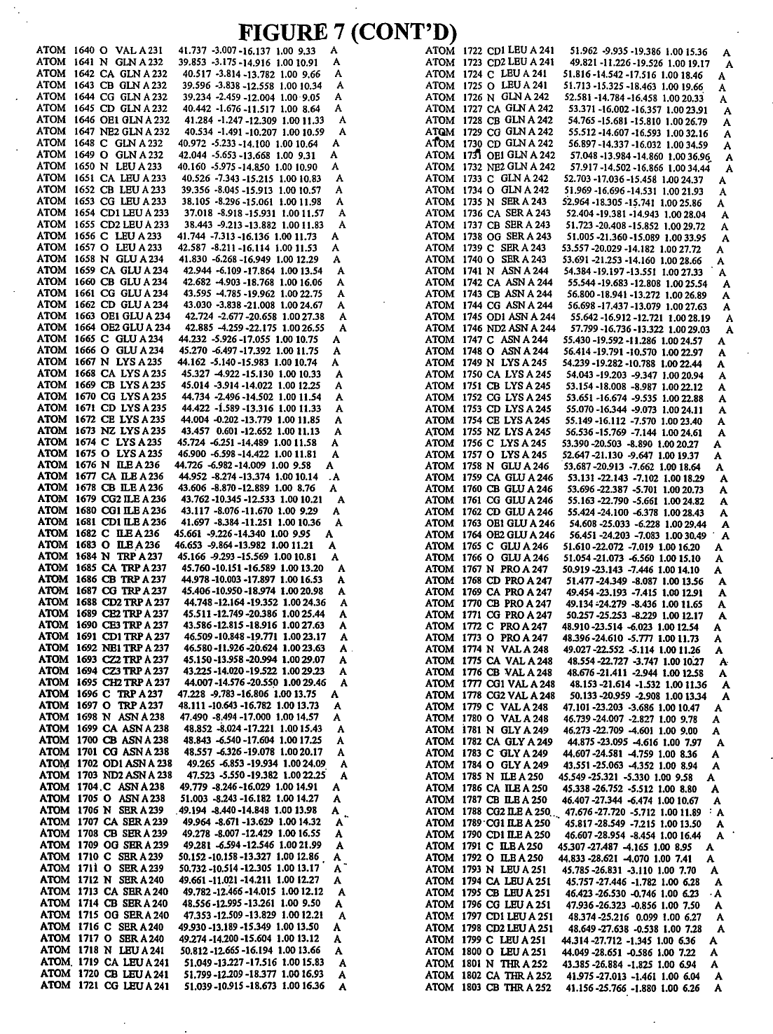

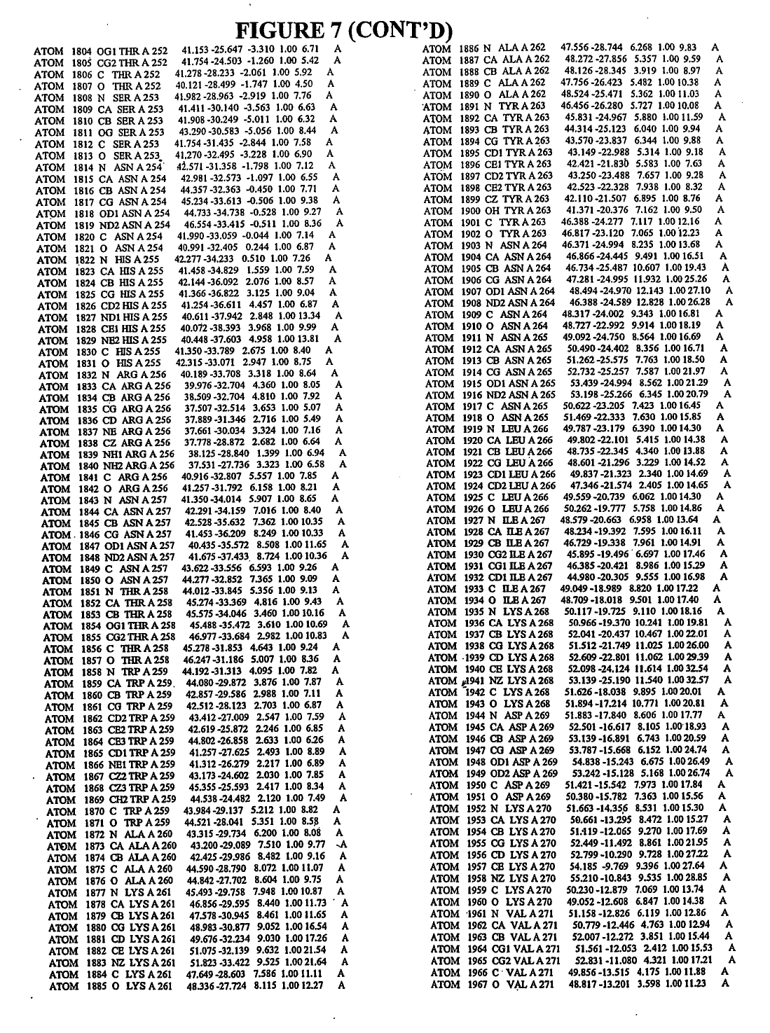

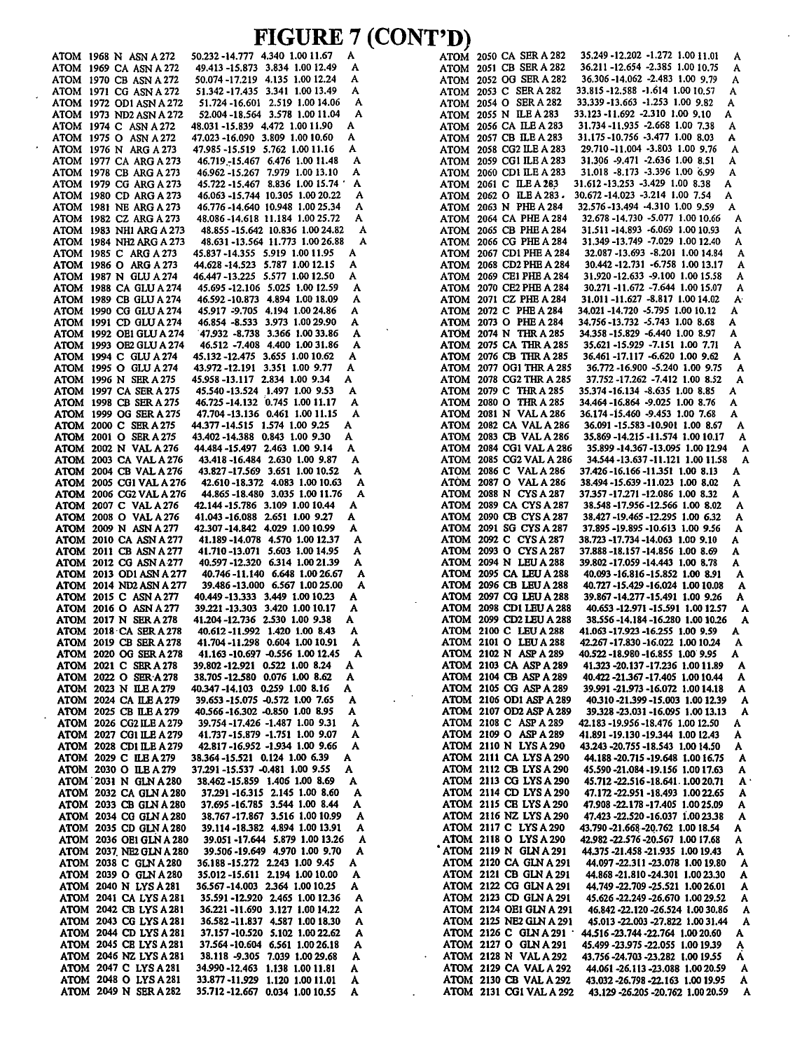

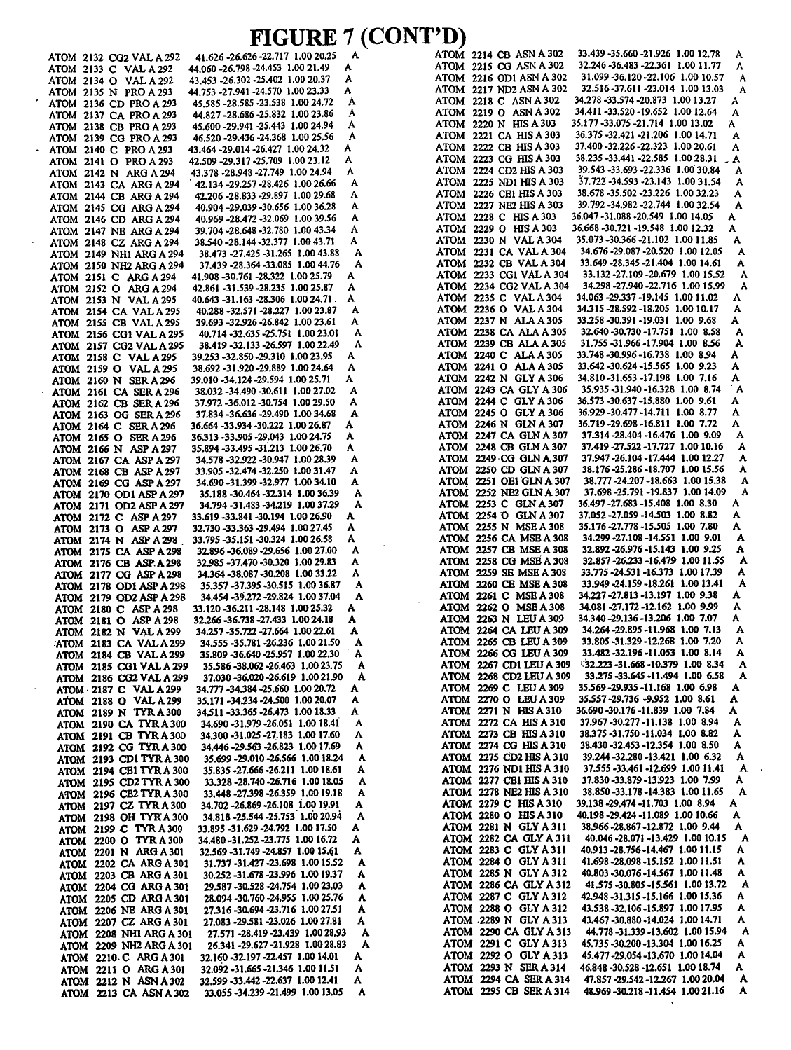

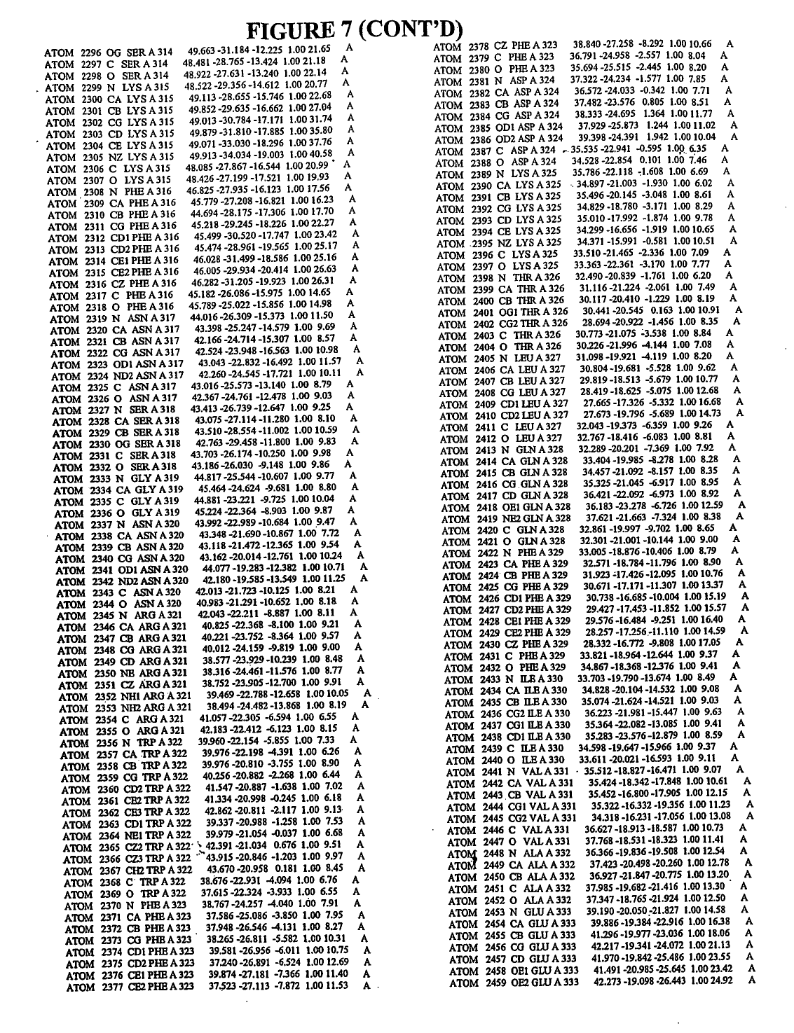

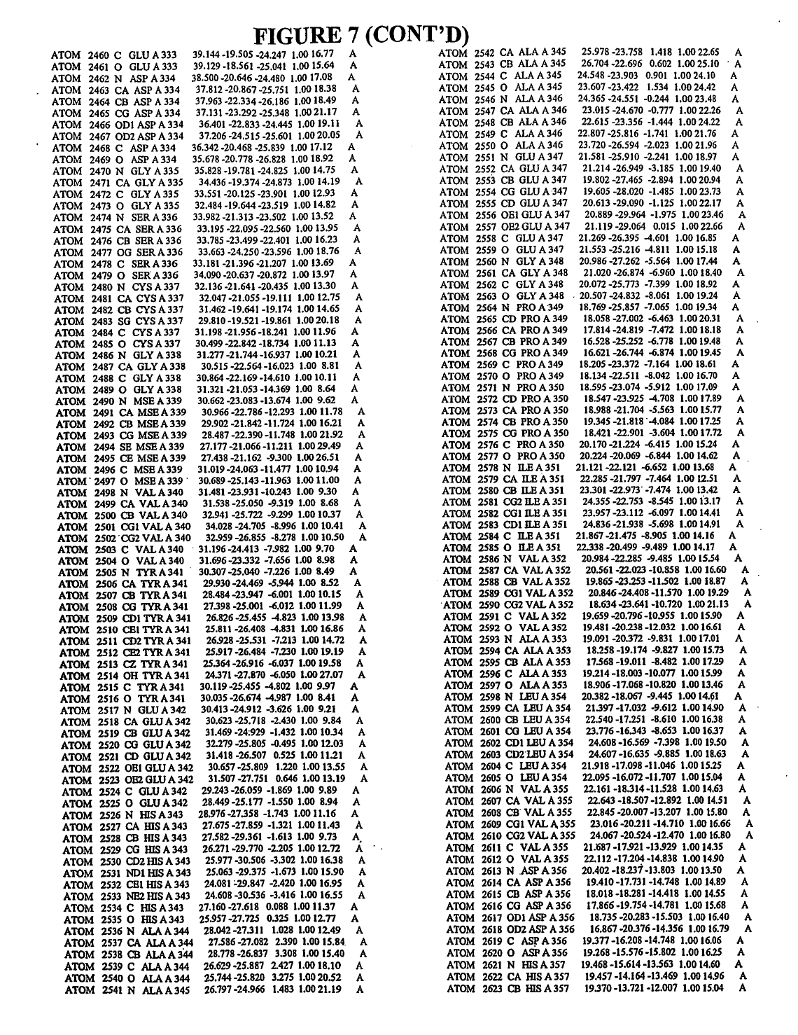

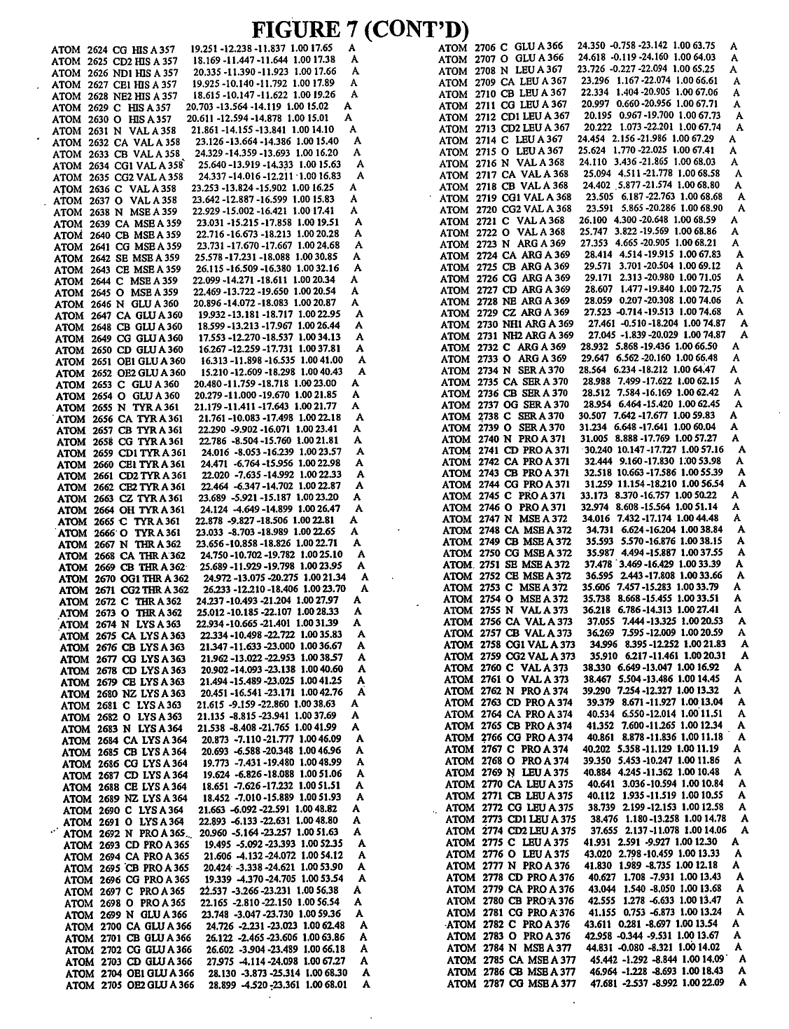

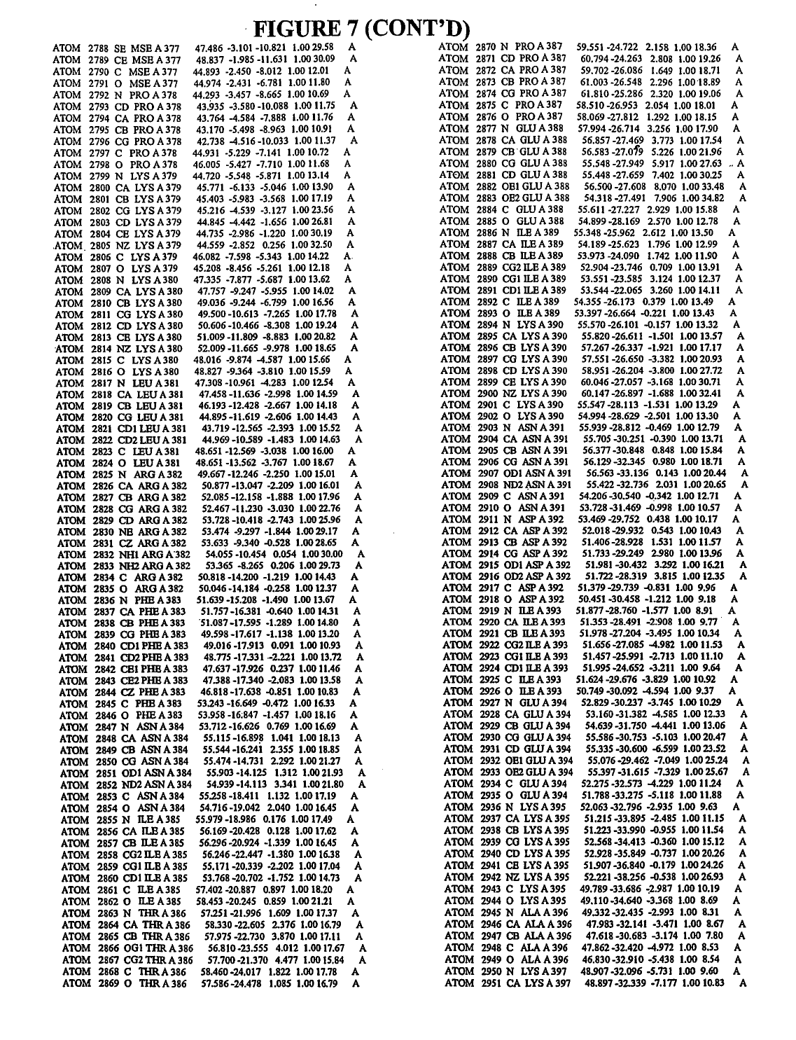

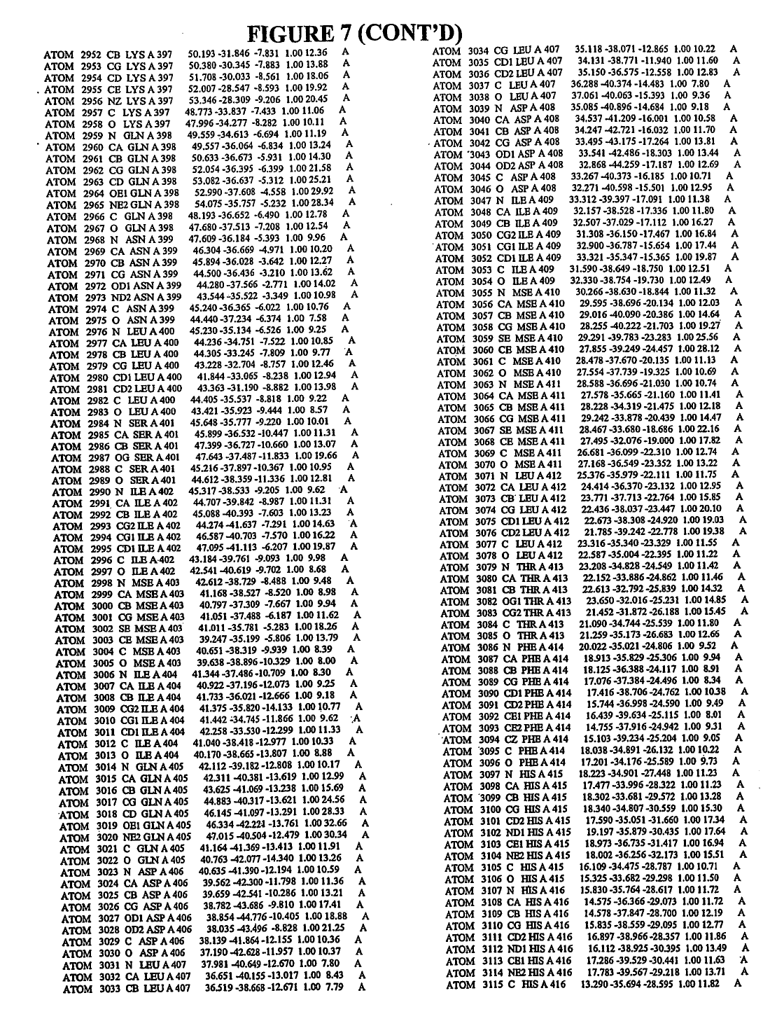

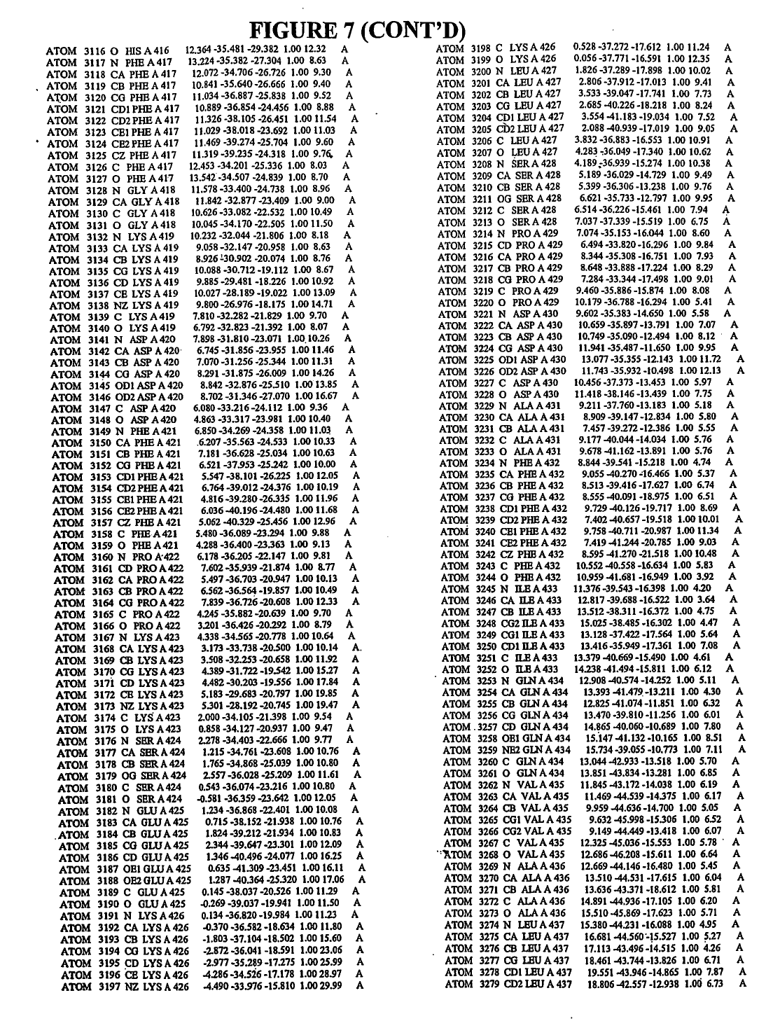

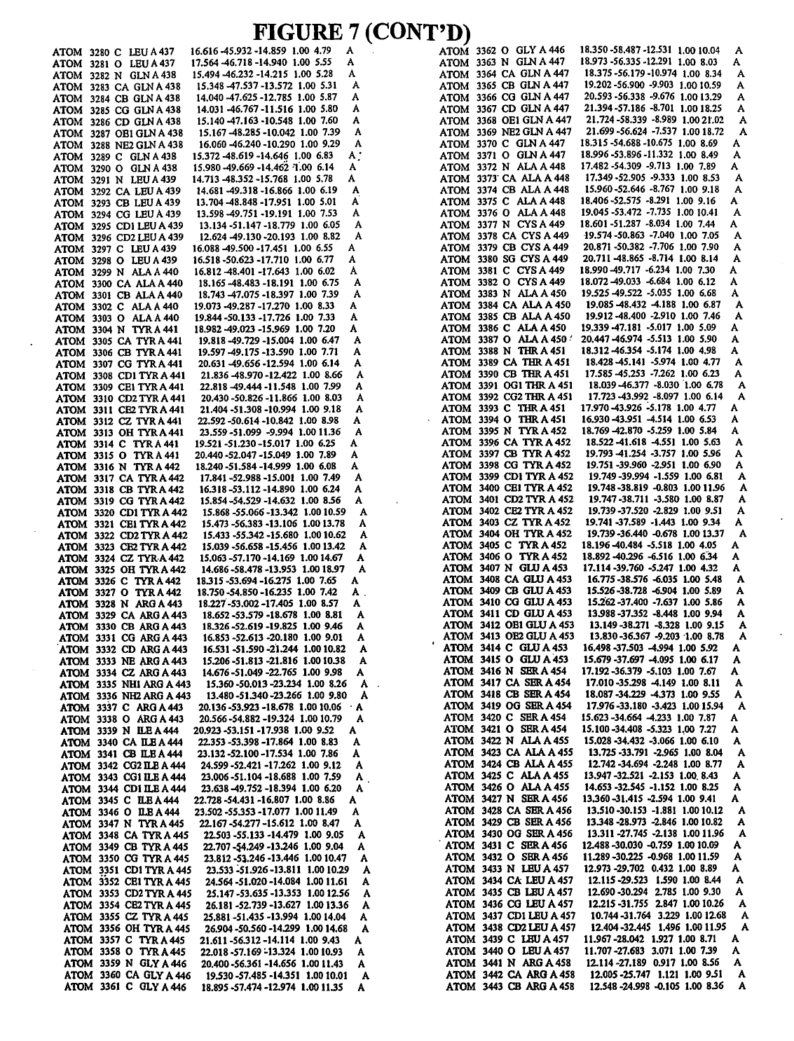

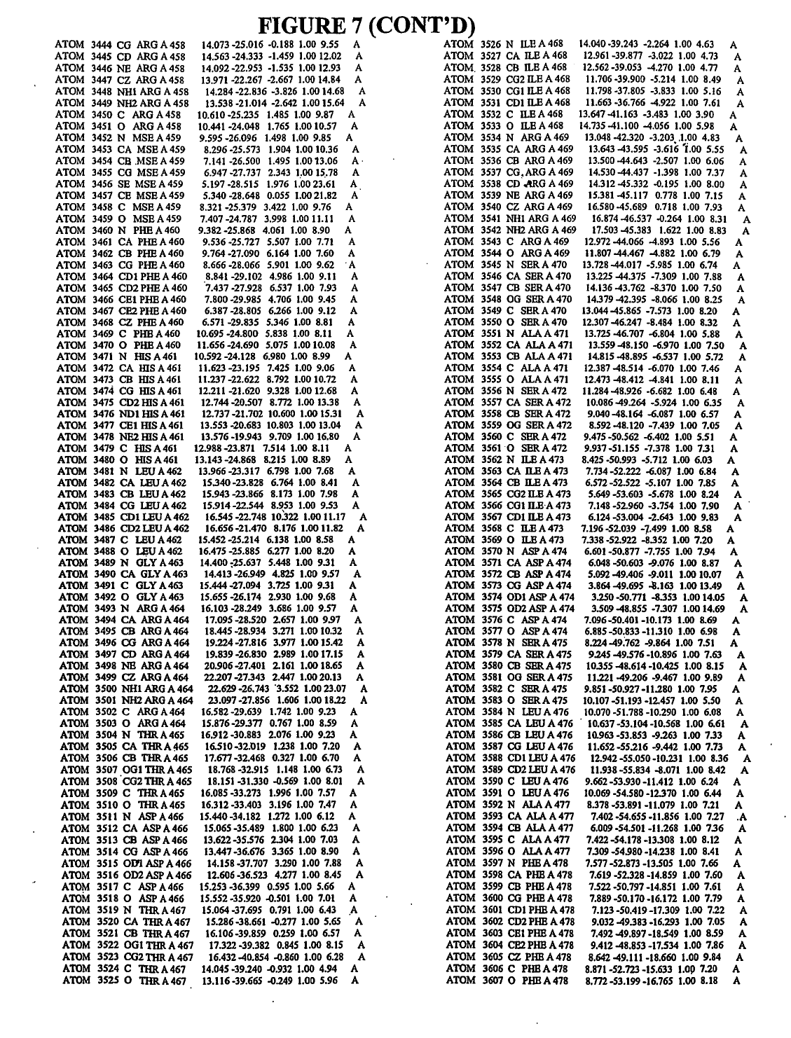

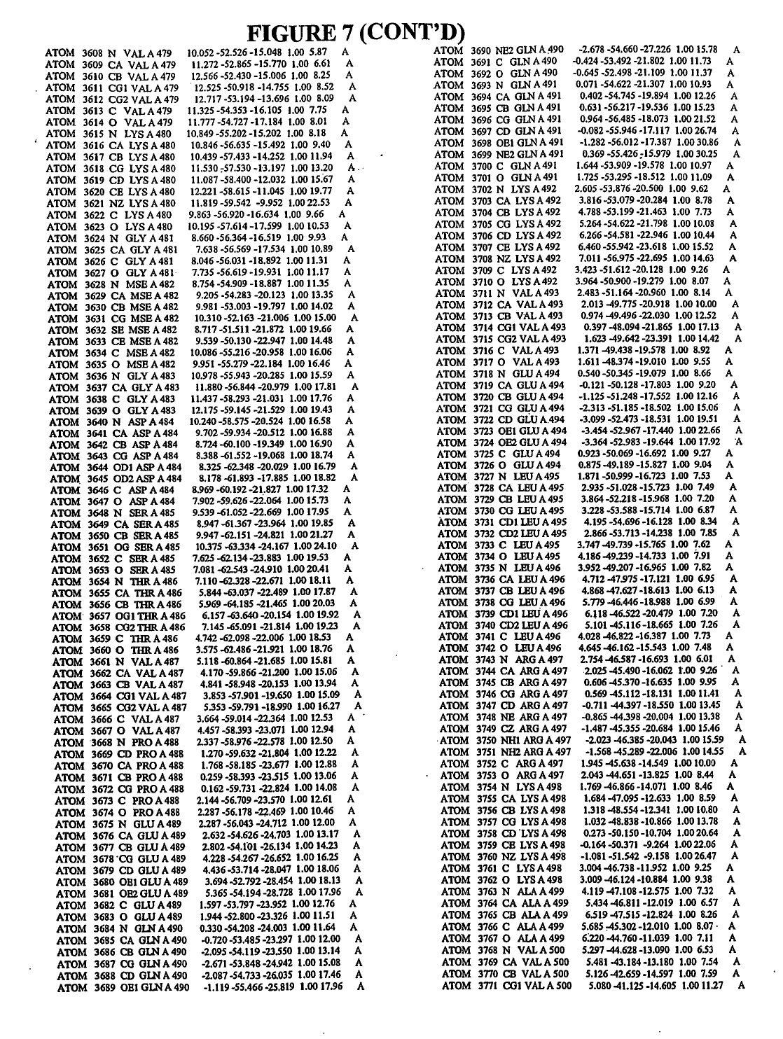

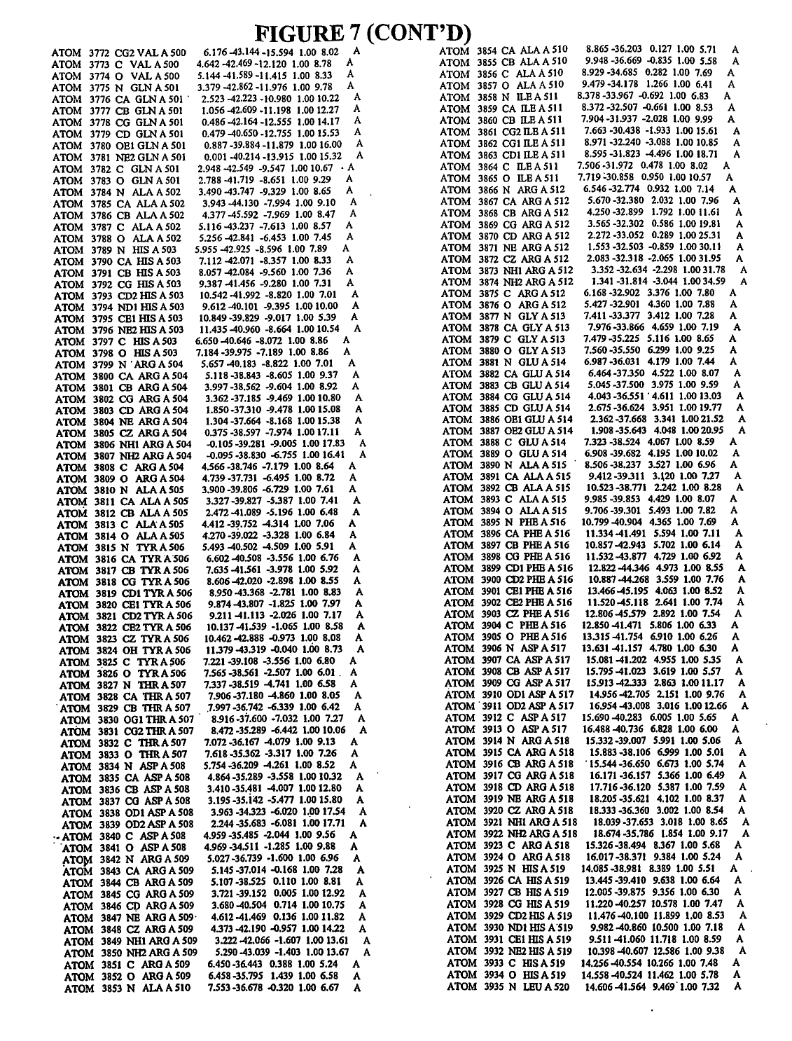

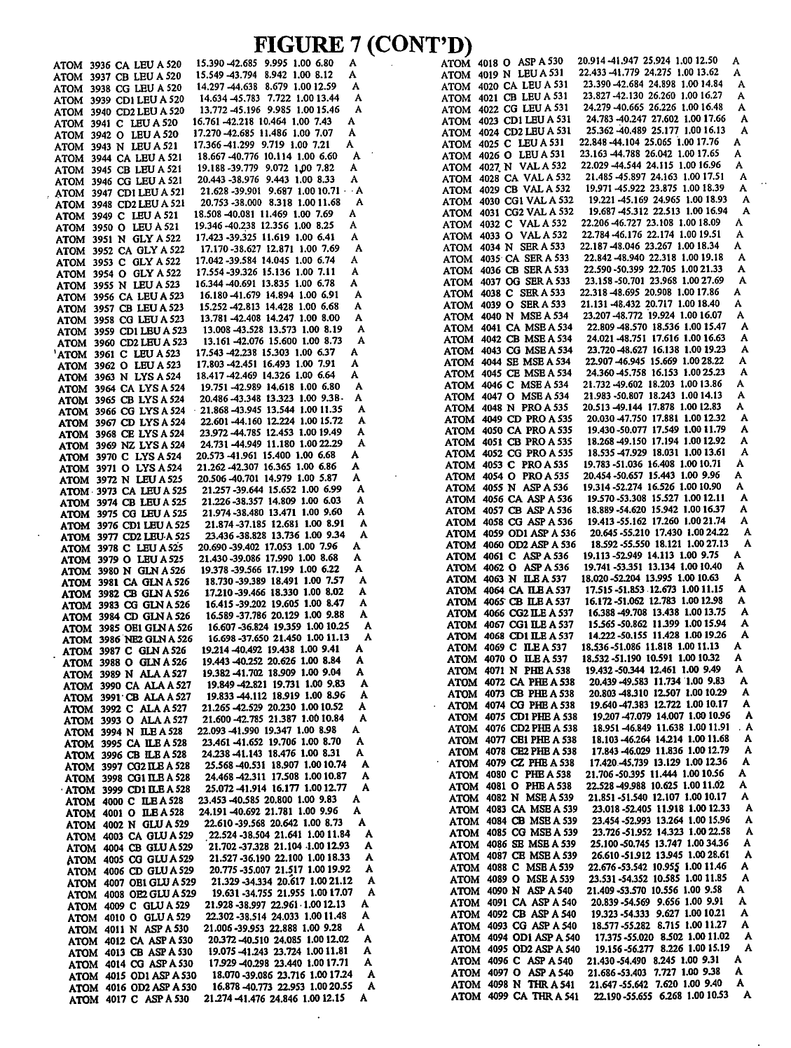

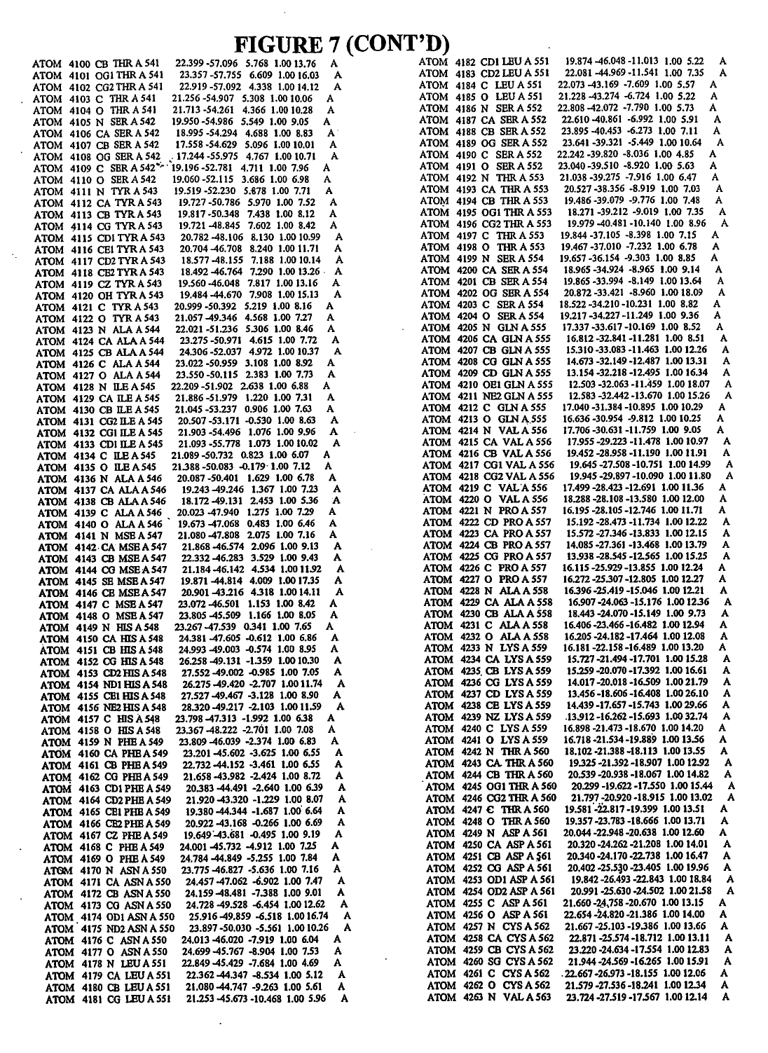

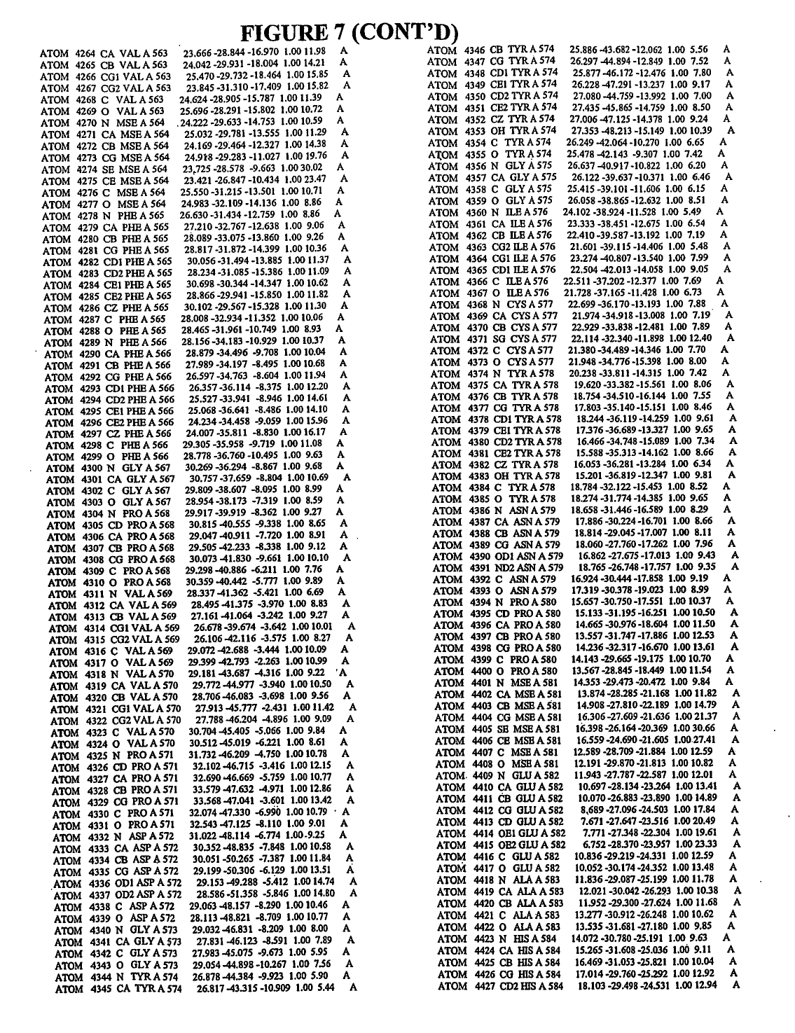

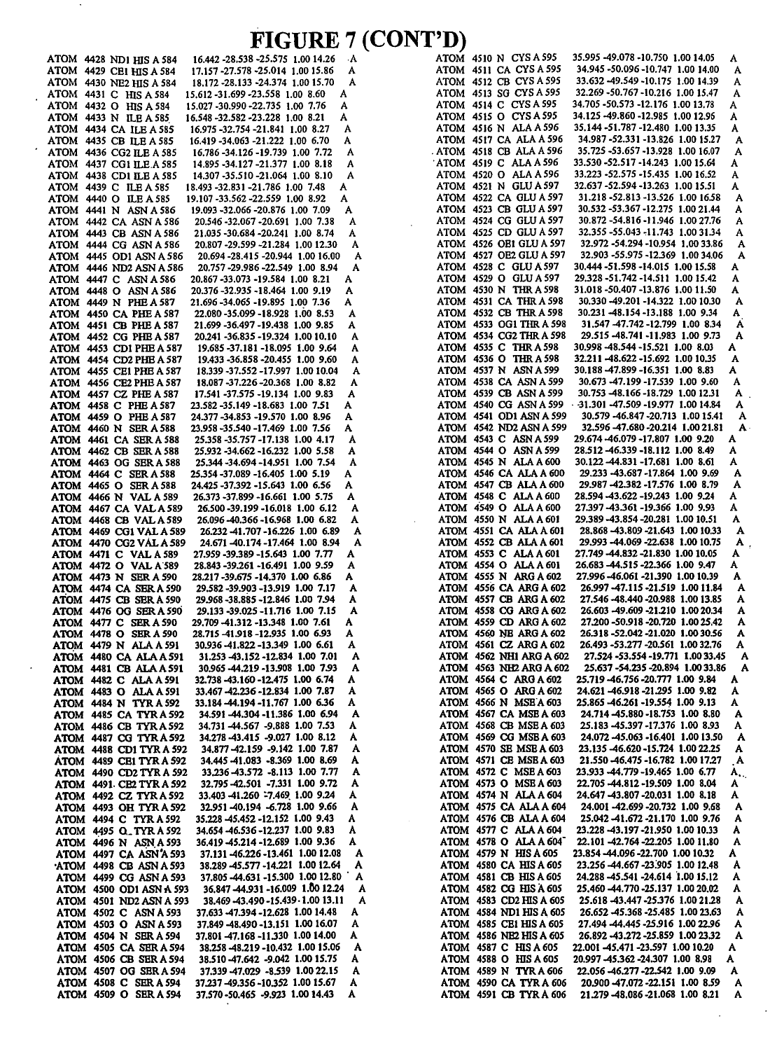

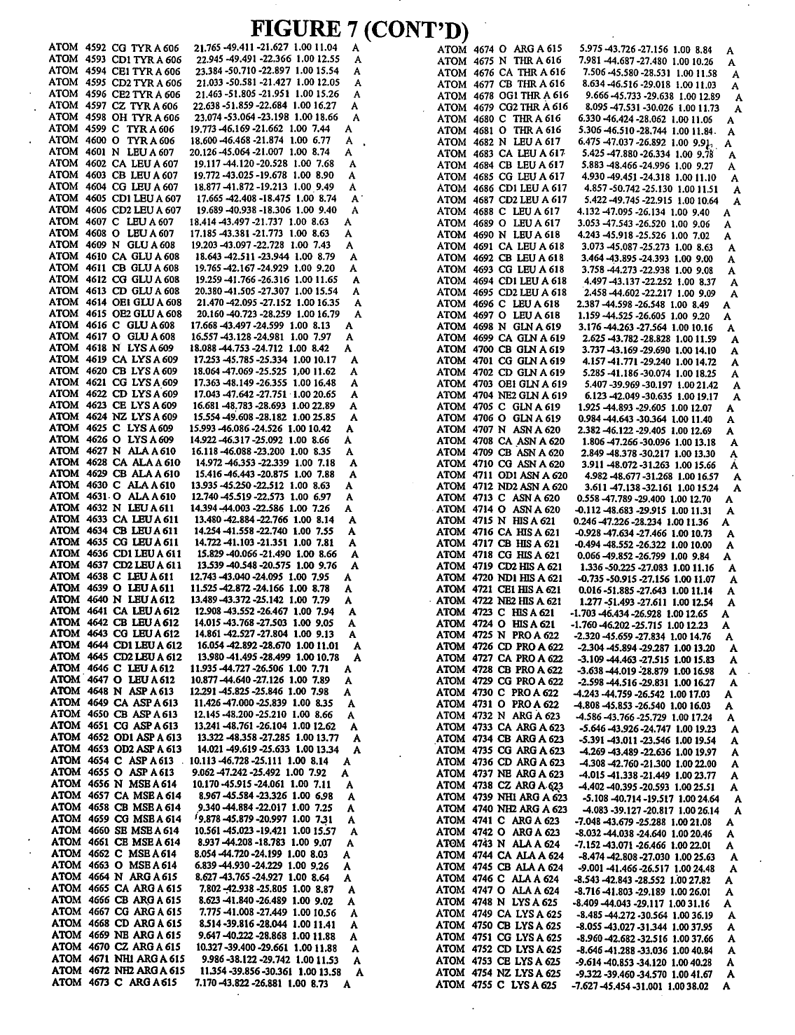

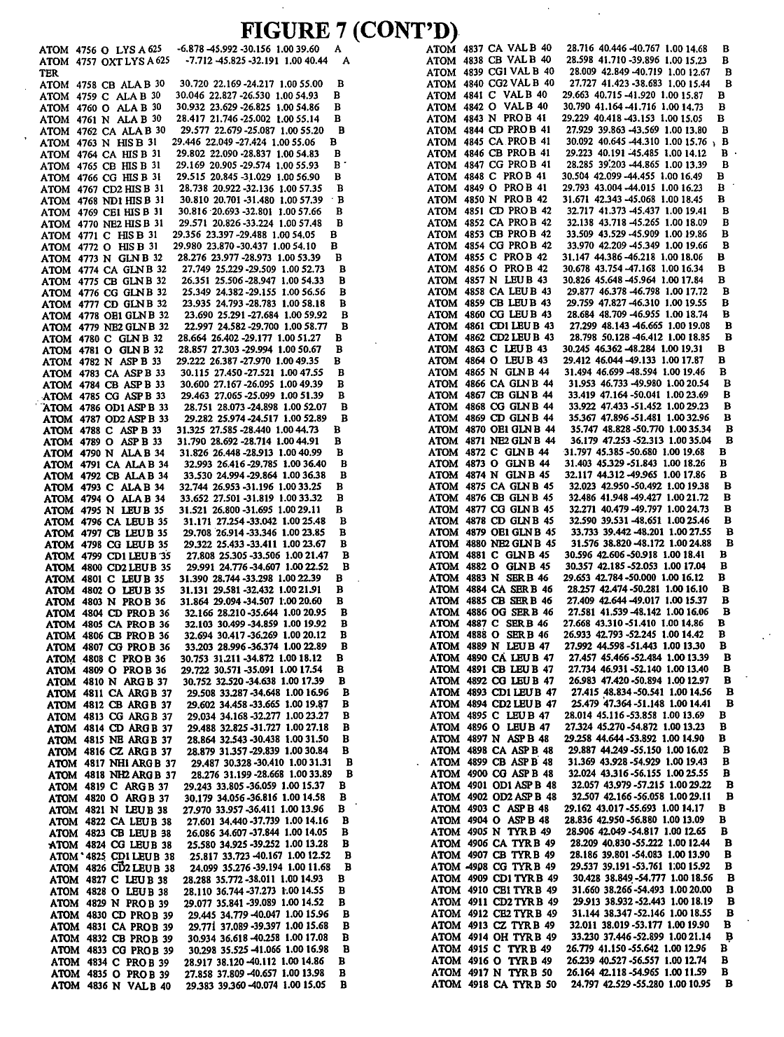

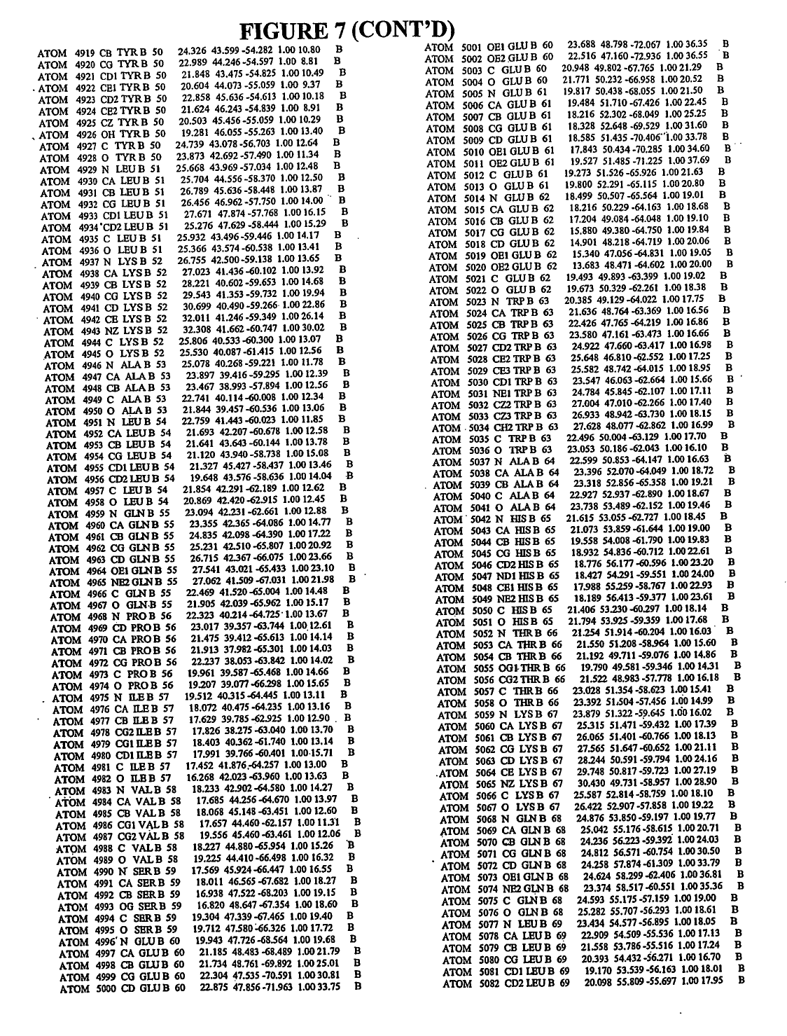

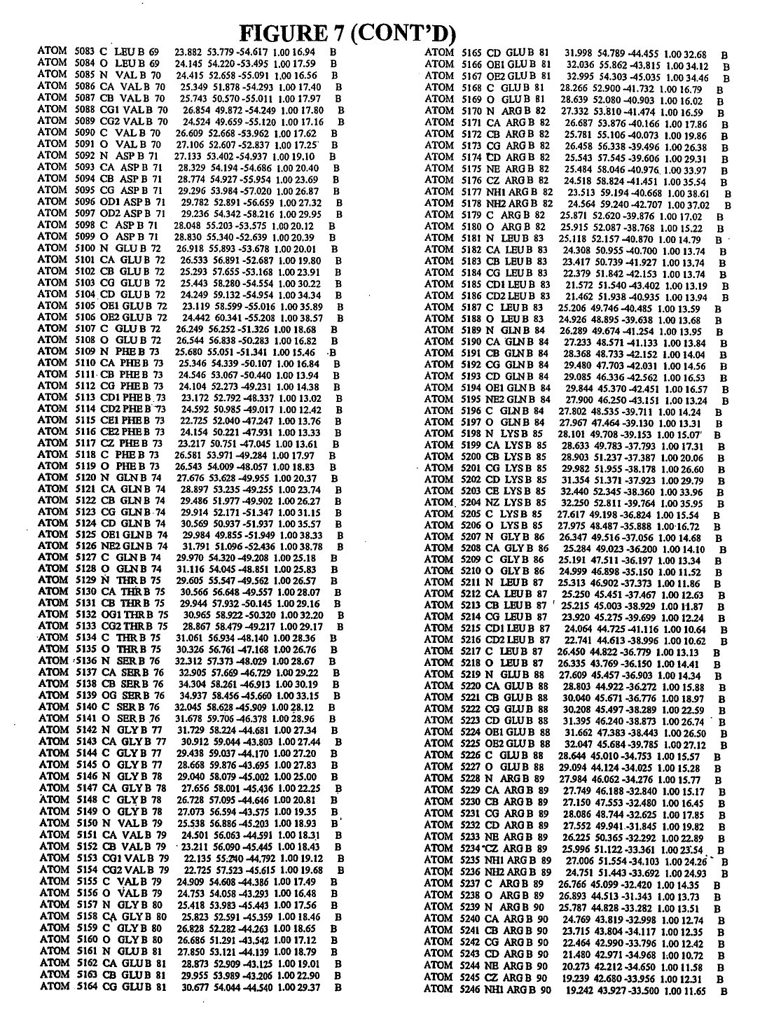

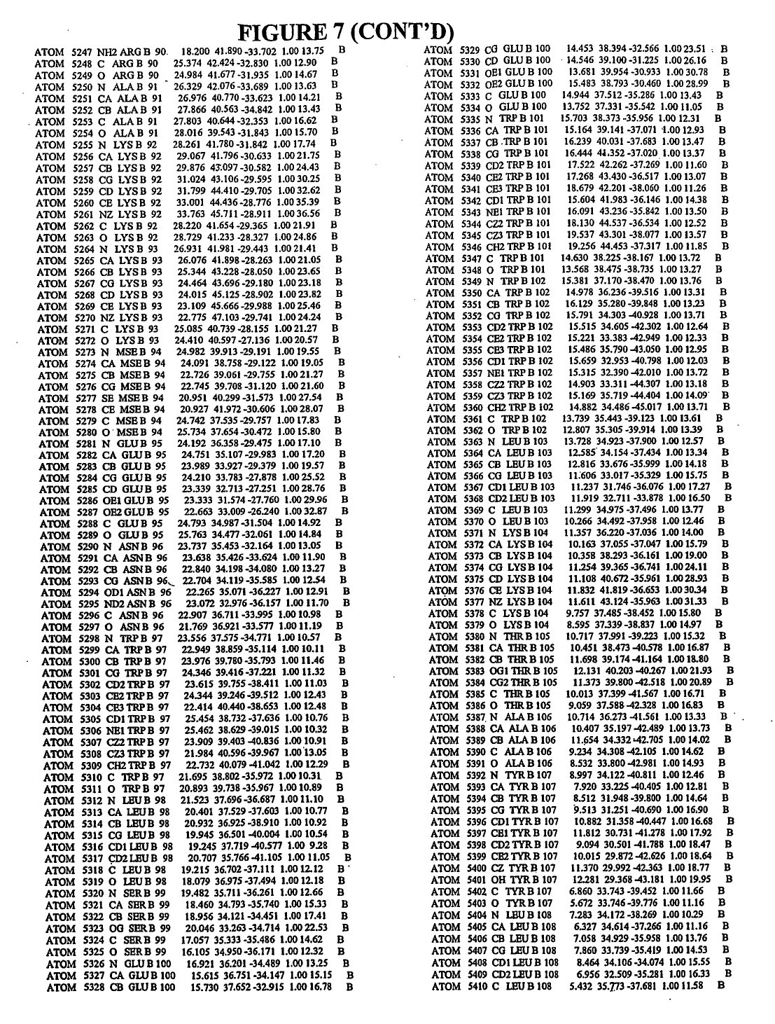

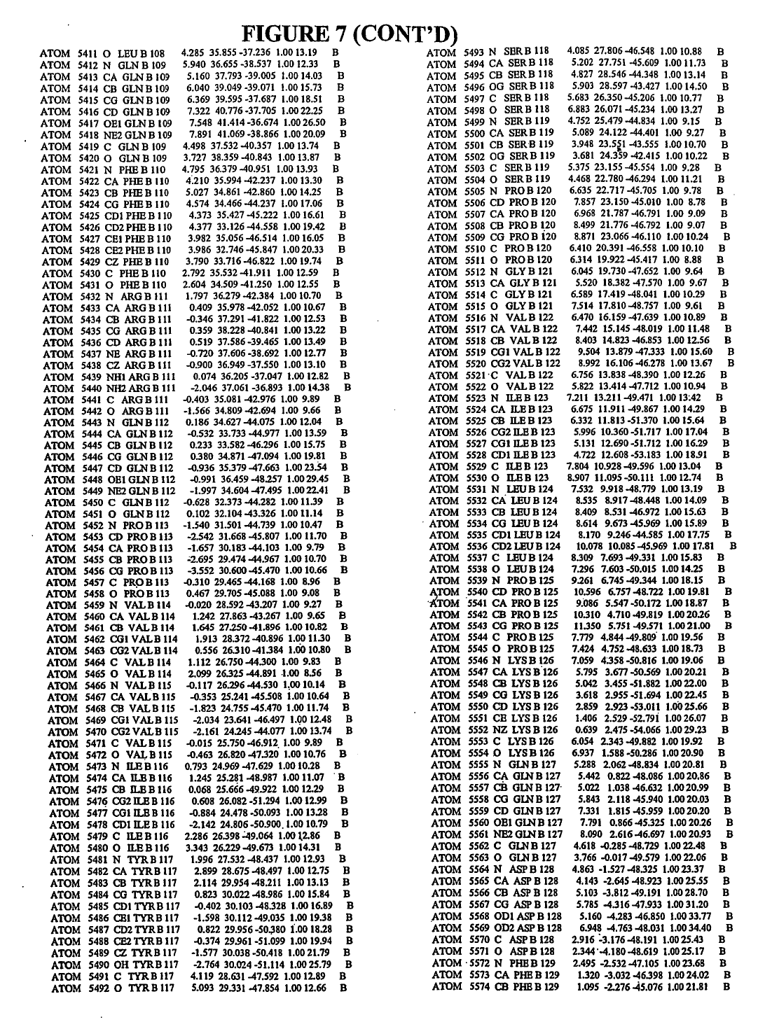

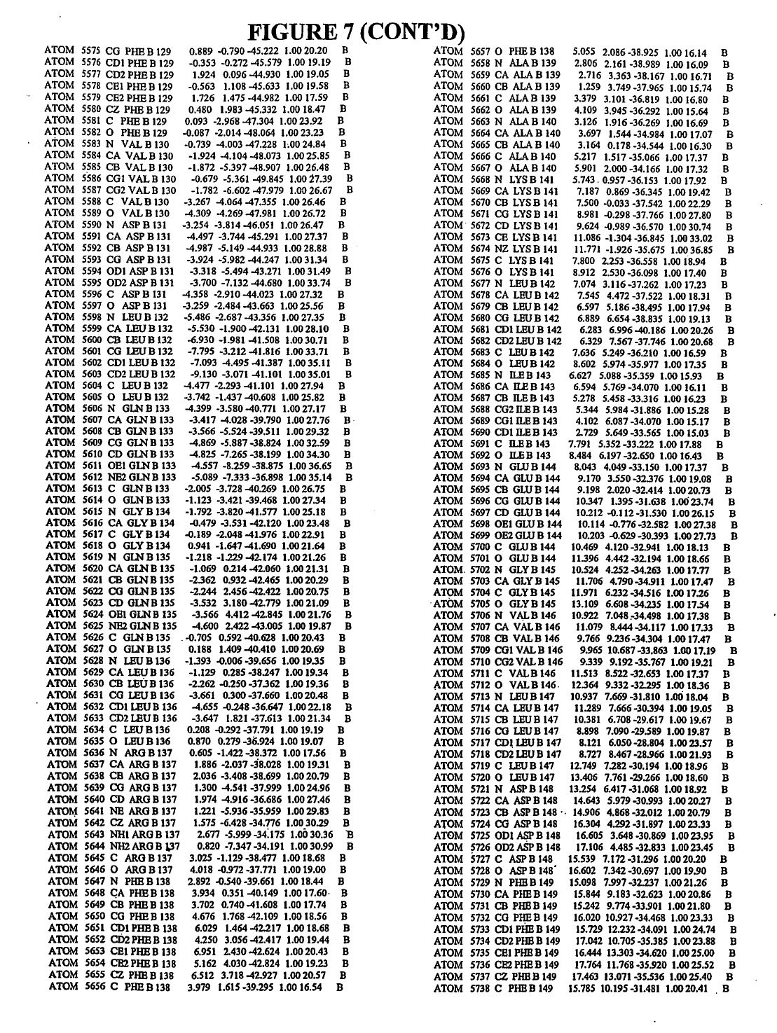

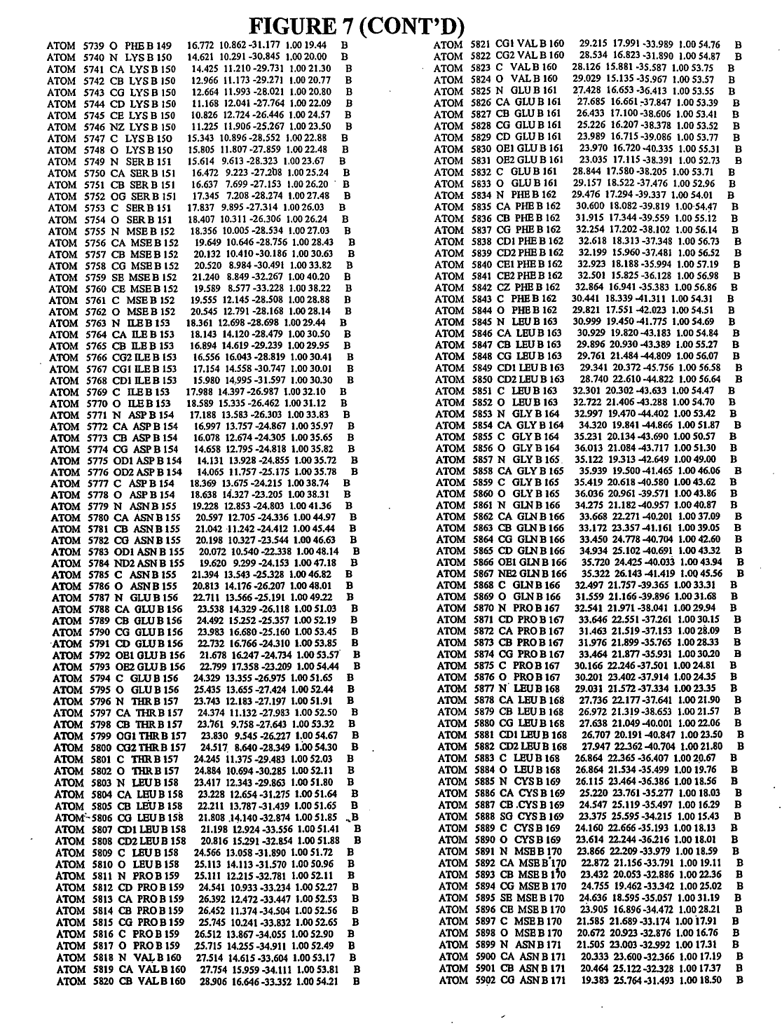

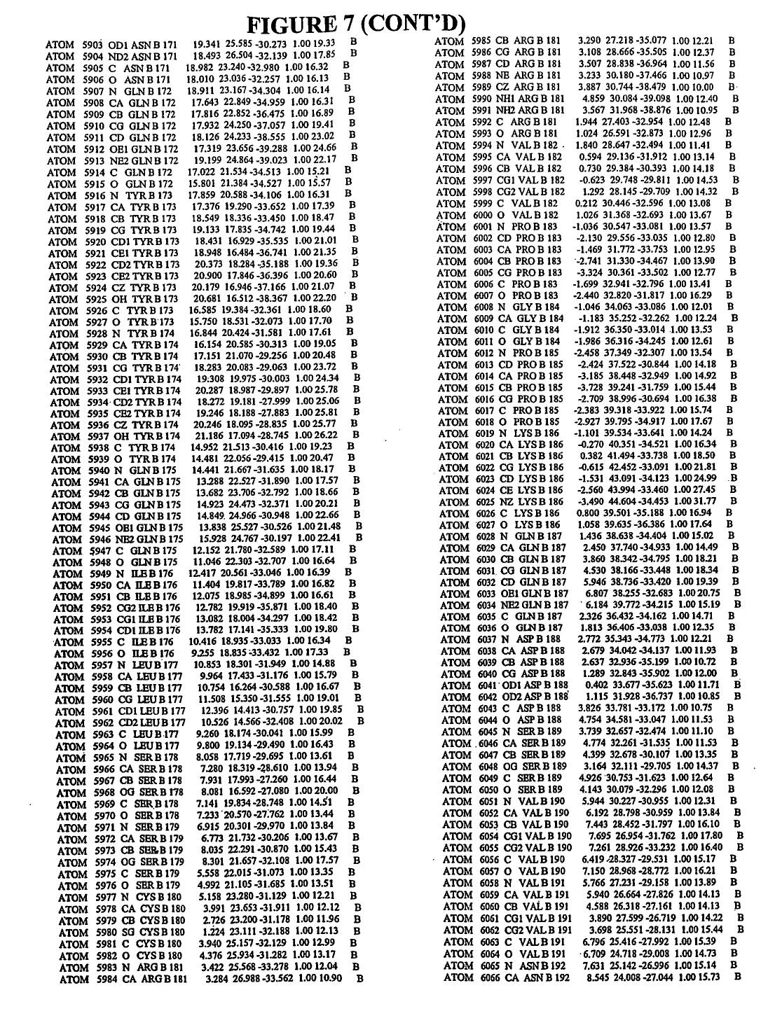

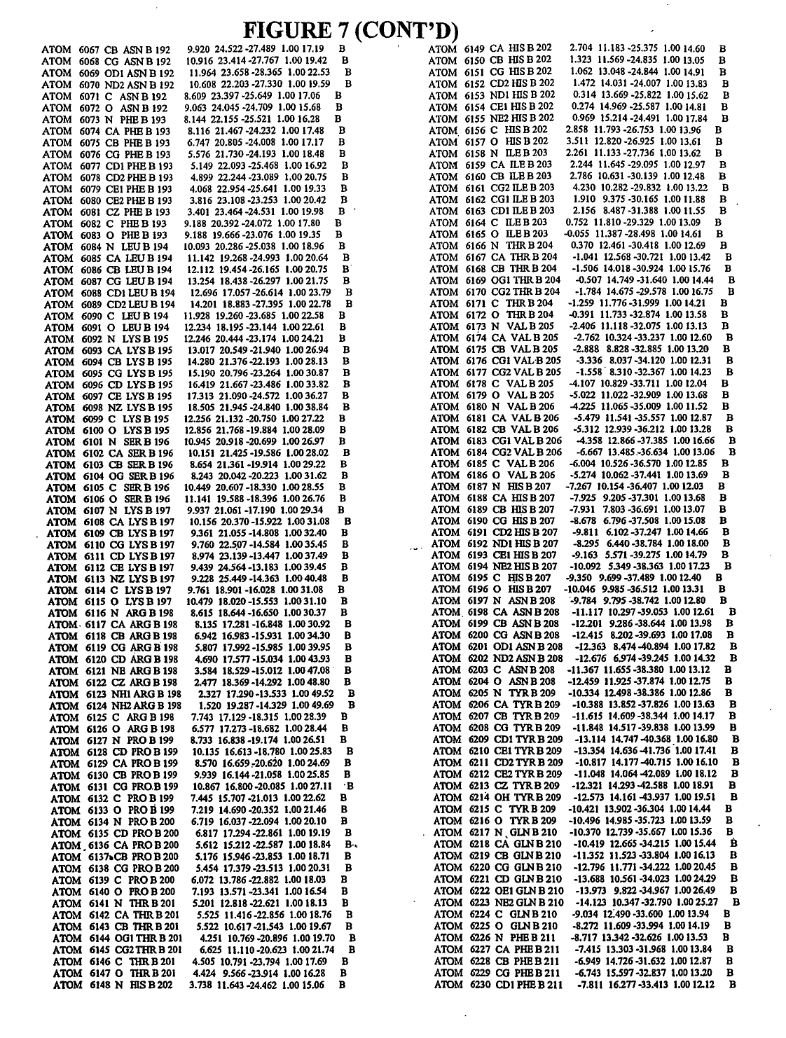

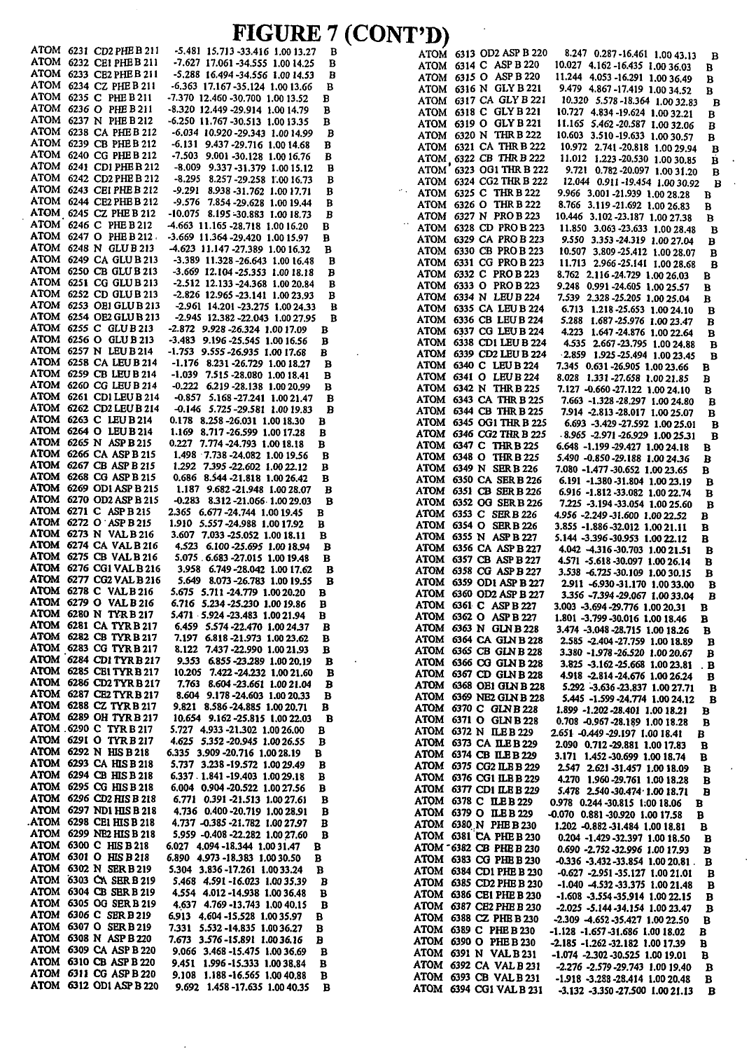

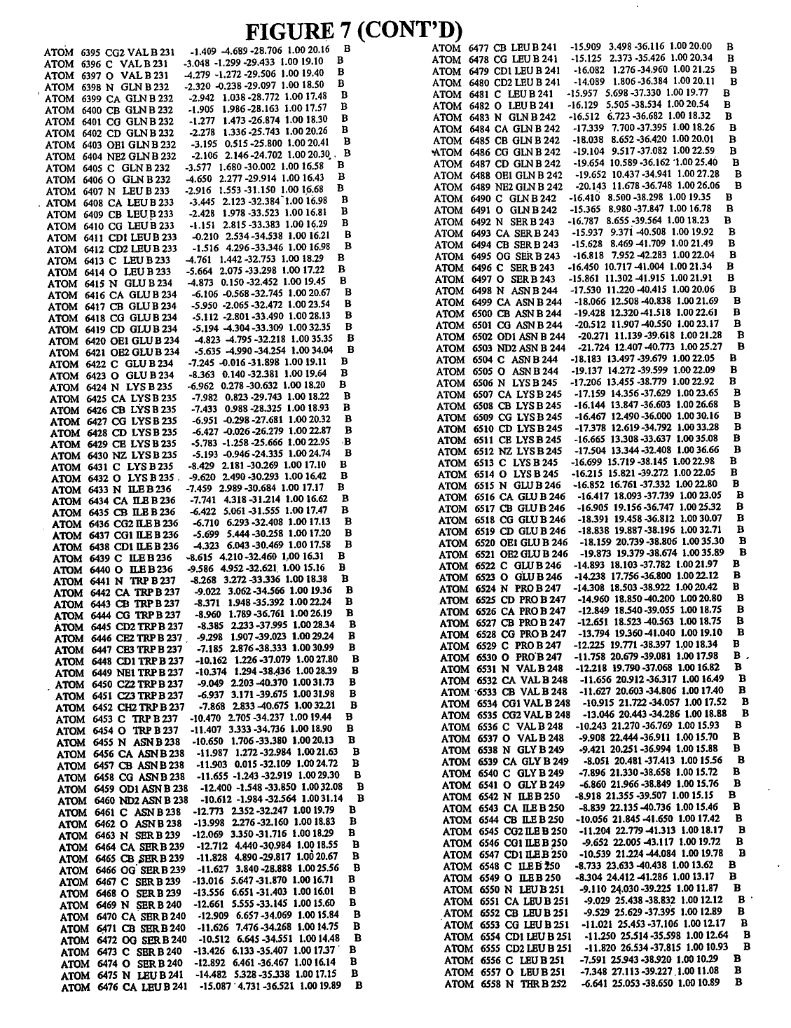

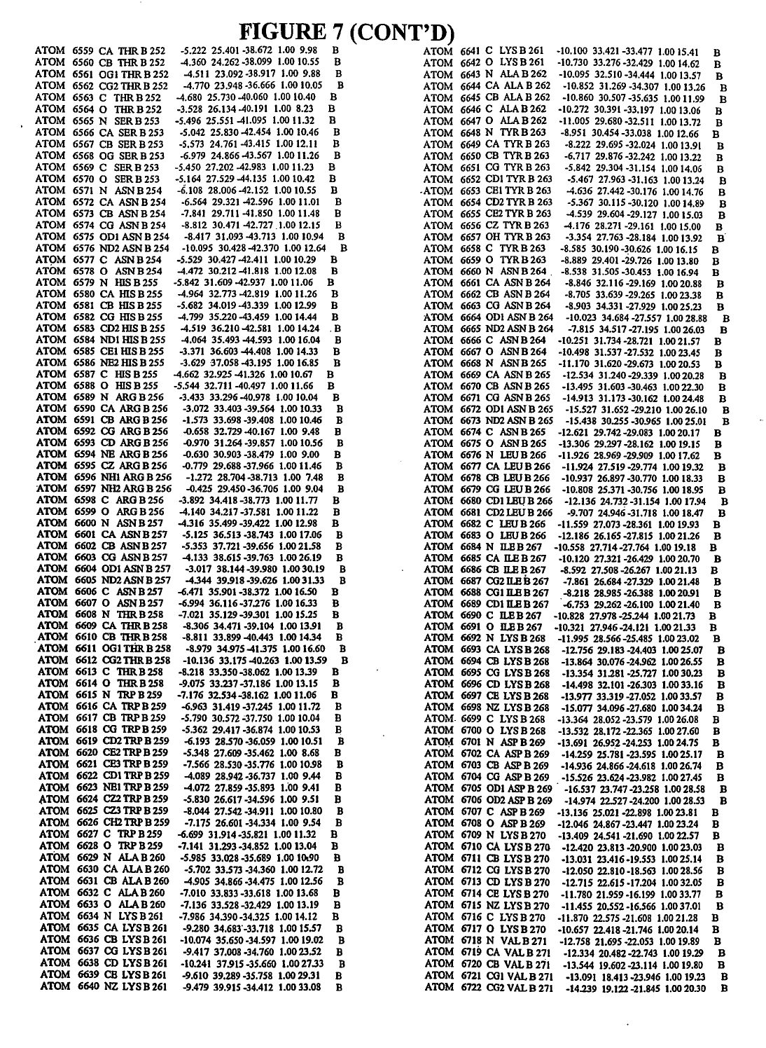

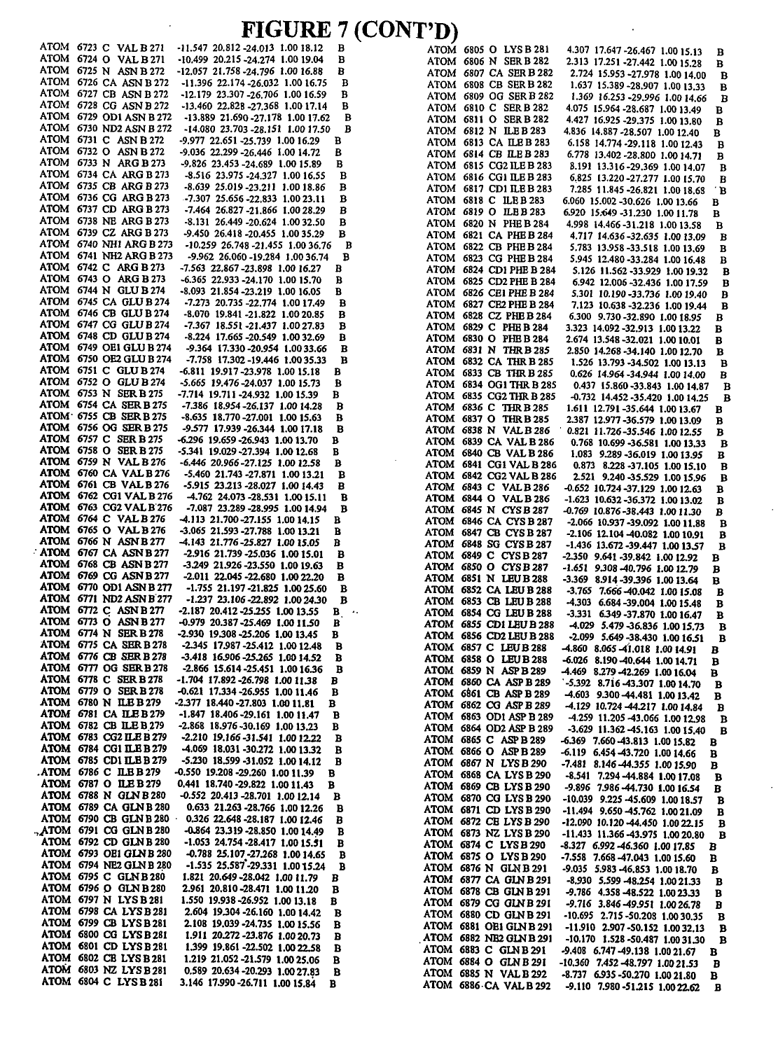

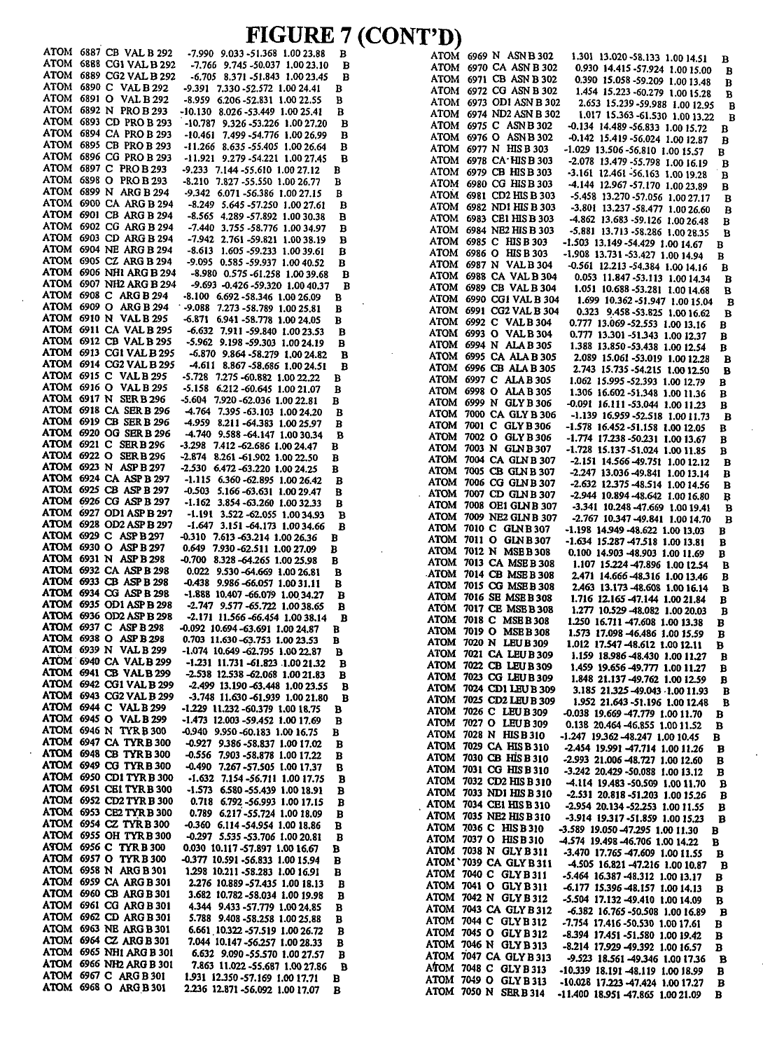

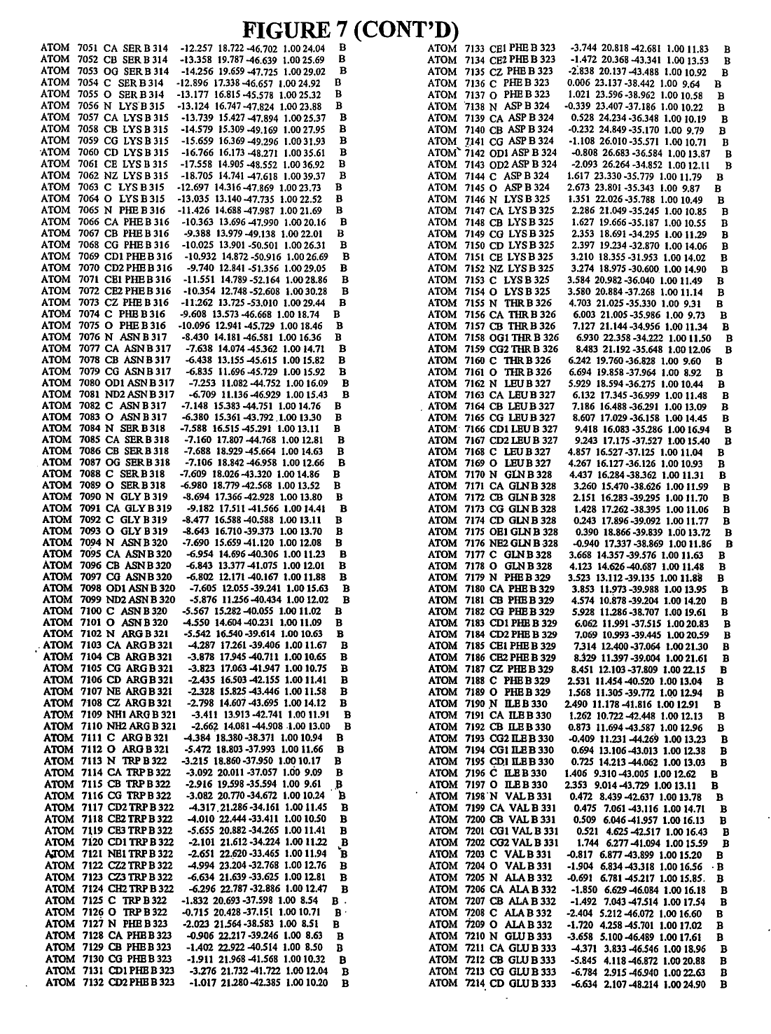

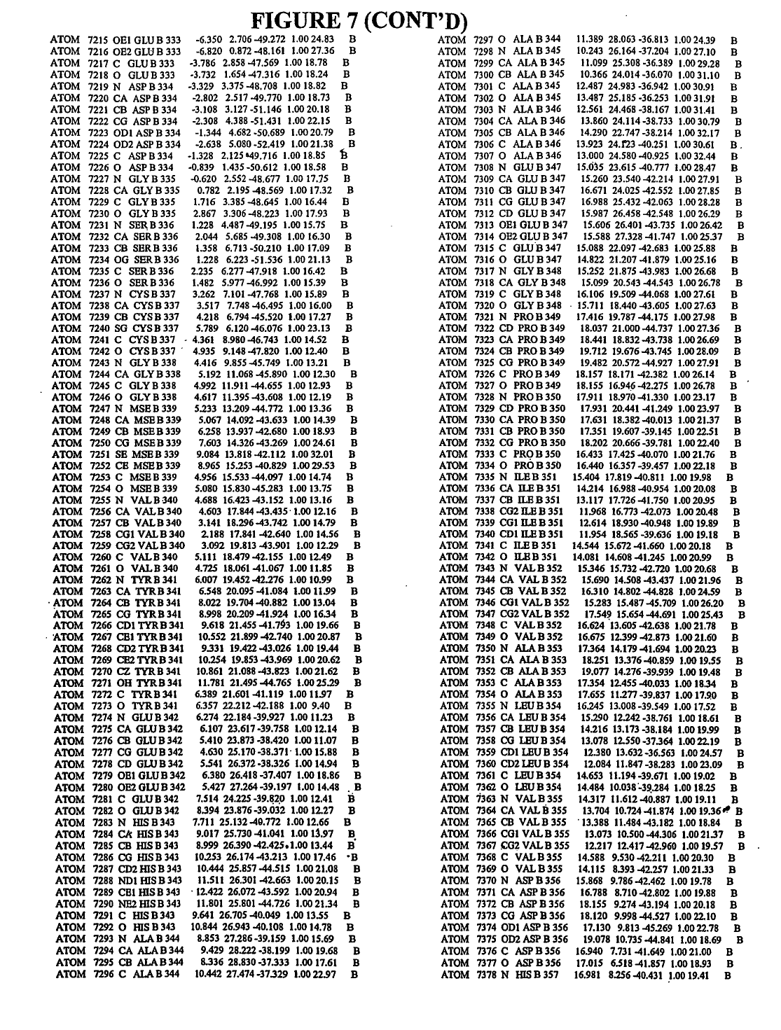

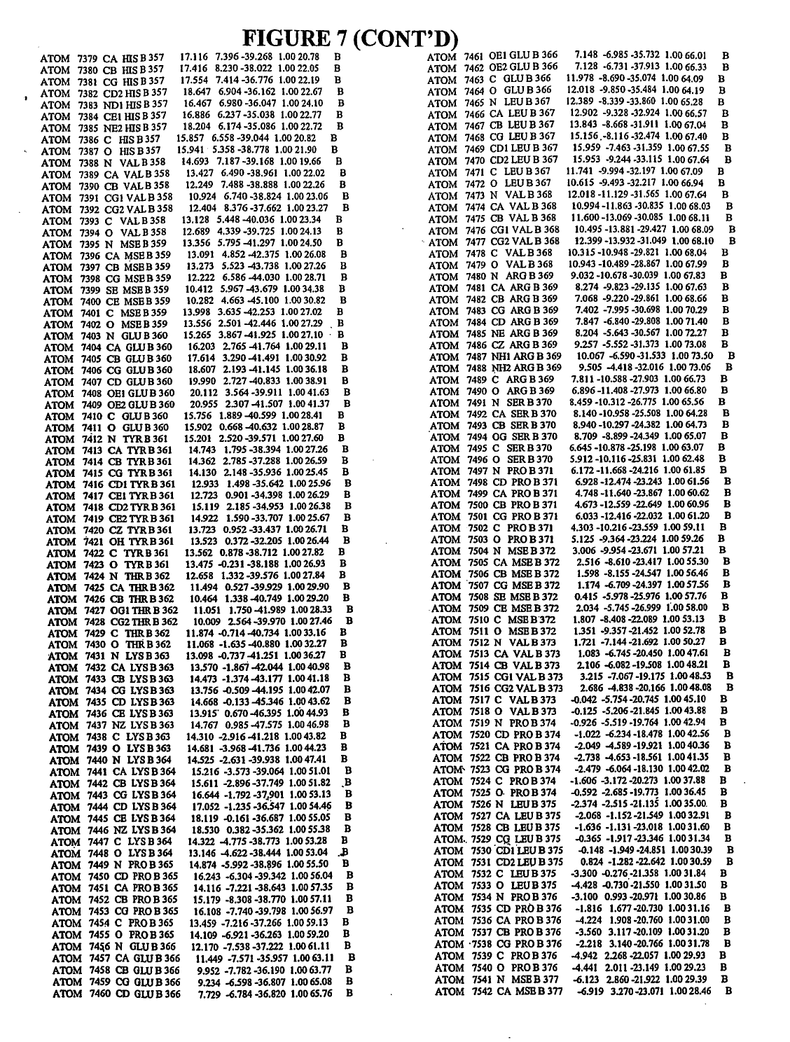

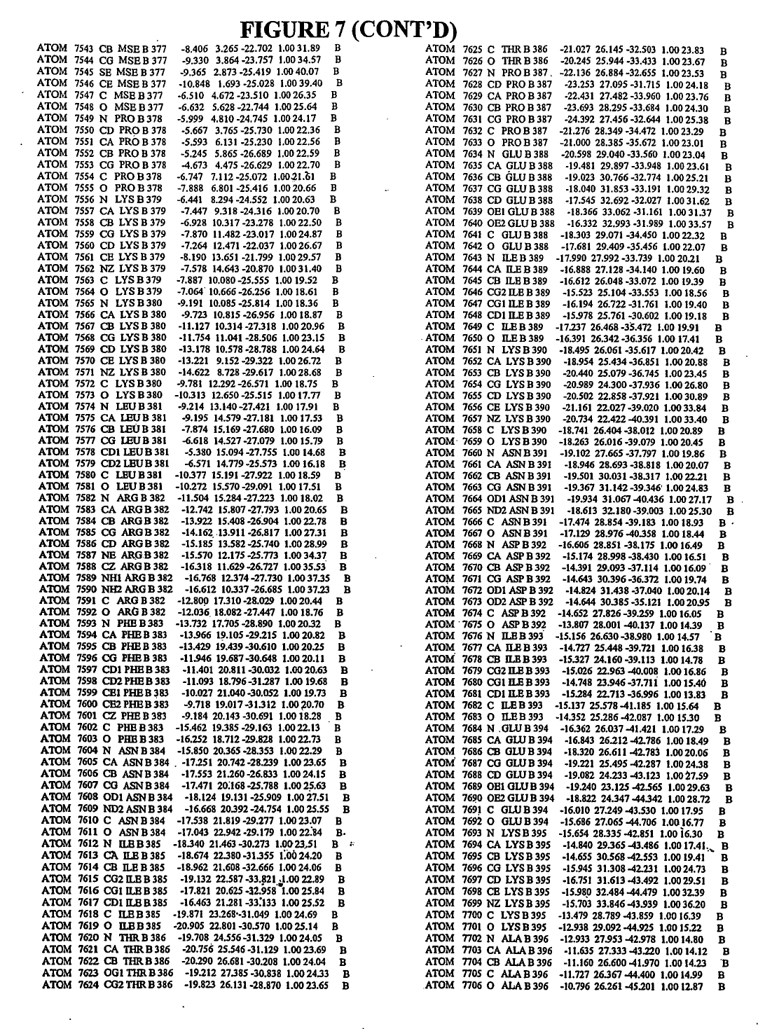

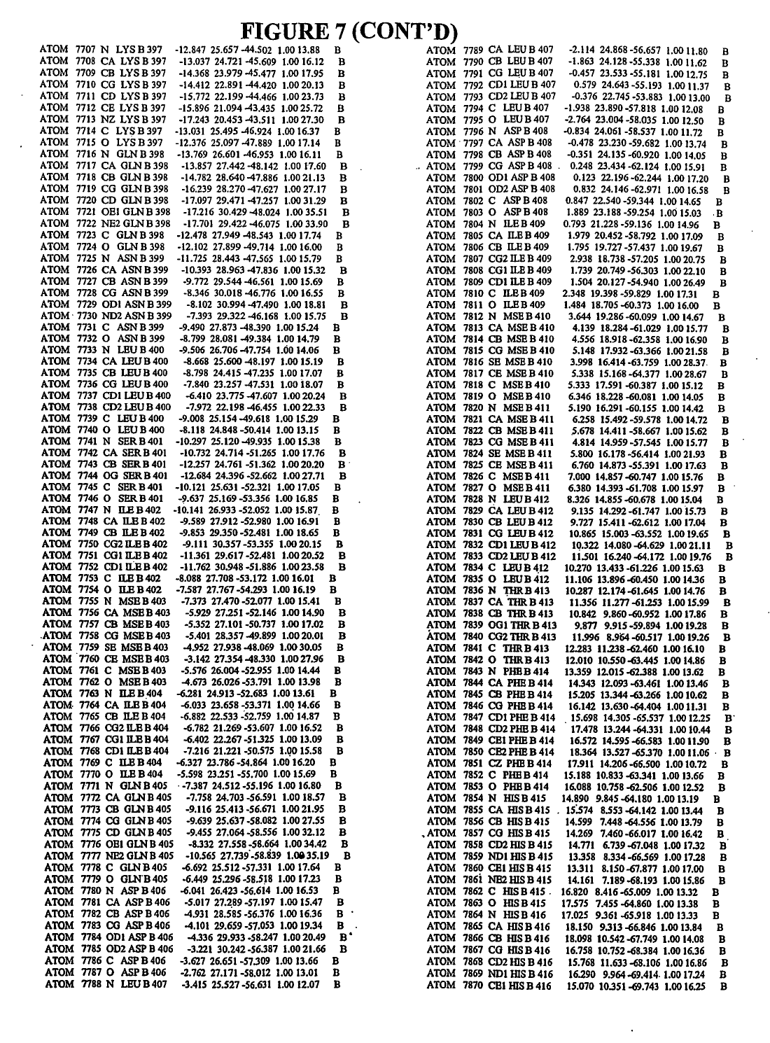

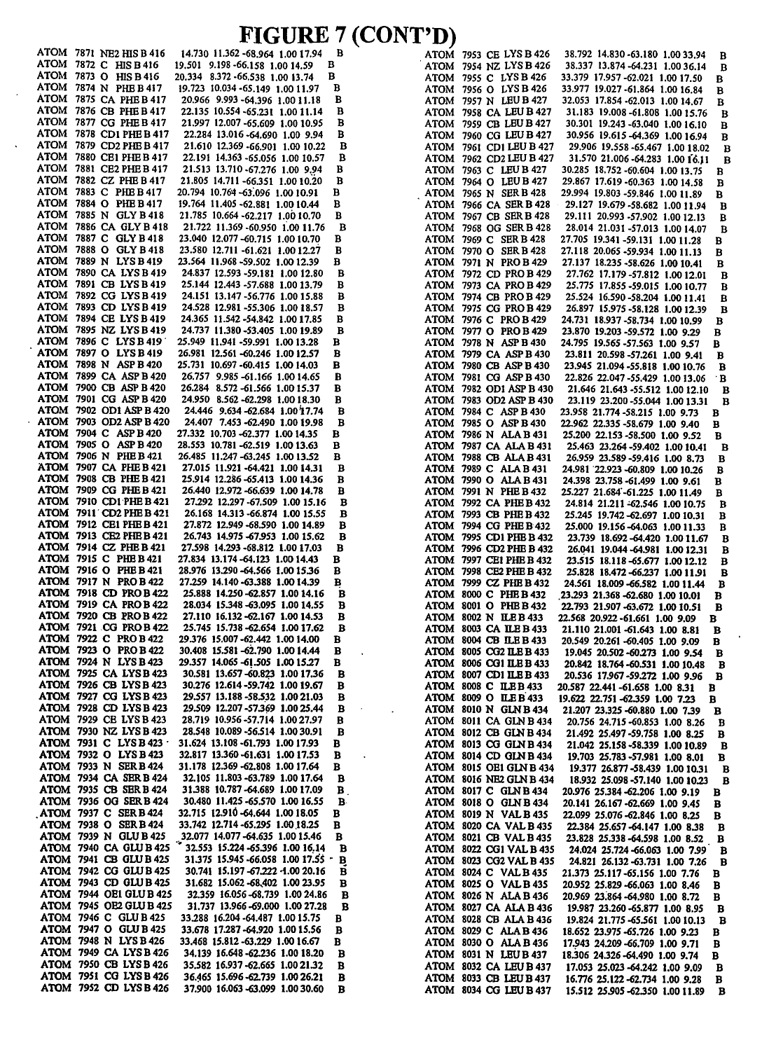

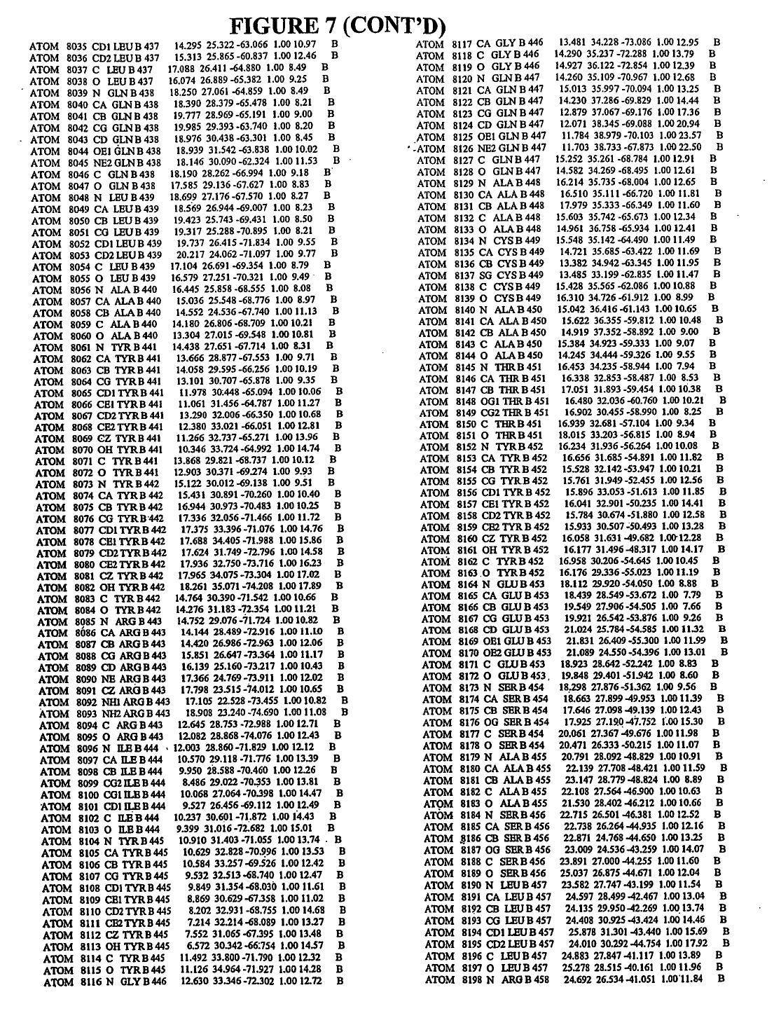

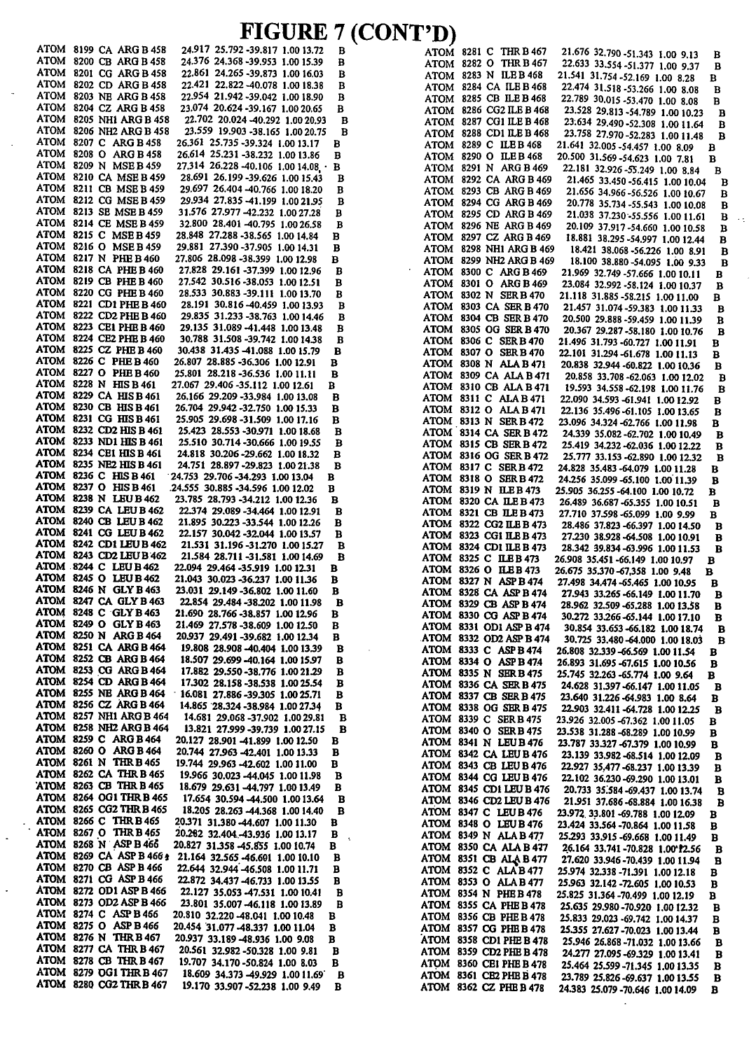

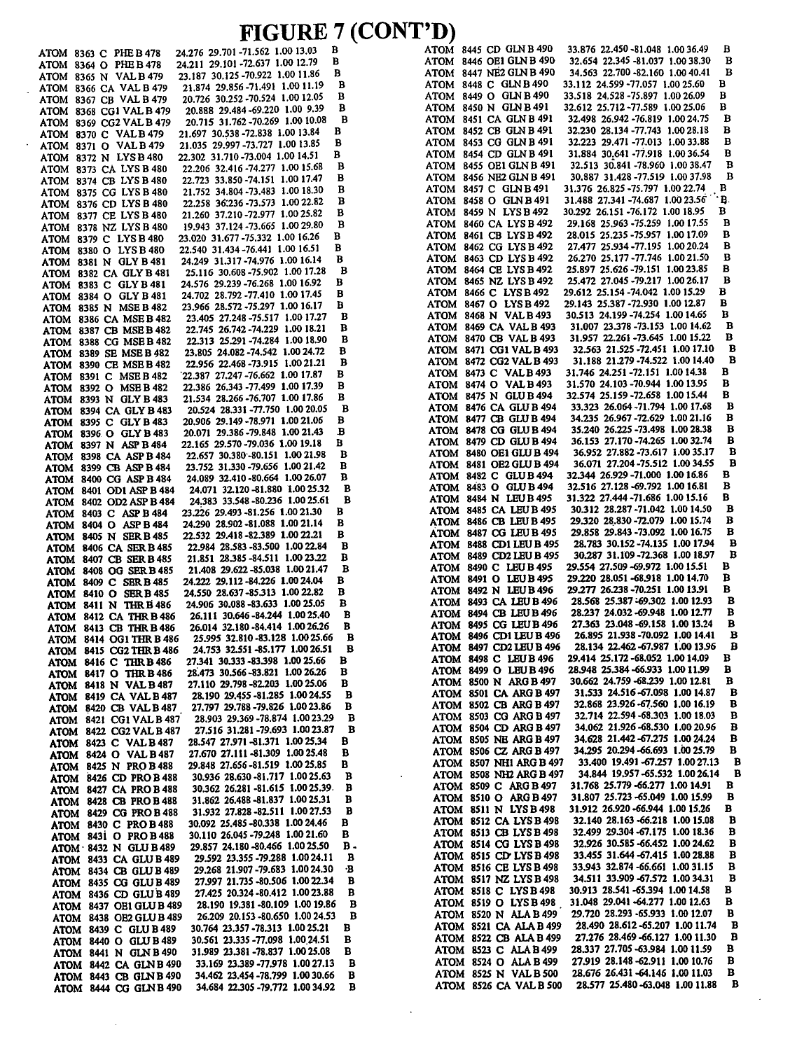

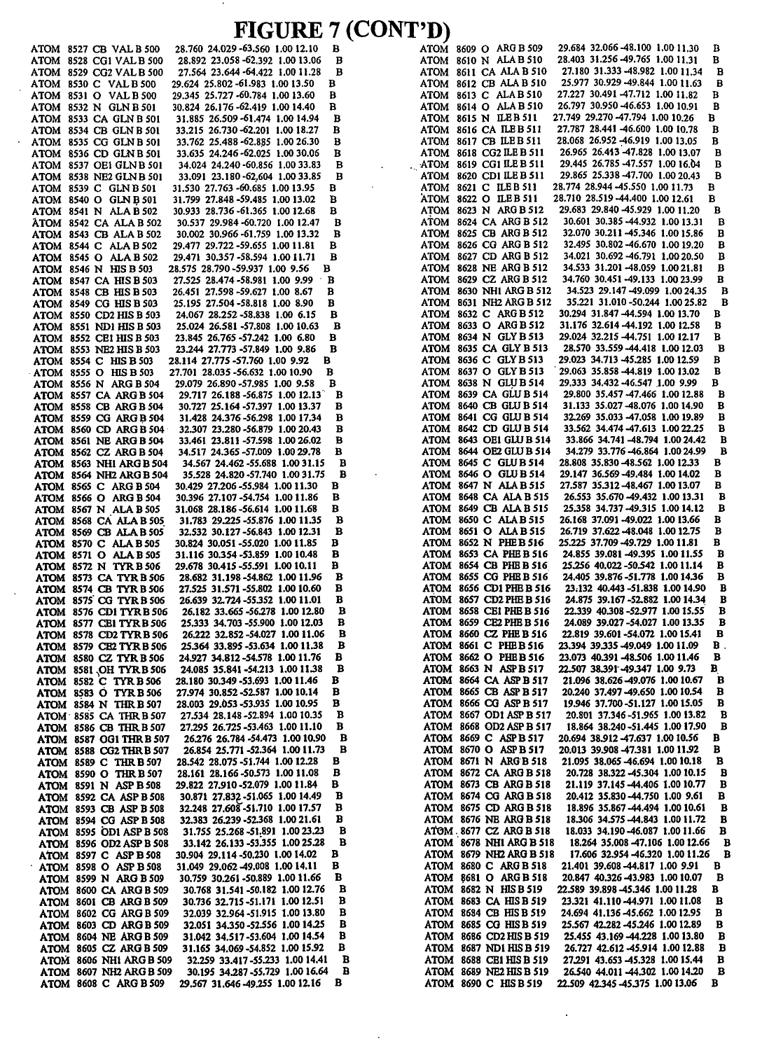

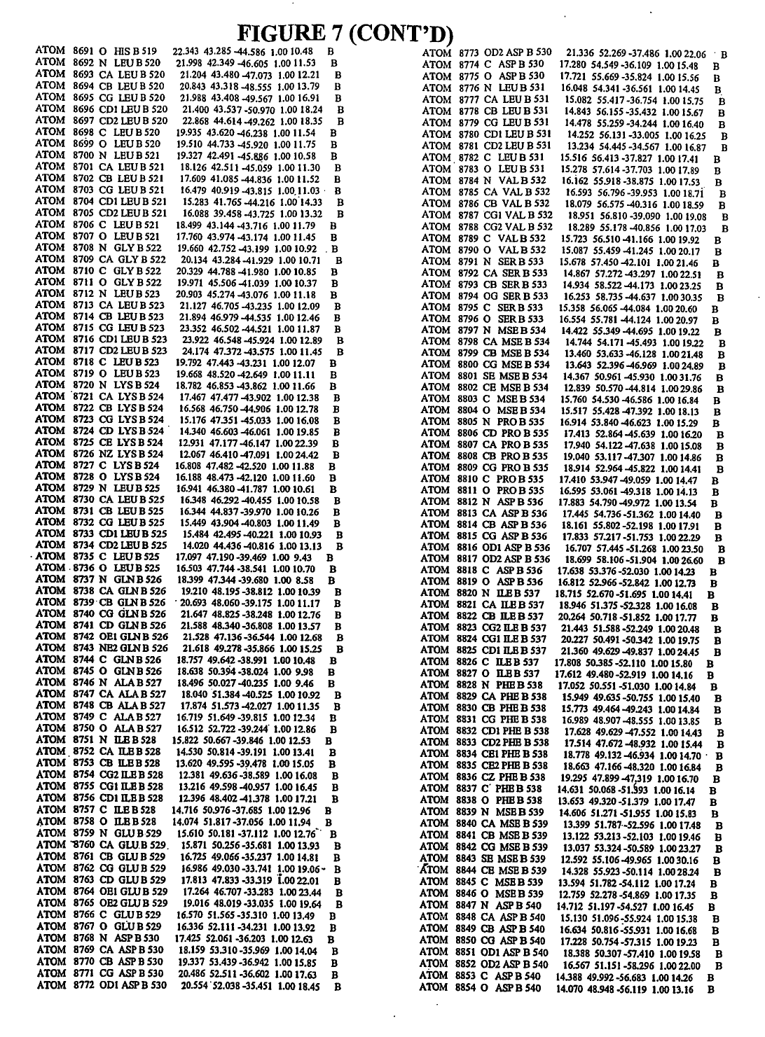

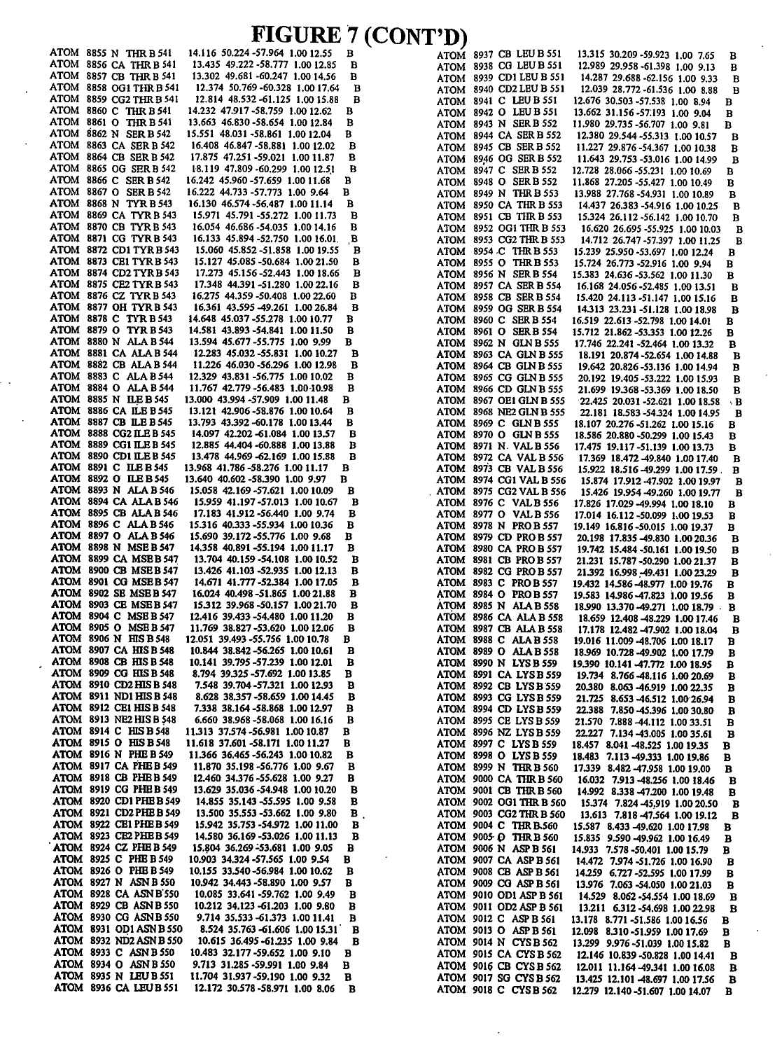

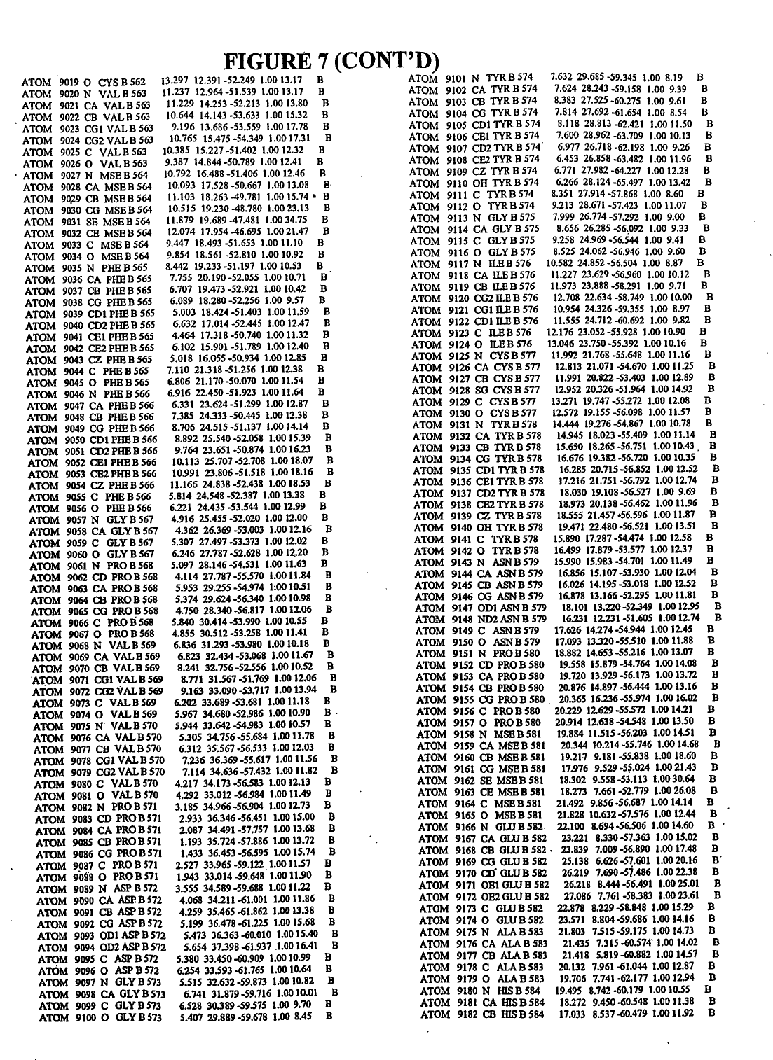

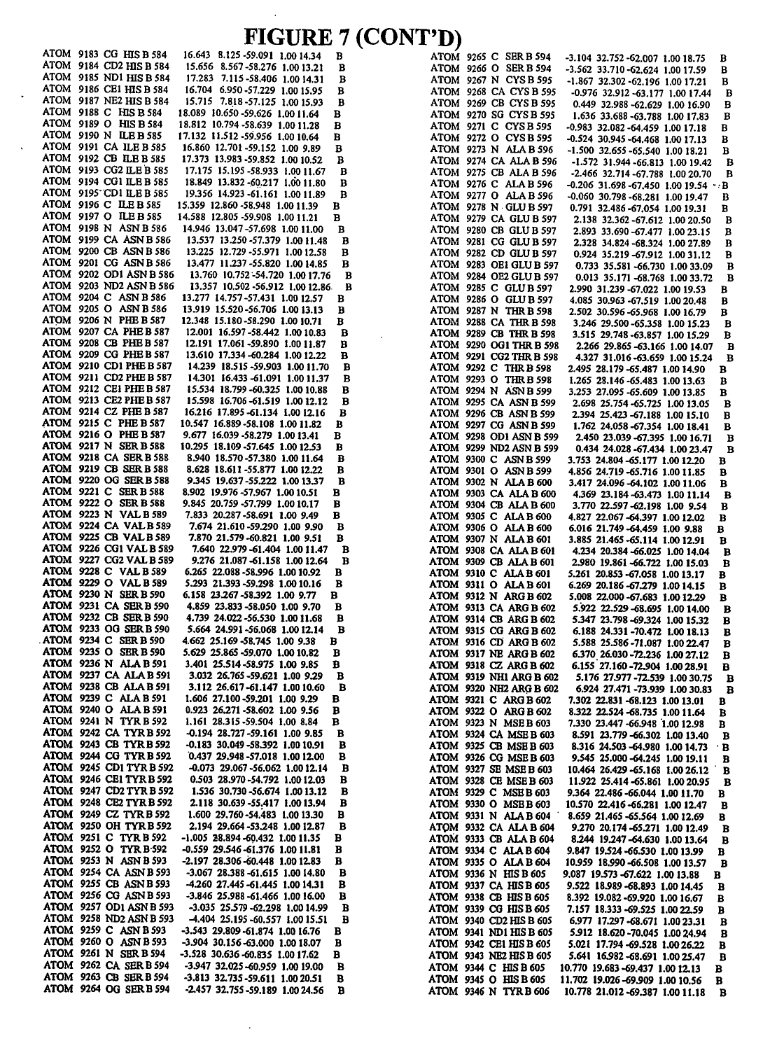

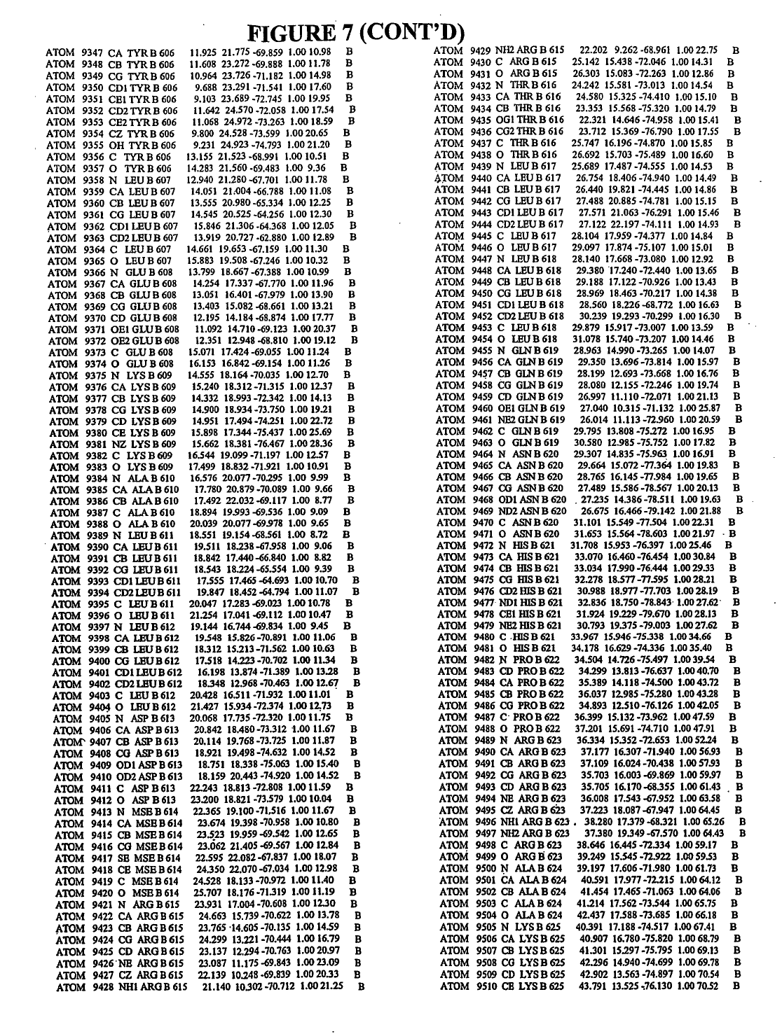

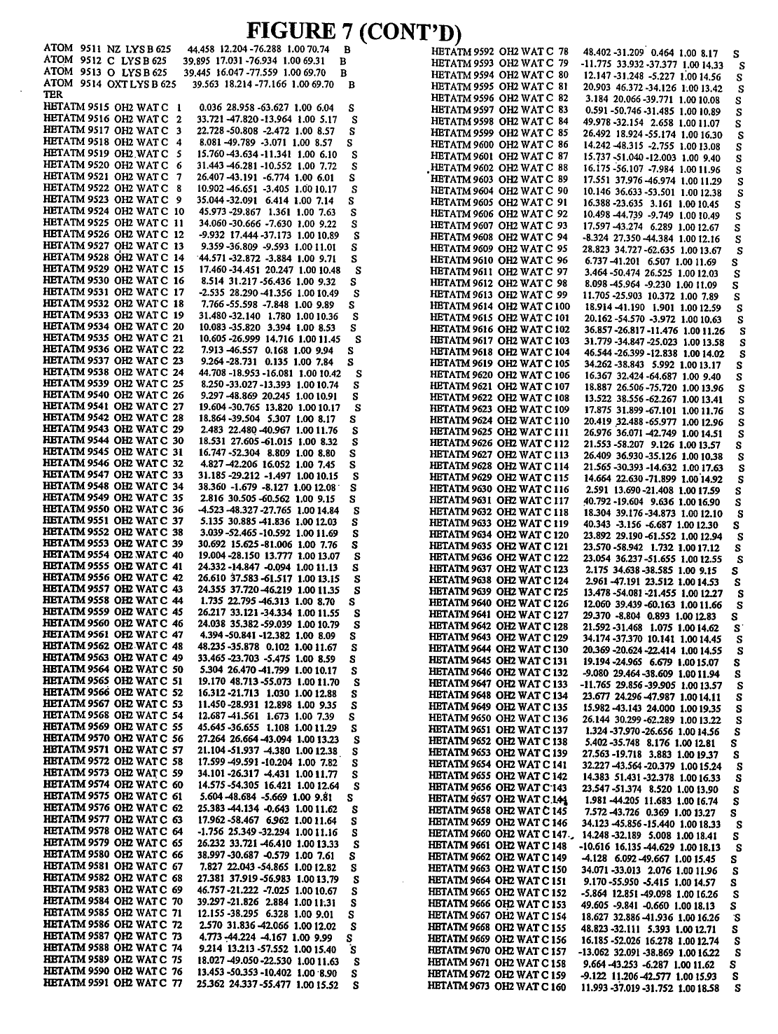

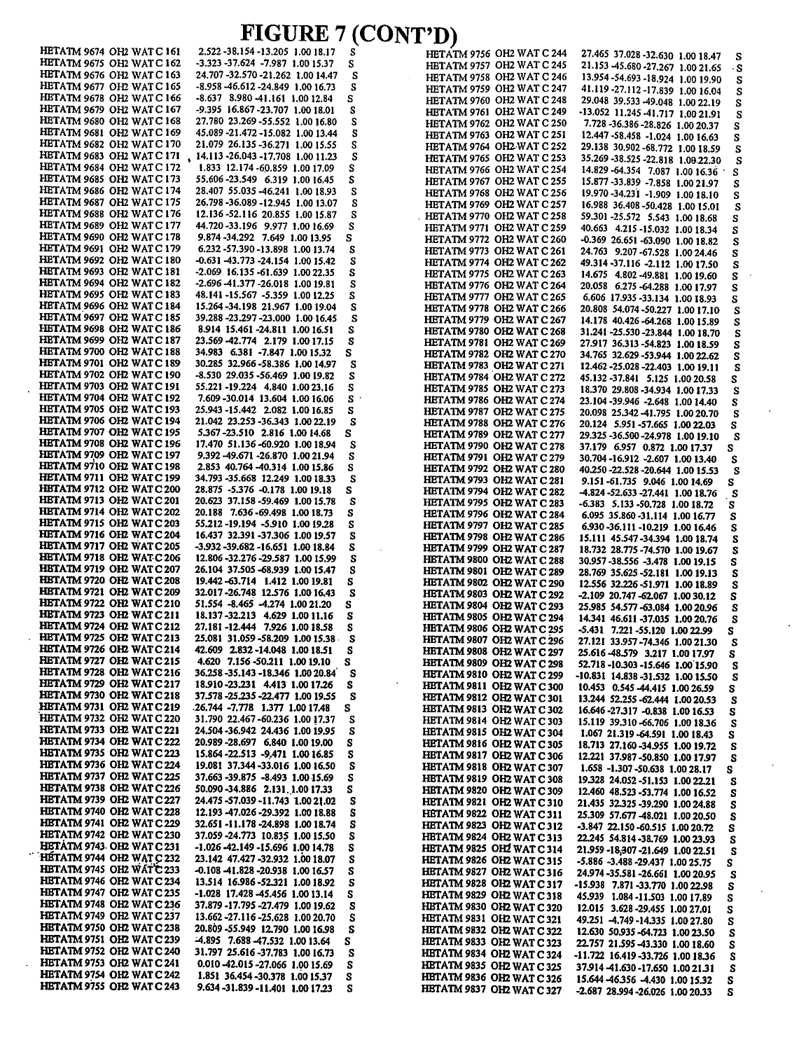

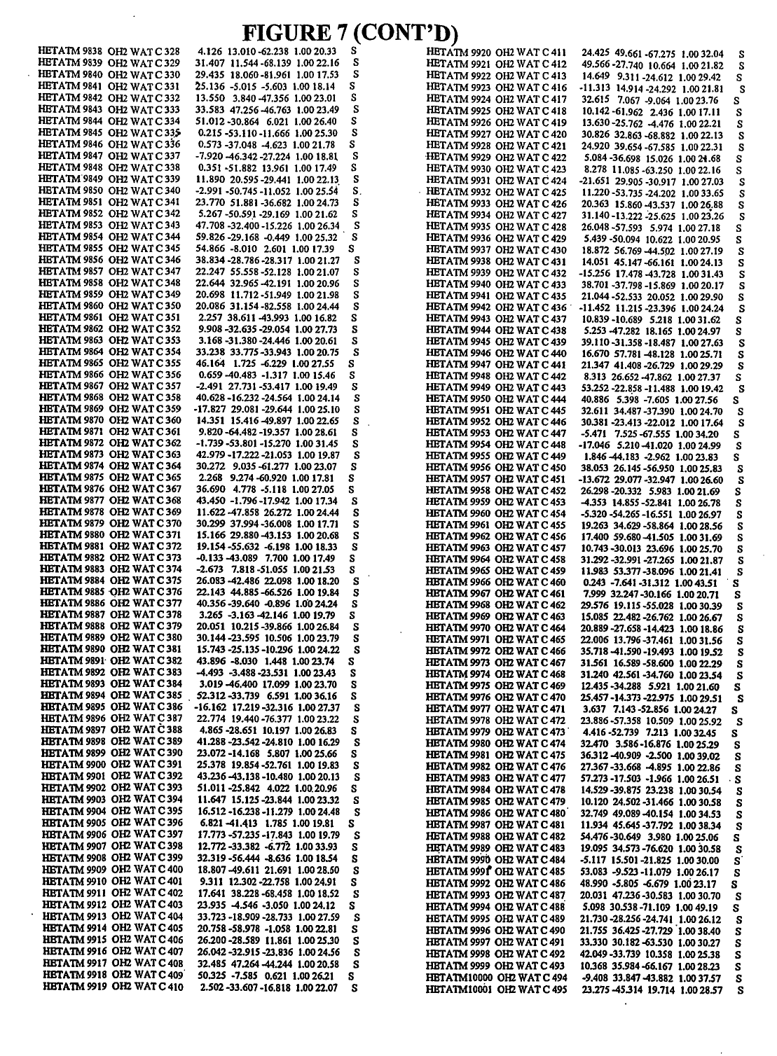

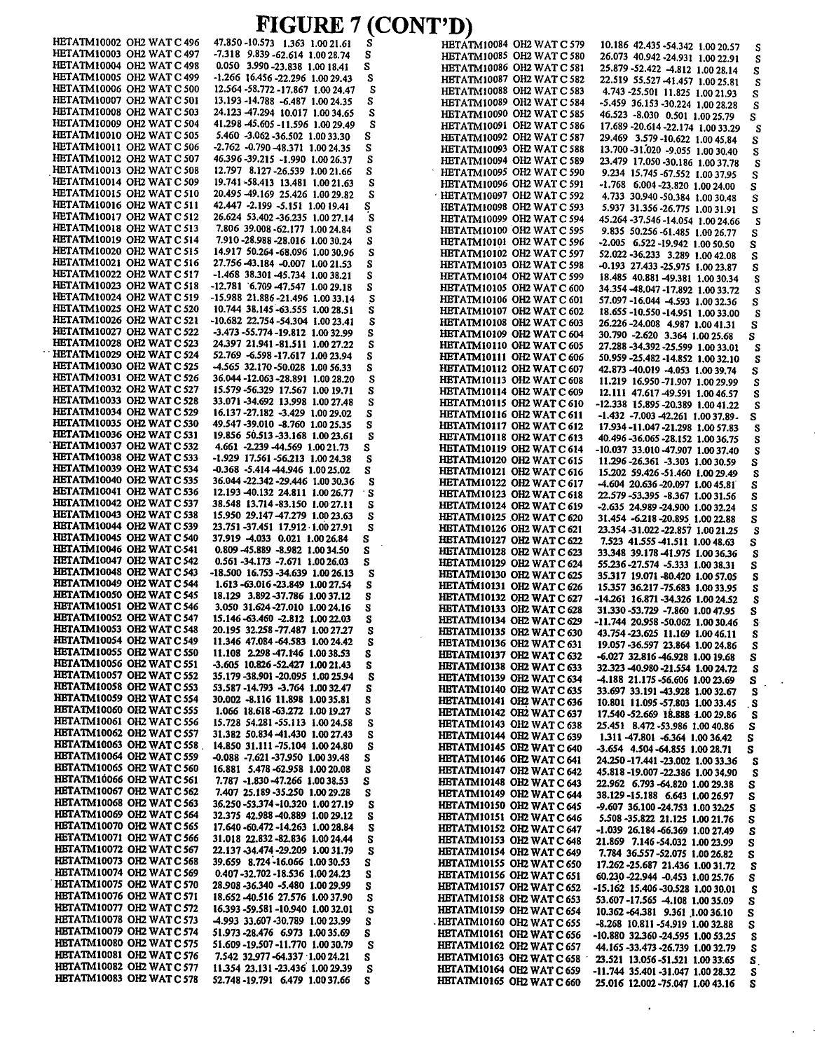

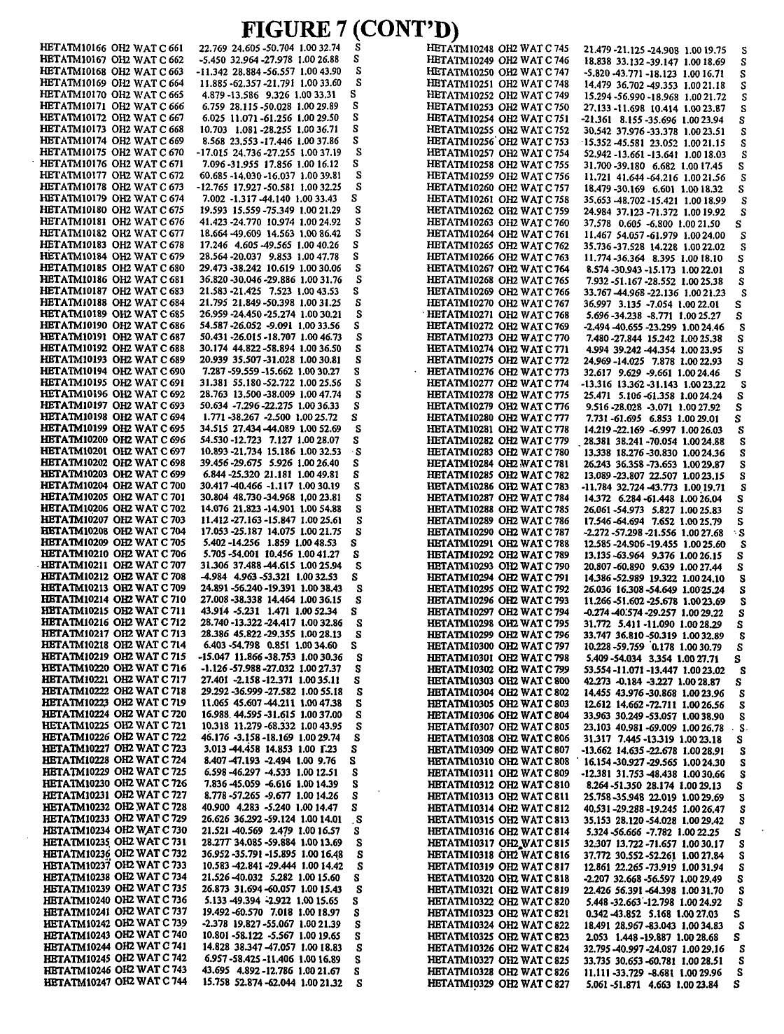

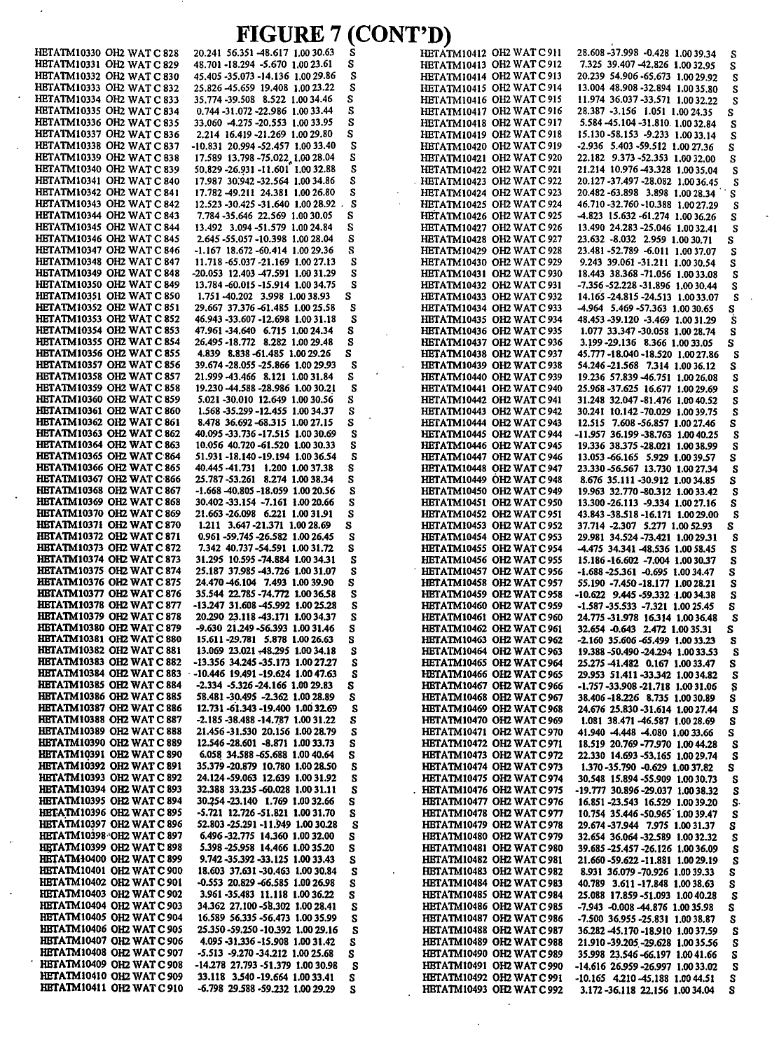

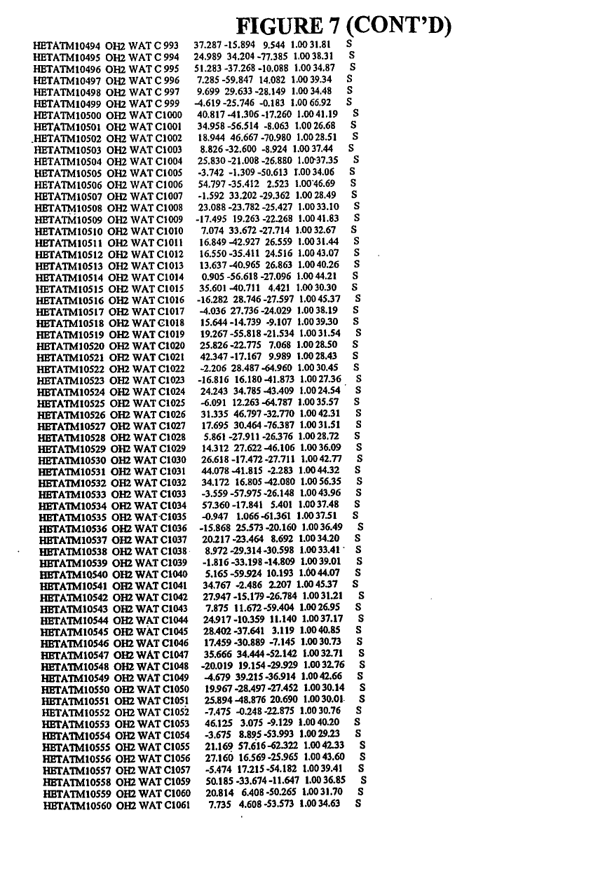

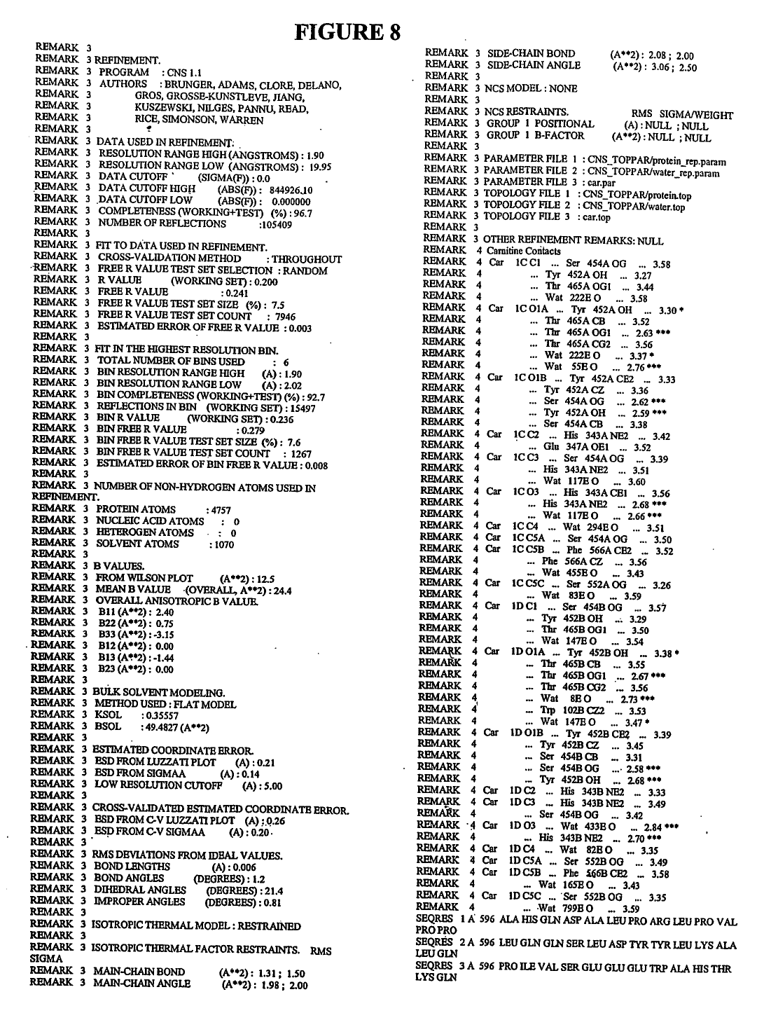

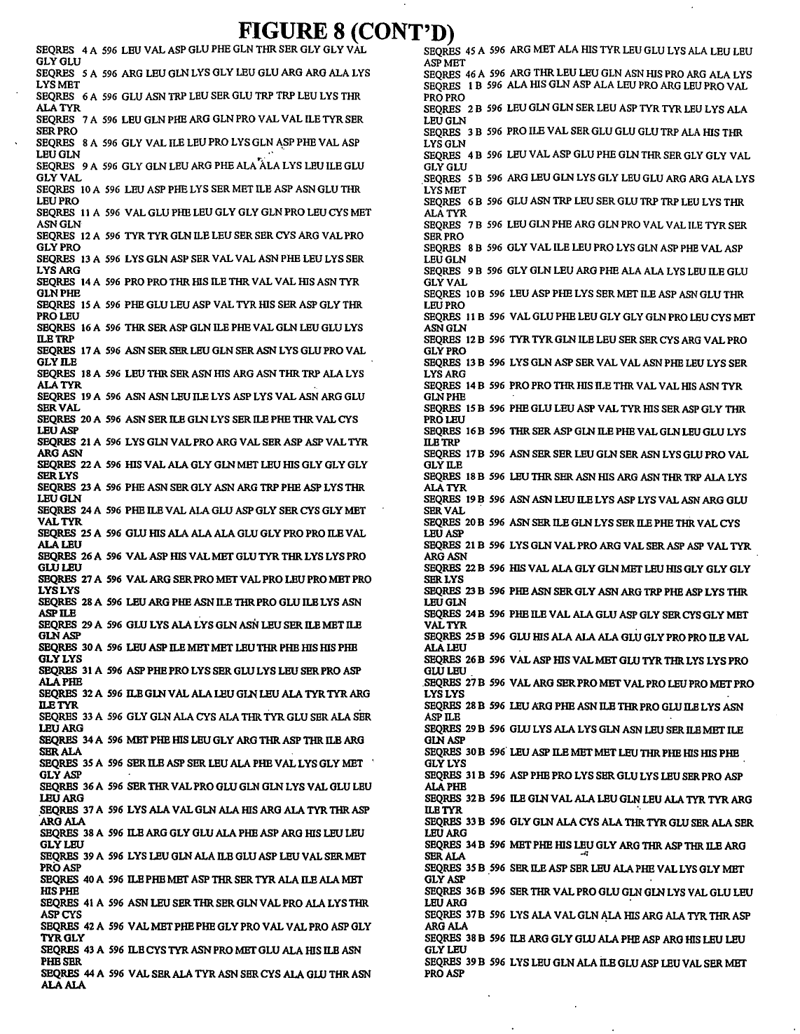

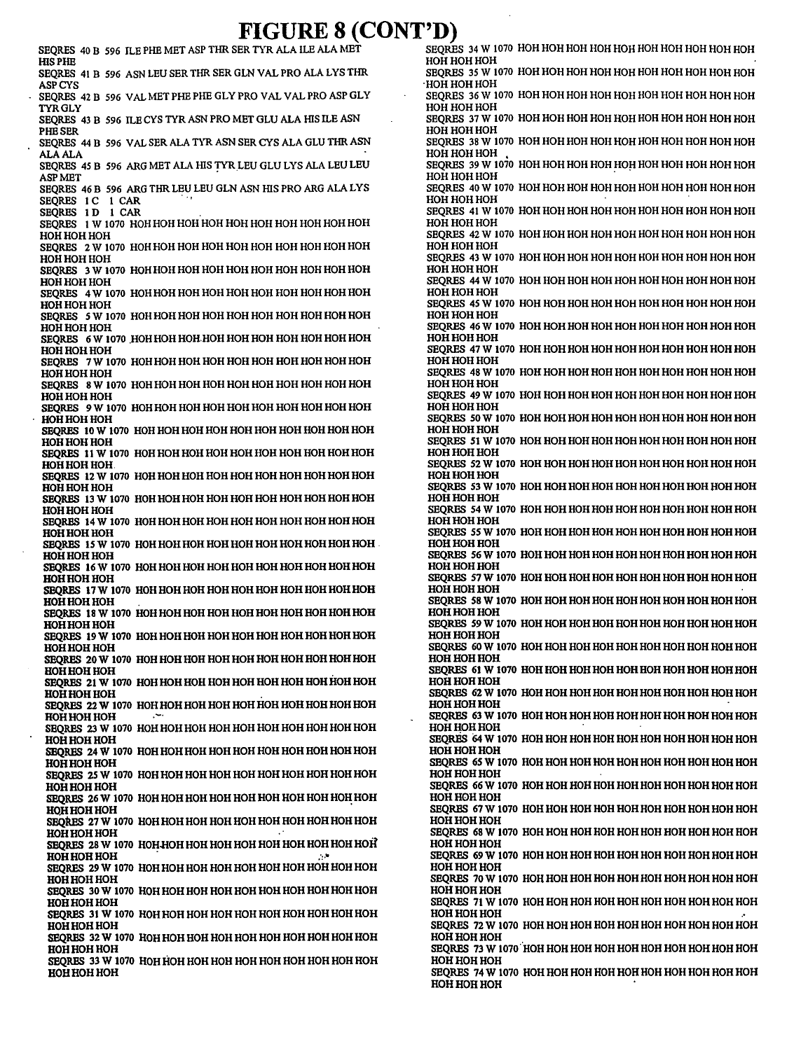

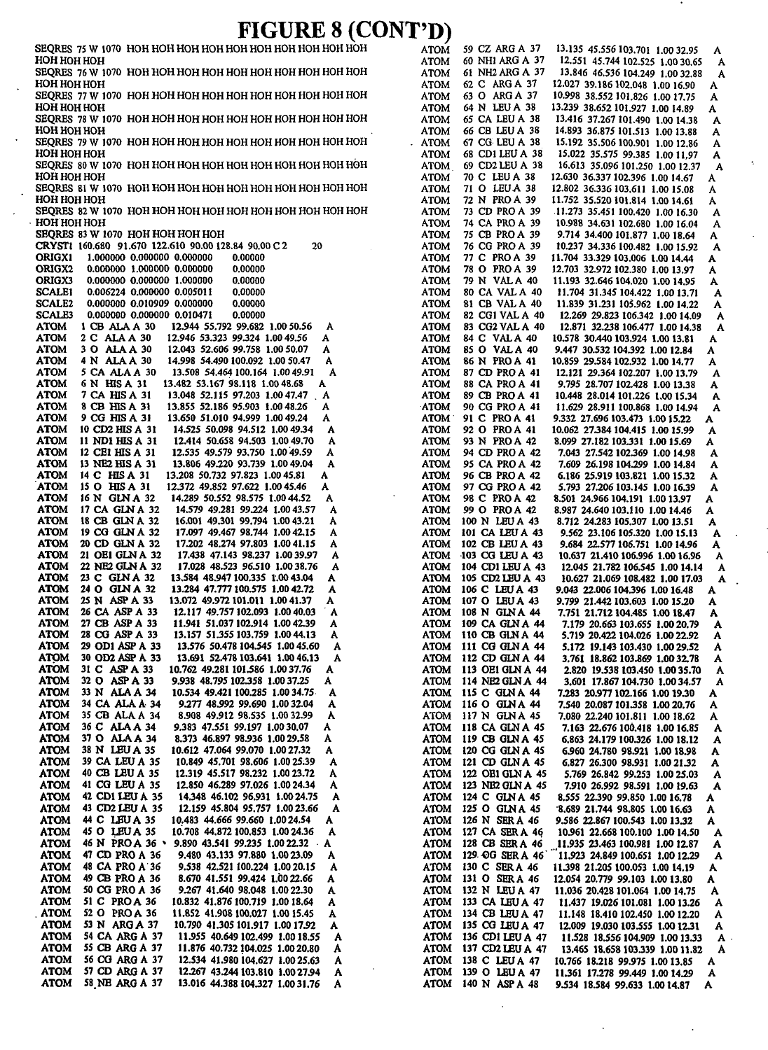

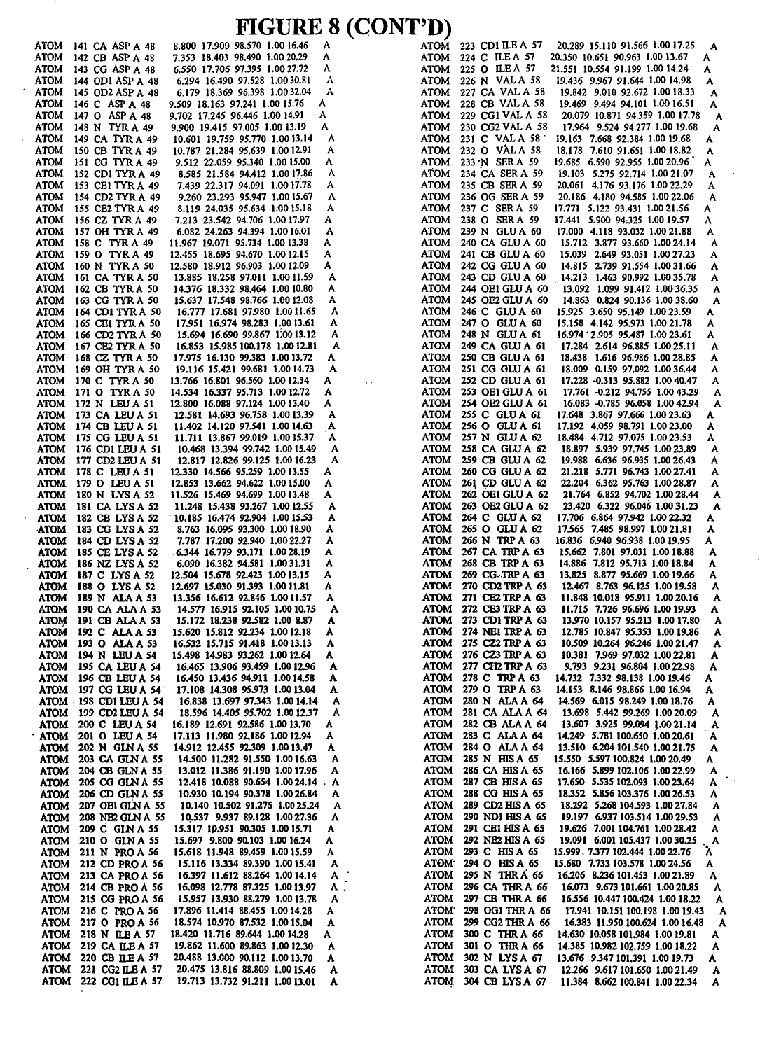

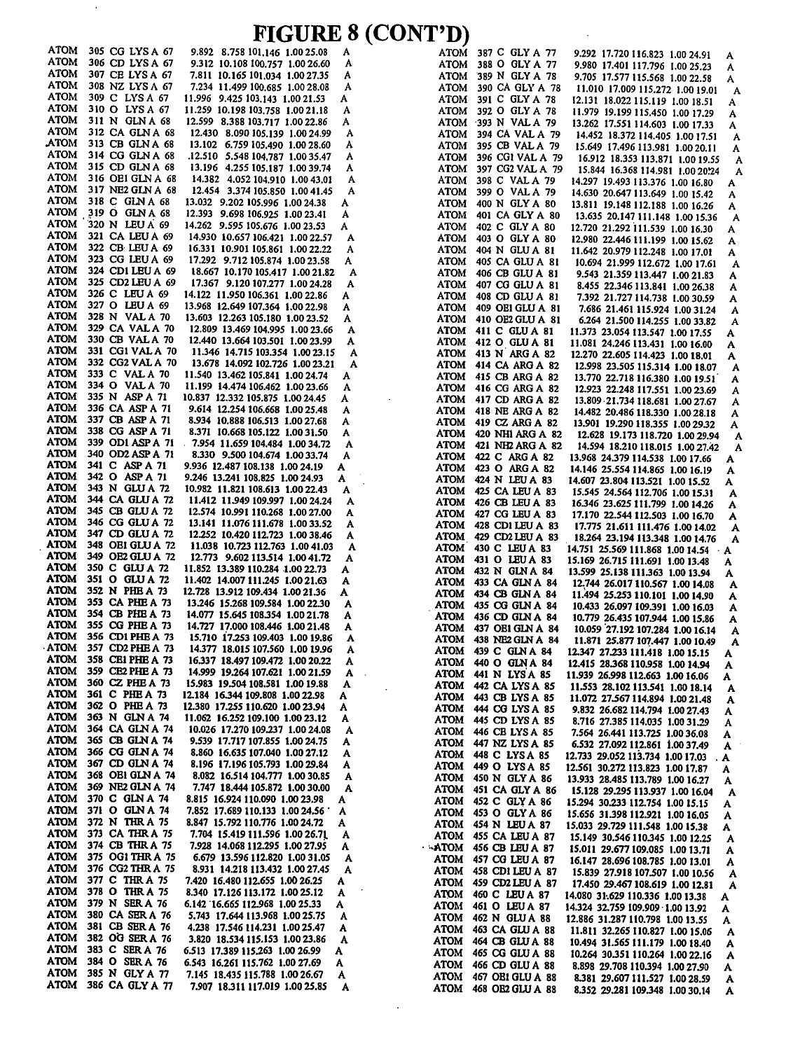

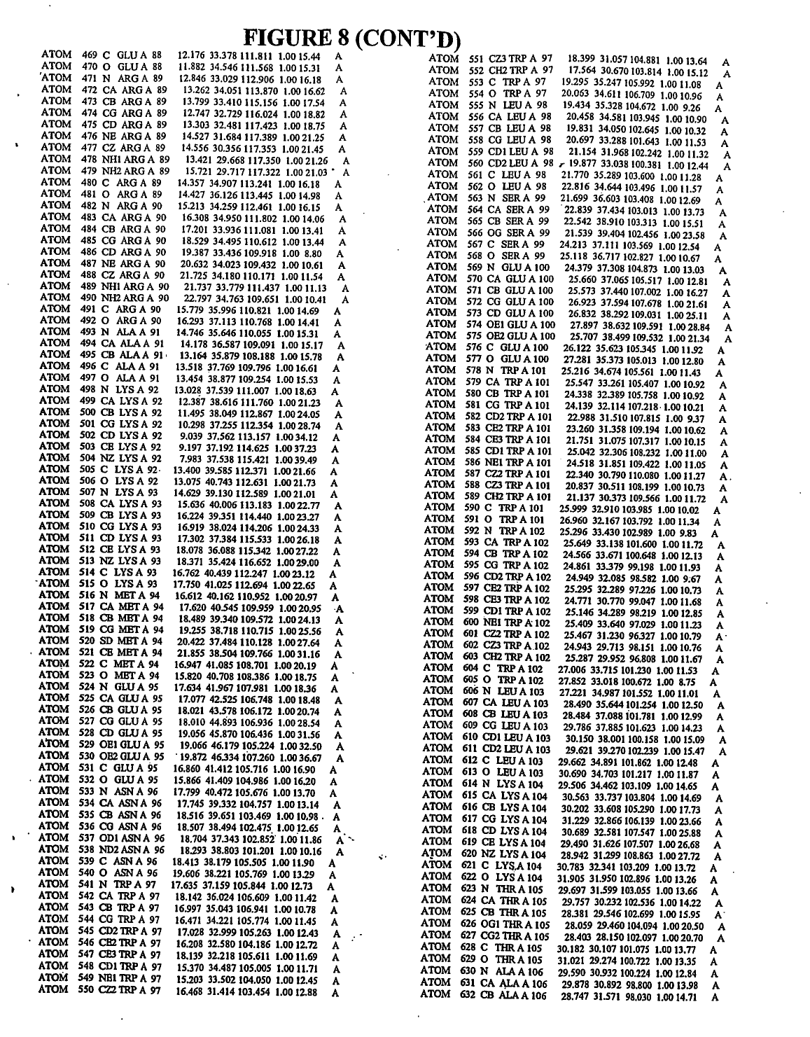

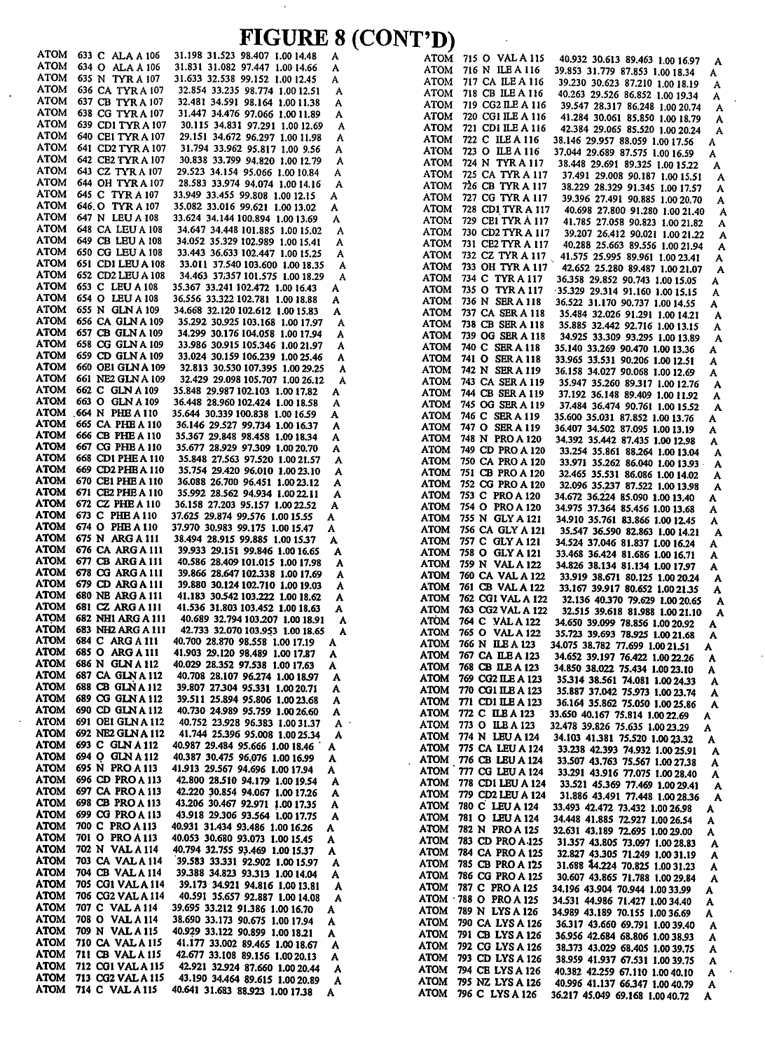

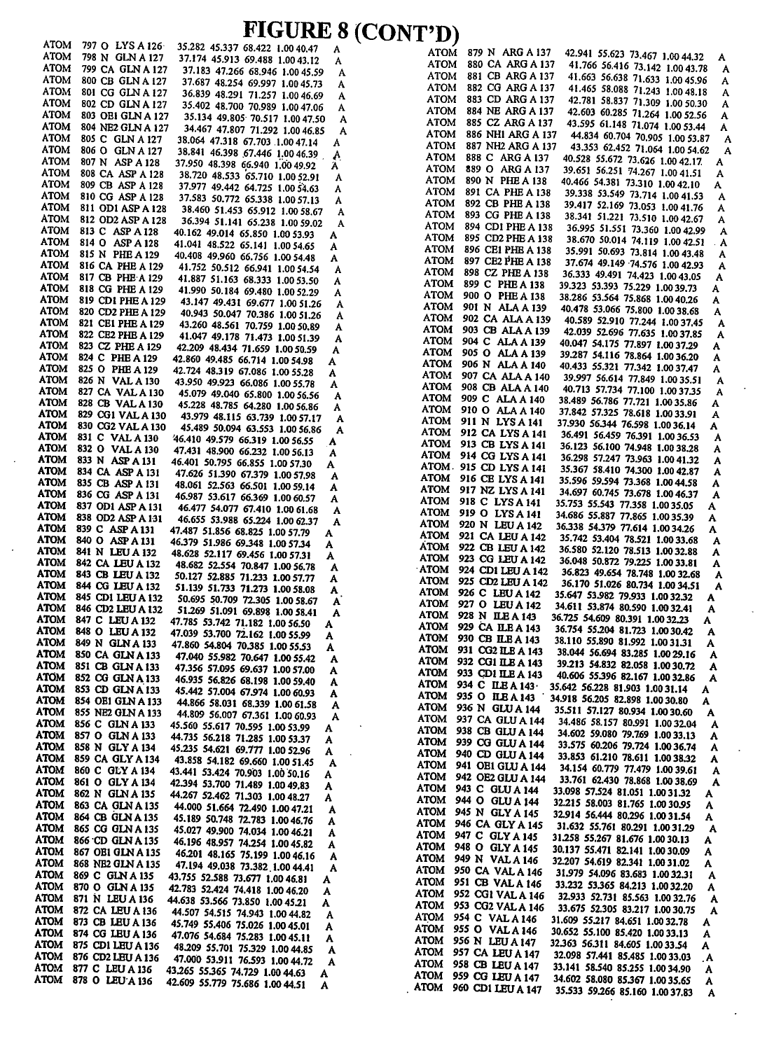

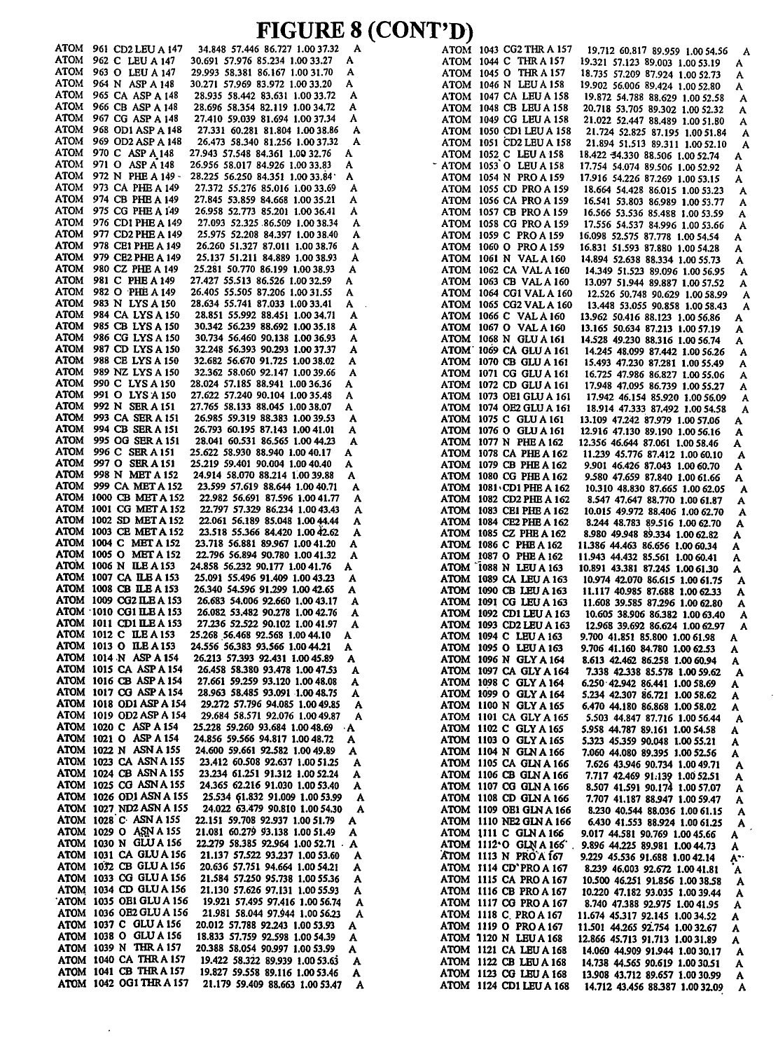

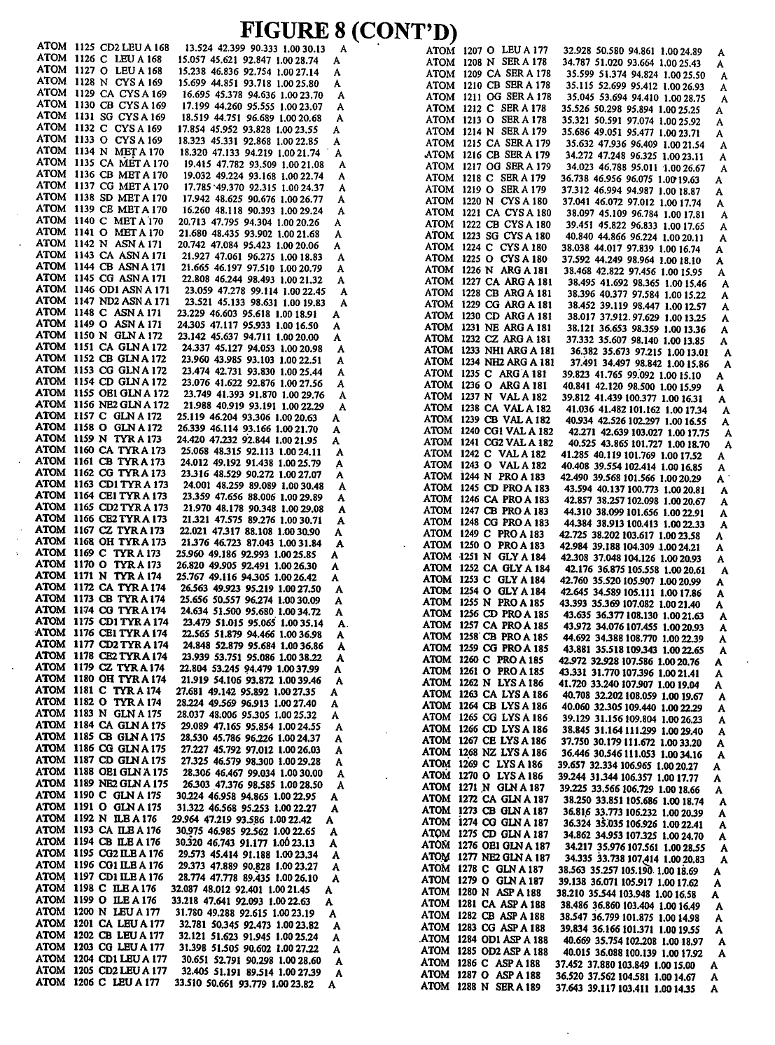

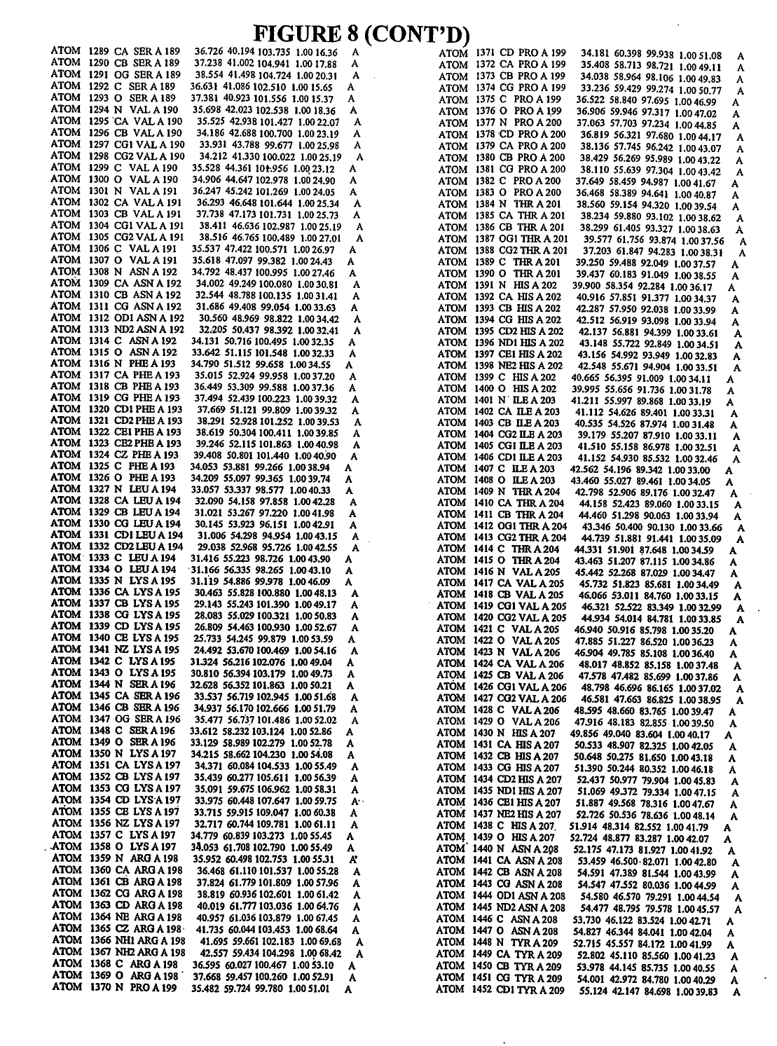

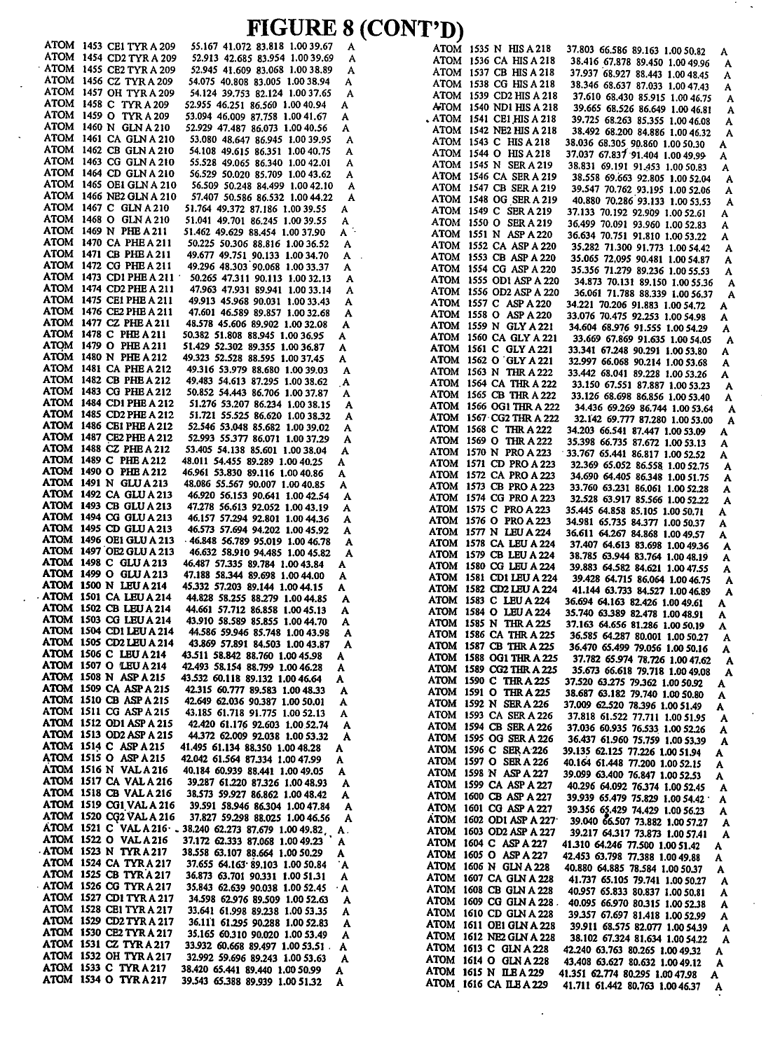

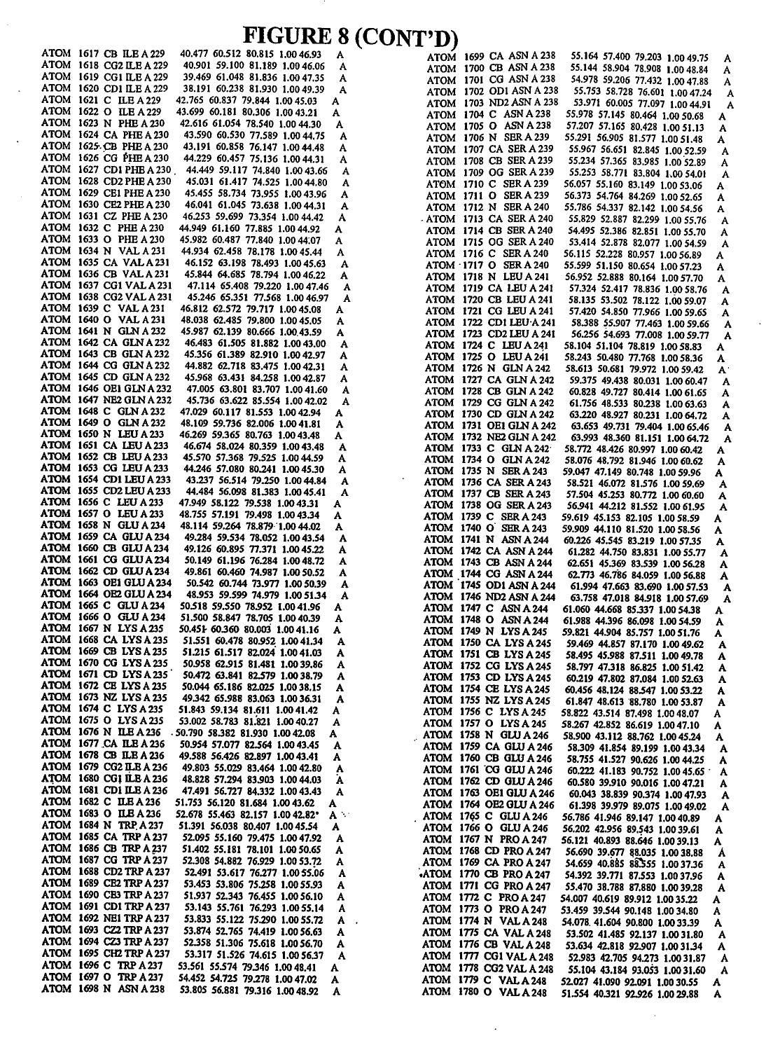

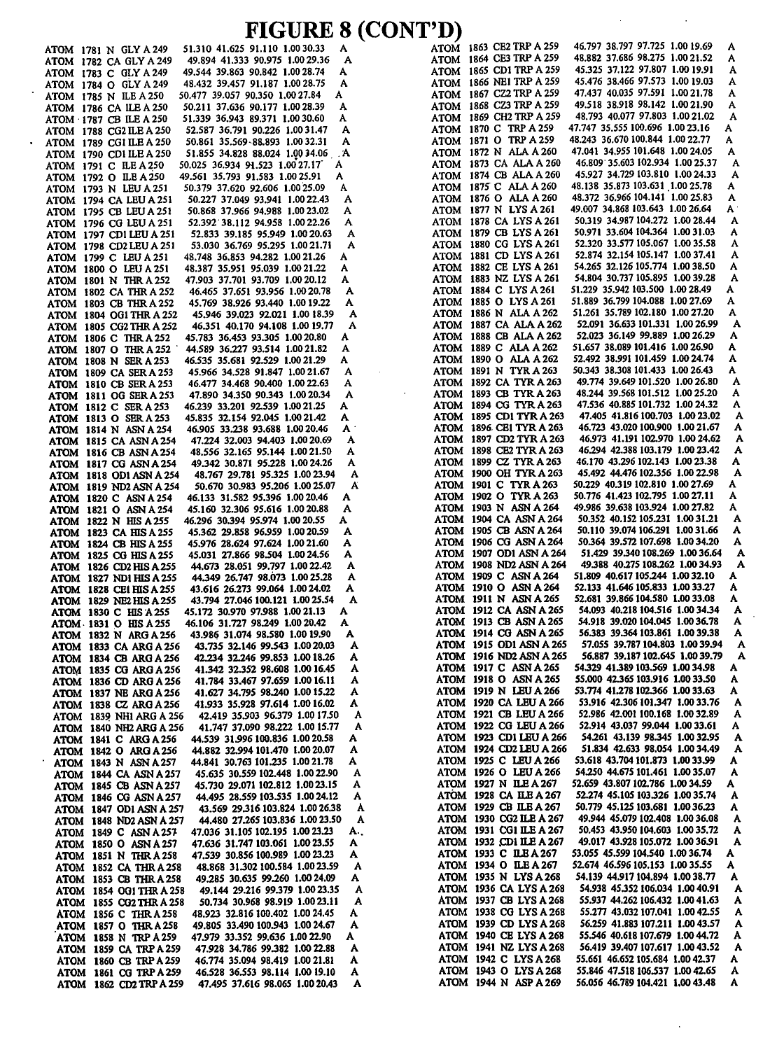

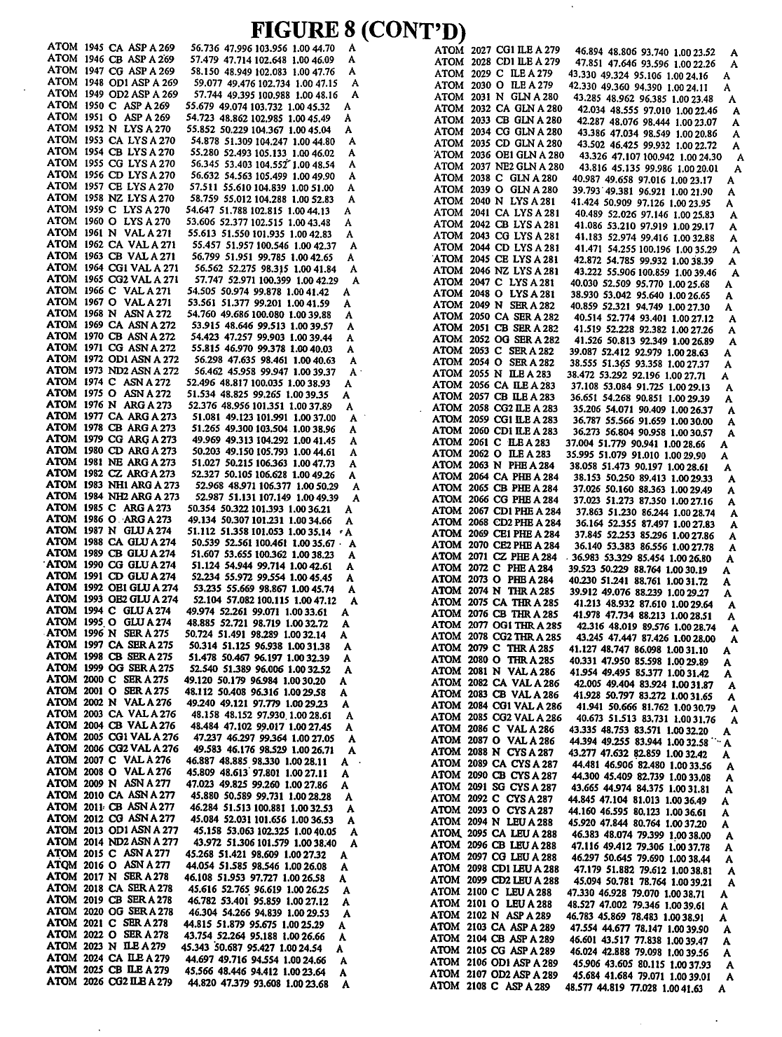

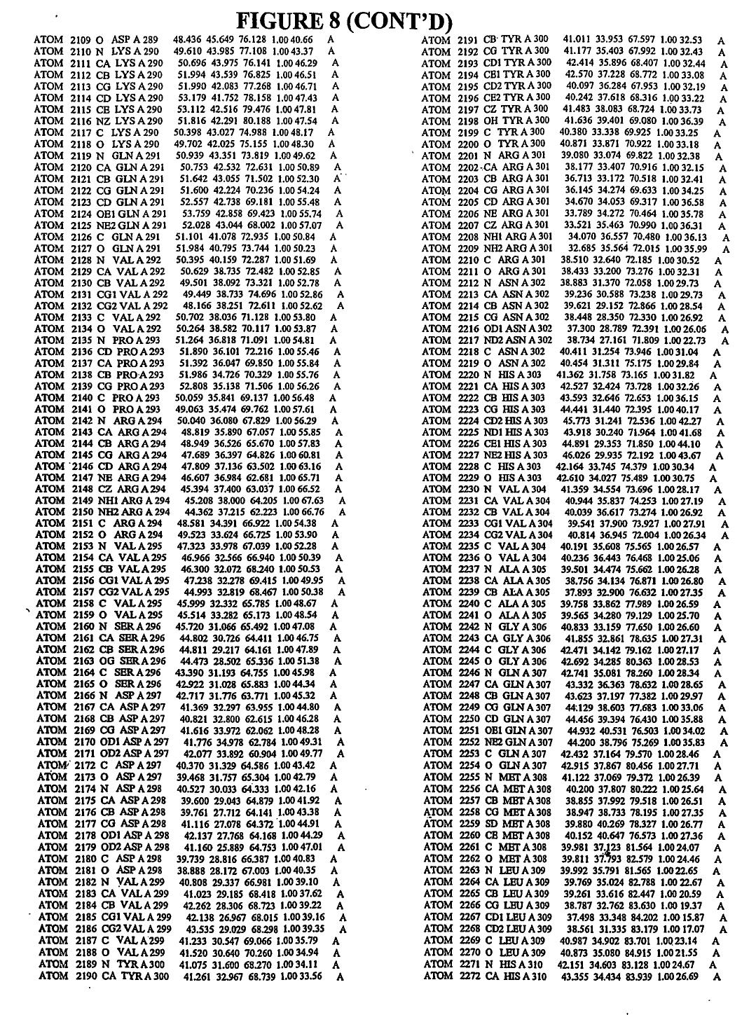

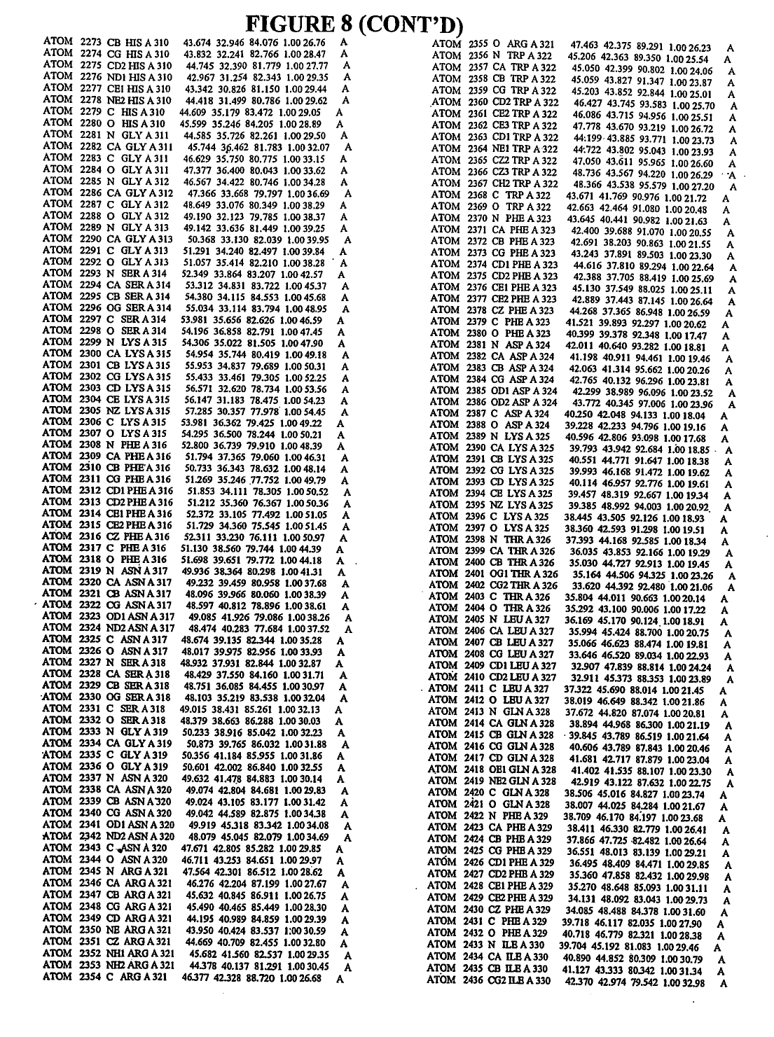

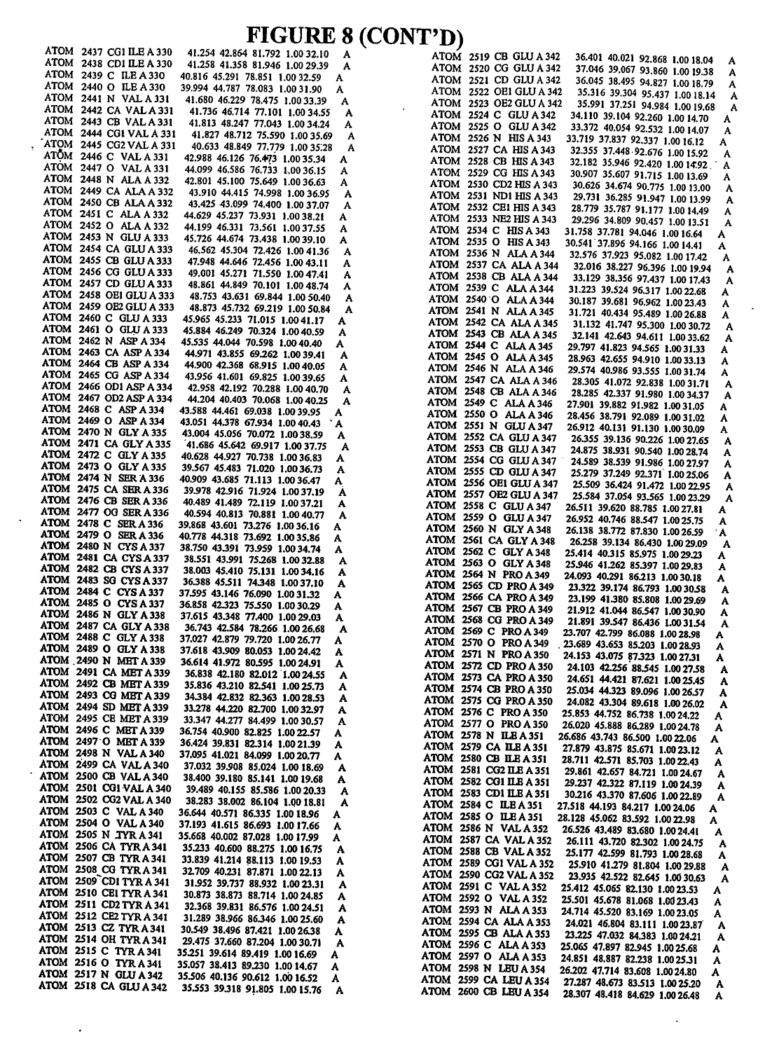

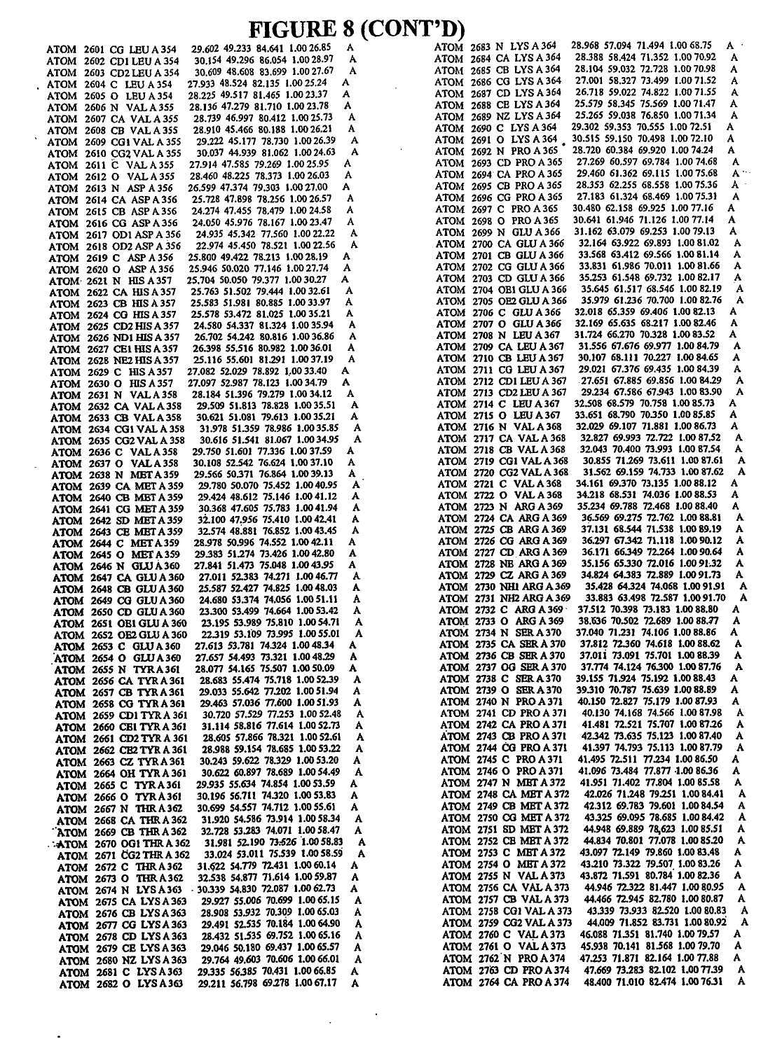

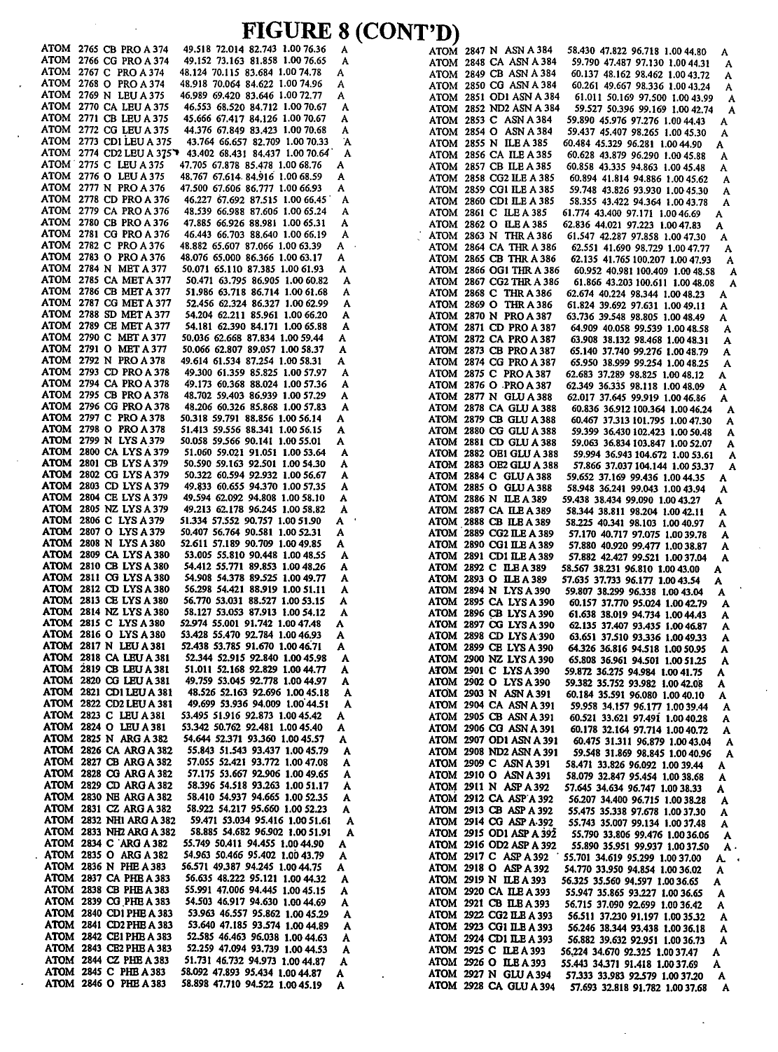

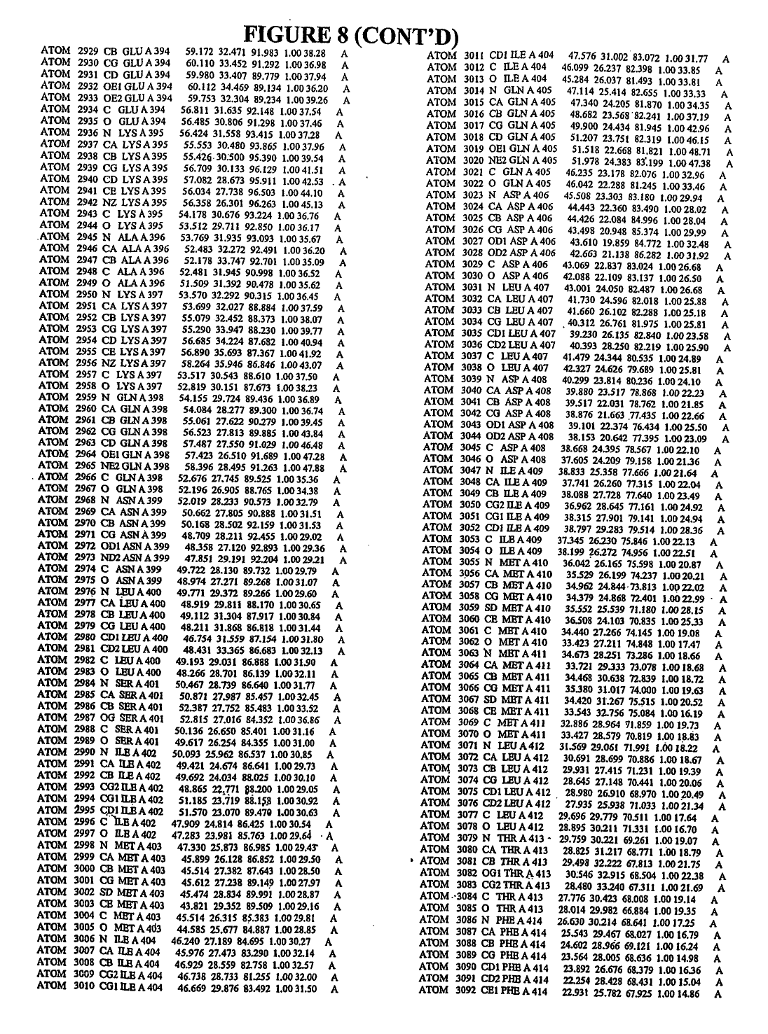

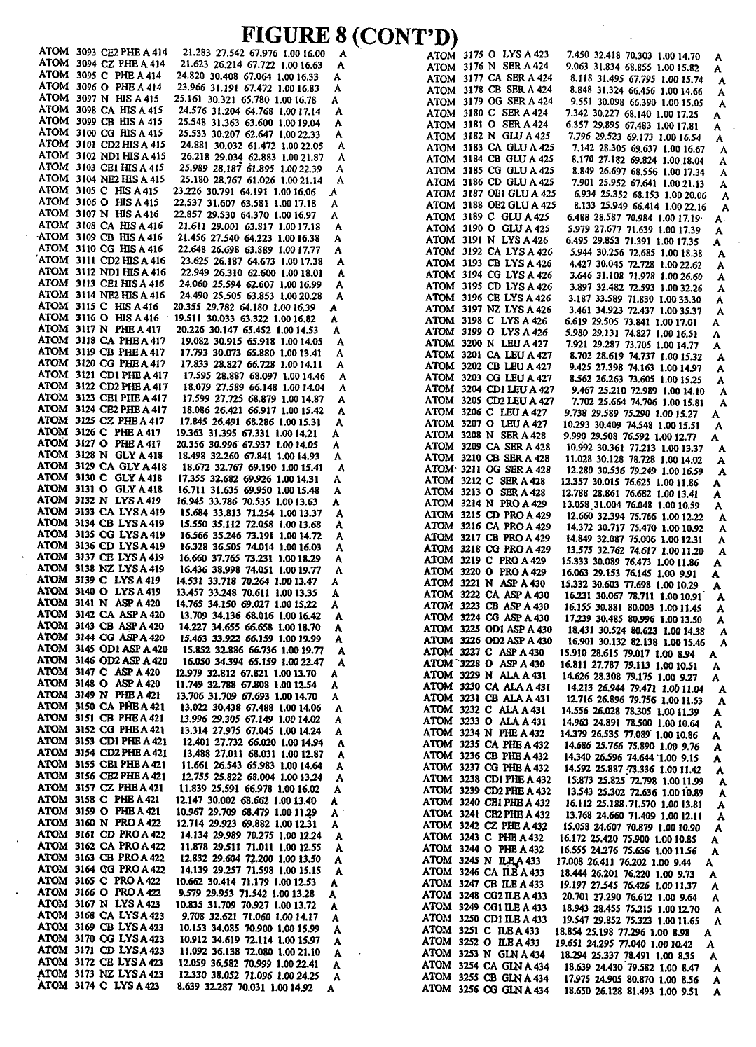

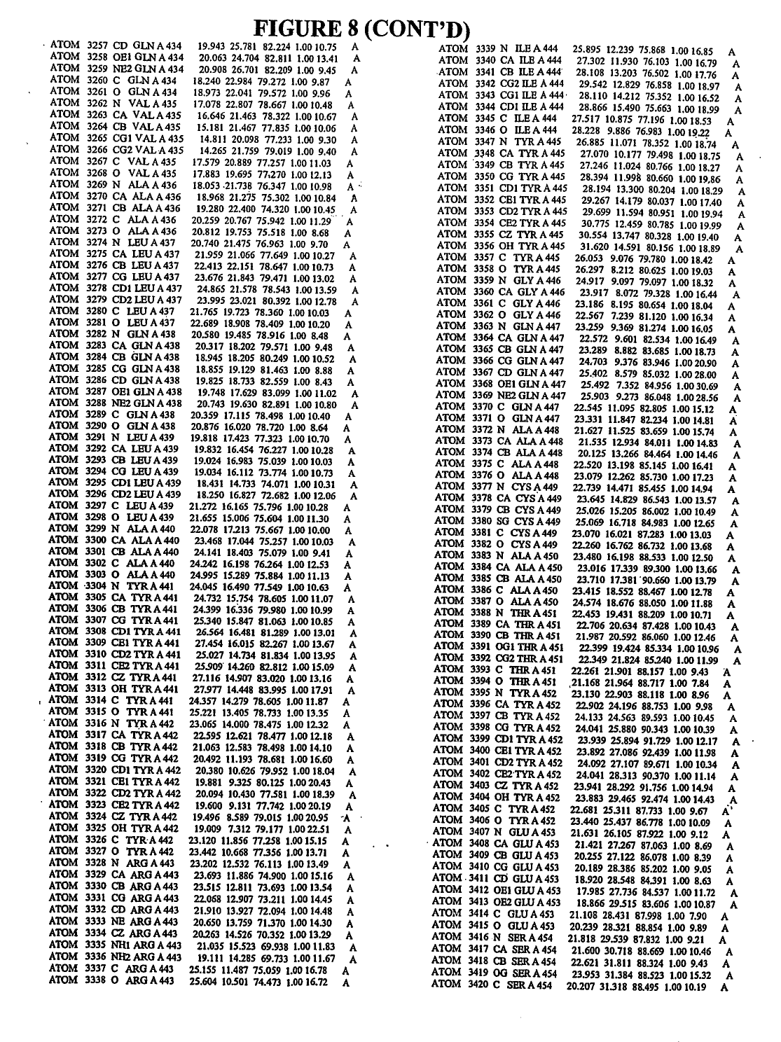

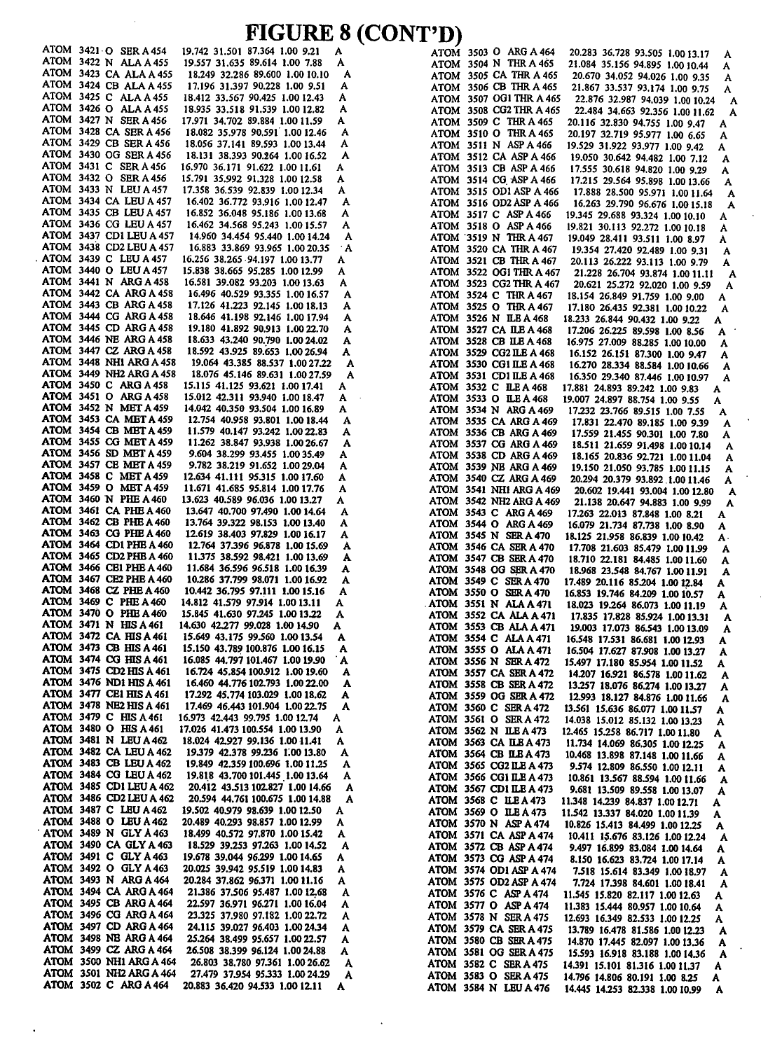

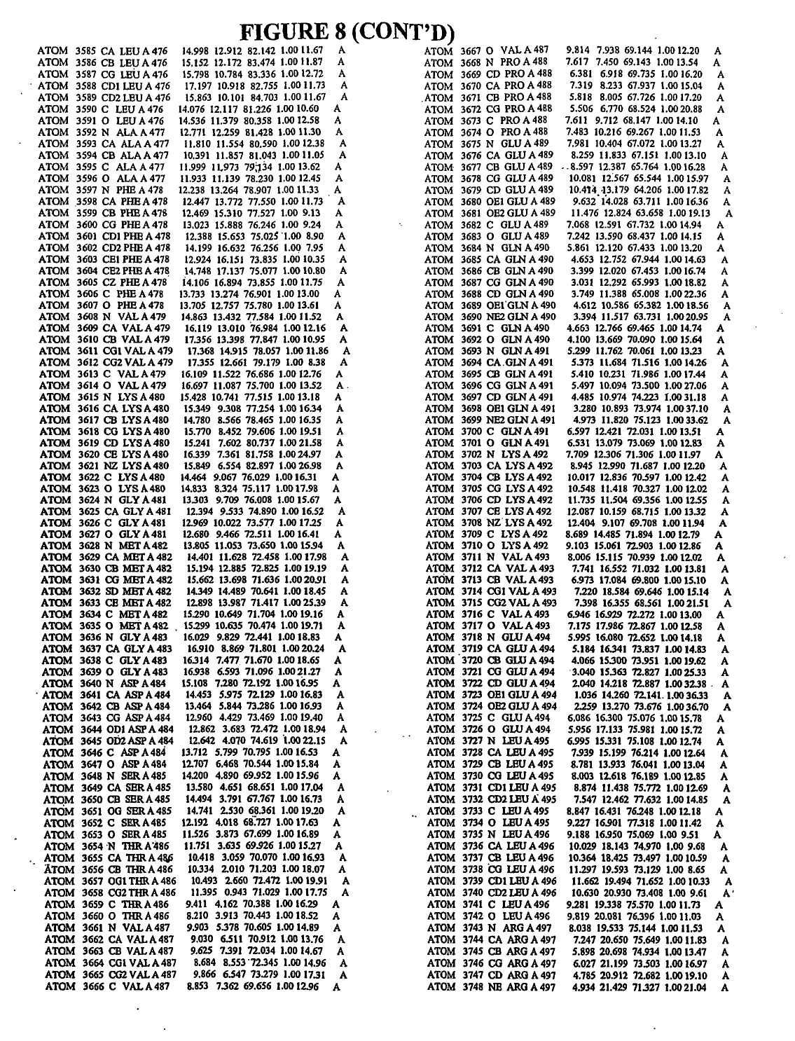

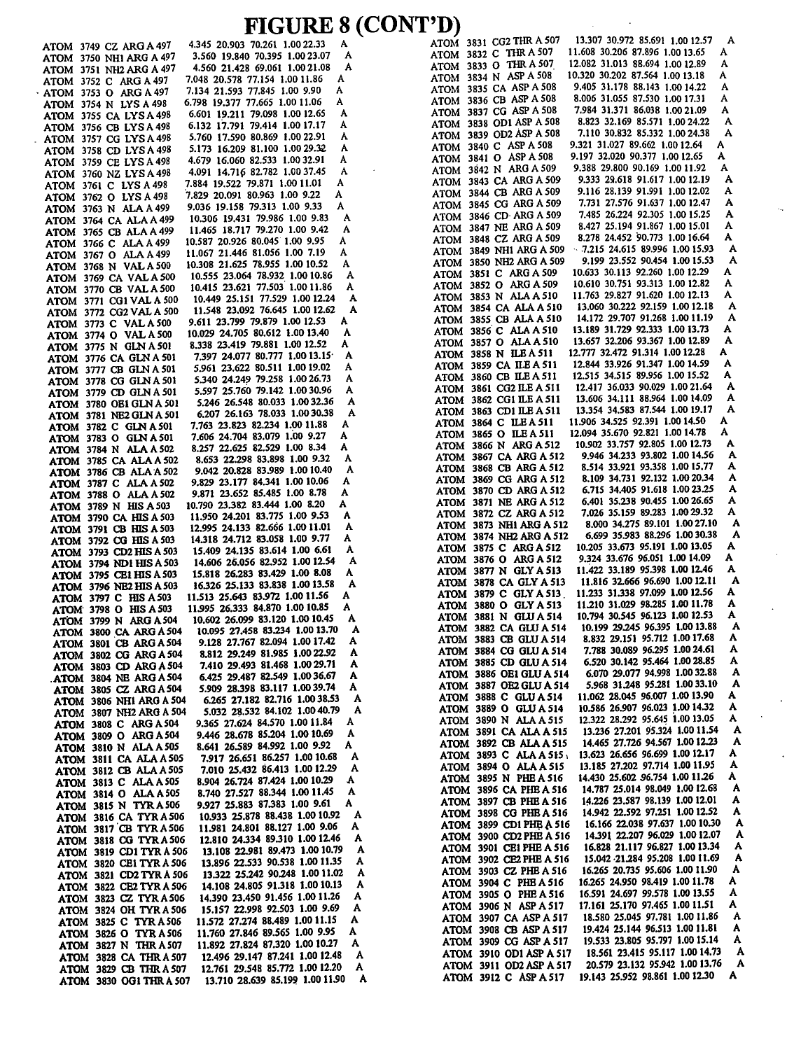

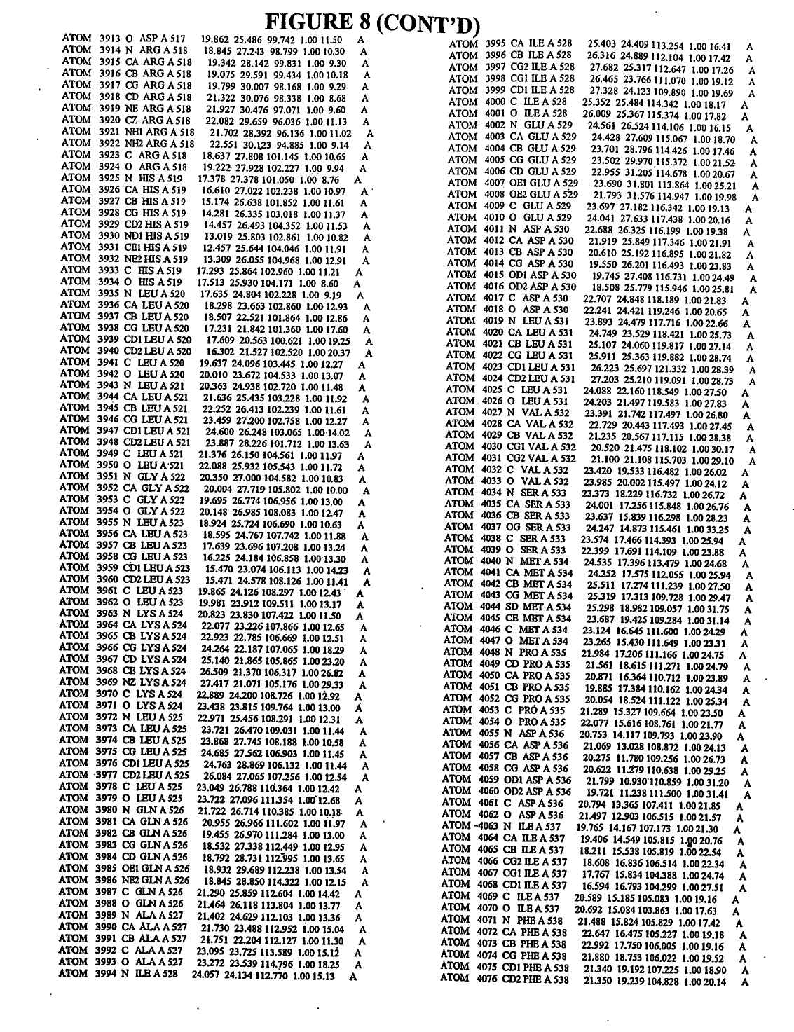

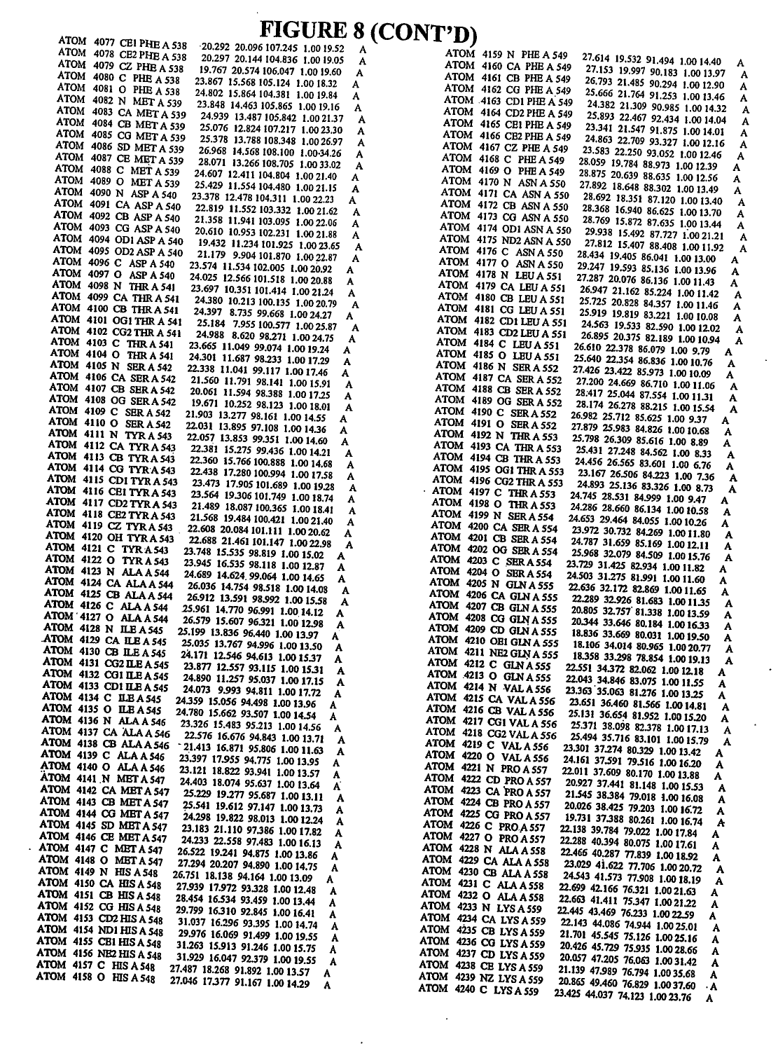

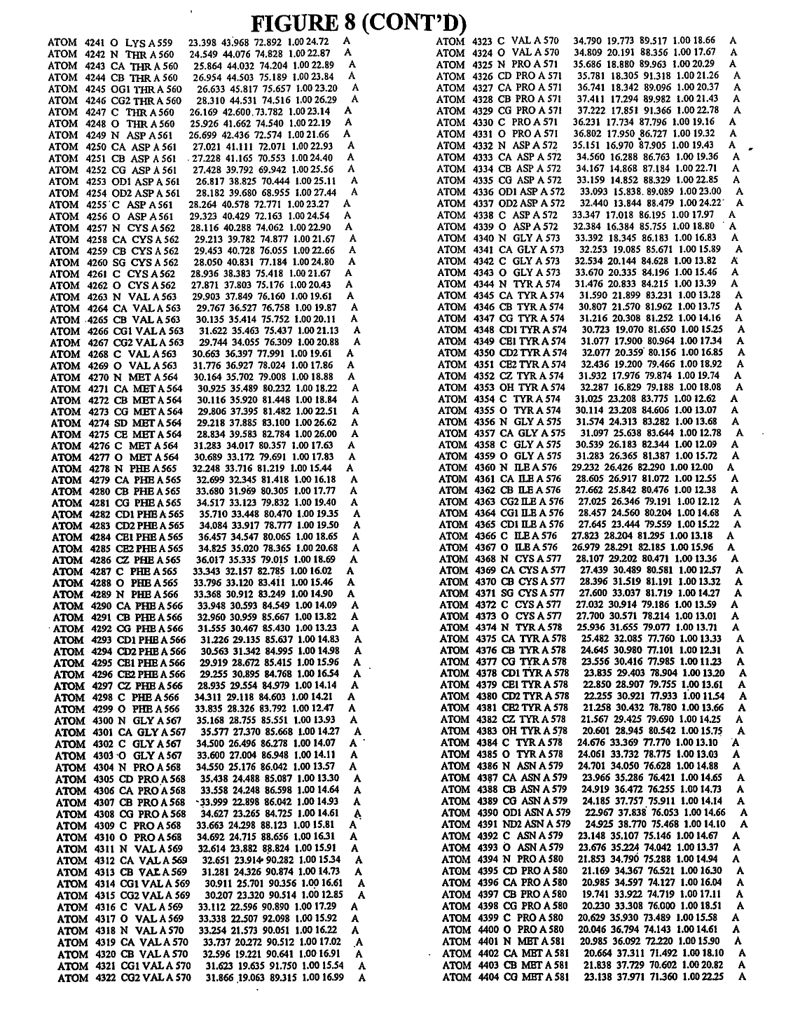

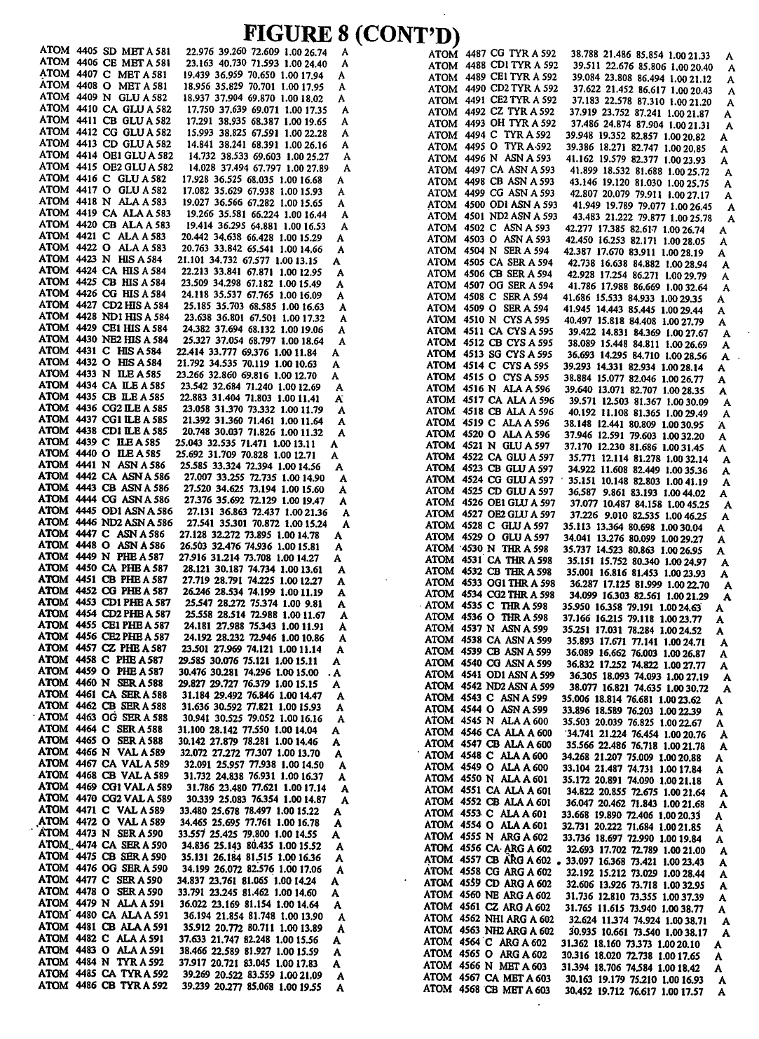

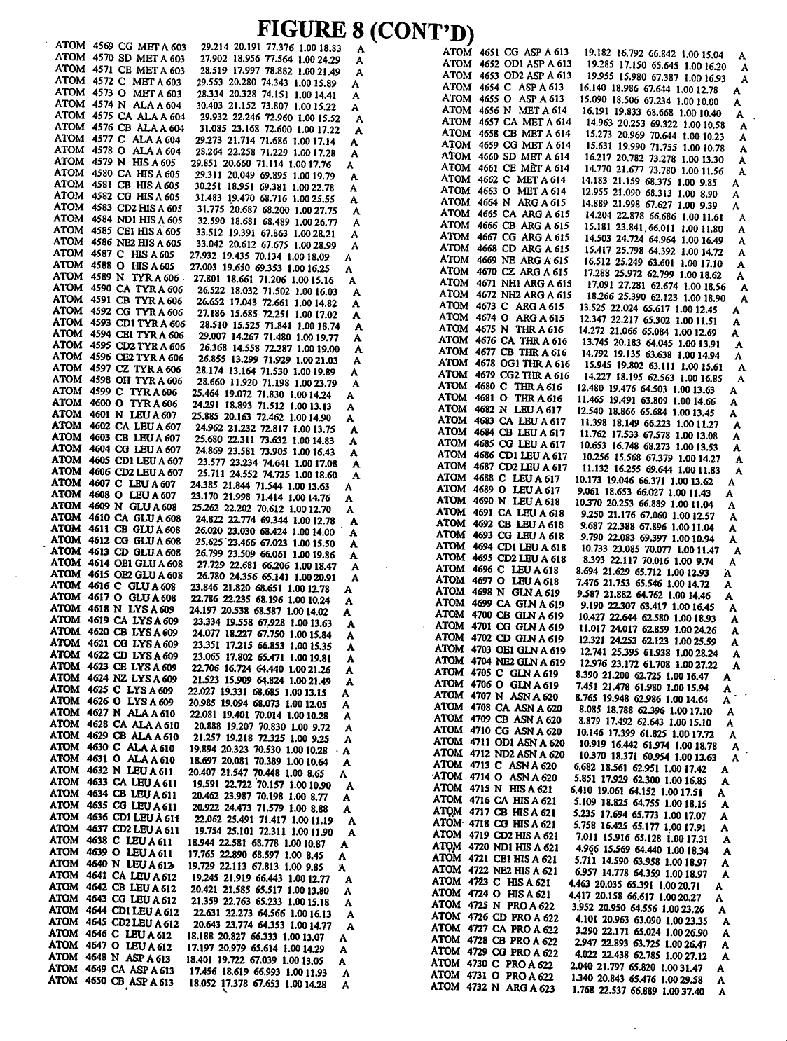

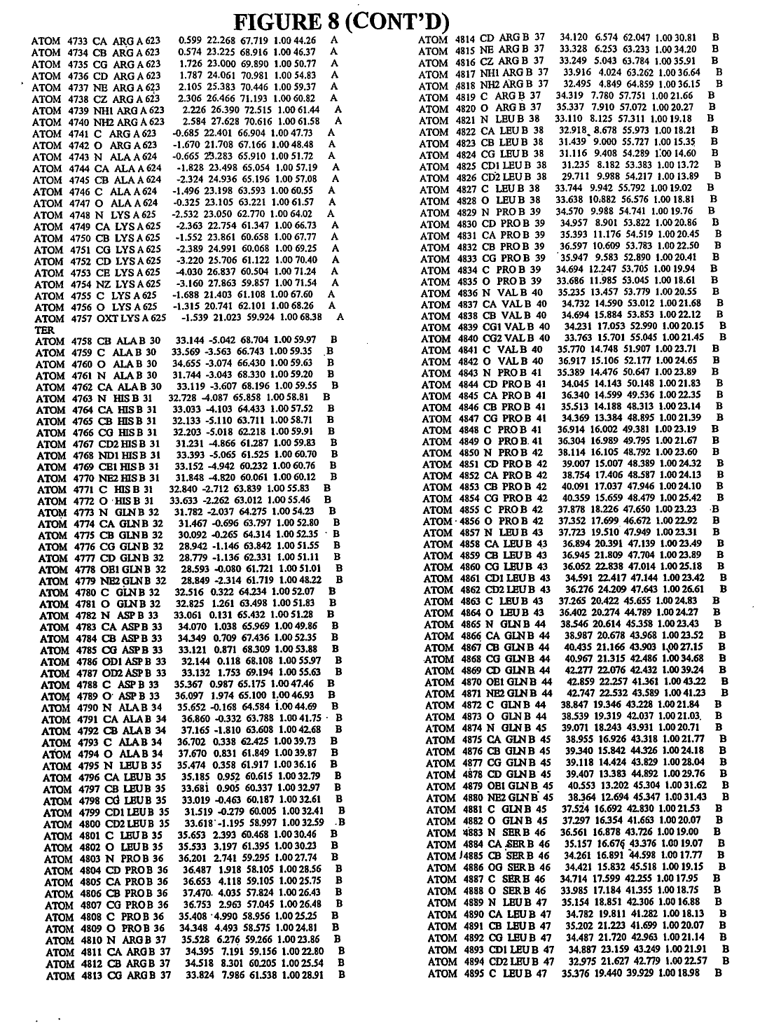

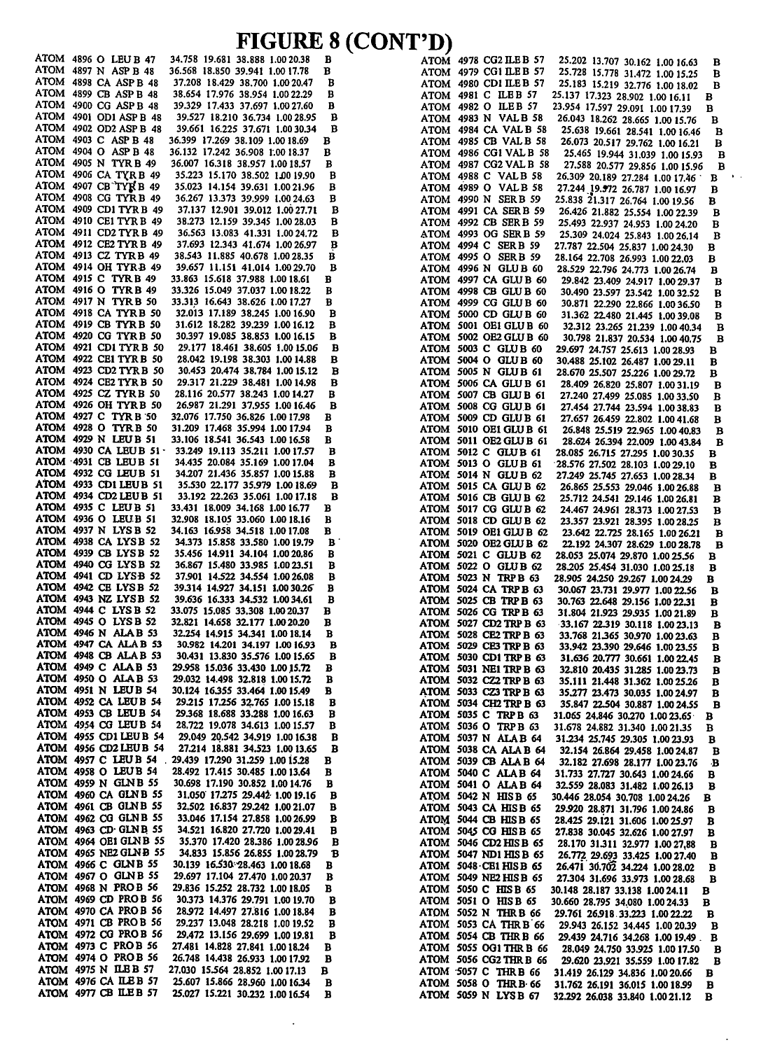

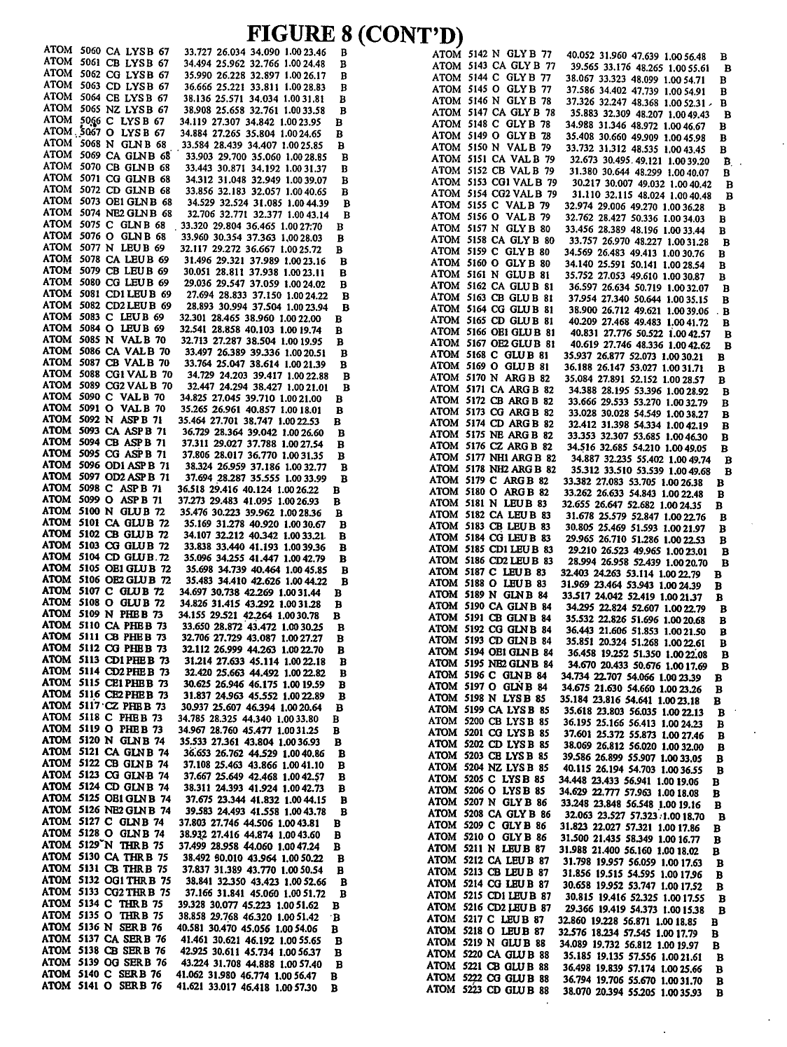

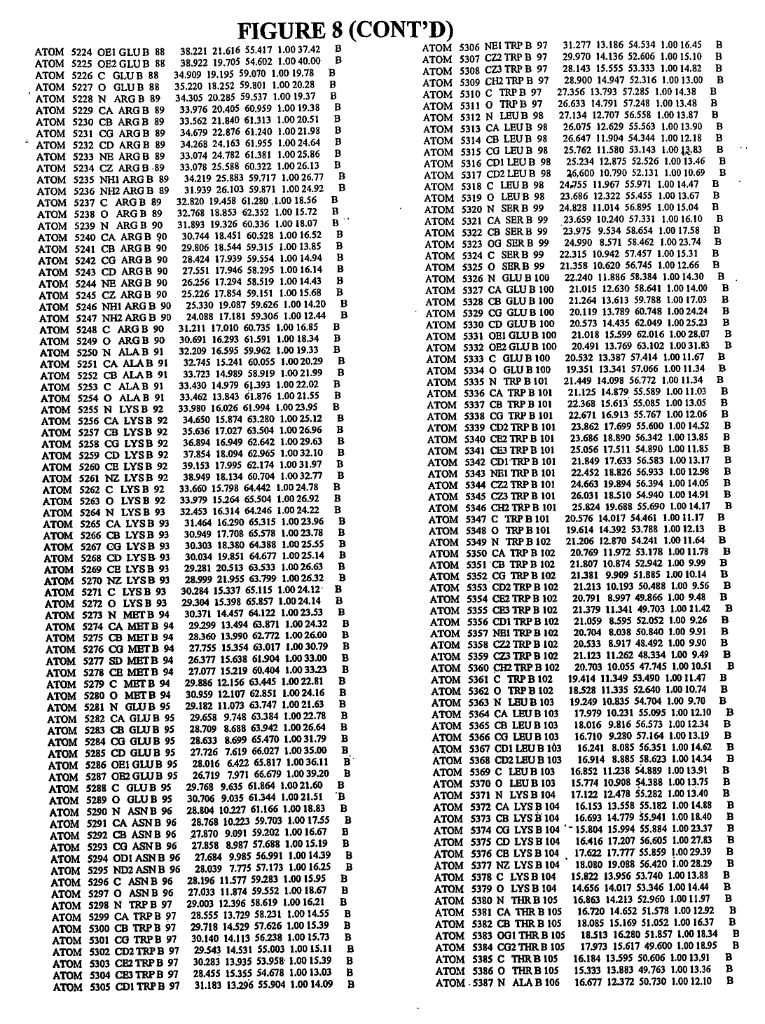

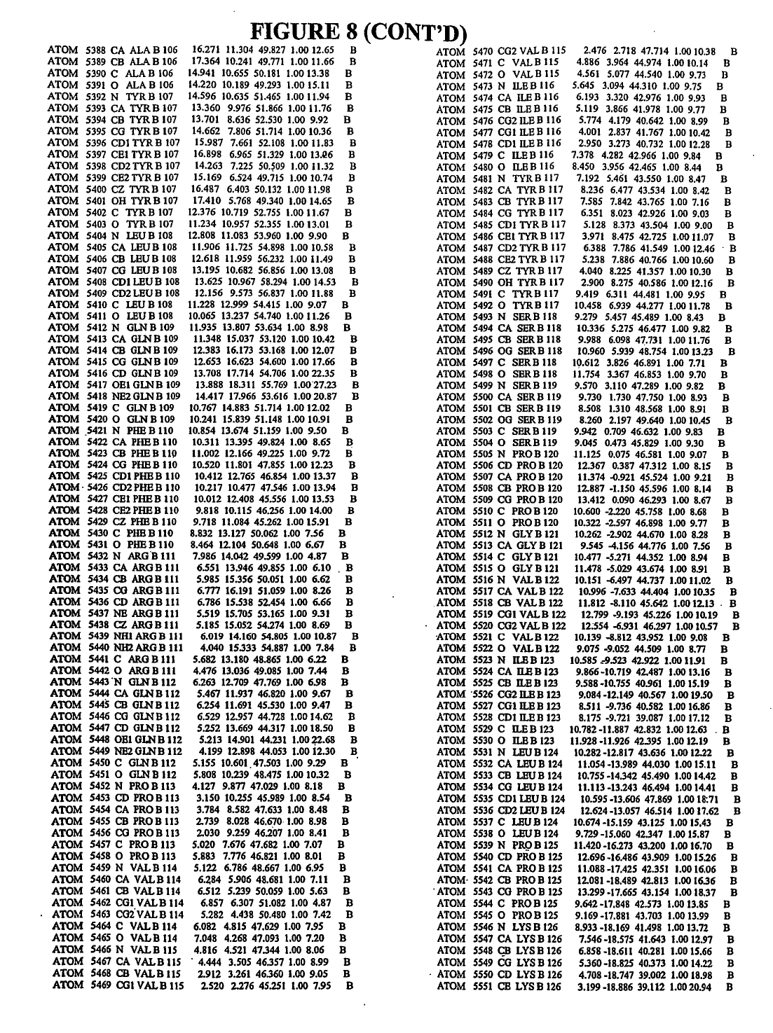

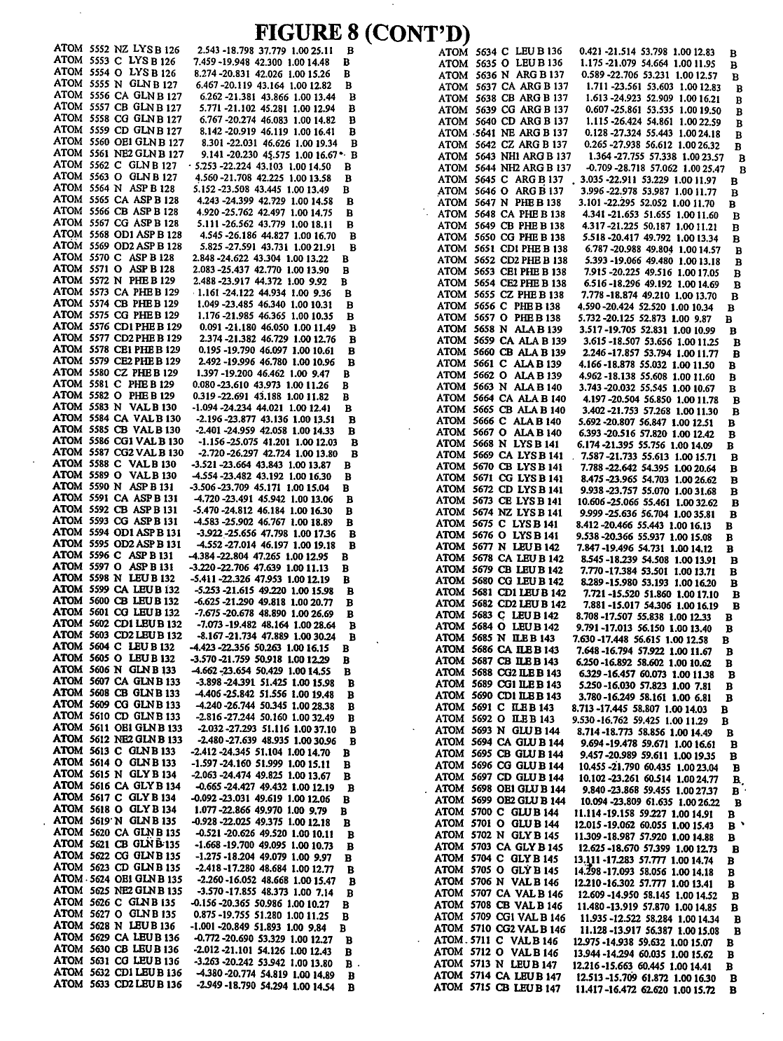

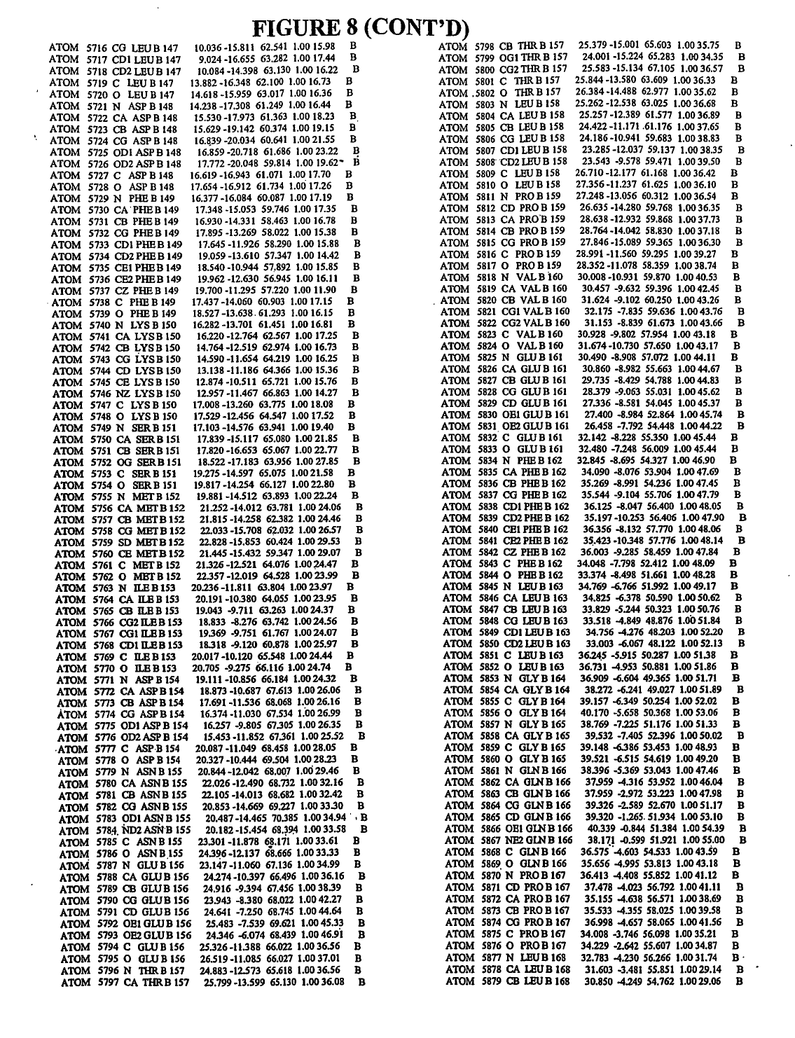

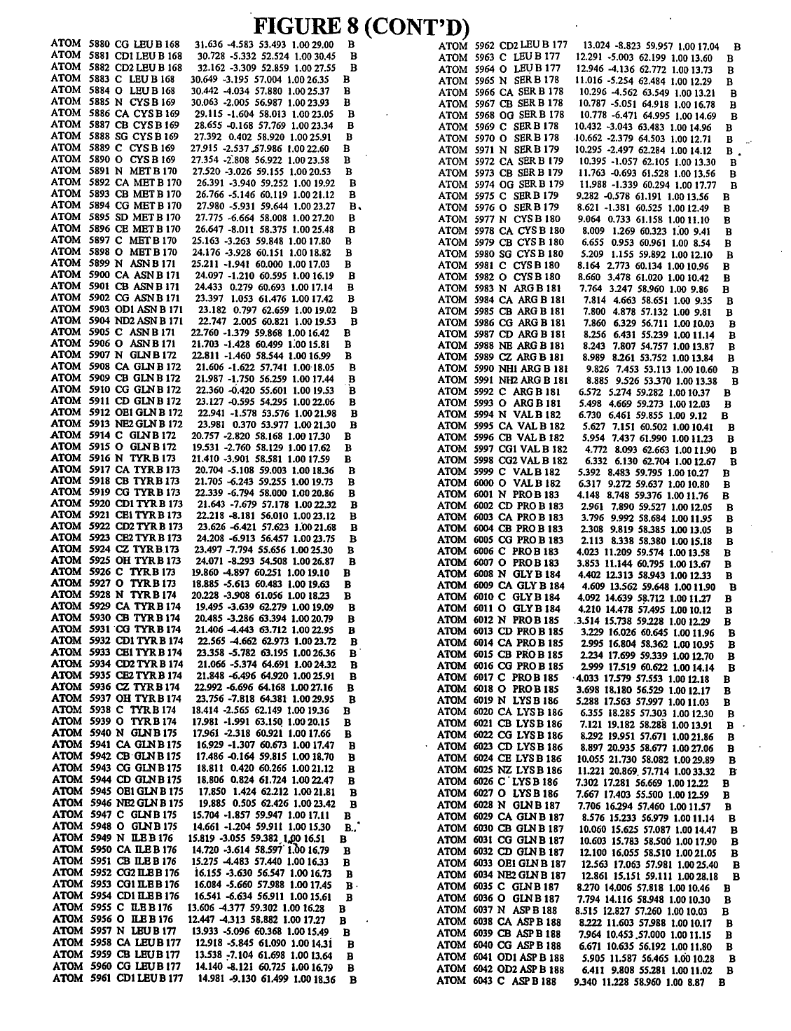

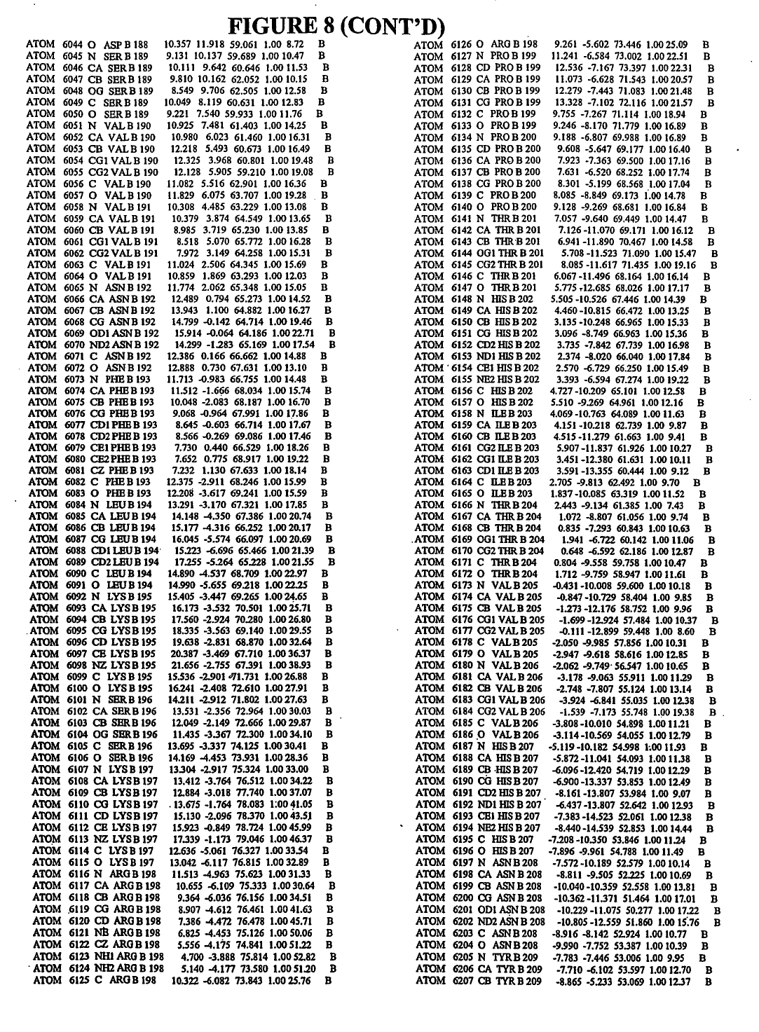

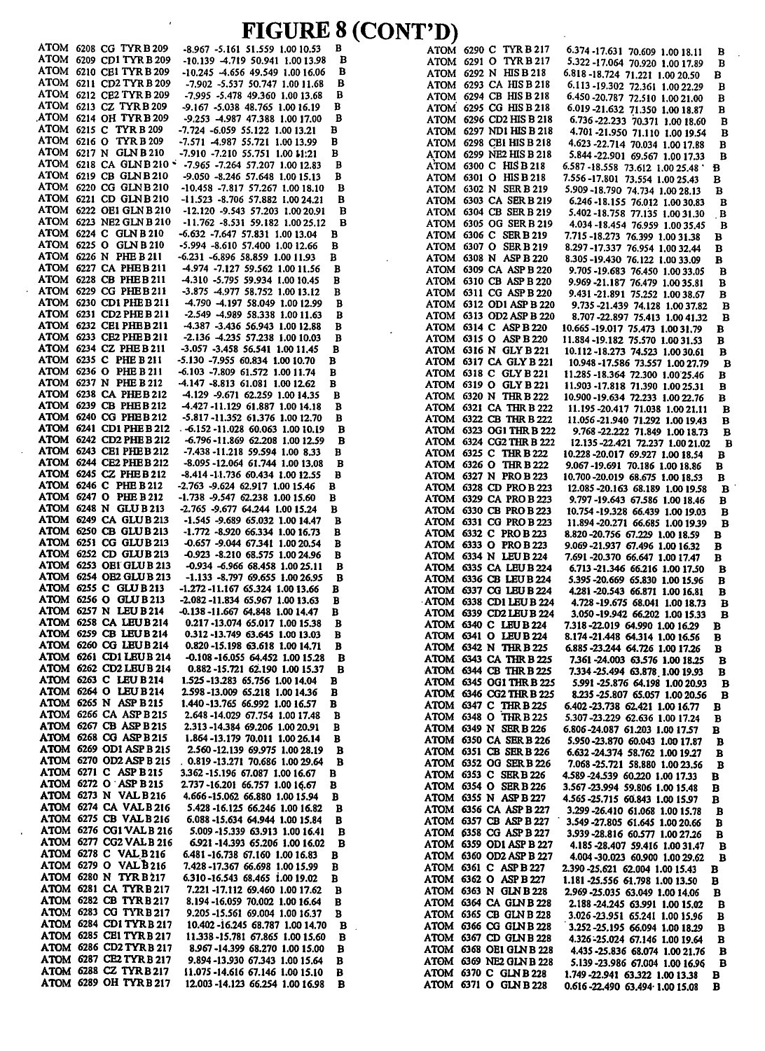

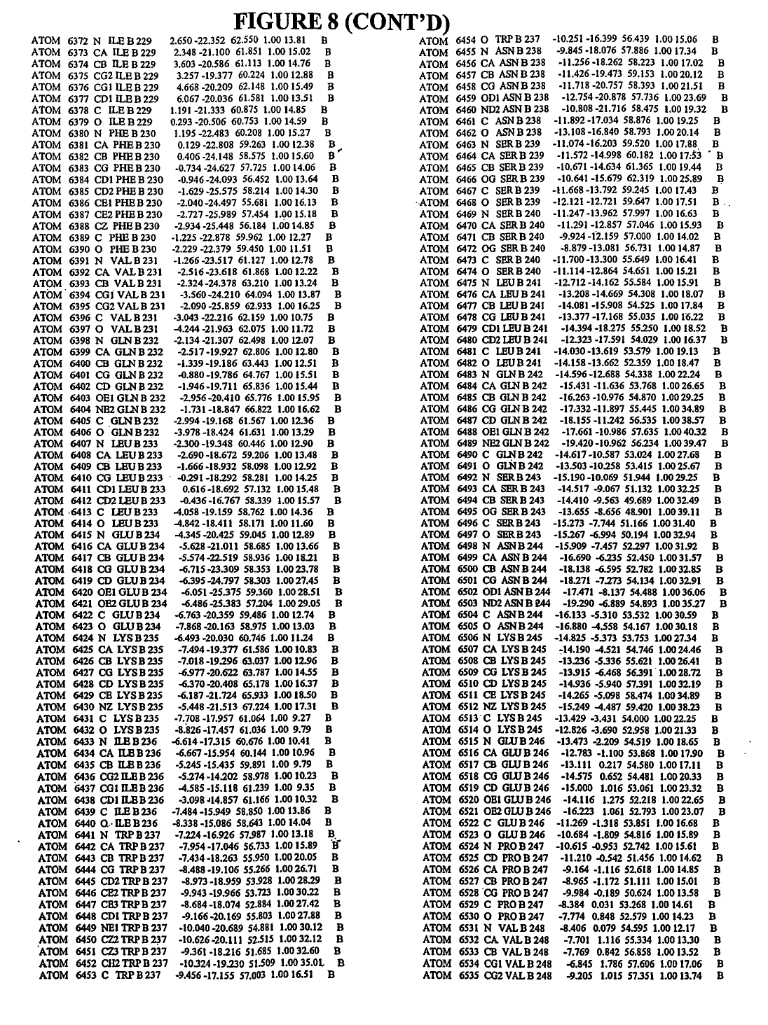

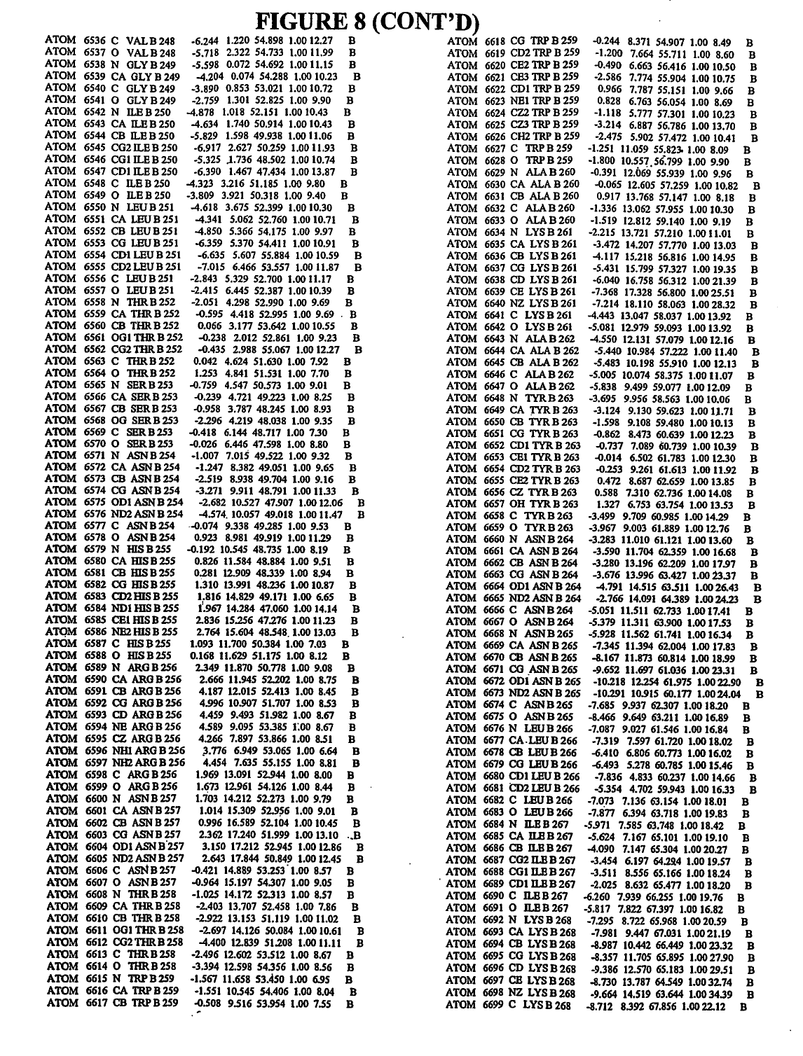

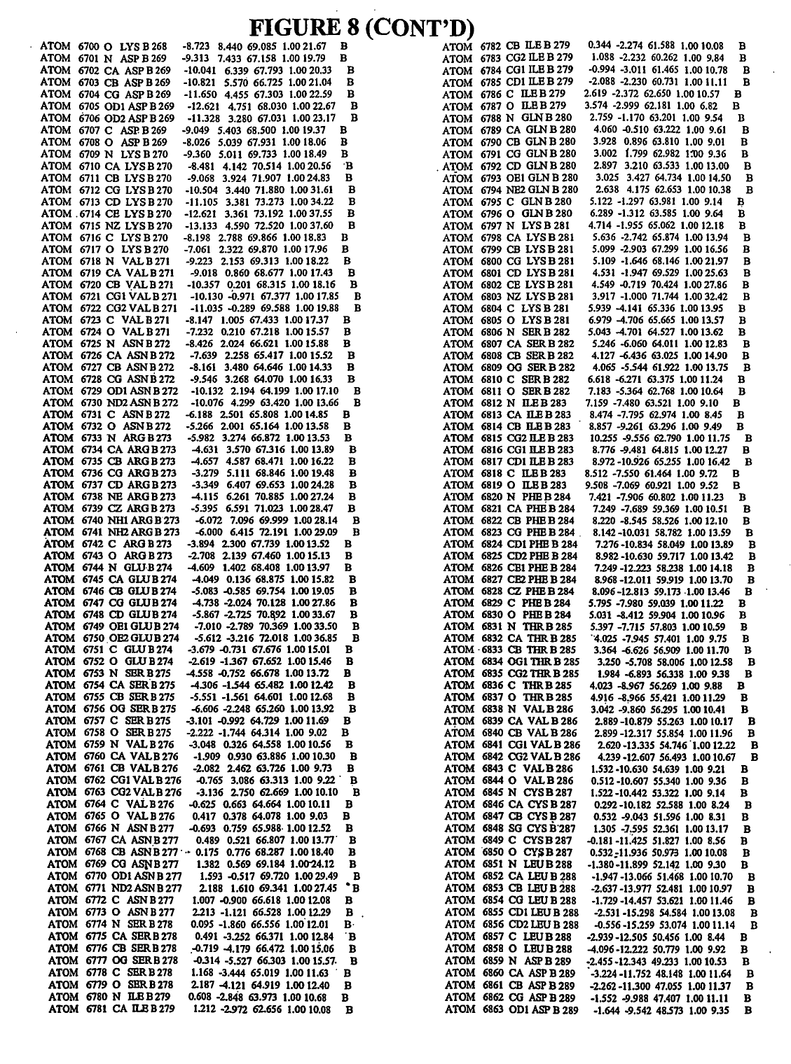

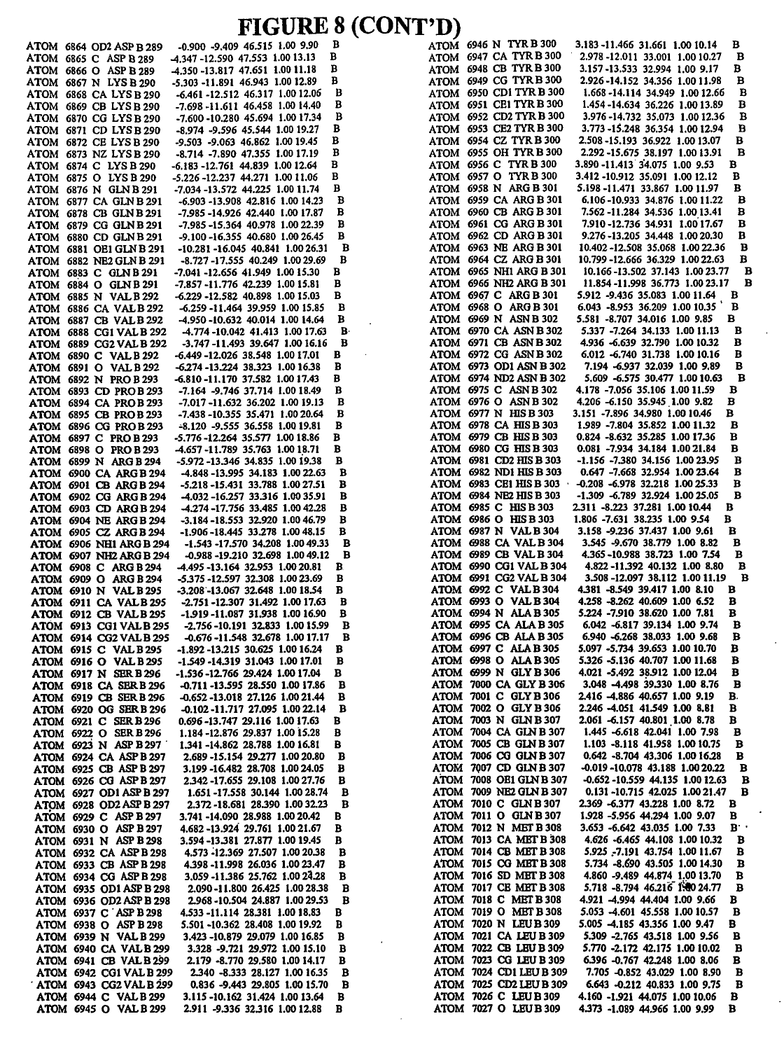

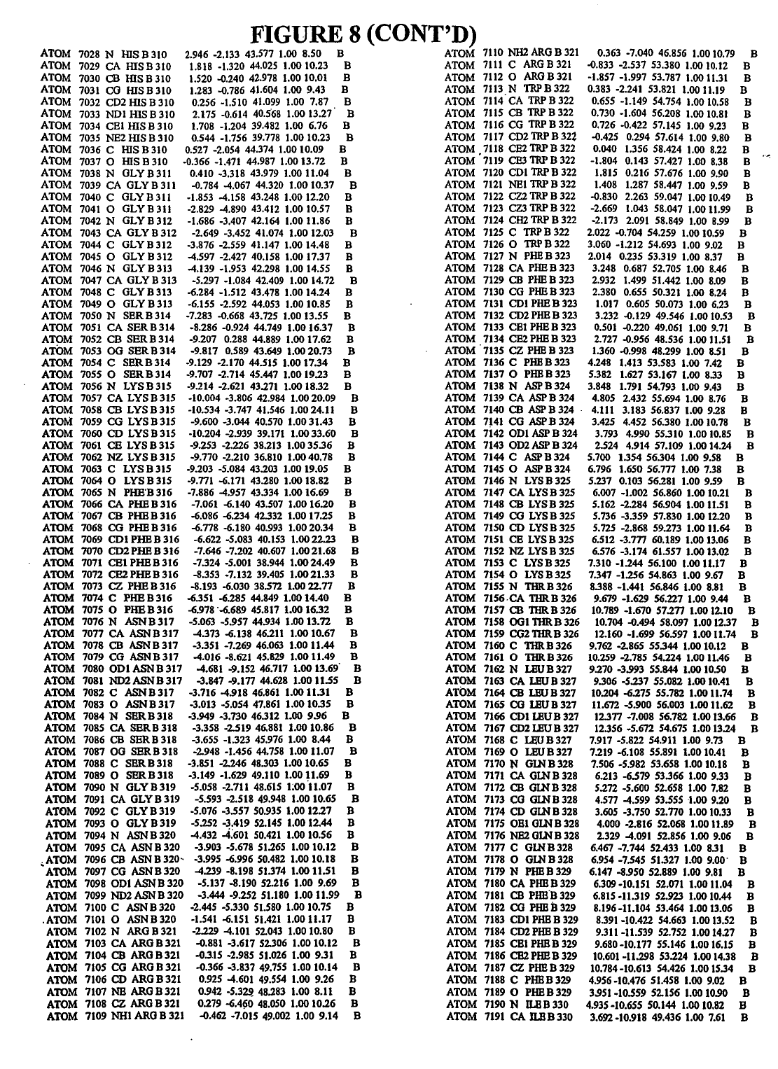

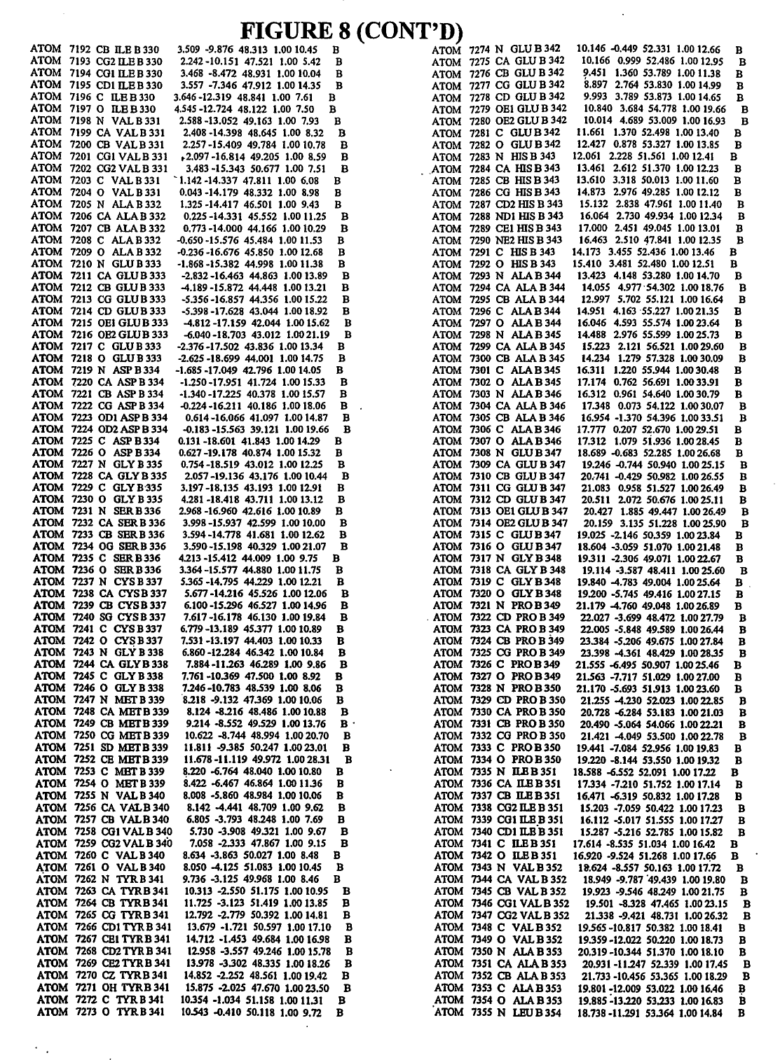

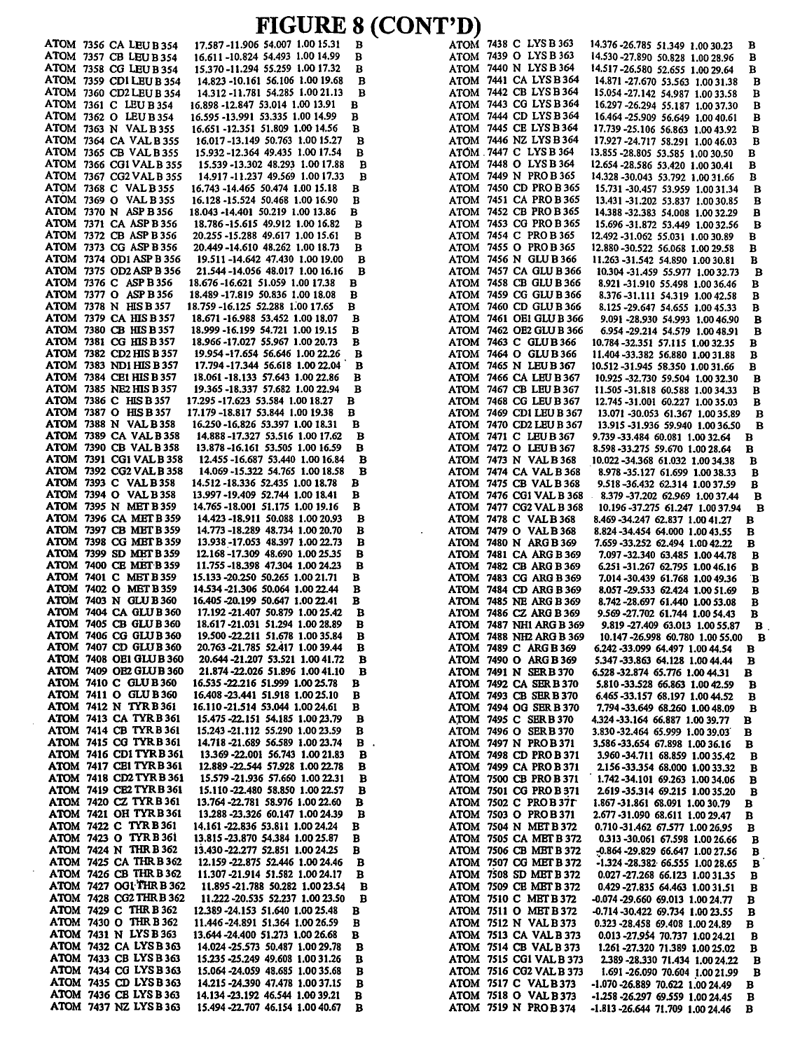

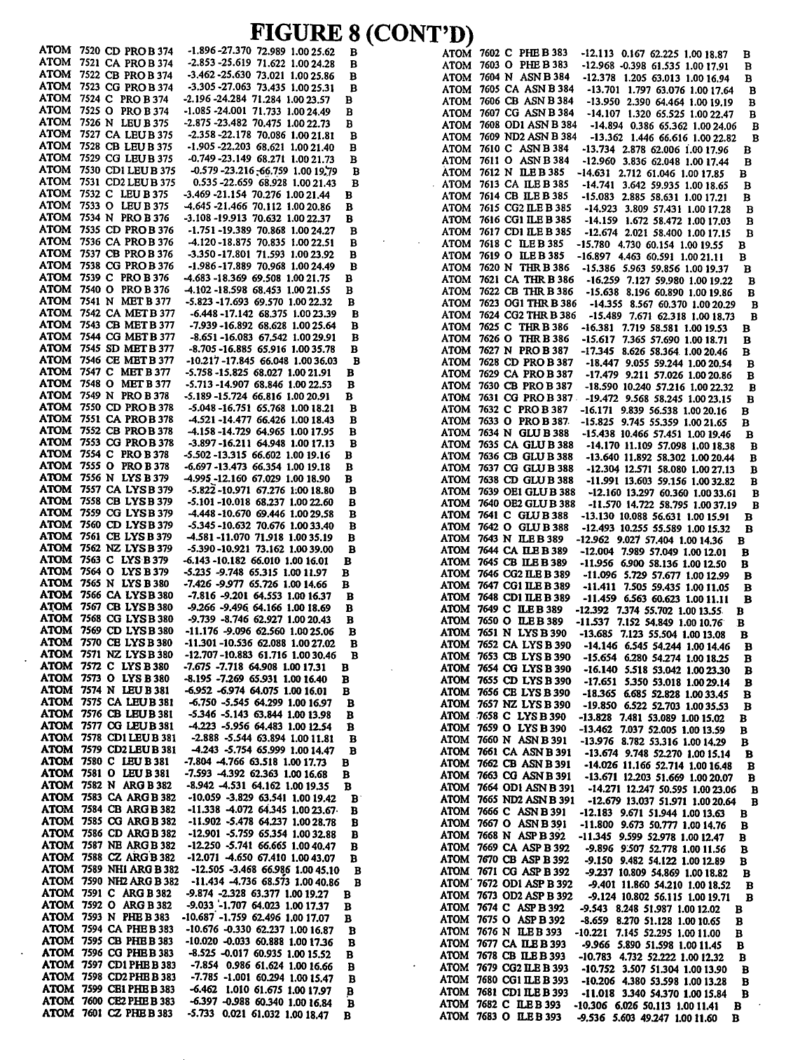

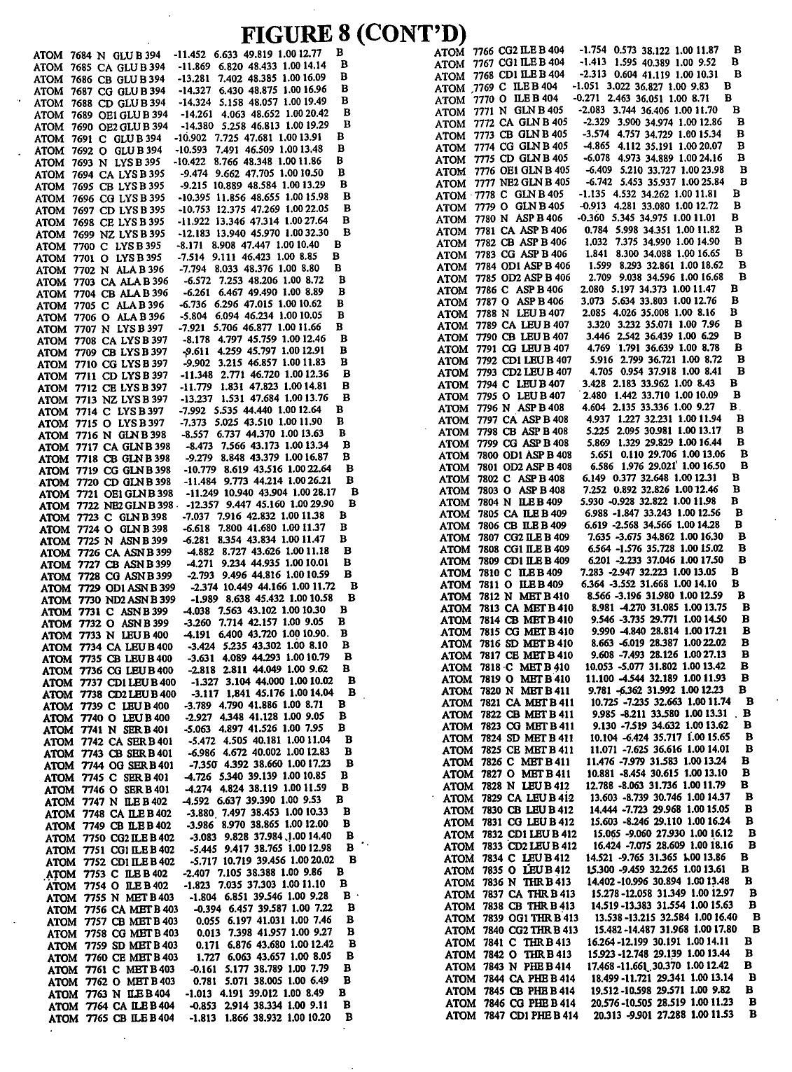

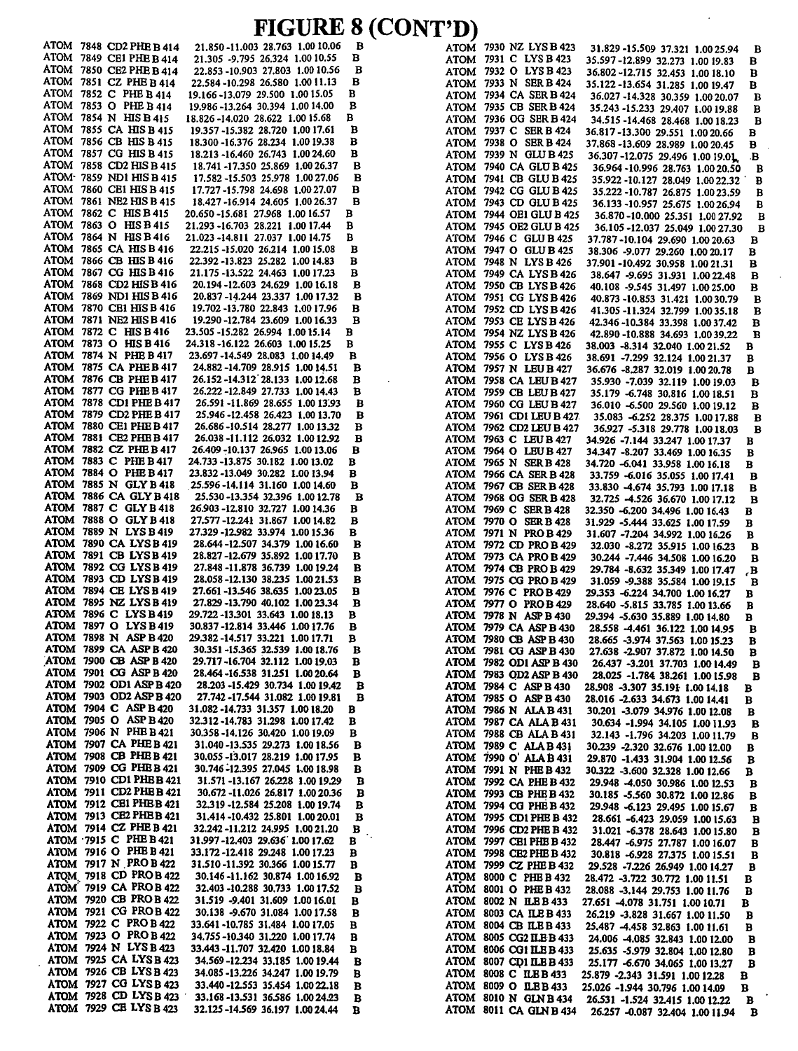

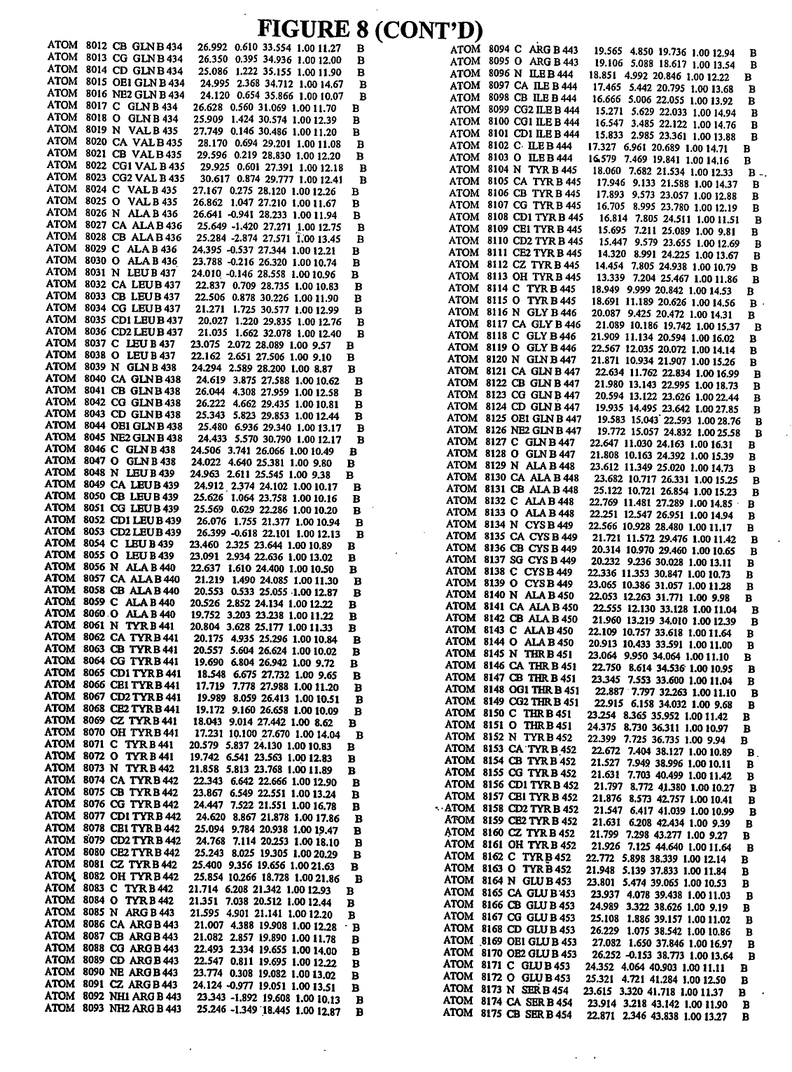

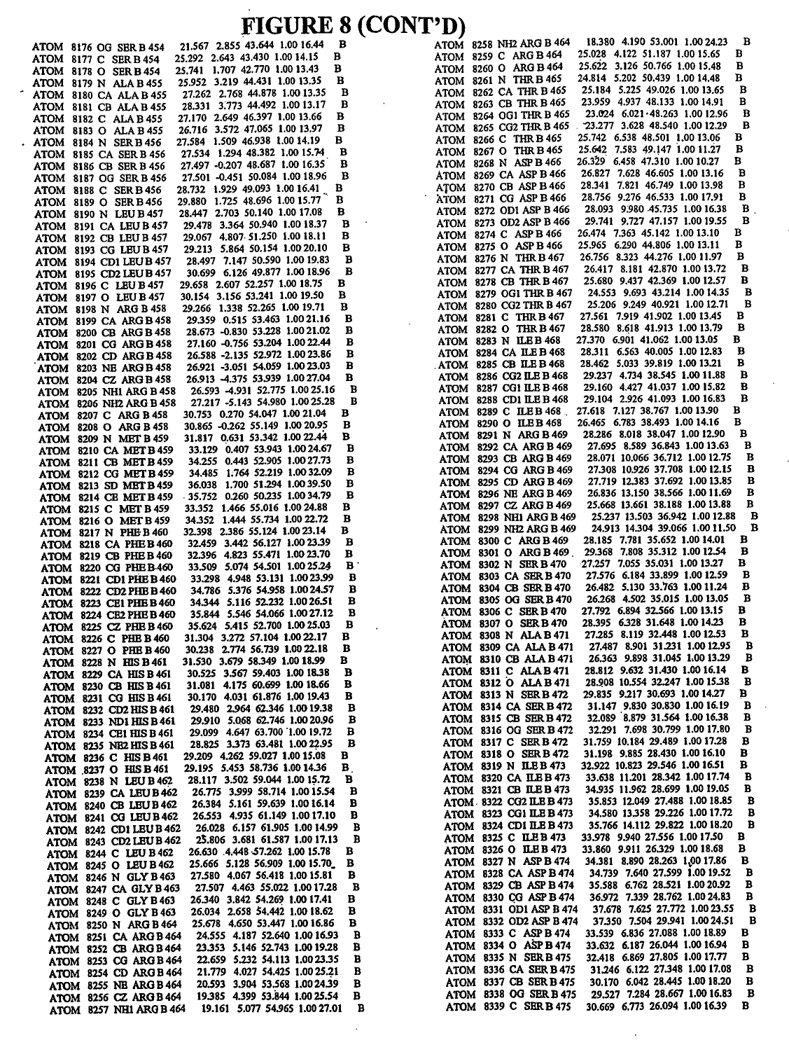

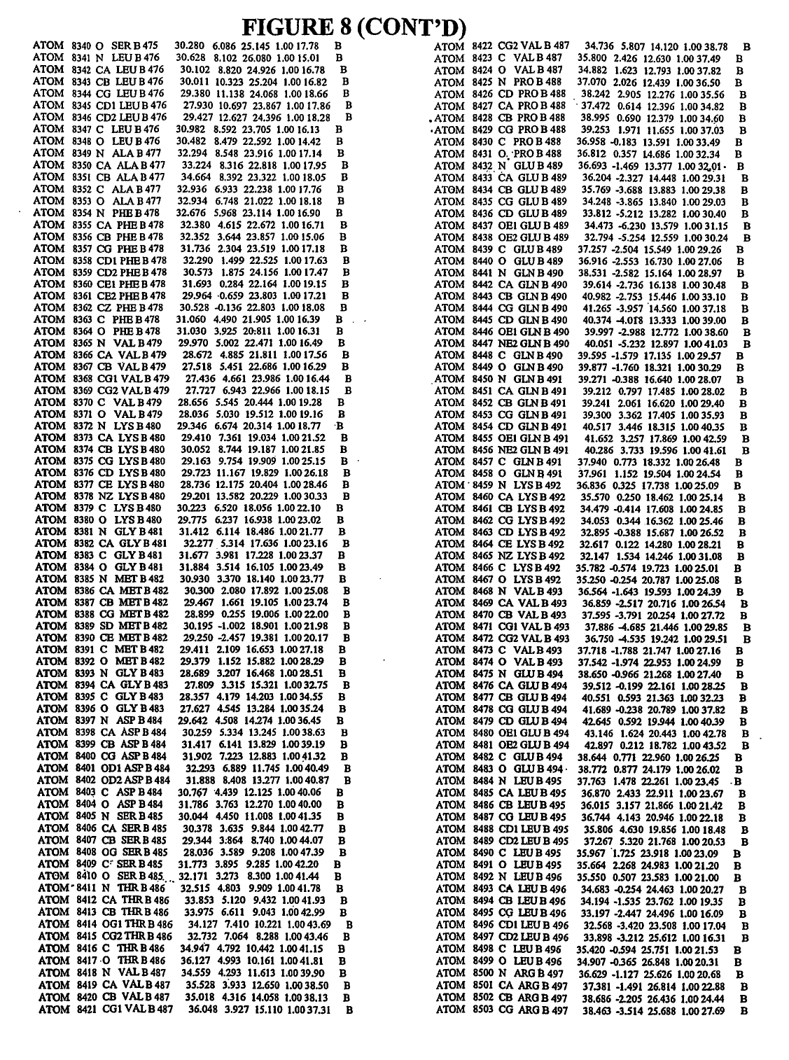

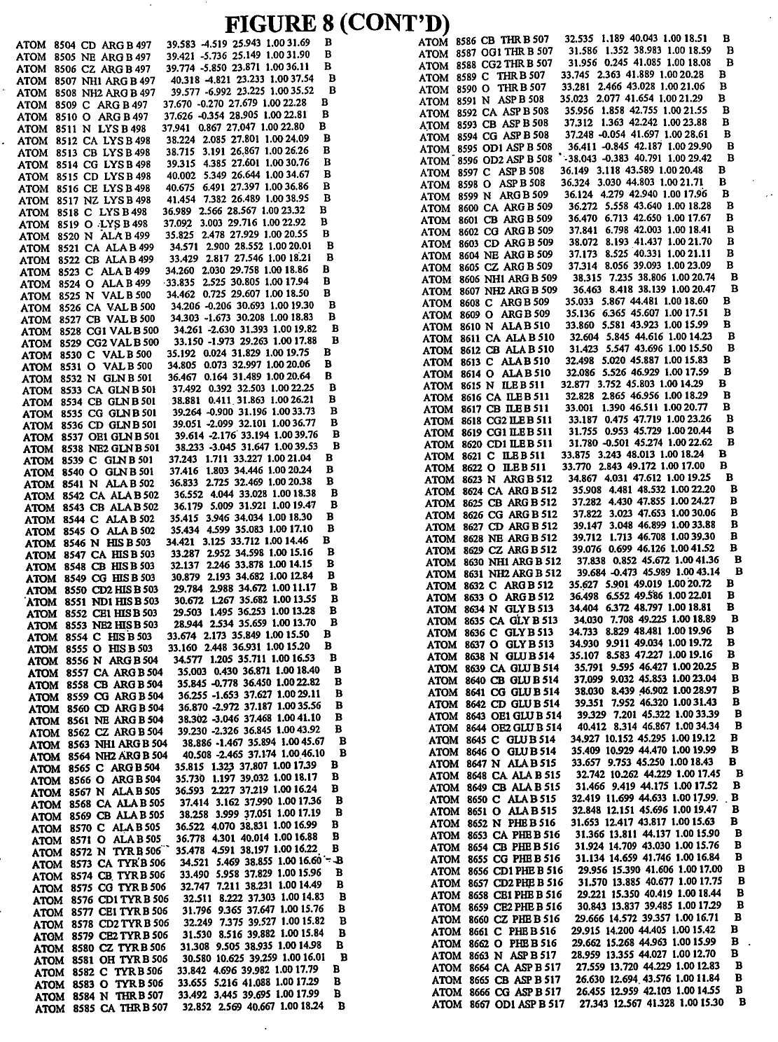

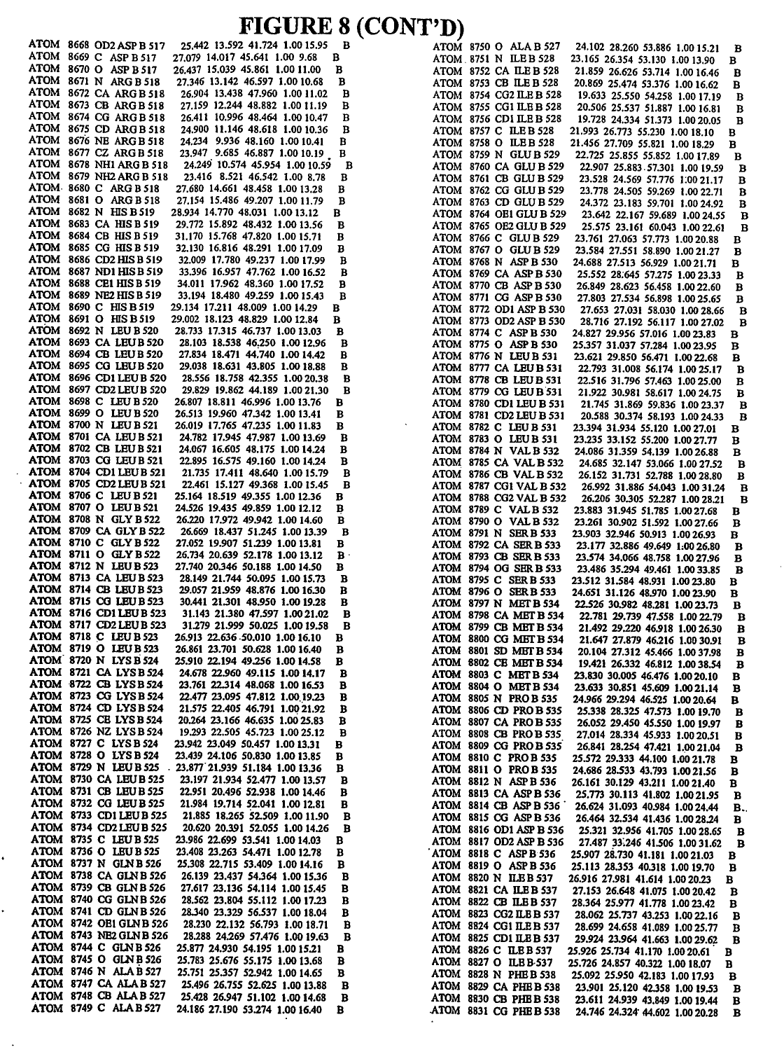

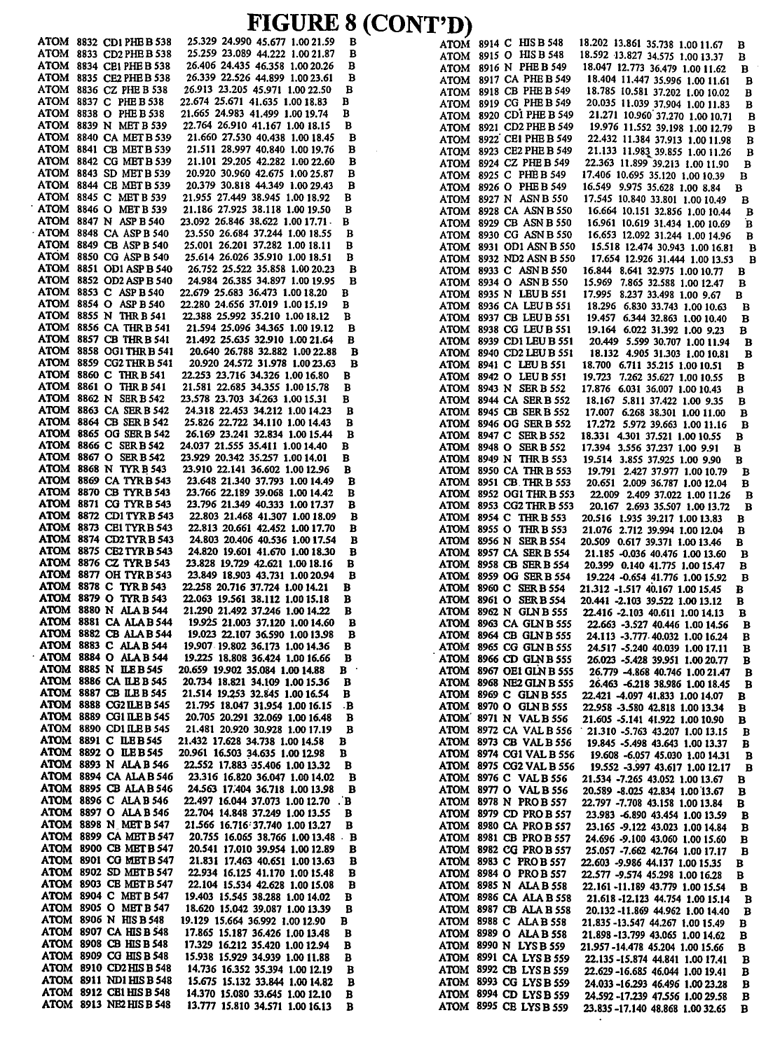

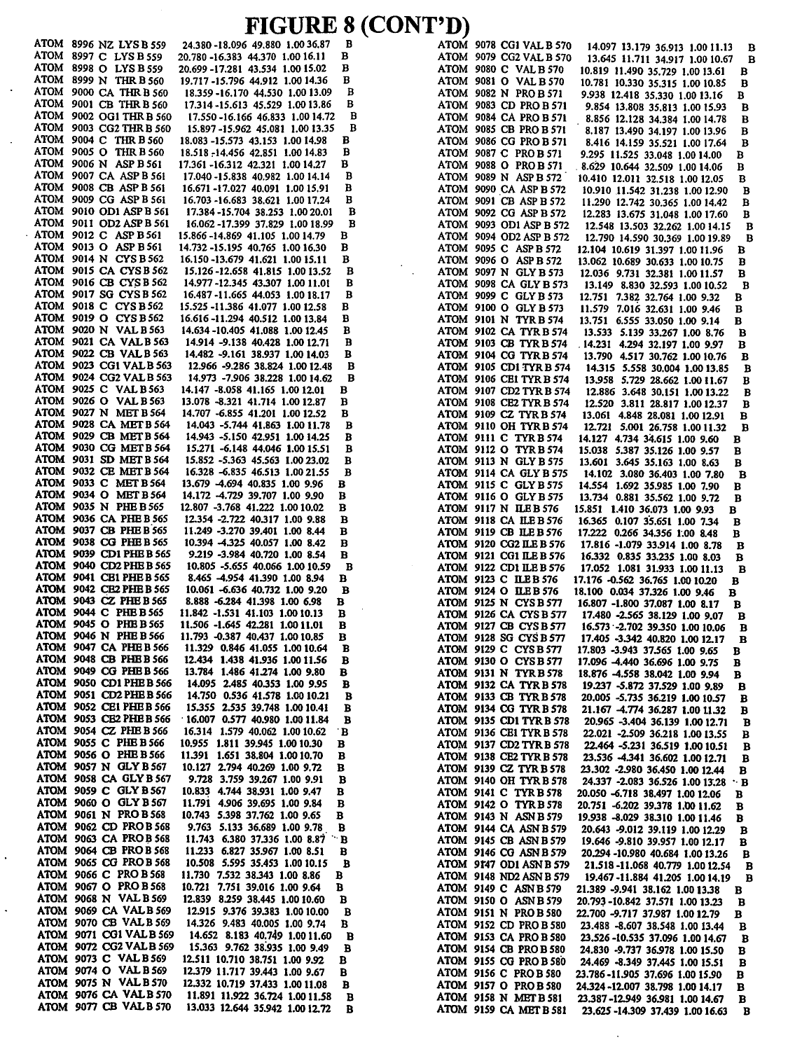

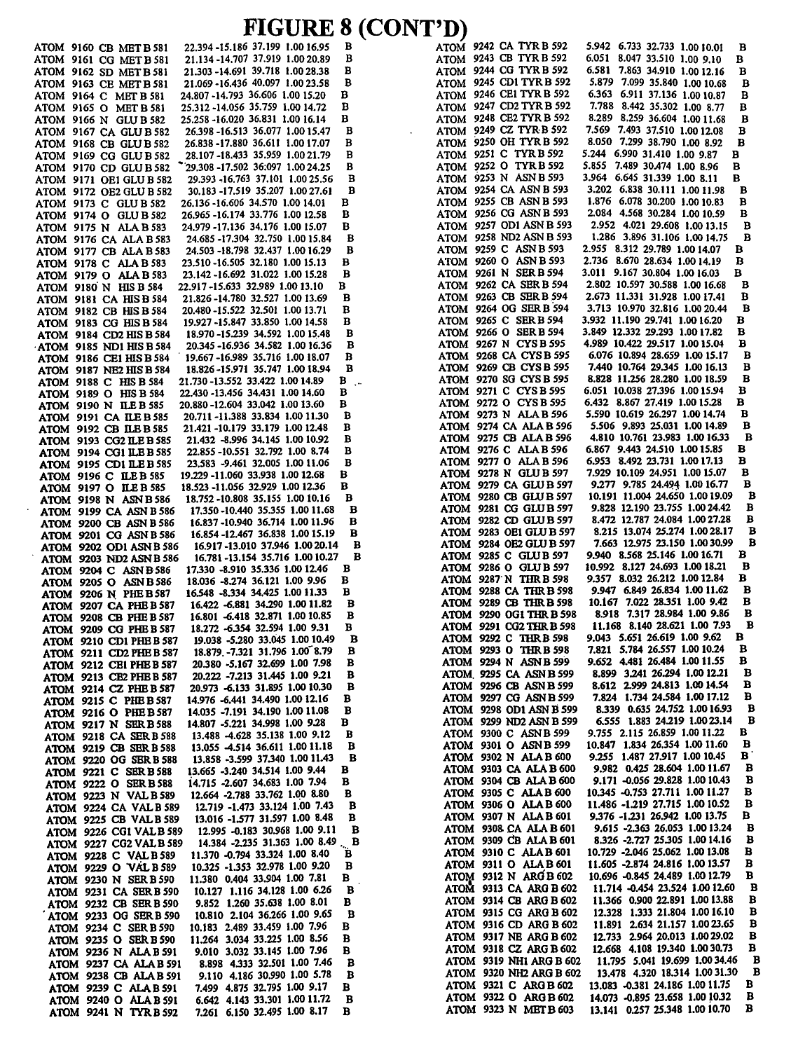

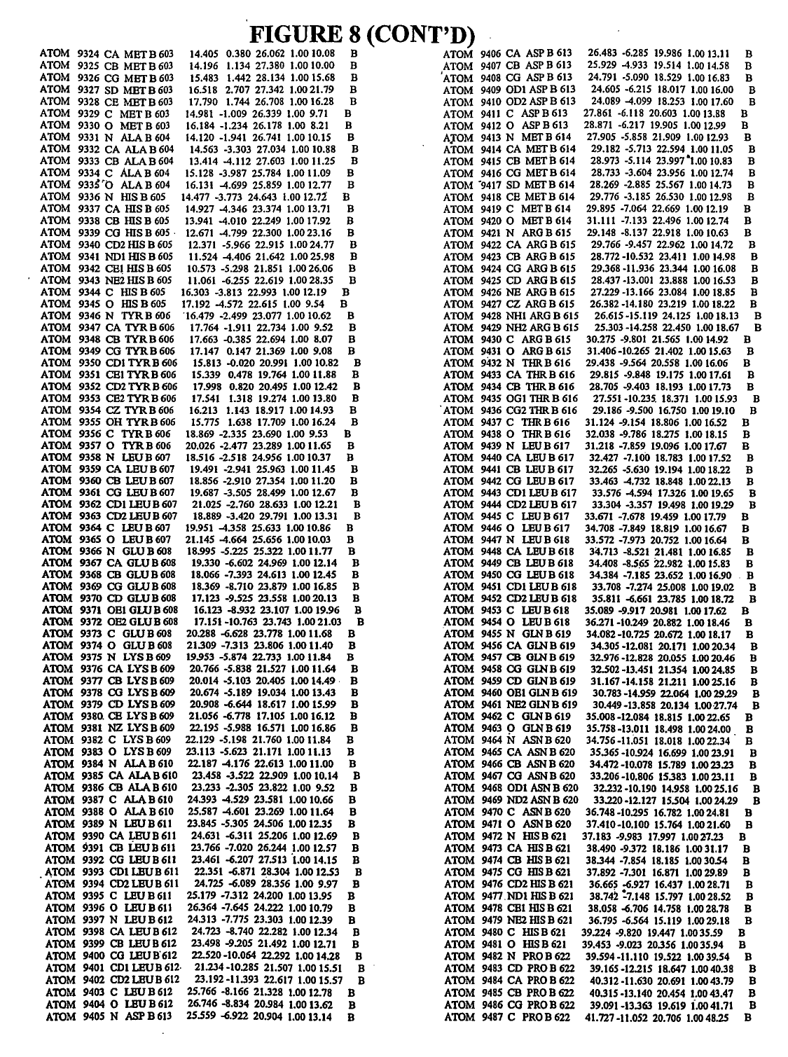

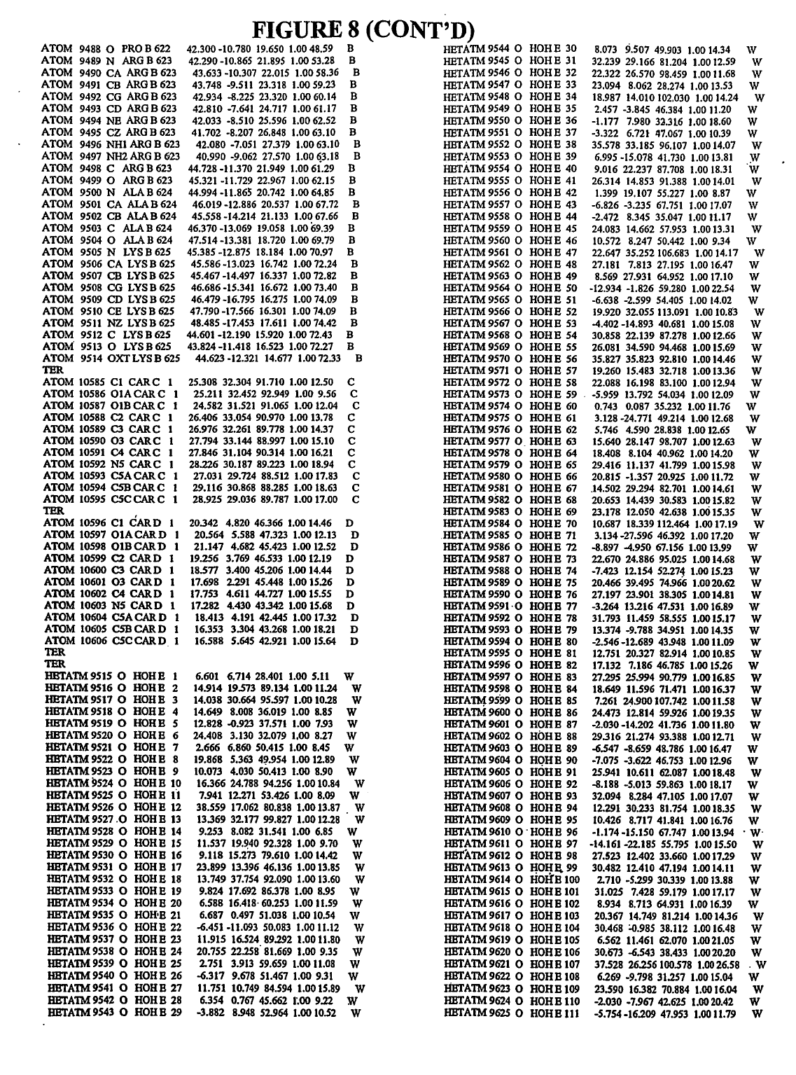

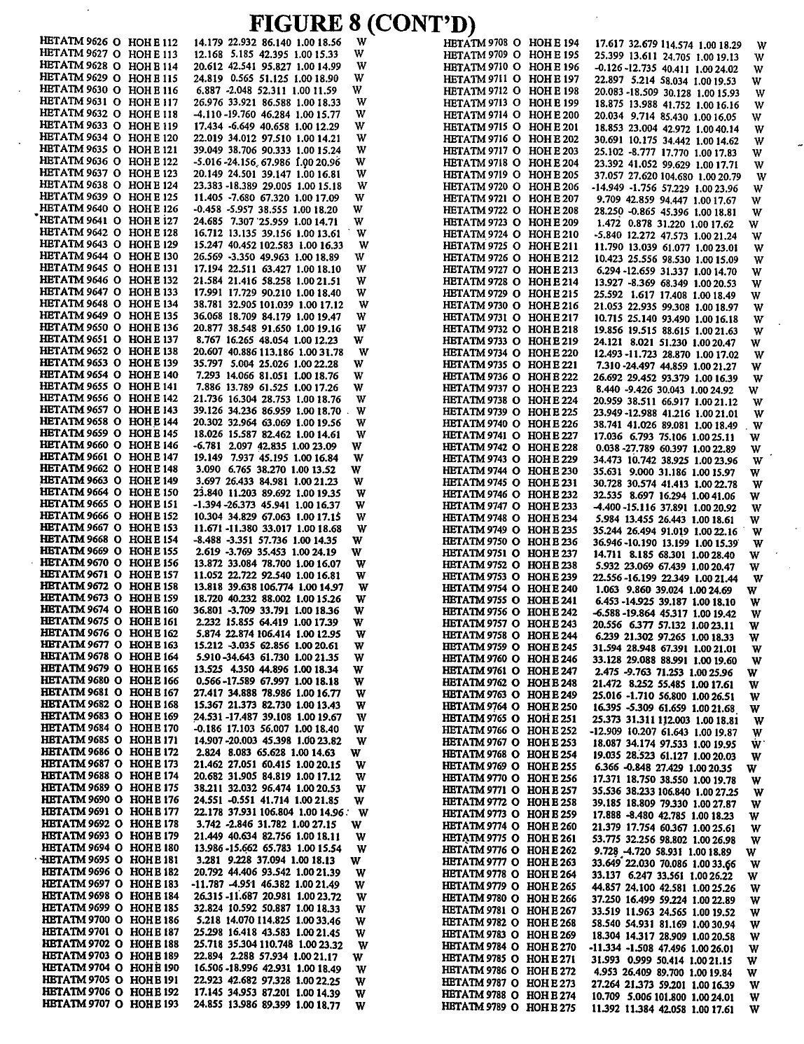

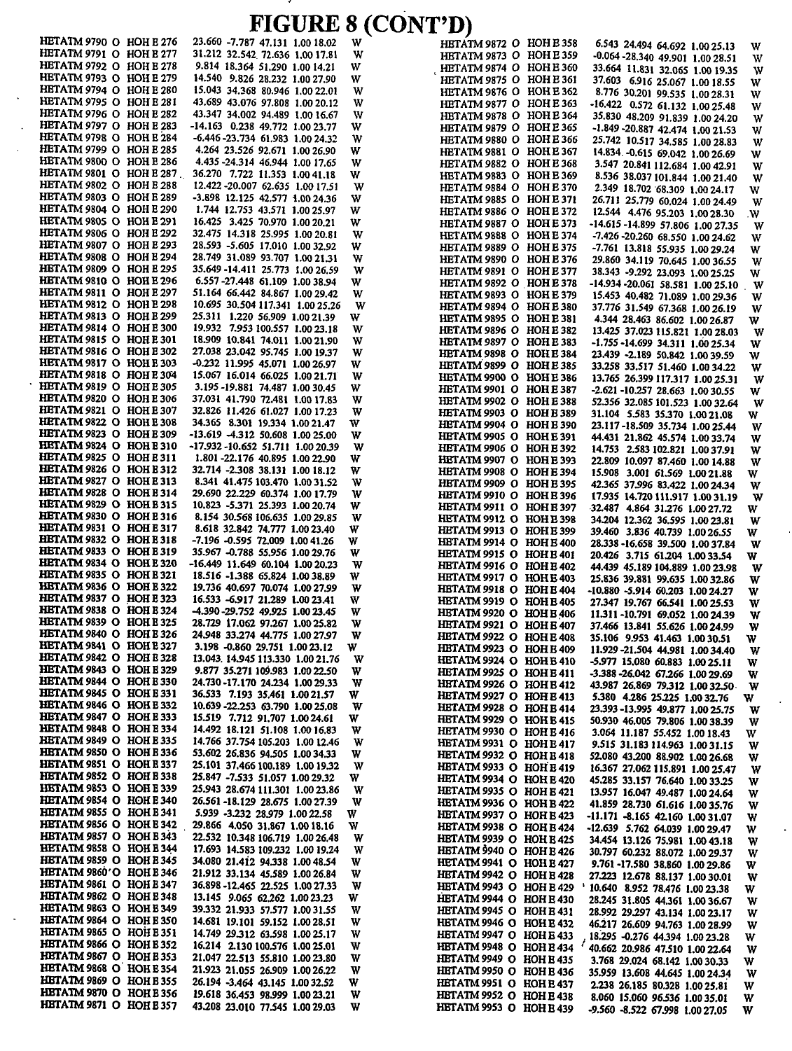

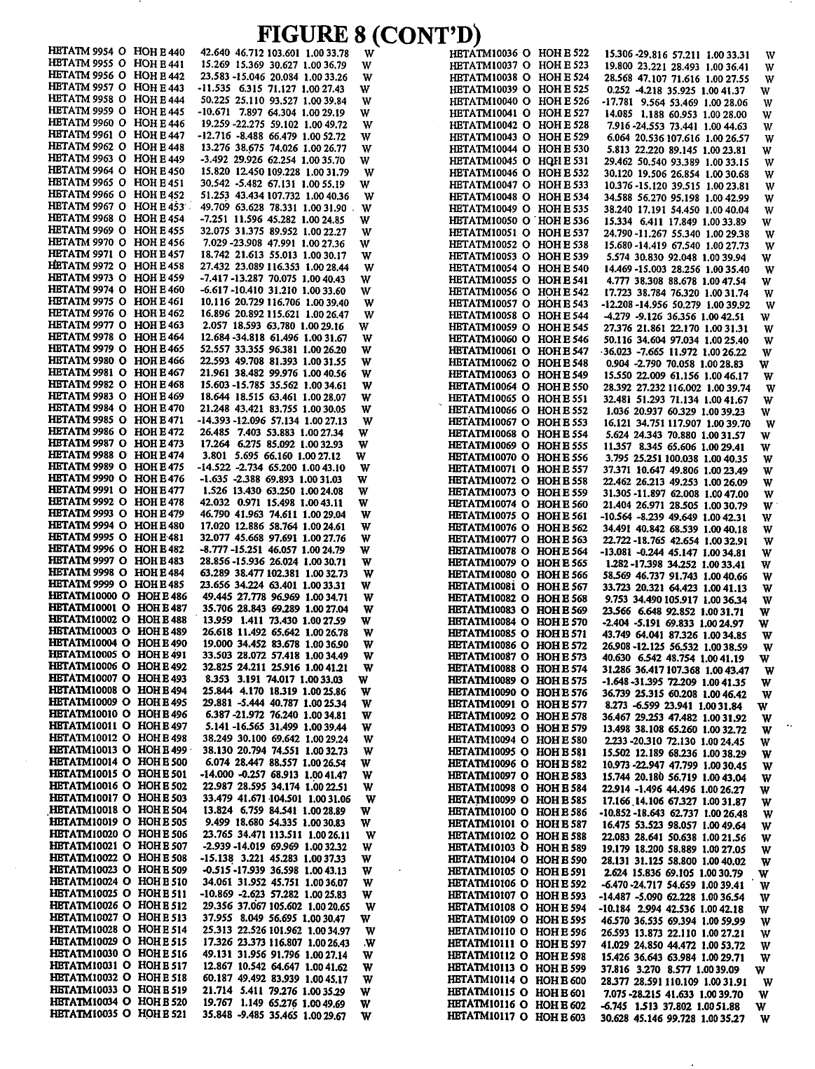

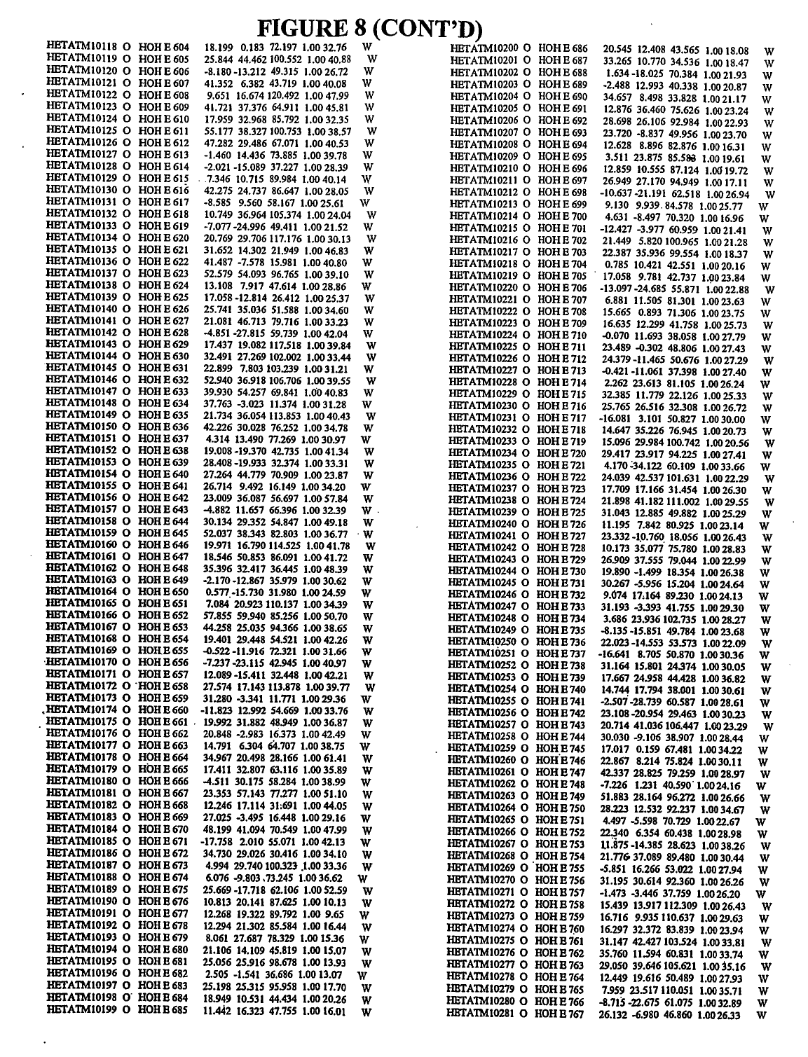

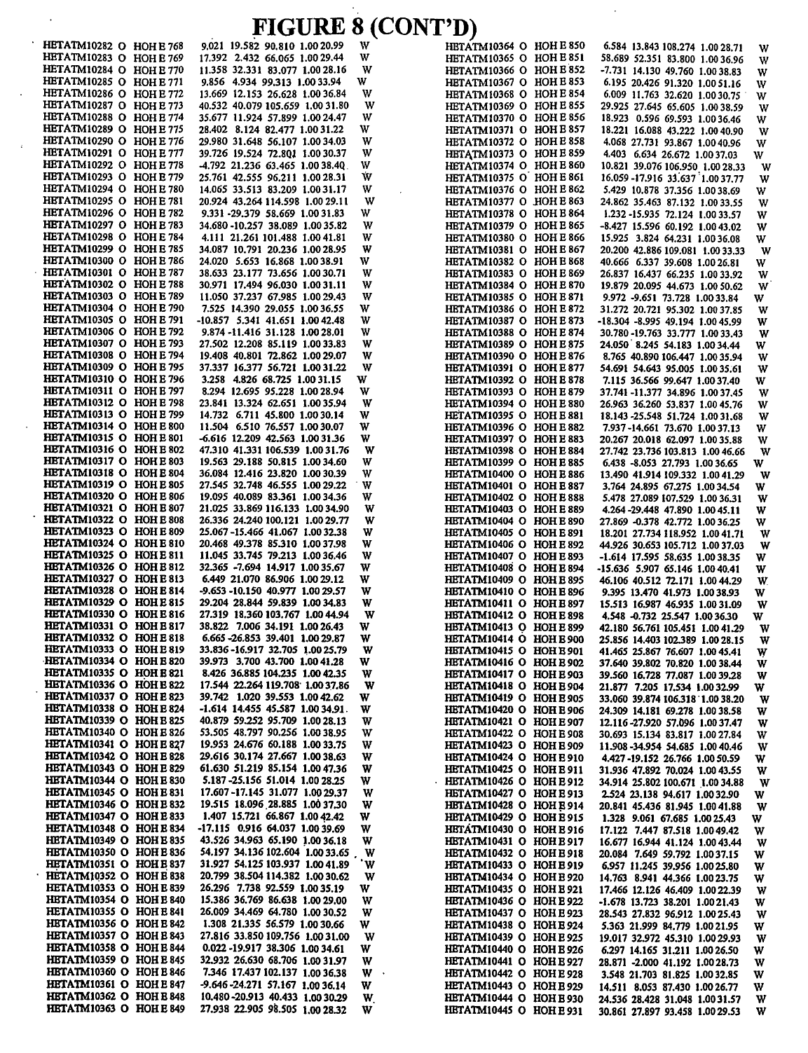

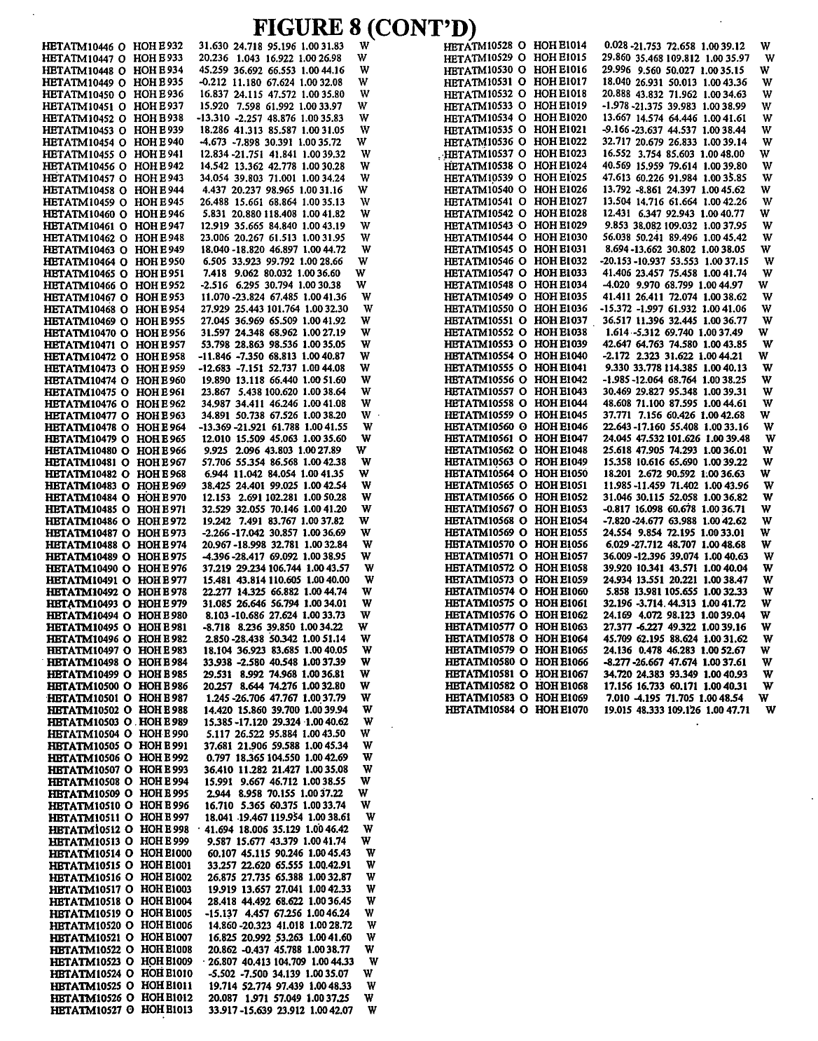

The term “substrate binding site” refers to the region of a carnitine acyltransferase that retains substrate in a position suitable for acyl transfer to occur. The configuration of the substrate binding site is likely to be different in the presence and absence of bound substrate, and both configurations are optimally considered in the design and/or selection of enzyme modulators. Specifically, for mCRAT, the carnitine binding site is schematically depicted in FIG. 3 and the CoA binding site is schematically depicted in FIG. 4. In one preferred set of non-limiting embodiments of the invention, the carnitine binding site of CRAT is comprised in a protein structure comprising β-sheet (strands β11-β14) in the C domain, and residues in α5-β1 and β8-α12 in the N domain, as depicted for mCRAT in FIG. 3B. In another set of specific, non-limiting embodiments, the carnitine binding site of mCRAT is comprised in a protein structure comprising amino acid residues TRP102, LEU103, ALA106, TYR107, ILE116, TYR117, SER118, SER119, PRO120, GLY121, TYR341, GLU342, HIS343, ALA344, ALA345, ALA346, GLU347, GLY348, PRO349, PRO350, ILE351, TYR452, GLU453, SER454, ALA455, SER456, LEU457, ARG458, ARG464, THR465, ASP466, THR467, ILE468, ARG469, ARG518, LEU551, SER552, THR553, SER554, GLN555, VAL556, MET564, PHE565, PHE566, GLY567, PRO568, and VAL569 and more preferably Arg518, Thr465, Trp102, Tyr107, Glu347, His343, Phe566, Val569, Ser552, Ser454, and Tyr452 (see FIG. 3D), in a configuration as defined by the atomic coordinates set forth in FIG. 7 for unbound mCRAT or in a configuration as defined by the coordinates set forth in FIG. 8 for mCRAT bound to carnitine. In another set of specific, non-limiting embodiments, the CoA binding site of mCRAT is comprised in a protein structure comprising amino acid residues LEU163, LEU168, HIS343, GLU347, GLY348, PRO349, PRO350, LYS419, ASP420, PHE421, PRO422, LYS423, LEU427, SER428, PRO429, ASP430, ALA431, PHE432, ILE433, GLN434, TYR452, GLU453, SER454, ALA455, SER456, LEU457, ARG458, MET459, ARG464, THR465, ASP466, THR467, ILE468, VAL500, GLN501, HIS503, ARG504, THR507, ASP508, ILE511, GLN555, PRO557, TYR578, PRO580, MET581, and GLU582 and more preferably Lys419, Lys423, Asp430 and Glu453 (see FIG. 4A-D).

In another set of specific, non-limiting embodiments, the carnitine binding site of hCRAT is comprised in a protein structure comprising amino acid residues TRP102, LEU103, ALA106, TYR107, ILE116, TYR117, SER118, SER119, PRO120, GLY121, TYR341, GLU342, HIS343, ALA344, ALA345, ALA346, GLU347, GLY348, PHE349, PRO350, ILE351, TYR452, GLU453, SER454, ALA455, SER456, LEU457, ARG458, ARG464, THR465, ASP466, THR467, ILE468, ARG469, ARG518, LEU551, SER552, THR553, SER554, GLN555, VAL556, MET564, PHE565, PHE566, GLY567, PRO568, and VAL569. In another set of specific, non-limiting embodiments, the CoA binding site of hCRAT is comprised in a protein structure comprising amino acid residues LEU163, LEU168, HIS343, GLU347, GLY348, PHE349, PRO350, LYS419, ASP420, PHE421, PRO422, LYS423, LEU427, SER428, PRO429, ASP430, ALA431, PHE432, ILE433, GLN434, TYR452, GLU453, SER454, ALA455, SER456, LEU457, ARG458, MET459, ARG464, THR465, ASP466, THR467, ILE468, VAL500, GLN501, HIS503, ARG504, THR507, ASP508, ILE511, GLN555, PRO557, TYR578, PRO580, MET581, and GLU582.

In specific non-limiting embodiments of the invention, the carnitine and CoA binding sites of CRAT extend within the three-dimensional CRAT structure to a distance within 10 Å.

Where amino acid residues are designated by numbers, for example, in the statement “the carnitine binding site comprises amino acid residues Arg518, Thr465, Trp102, Tyr107, Glu347, His343, Phe566, Val569, Ser552, Ser454, and Tyr452”, the protein having the carnitine binding site is not required to further comprise additional amino acids which make the numbering of the recited residues correct, or to have at least 569 amino acids. Instead, the numbers are used to refer to specific amino acids as present in a structurally defined CRAT to convey the three-dimensional relationship between the residues. Thus, for example, a virtual model of the carnitine binding site may consist only of residues Arg518, Thr465, Trp102, Tyr107, Glu347, His343, Phe566, Val569, Ser552, Ser454, and Tyr452, with their atoms oriented according to the coordinates set forth in FIG. 7 or FIG. 8, without intervening amino acids, and may be referred to as a “molecule” despite the fact that it is only a virtual molecule. Alternatively, additional atoms may be comprised in the site.

### DESCRIPTION OF THI FIGUTRES

The present invention will now be described by way of the following detailed description of illustrative embodiments thereof in conjunction with the drawings in which

FIG. 1 is a sequence alignment of mouse carnitine acetyltransferase (CRAT) and human liver- and muscle-type carnitine palmitoyltransferase I (L-CPT-I and M-CPT-I);

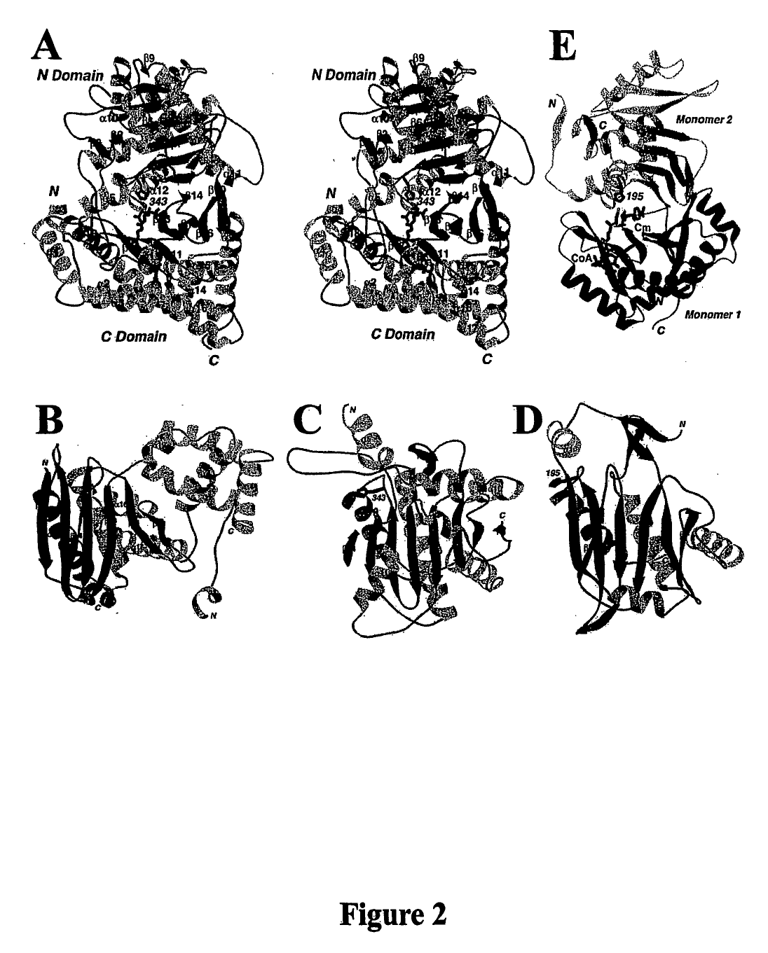

FIG. 2(A-E): (A) is a stereo diagram showing a schematic representation of the structure of CRAT with the β-strands and α-helices labeled and the catalytic His343 residue, carnitine and CoA shown, (B) is a schematic representation of the structure of the C-terminal domain of CRAT, (C) is a schematic representation of the structure of the N-terminal domain of CRAT, shown in the same orientation as the C-terminal domain, (D) is a schematic representation of the structure of the monomer of chloramphenicol acetyltransferase (CAT), viewed in the same orientation as B, with the catalytic His 195 residue shown, (E) is a schematic representation of the structure of two monomers in the trimer of CAT, showing the active site located at the interface between the two monomers and the substrates CoA and chloramphenicol (Cm) shown;

FIG. 3(A-D) is a schematic diagram showing the carnitine binding site of CRAT where (A) shows the final 2Fₒ-F_{c} electron density map for carnitine at 1.9 Å resolution with a contour level of 1 σ, (B) is a stereo diagram showing the carnitine binding site of CRAT with the side chain of the catalytic His343 residue and carnitine shown, (C) is a molecular surface representation of CRAT in the region of the carnitine binding site, and (D) is a schematic representation of the interactions between carnitine and CRAT;

FIG. 4(A-D) shows the CoA binding site of CRAT where (A) shows the final 2Fₒ-F_{c} electron density map for CoA at 2.3 Å resolution with a contour level of 1, (B) is a stereo diagram showing the CoA binding site of CRAT, (C) shows the overlap of the binding modes of CoA to CRAT and CAT, and (D) is a molecular surface representation of CRAT in the region of the CoA binding site;

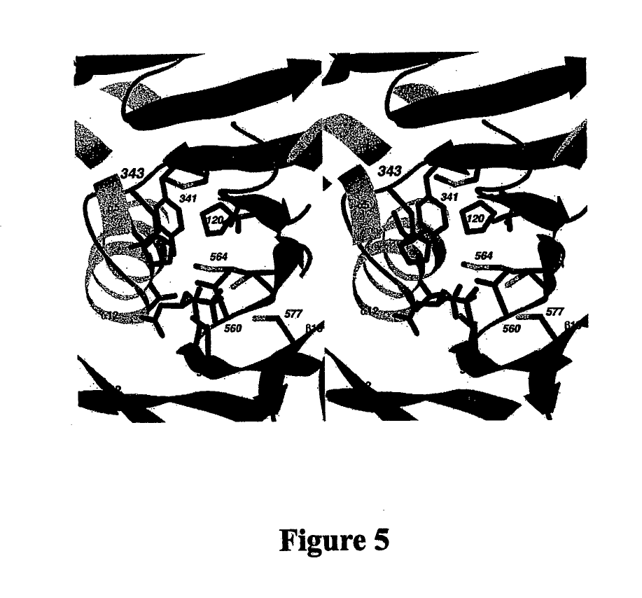

FIG. 5 is a model for the binding mode of acetylcarnitine indicating that the acetyl group points towards a hydrophobic pocket that could also be used to bind long chain acyl groups;

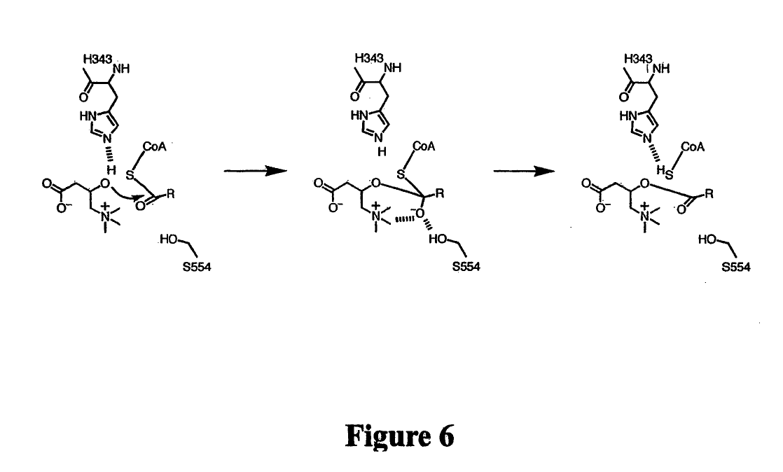

FIG. 6 shows the catalytic mechanism of carnitine transferases indicating that the catalytic His343 residue can extract the proton from either carnitine or CoA and the oxyanion in the tetrahedral intermediate is stabilized by interactions with carnitine and the side chain hydroxyl of Ser554;

FIG. 7 lists the atomic coordinates of free mCRAT of the present invention;

FIG. 8 lists the atomic coordinates of the mCRAT:carnitine complex of the present invention;

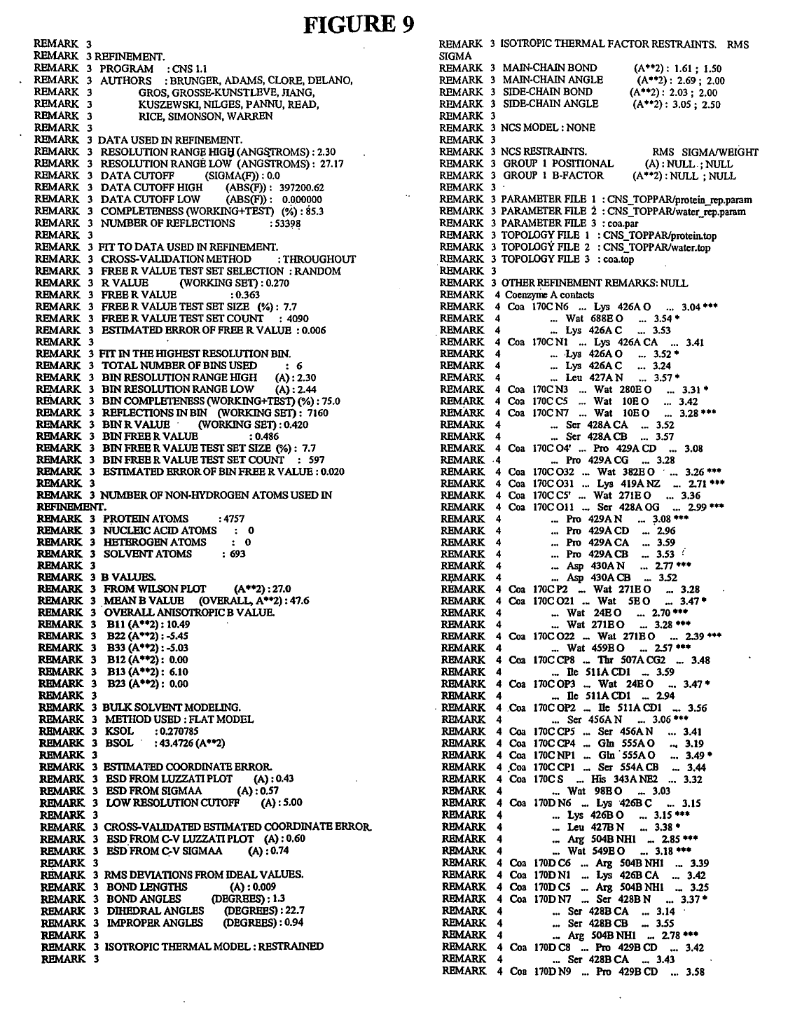

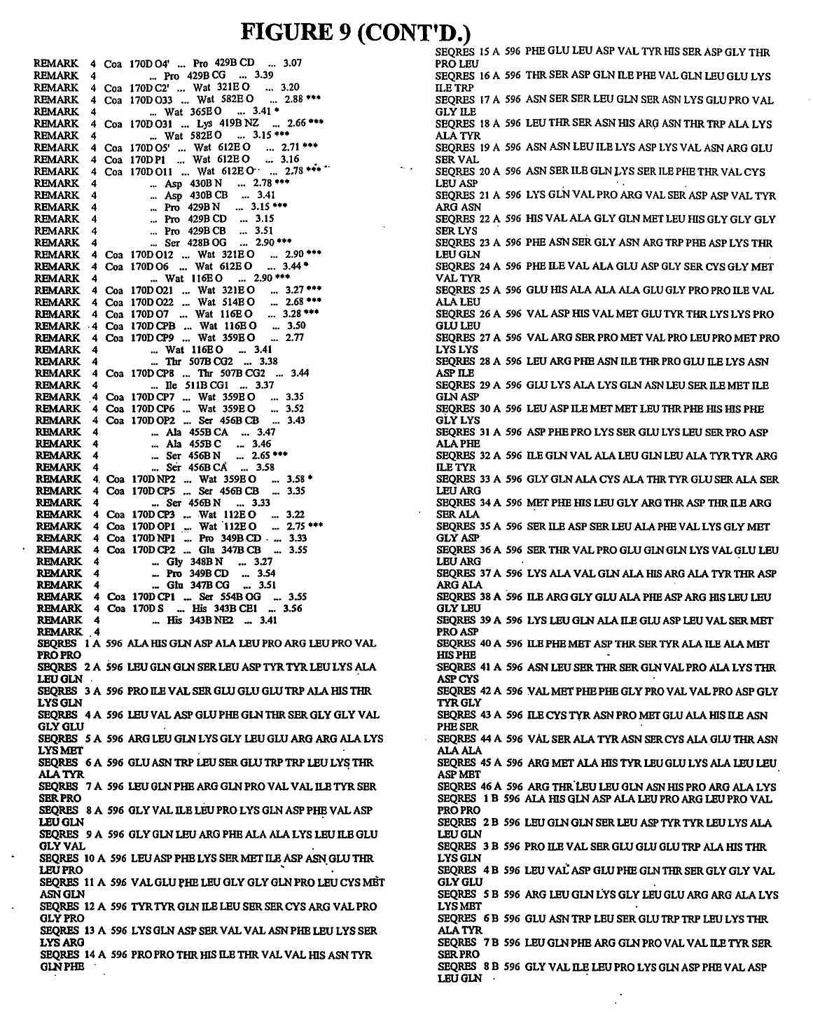

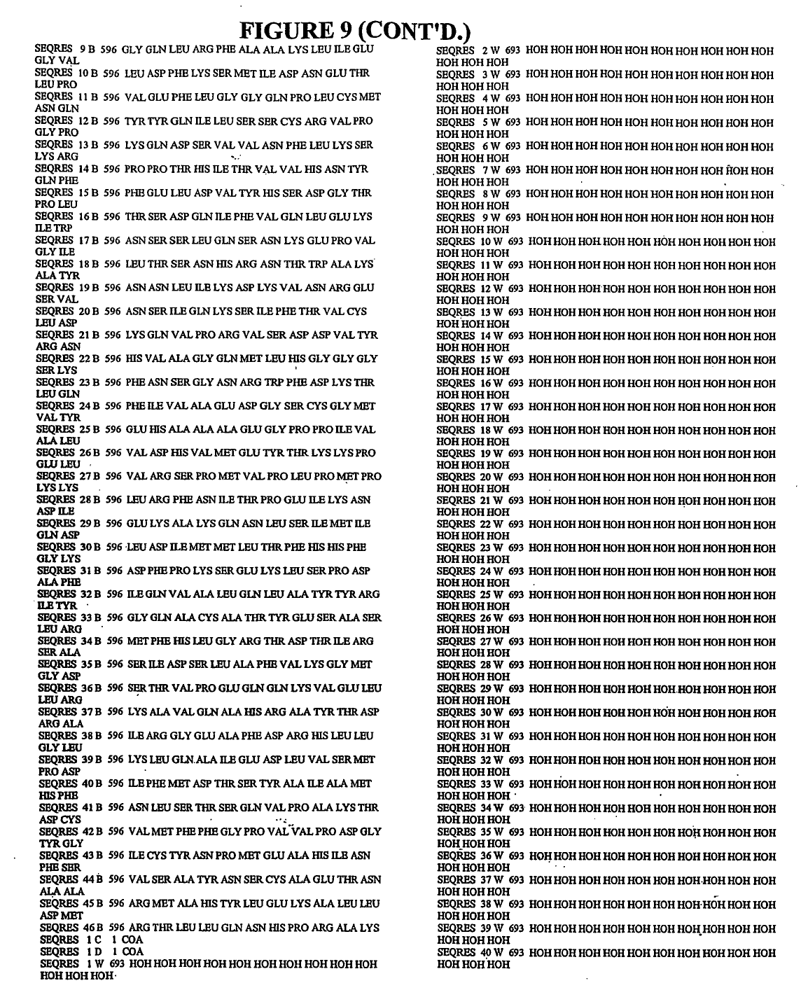

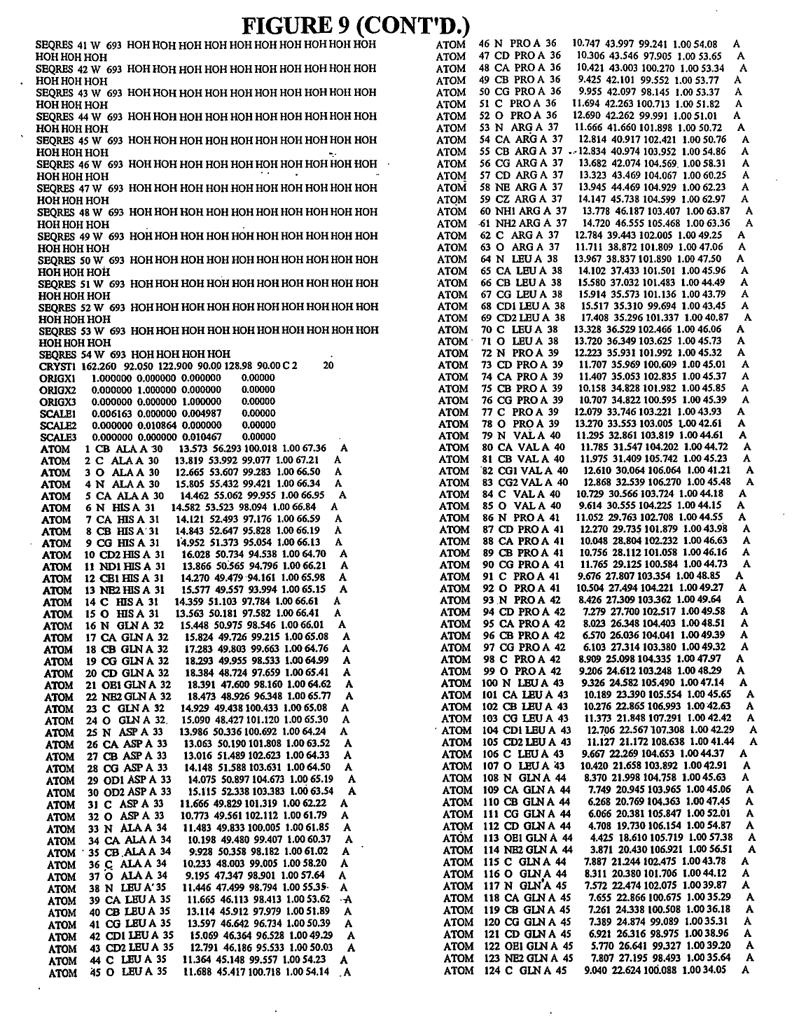

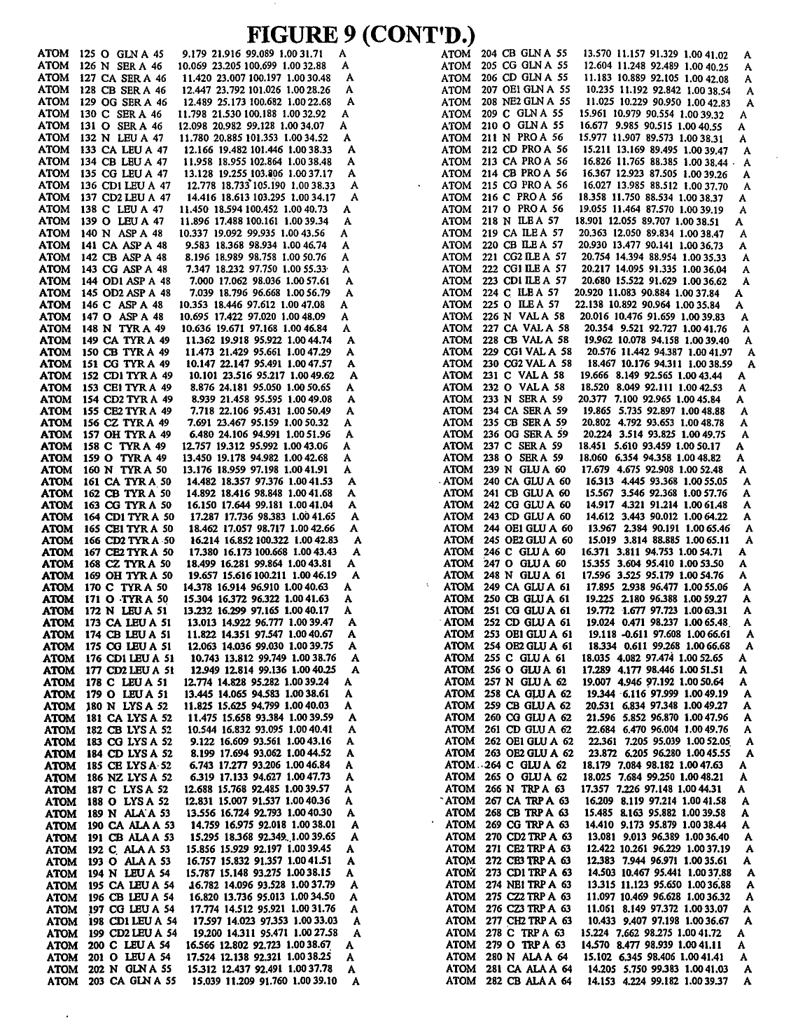

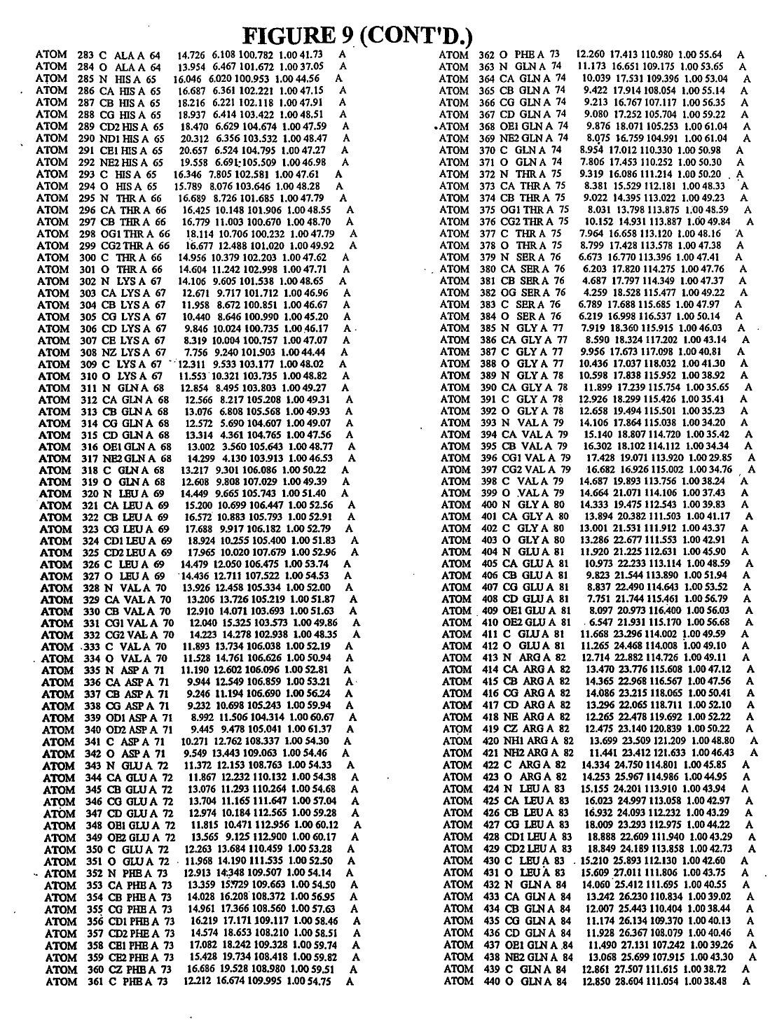

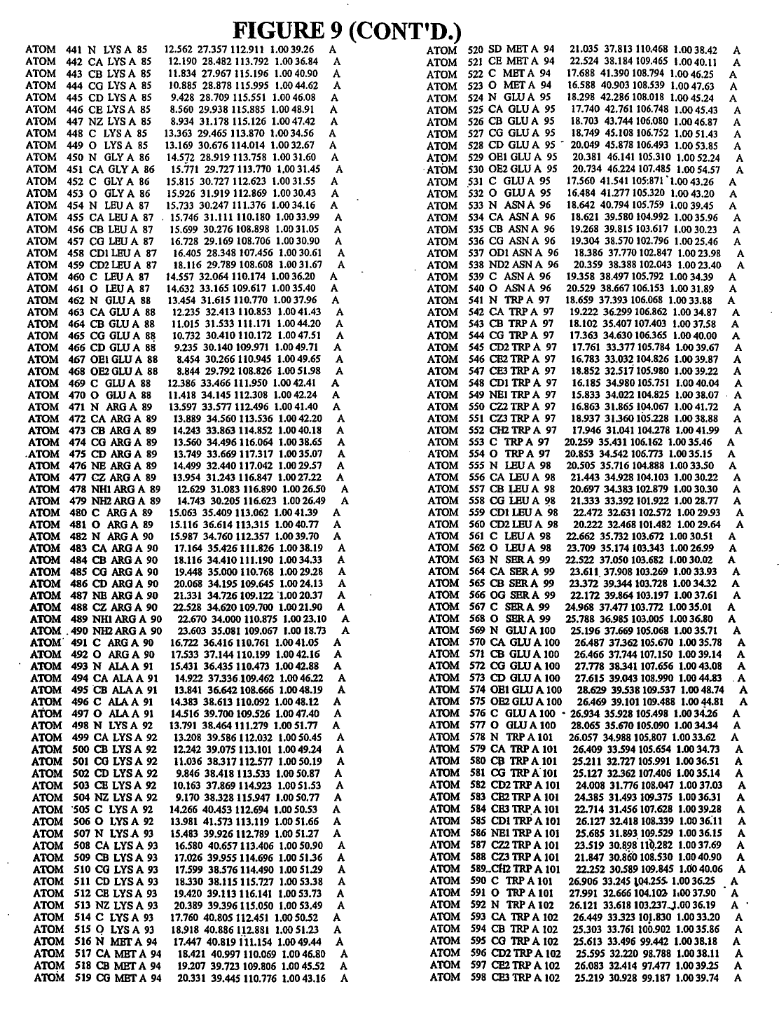

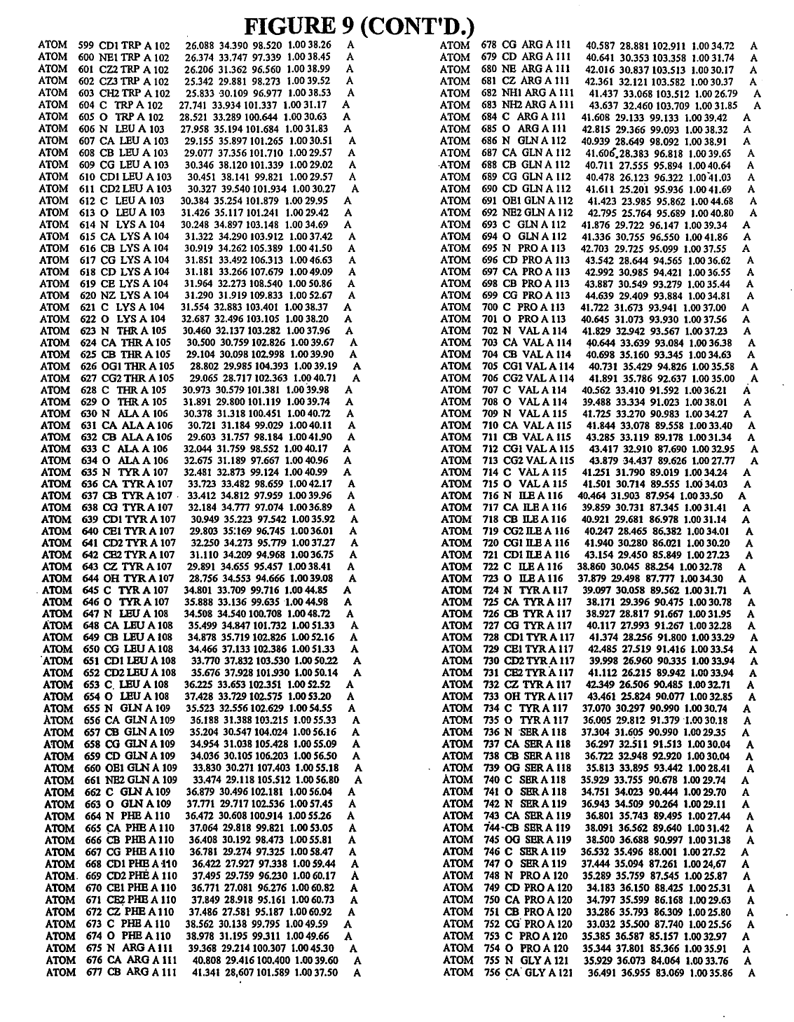

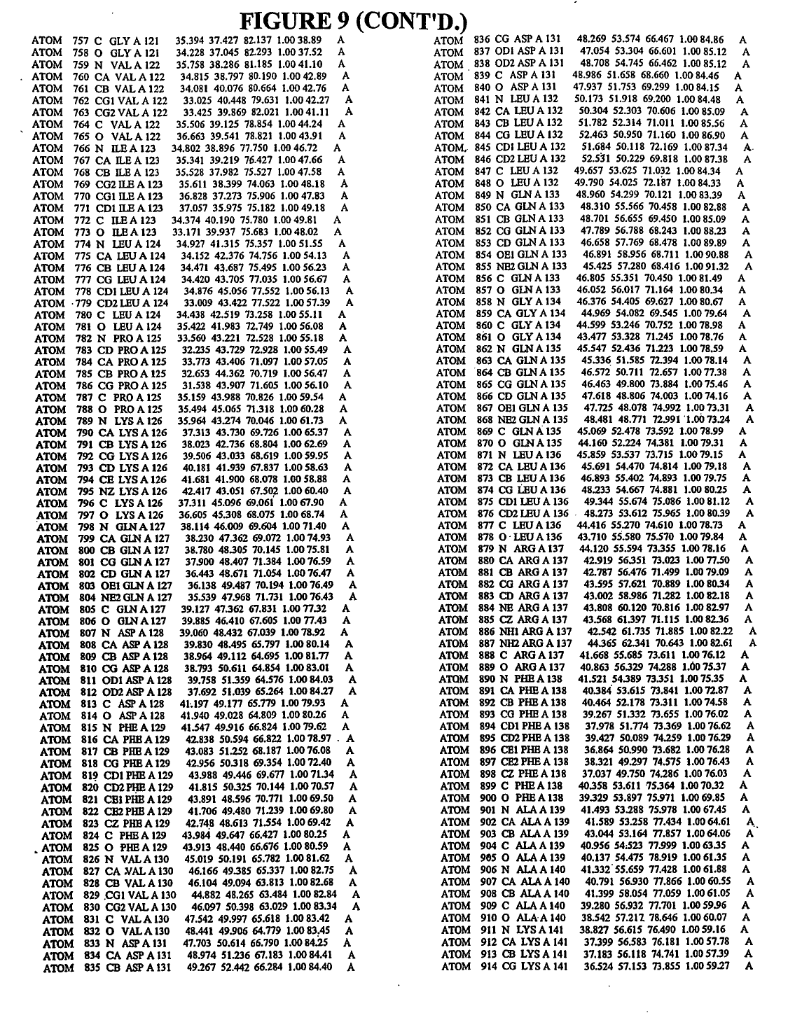

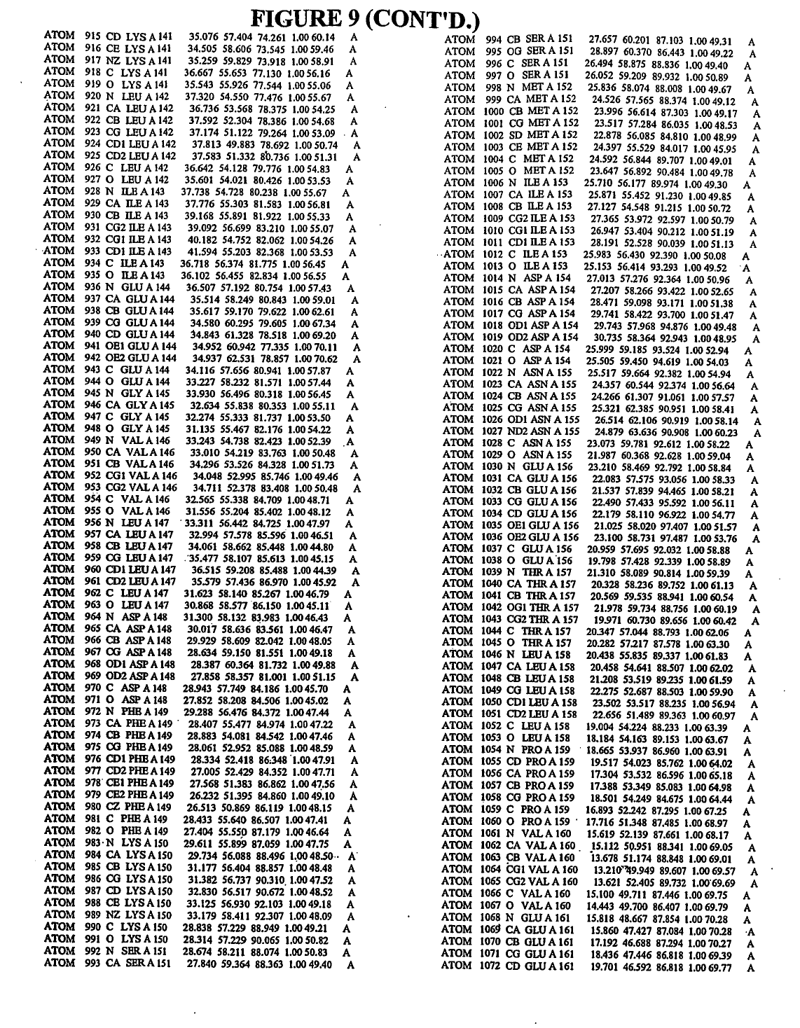

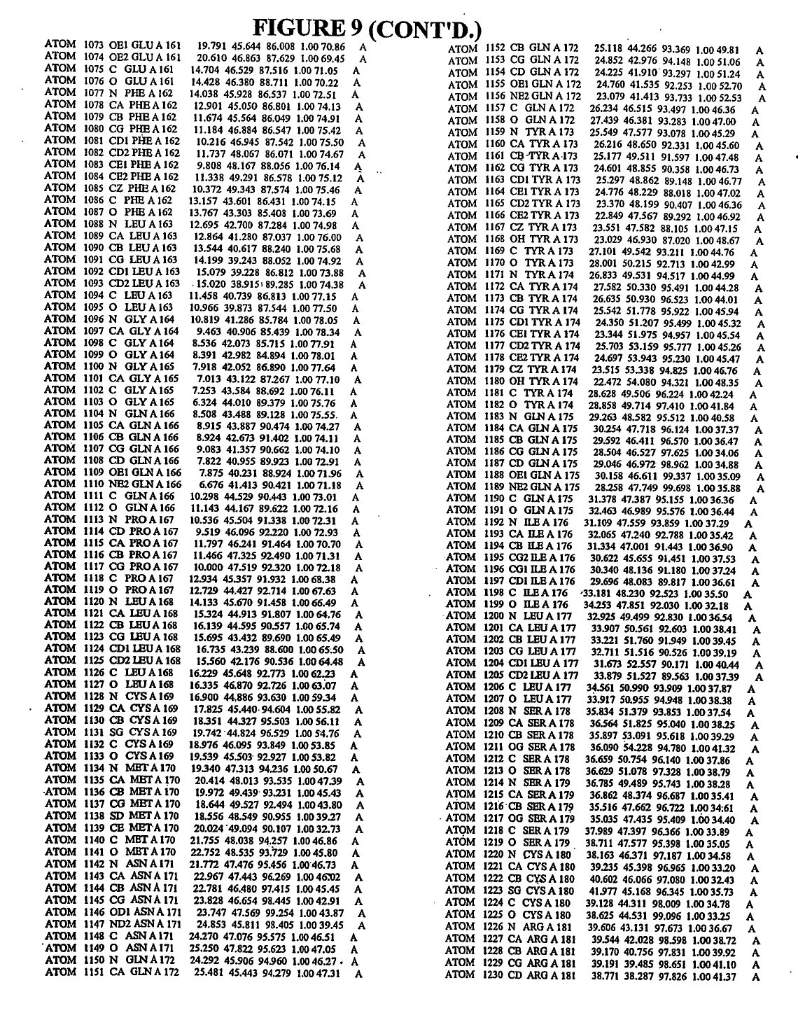

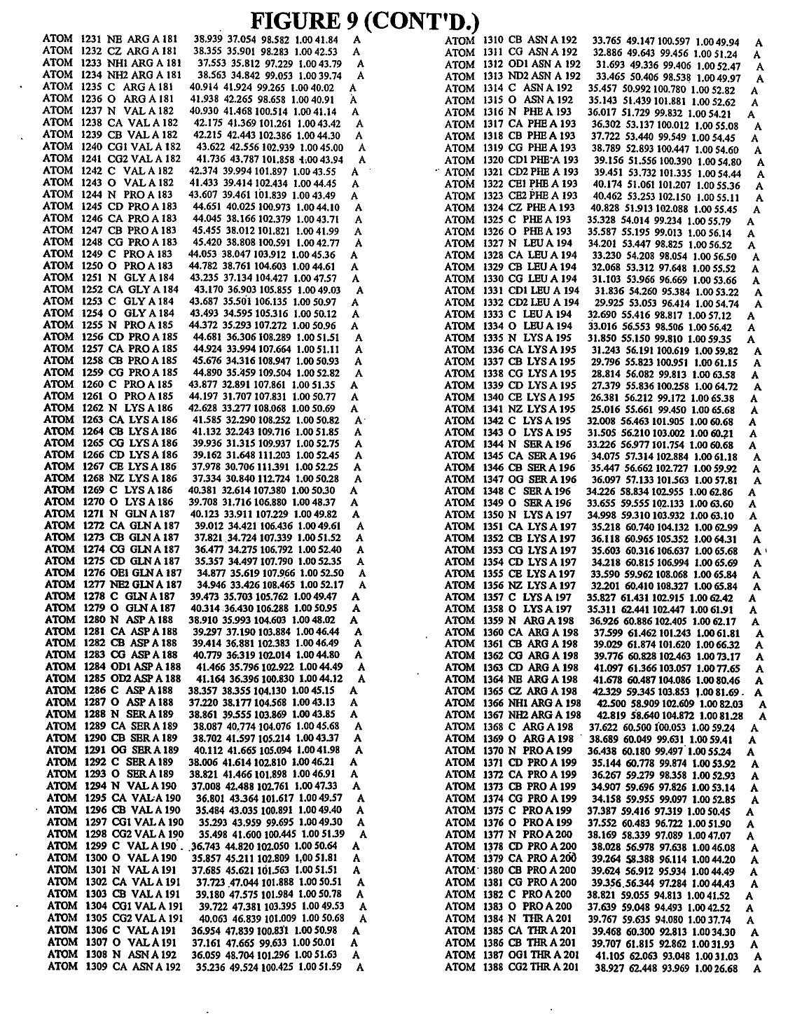

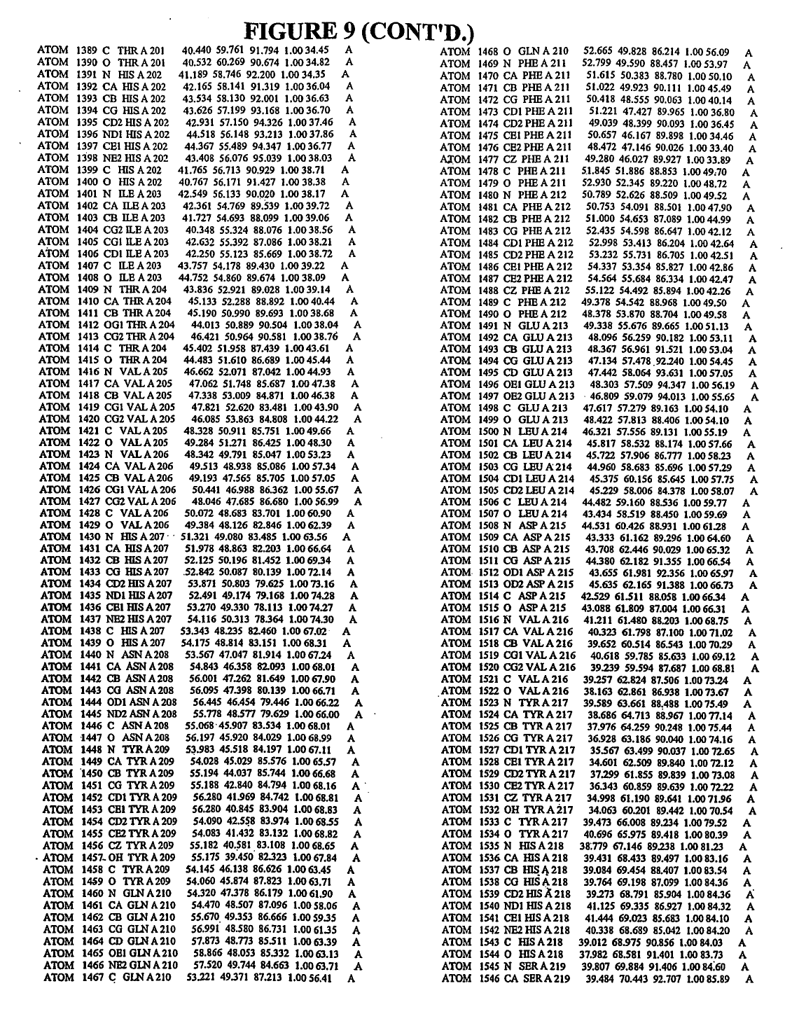

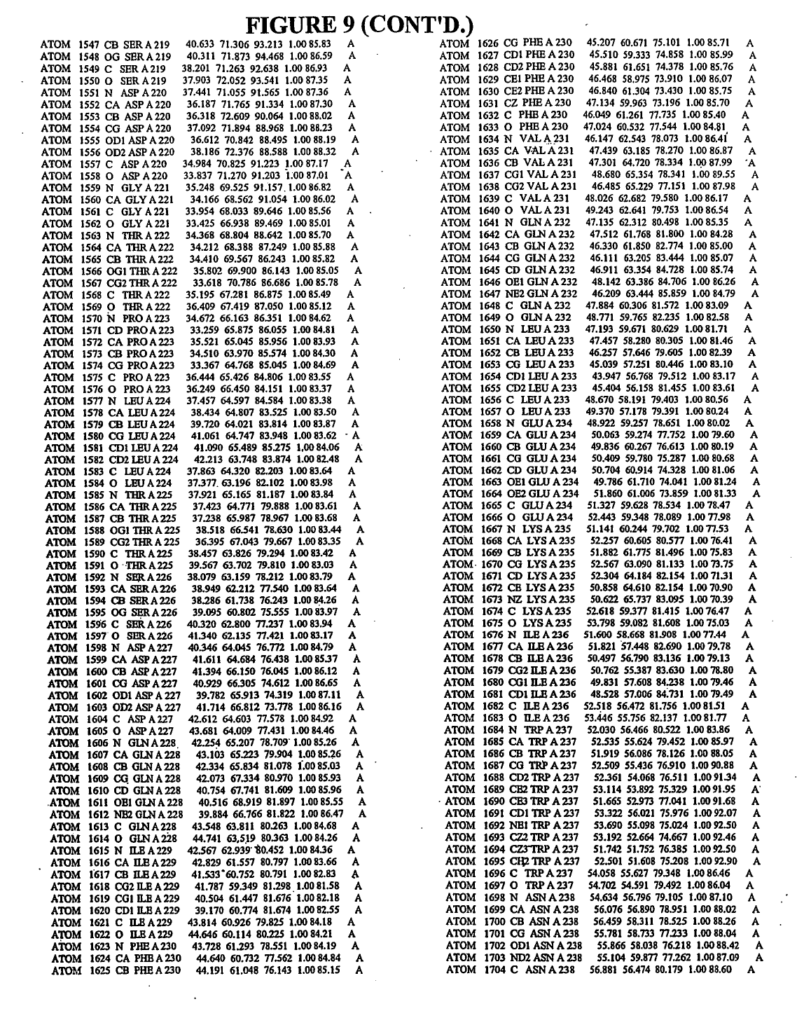

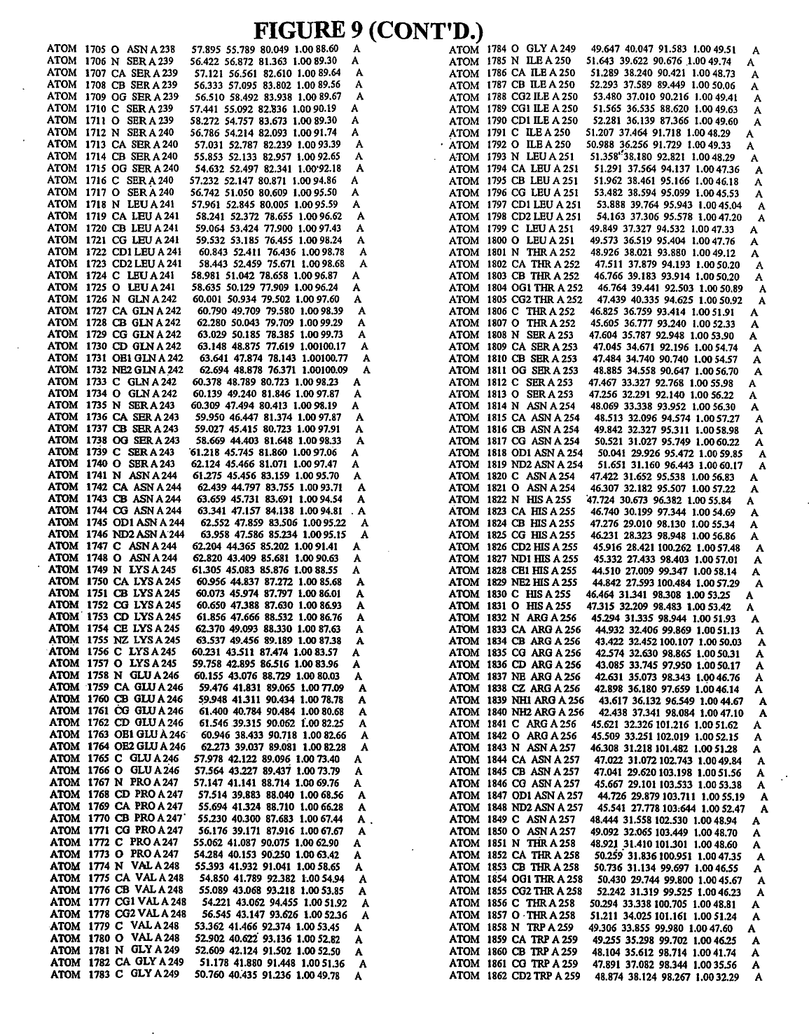

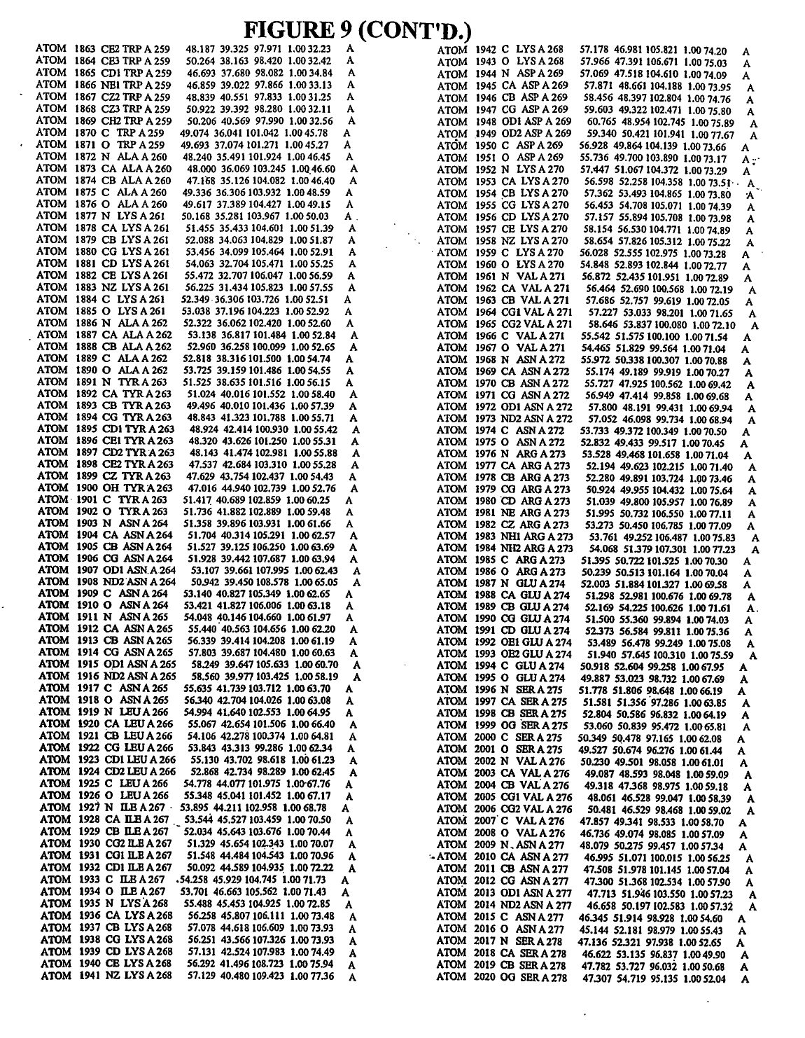

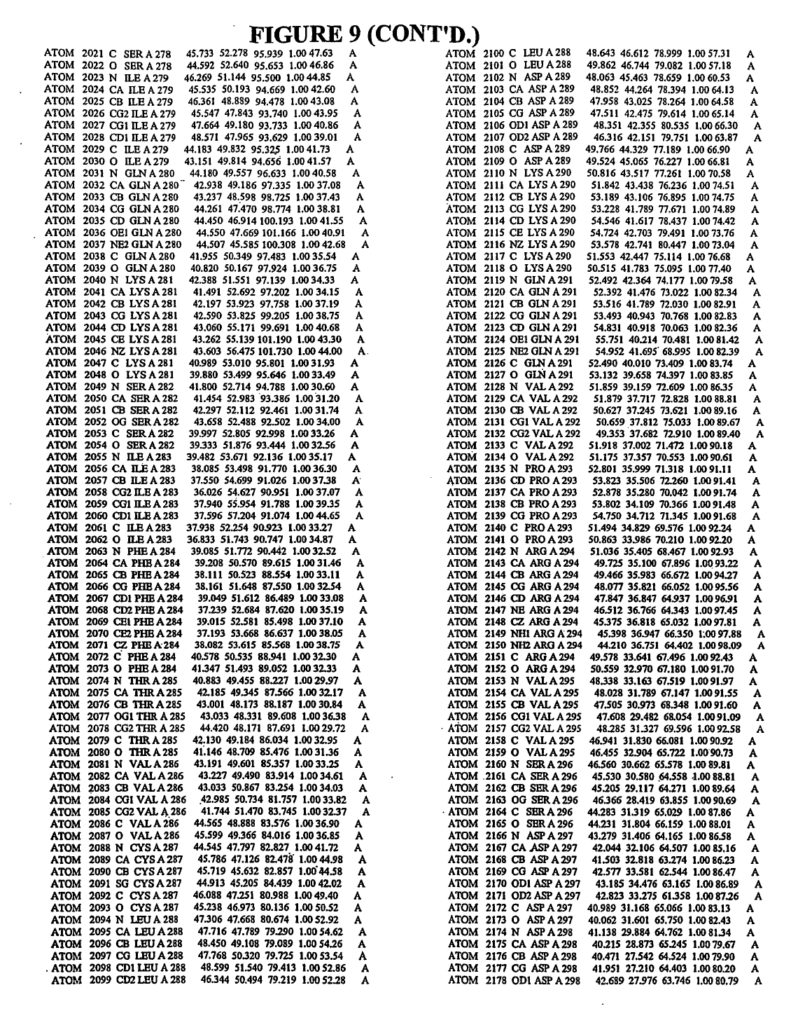

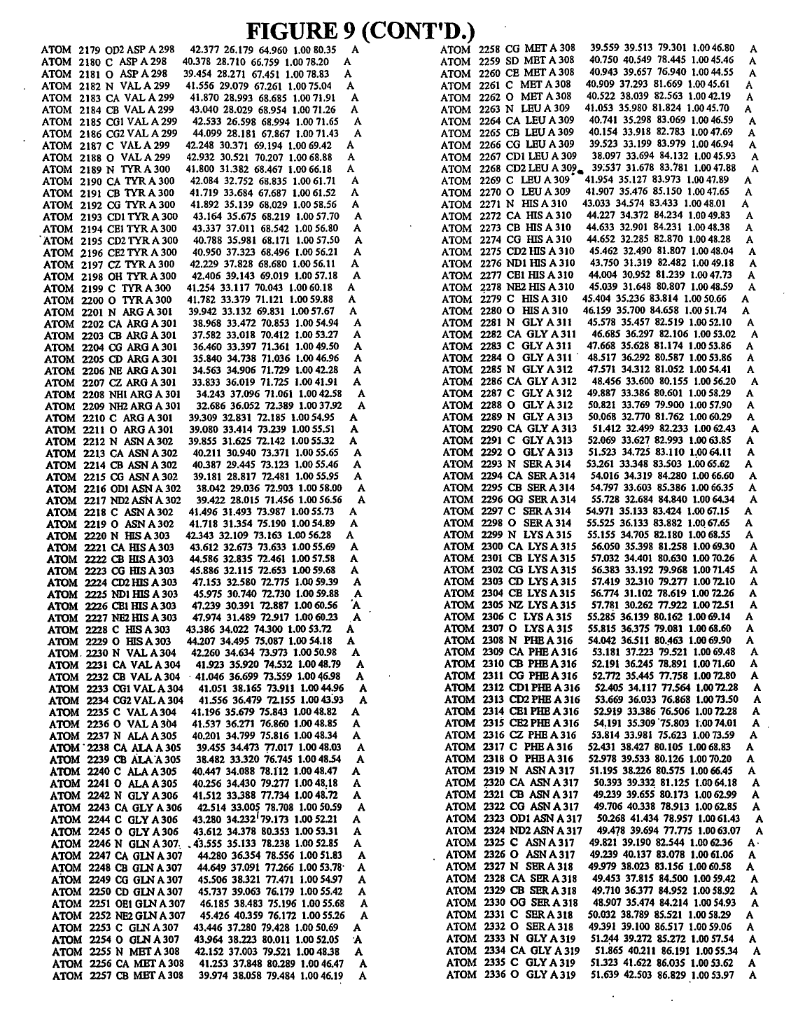

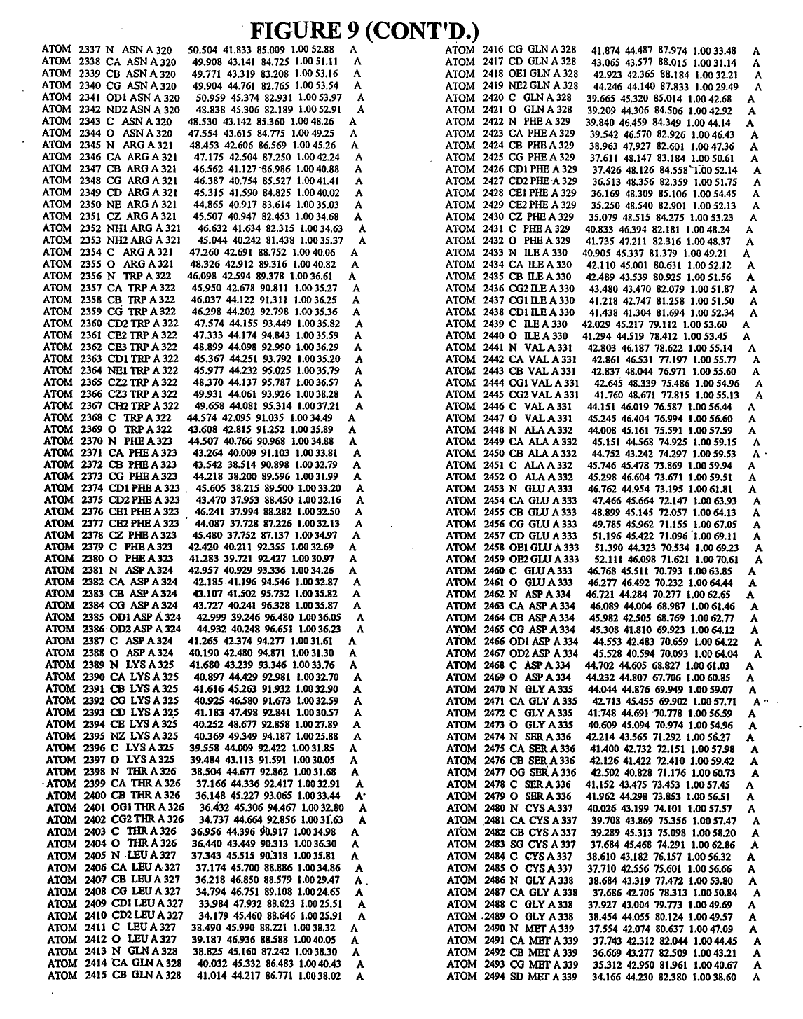

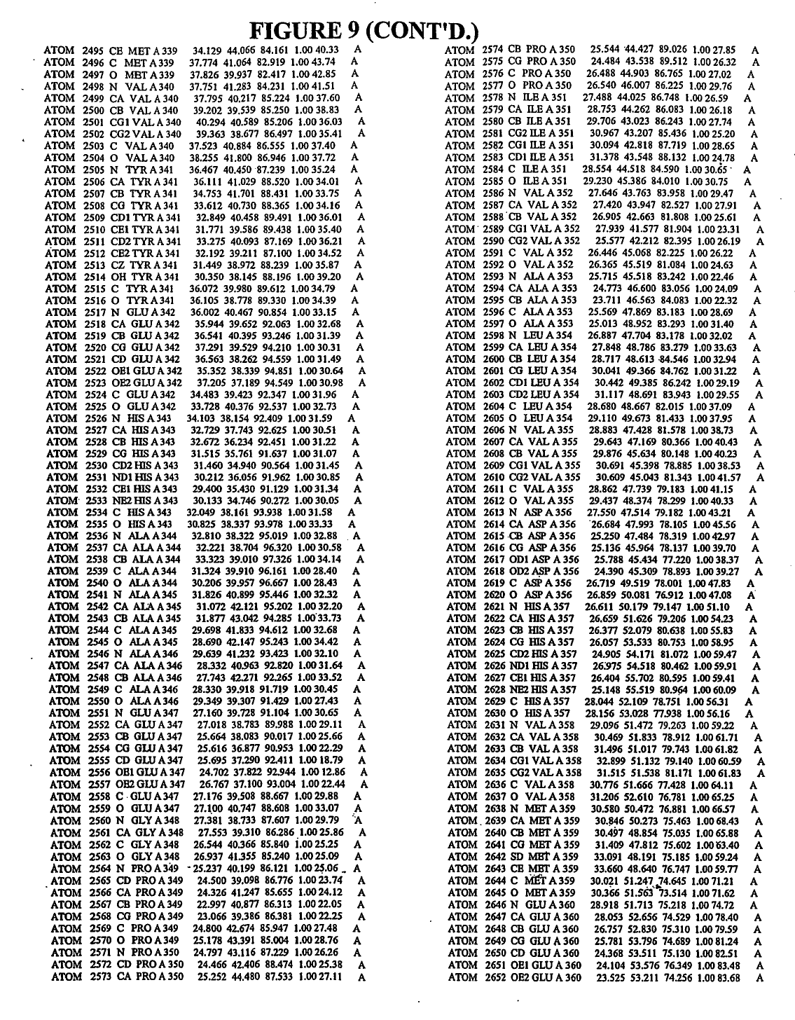

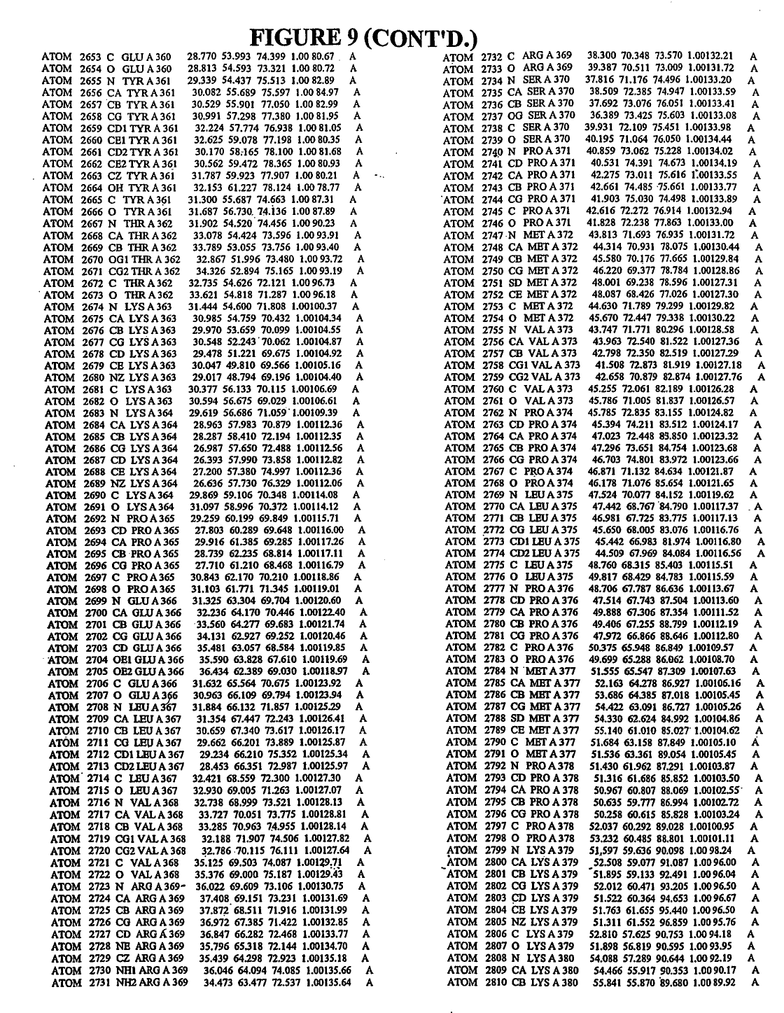

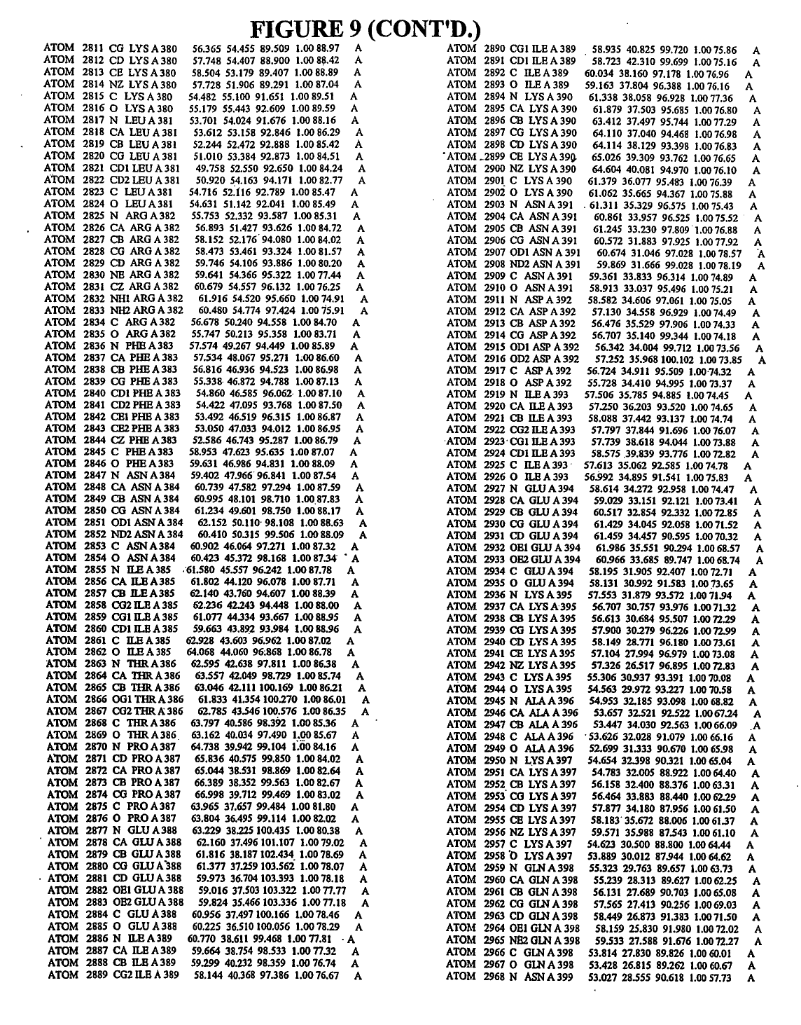

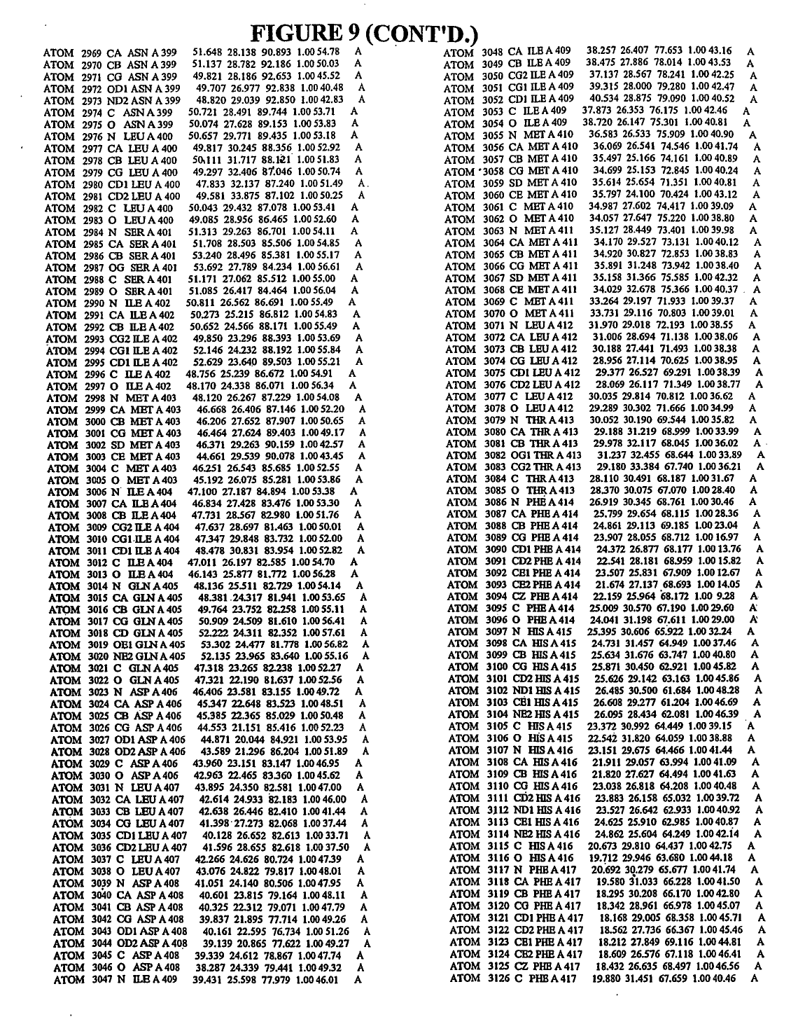

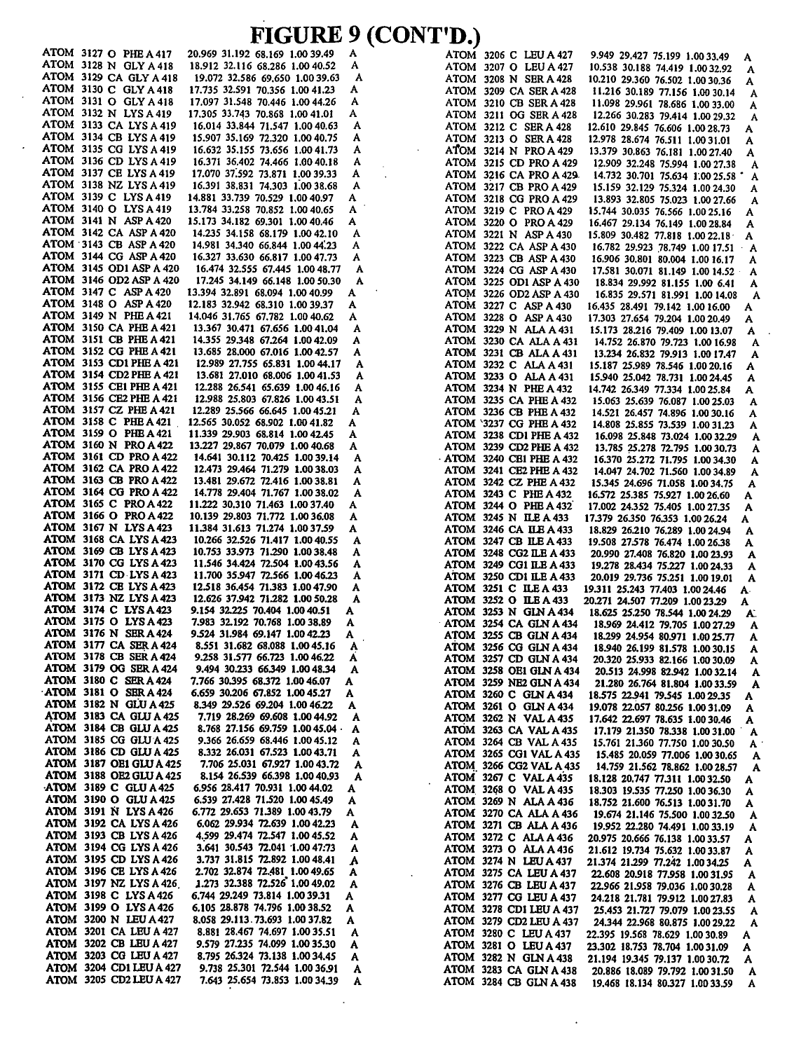

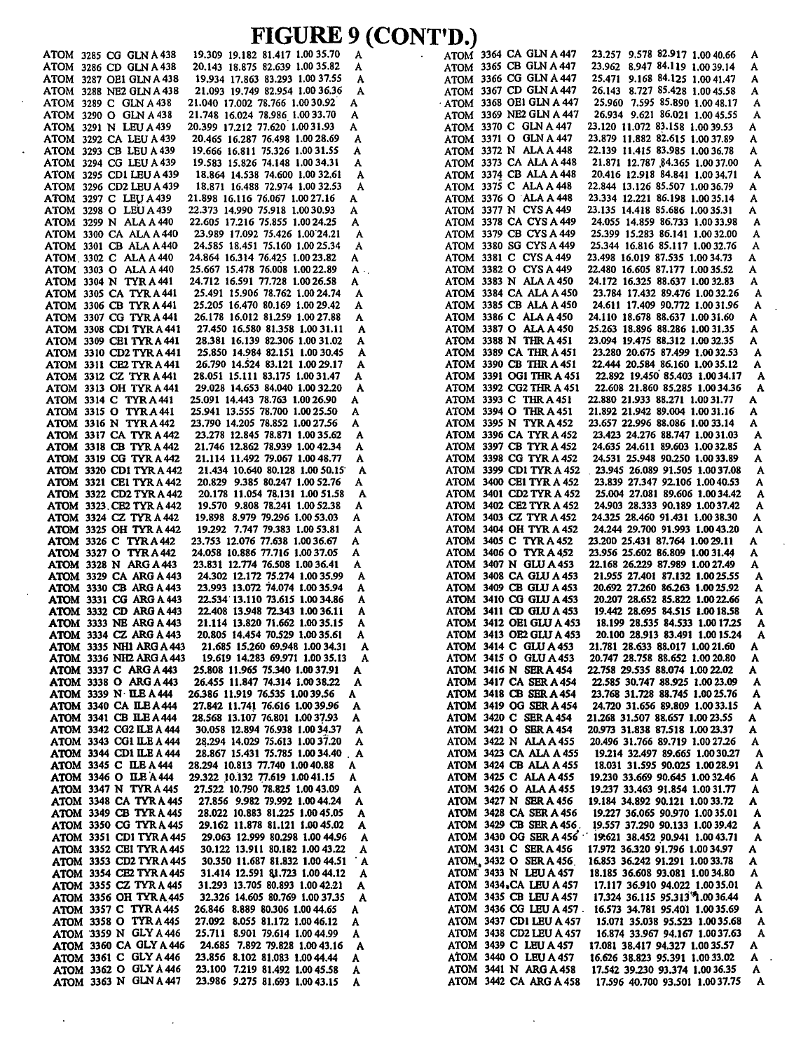

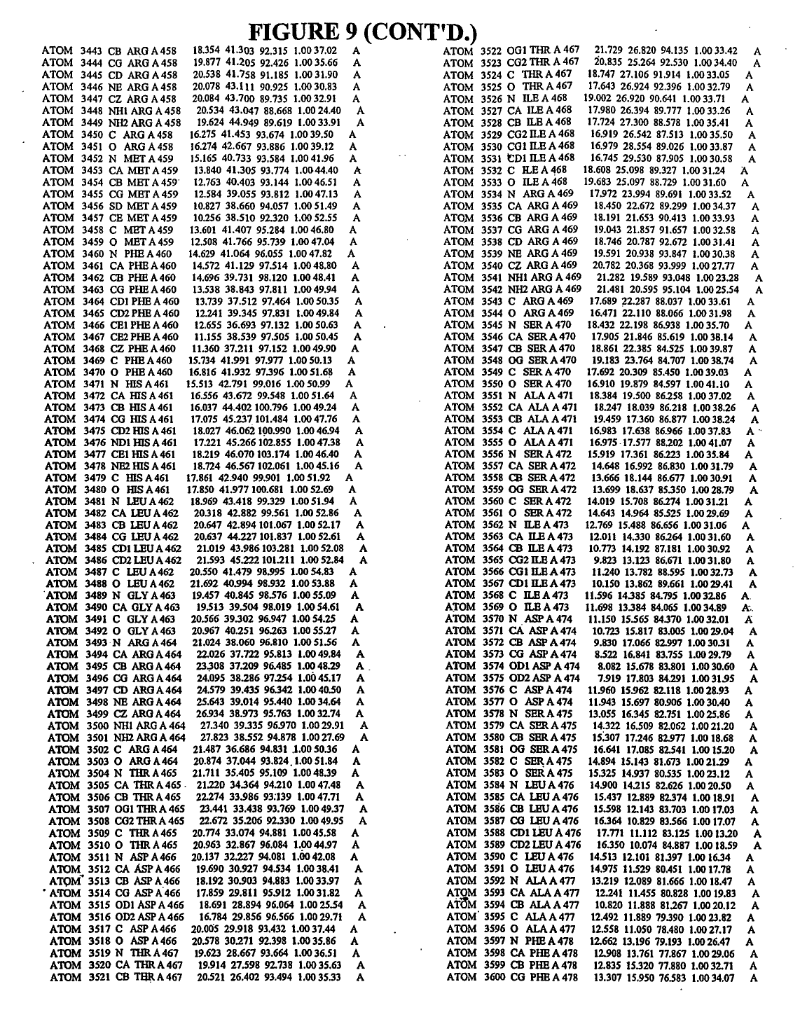

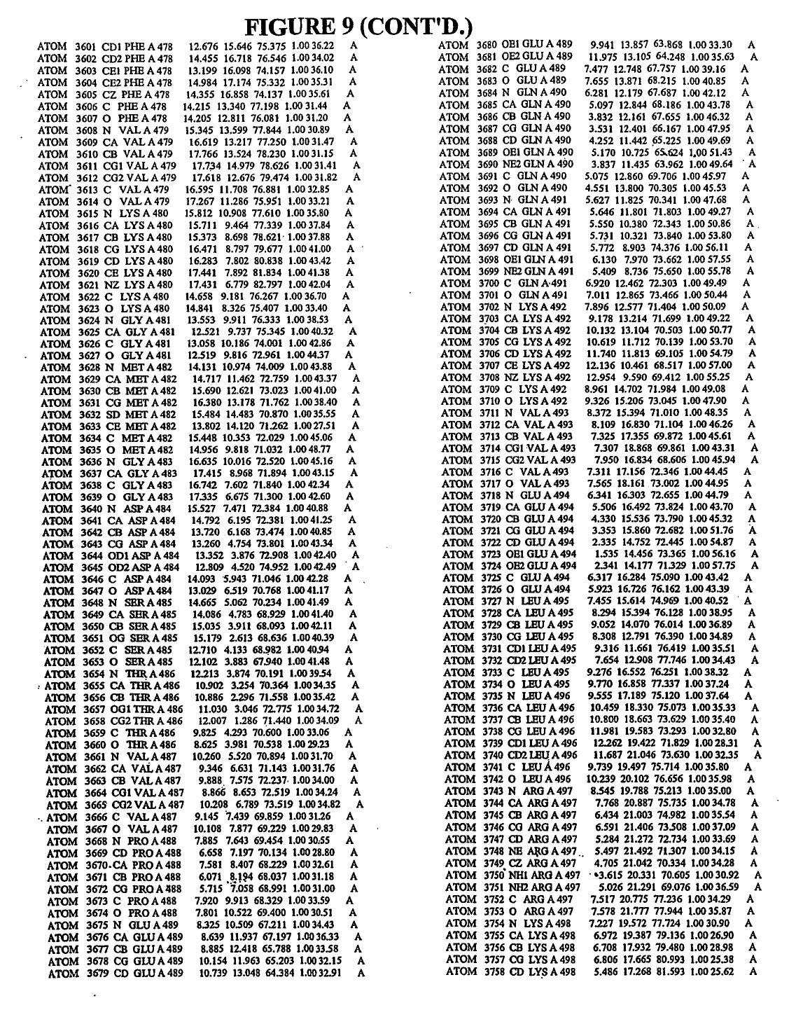

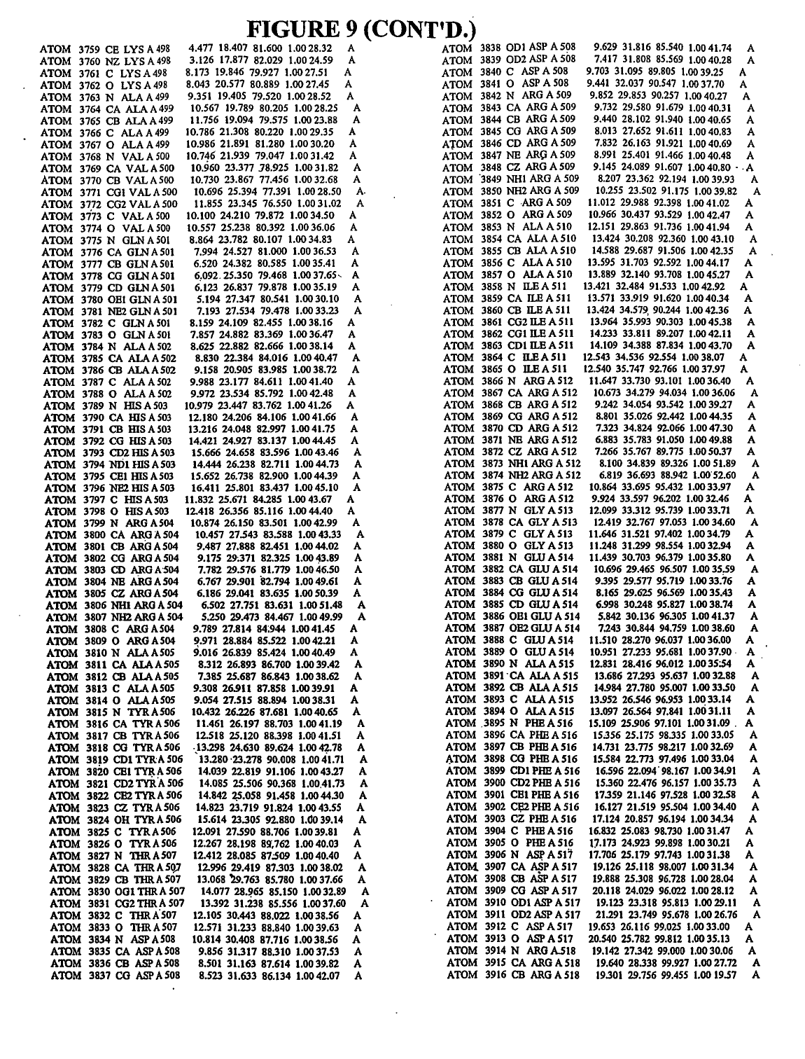

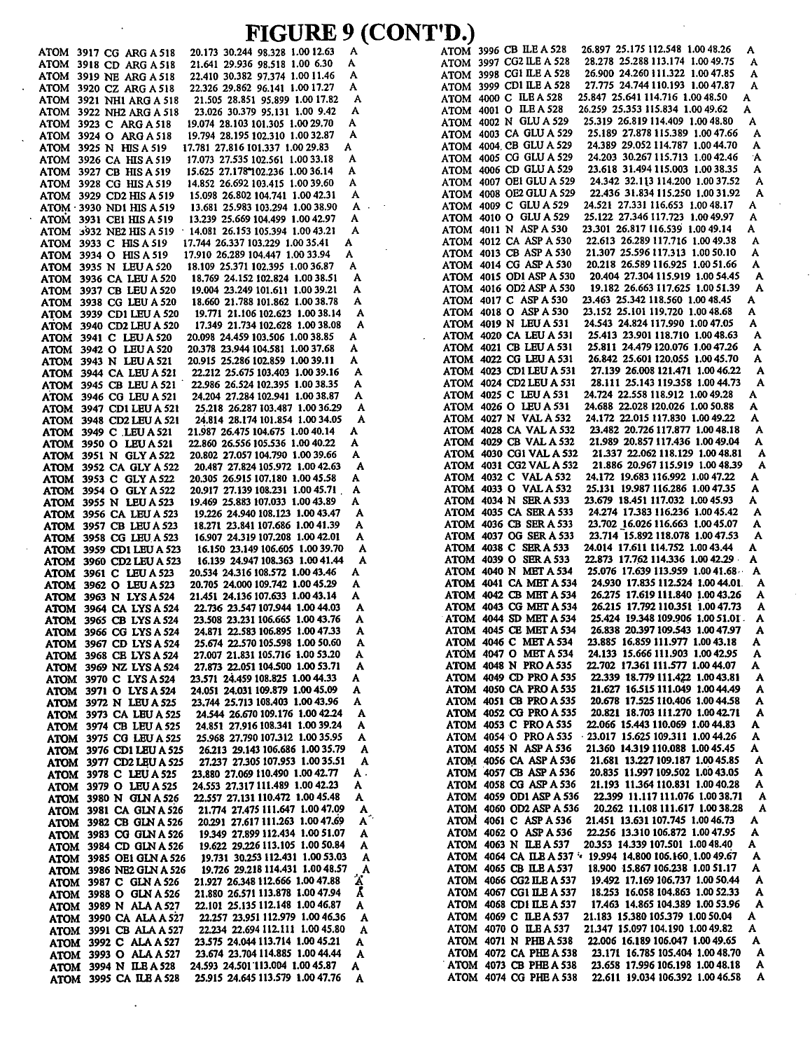

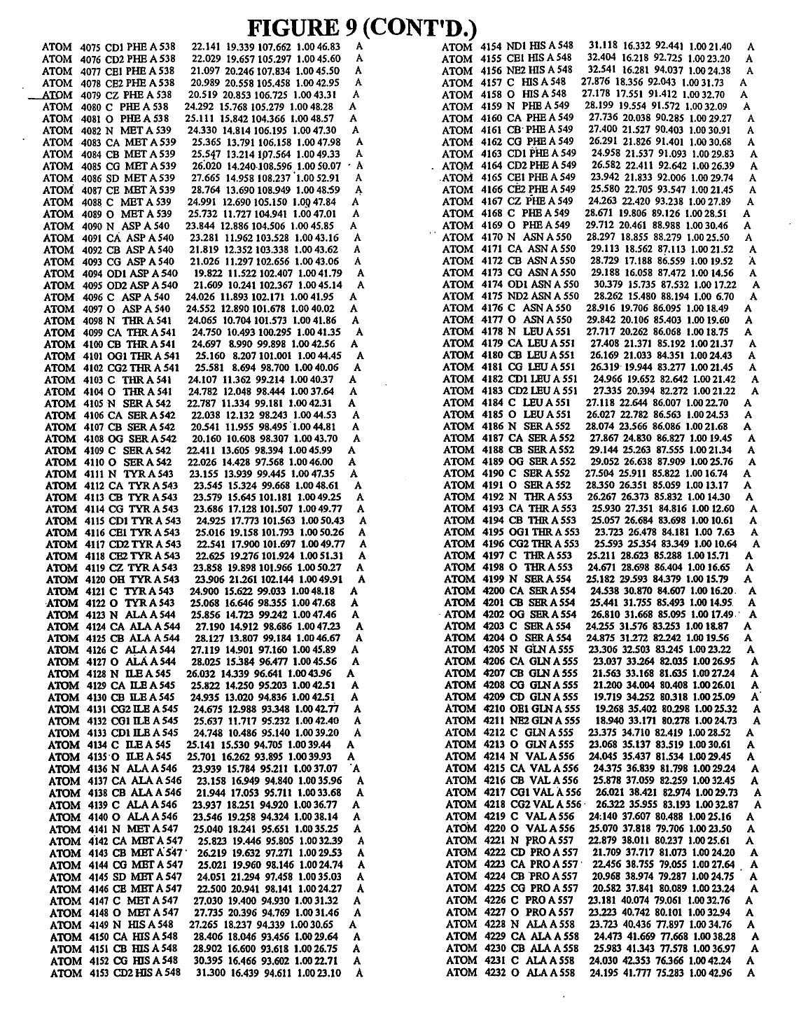

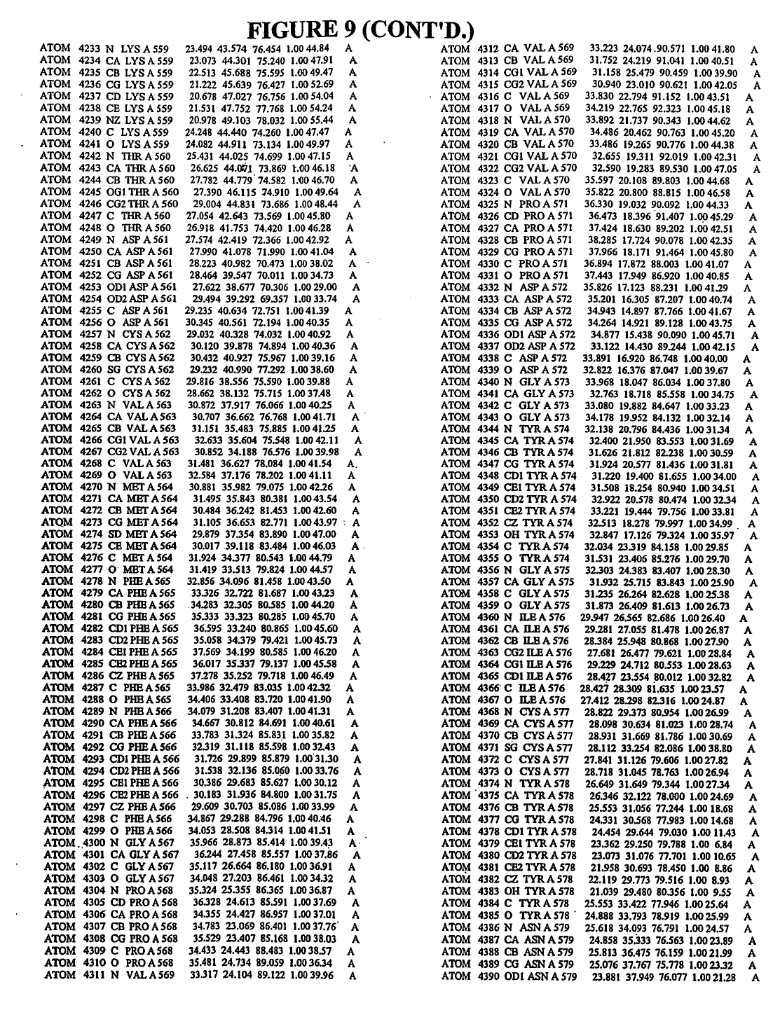

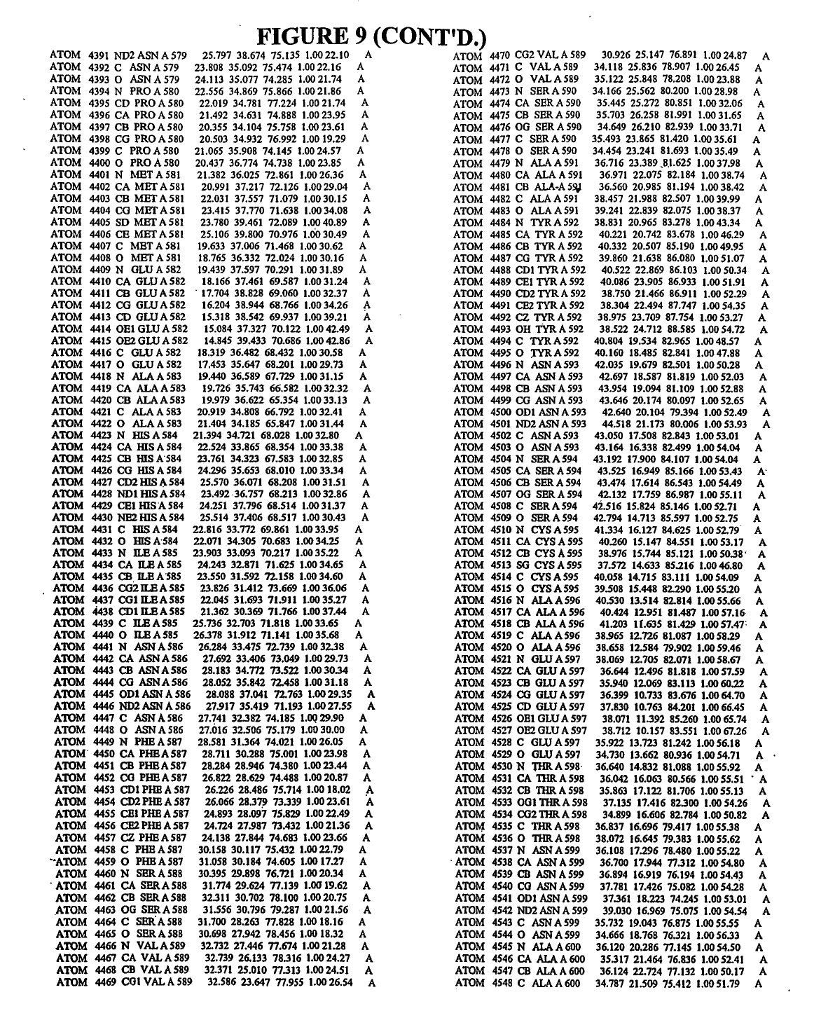

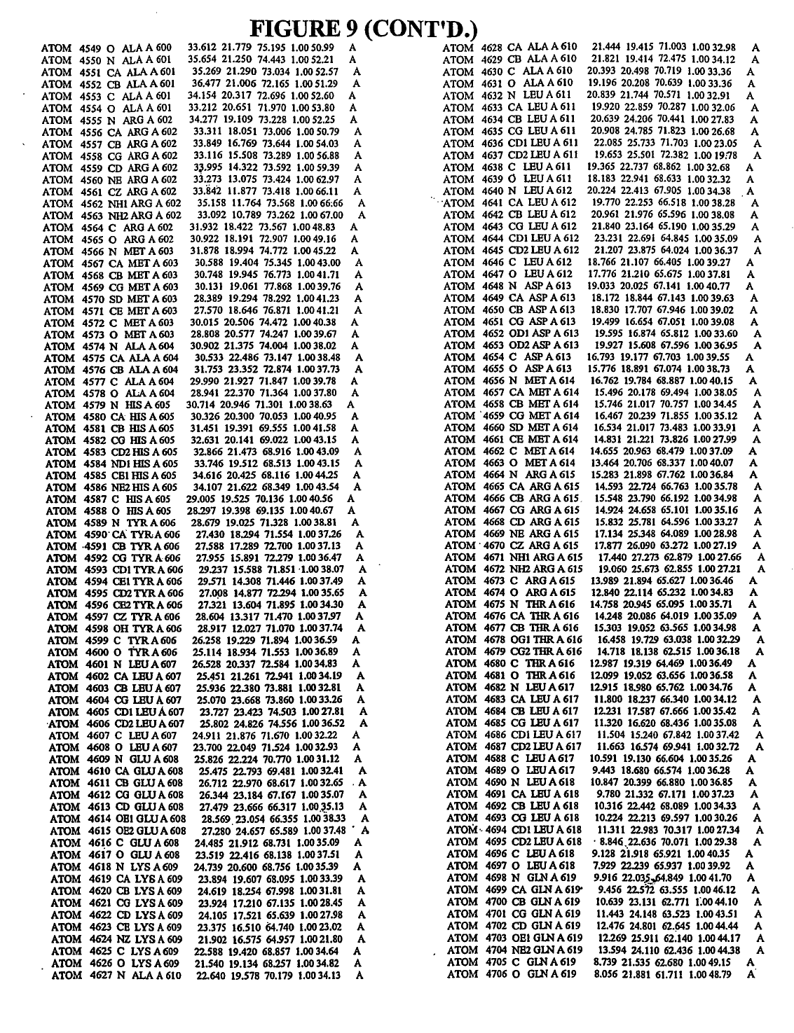

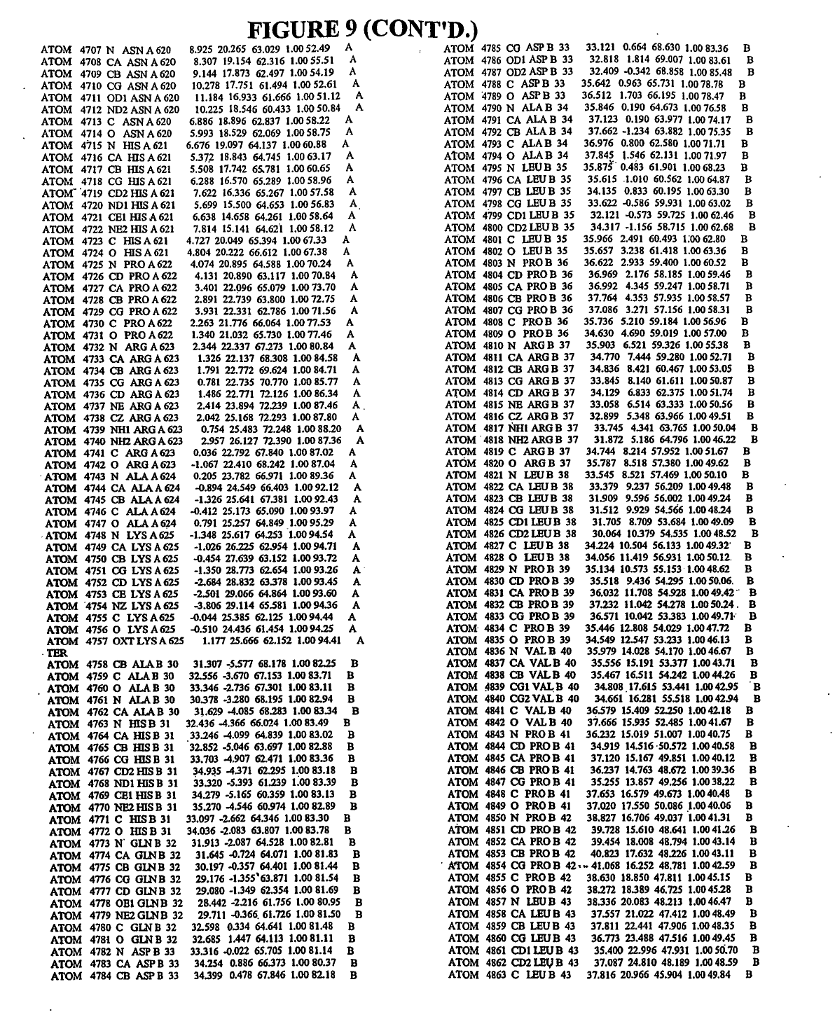

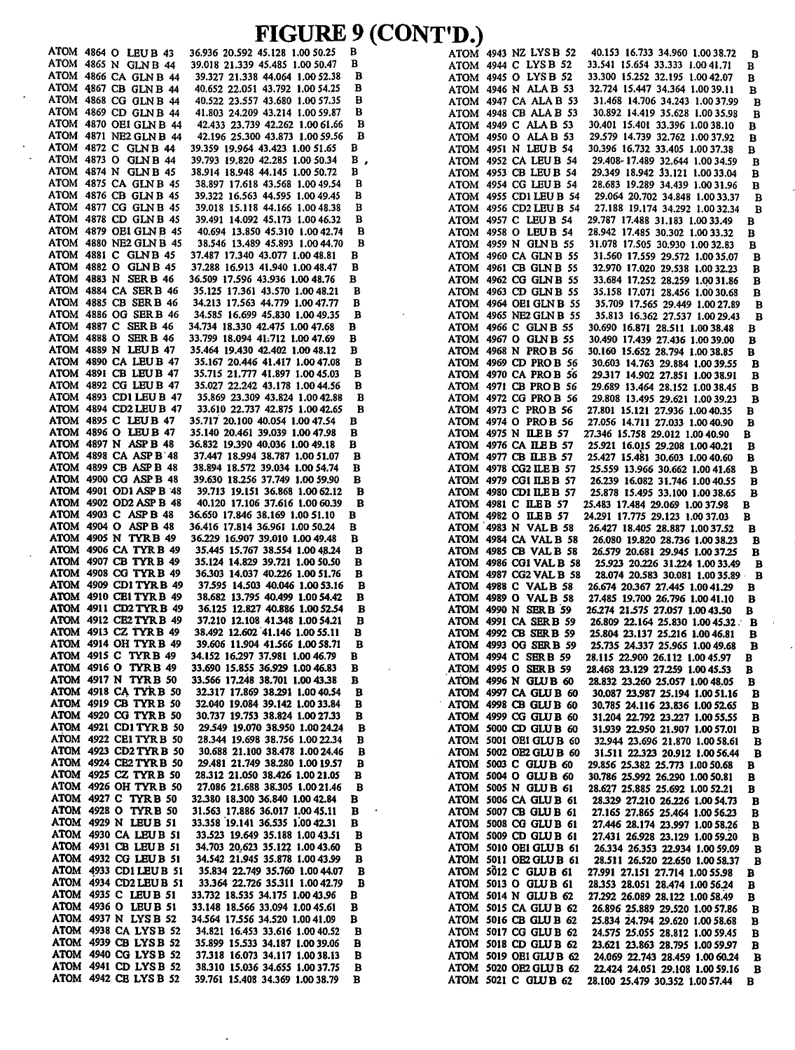

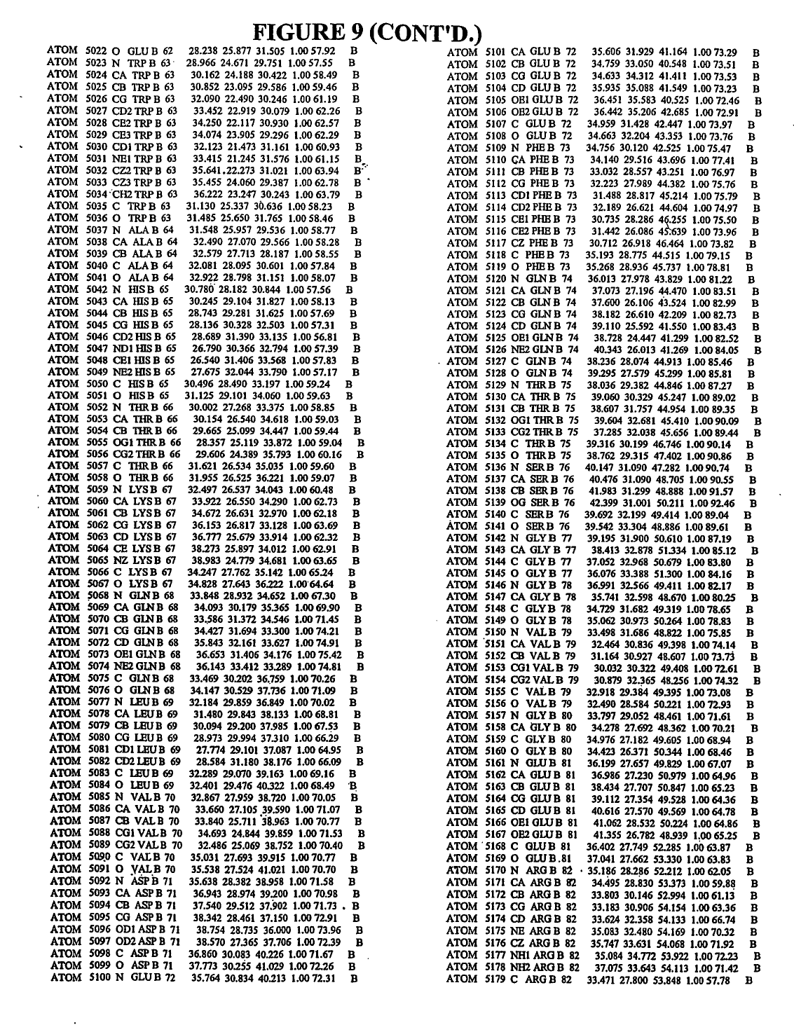

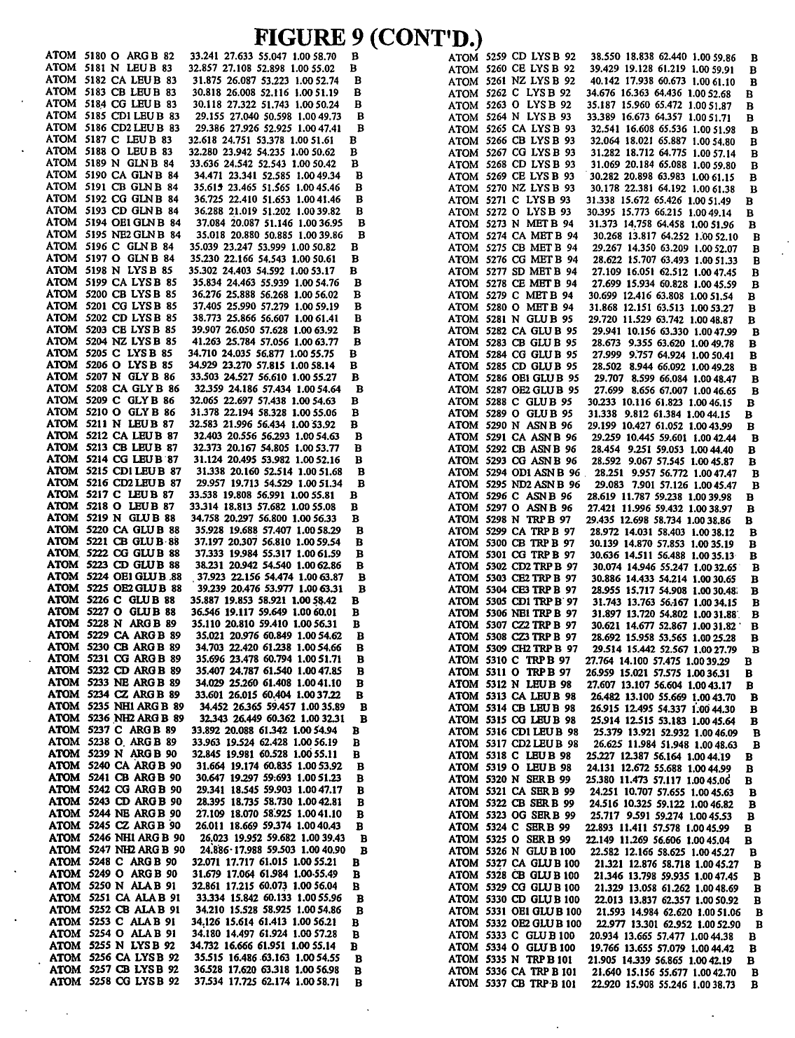

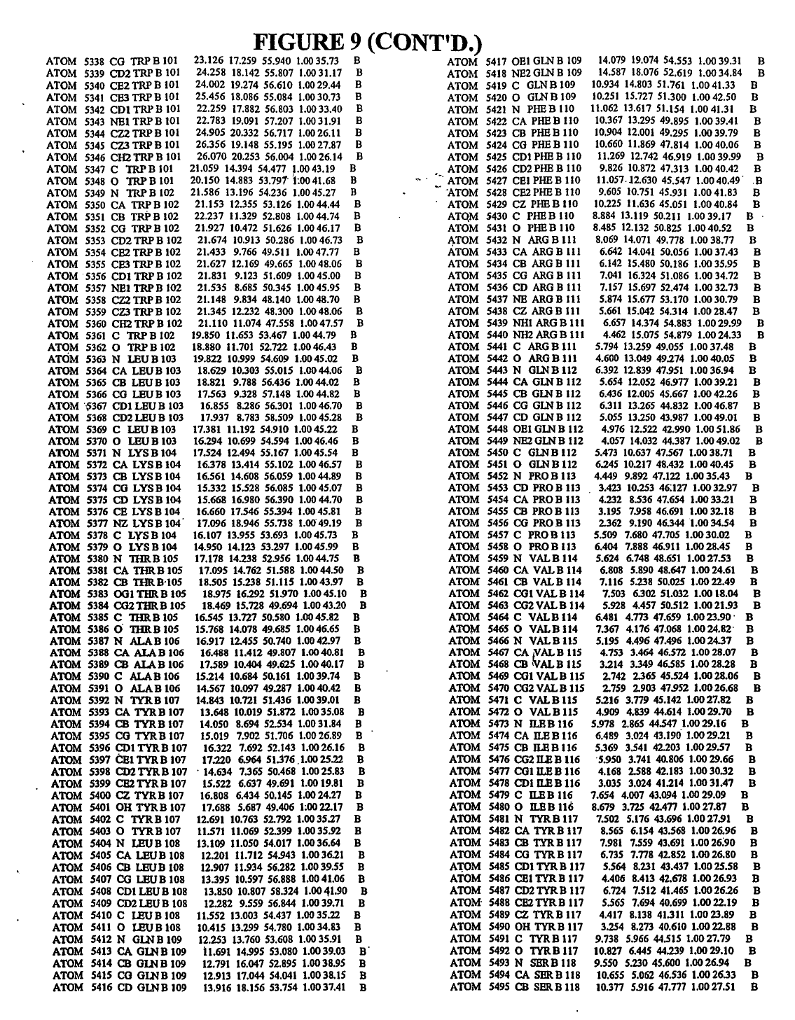

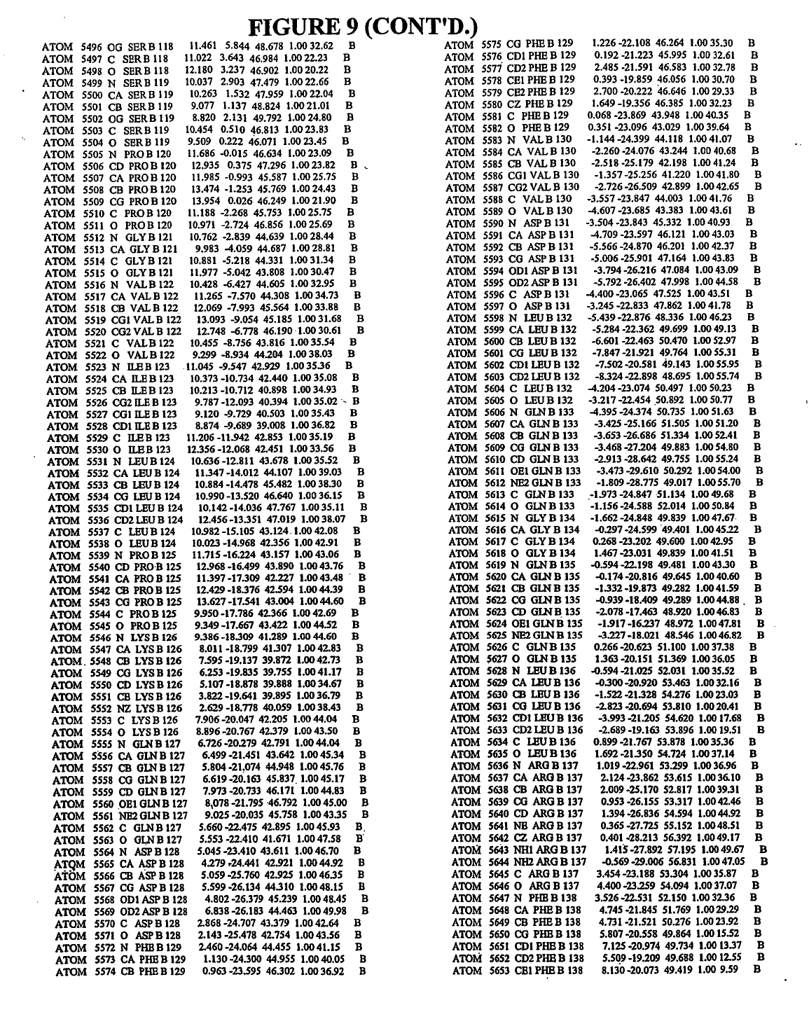

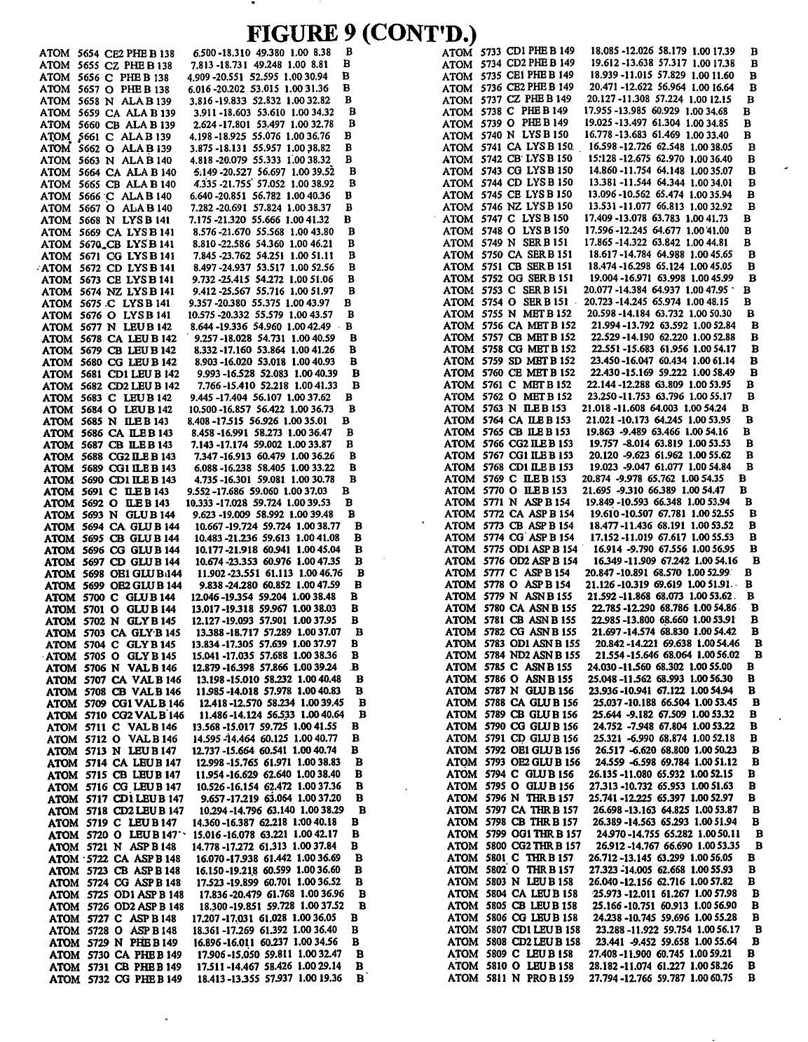

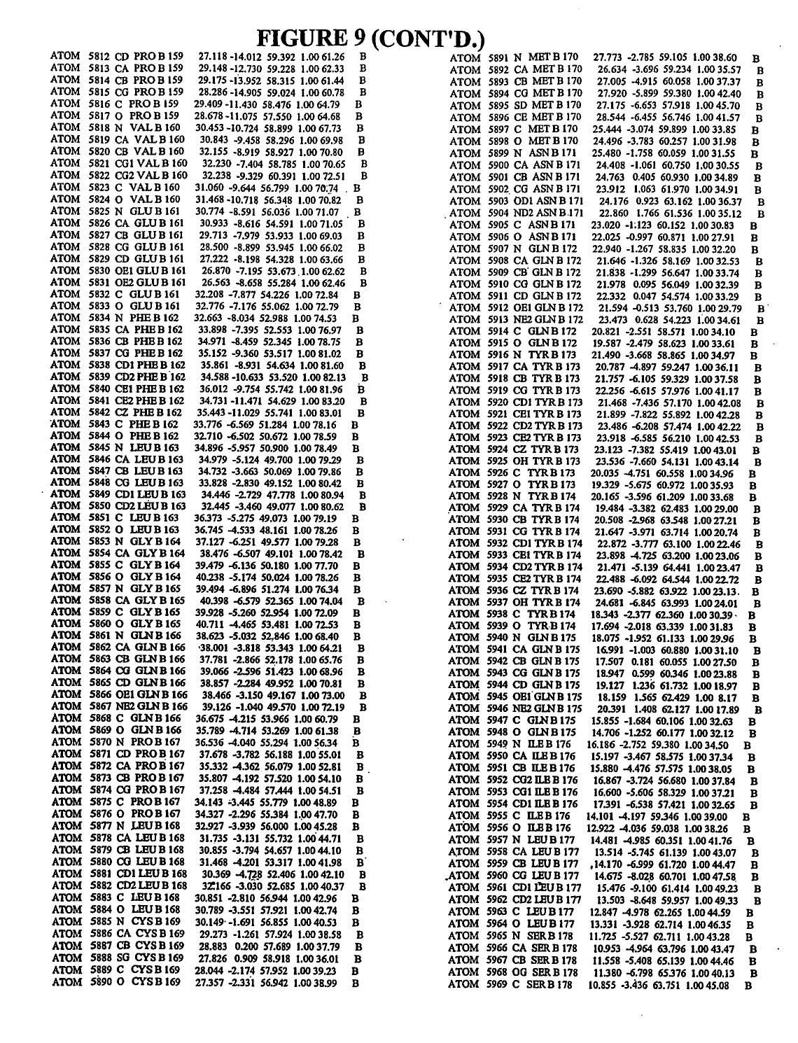

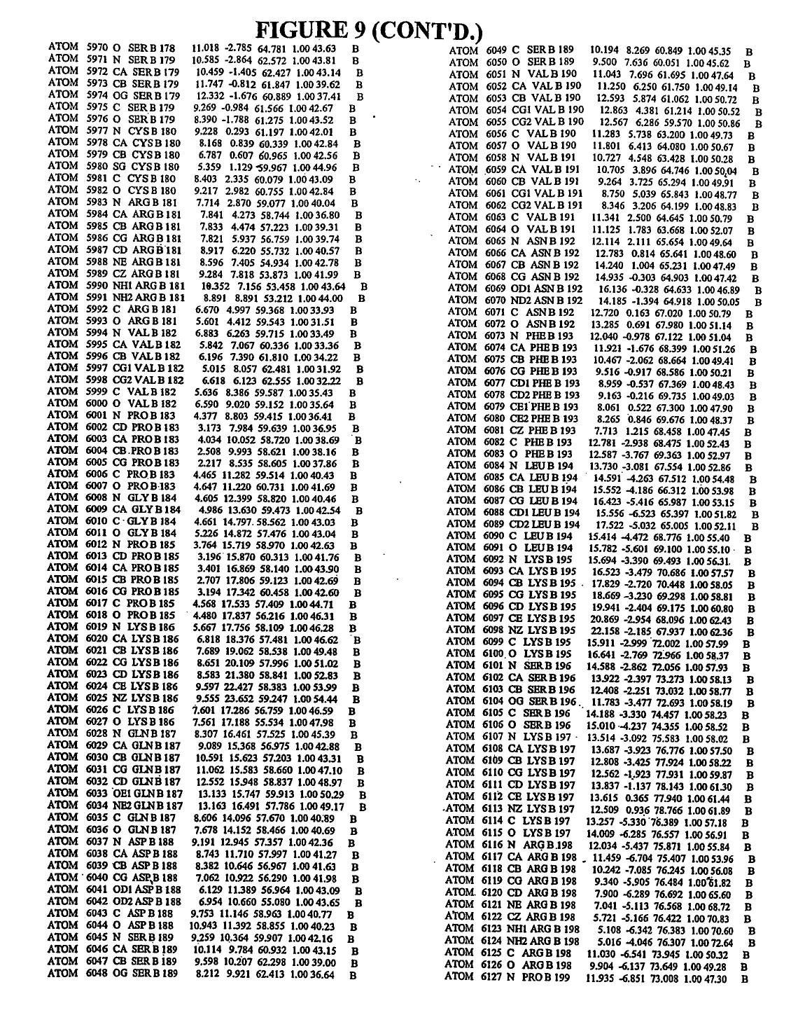

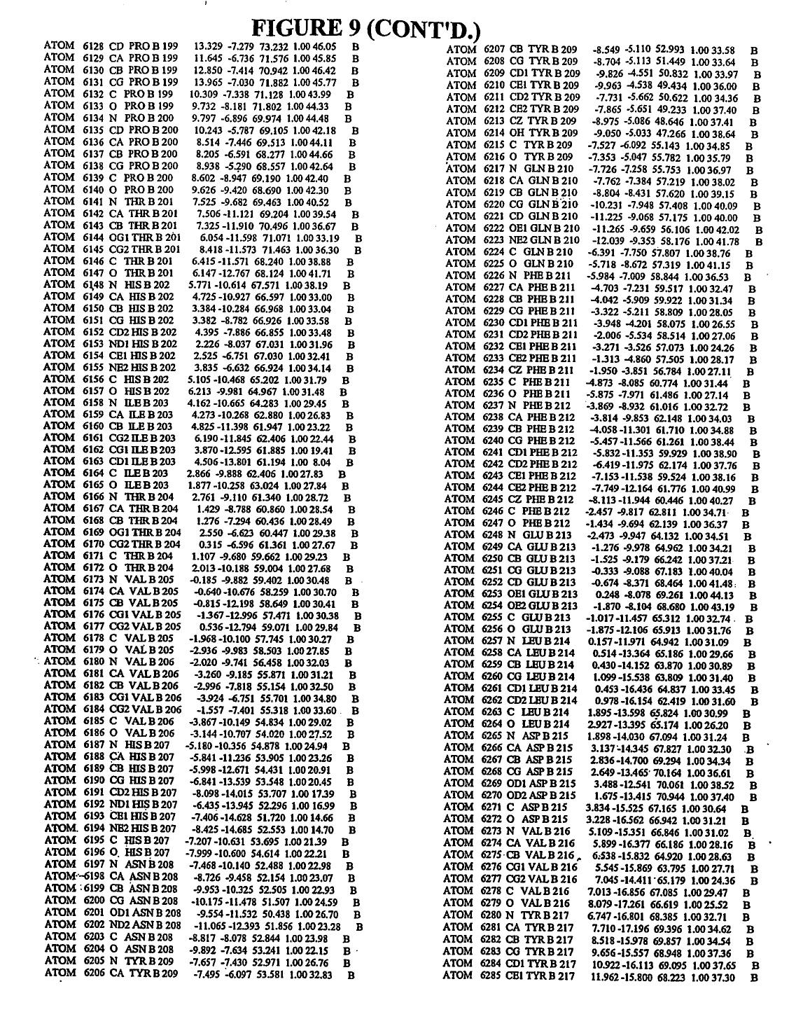

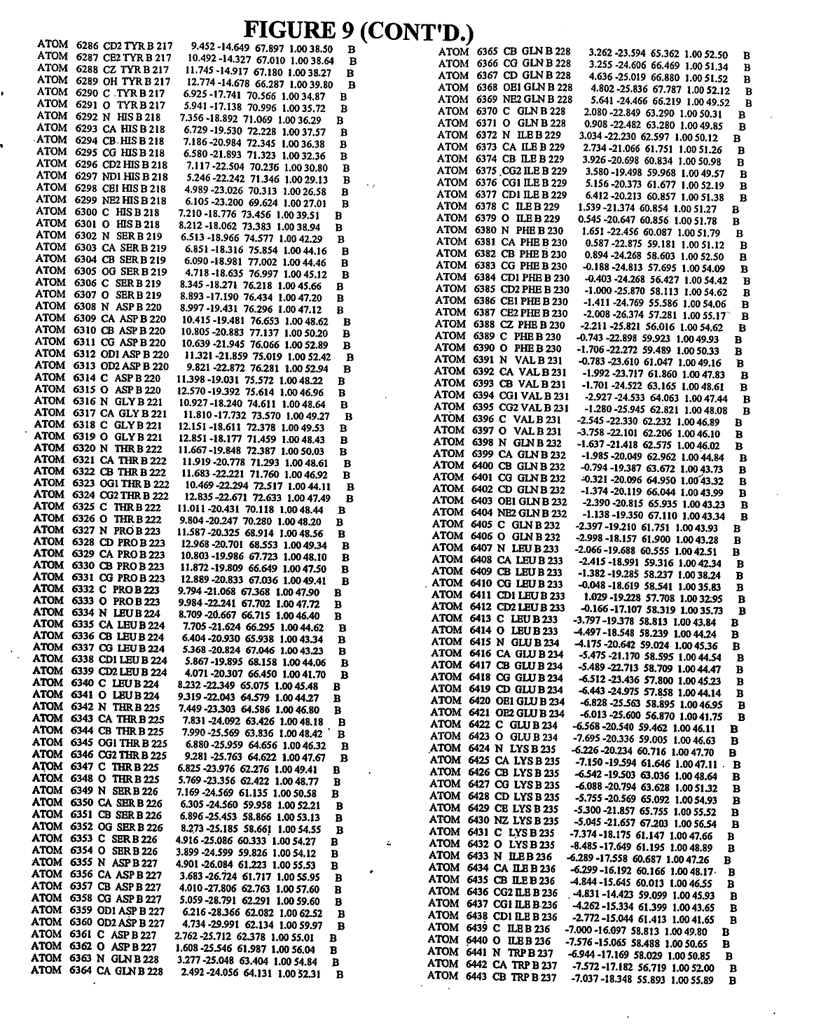

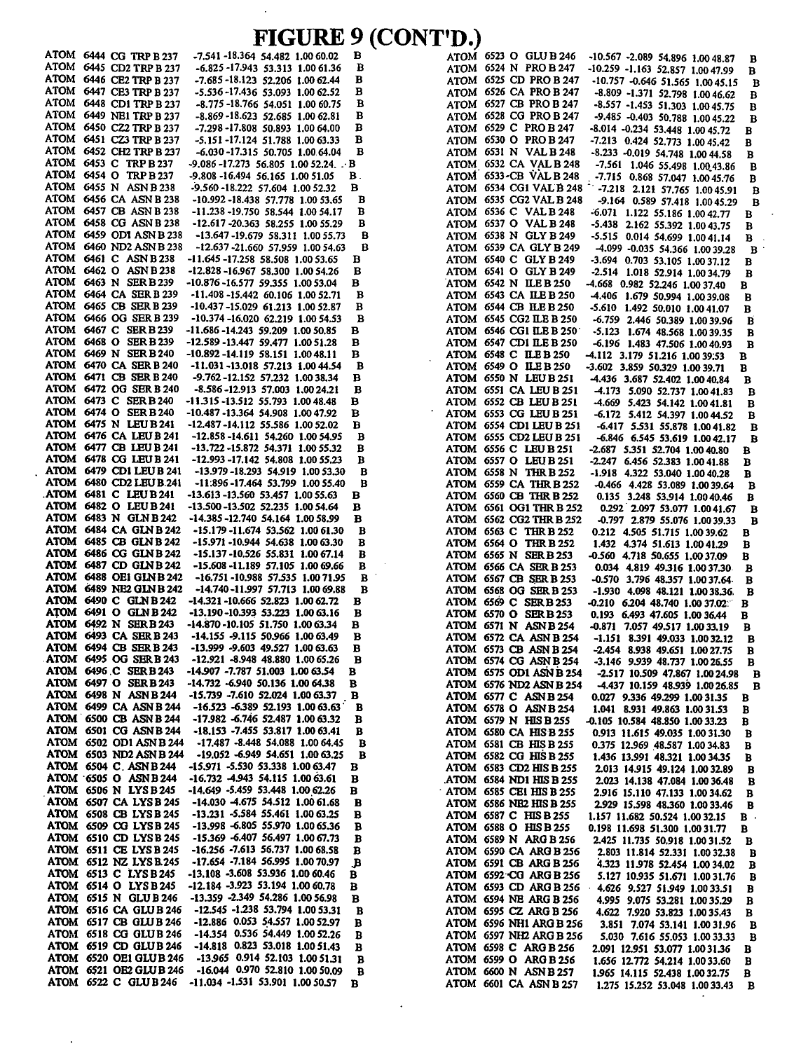

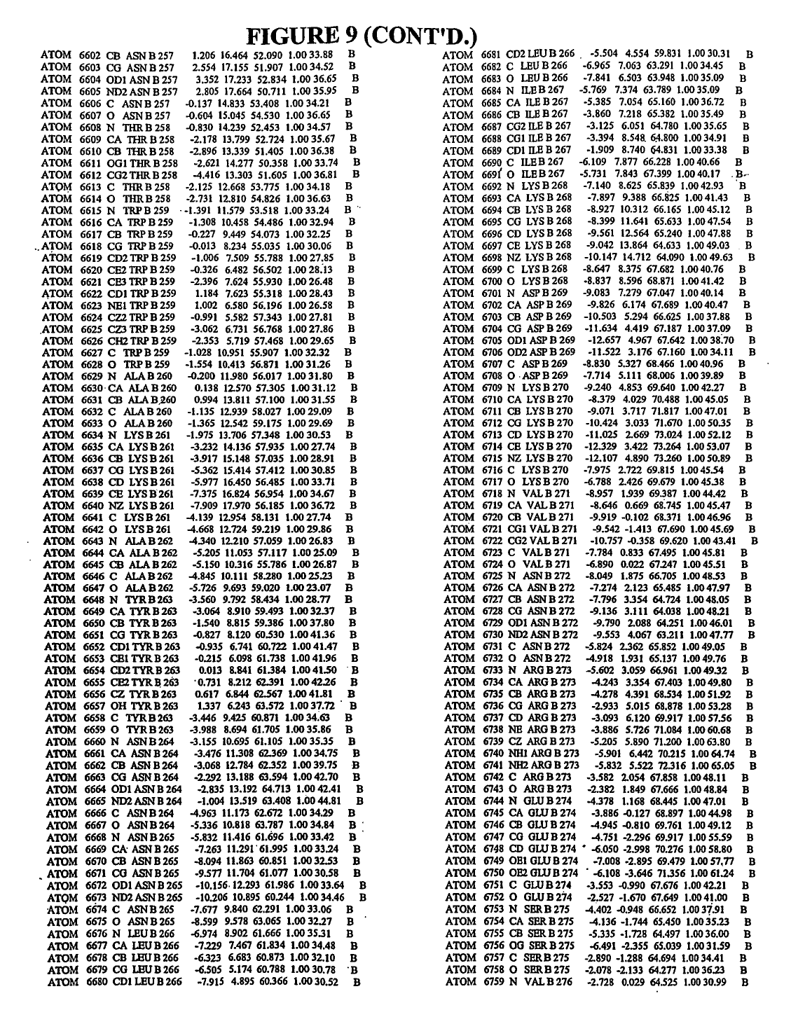

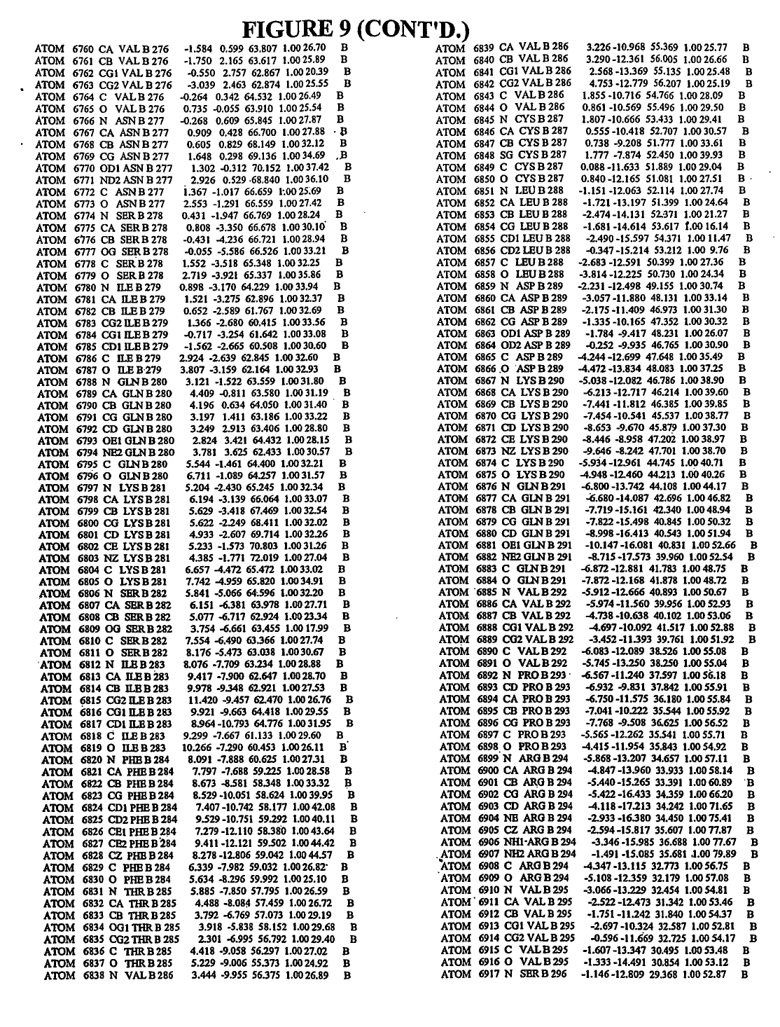

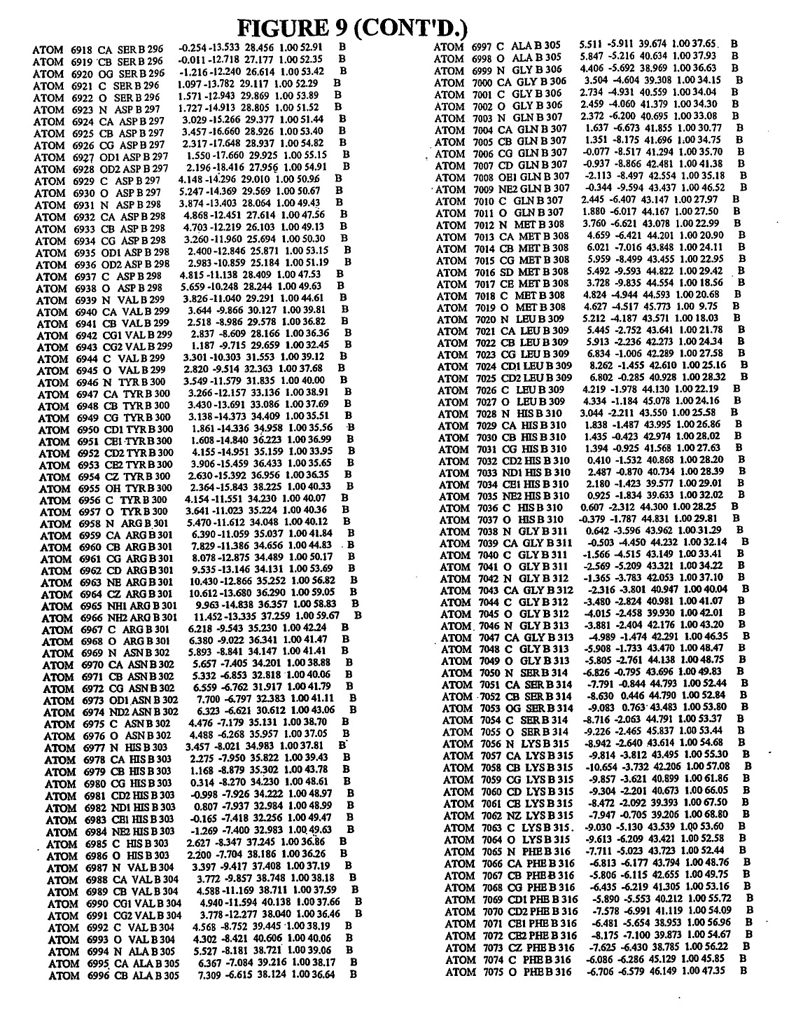

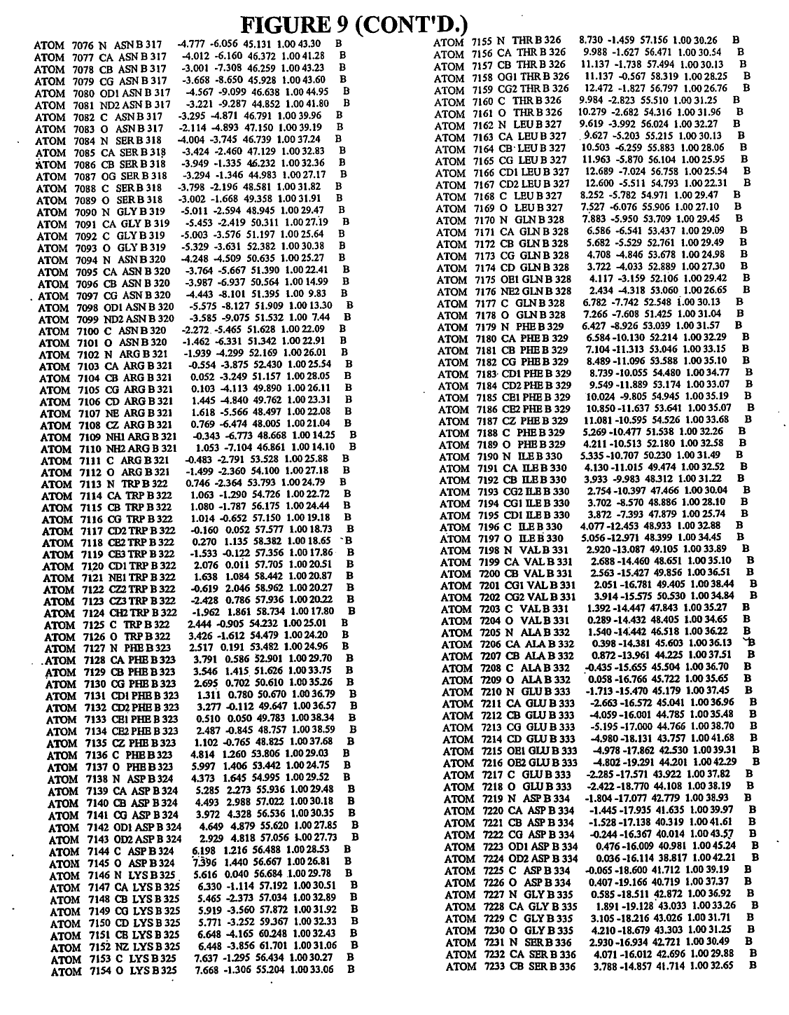

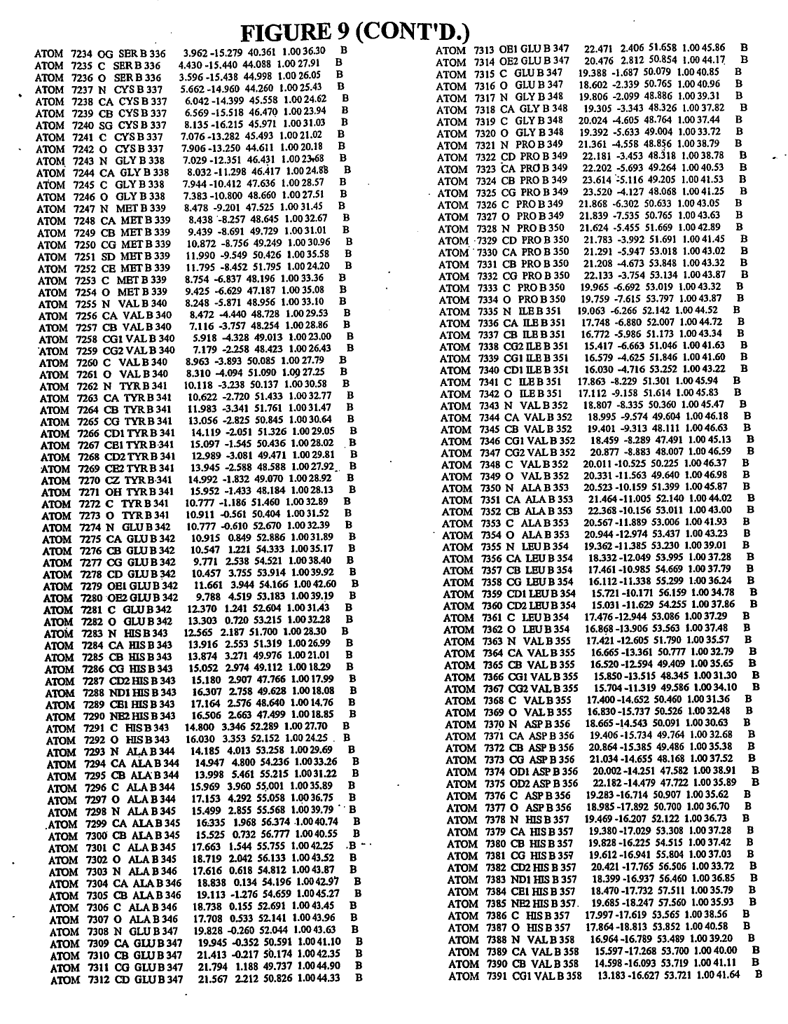

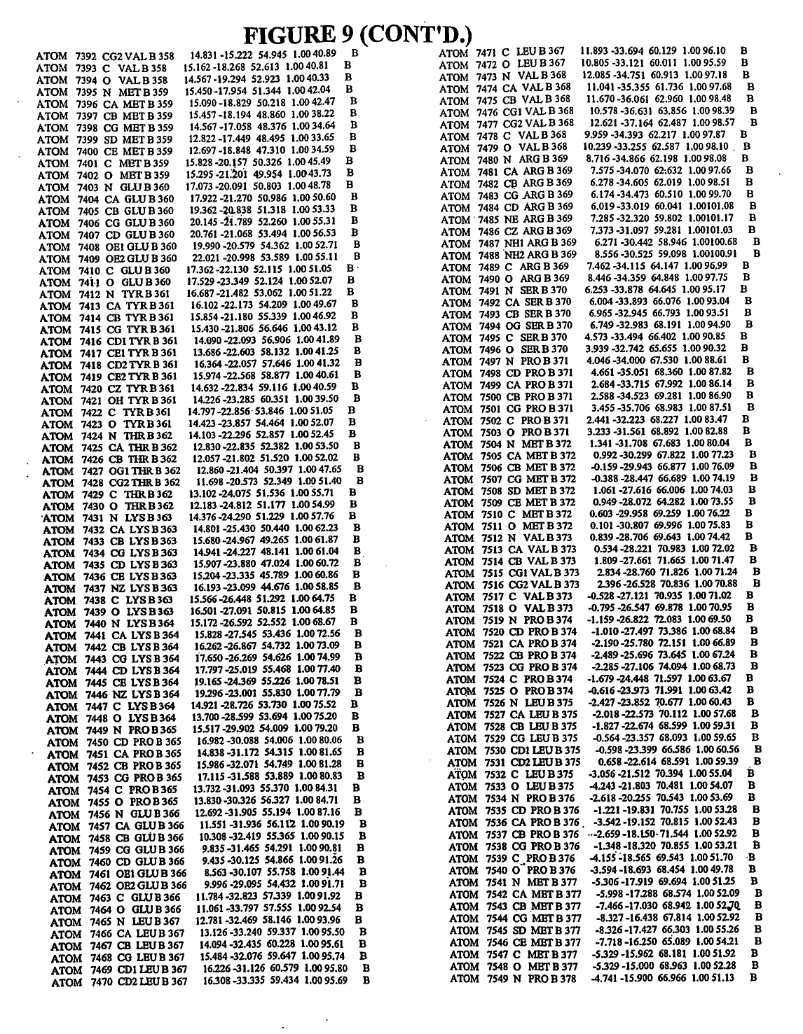

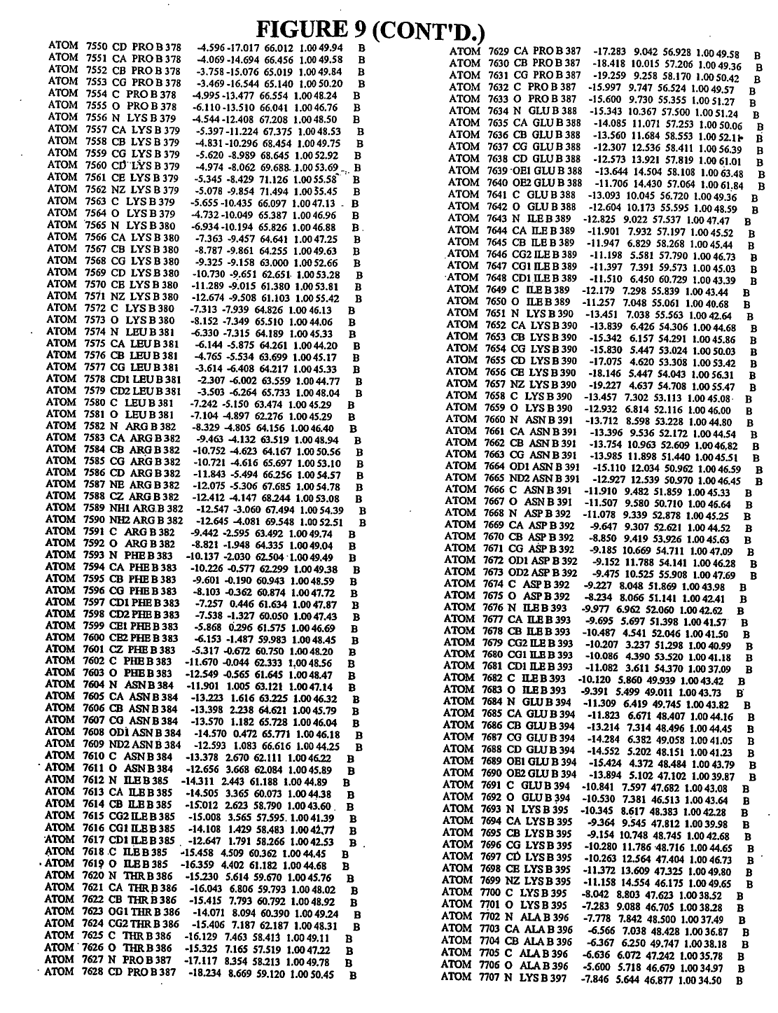

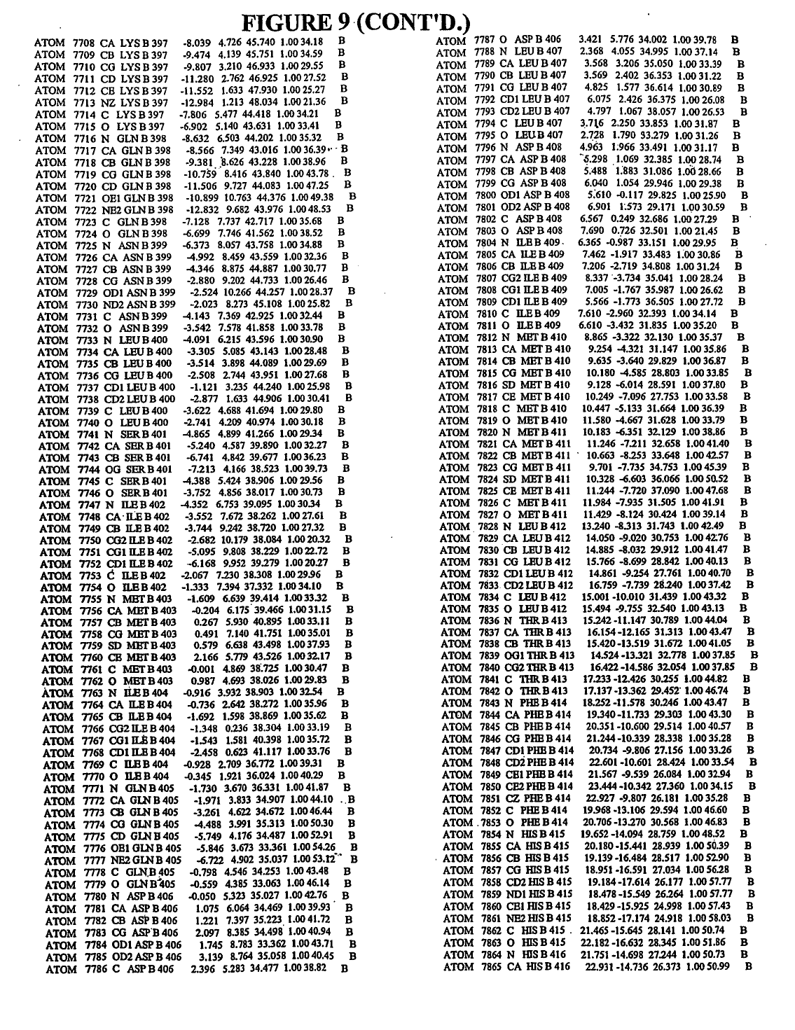

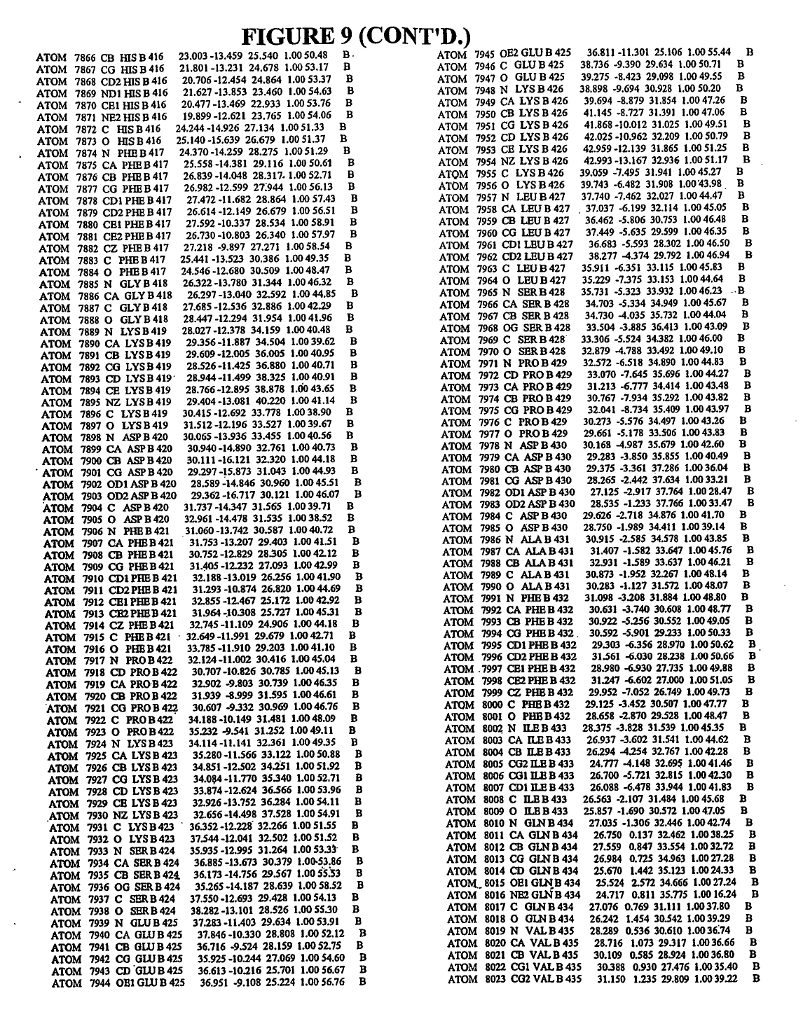

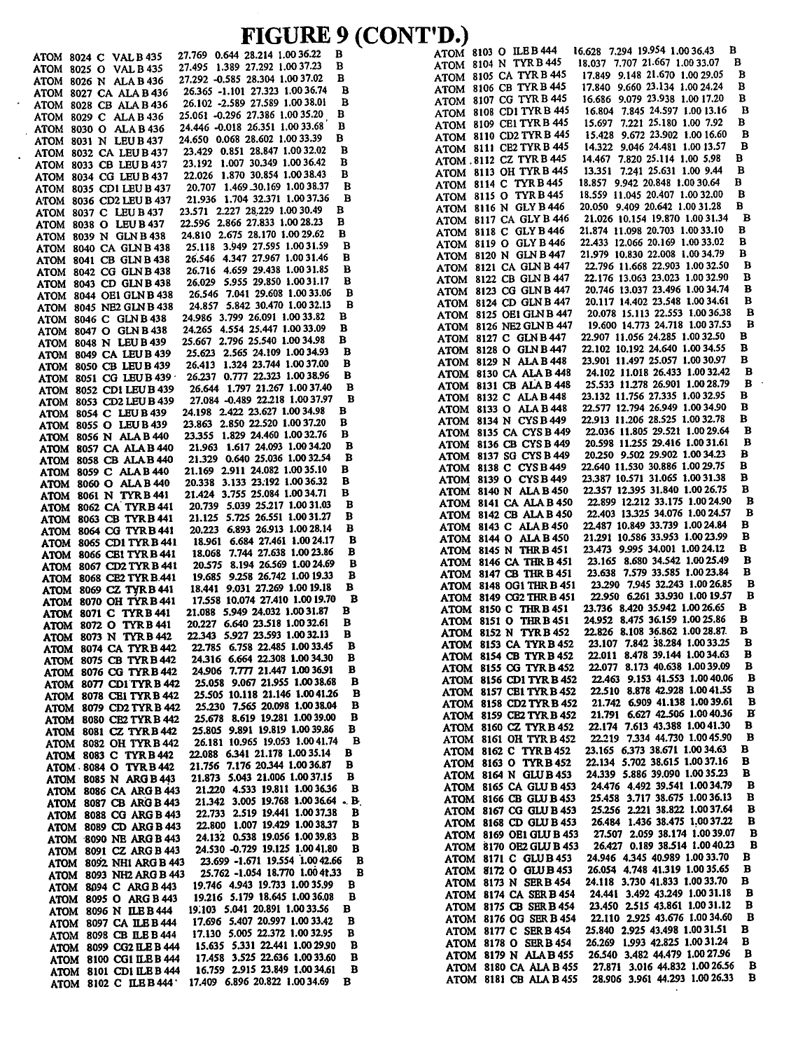

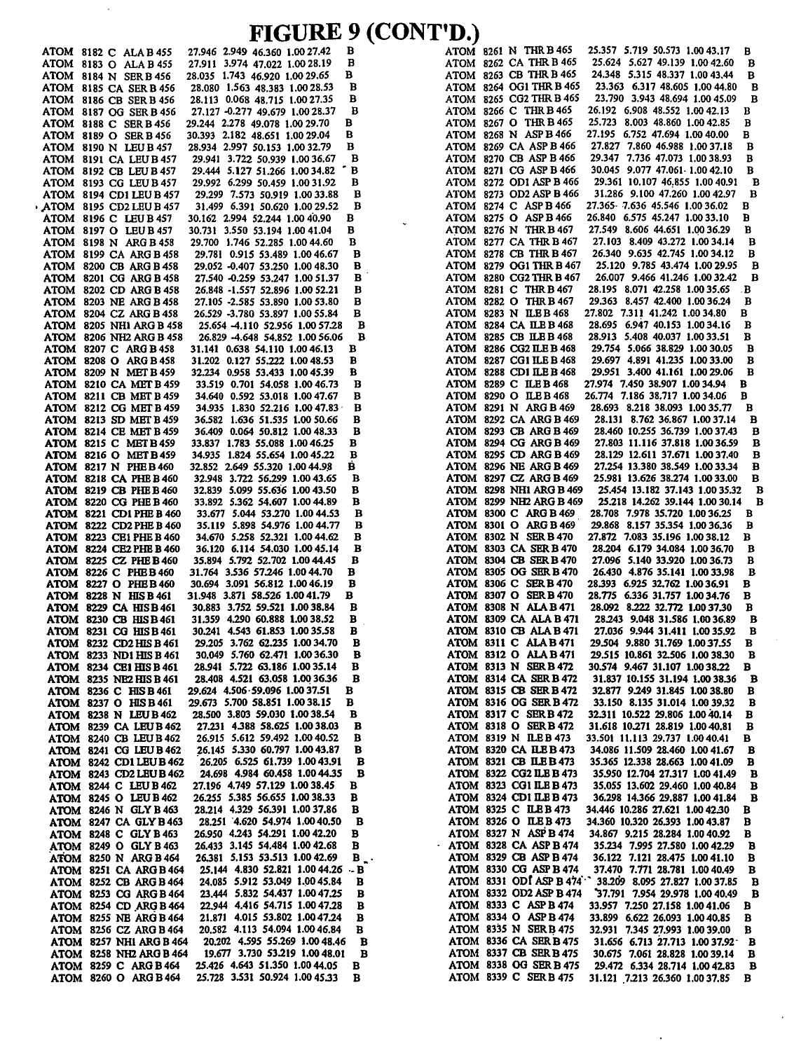

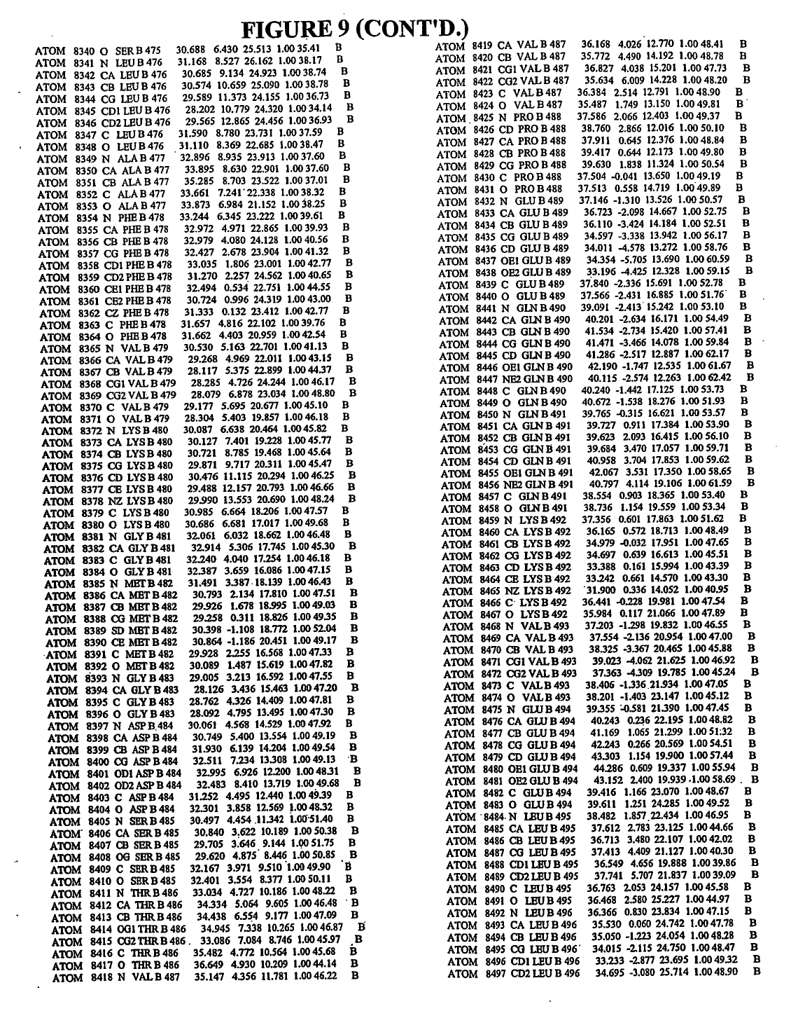

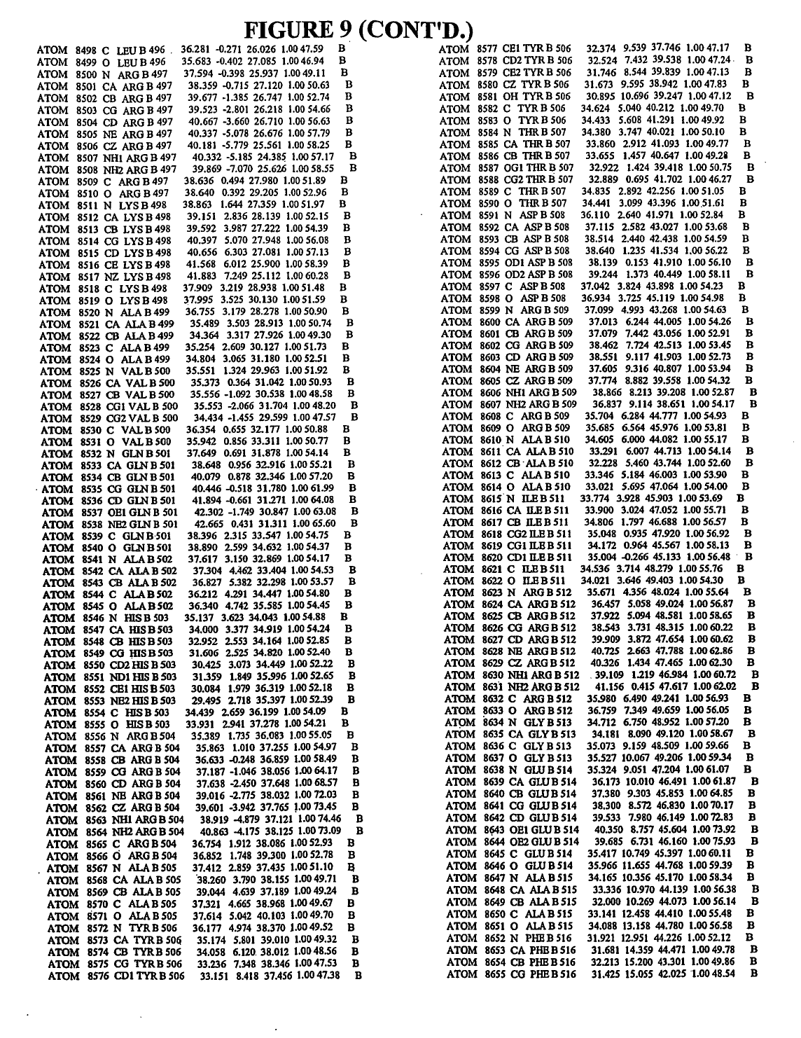

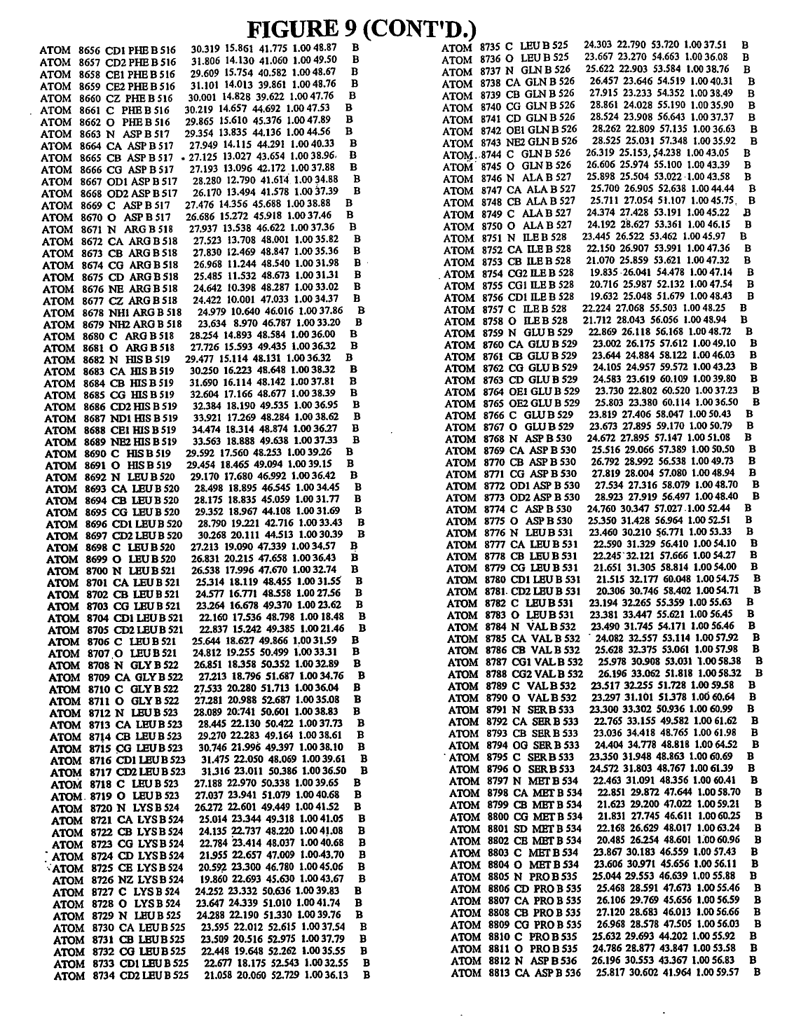

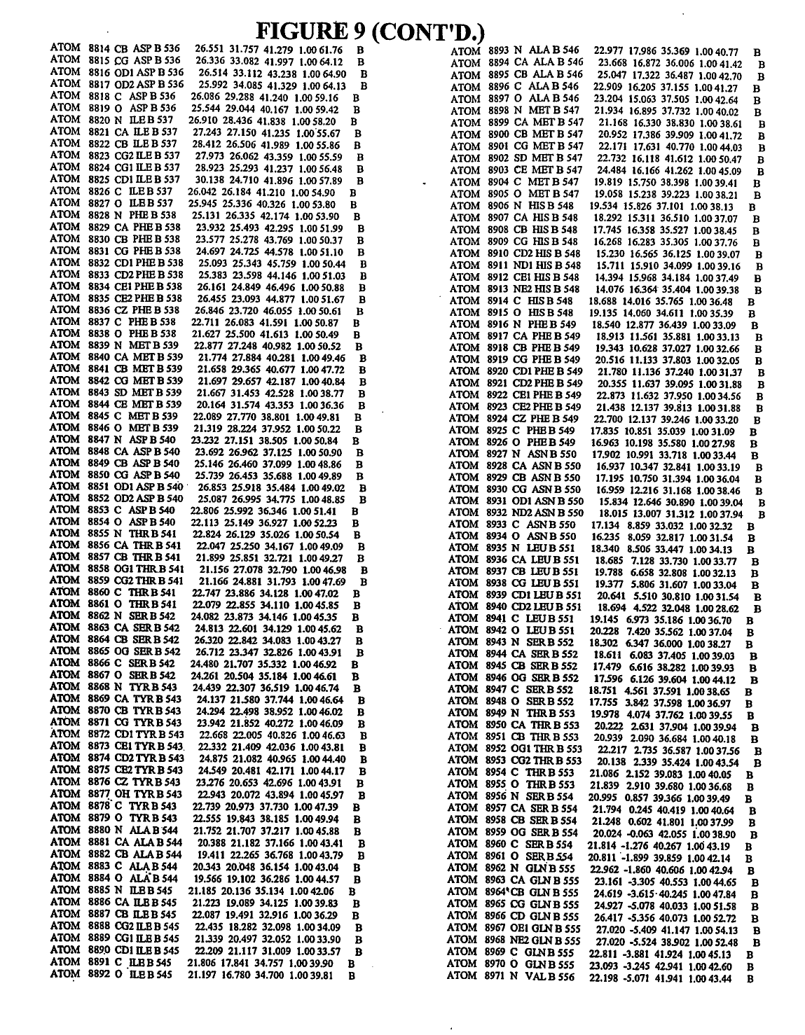

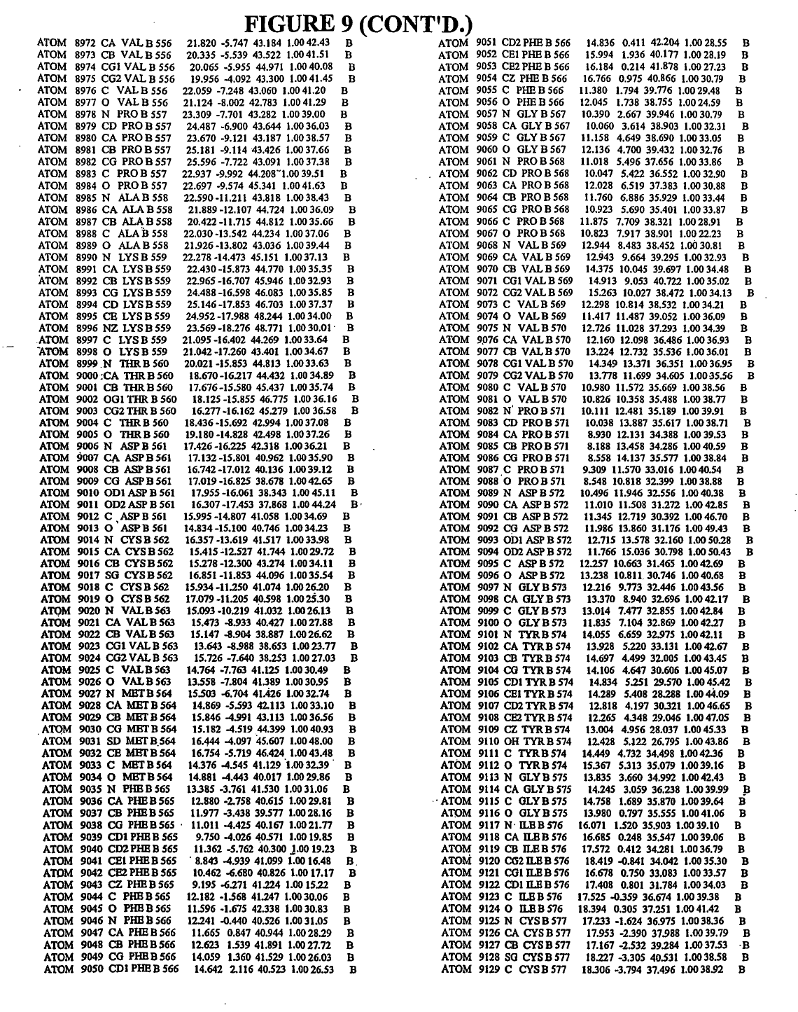

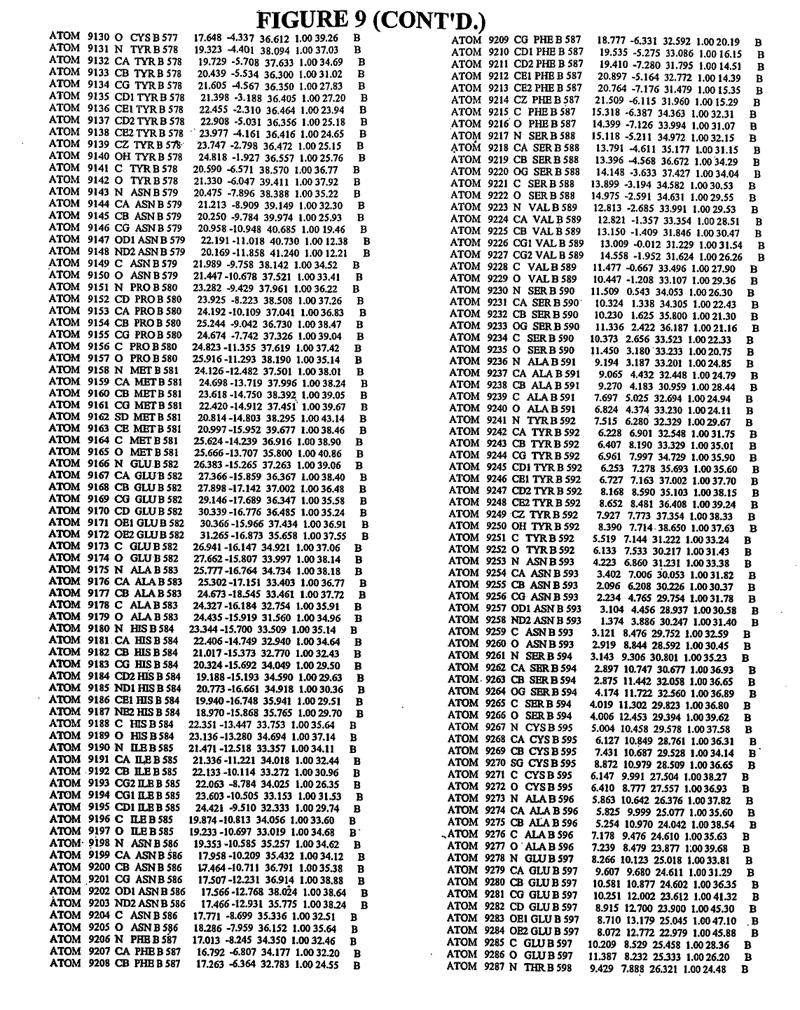

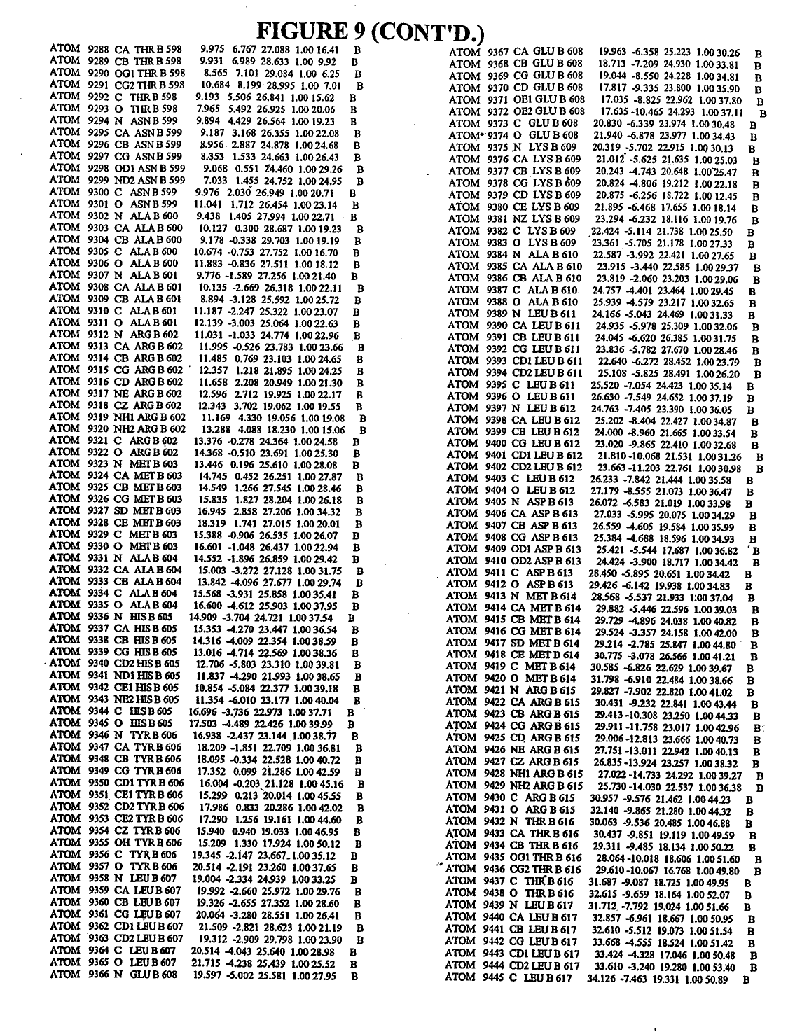

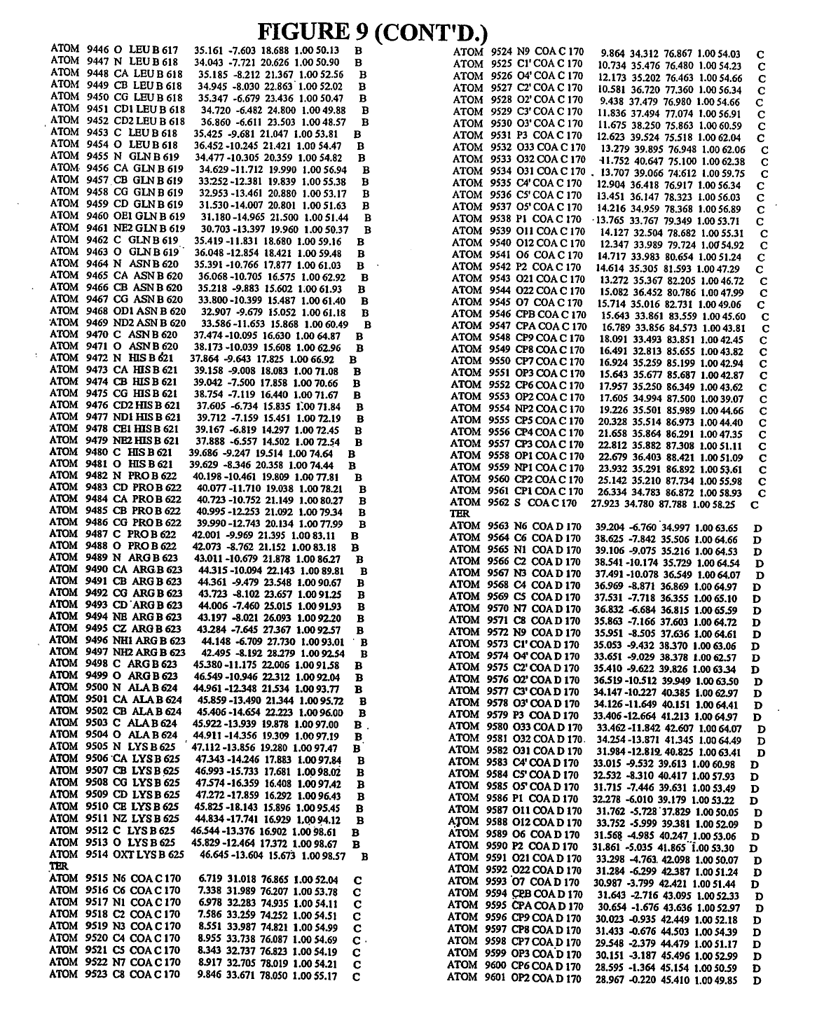

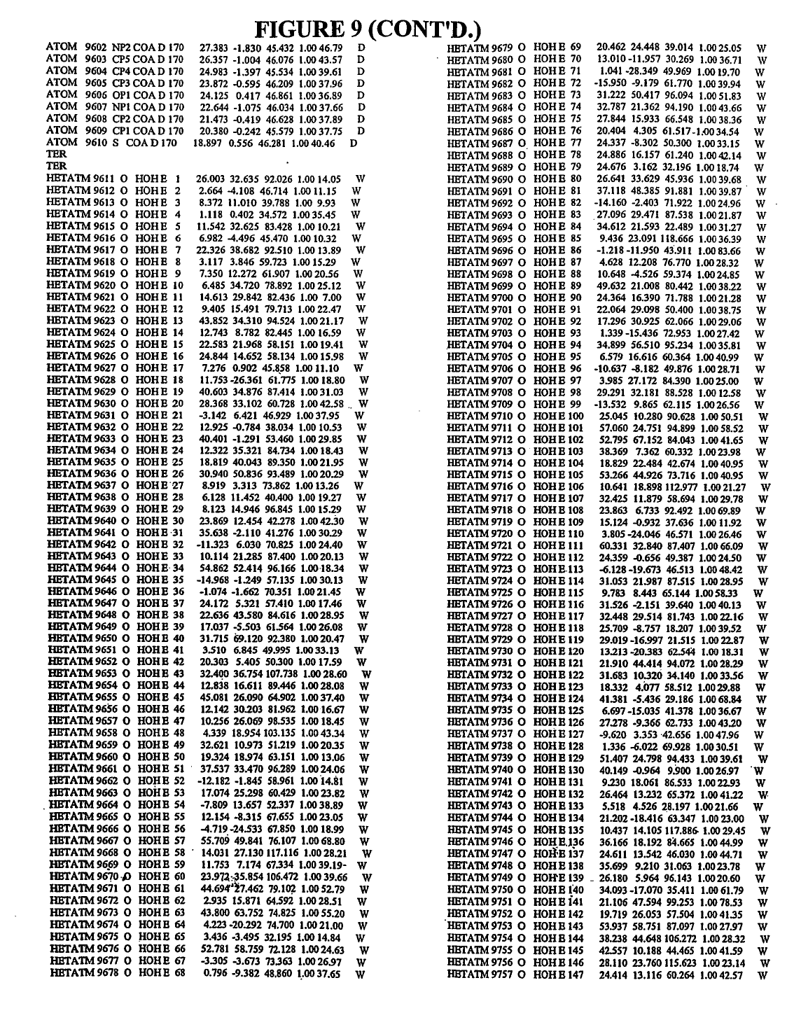

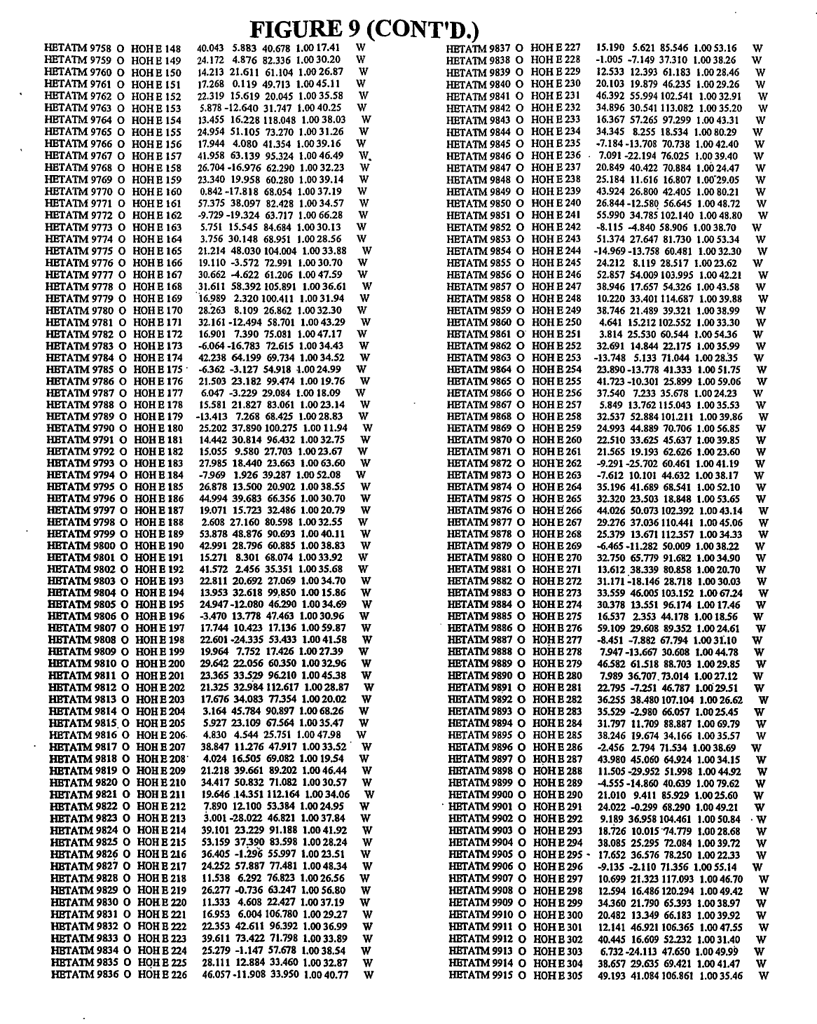

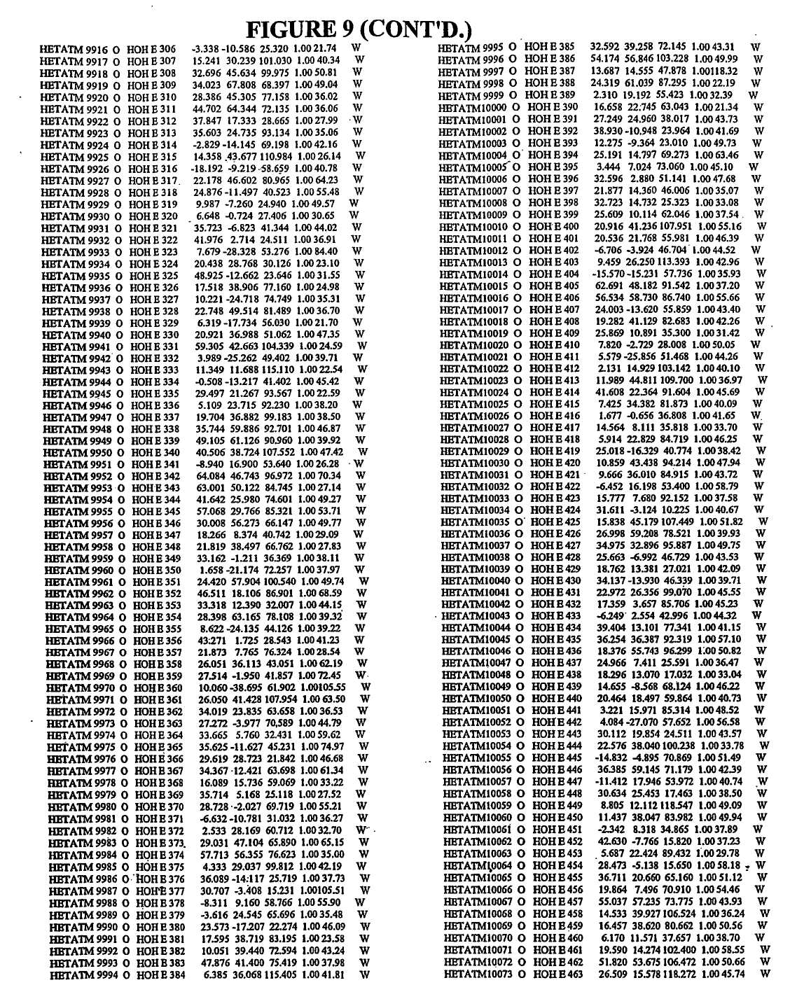

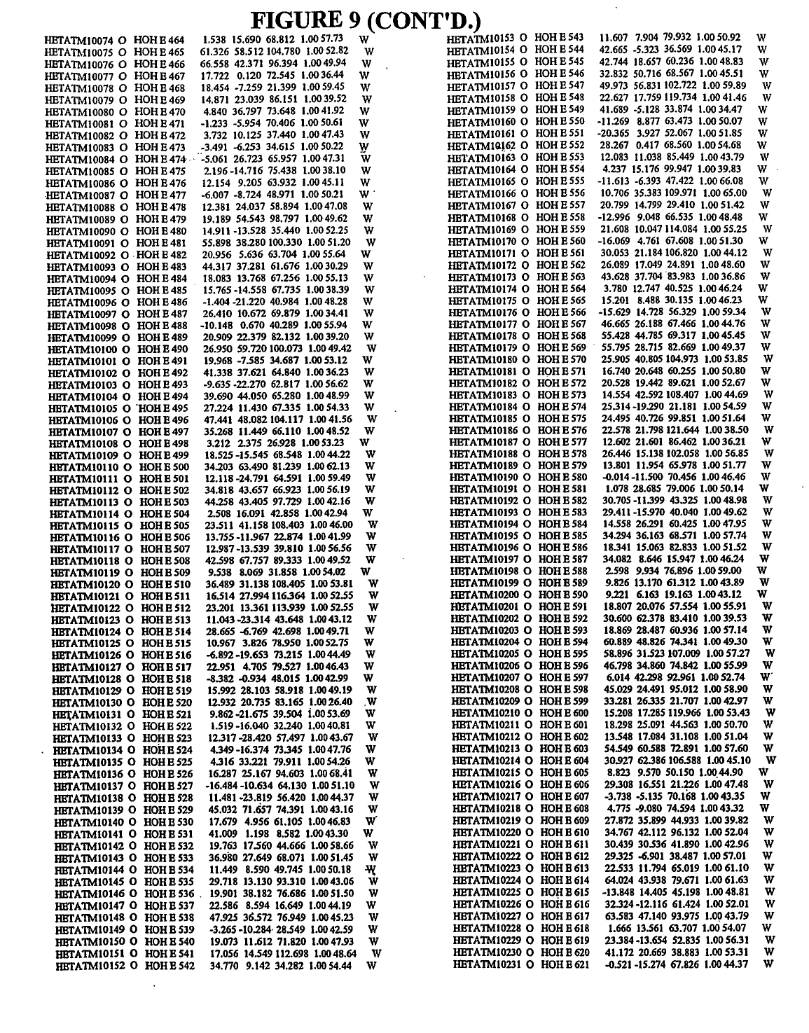

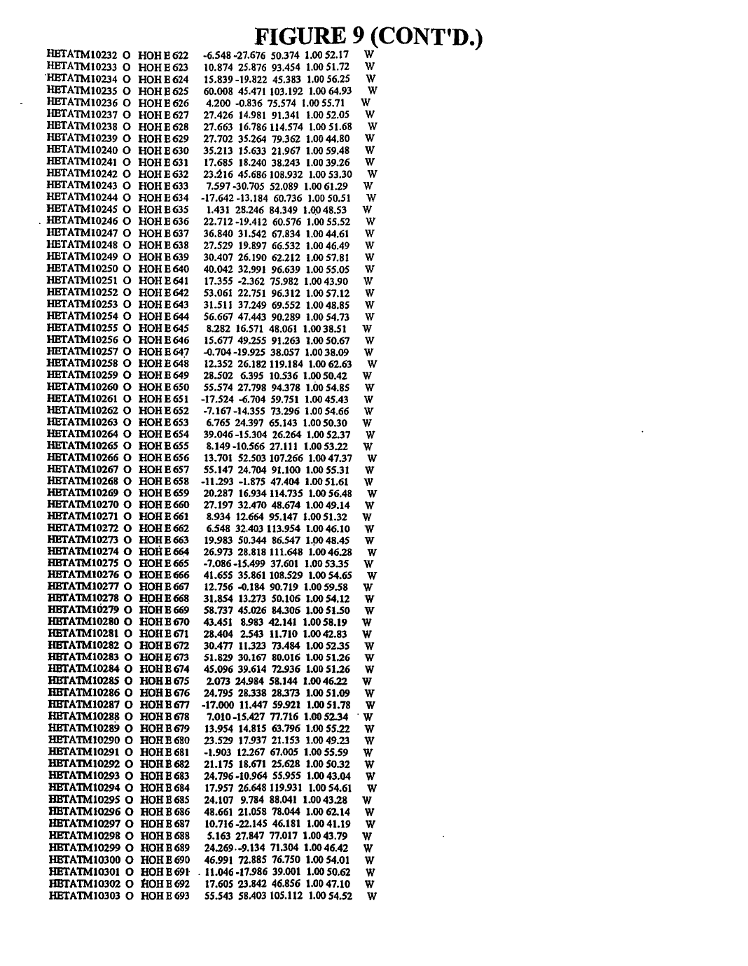

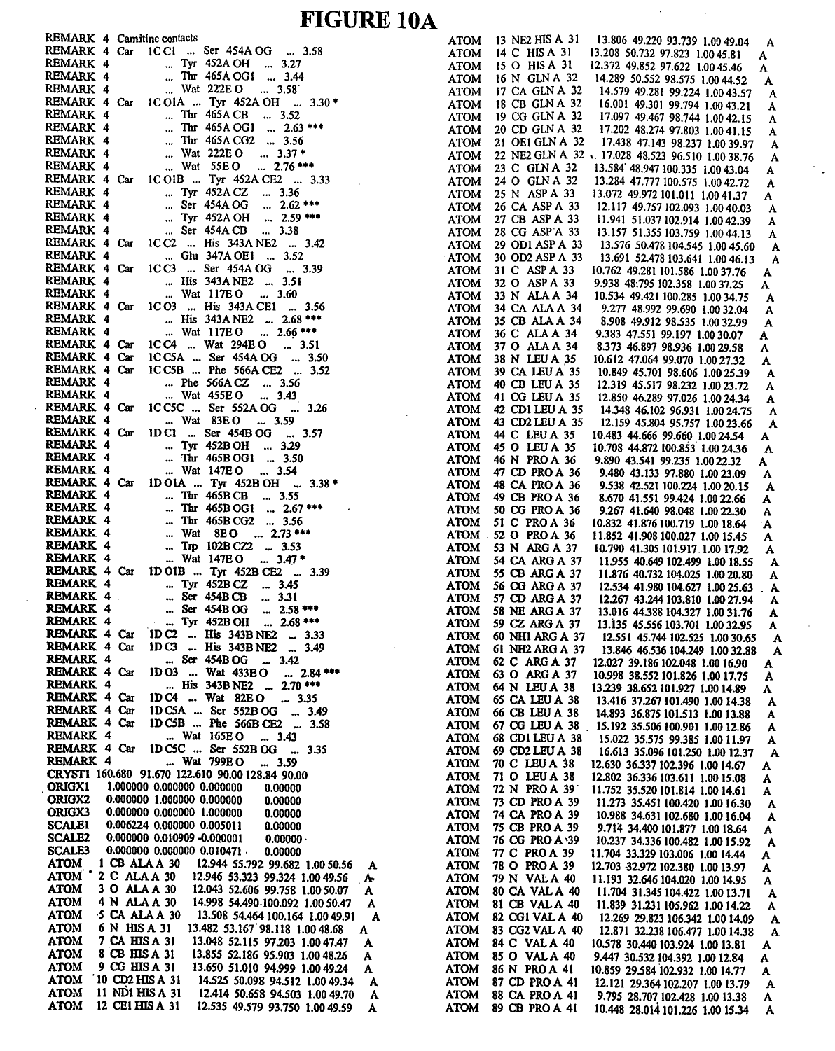

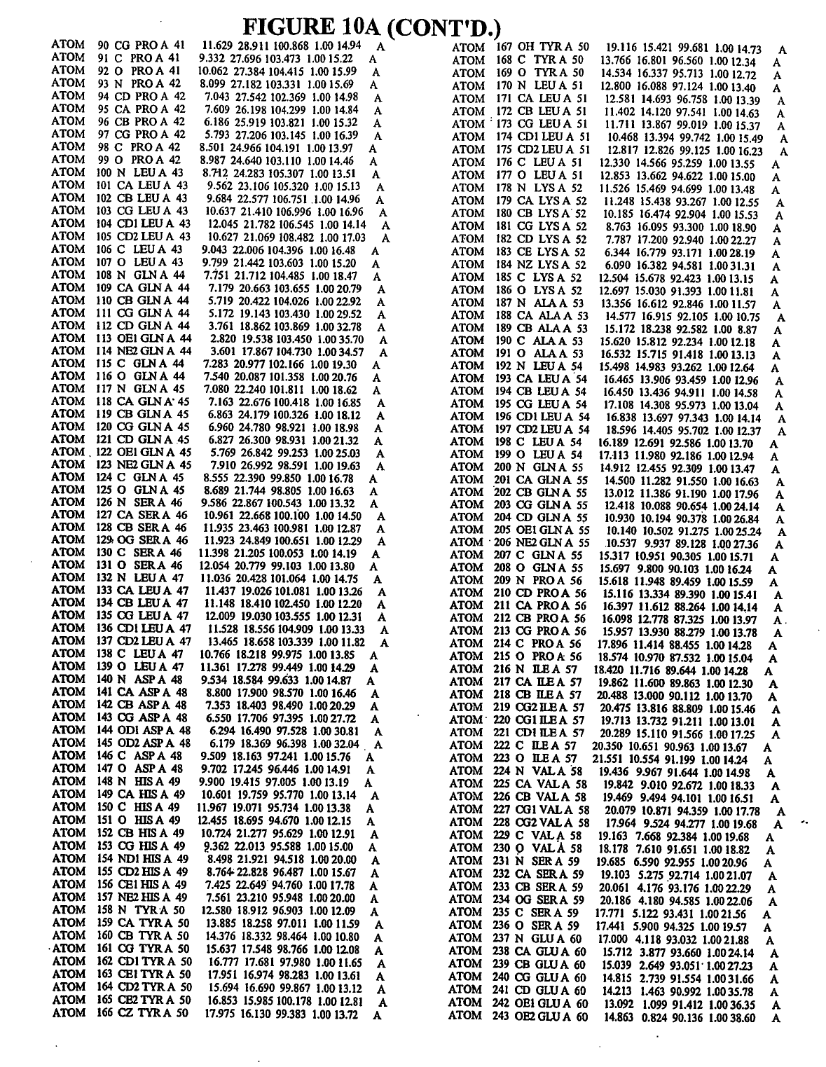

FIG. 9 lists the atomic coordinates of the mCRAT:CoA complex of the present invention; and

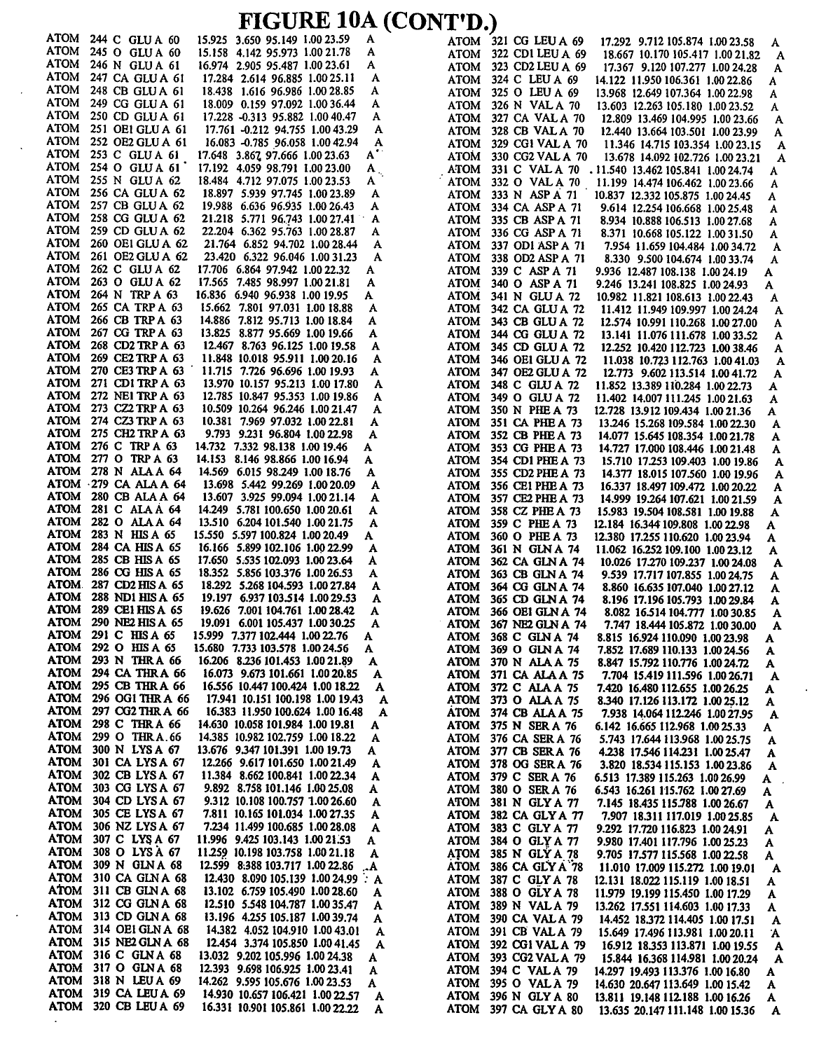

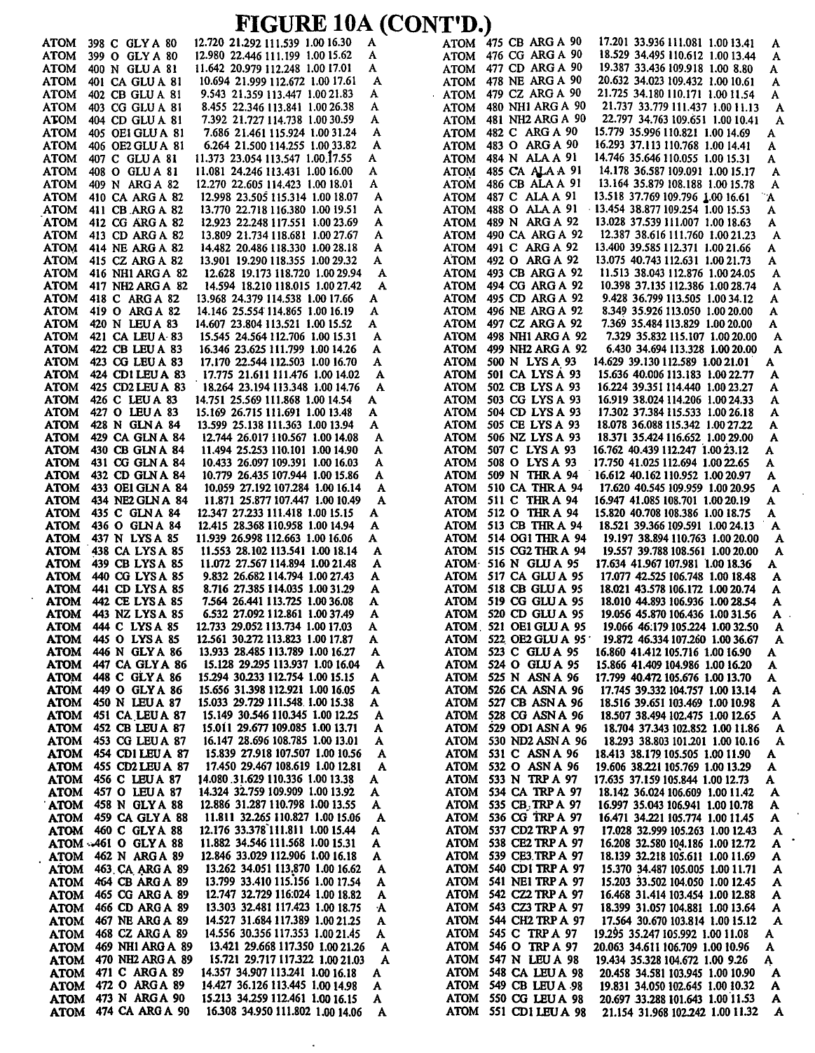

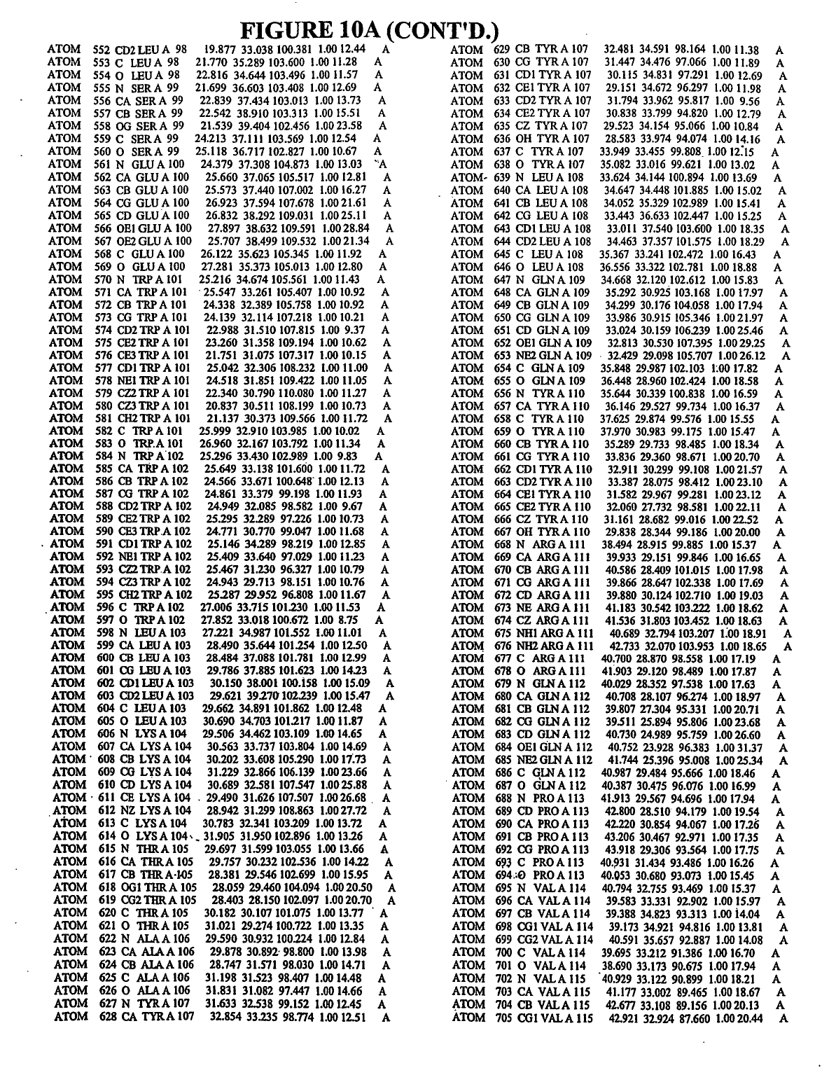

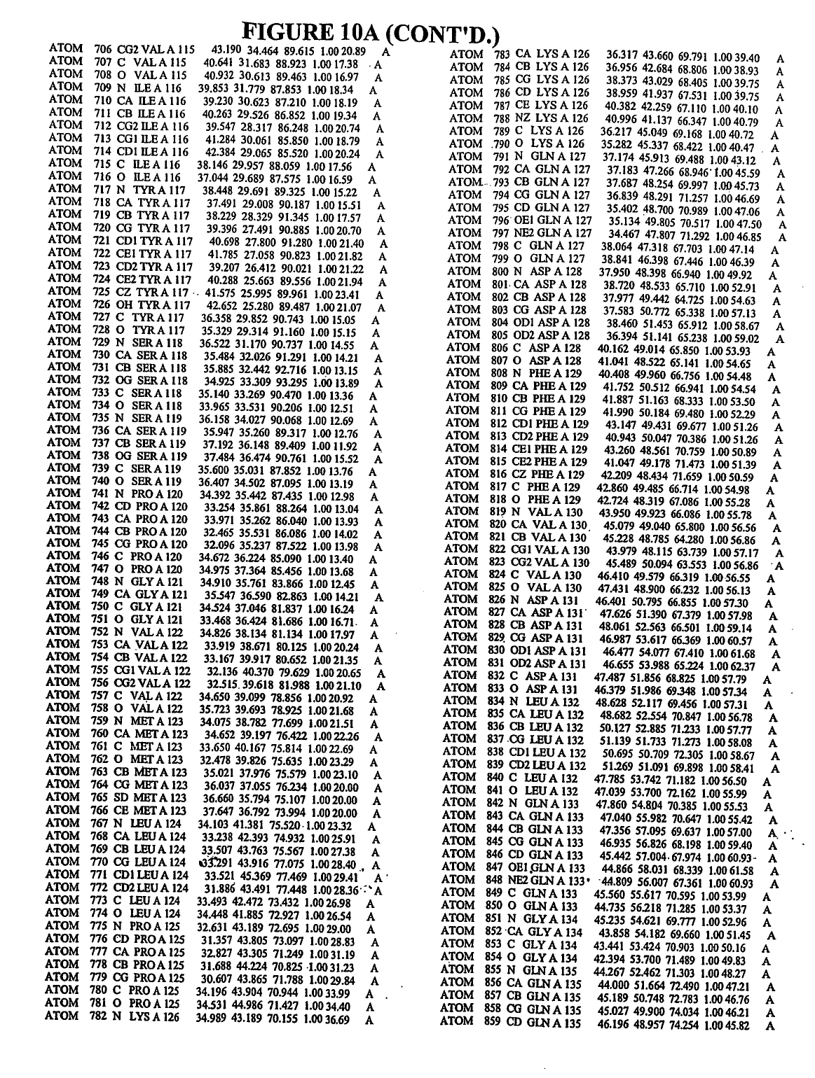

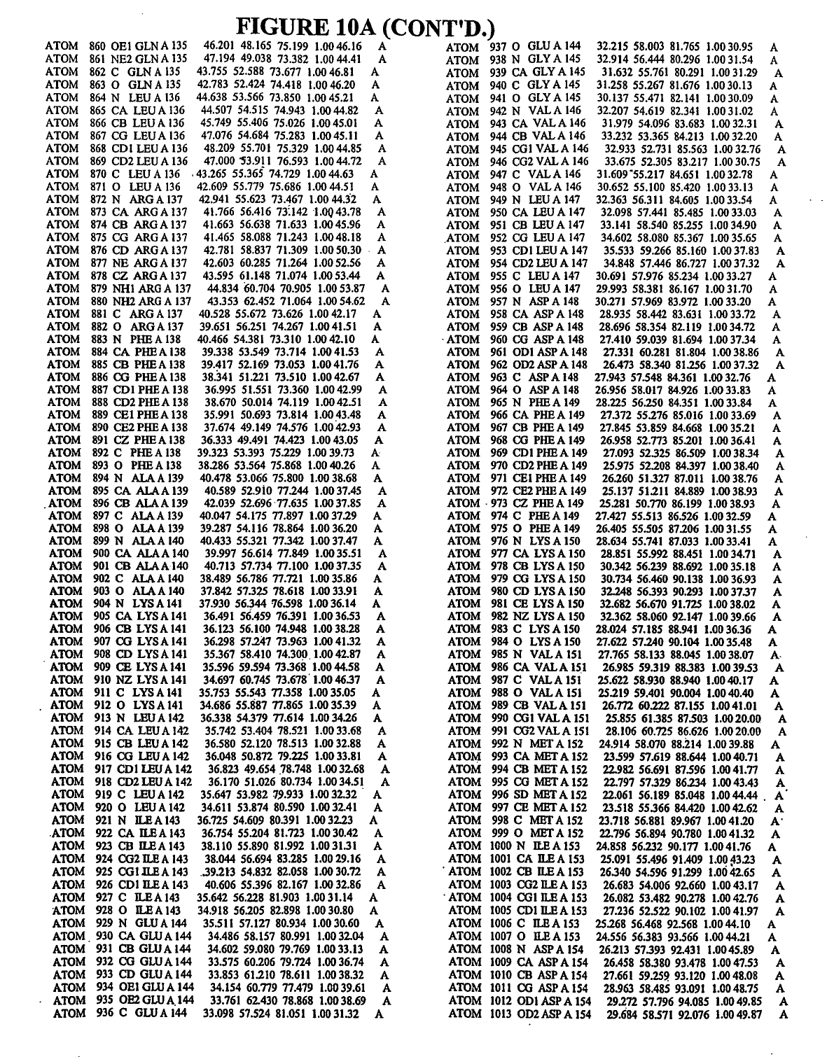

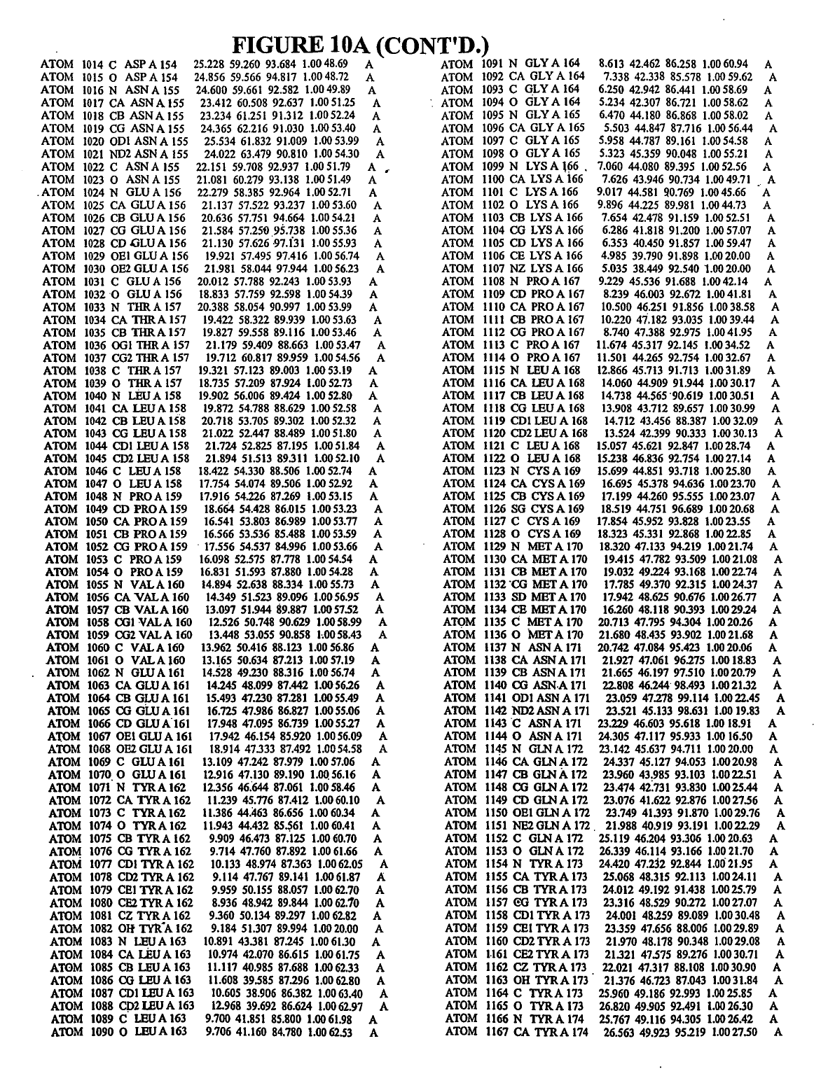

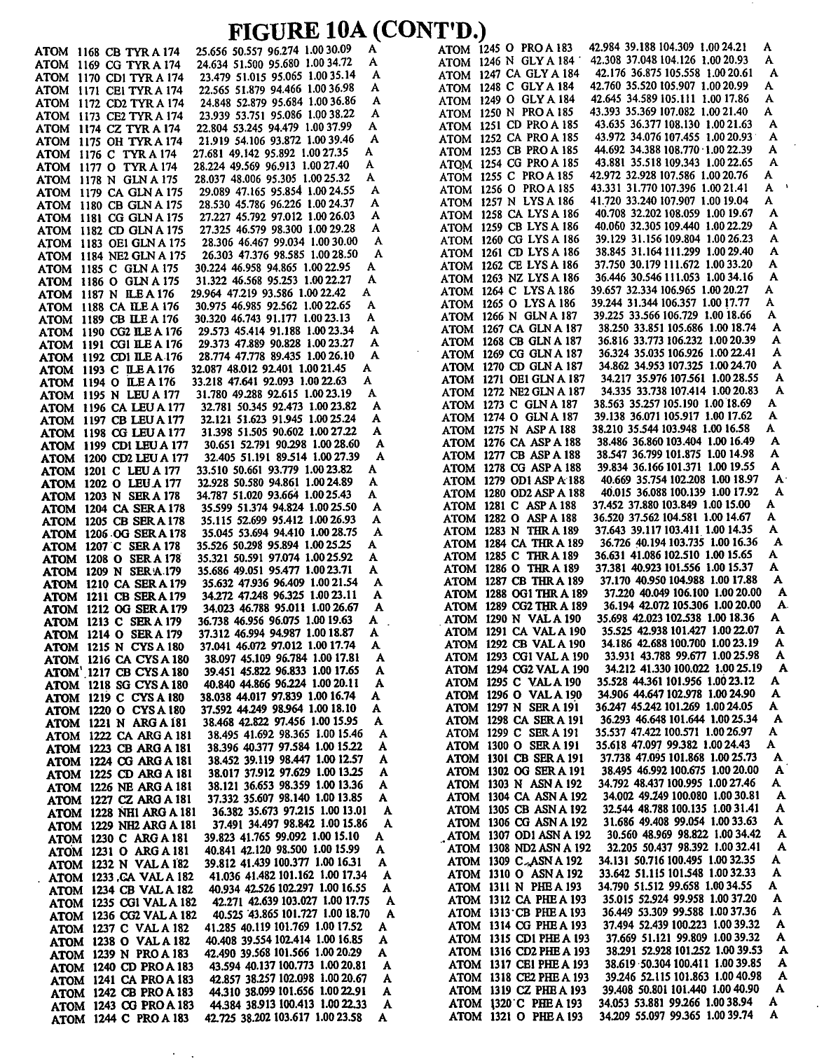

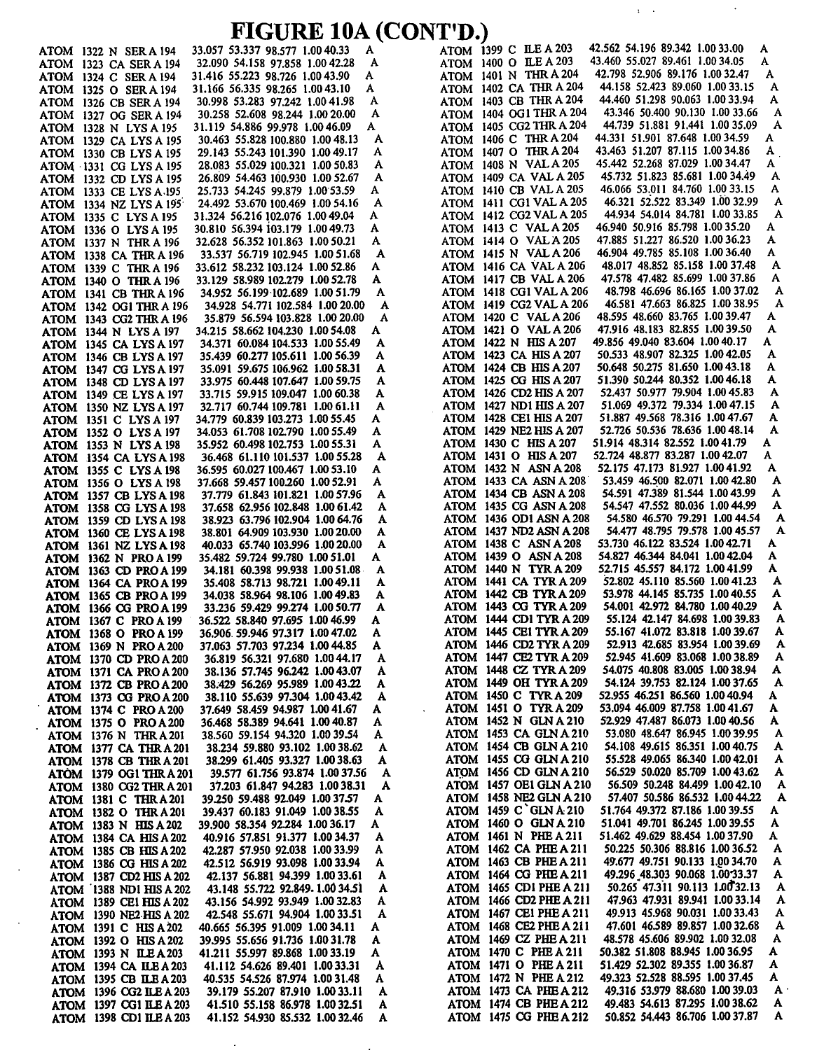

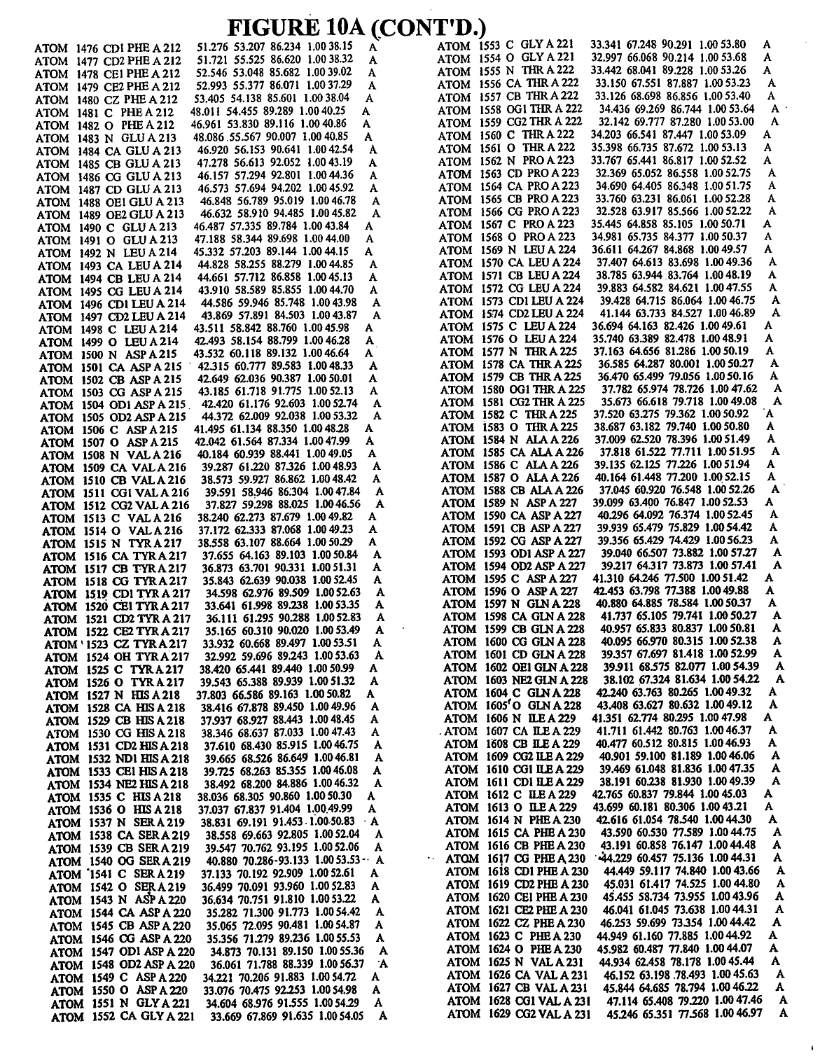

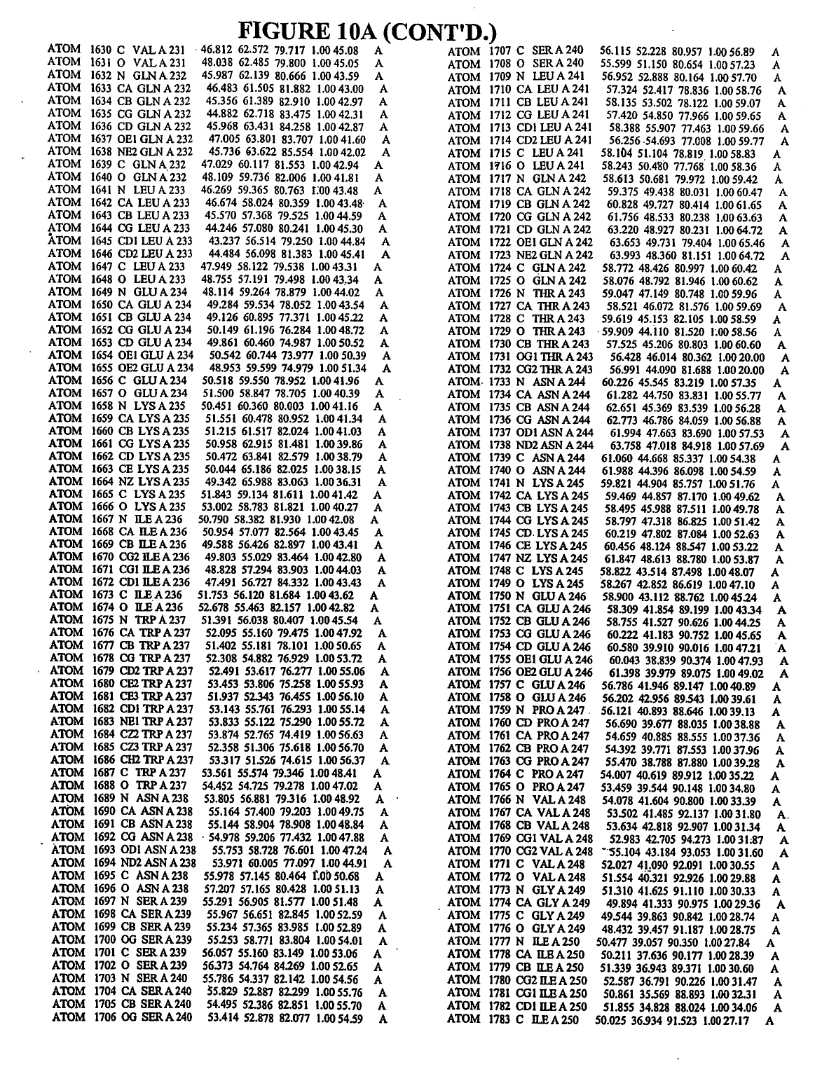

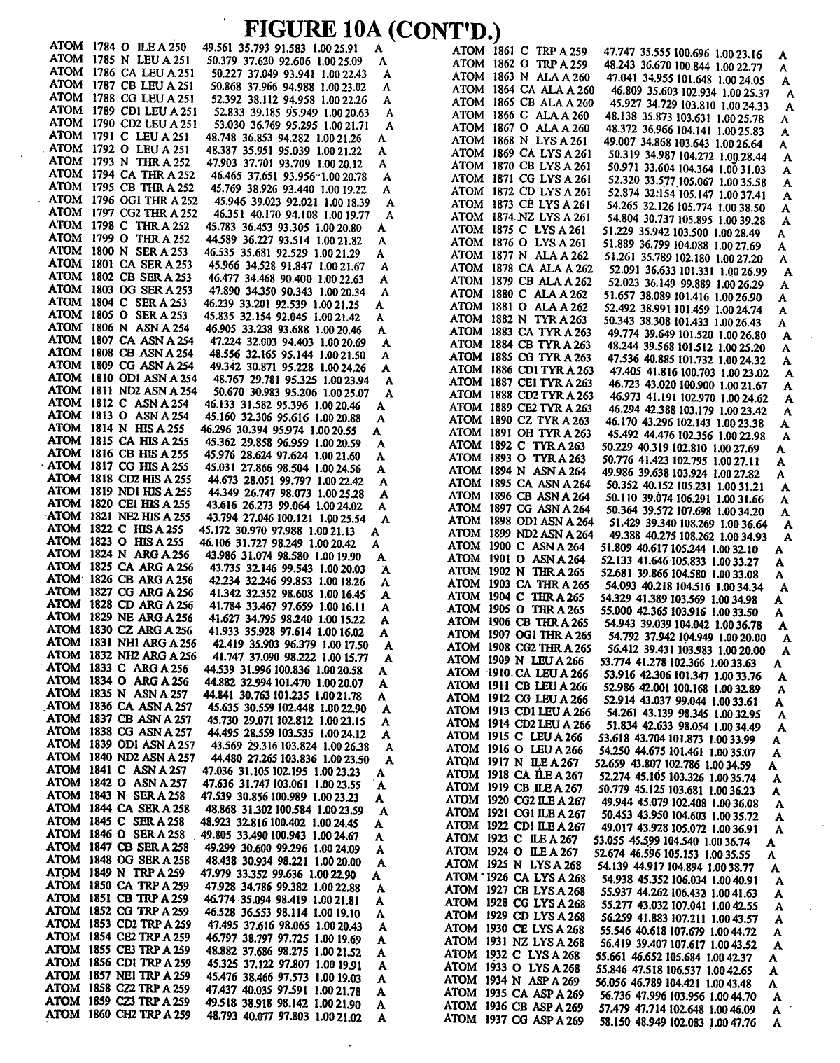

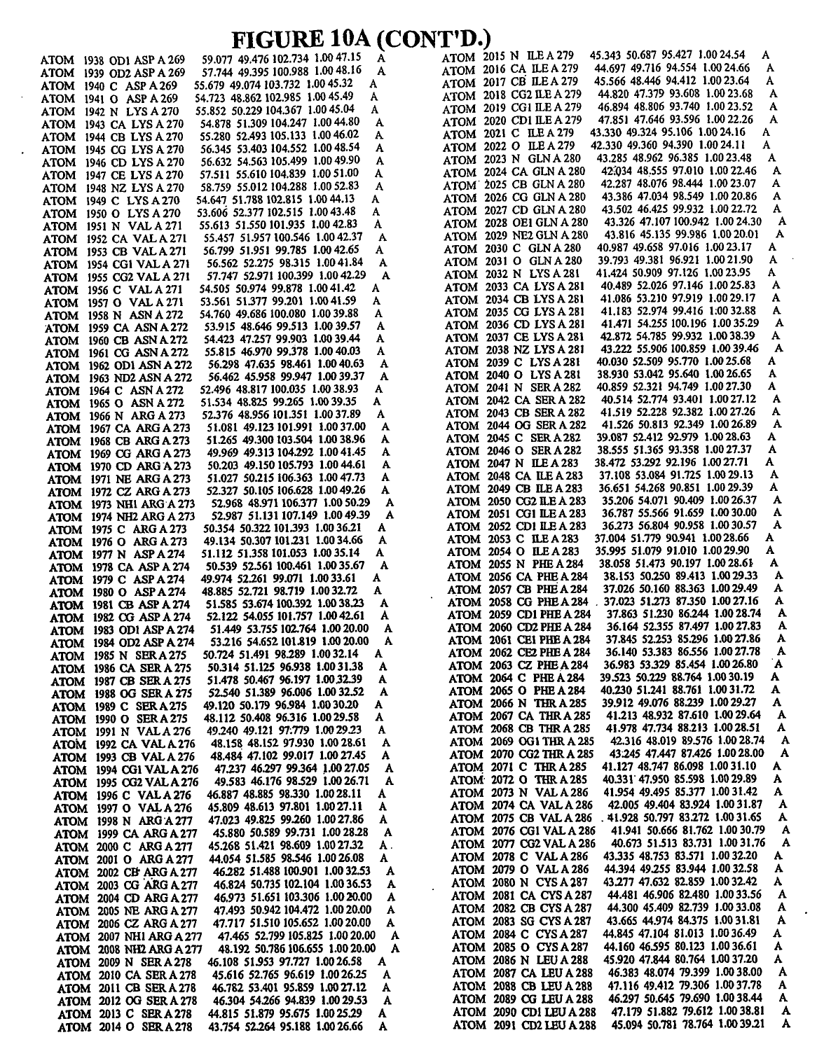

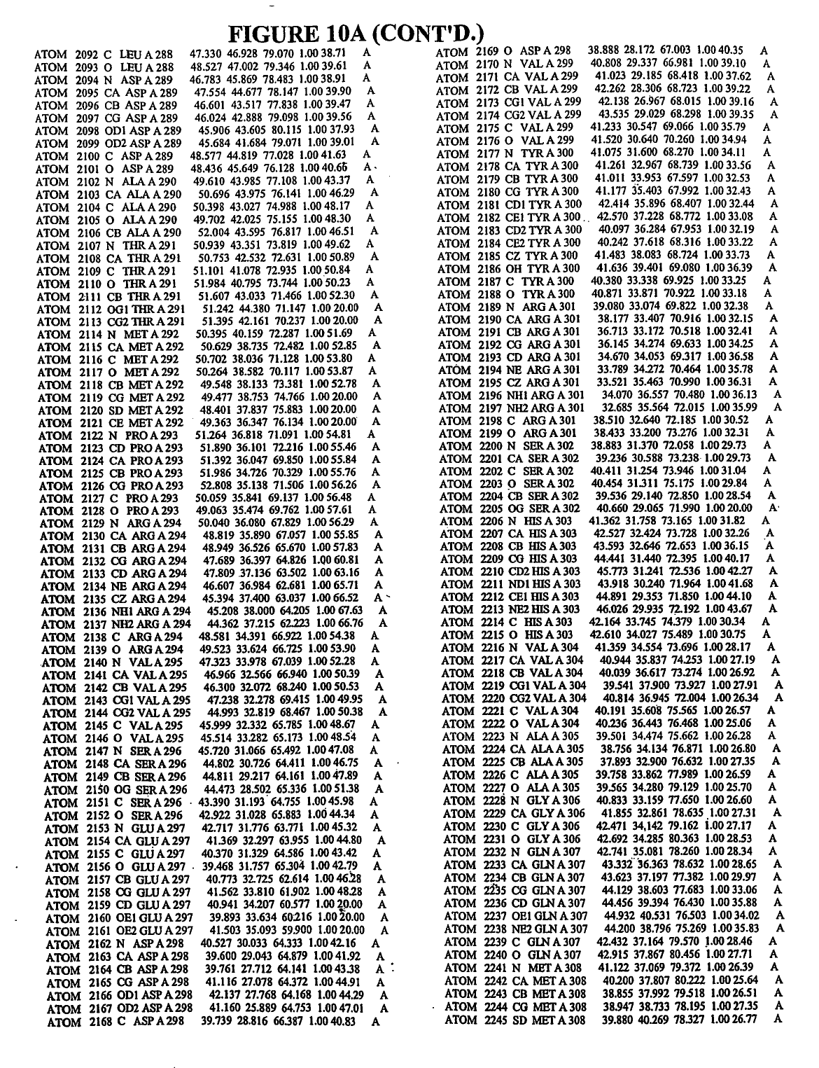

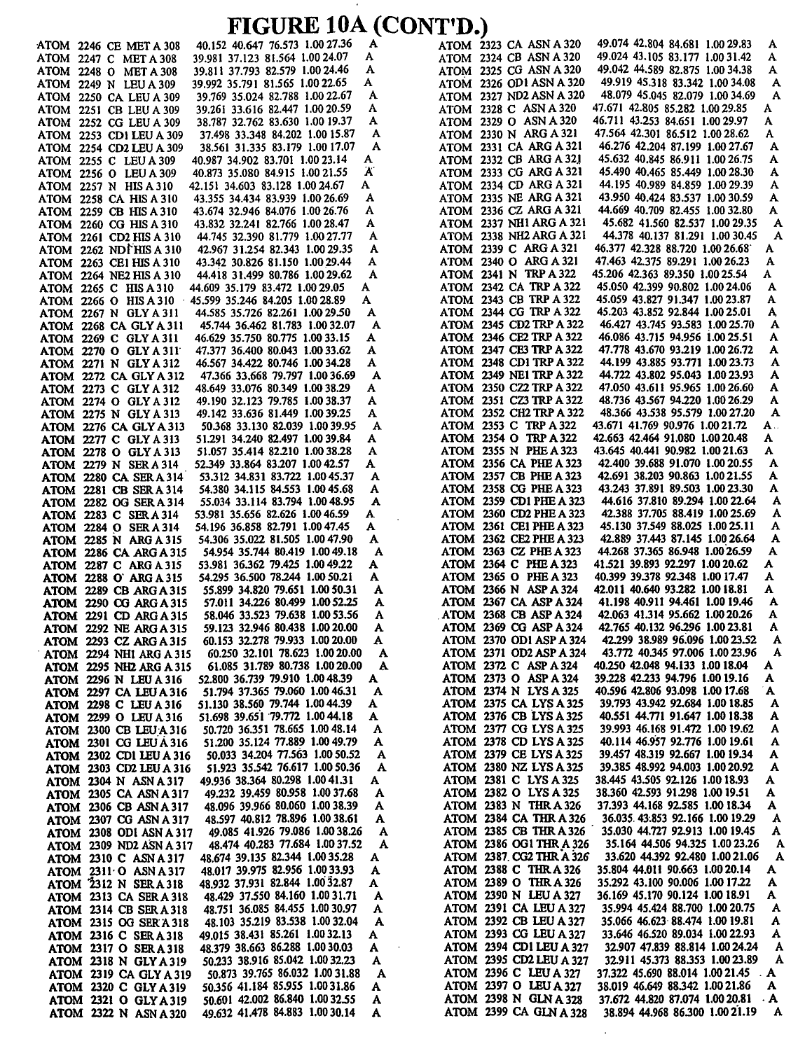

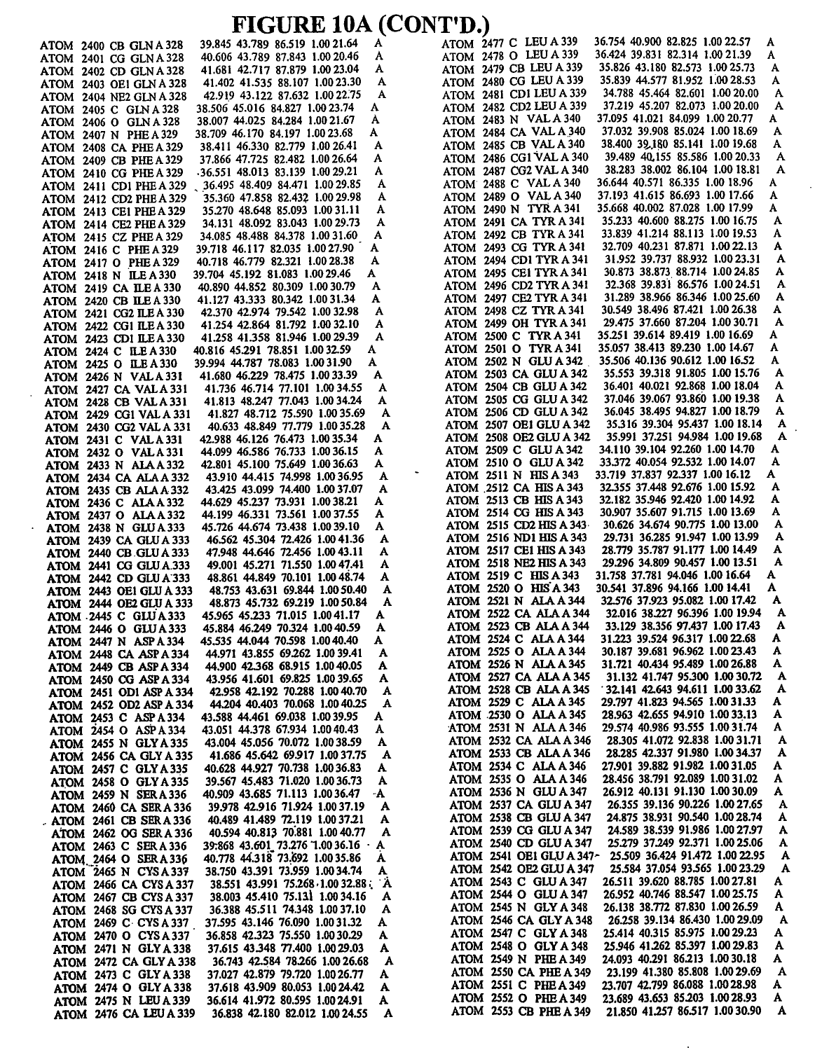

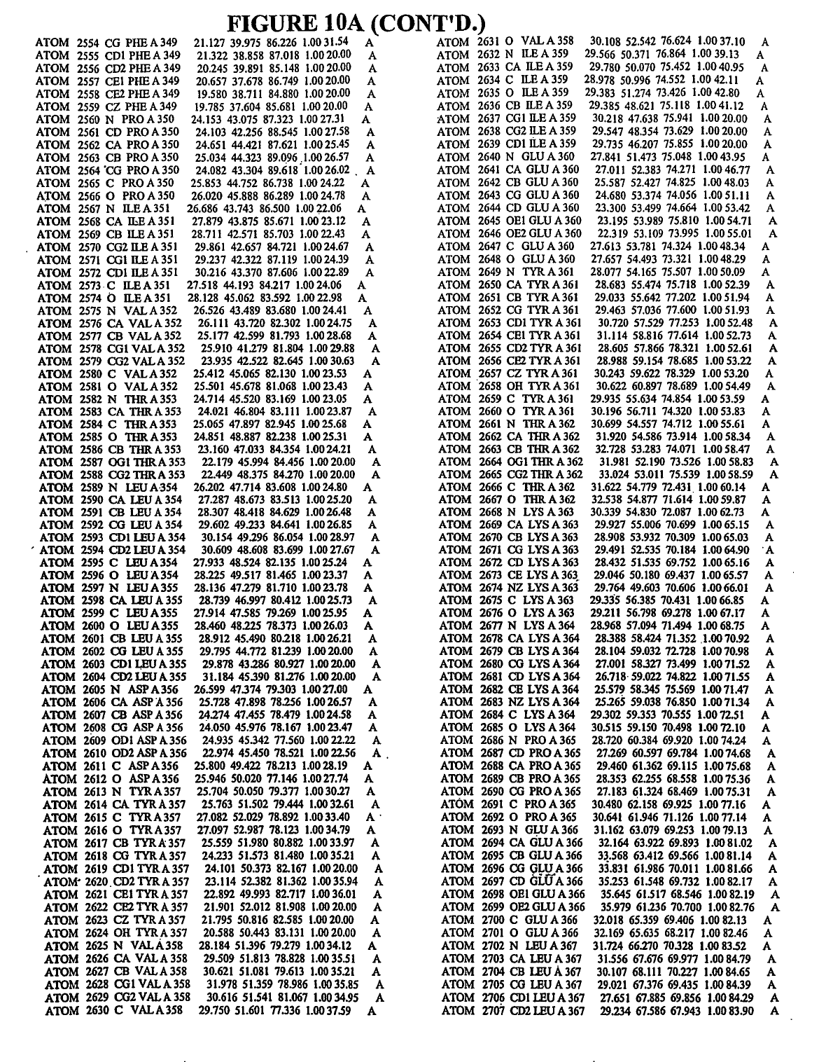

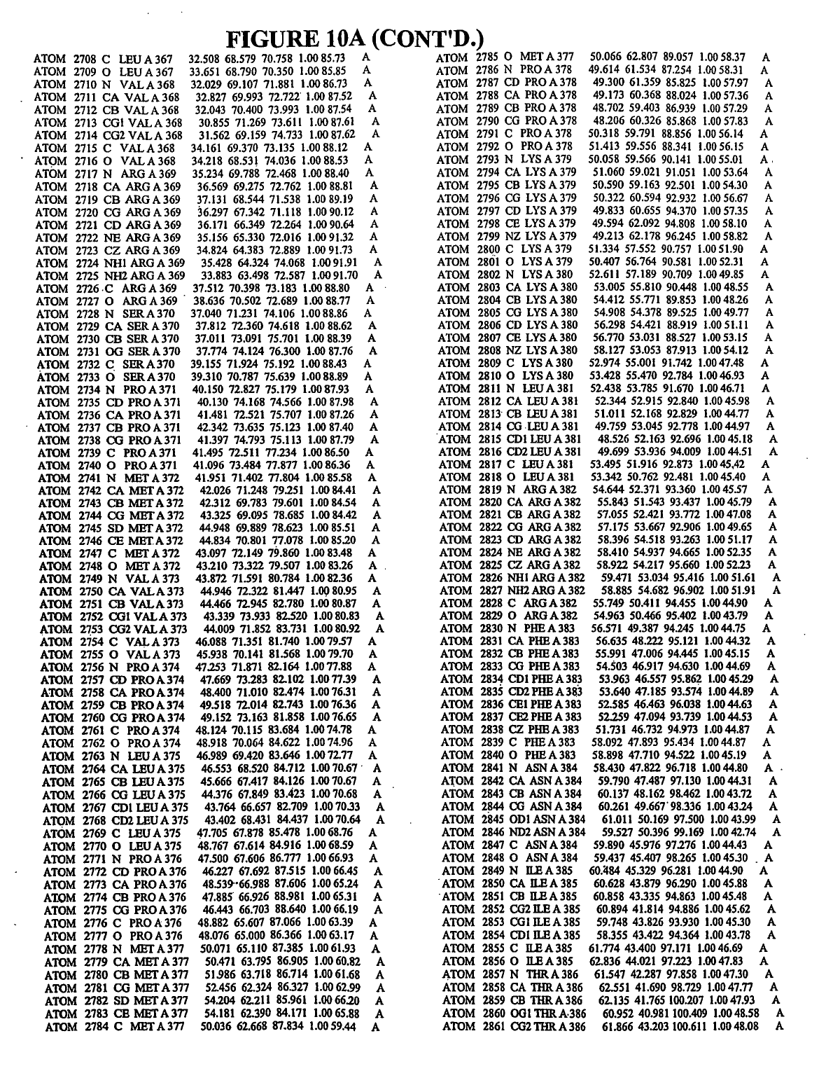

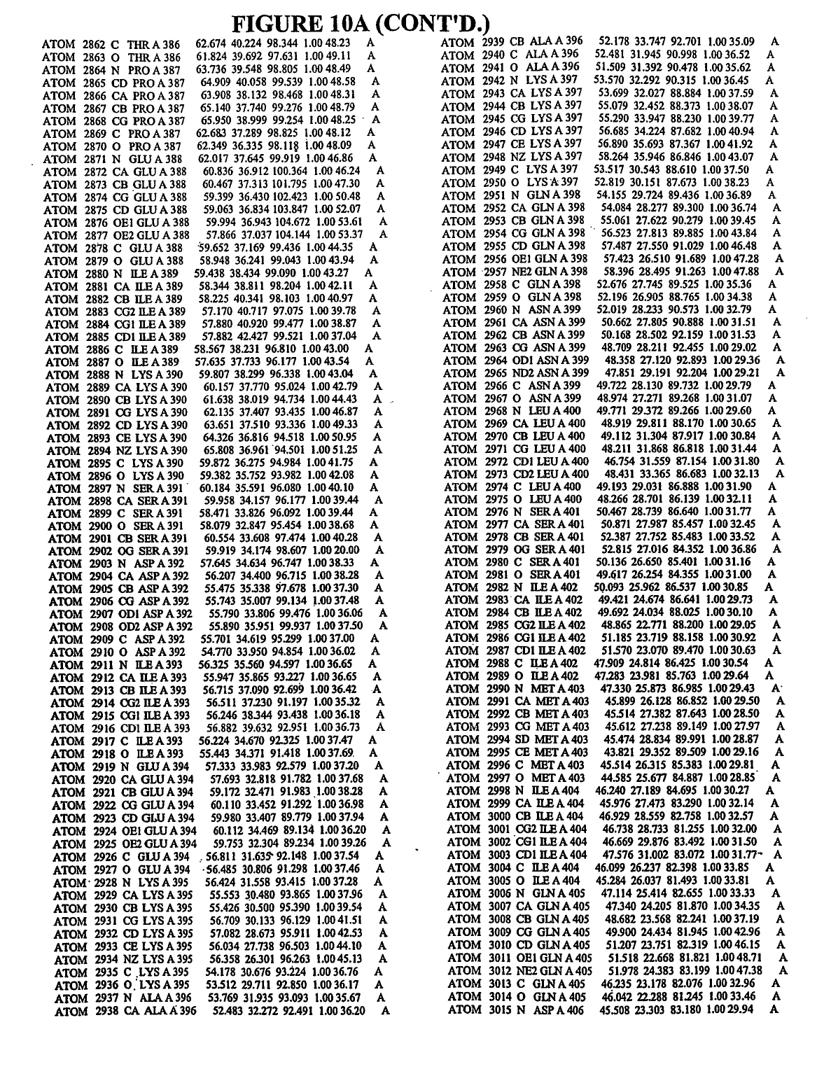

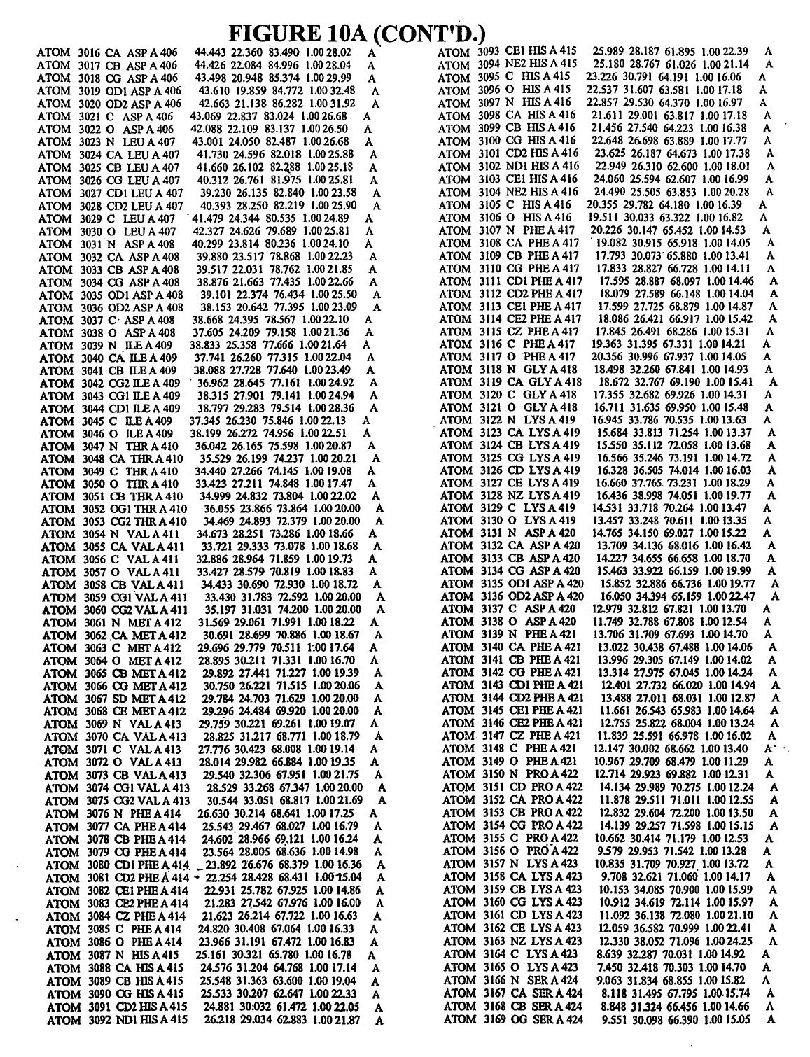

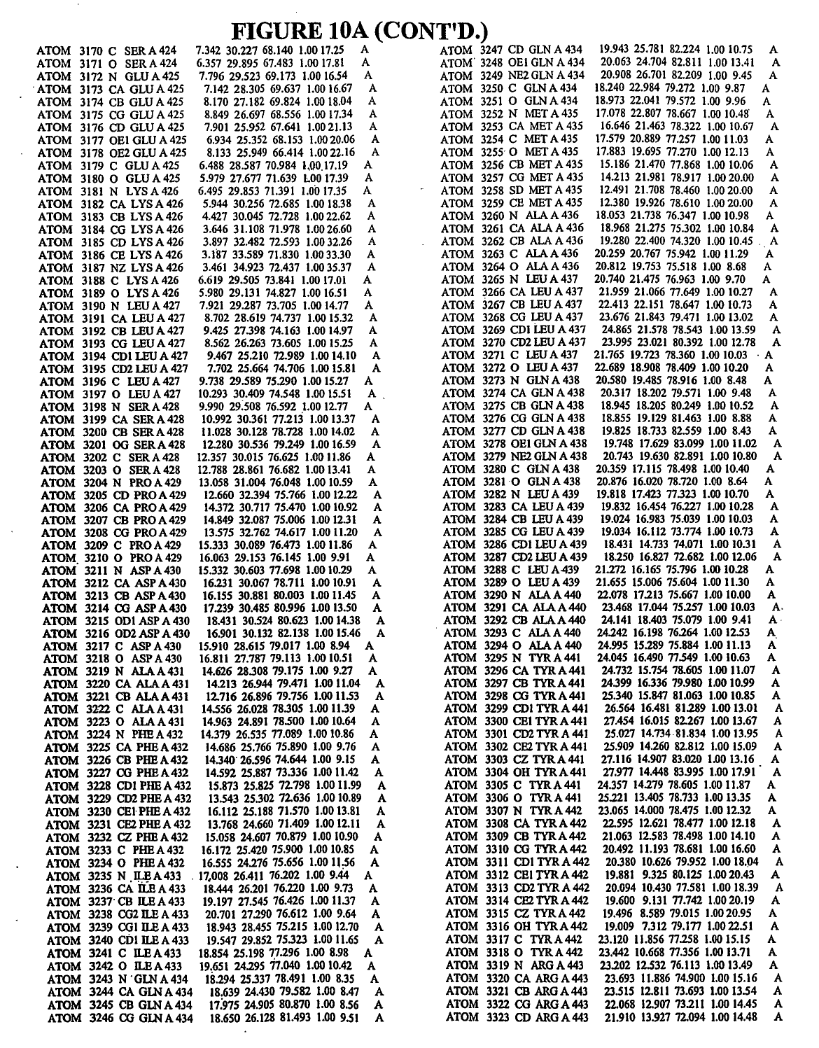

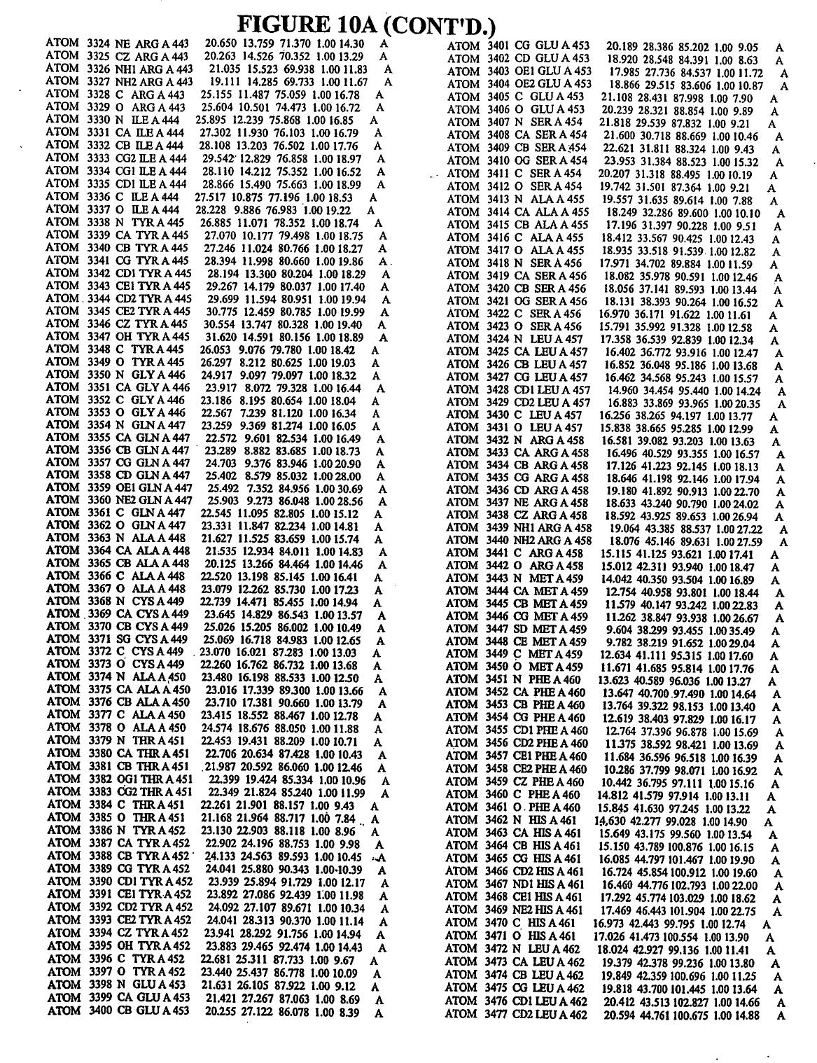

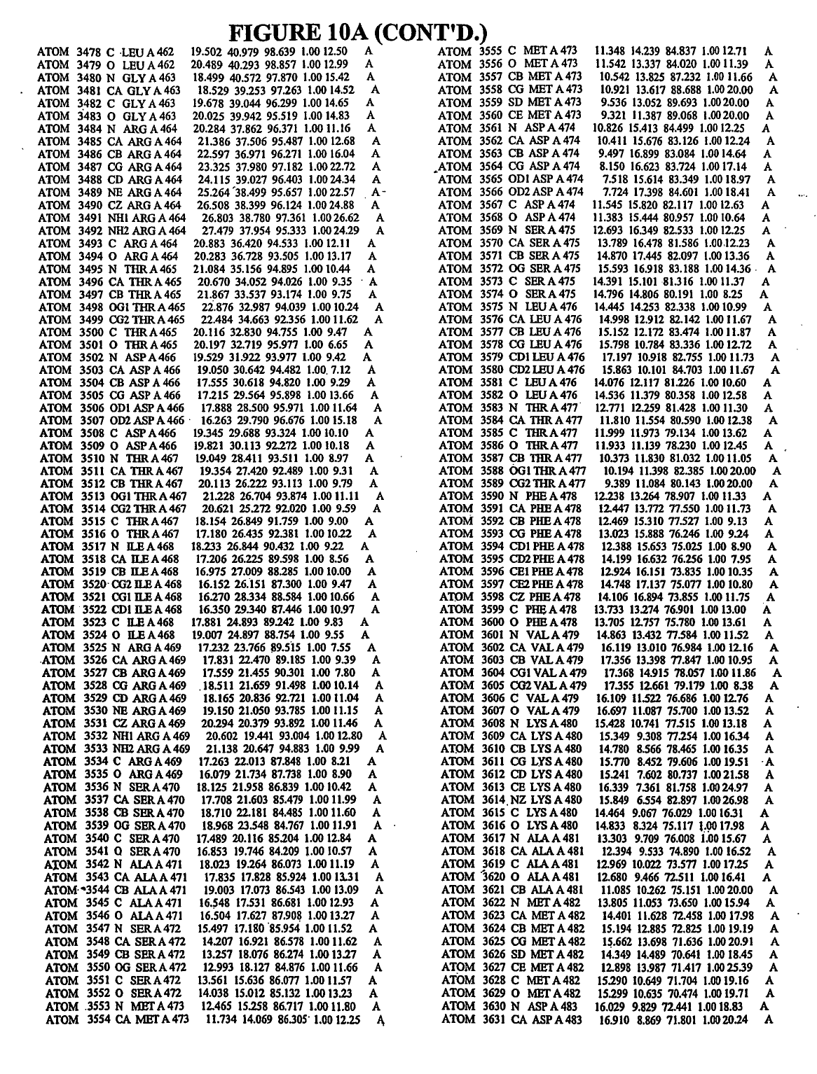

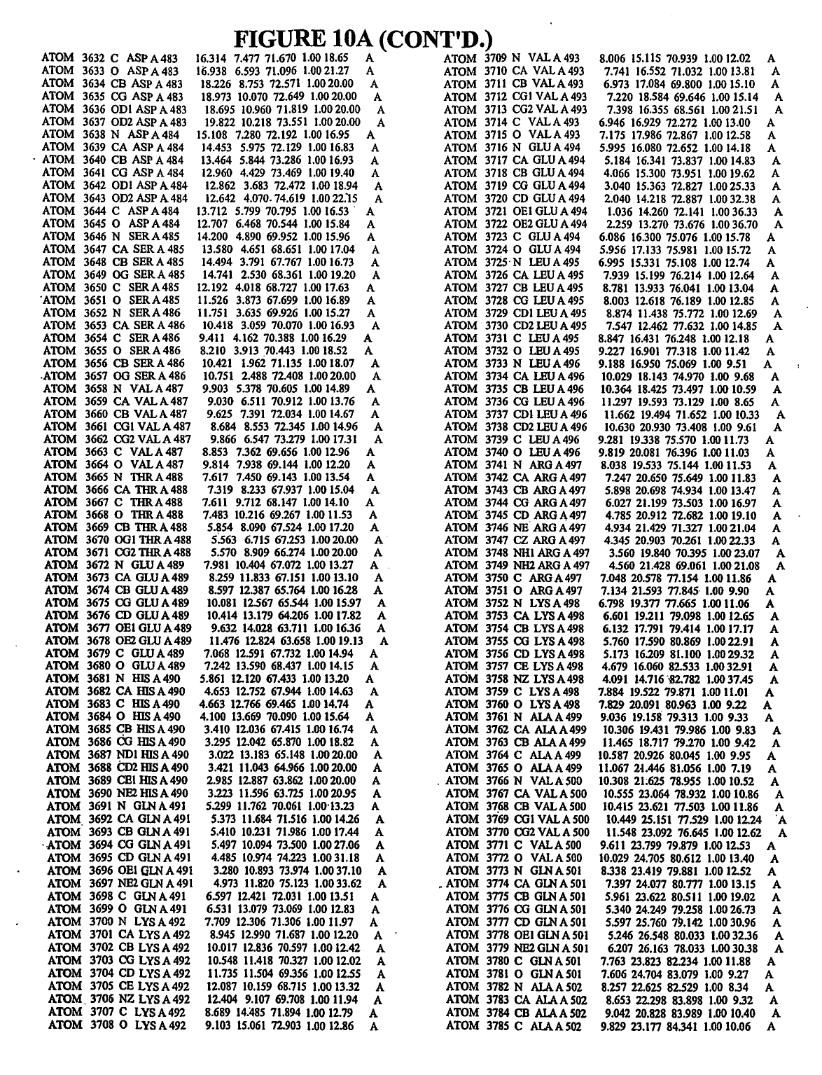

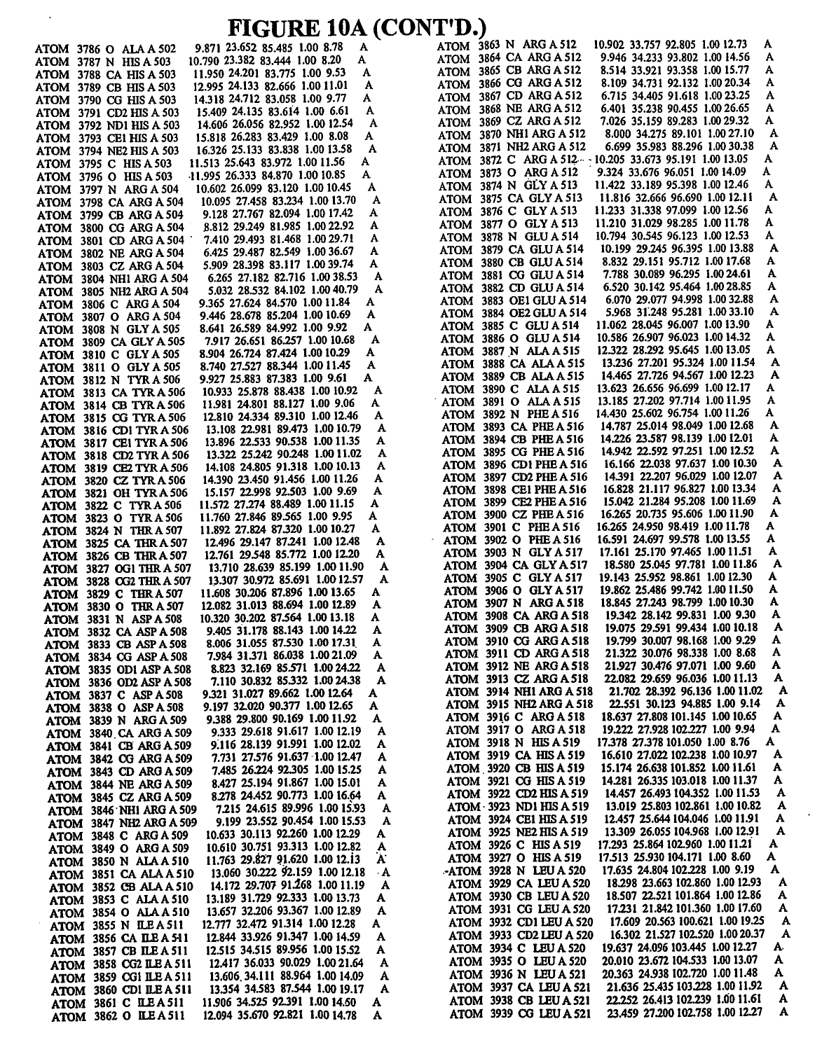

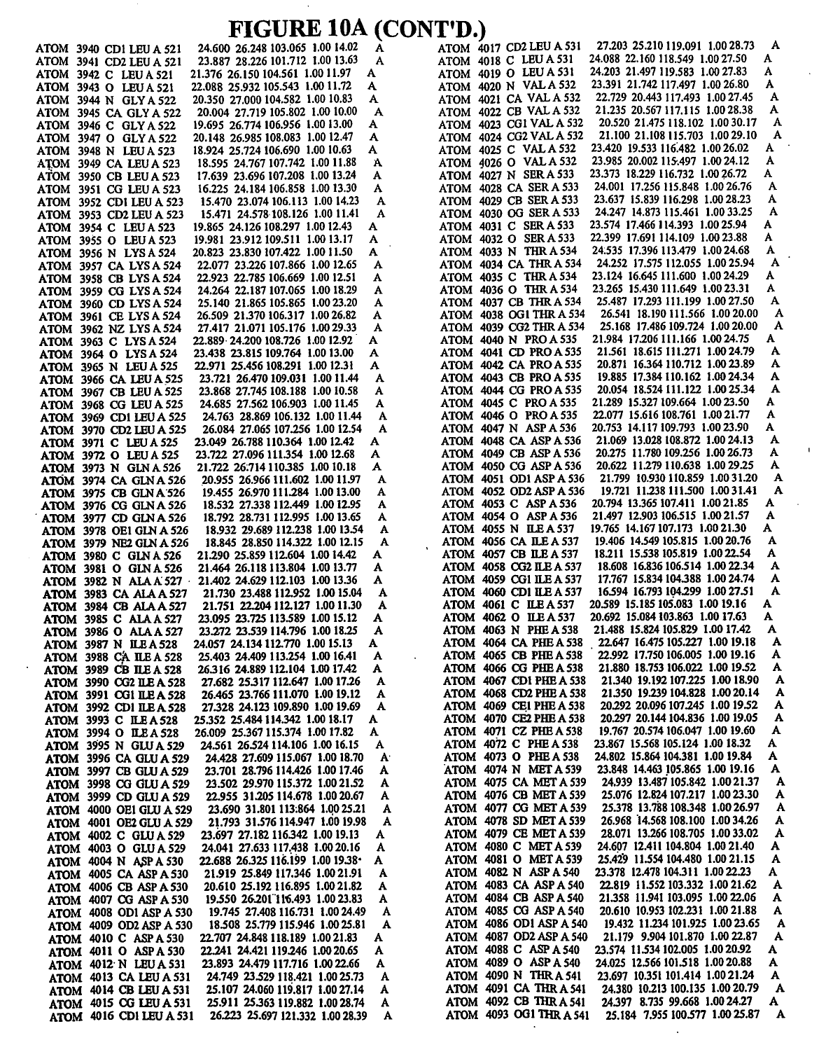

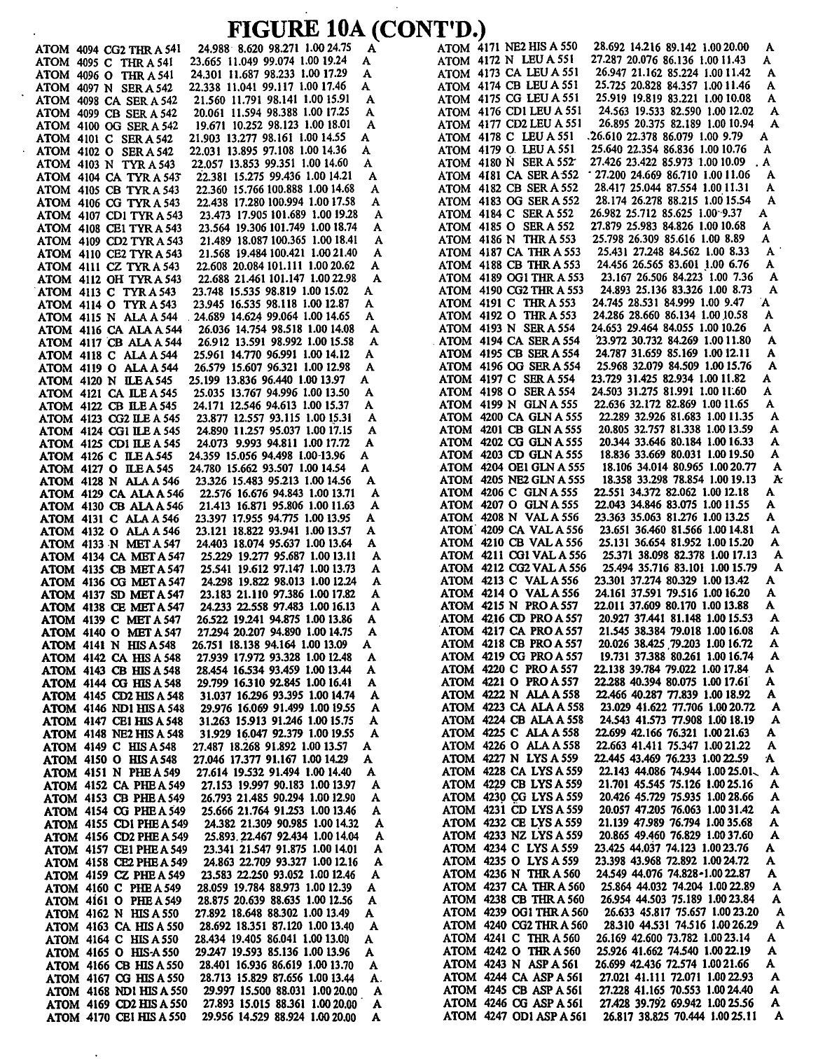

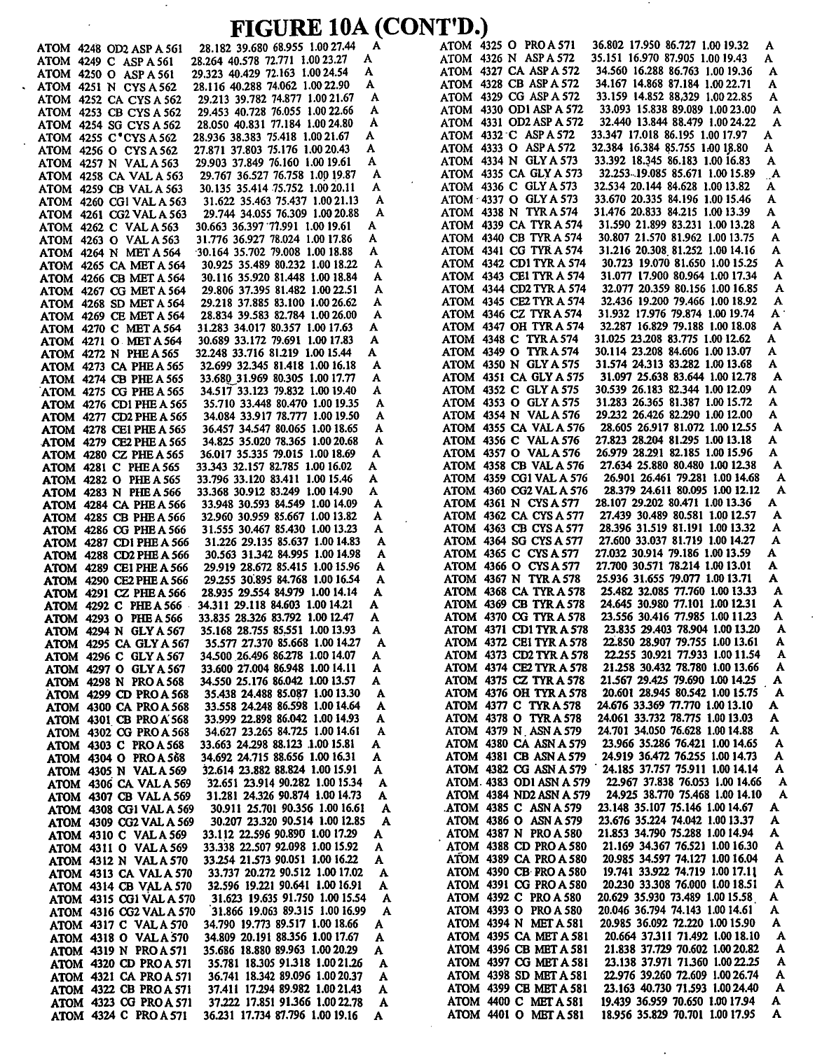

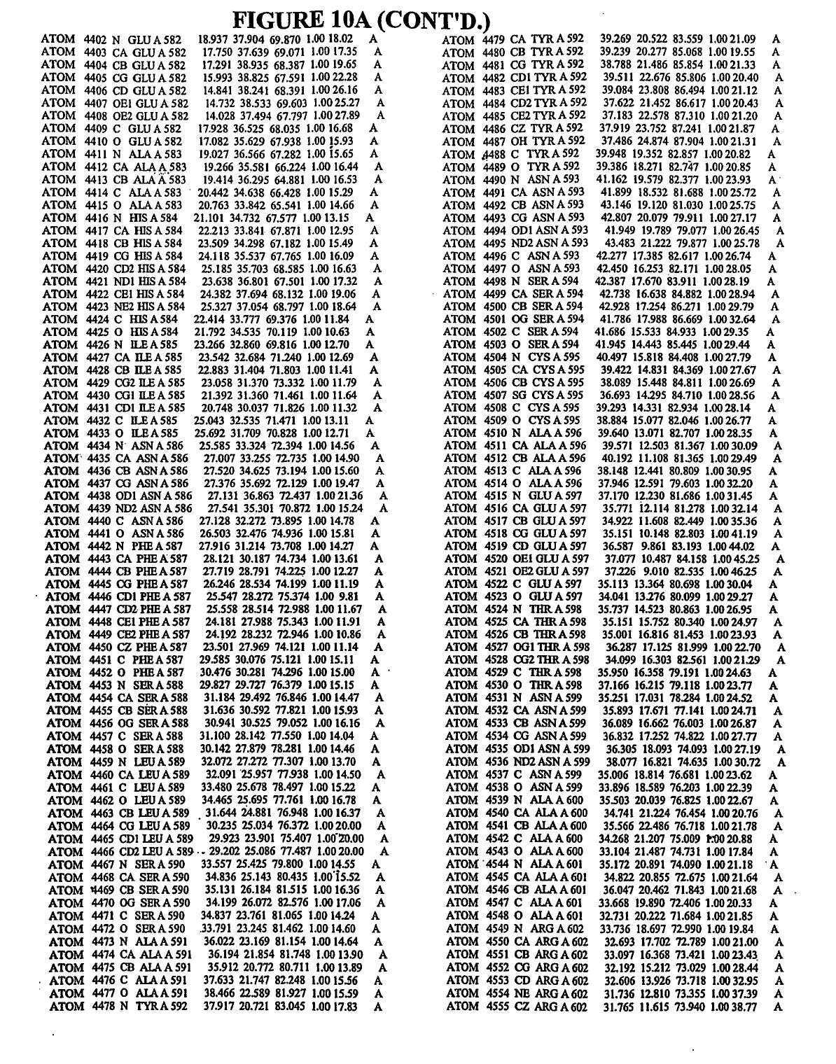

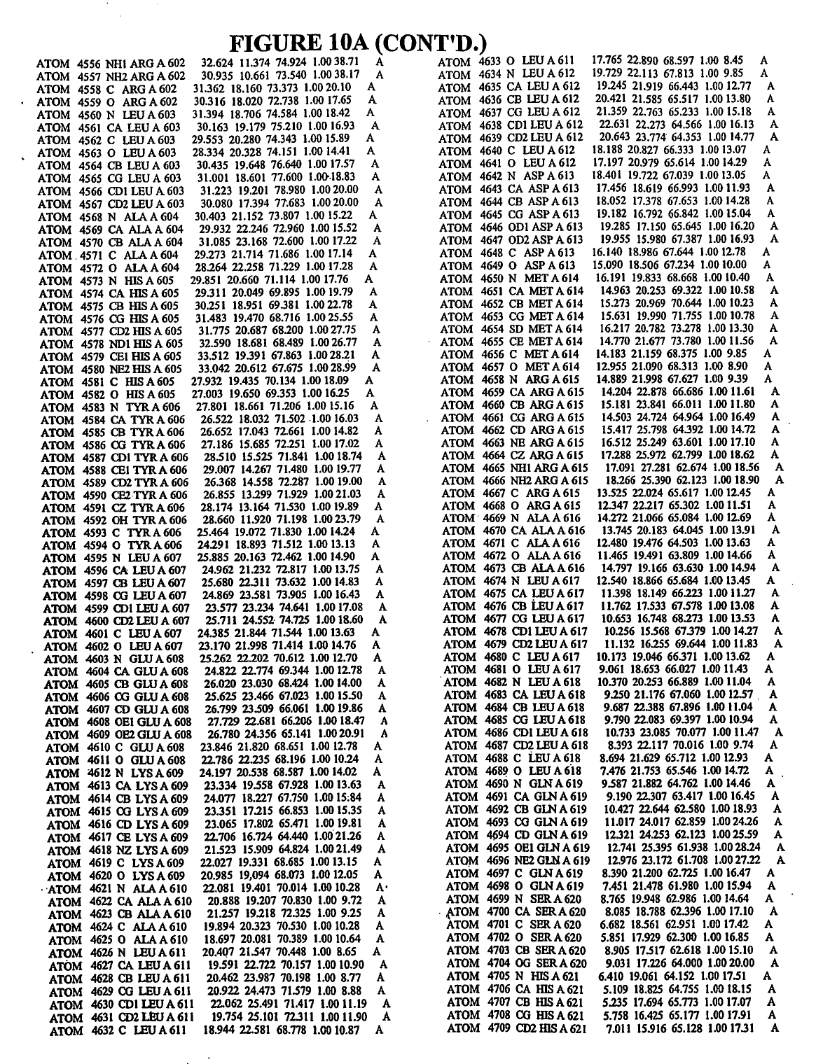

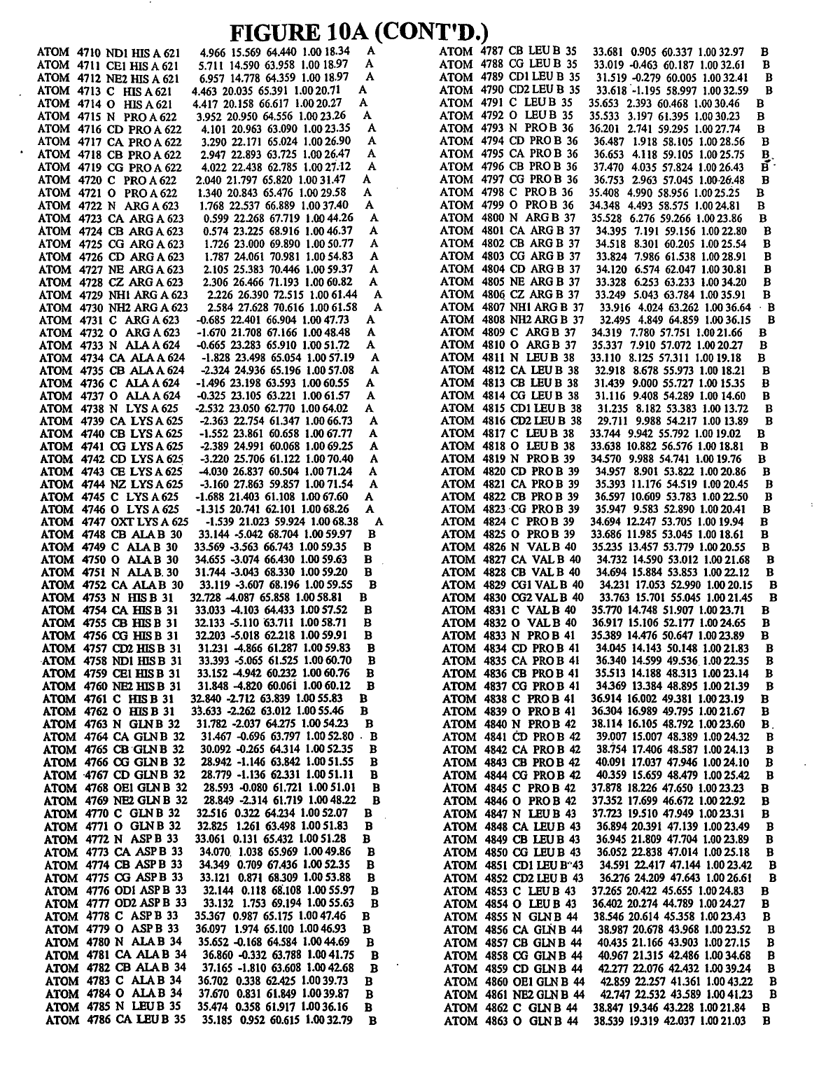

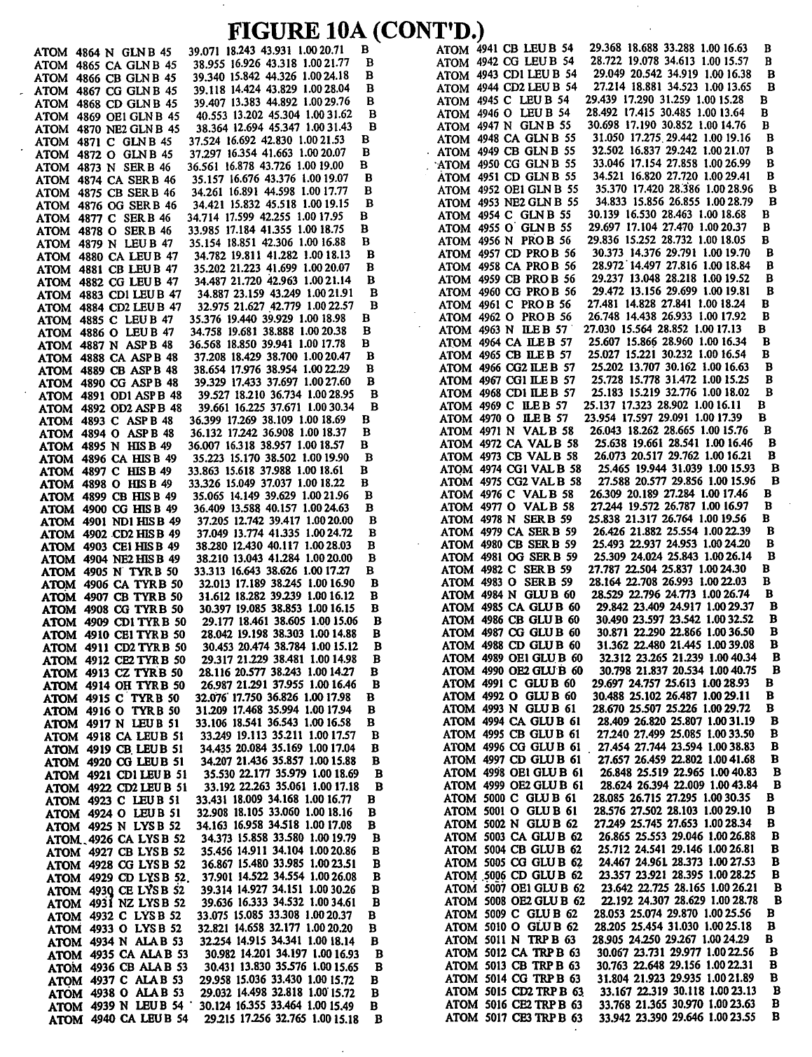

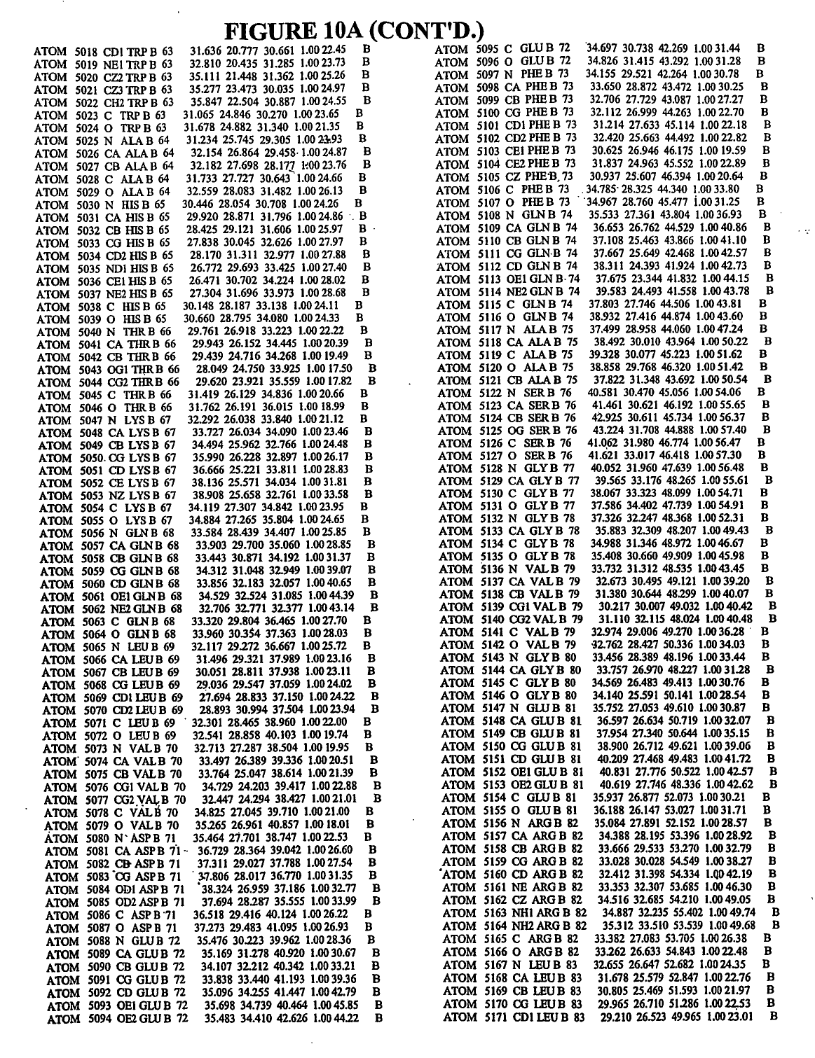

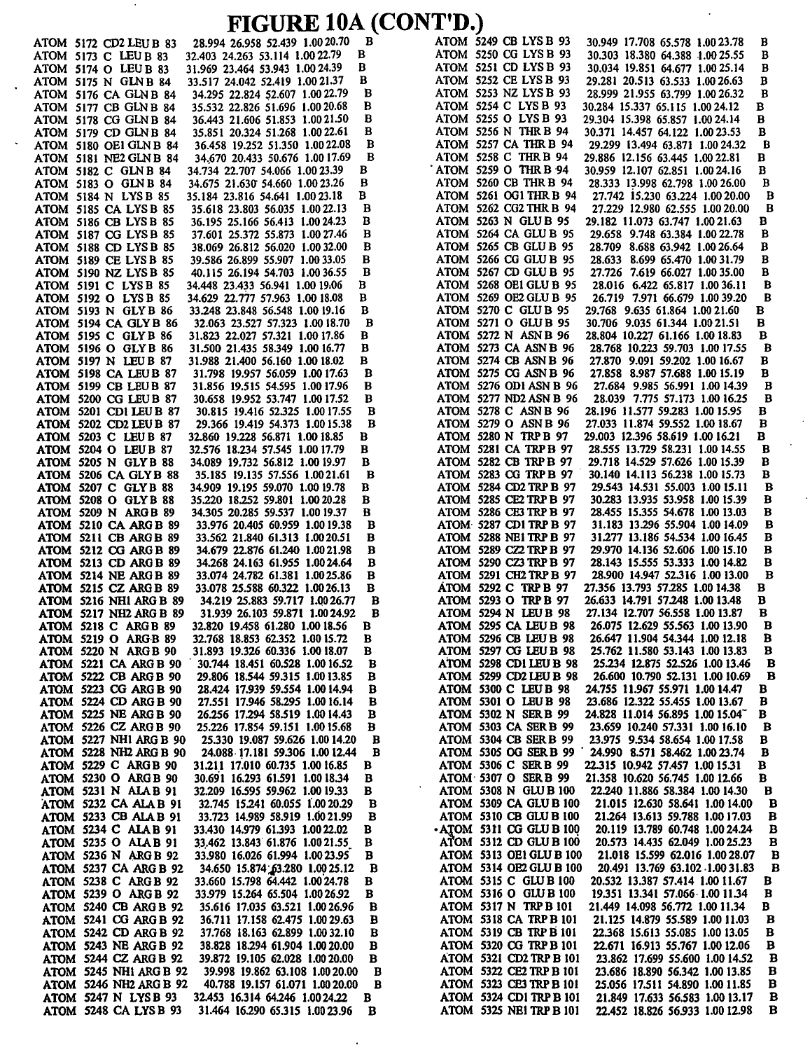

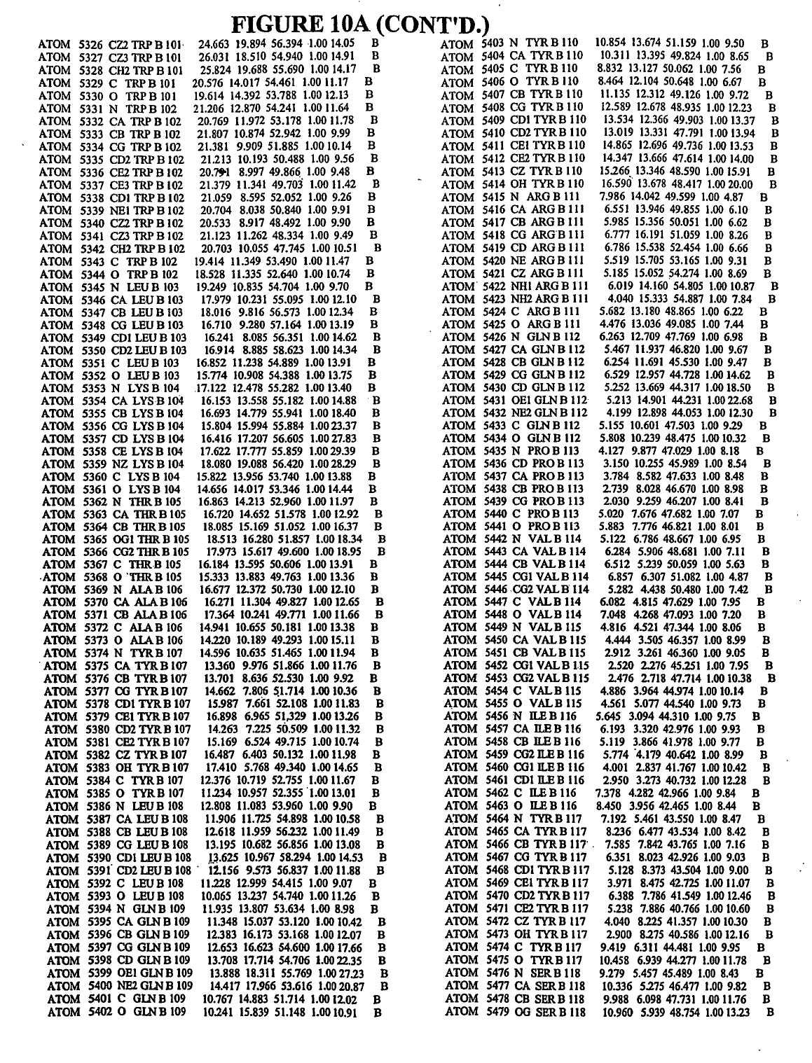

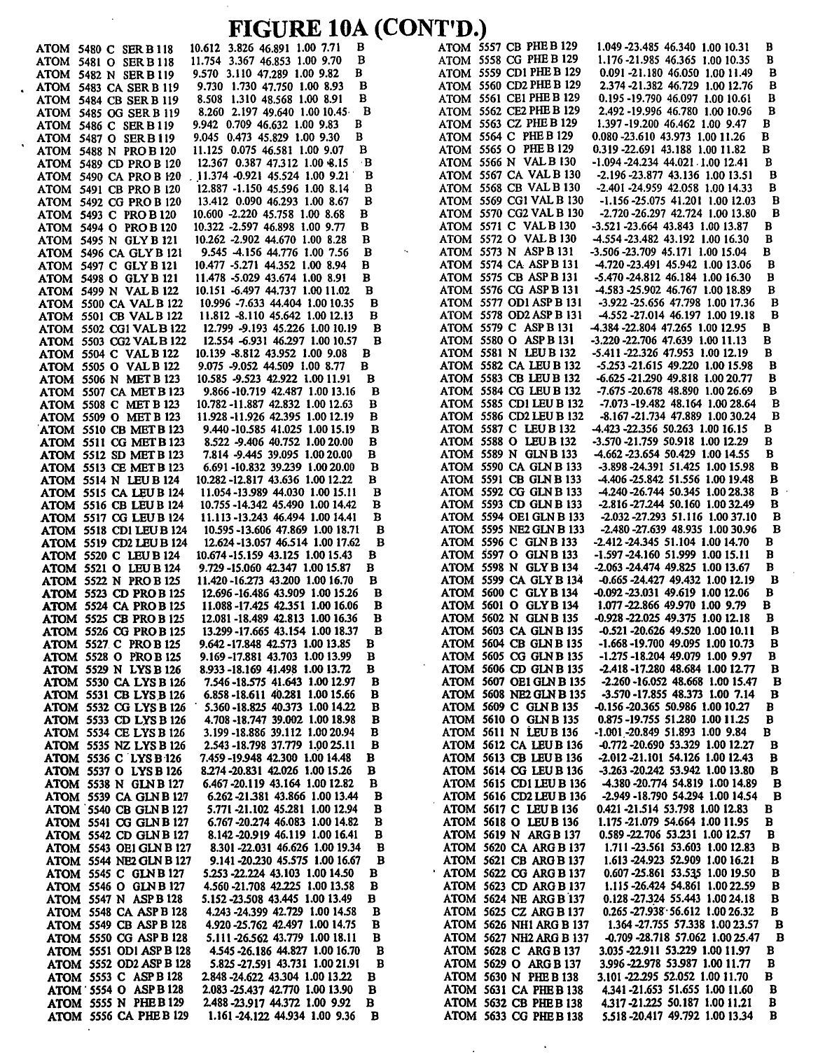

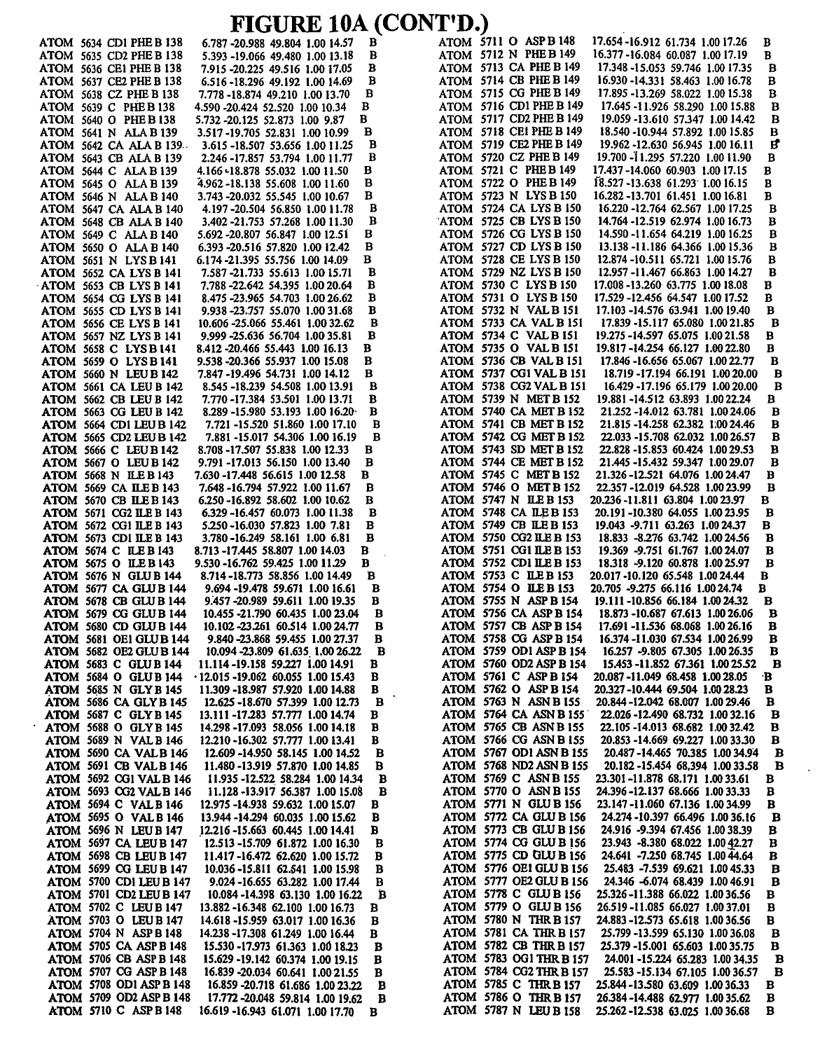

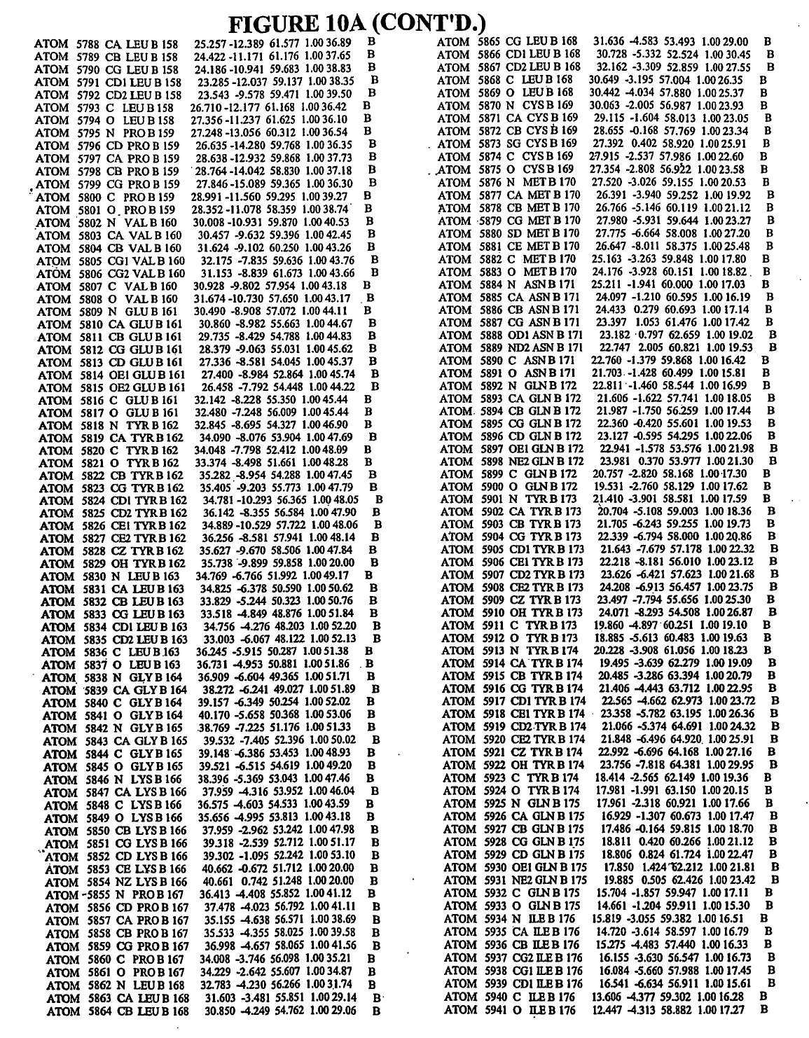

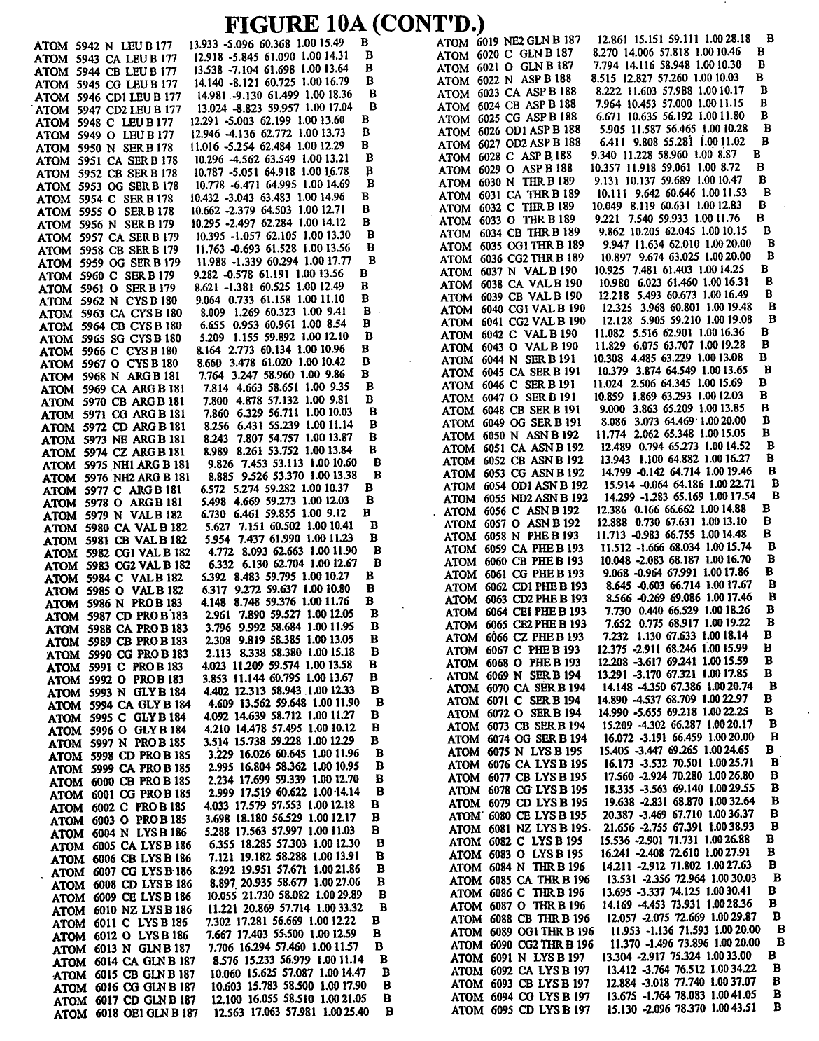

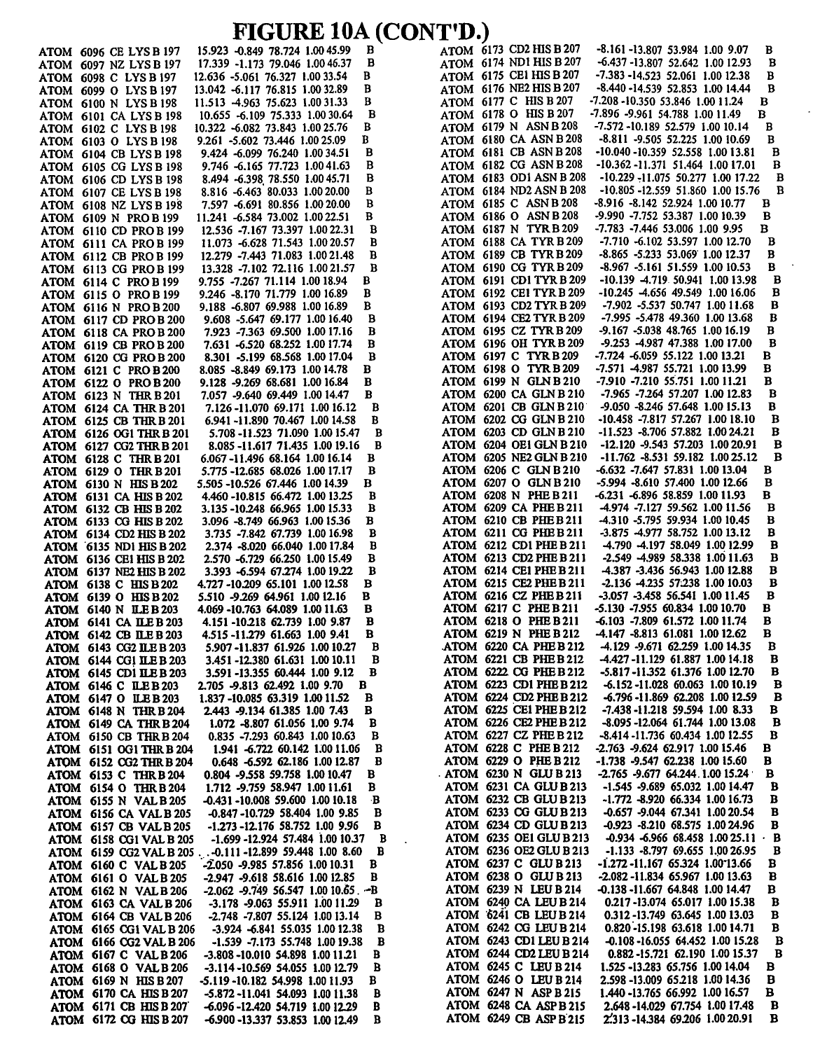

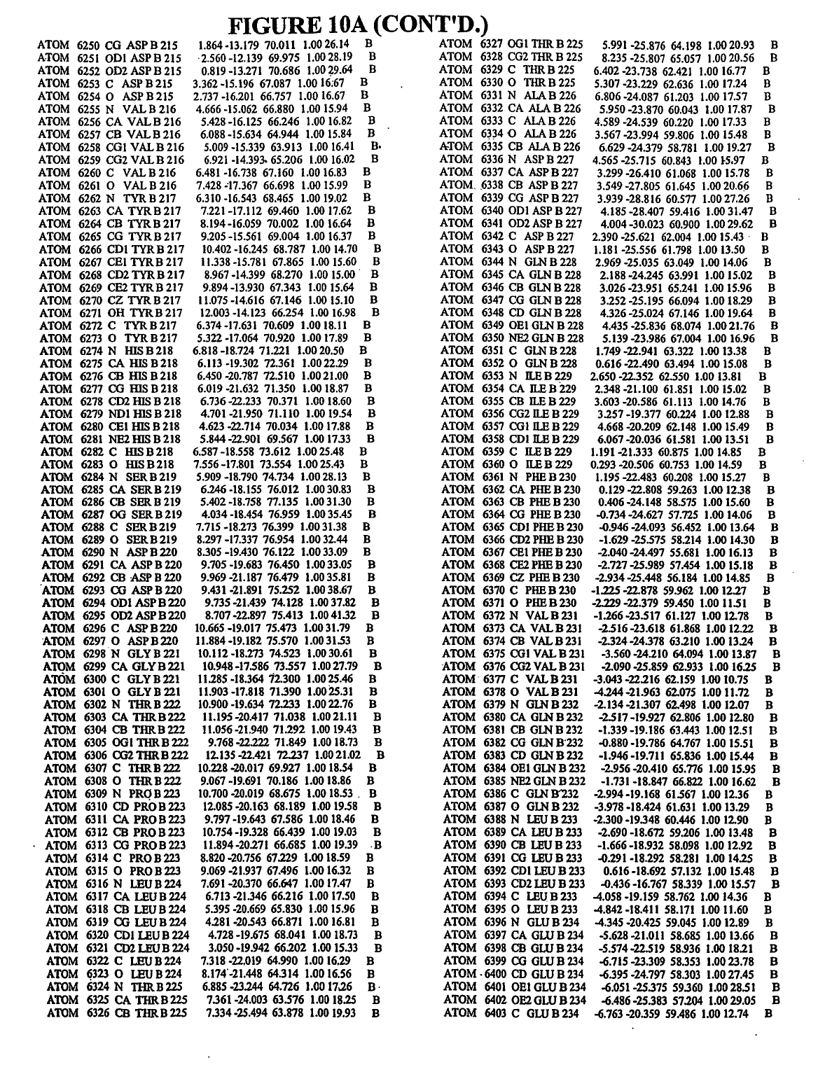

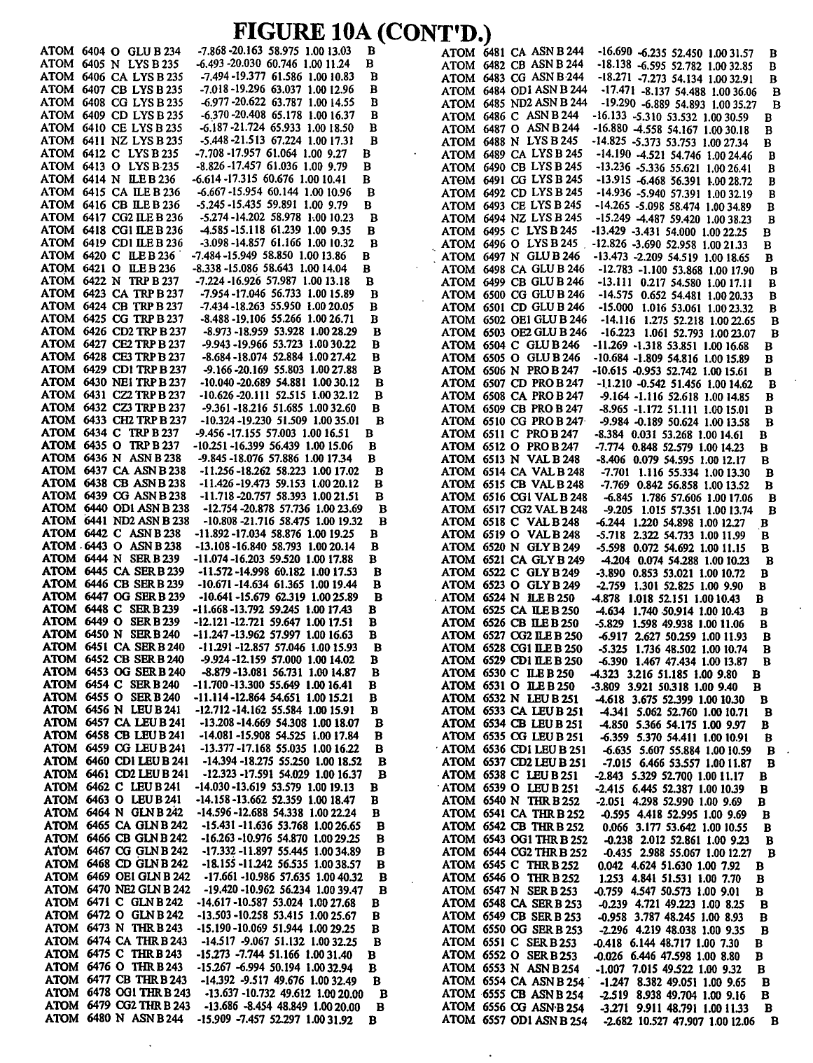

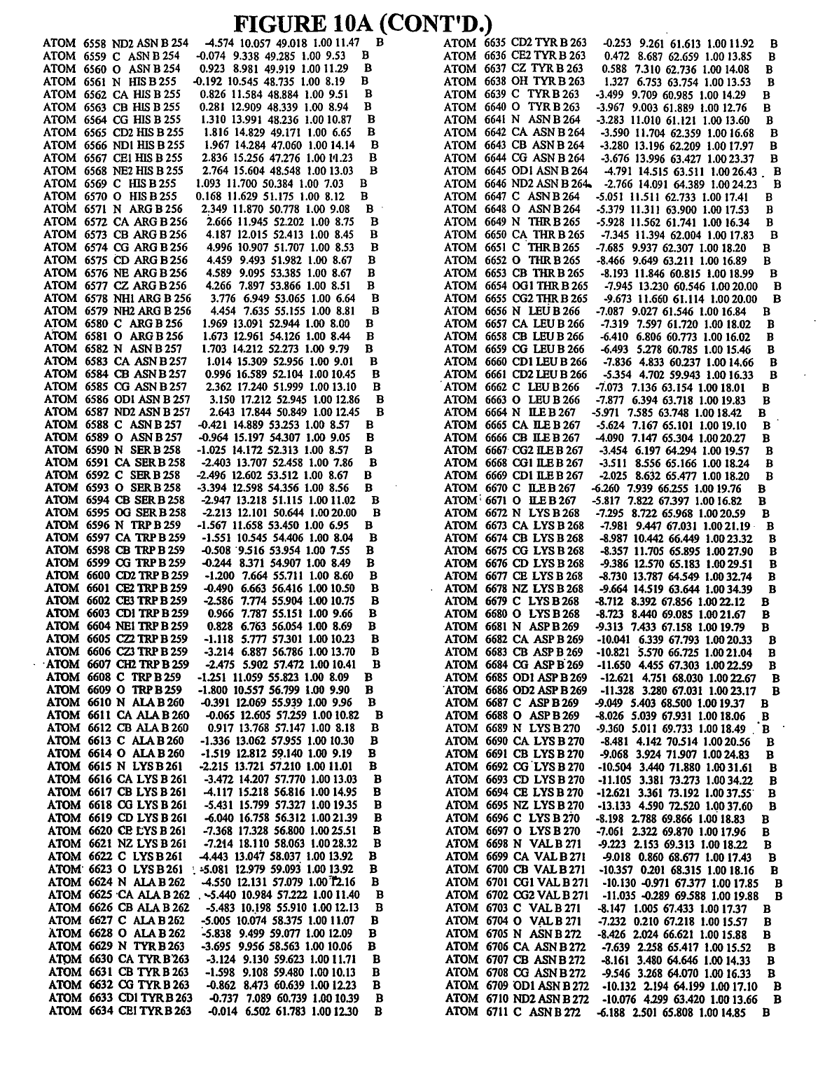

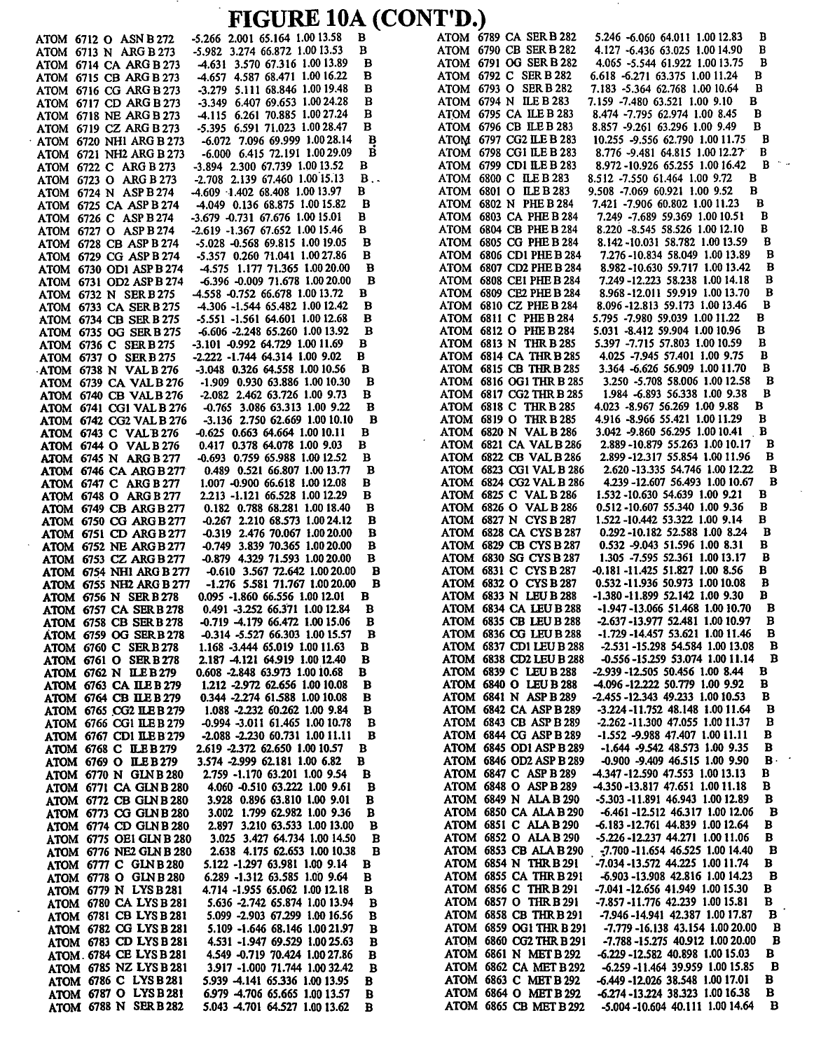

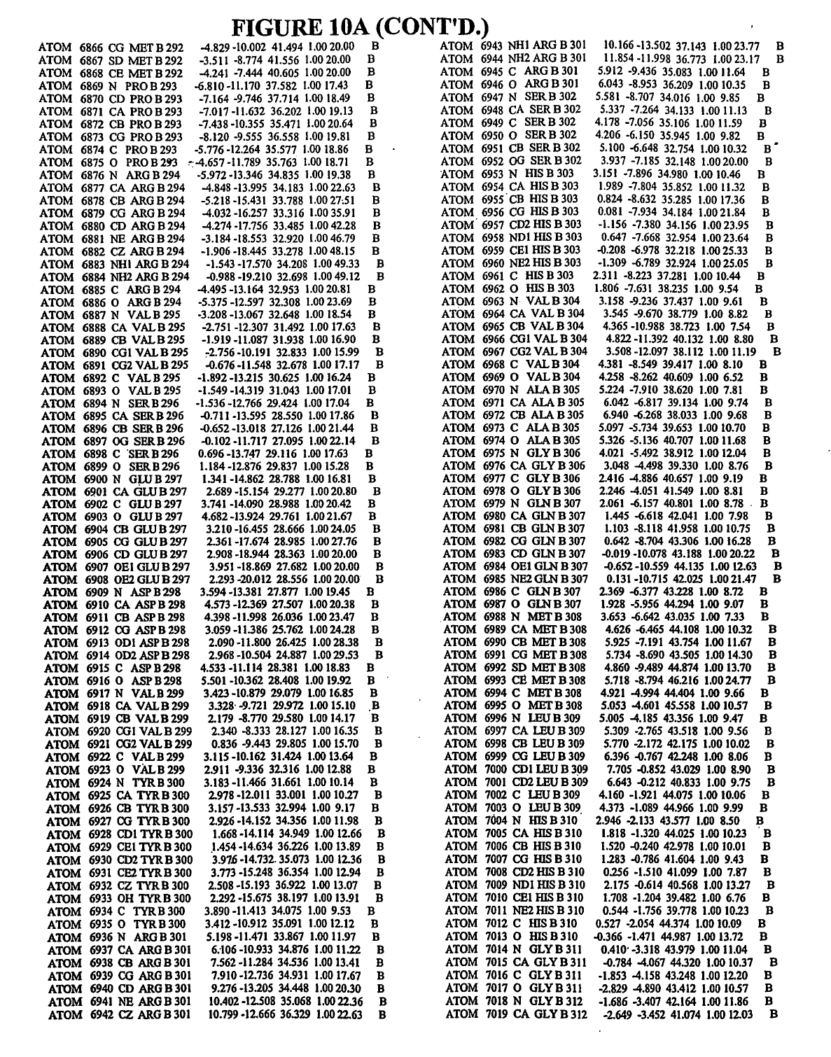

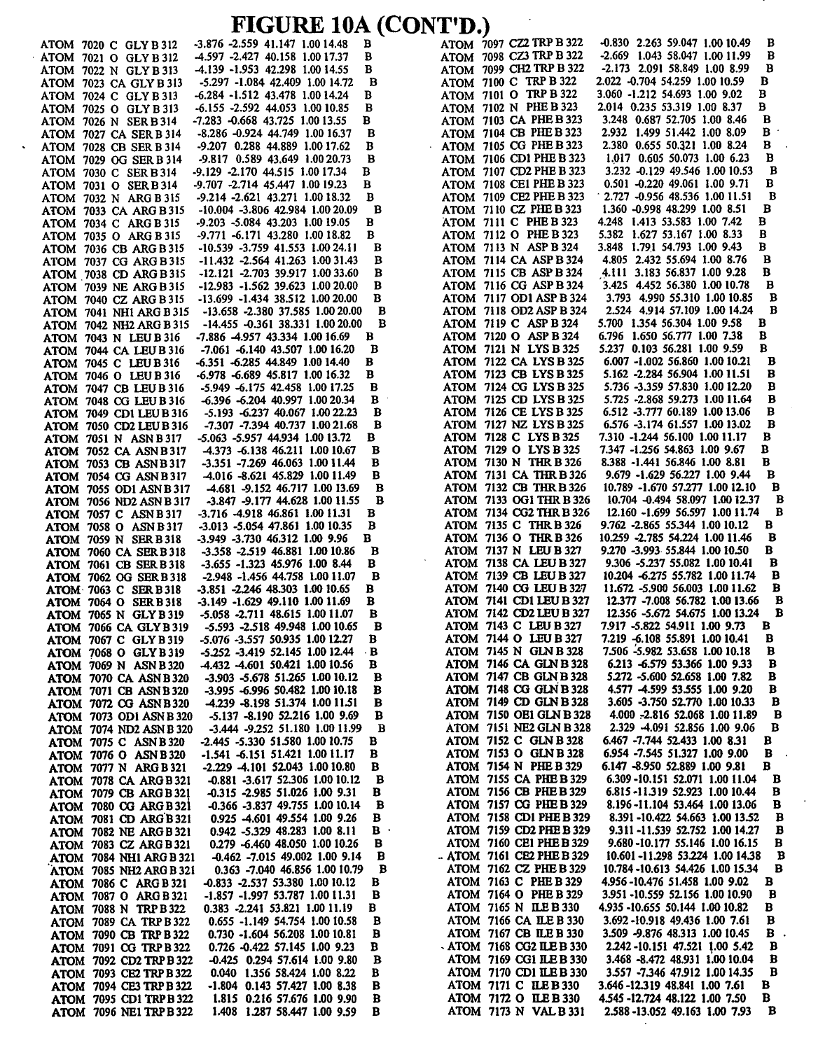

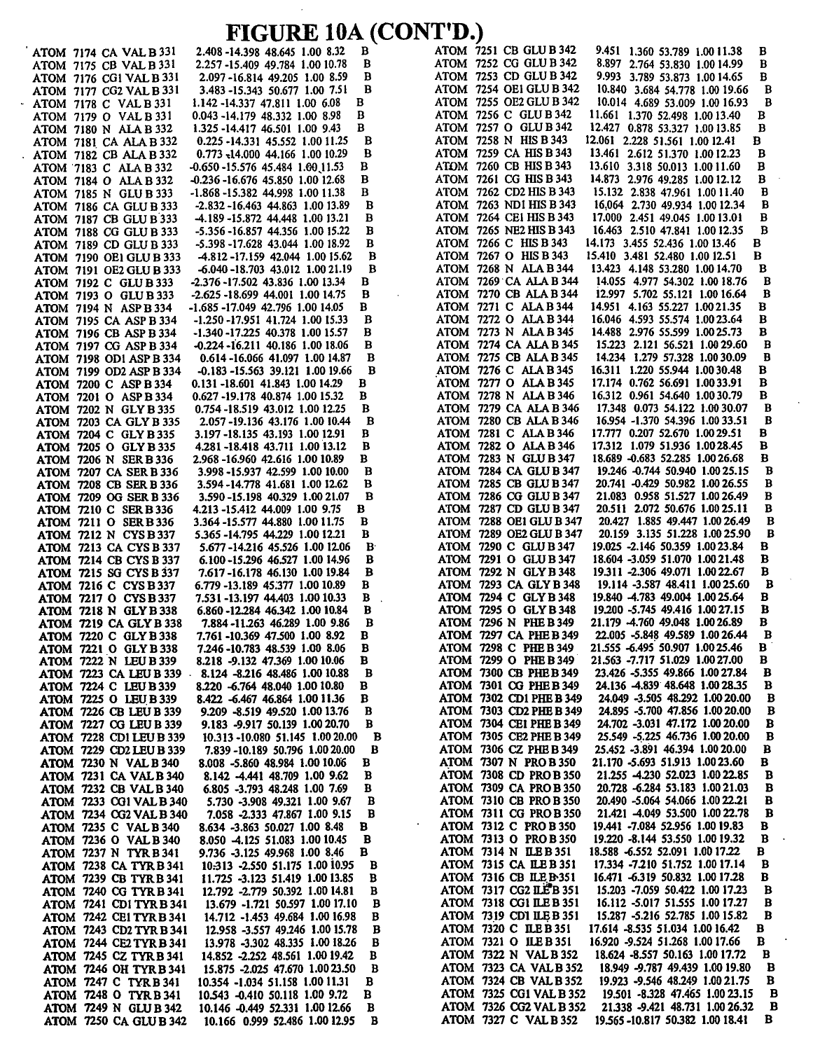

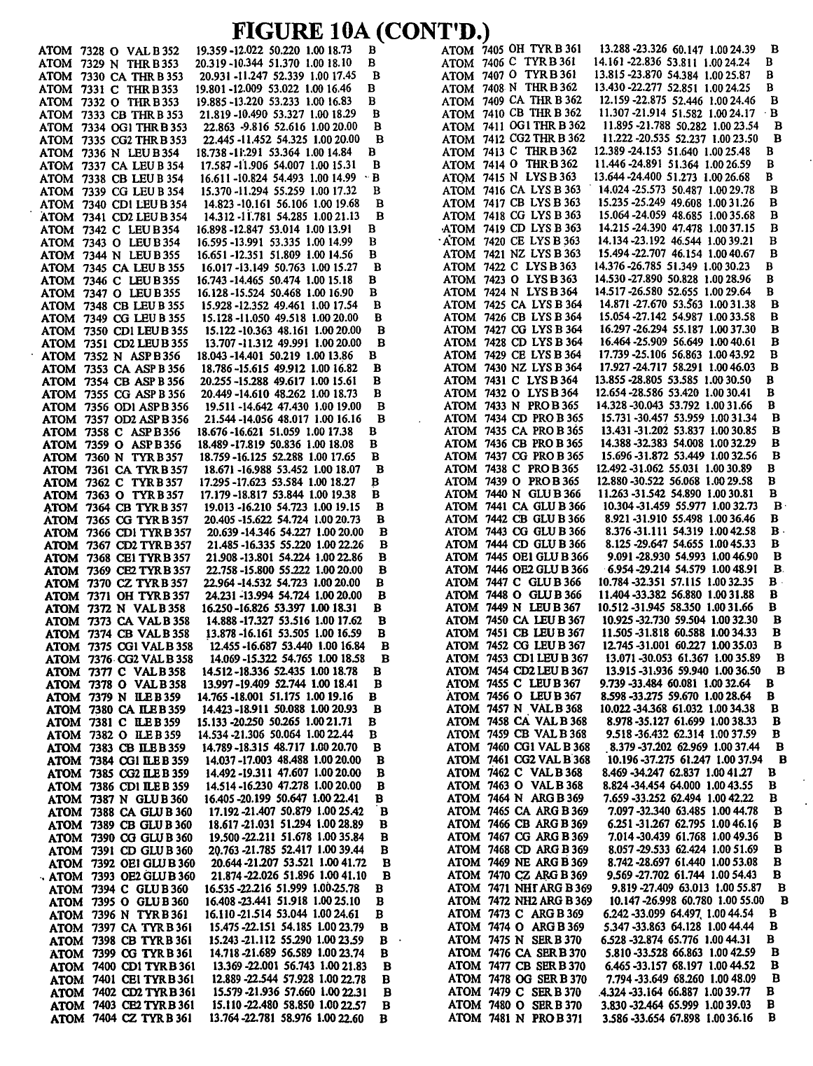

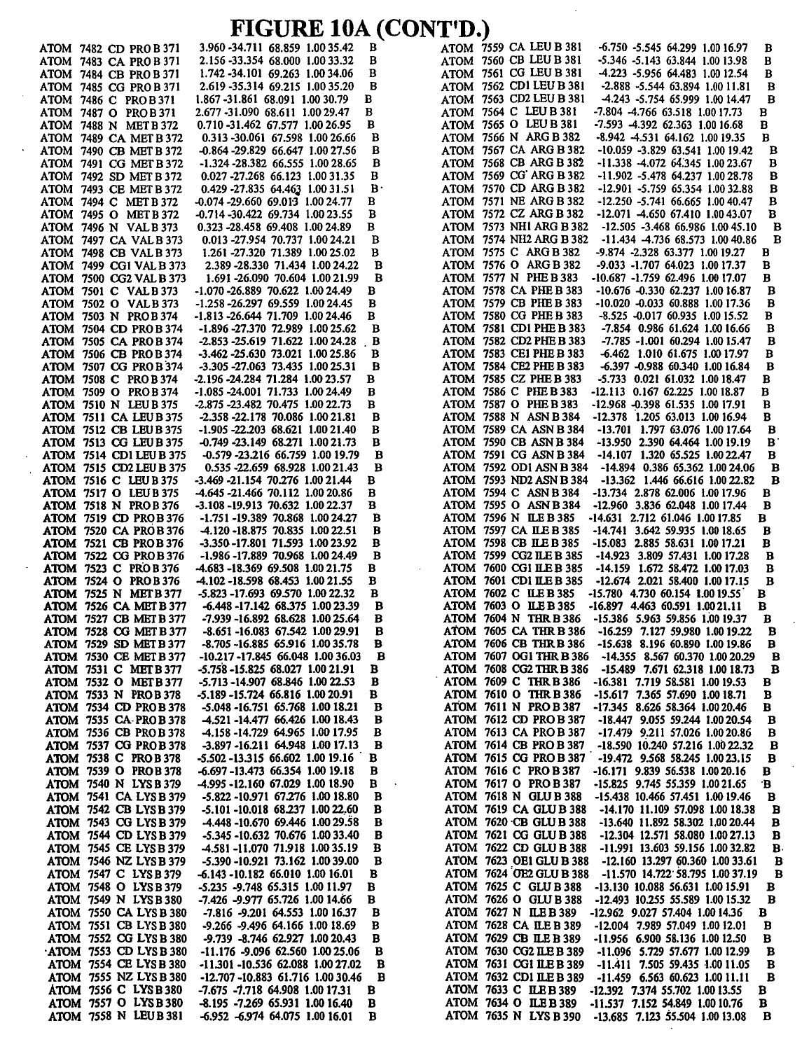

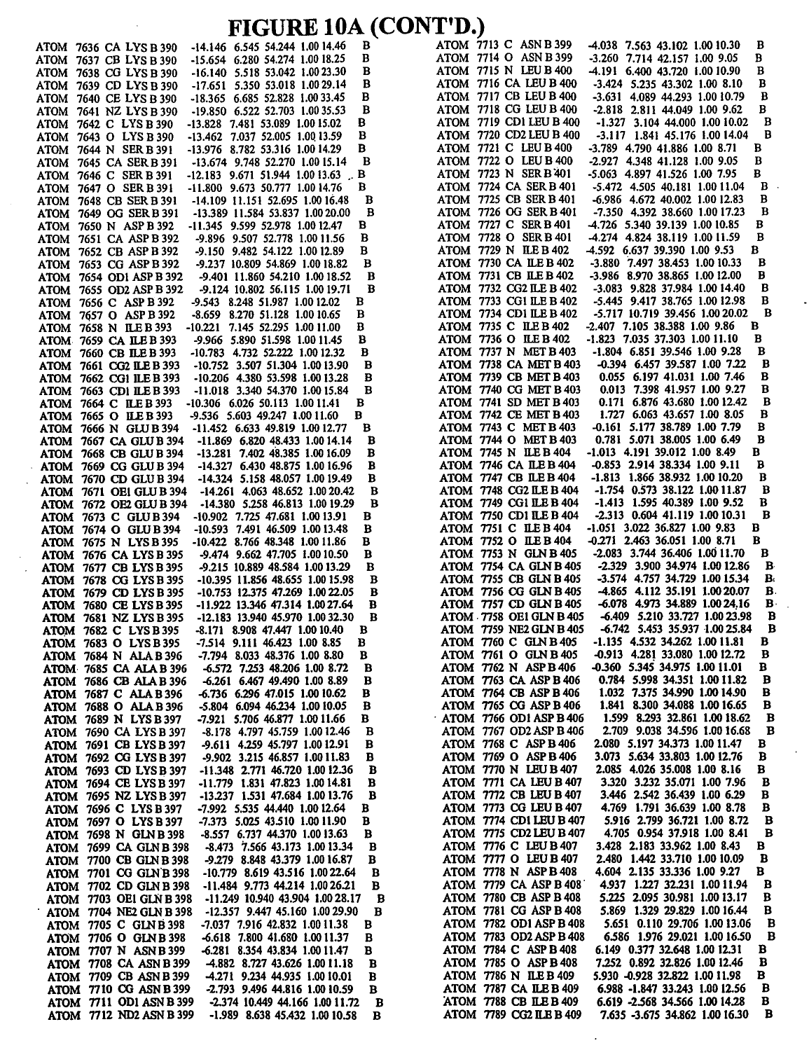

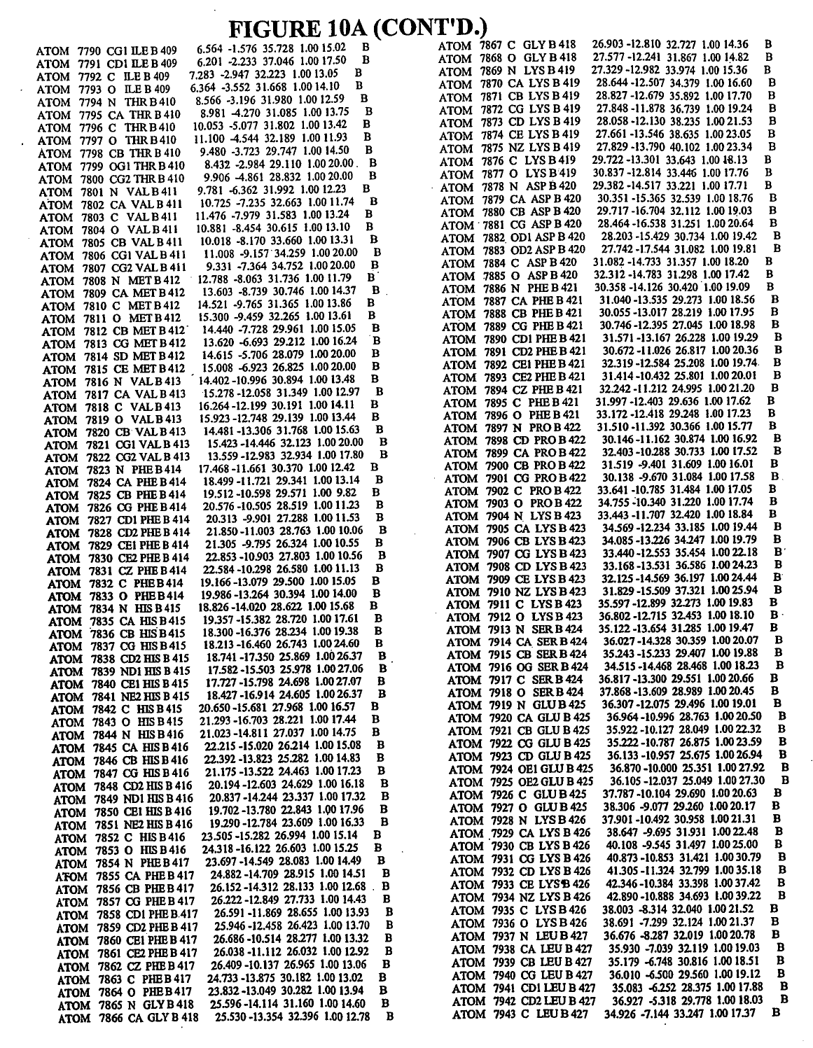

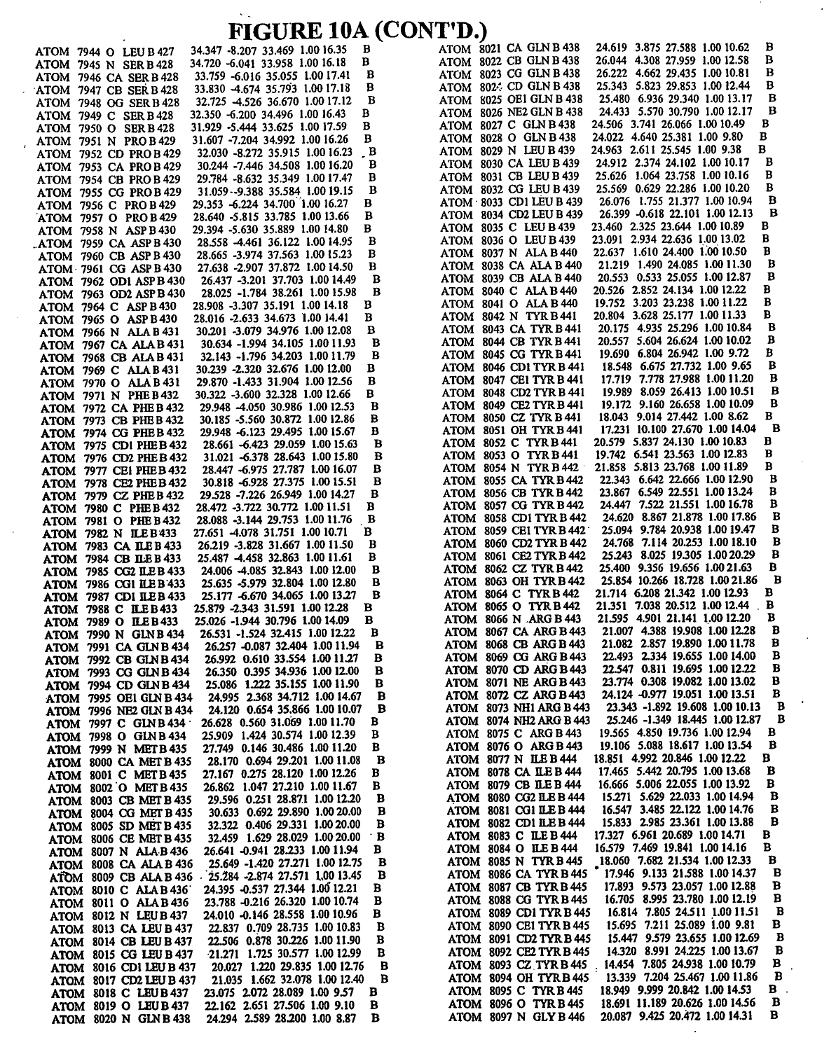

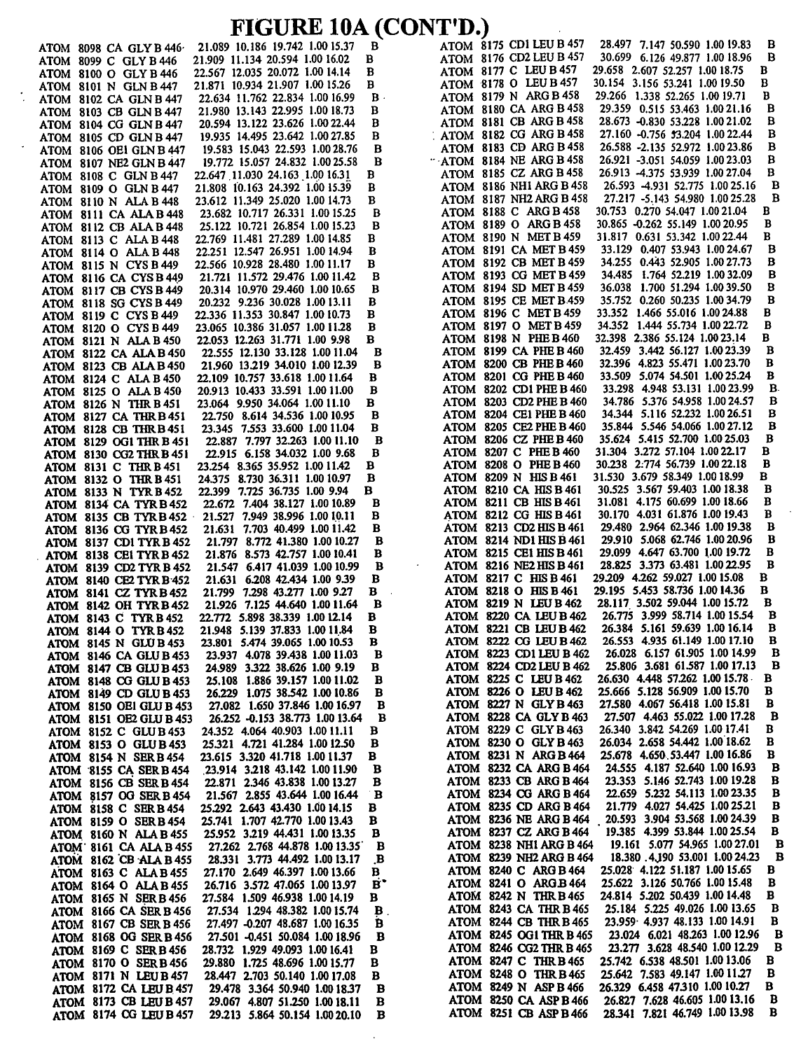

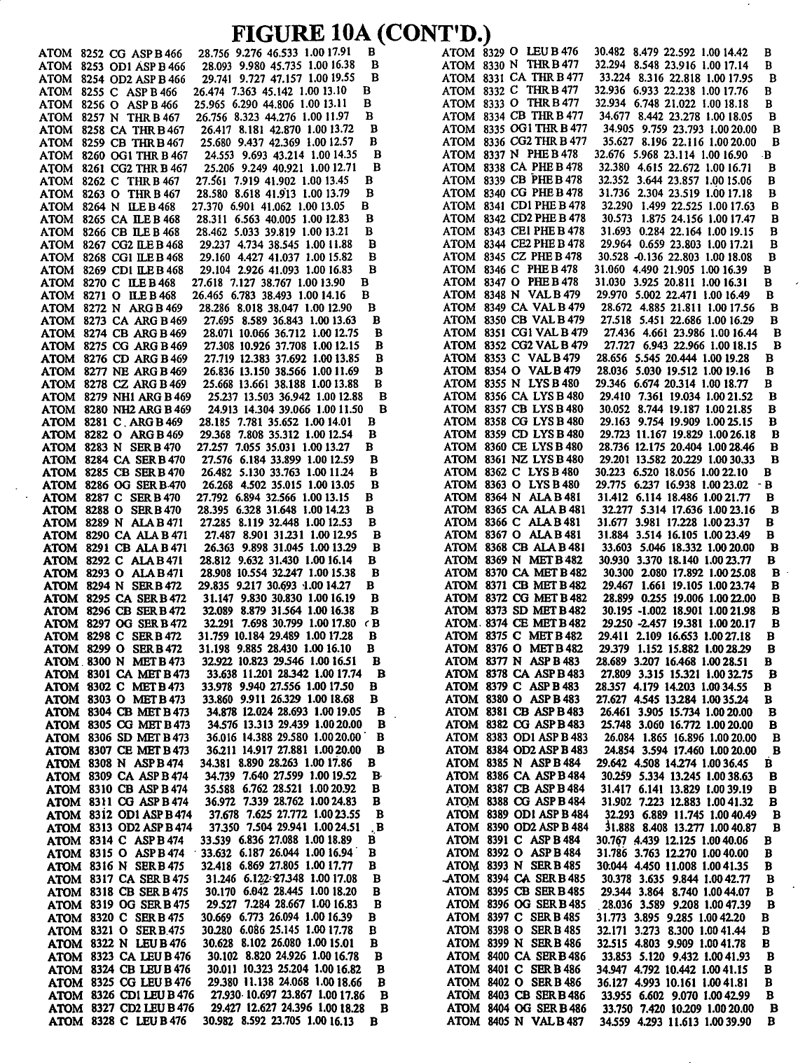

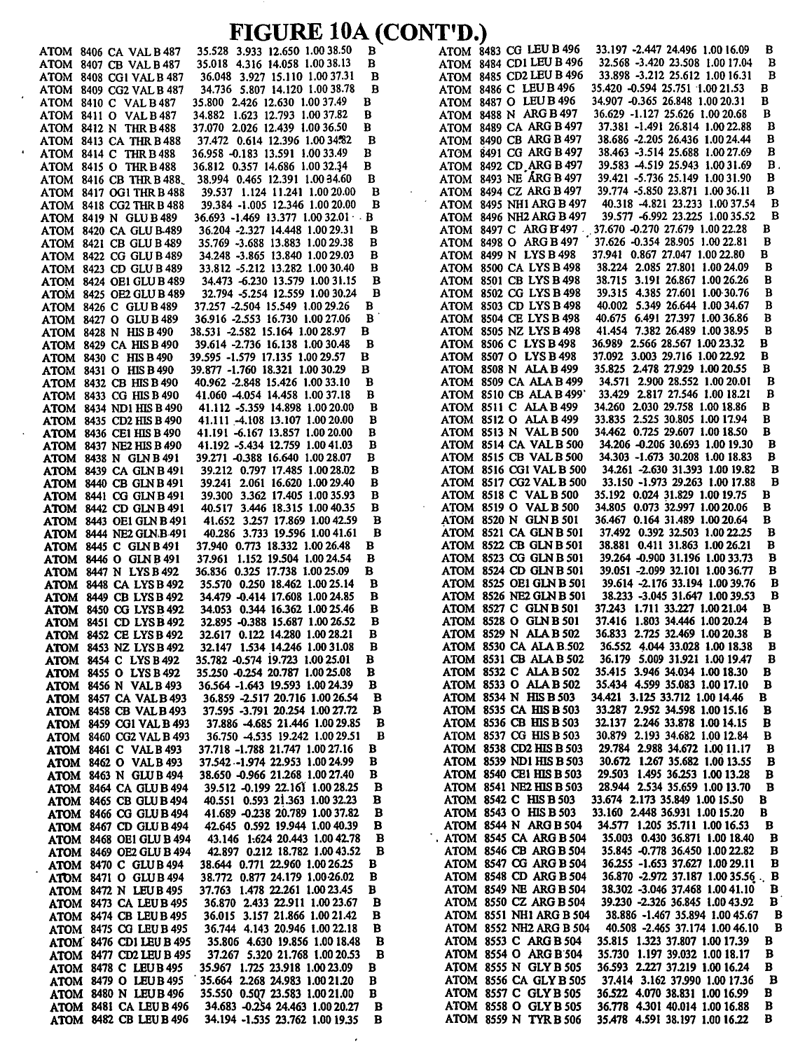

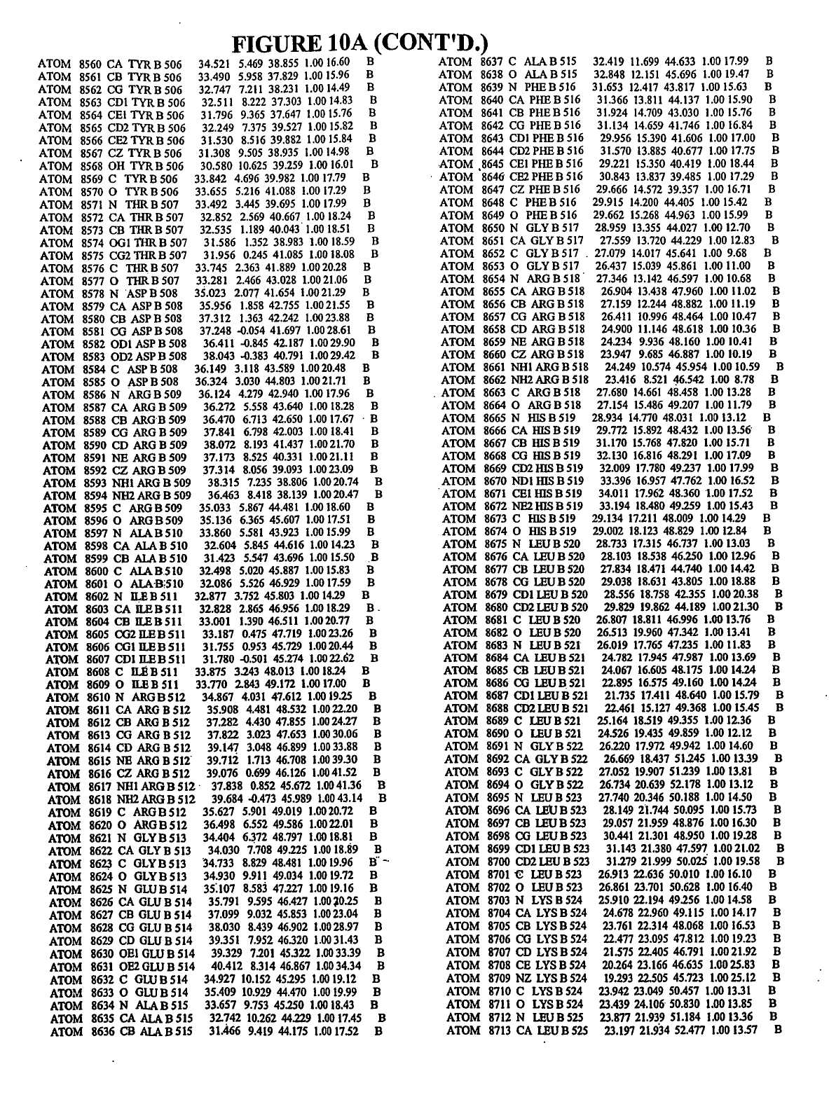

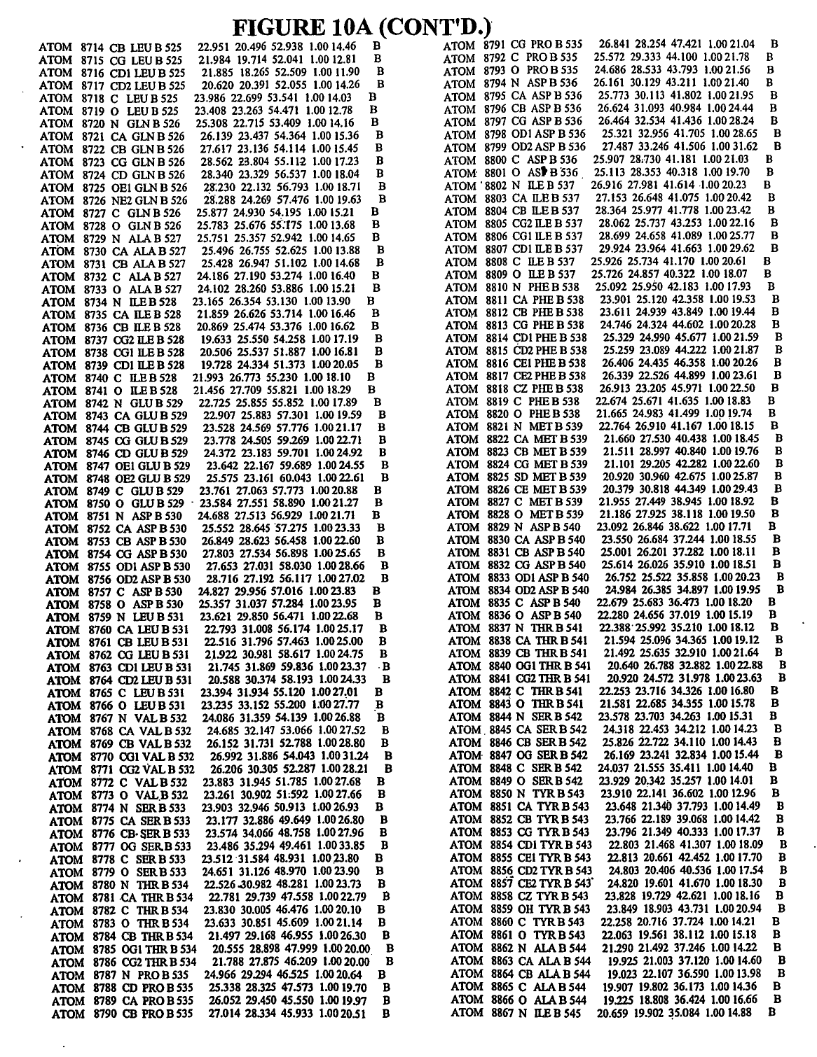

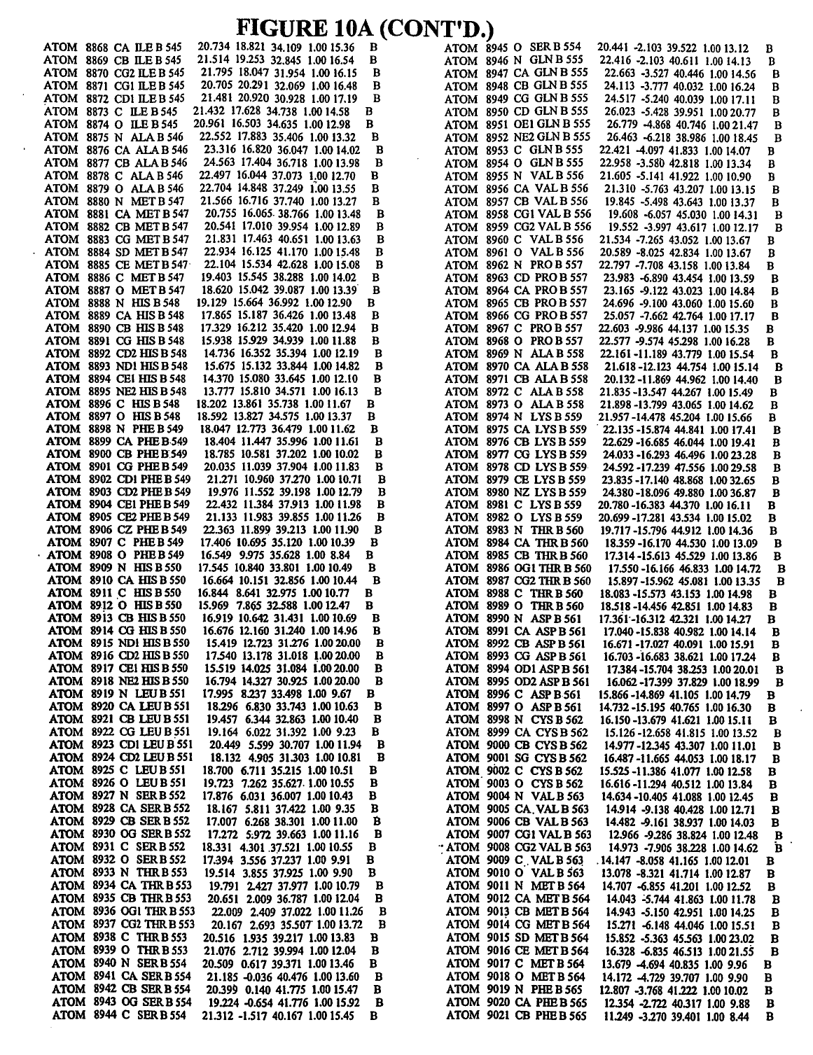

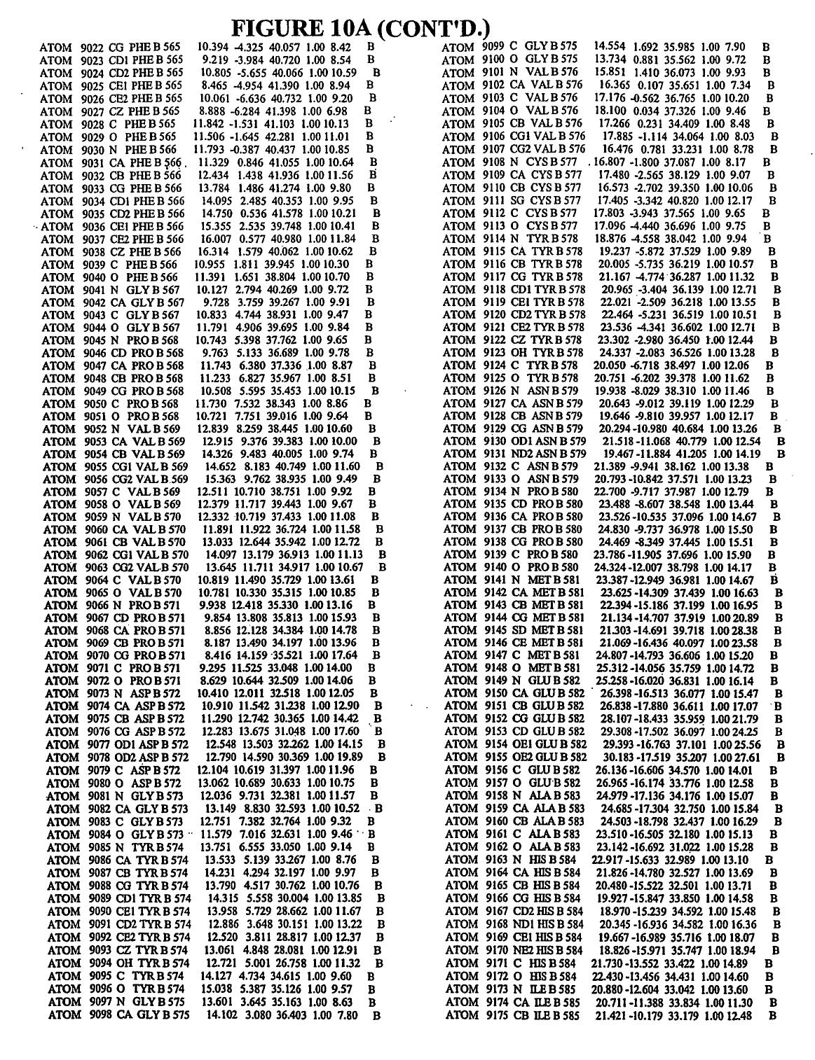

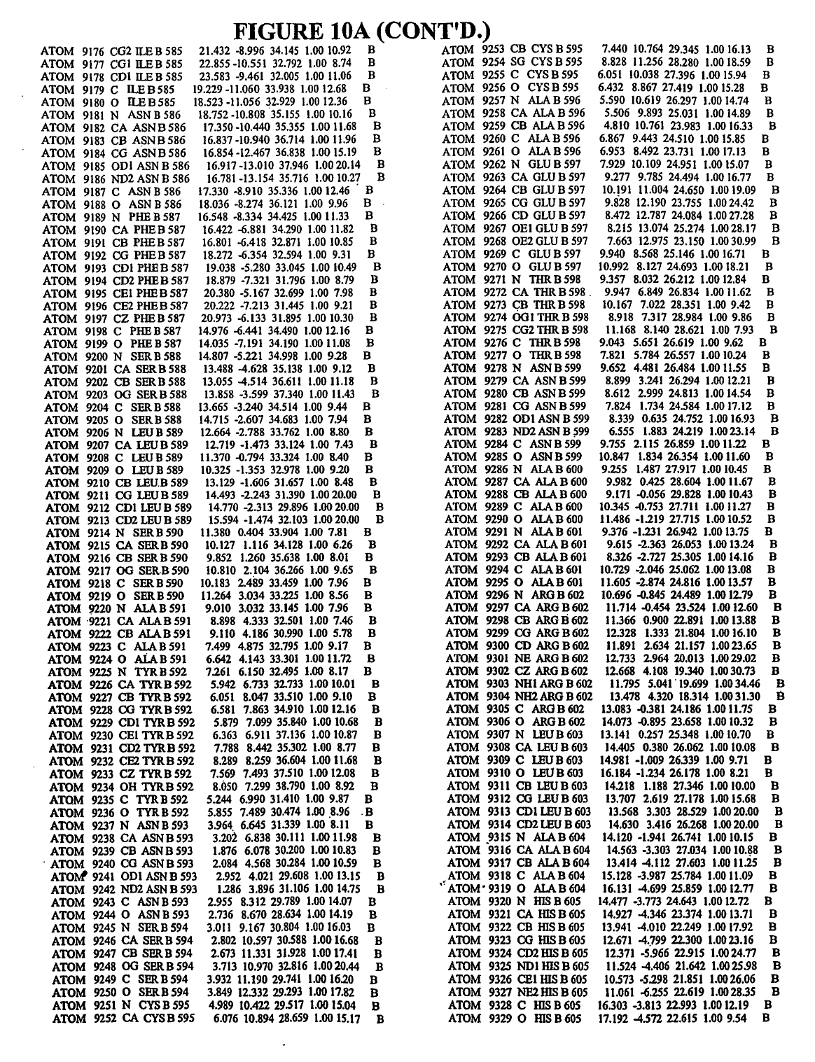

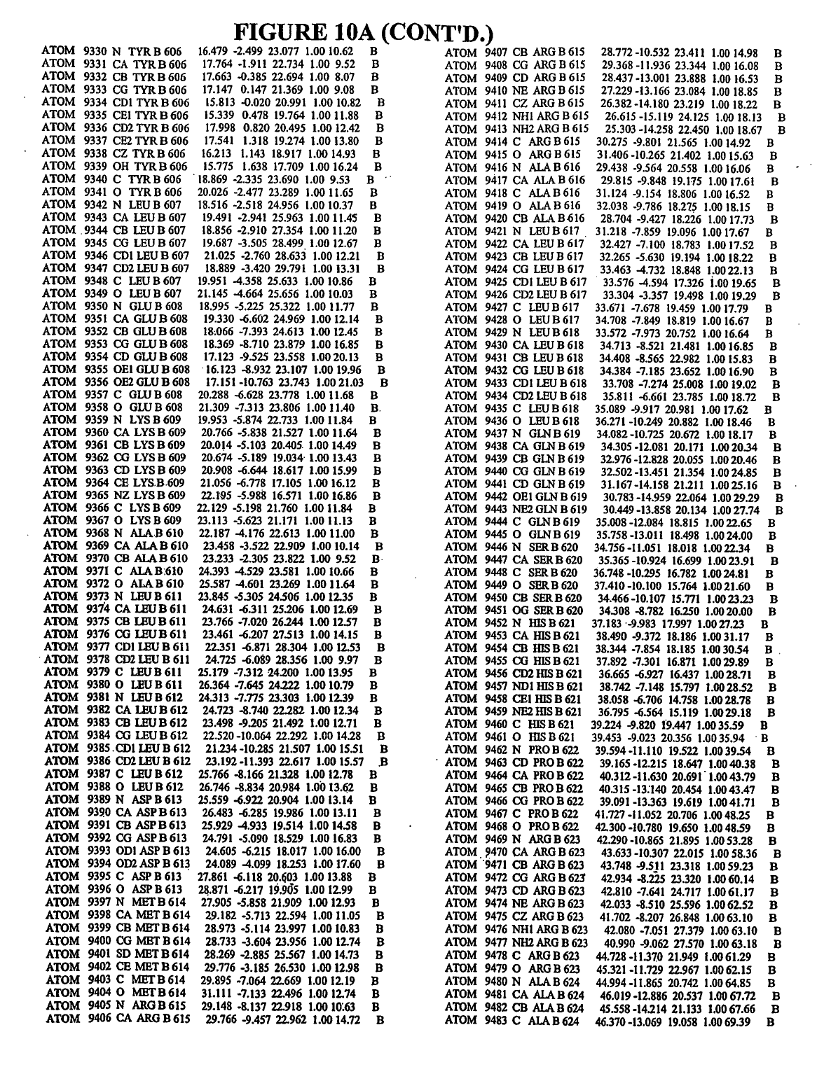

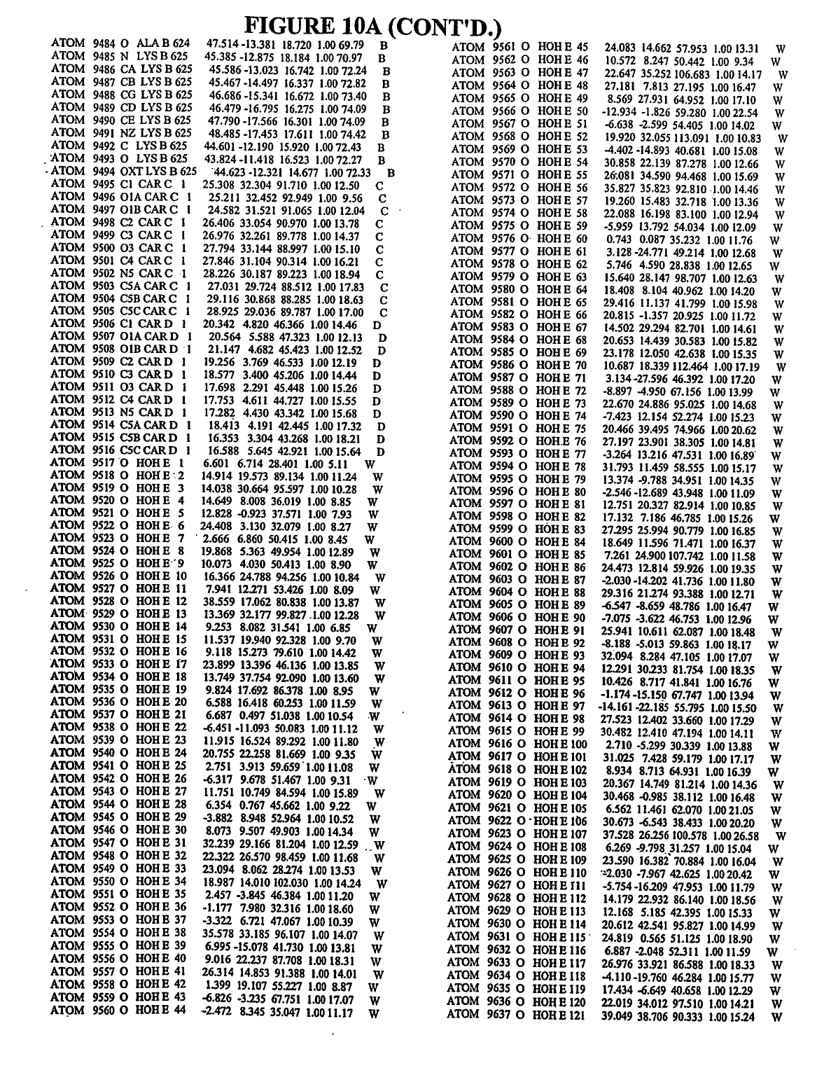

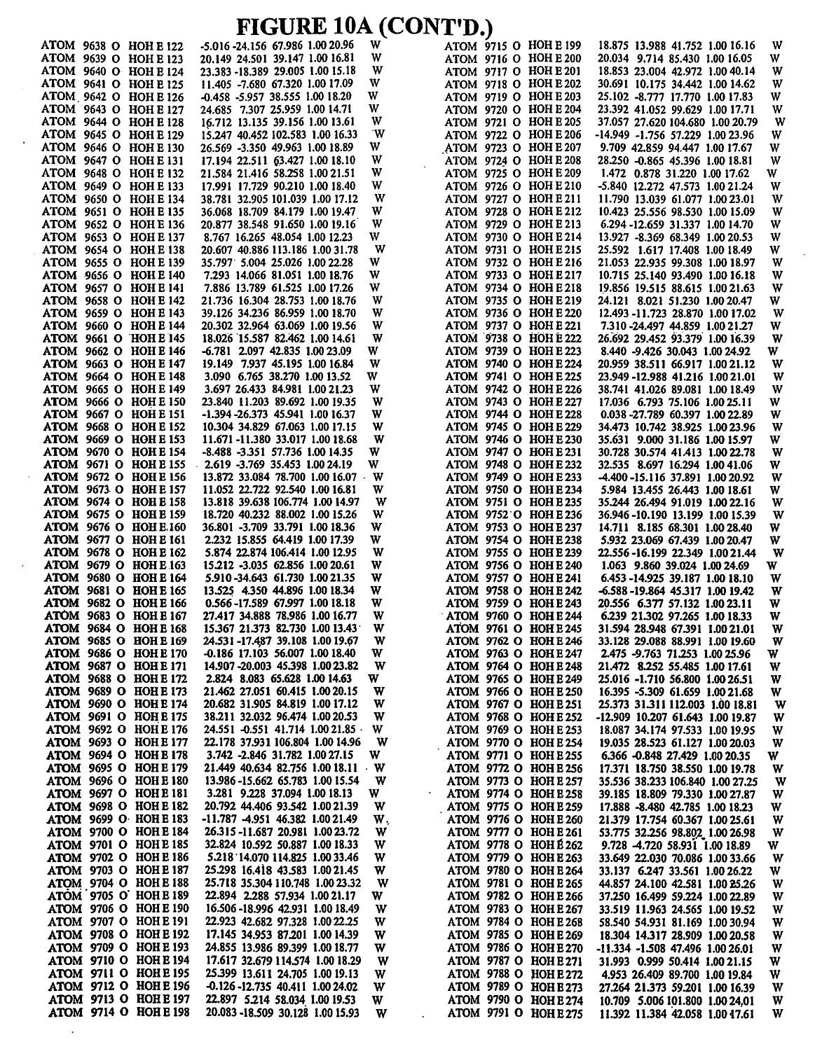

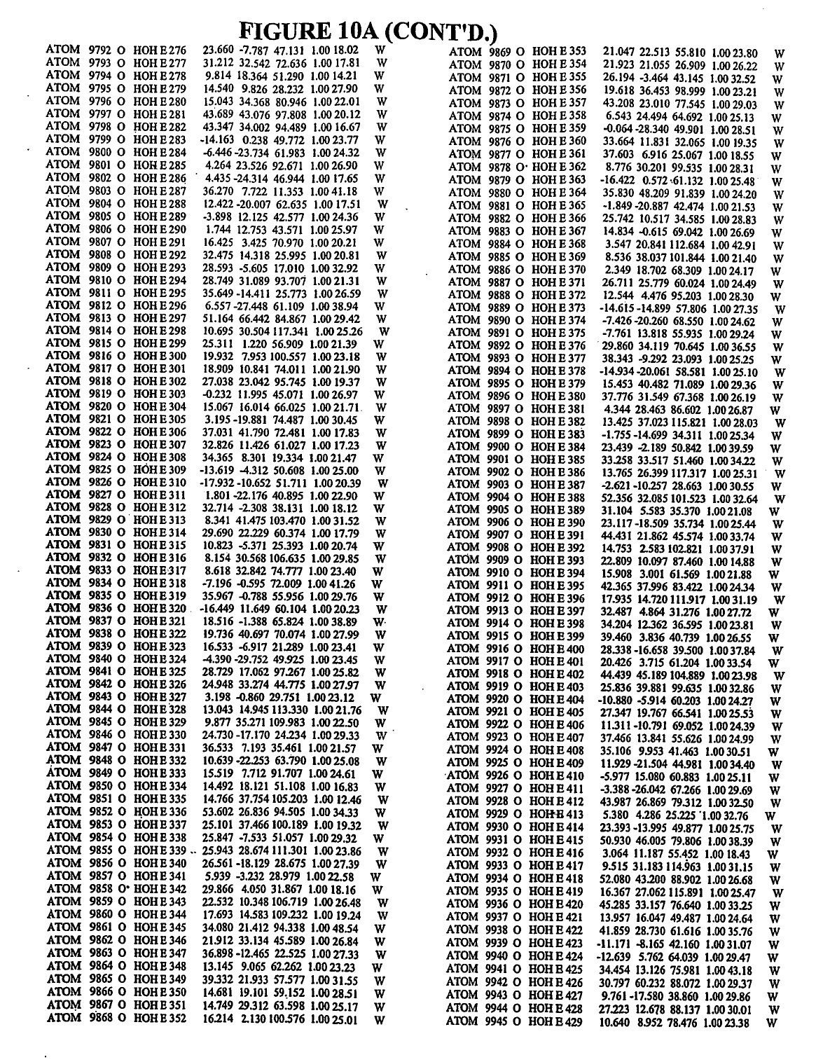

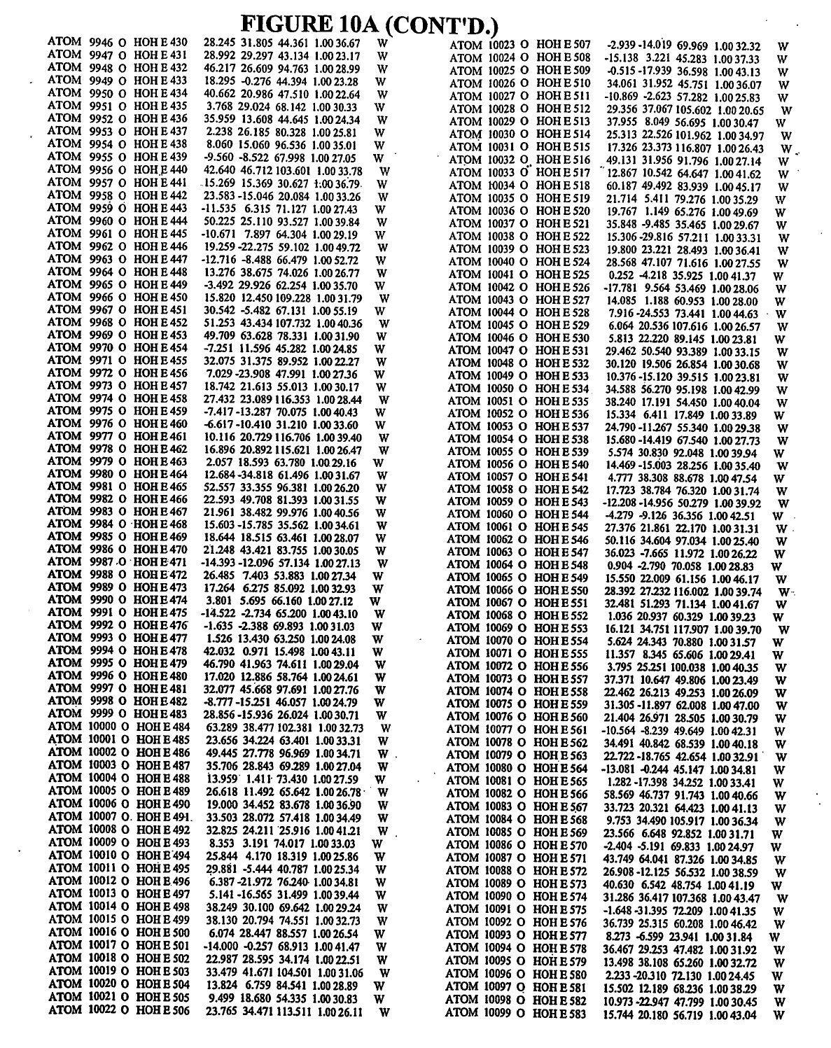

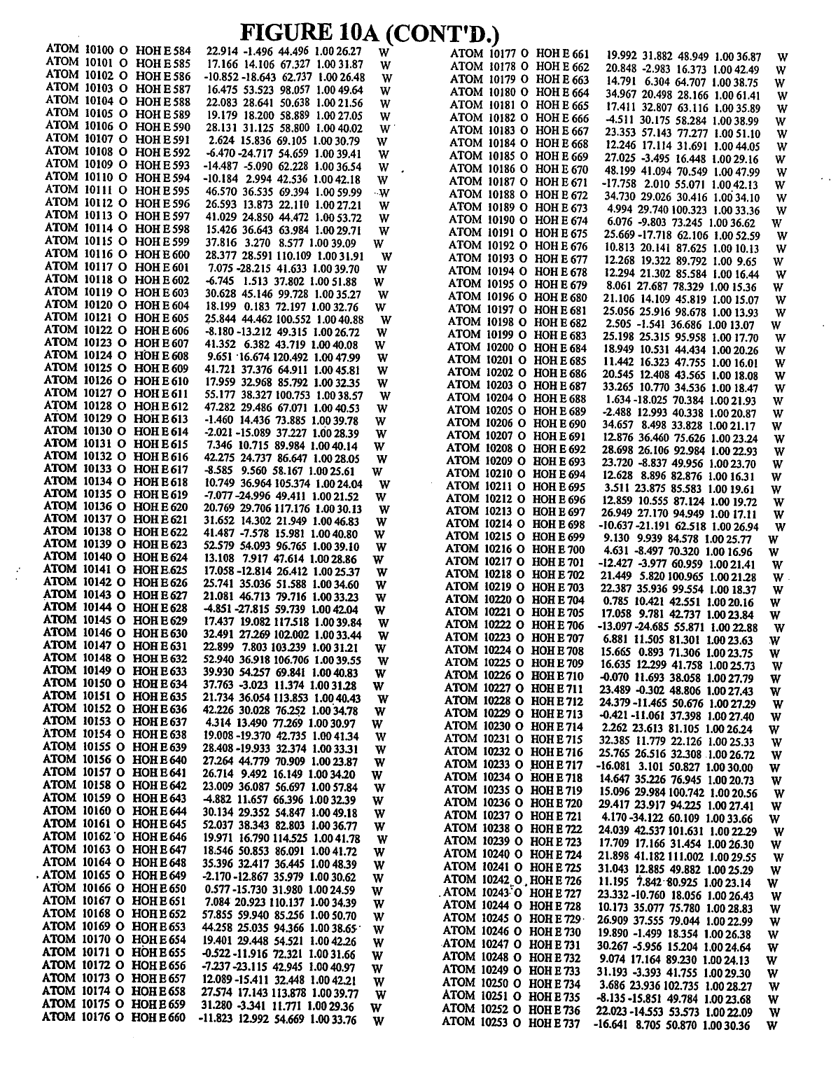

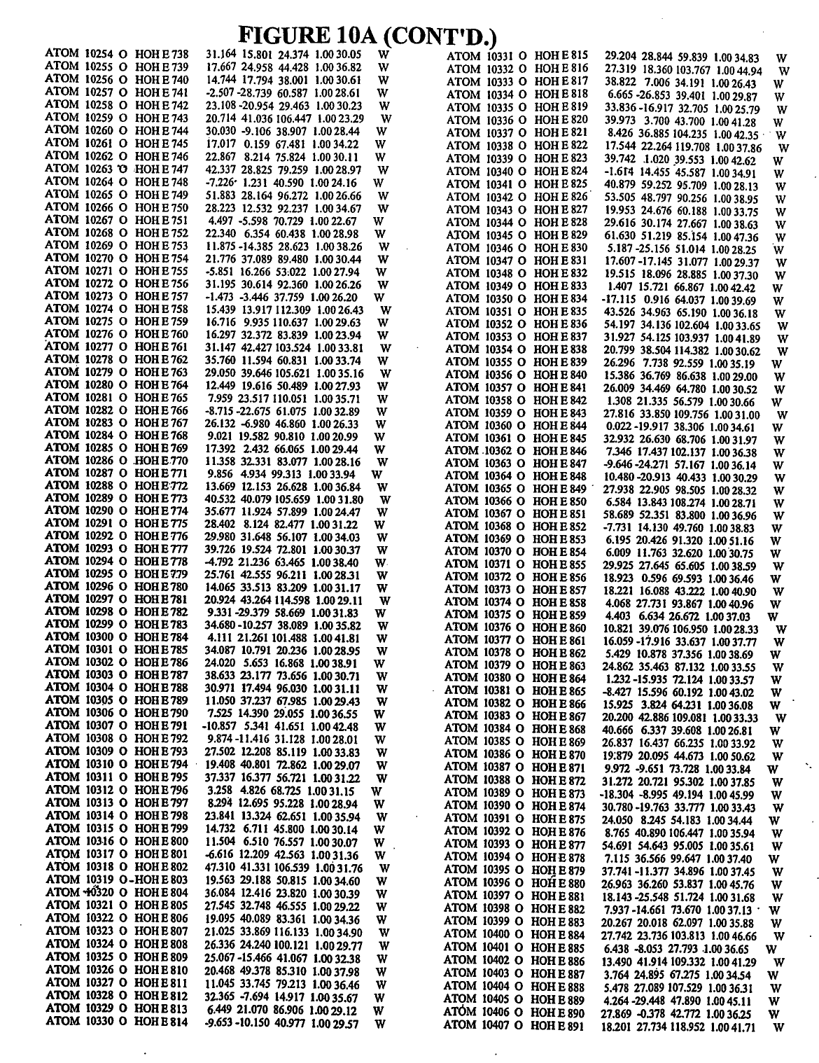

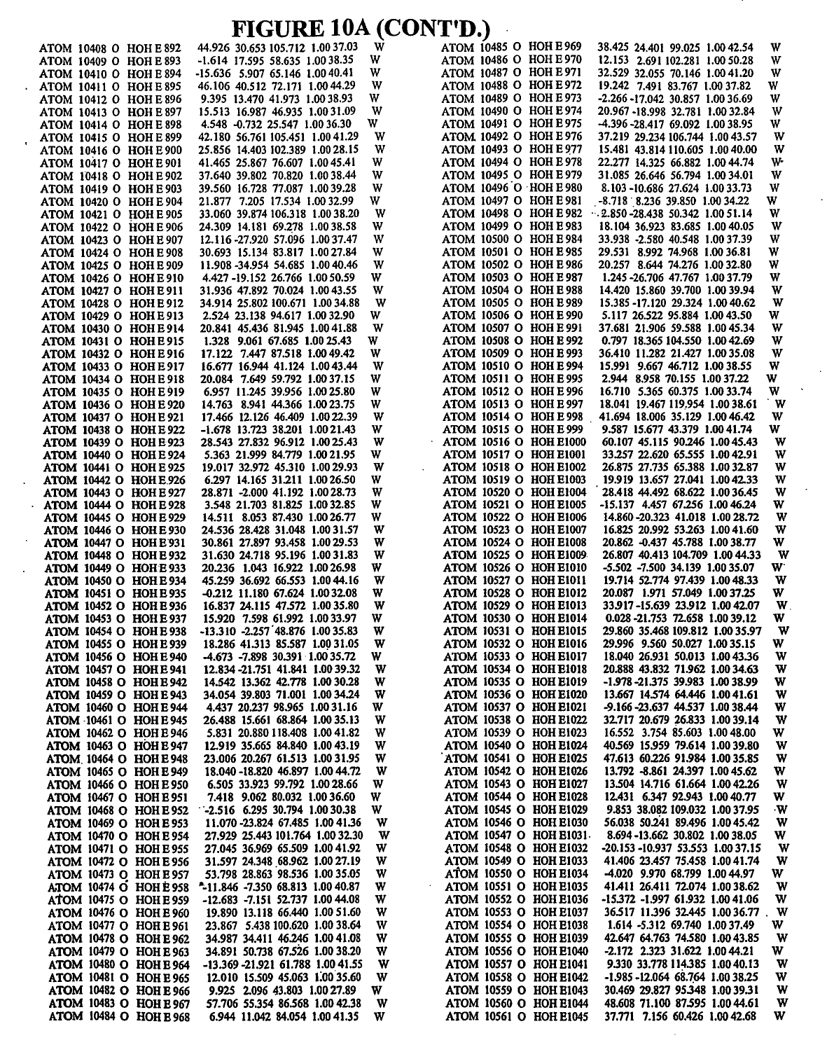

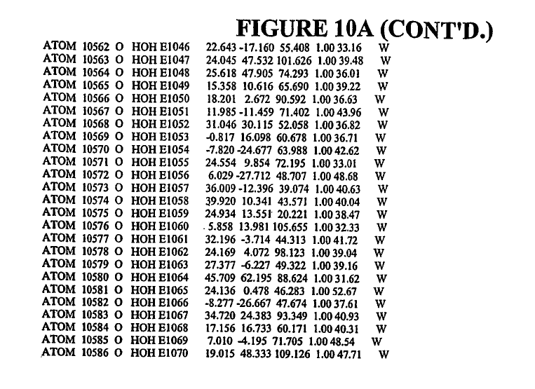

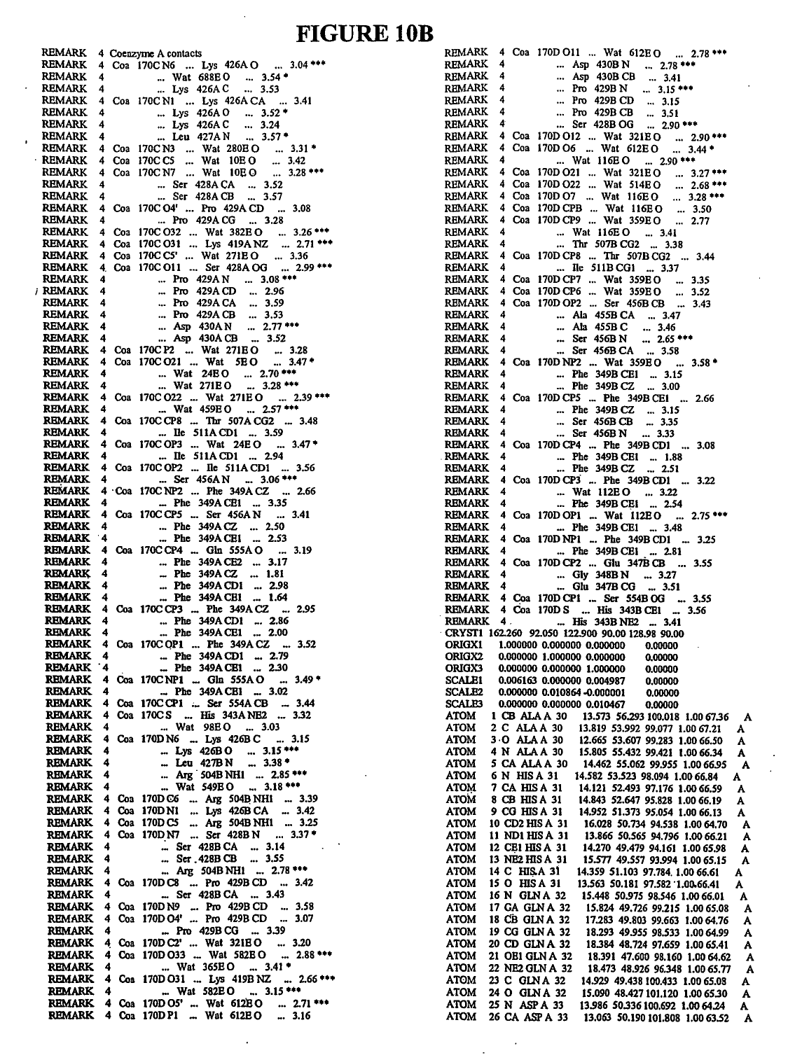

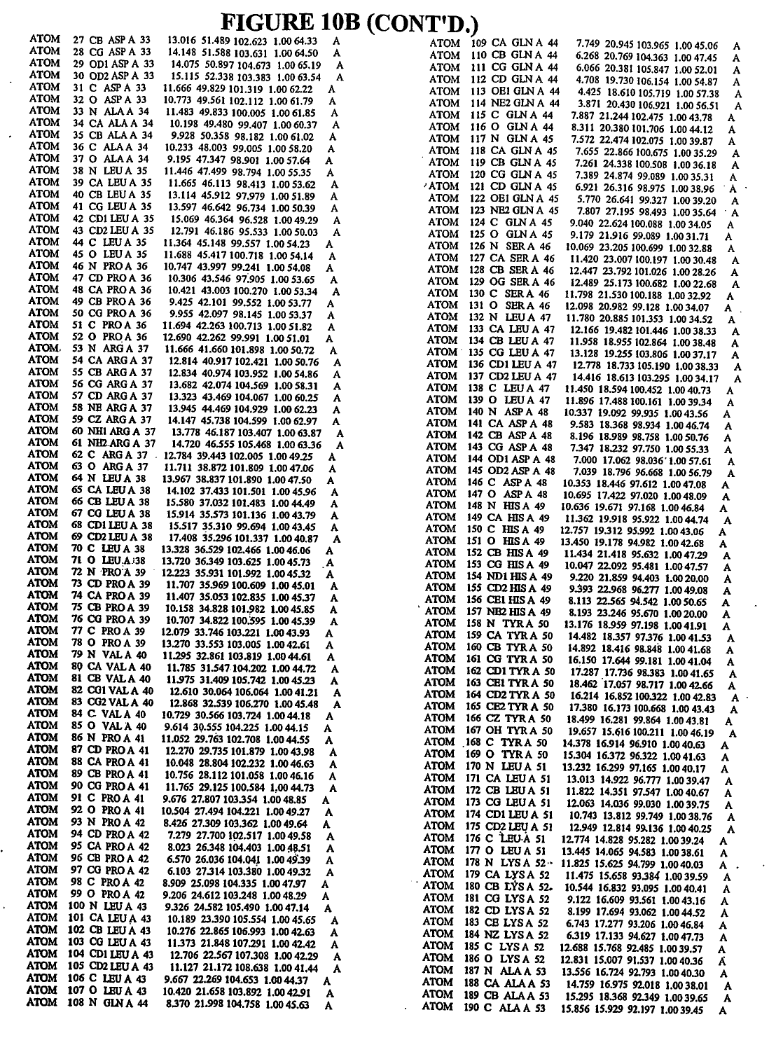

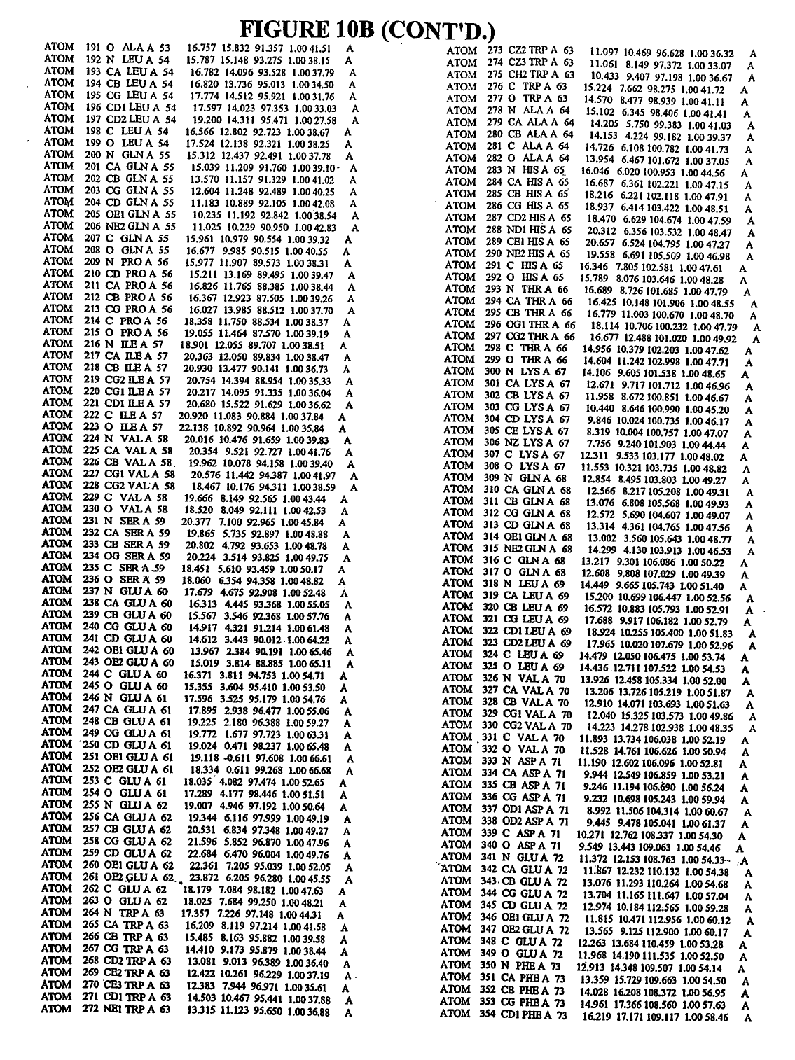

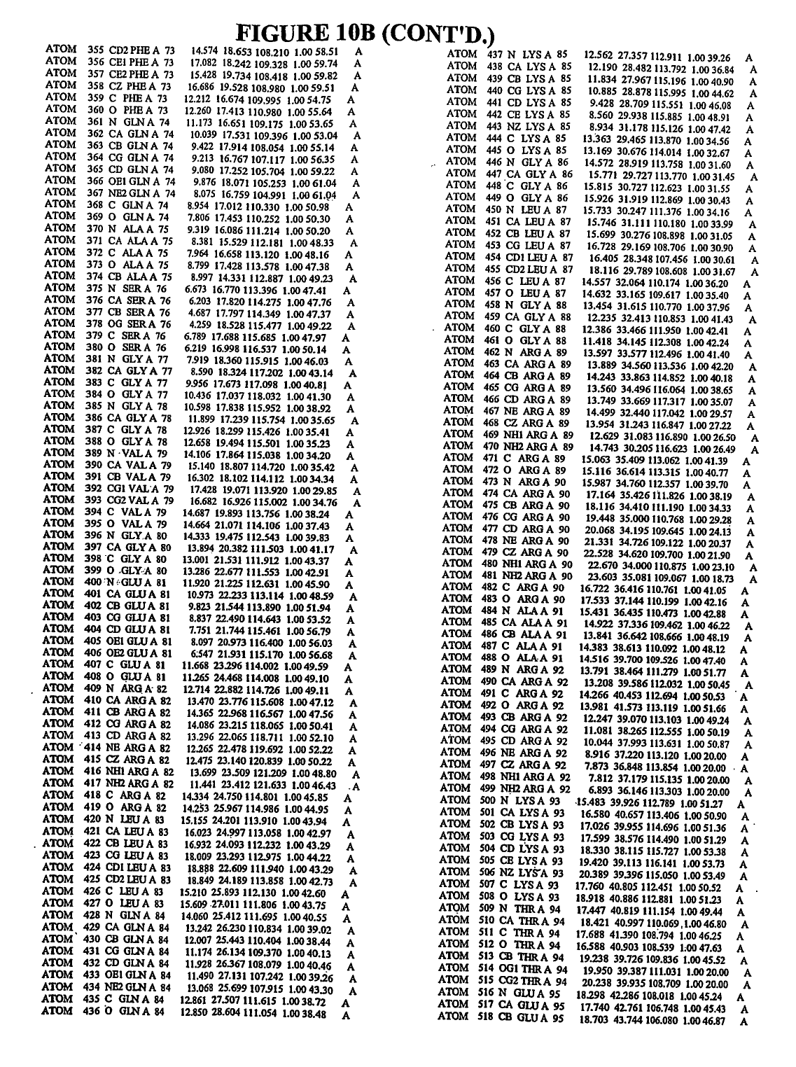

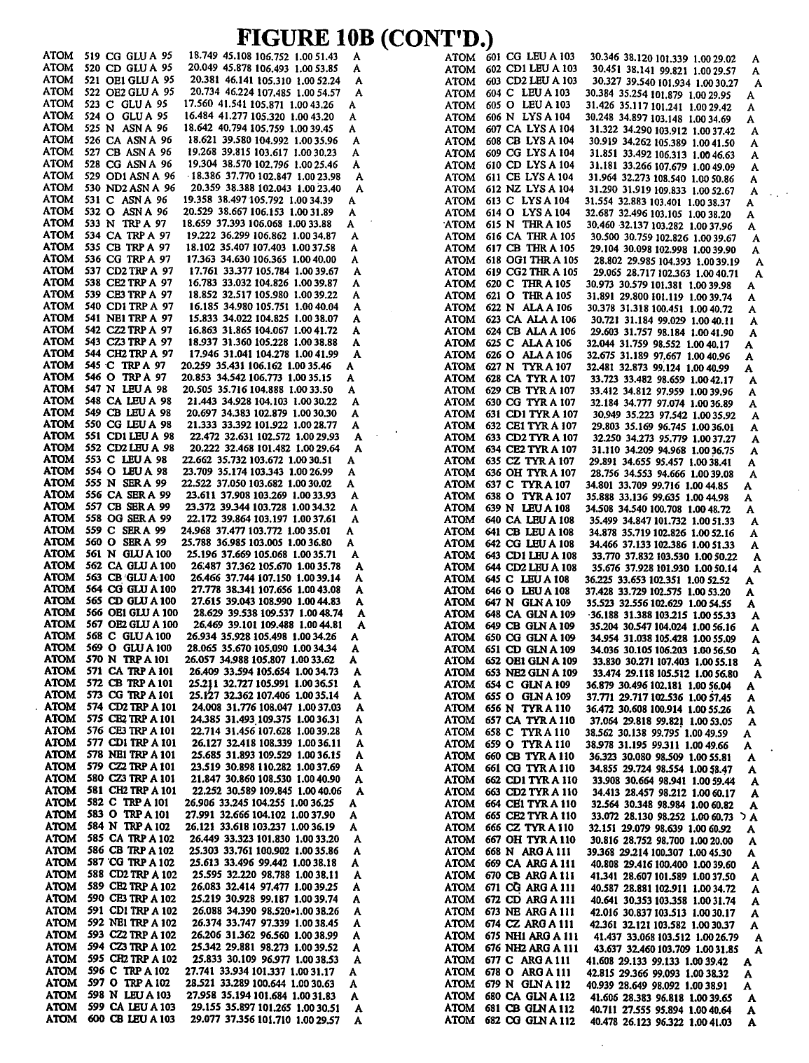

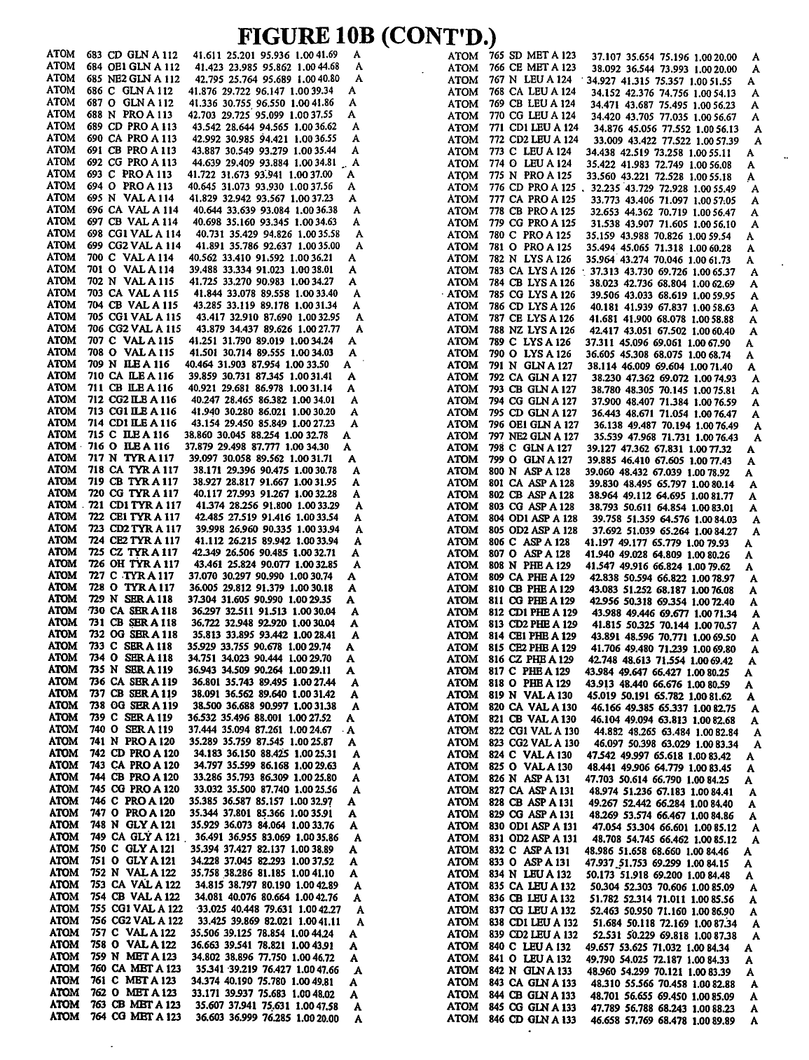

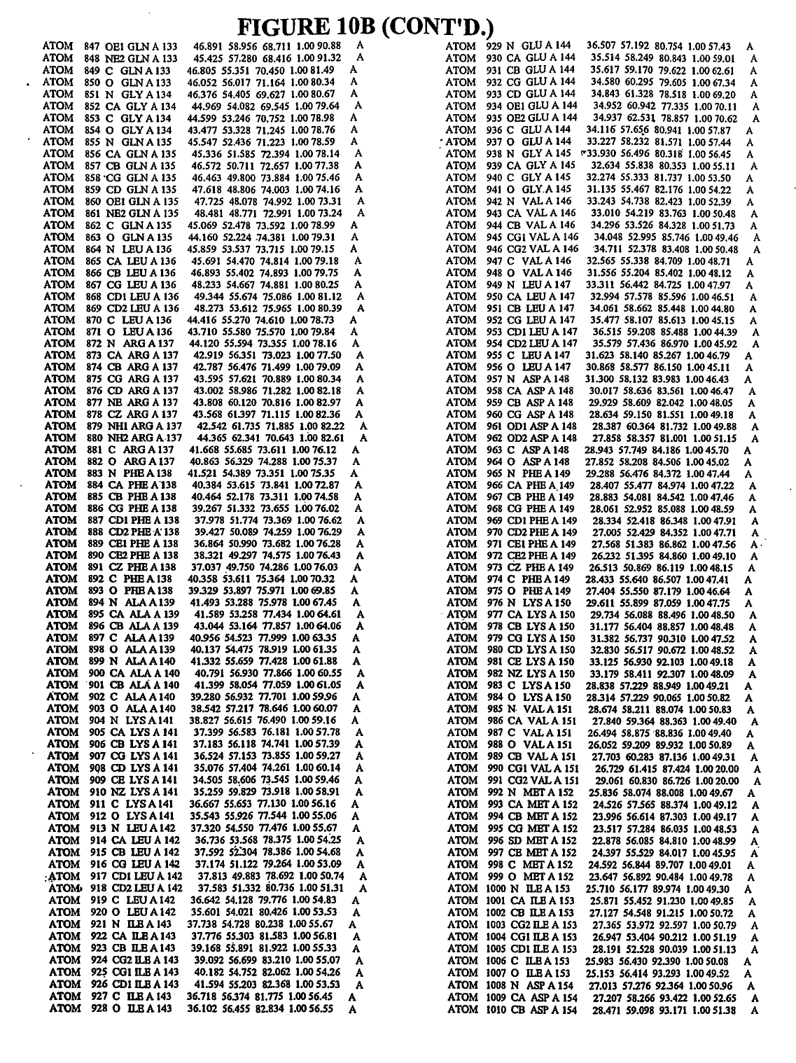

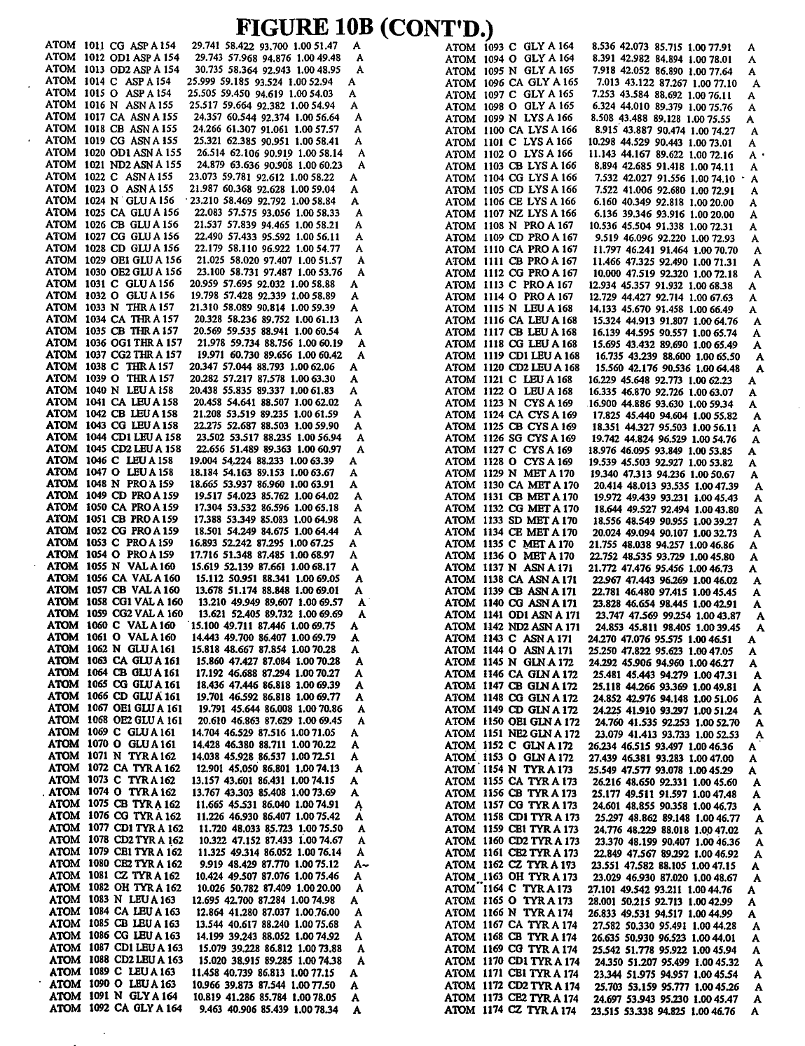

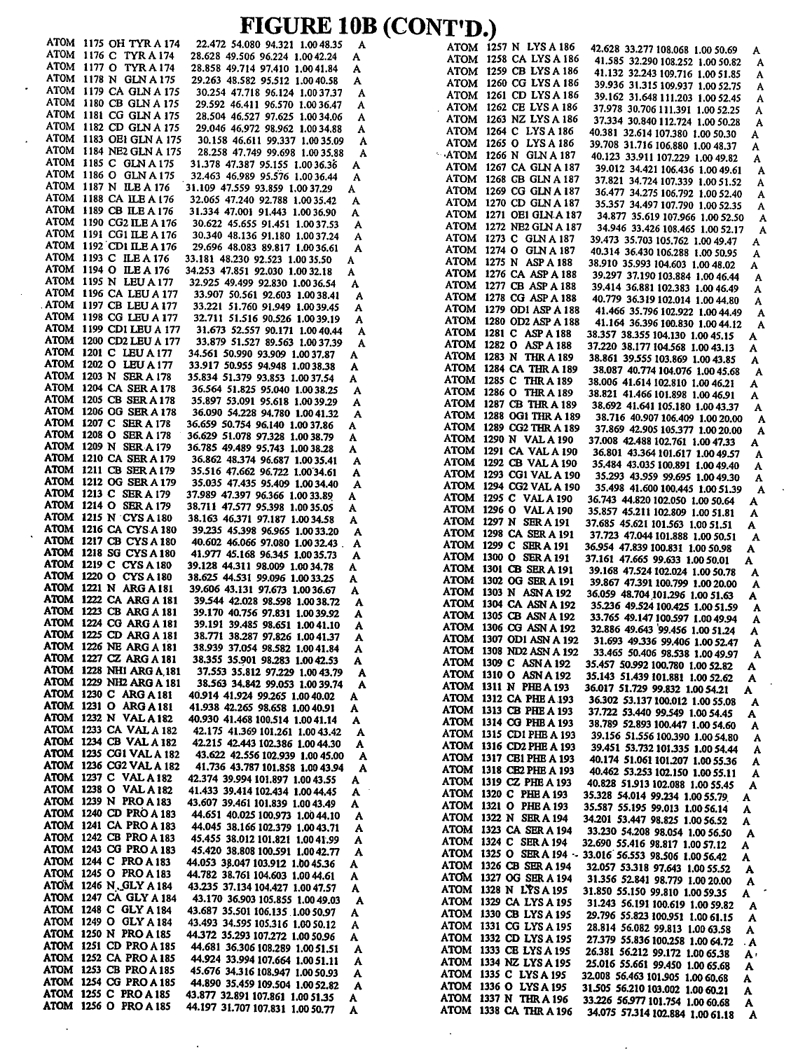

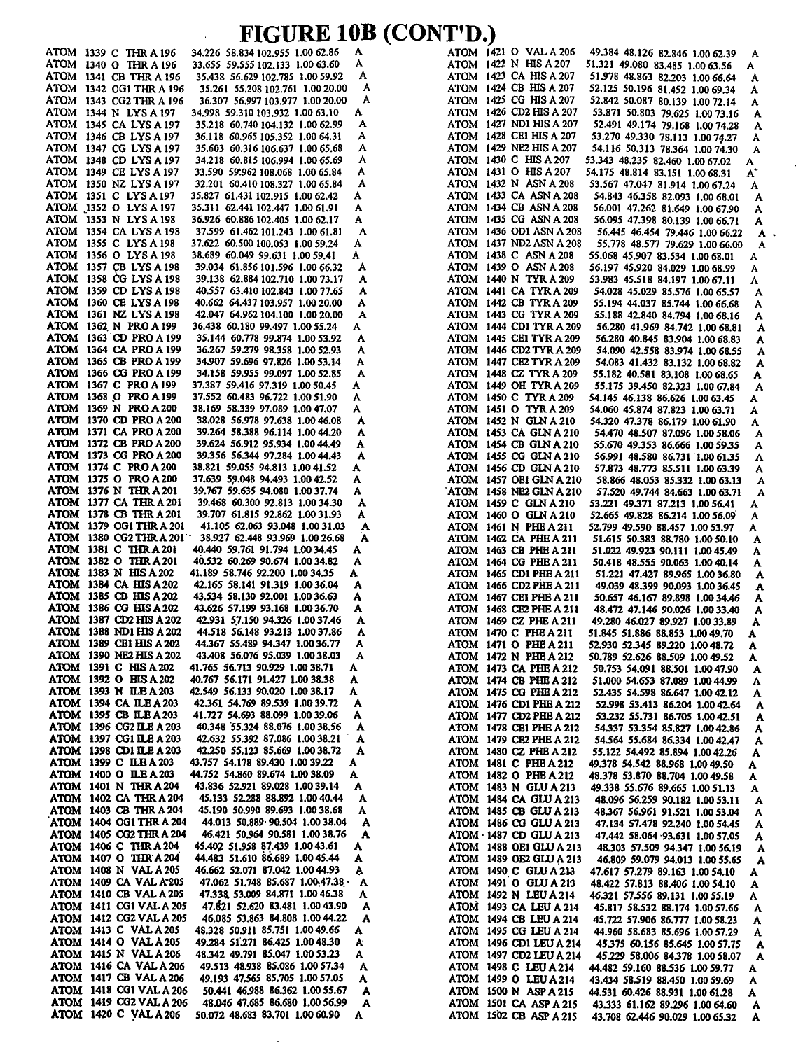

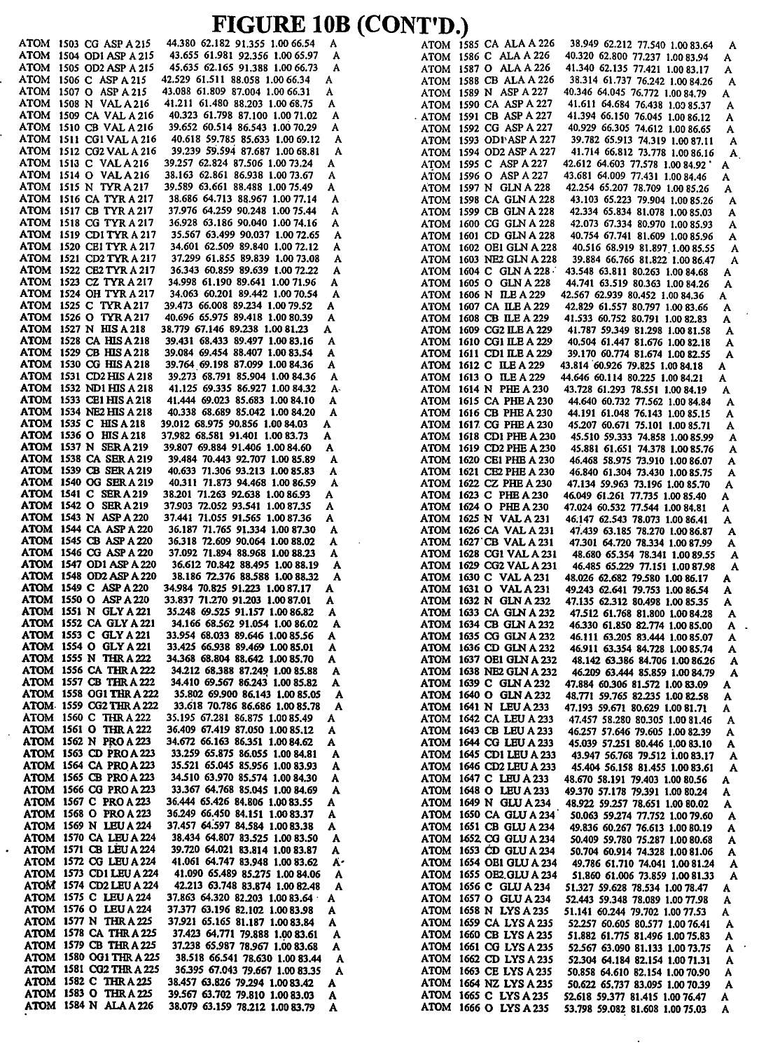

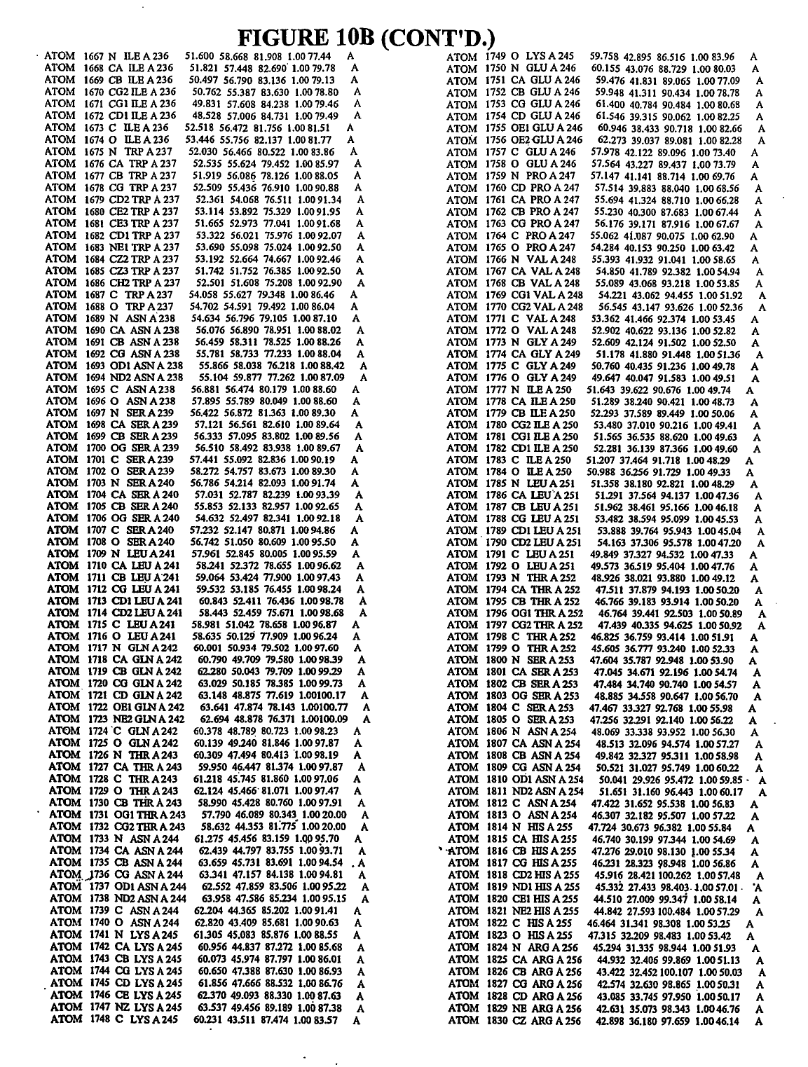

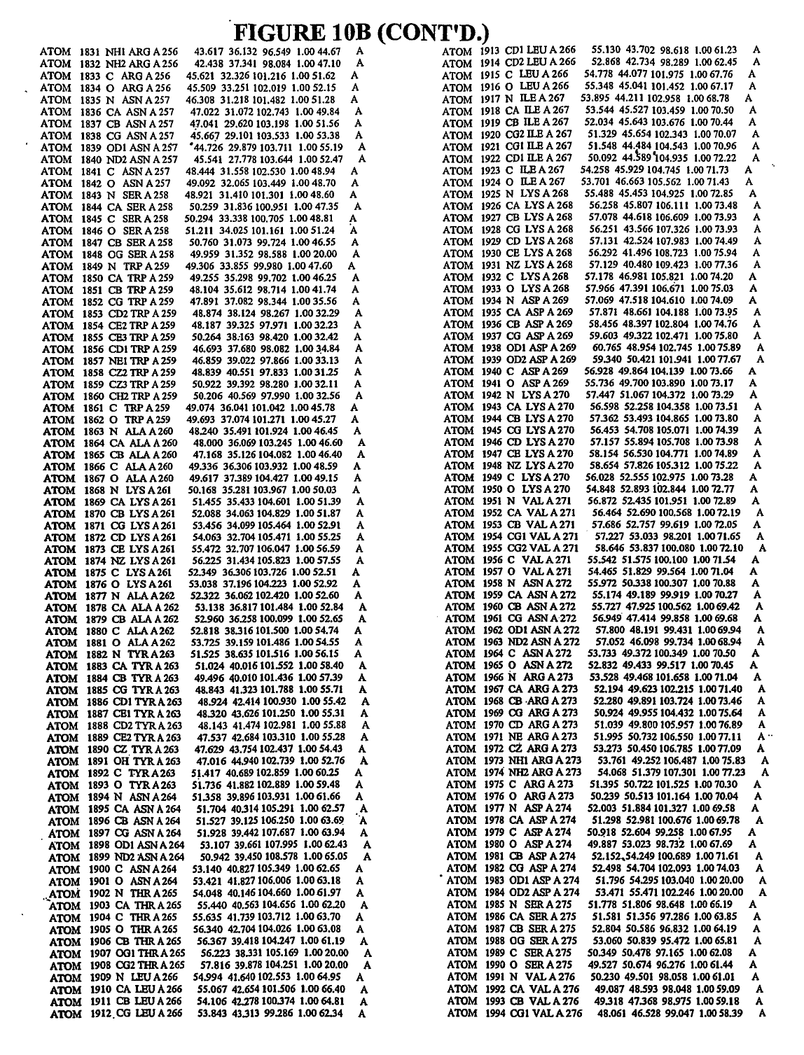

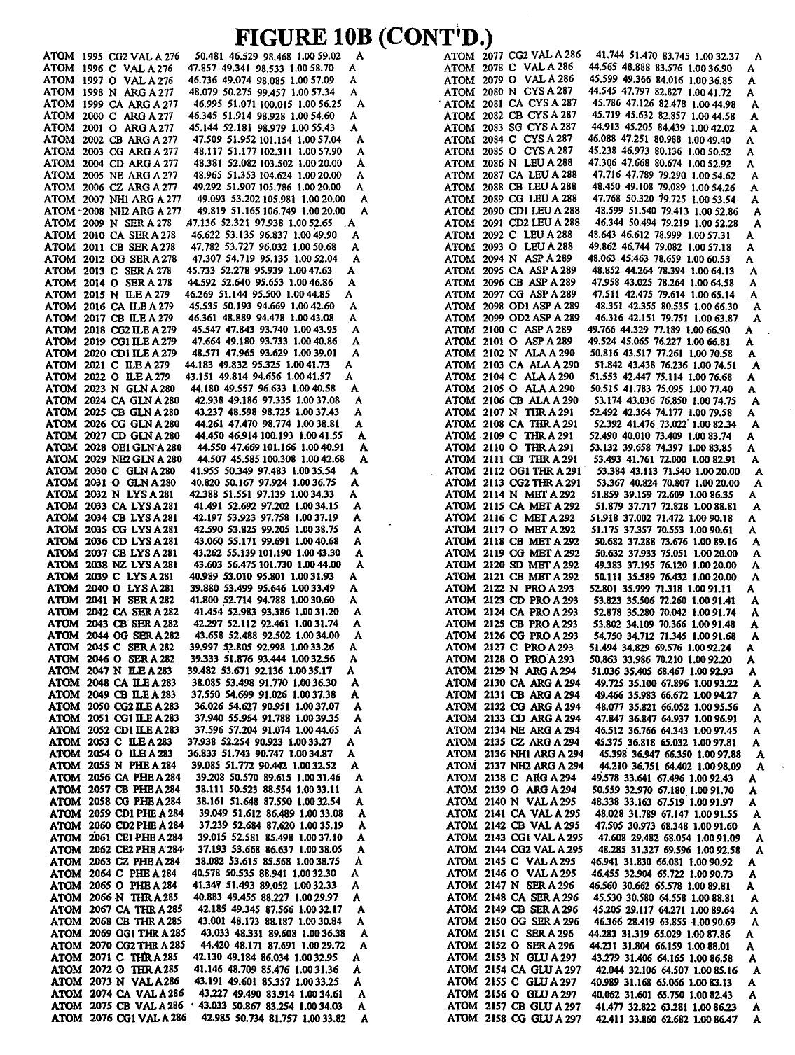

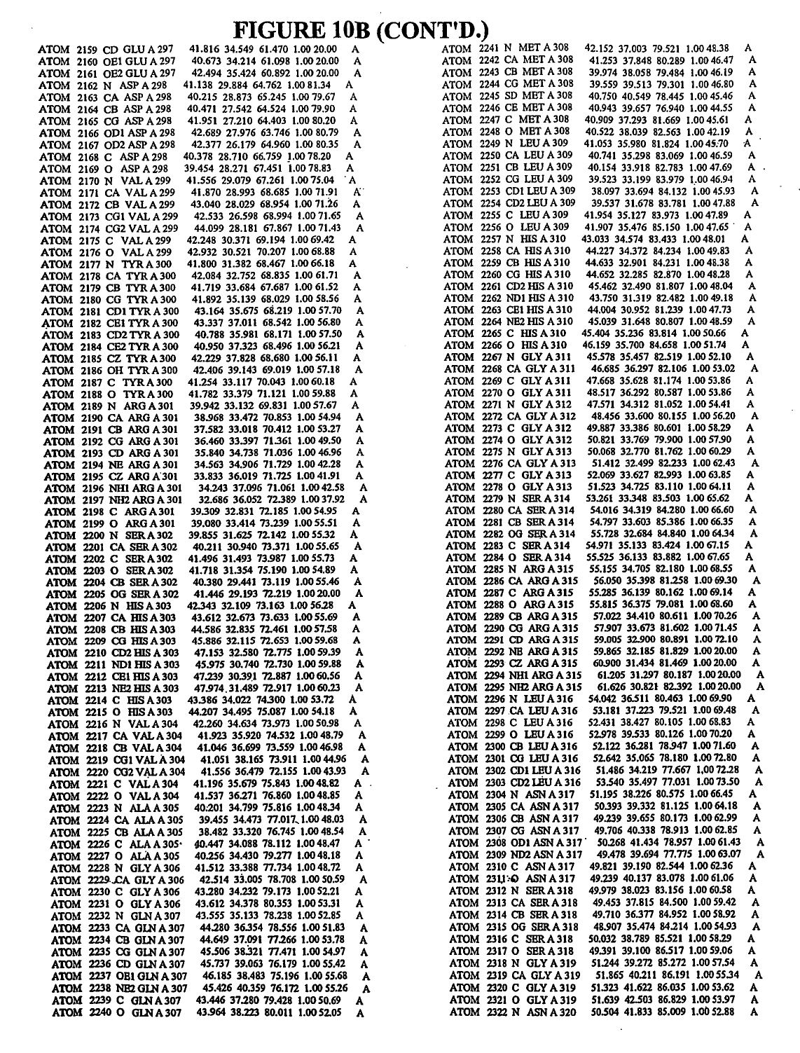

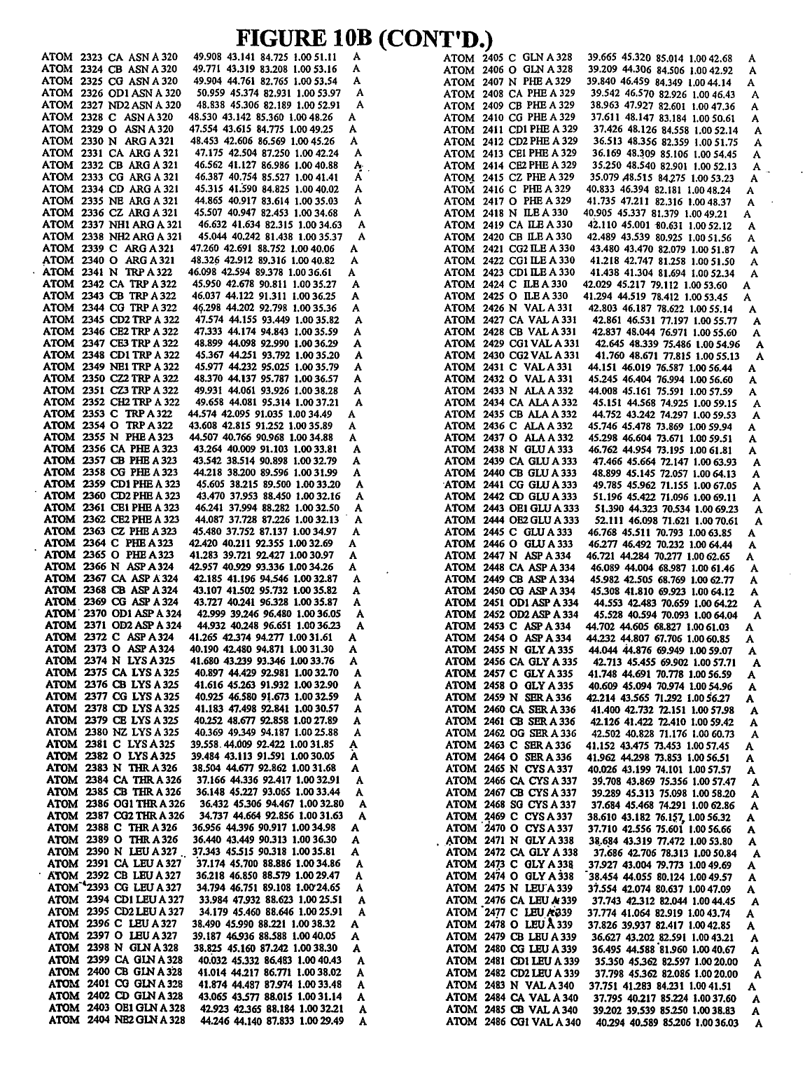

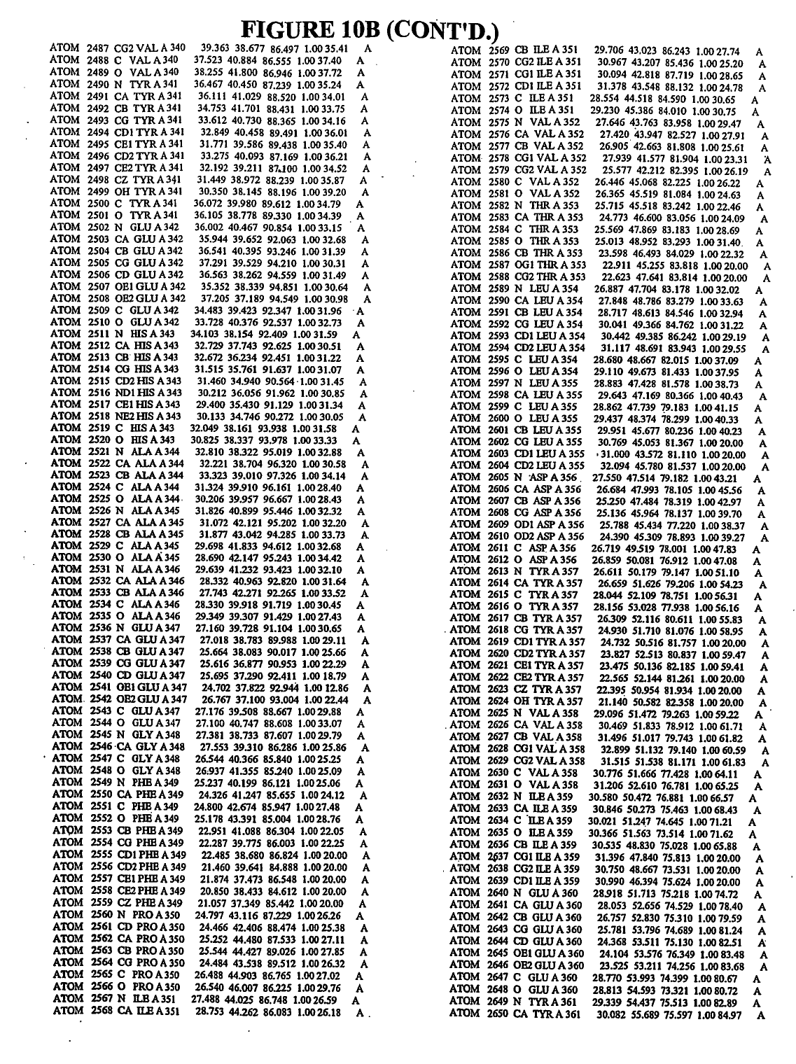

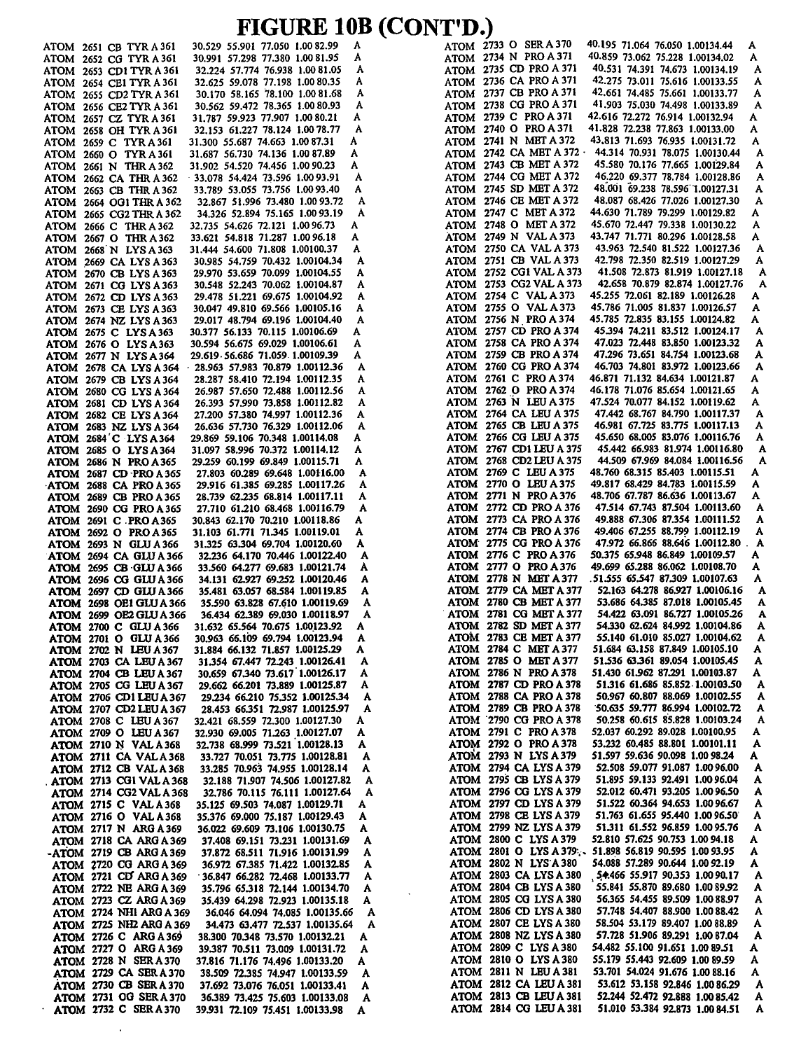

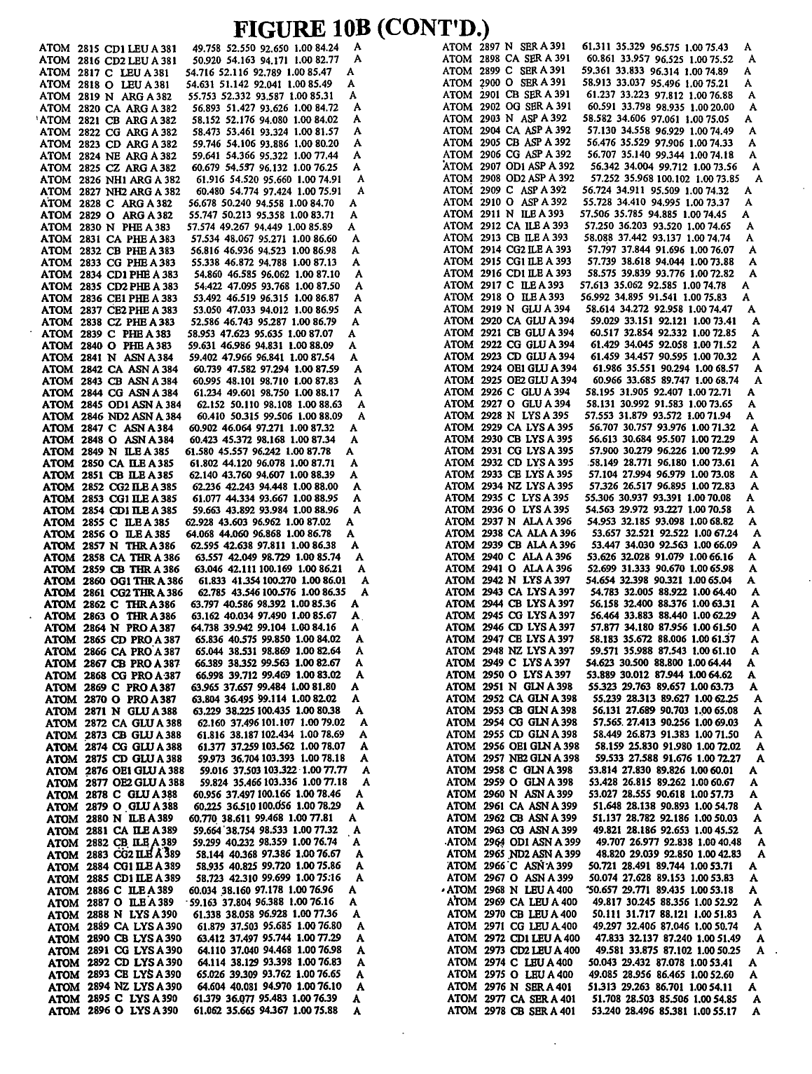

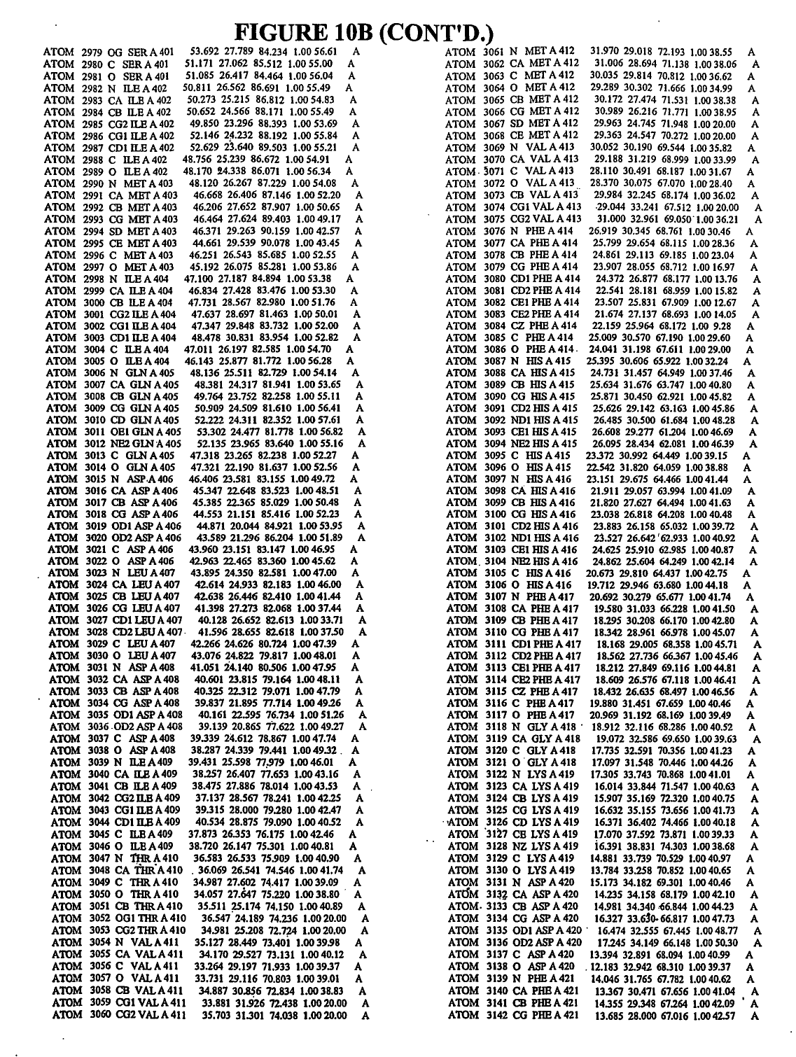

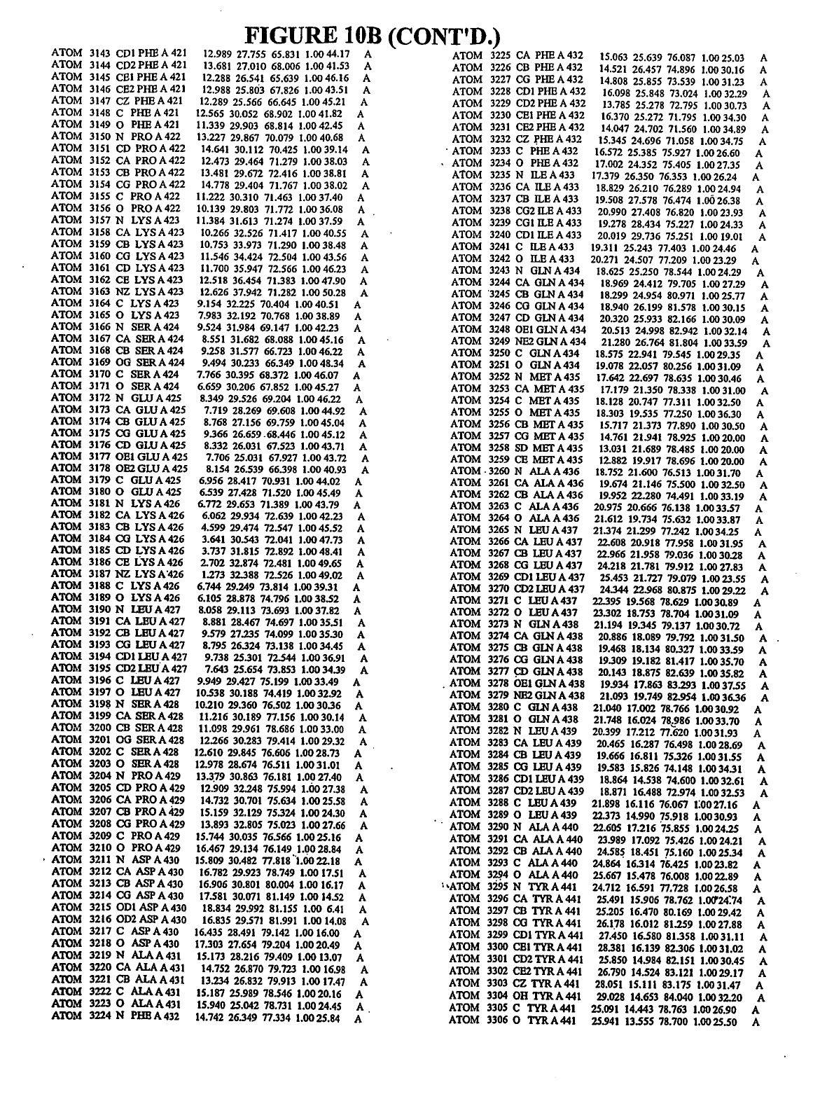

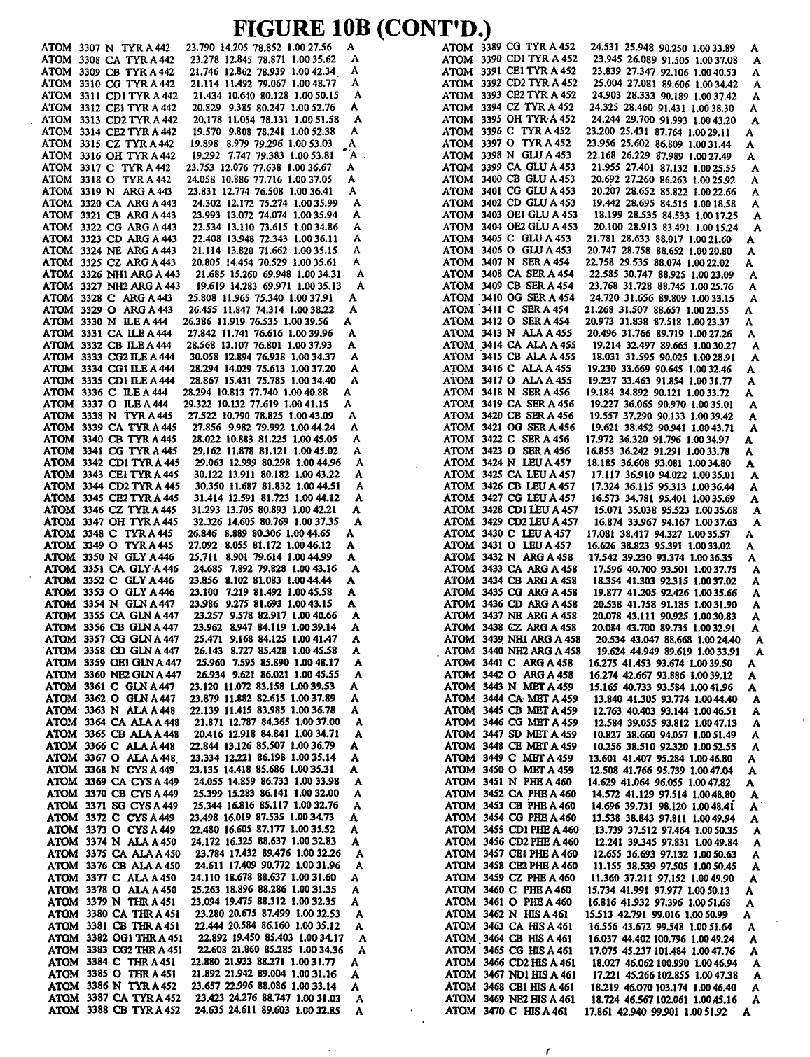

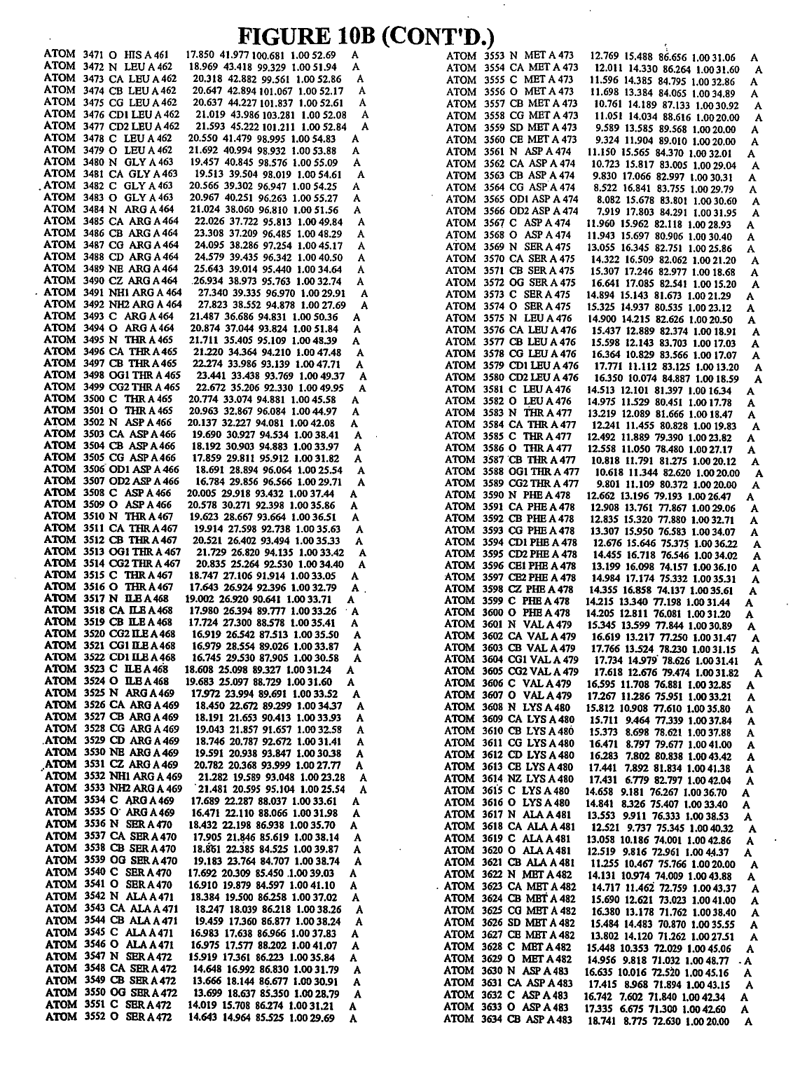

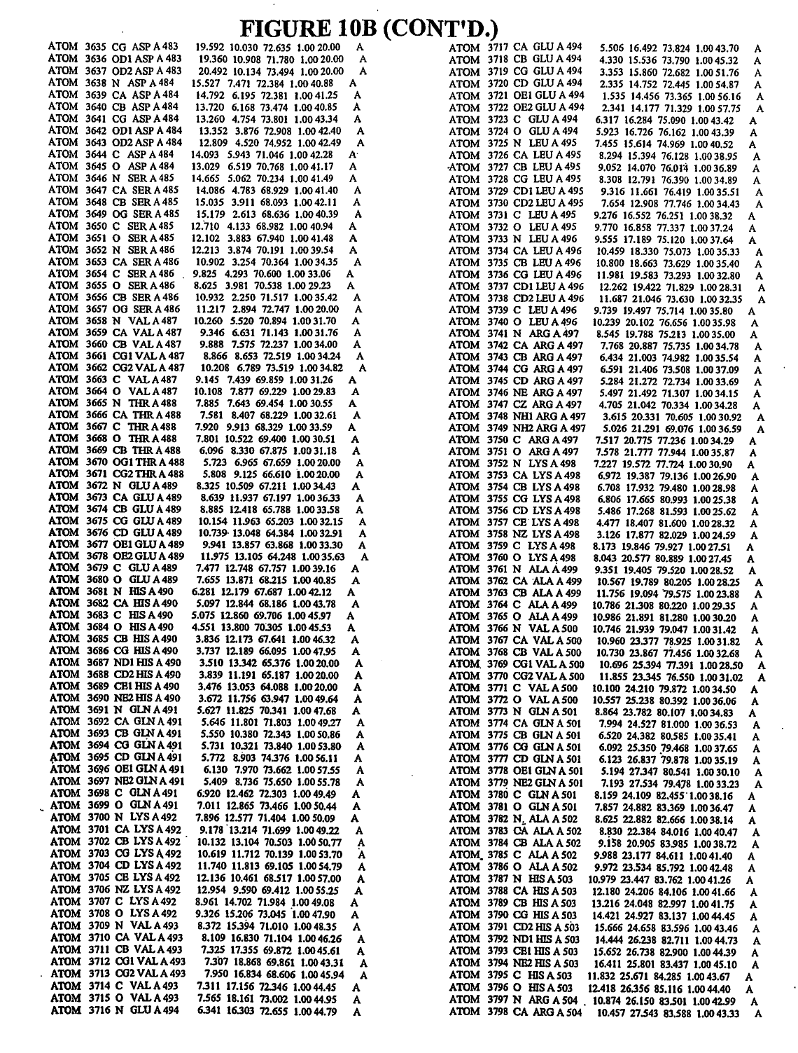

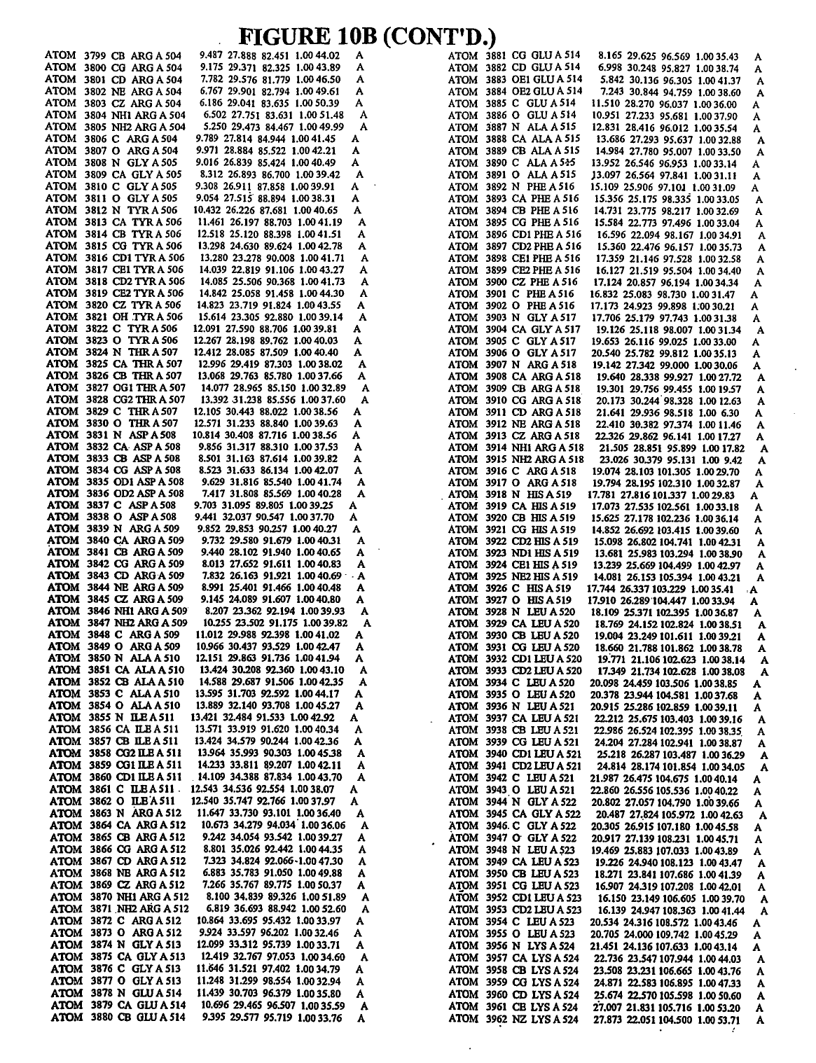

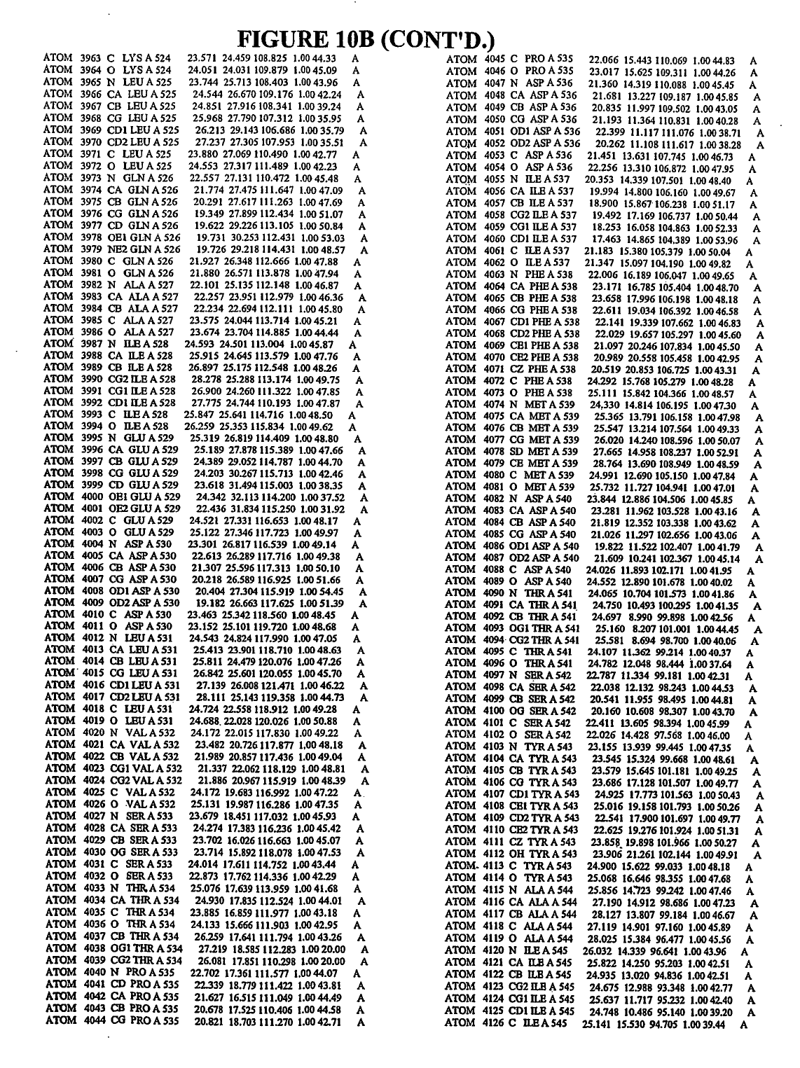

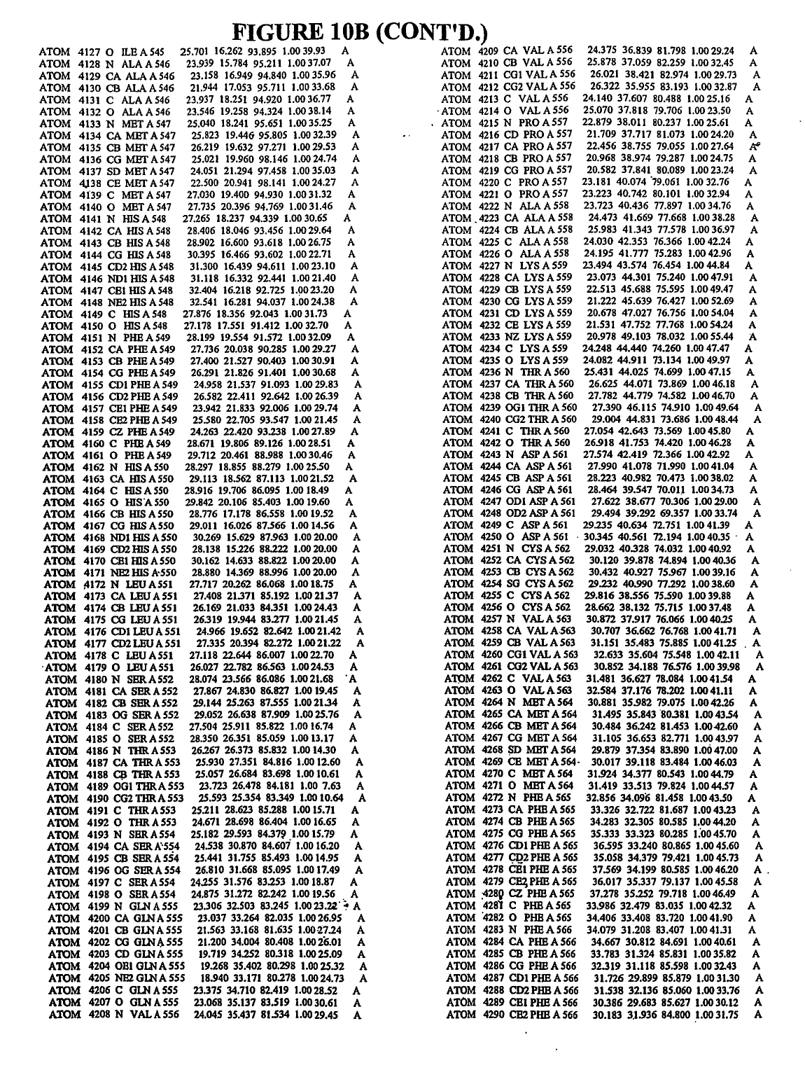

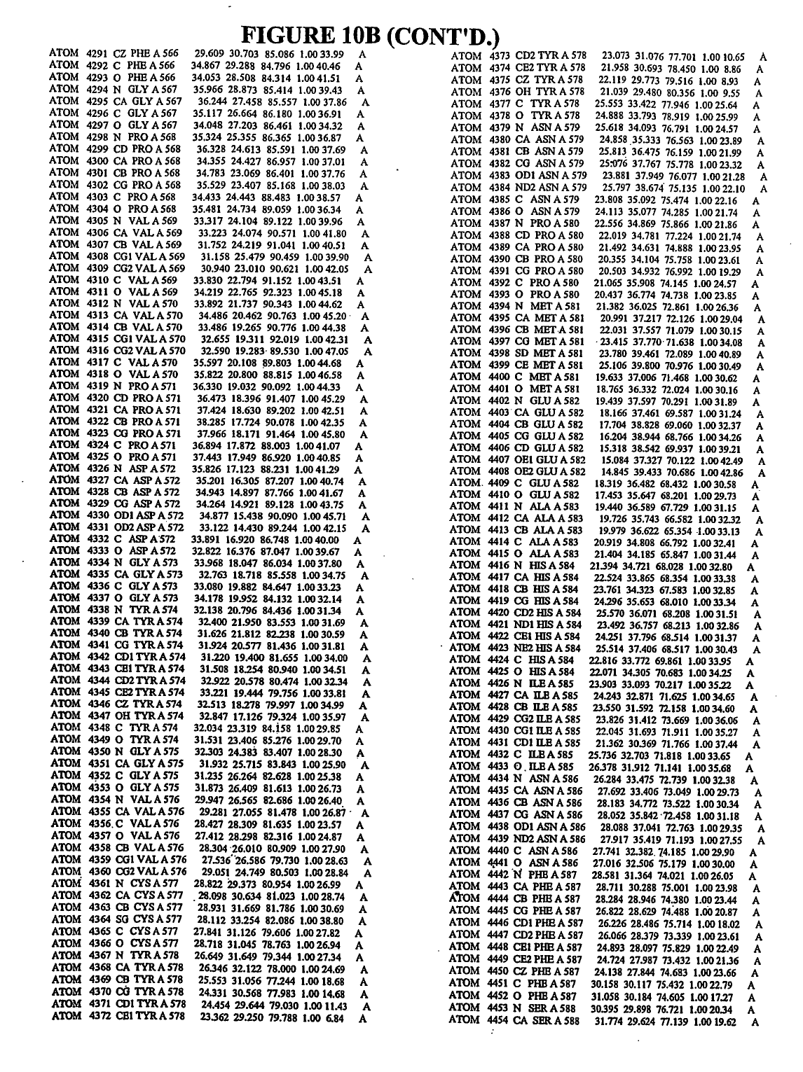

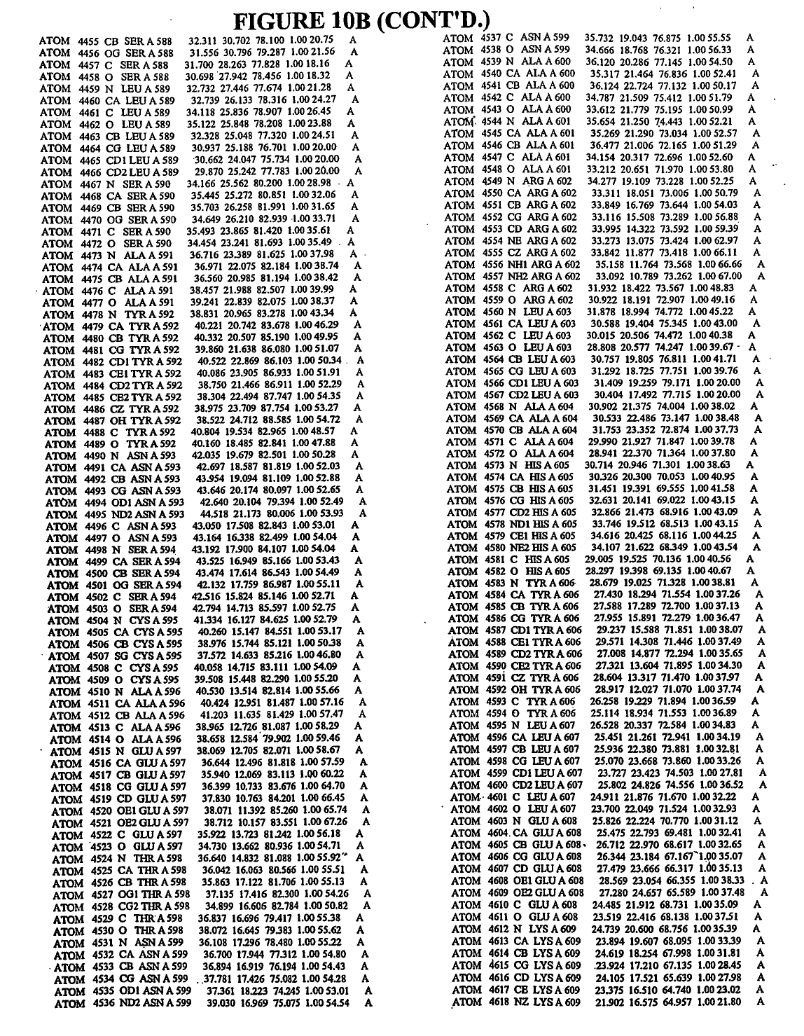

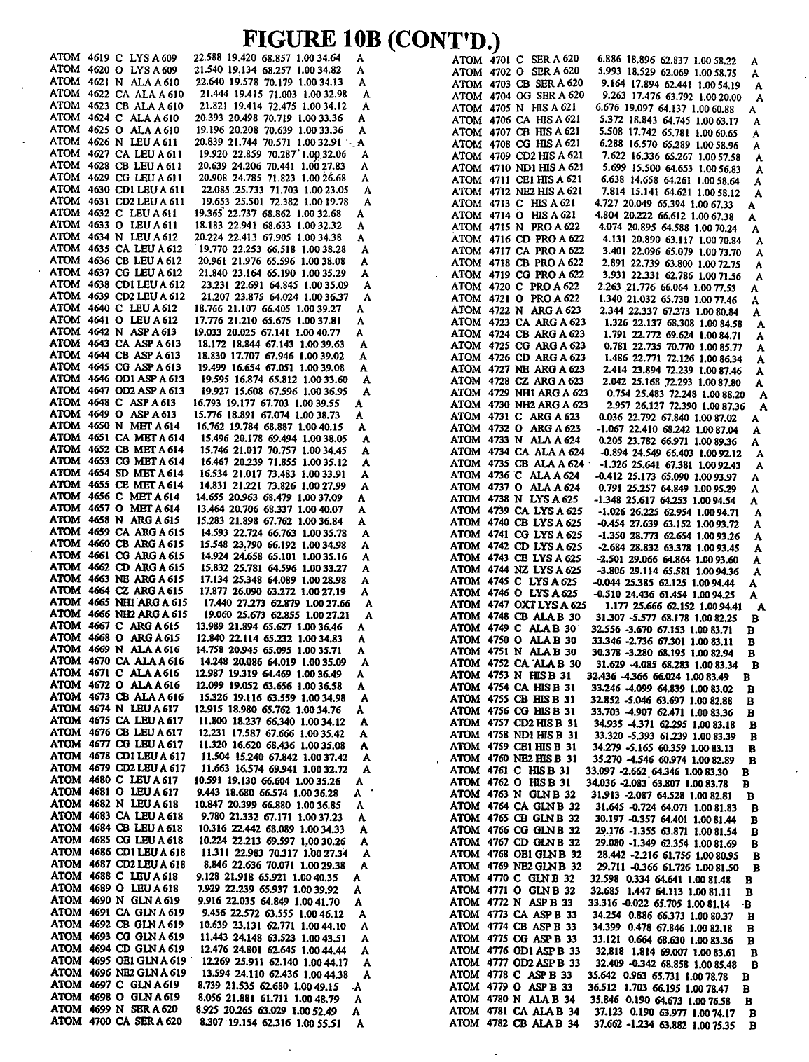

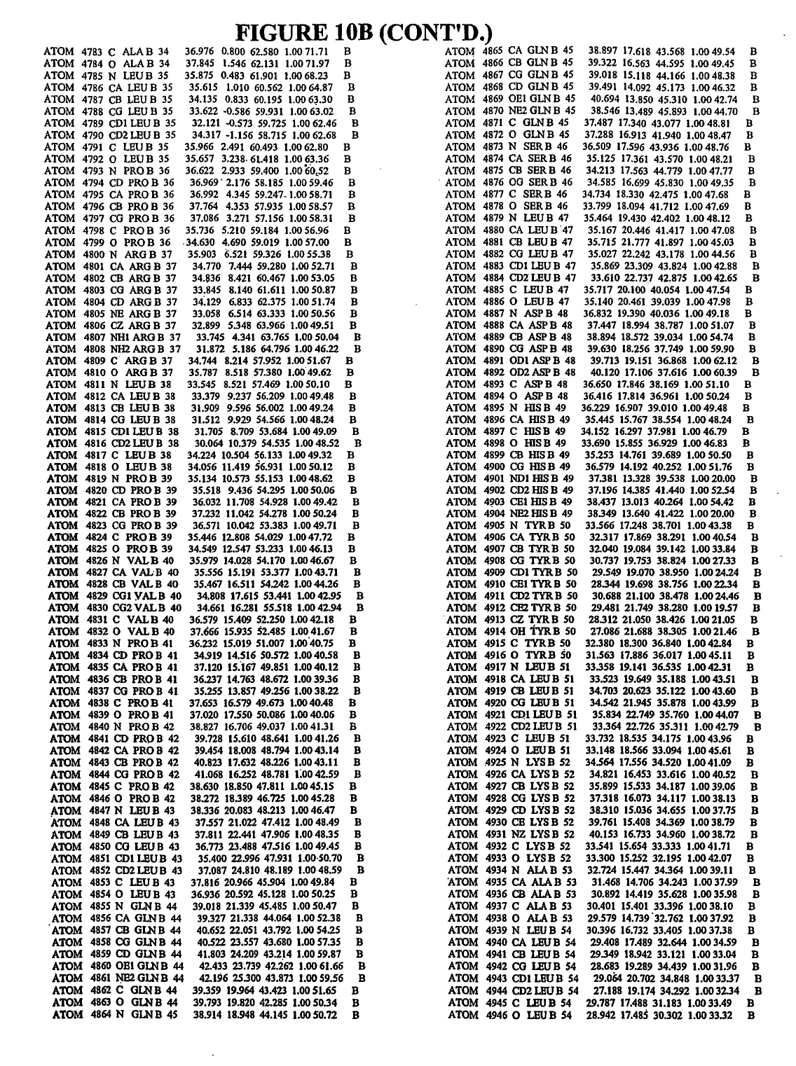

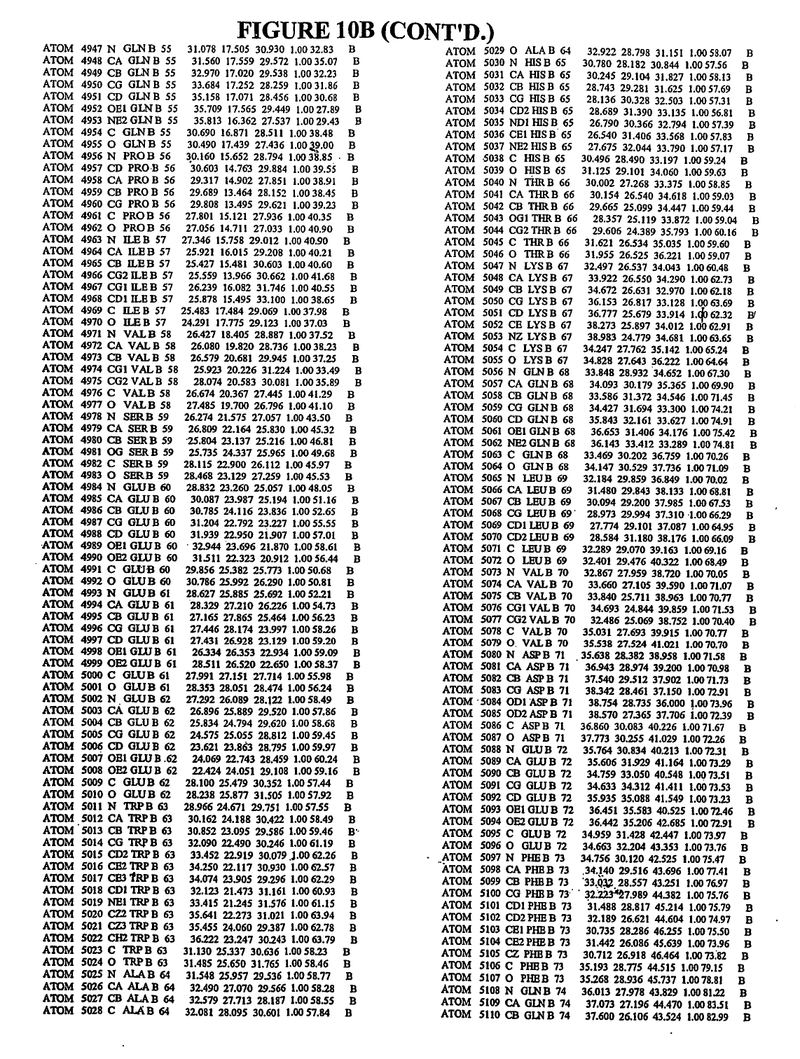

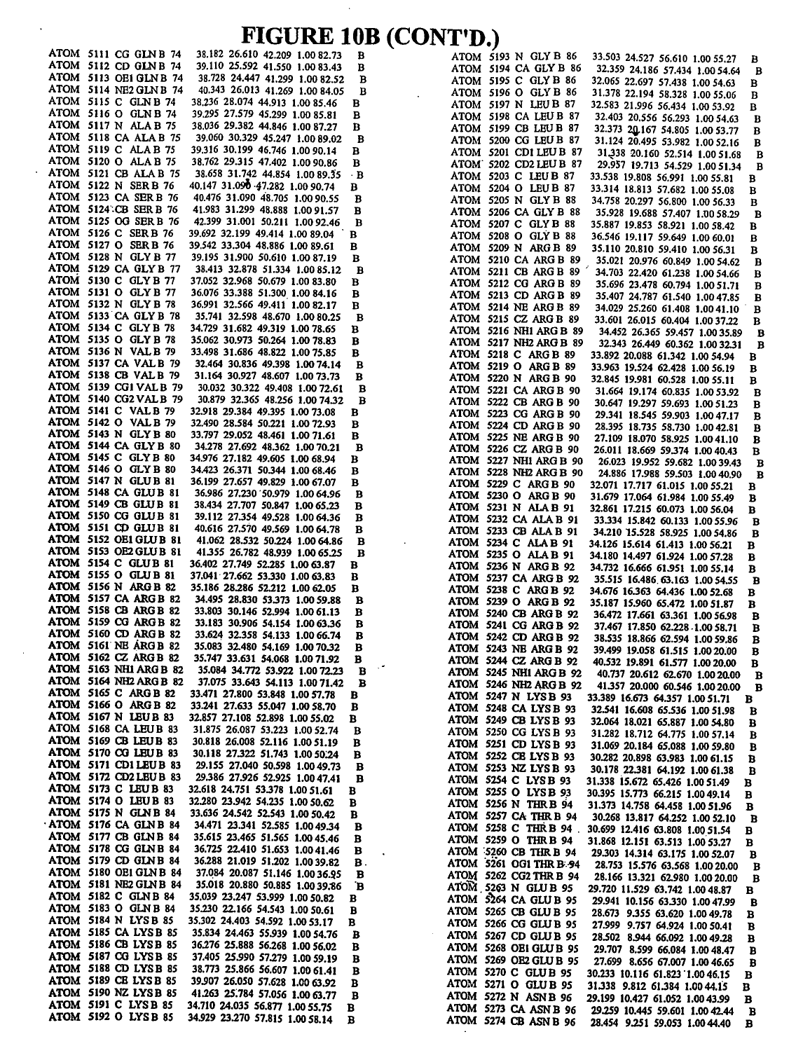

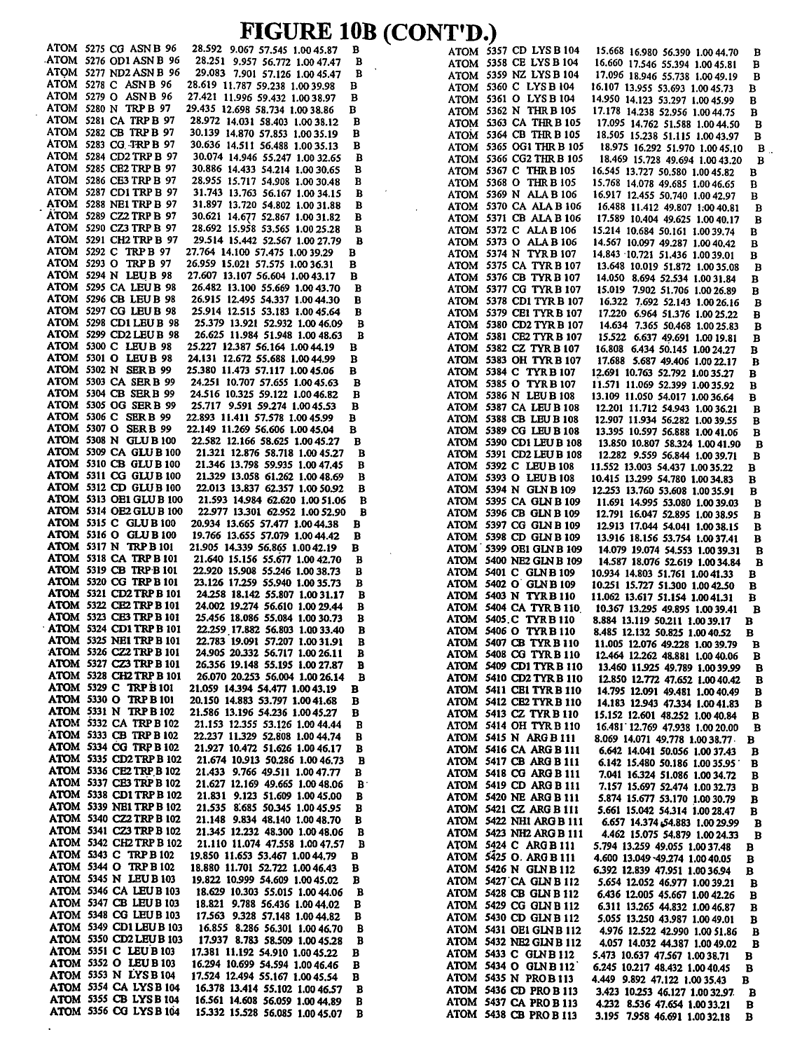

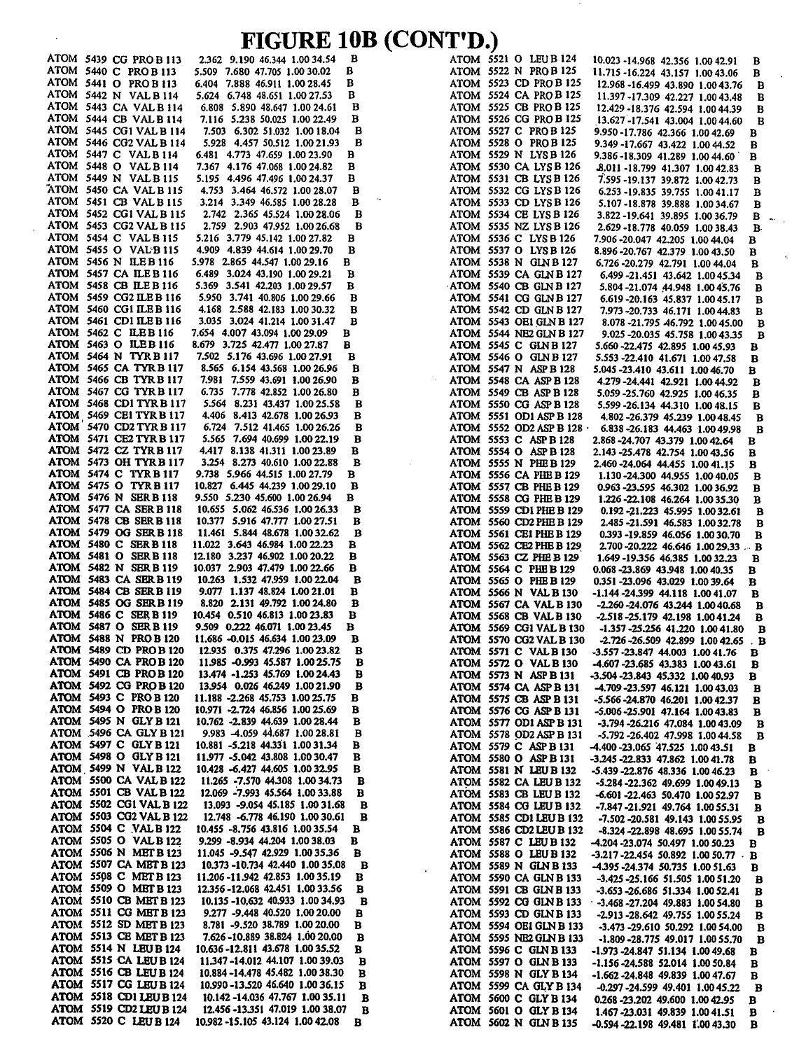

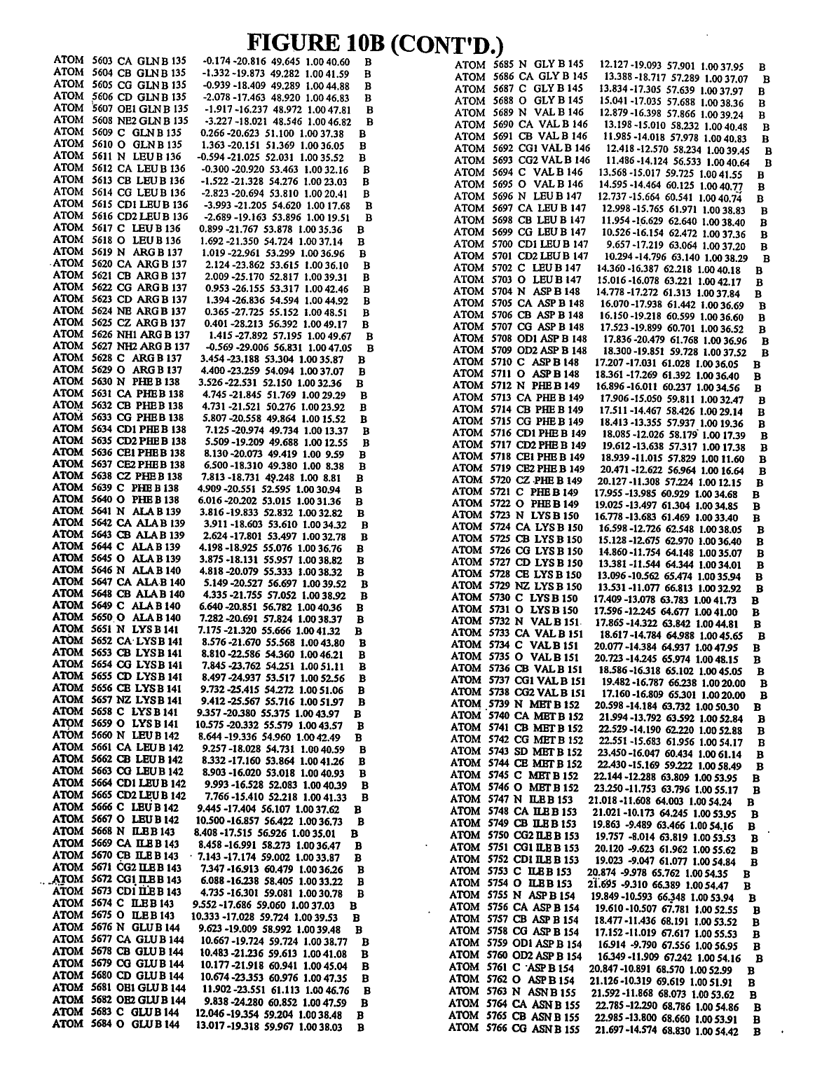

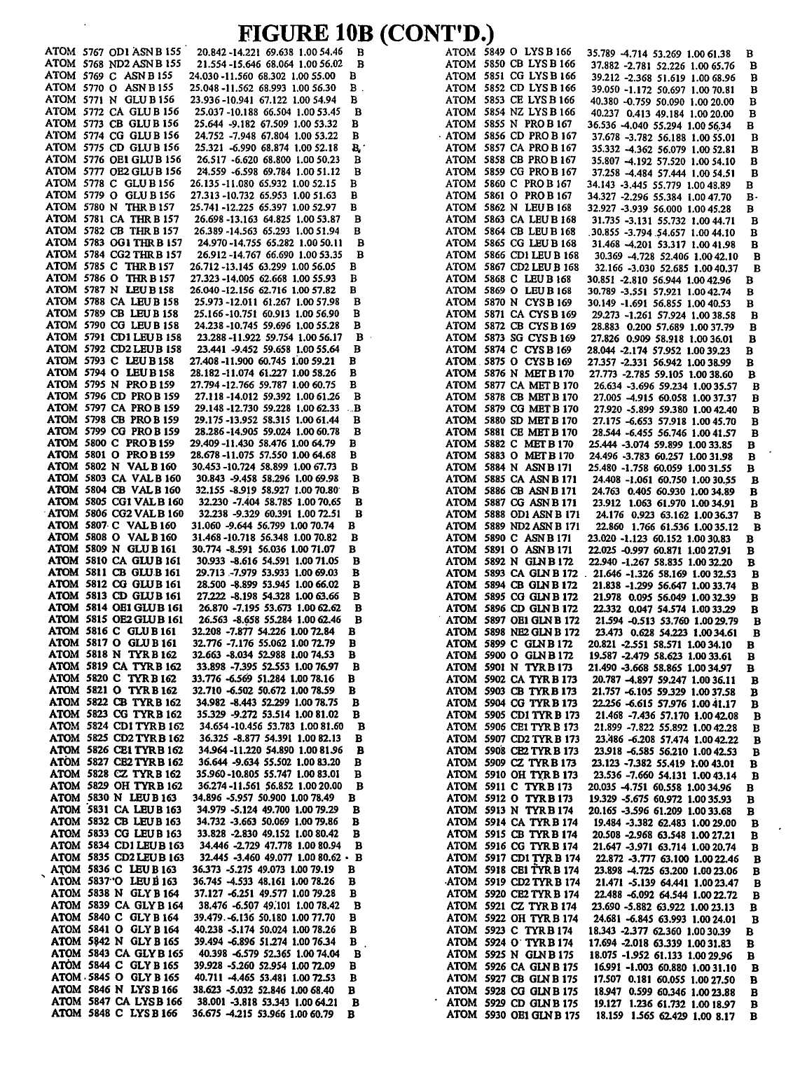

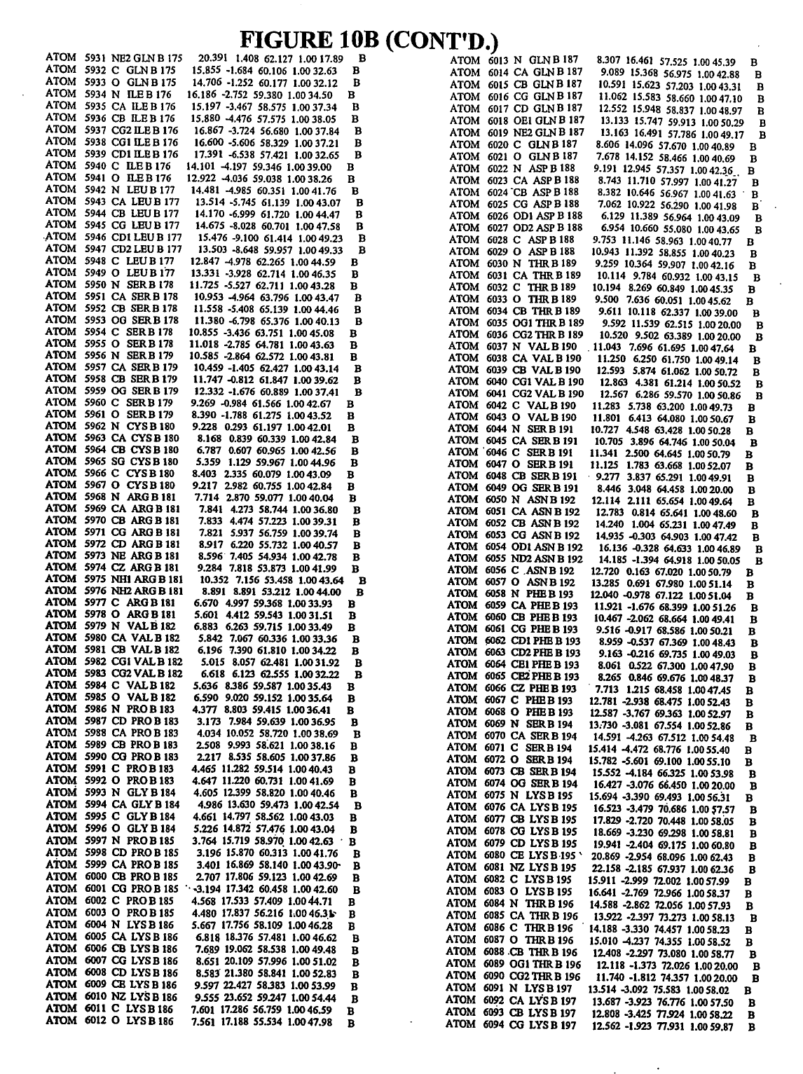

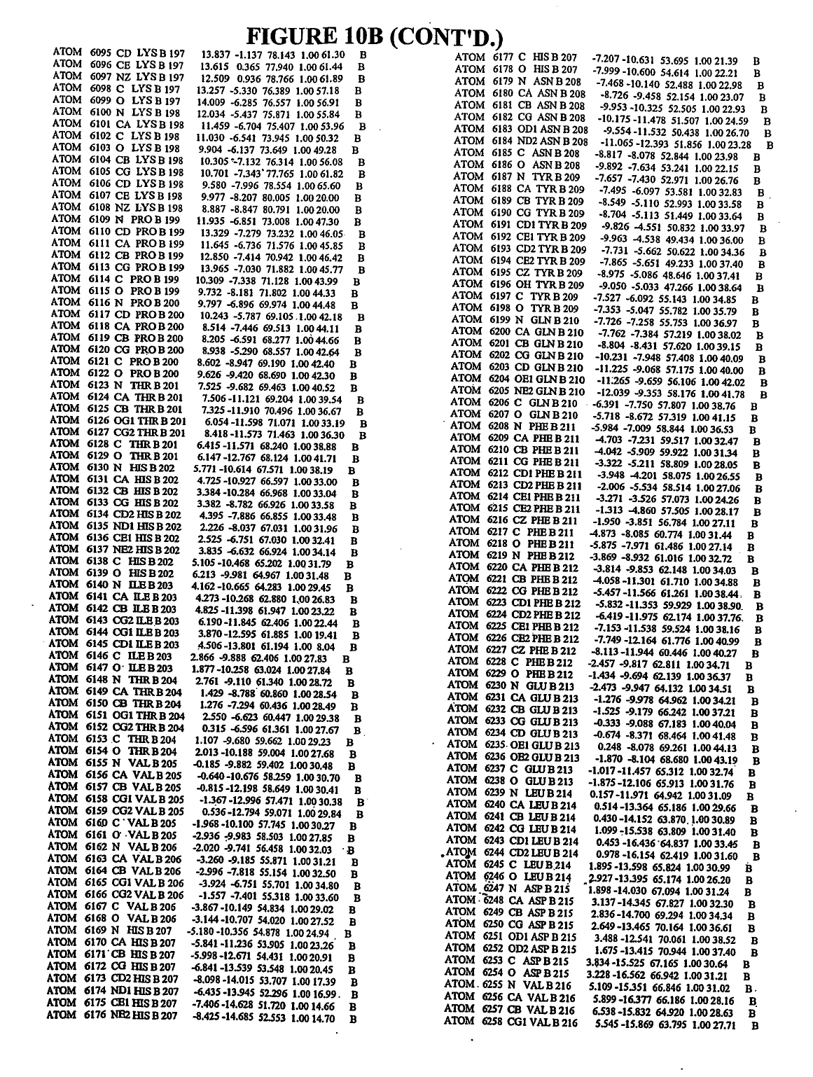

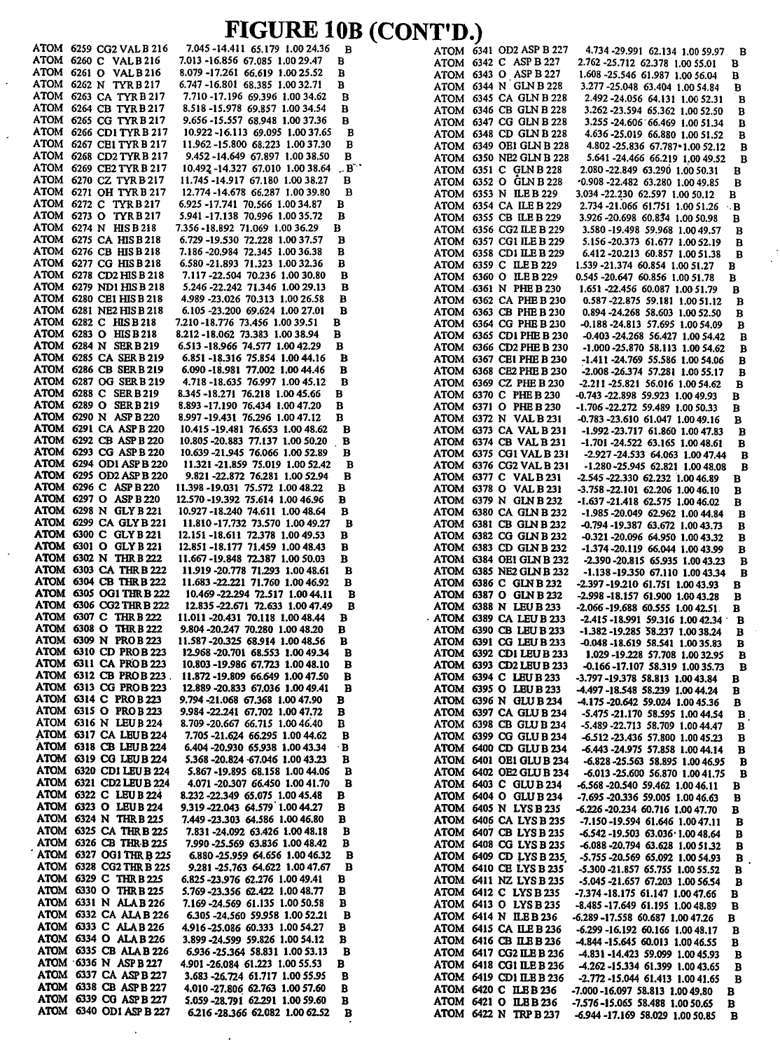

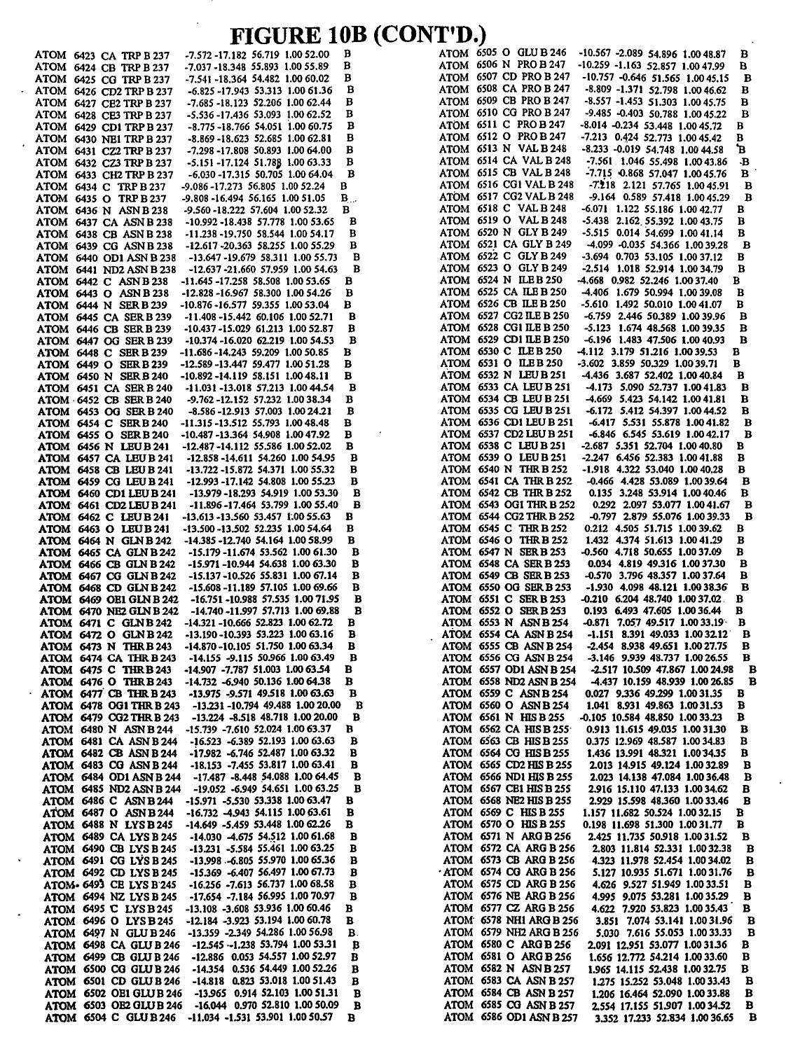

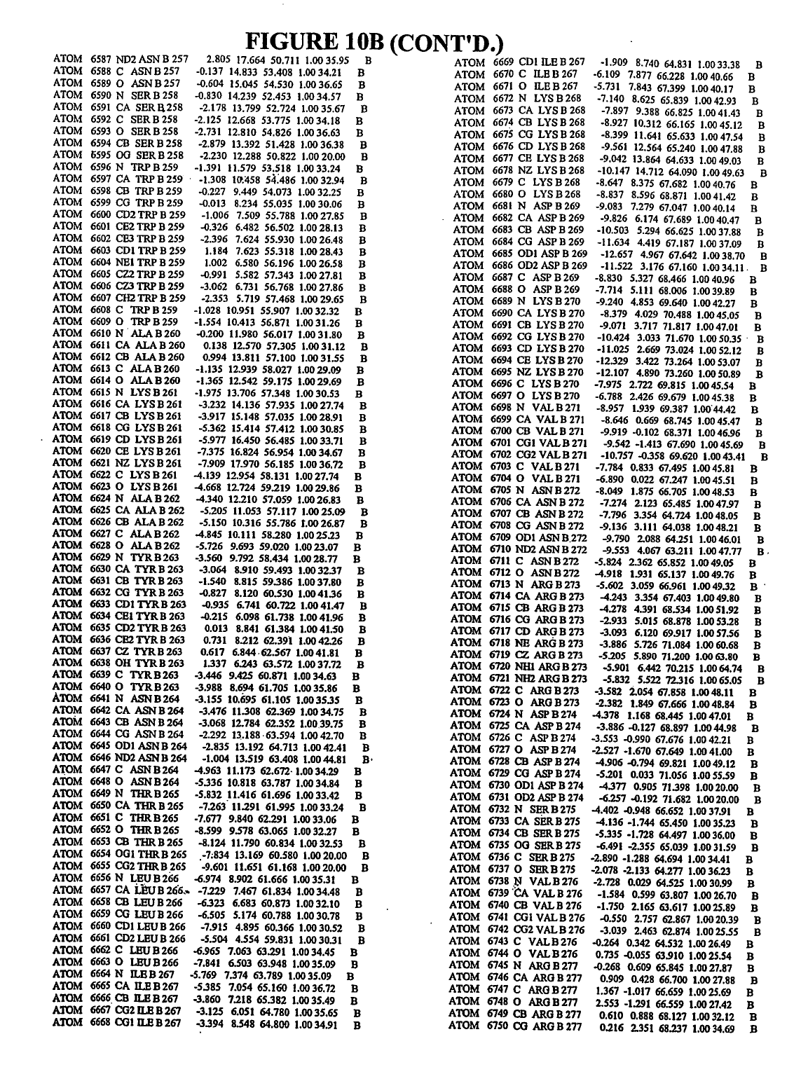

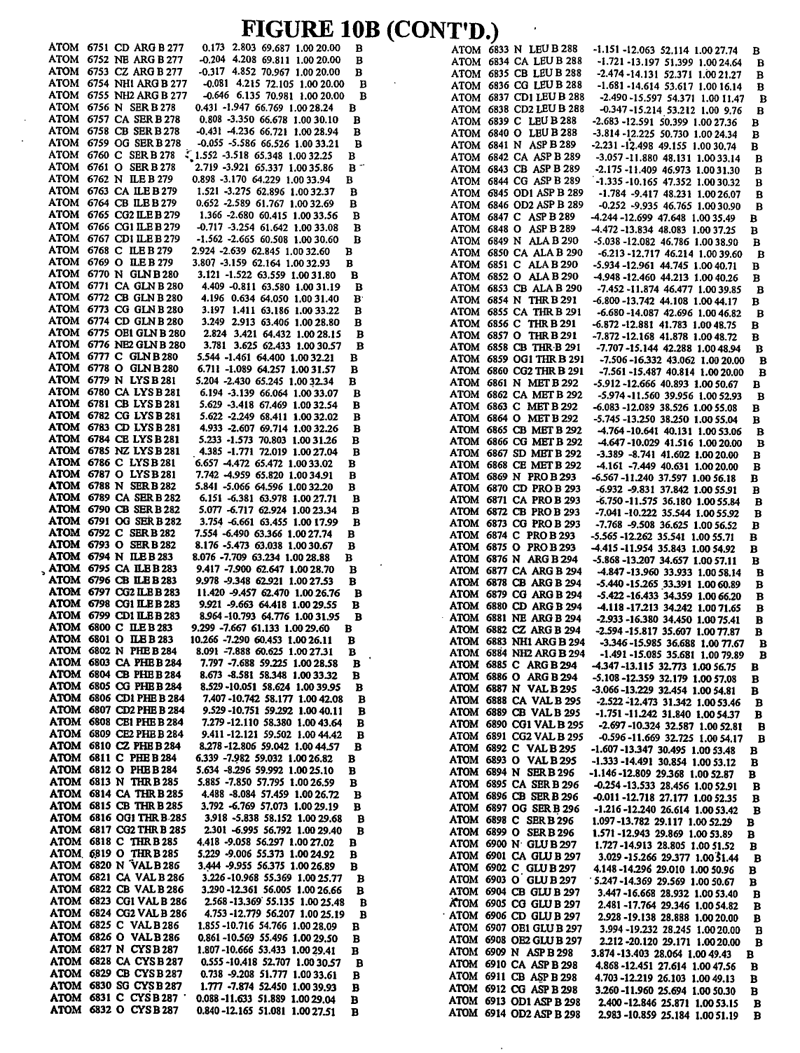

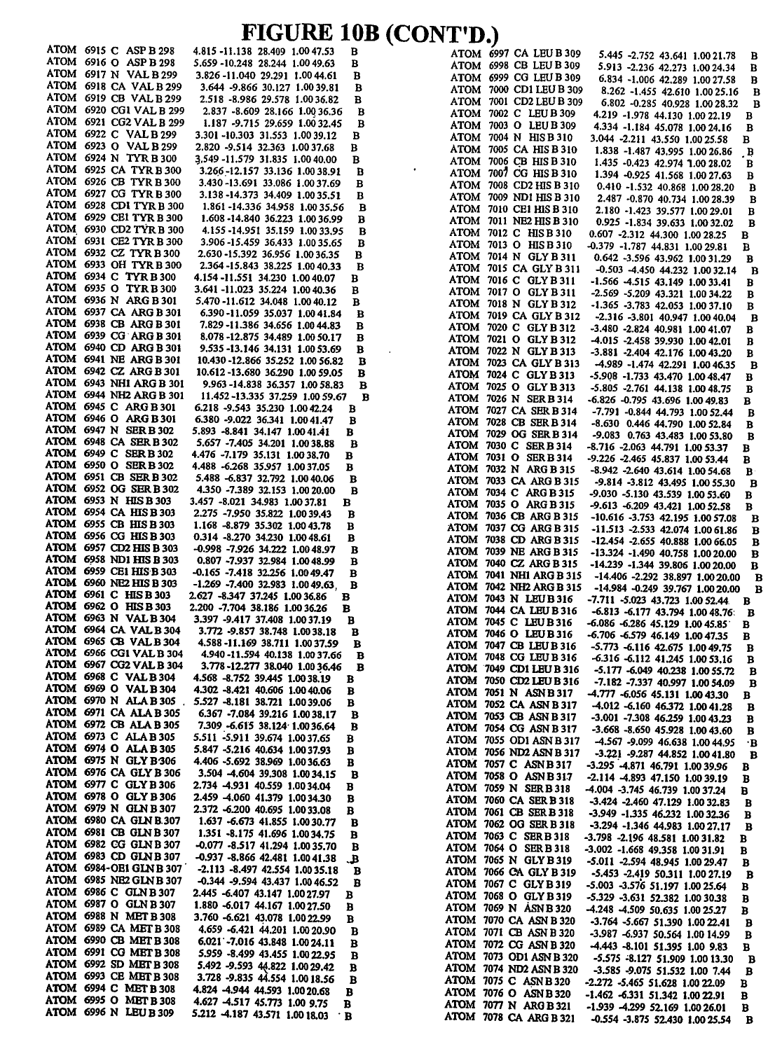

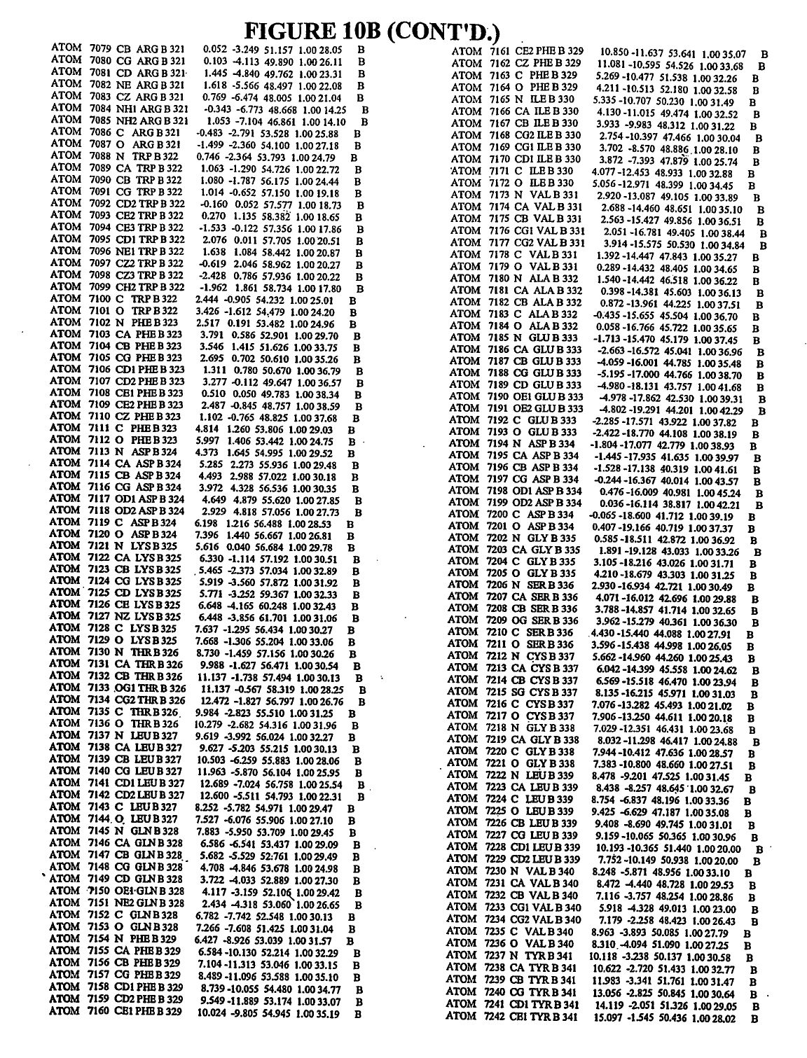

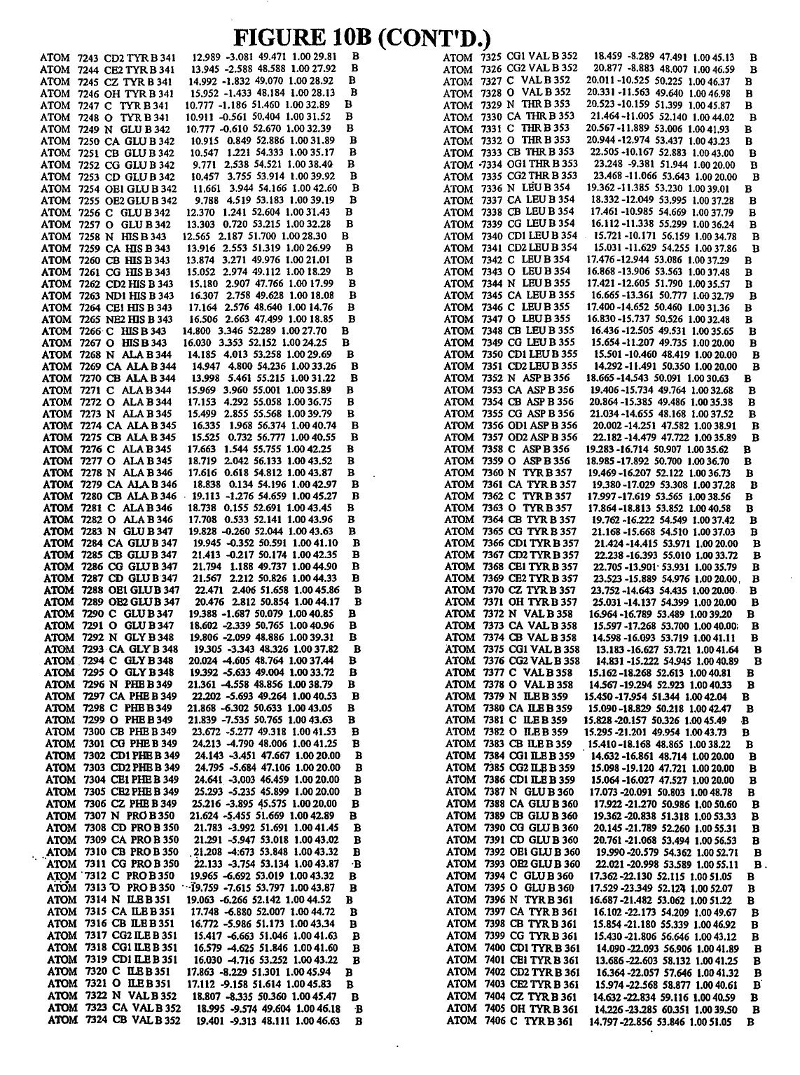

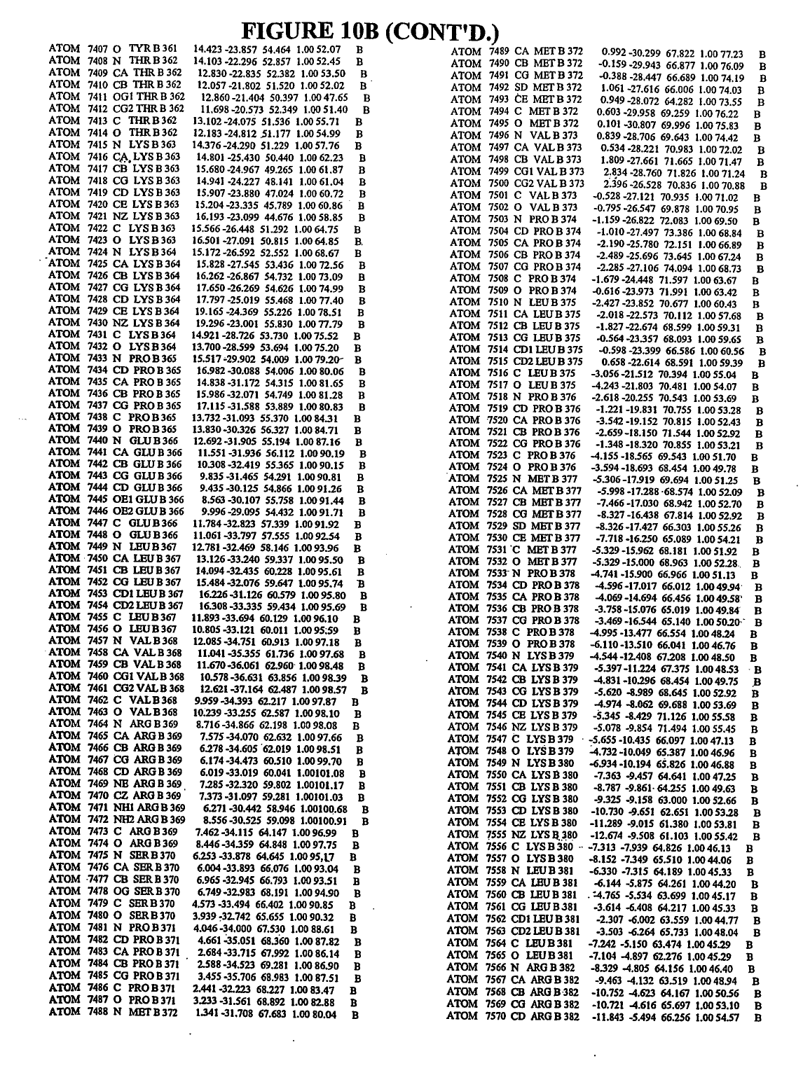

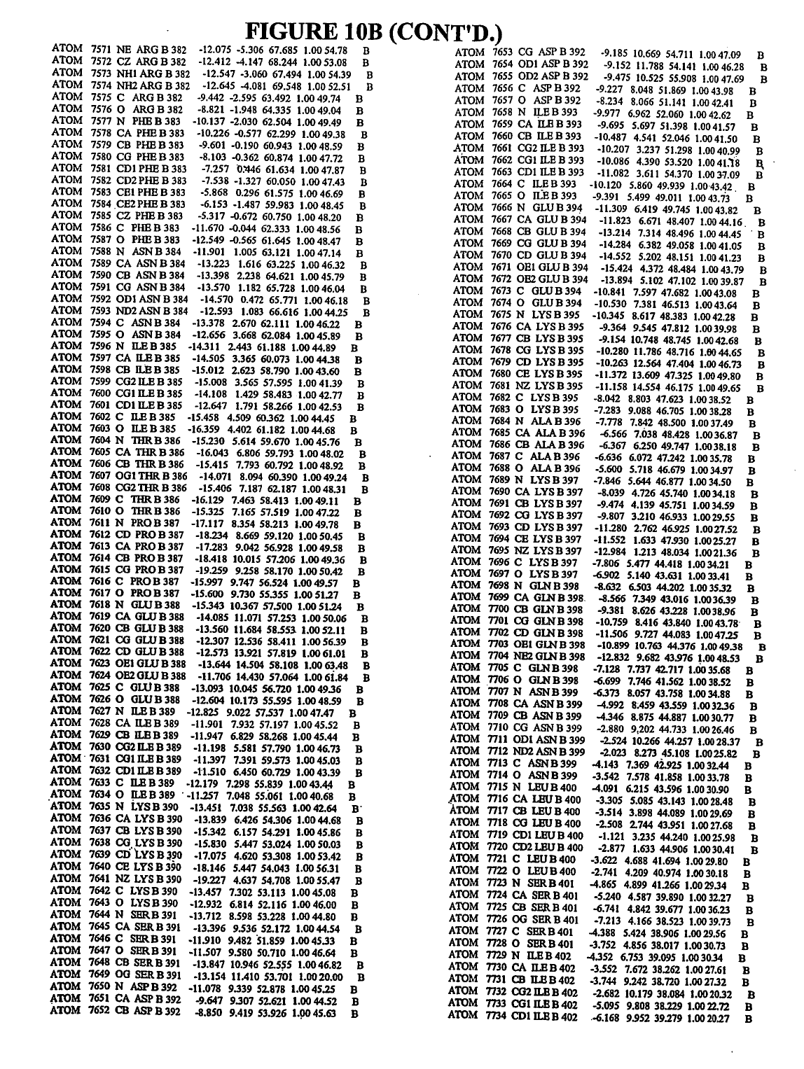

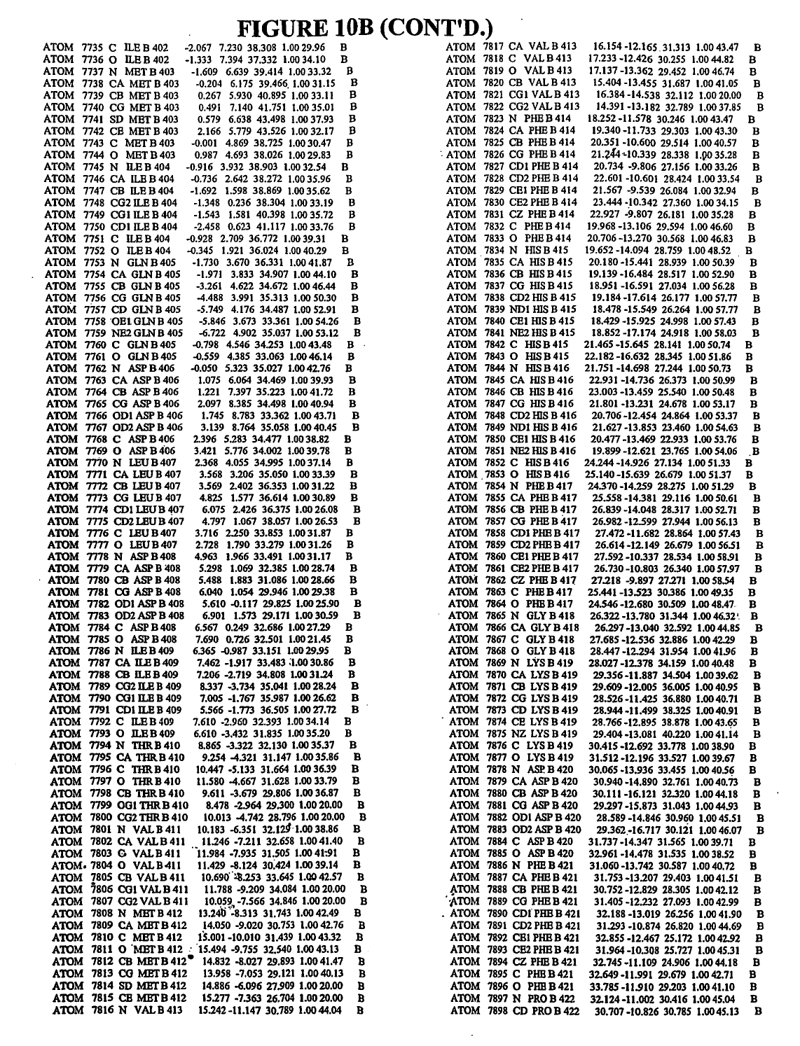

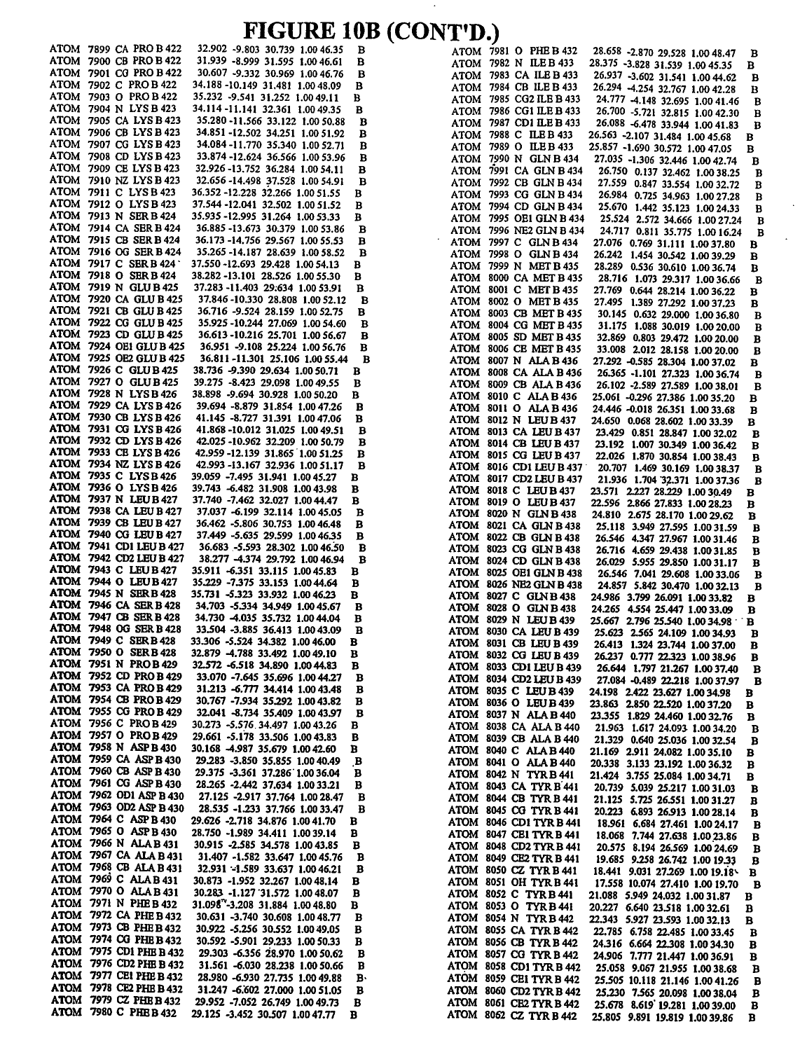

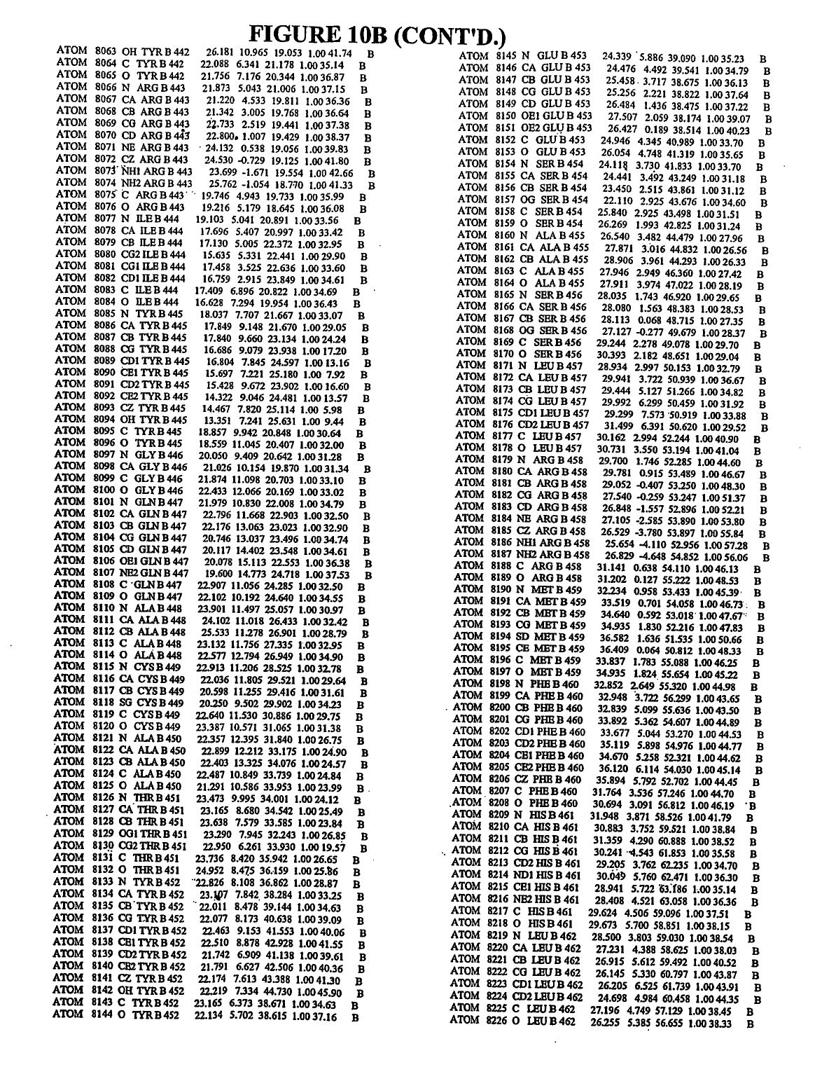

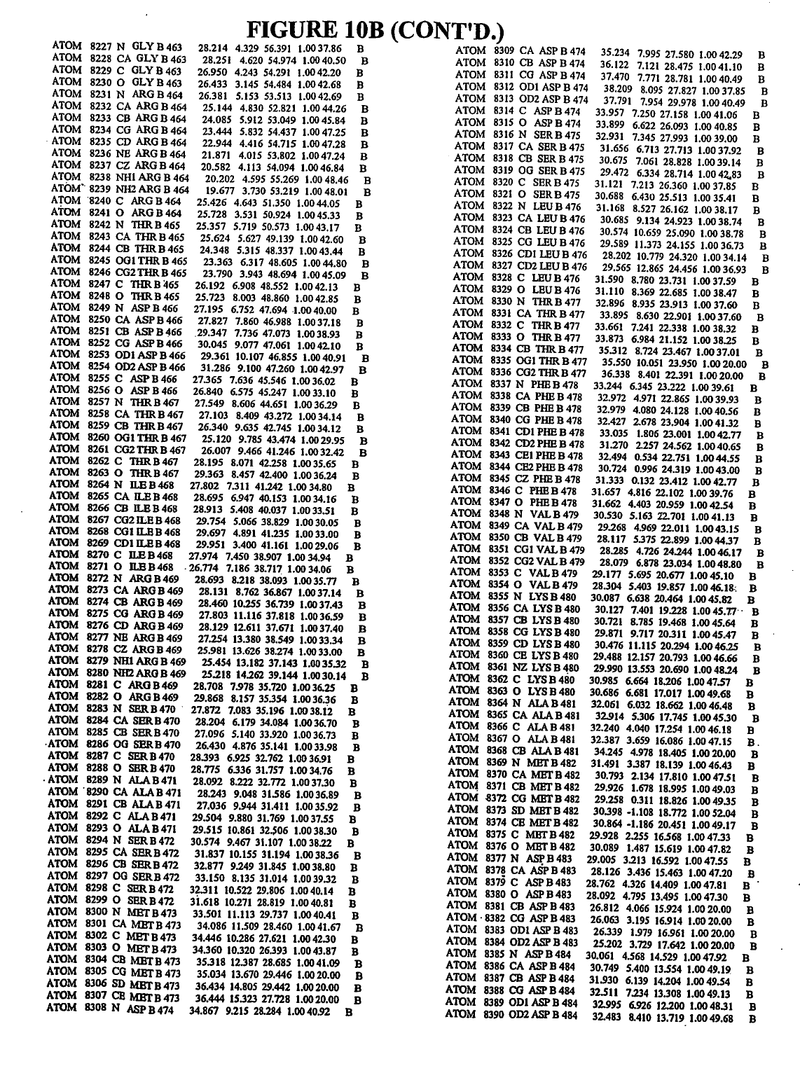

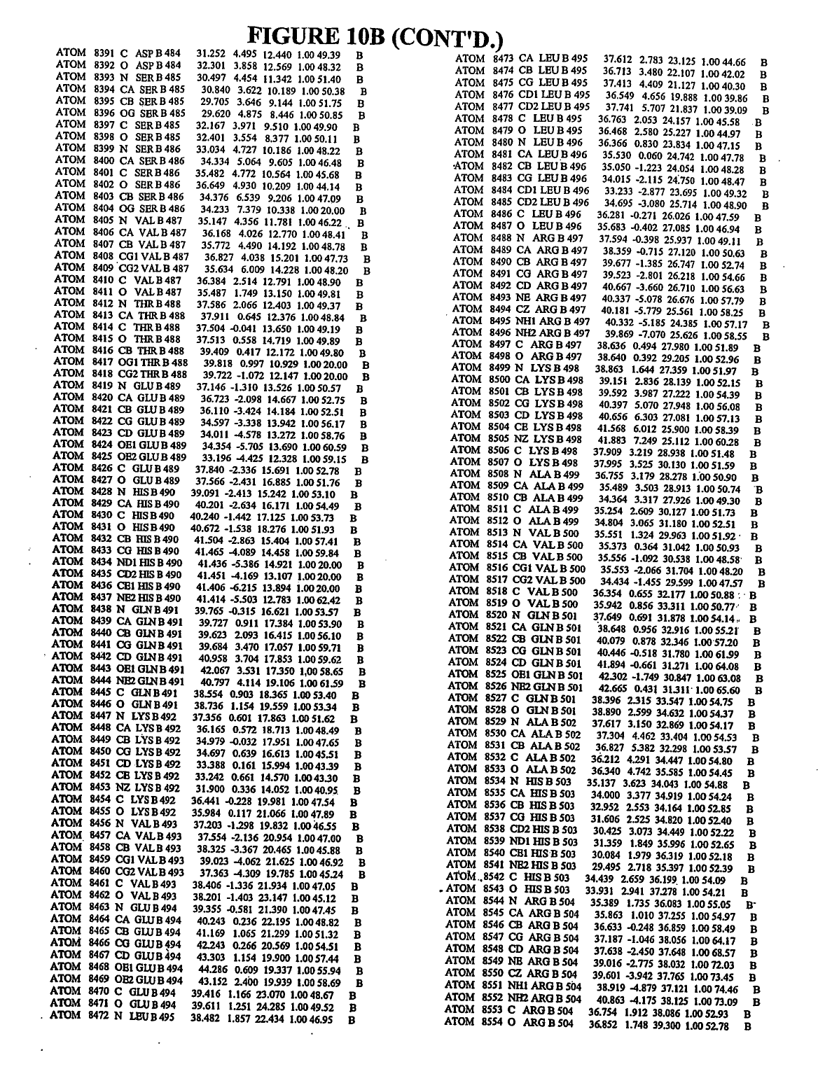

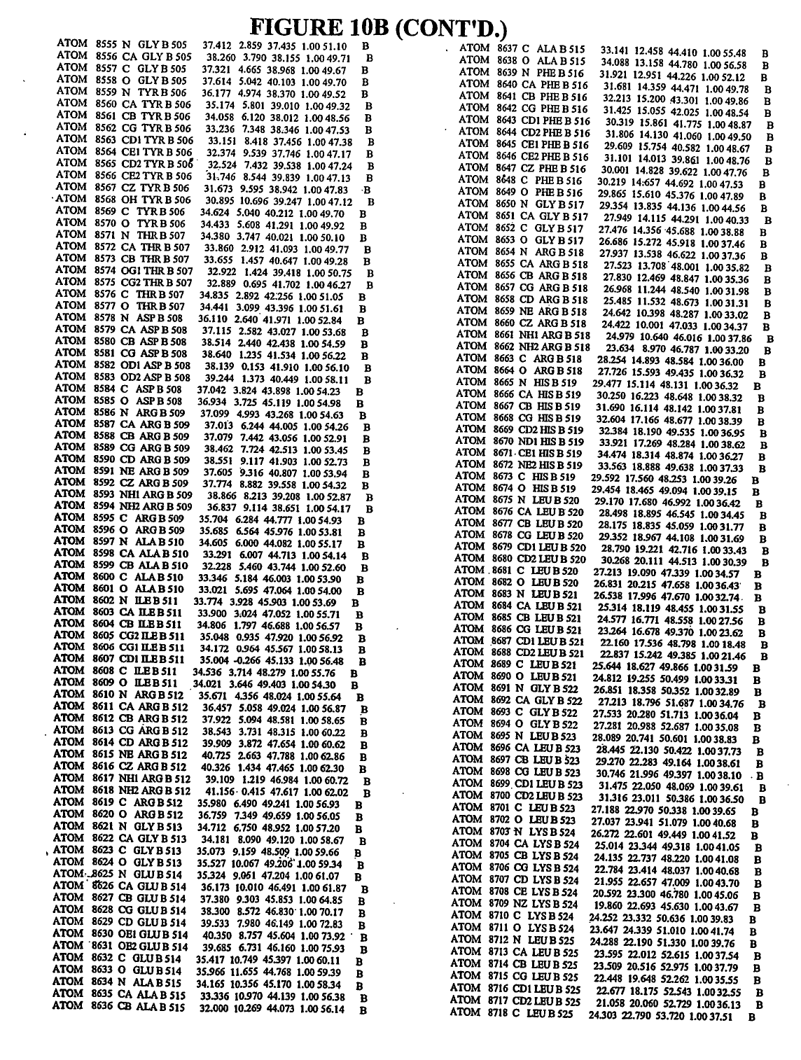

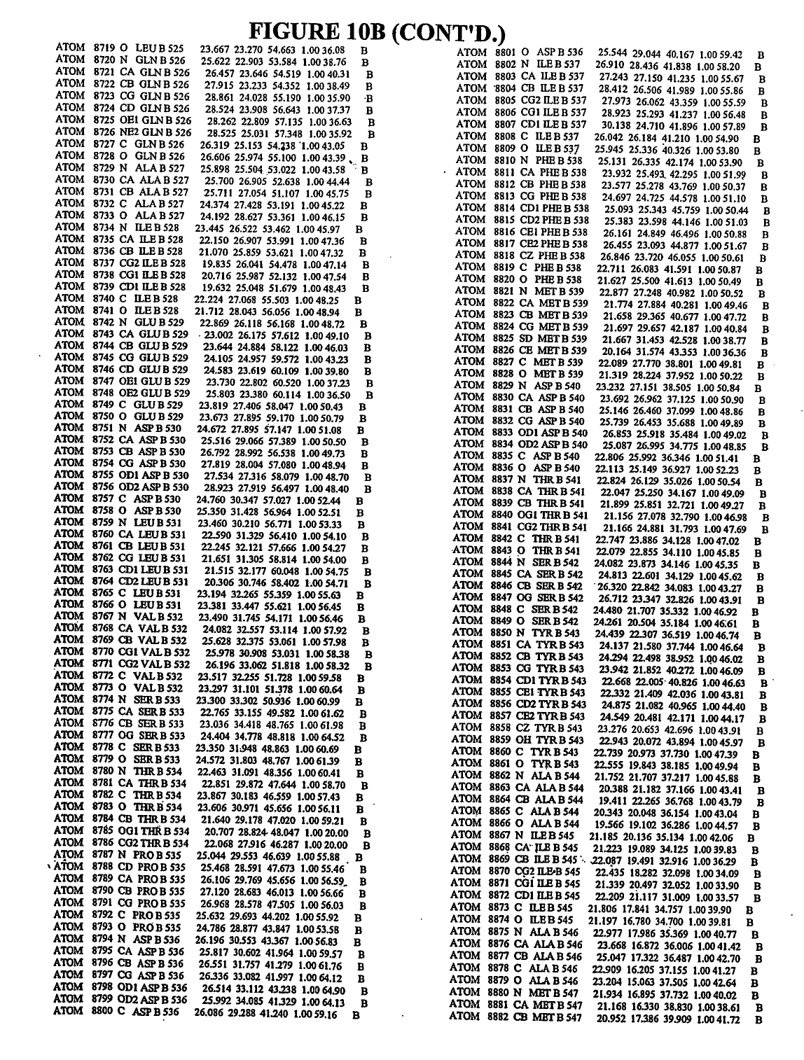

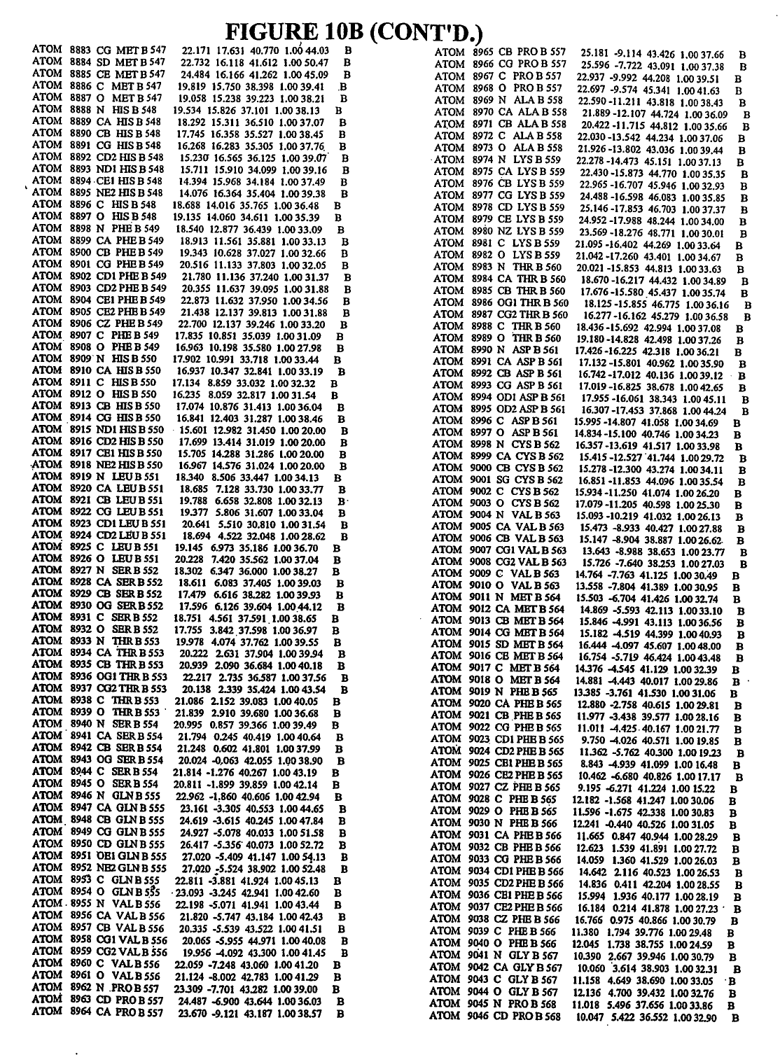

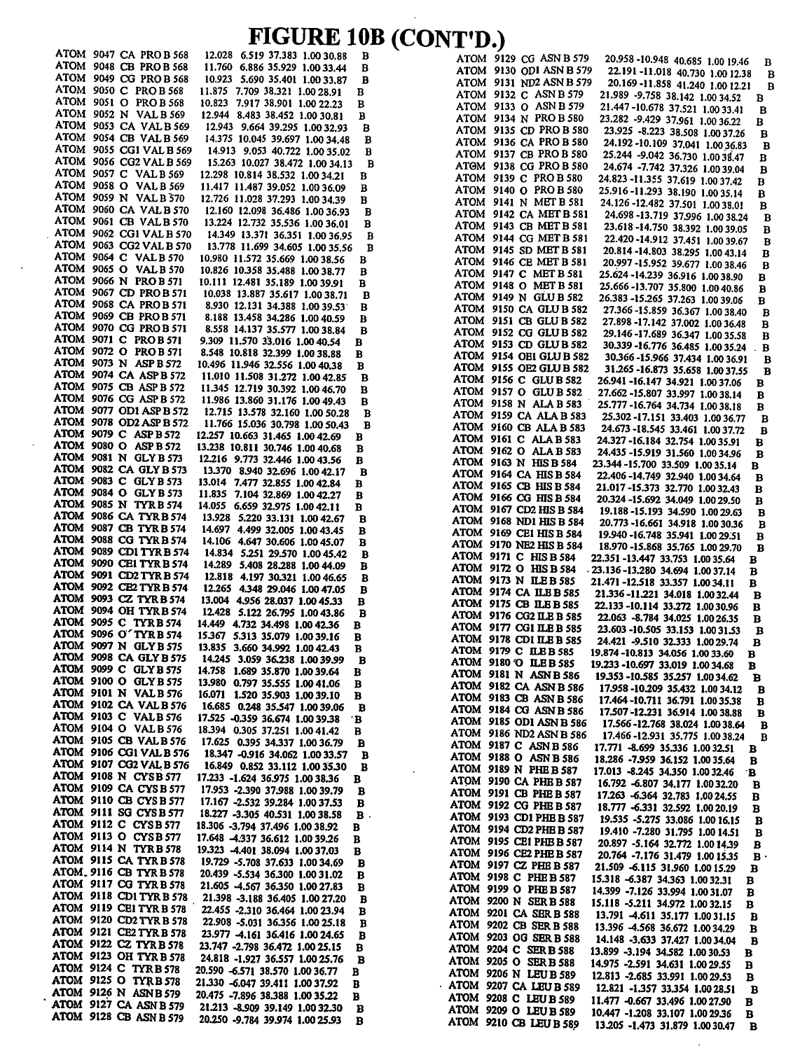

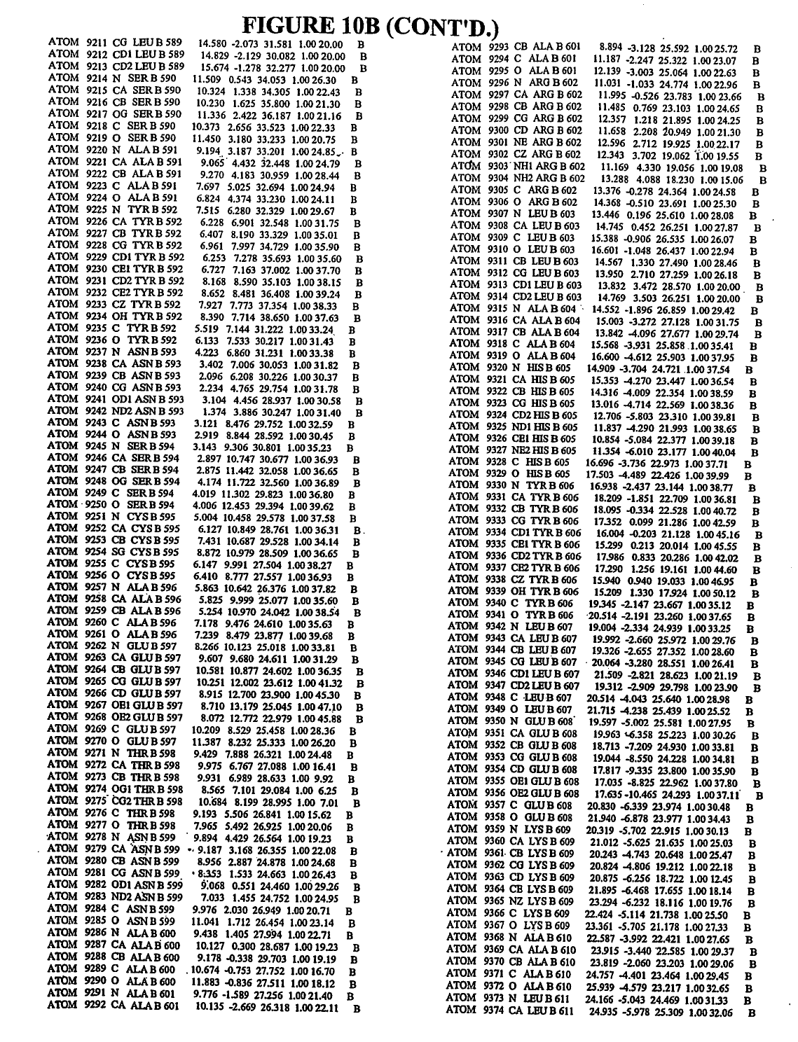

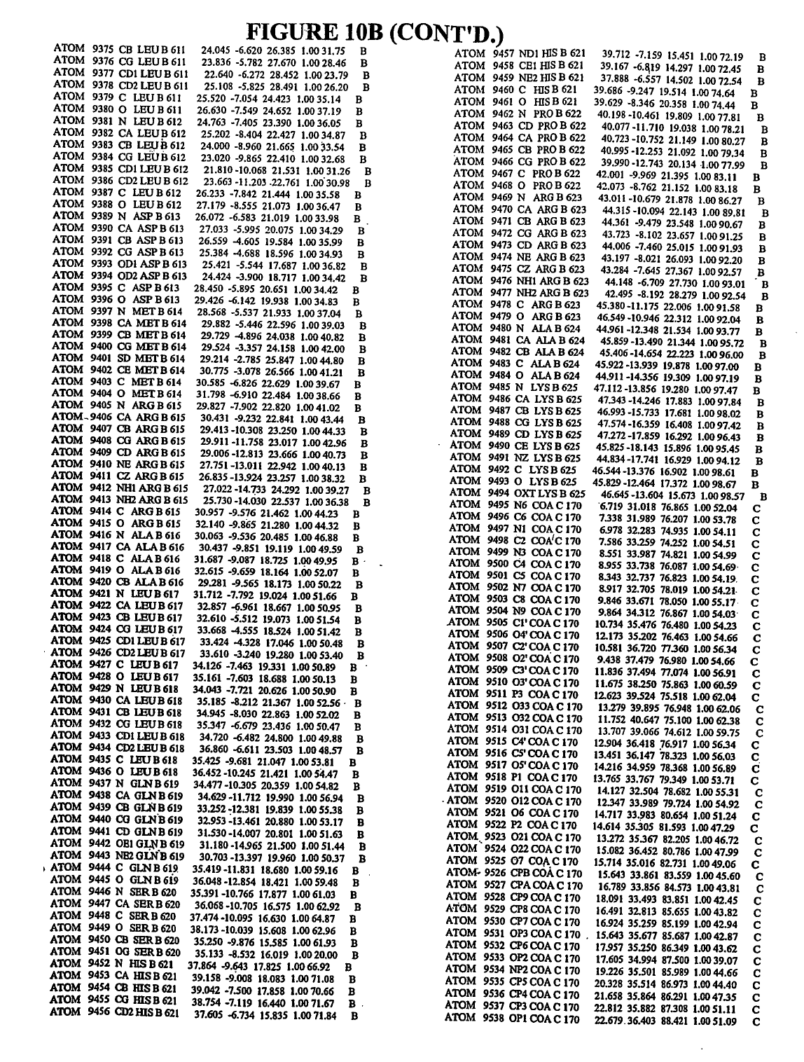

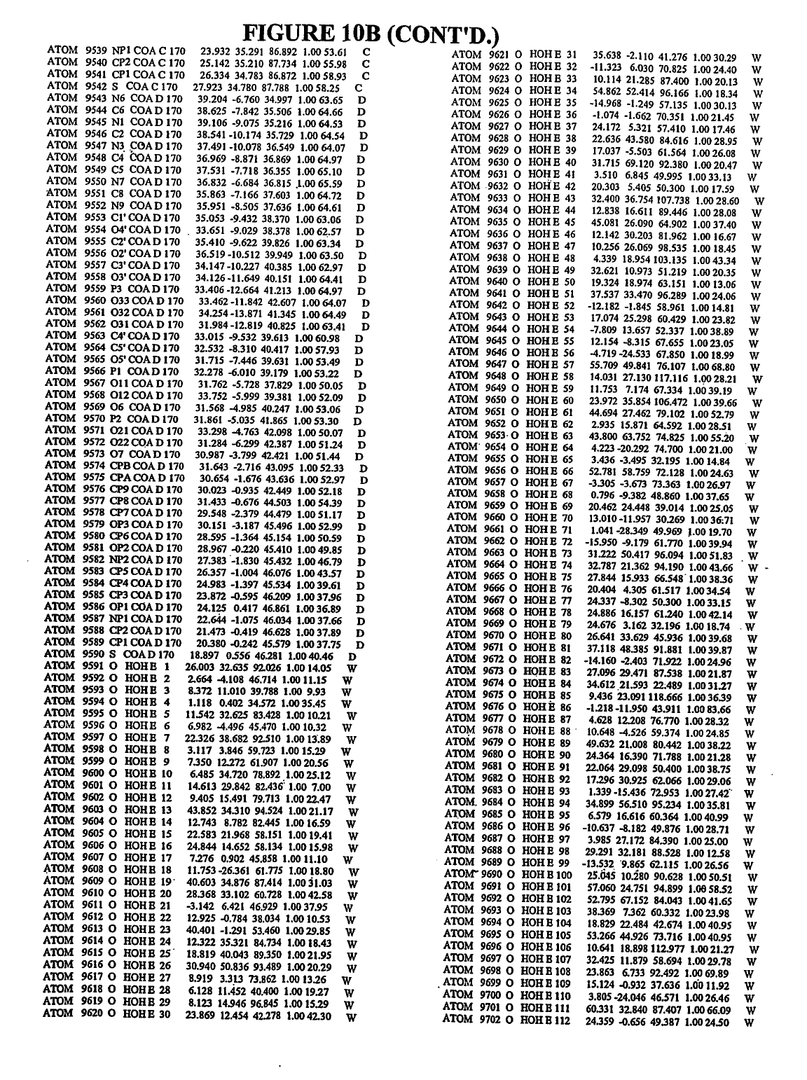

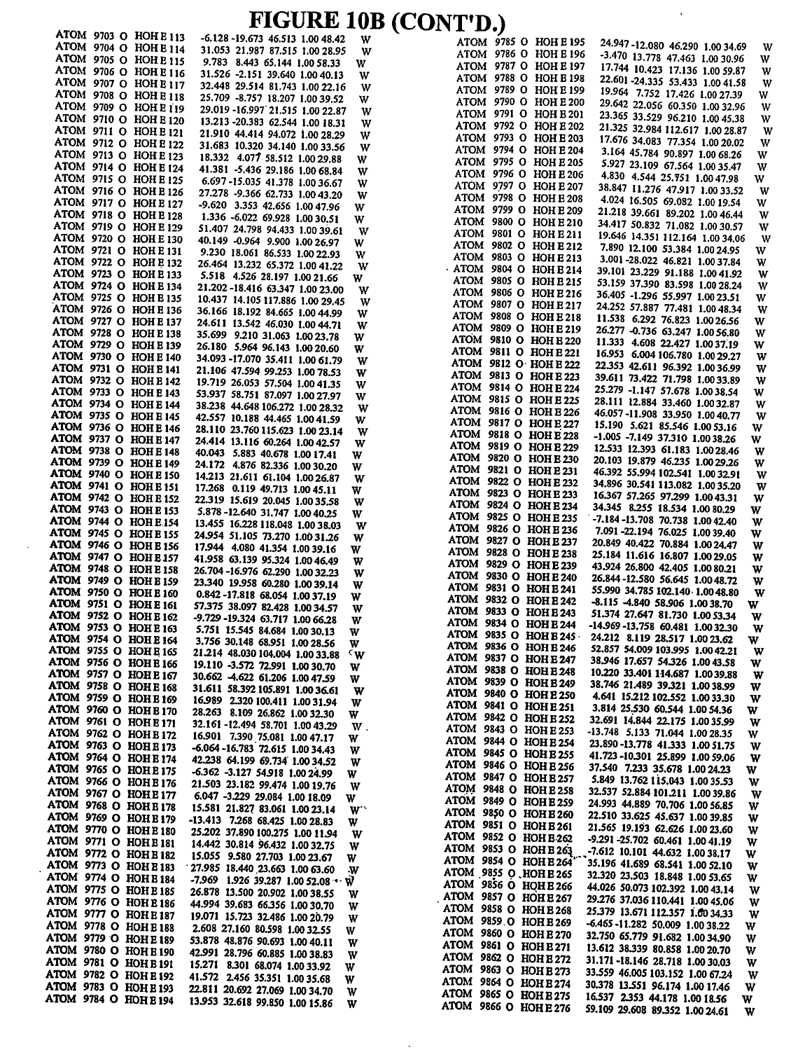

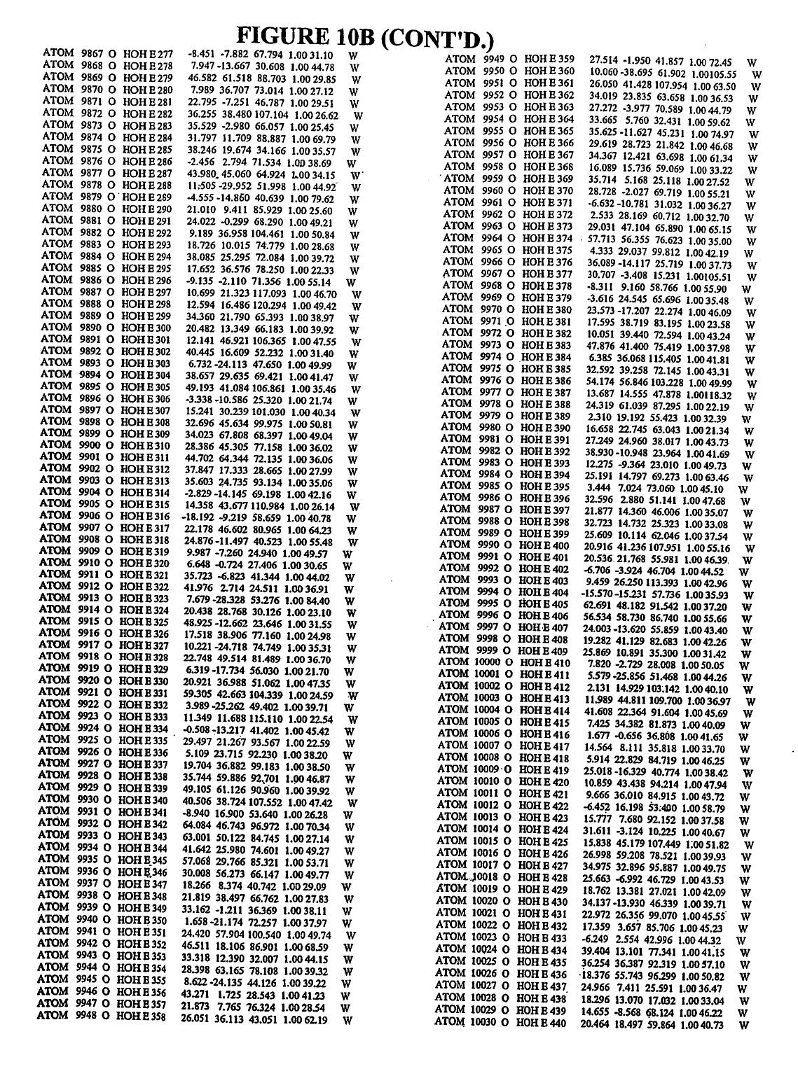

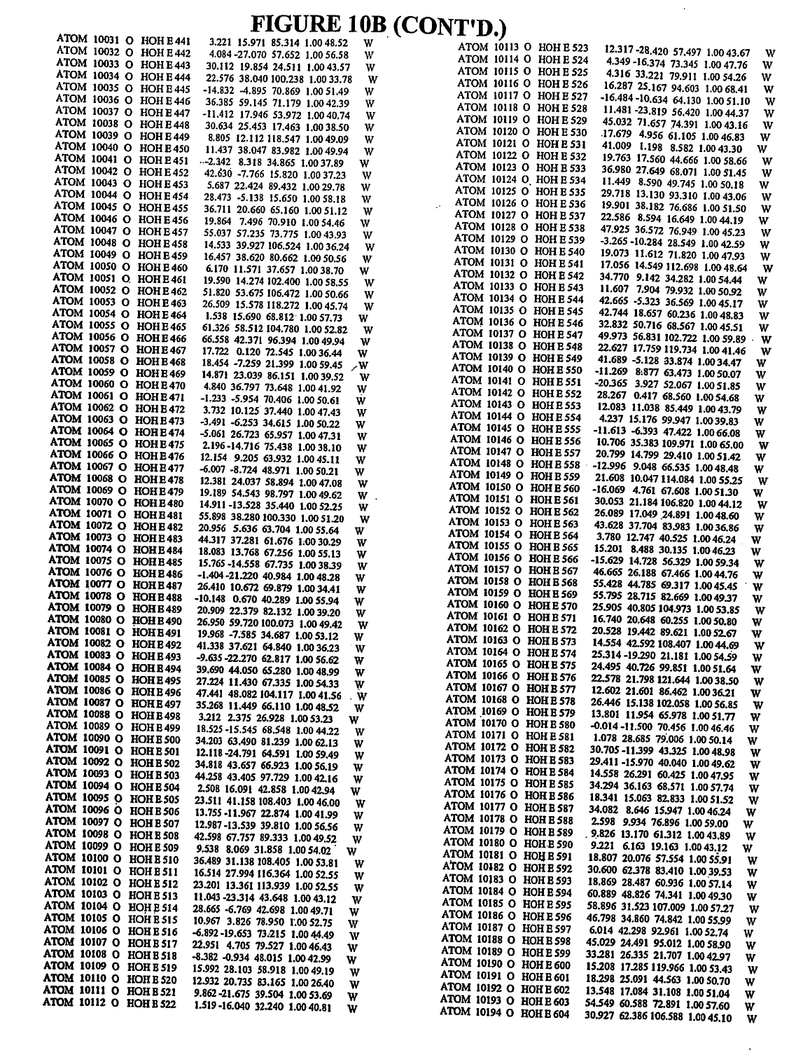

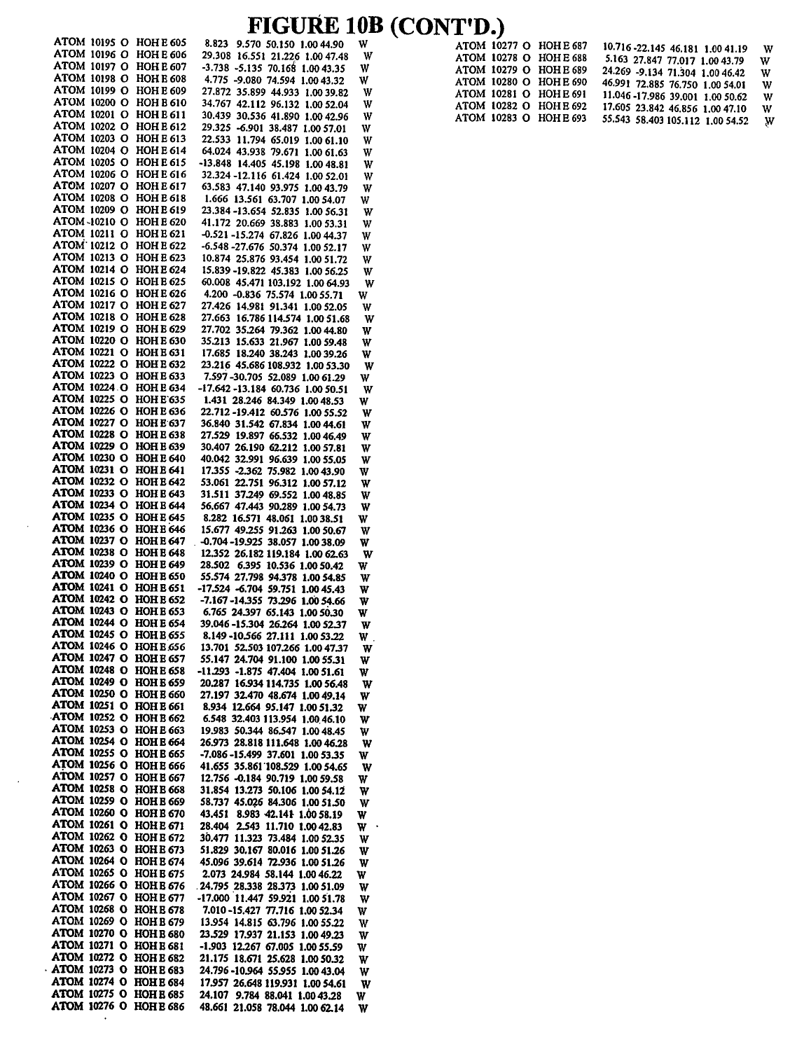

FIG. 10 A lists the atomic coordinates of hCRAT:carnitine, and B lists the atomic coordinates of hCRAT:CoA.

### DETAILED DESCRIPTION OF THE INVENTION

In general, it is the object of the present invention to provide detailed three-dimensional structural information for the family of carnitine acyltransferases, and particularly CRAT. It is also an object of the present invention to provide three-dimensional structural information of carnitine acyltransferases, particularly CRAT, bound to substrates, including, but not limited to, CRAT bound to carnitine and/or CoA.

For purposes of clarity, and not by way of limitation, the detailed description is divided into the following subsections:
a. determination of crystal structure;
b. design of modulators;
c. screening for modulators; and
d. assay systems.

### Determination of Crystal Structure

The three dimensional structure of a carnitine acyltransferase may be determined by obtaining its crystal structure directly and/or by comparing the primary and/or secondary structure of the carnitine acyltransferase, and/or an incomplete set of components of its three dimensional structure, with a crystal structure which has already been solved.

The three-dimensional structural information obtained from crystals of mCRAT, mCRAT:carnitine and mCRAT:CoA may be employed to solve the structures of other CRAT species, including but not limited to hCRAT, as well as the structures of other carnitine acyltransferases. The atomic coordinates calculated for hCRAT are set forth in FIG. 10, and a comparison of the structures of mCRAT and hCRAT shows that the structures are very similar.

Structural information is desirably obtained from a pure preparation of a carnitine acyltransferase or from the purified enzyme combined with a substrate thereof. Preferably, the carnitine acyltransferase is comprised in a protein composition where it constitutes greater than 70 percent, and more preferably greater than 90 percent, of the total protein.

The carnitine acyltransferase may be prepared from a natural source, may be produced by recombinant DNA technology, or may be chemically synthesized (although this last possibility would be extremely cumbersome). For example, a full-length cDNA encoding a carnitine acyltransferase such as CRAT may be subcloned from a cDNA preparation by the polymerase chain reaction (PCR), using at least one primer designed based on know, homologous, or obtained protein sequence, and inserted into an expression vector. A large number of suitable vector-host systems are known in the art. Possible vectors include, but are not limited to, plasmids or modified viruses, but the vector system must be compatible with the host cell used. Examples of vectors include E. coli bacteriophages such as lambda derivatives, or plasmids such as pBR322 derivatives or pUC plasmid derivatives, e.g., pGEX vectors (Amersham-Pharmacia, Piscataway, N.J.), pET vectors (Novagen, Madison, Wis.), pmal-c vectors (Amersham-Pharmacia, Piscataway, N.J.), pFLAG vectors (Chiang and Roeder, 1993, Pept. Res. 6:62-64), baculovirus vectors (Invitrogen, Carlsbad, Calif.; Pharmingen, San Diego, Calif.), etc. The insertion info a cloning vector can, for example, be accomplished by ligating the DNA fragment into a cloning vector which has complementary cohesive termini, by blunt end ligation if no complementary cohesive termini are available or by through nucleotide linkers using techniques standard in the art. E.g., Ausubel et al. (eds.), Current Protocols in Molecular Biology, (1992). Recombinant vectors comprising the nucleic acid of interest may then be introduced into a host cell compatible with the vector (e.g. E. coli, insect cells, mammalian cells, etc.) via transformation, transfection, infection, electroporation, etc. The nucleic acid may also be placed in a shuttle vector which may be cloned and propagated to large quantities in bacteria and then introduced into a eukaryotic cell host for expression. The vector systems of the present invention may provide expression control sequences and may allow for the expression of proteins in vitro.

Natural, recombinant or synthetic carnitine acyltransferase may be purified by methods known in the art, including, but not limited to, selective precipitation, dialysis, chromatography, and/or electrophoresis. Purification may be monitored by measuring the ability of a fraction to perform acyl transfer of an appropriate substrate, and then calculating the specific activity. Any standard method of measuring acyl transferase activity may be used.

For certain embodiments, it may be desirable to express a carnitine acyltransferase as a fusion protein. In specific non-limiting embodiments, said fusion protein comprises a tag which facilitates purification. As referred to herein, a “tag” is any added series of amino acids which are provided in a protein at either the C-terminus, the N-terminus, or internally. Suitable tags include but are not limited to tags known to those skilled in the art to be useful in purification such as, but not limited to, His tag, glutathione-s-transferase tag, flag tag, mbp (maltose binding protein) tag, etc. Such tagged proteins may also be engineered to comprise a cleavage site, such as a thrombin, enterokinase or factor X cleavage site, for ease of removal of the tag before, during or after purification. Vector systems which provide a tag and a cleavage site for removal of the tag are particularly useful to make the expression constructs of the present invention. A tagged carnitine acyltransferase may be purified by immuno-affinity or conventional chromatography, including but not limited to, chromatography employing the following: glutathione-Sepharose™ (Amersham-Pharmacia, Piscataway, N.J.) or an equivalent resin, nickel or cobalt-purification resins, preferably nickle-agarose resin, anion exchange chromatography, cation exchange chromatography, hydrophobic resins, gel filtration, antiflag epitope resin, reverse phase chromatography, etc.

After purification, preferably such that at least 70 percent, and more preferably at least 90 percent, of total protein is carnitine acyltransferase, the enzyme, or a mixture of the enzyme and one or more substrate thereof, may be concentrated to greater than 1 mg/ml for crystallization purposes. In a preferred embodiment, the concentration is greater than 10 mg/ml and in a particularly preferred embodiment, the concentration is greater than 20 mg/l.

Any crystallization technique known to those skilled in the art may be employed to obtain the crystals of the present invention, including, but not limited to, batch crystallization, vapor diffusion (either by sitting drop or hanging drop) and micro dialysis. Seeding of the crystals in some instances may be required to obtain X-ray quality crystals. Standard micro and/or macro seeding of crystals may therefore be used. In a preferred embodiment, the crystals are obtained using the sitting-drop vapor diffusion method.

To collect diffraction data from the crystals of the present invention, the crystals may be flash-frozen in the crystallization buffer employed for the growth of said crystals, however with preferably higher precipitant concentration (see, Examples below). For example, but not by way of limitation, if the precipitant used was 20% PEG 3350, the crystals may be flash frozen in the same crystallization solution employed for the crystal growth wherein the concentration of the precipitant is increased to 25% (see Examples below). If the precipitant is not a sufficient cryoprotectant (i.e. a glass is not formed upon flash-freezing), cryoprotectants (e.g. glycerol, low molecular weight PEGs, alcohols, etc) may be added to the solution in order to achieve glass formation—upon flash-freezing, providing the cryoprotectant is compatible with preserving the integrity of the crystals. The flash-frozen crystals are maintained at a temperature of less than −110° C. and preferably less than −150° C. during the collection of the crystallographic data by X-ray diffraction.

Preferably, the protein crystals and protein-substrate complex co-crystals of the present invention diffract to a high resolution limit of at least equal to or greater than 3 angstrom (Å); it should be noted that a greater resolution is associated with the ability to distinguish atoms placed closer together. In a more preferred embodiment, the protein crystals and protein-substrate complex co-crystals of the present invention diffract to a high resolution limit of greater than 2.5 Å.

A crystal of the present invention may take a variety of forms. In a preferred embodiment, the crystal has a space group of C2 with two molecules in the asymmetric unit and with unit dimensions for the free enzyme of a=158.9 Å, b=89.6 Å, c=119.4 Å and β=127.5° (see, e.g., Examples, below), for the carnitine complex, of a=160.7 Å, b=91.7 Å, c=122.9 Åand β=129.00 and for the CoA complex, a=162.3 Å, b=92.0 Å, c=122.9 Åand β=129.0°.

Data obtained from the diffraction pattern may be solved directly or may be solved by comparing it to a known structure, for example, the three dimensional structure of mCRAT (with or without substrates) or hCRAT. If the crystals are in a different space group than the known structure, molecular replacement may be employed to solve the structure, or if the crystals are in the same space group, refinement and difference fourier methods may be employed. The structure of carnitine acyltransferases, as defined herein, exhibit no greater than a 4.0 Å, preferably no greater than a 2.0 Å root mean square deviation (RMSD) in the positions of the Cα atoms for at least 50% or more of the amino acids. In a particularly preferred embodiment, the structure of carnitine acyltransferases, as defined herein, exhibit no greater than a 0.5 Å, RMSD in the positions of the Ca atoms for at least 50% or more of the amino acids.

In a preferred specific, non-limiting embodiment of the present invention, seleno-methionyl proteins may be used to directly determine the structure of a carnitine acyltransferase. (Hendrickson, W. A., 1991, Science 254(5028):51-58). For example, a seleno-methionyl single wavelength anomalous diffraction (SAD) data set may be collected at 1000K on the free enzyme and native reflection data sets may be collected for enzyme/substrate complexes. X-ray diffraction data may be processed with the HKL package (Otwinowski and Minor, 1997, Methods Enzymol. 276:307-326). The location of seleno-methionyl atoms may be determined with the program SnBv2.0 (Weeks and Miller, 1999, Acta Crystallogr D Biol Crystallogr 55(2):492-500) and may further be confirmed with SHELXS (Sheldrick, 1990, Acta Crystal. A46:467-473). Reflection phases, preferably to less than or equal to 4.0 Å may be calculated based on the SAD data and may further be improved with the program SOLVE (Terwilliger and Berendzen, 1999, Acta Cryst. D55:849-861). The resulting atomic model may be fed into the electron density with the problem O (Jones et al., 1991, Acta Crystal. A47:110-119). A structure of the enzyme/substrate complex may be determined by molecular replacement with the program COMO (Jogl et al., 2001, COMO: A program for combined molecular replacement, ACTA Cryst. D57: 1127-1134) and structural refinement may be carried out with the program CNS (Brunger et al., 1998, Acta Cryst. D54:905-921).

In addition, programs such as DENZO, SCALEPACK and HKL (Otwinowski & Minor, 1997, Method Enzymol. 276:307-326) may be employed in the determination of the three-dimensional structure. Any method known to those skilled in the art may be used to process the X-ray diffraction data. In addition, in order to determine the atomic structure of a carnitine acyltransferase according to the present invention, multiple isomorphous replacement (MIR) analysis, model building and refinement may be performed. For MIR analysis, the crystals may be soaked in heavy-atoms to produce heavy atom derivatives necessary for MIR analysis. As used herein, heavy atom derivative or derivatization refers to the method of producing a chemically modified form of a protein or protein complex crystal wherein said protein is specifically bound to a heavy atom within the crystal. In practice a crystal is soaked in a solution containing heavy metal atoms or salts, or organometallic compounds, e.g., lead chloride, gold cyanide, thimerosal, lead acetate, uranyl acetate, mercury chloride, gold chloride, etc, which can diffuse through the crystal and bind specifically to the protein. The location(s) of the bound heavy metal atom(s) or salts can be determined by X-ray diffraction analysis of the soaked crystal. This information is used to generate MIR phase information which is used to construct the three-dimensional structure of the crystallized CRAT and CRAT-related proteins of the present invention. Thereafter, an initial model of the three-dimensional structure may be built using the program O (Jones et al., 1991, Acta Crystallogr. A 47:110-119). The interpretation and building of the structure may be further facilitated by use of the program CNS (Brunger et al., 1998, Acta Crystallogr. D 54:905-921).

The term “molecular replacement” broadly refers to a method that involves generating a preliminary model of the three-dimensional structure of a carnitine acyltransferase crystal of the present invention whose structural coordinates were previously unknown. Molecular replacement is achieved by orienting and positioning a molecule whose structural coordinates are known (e.g., mCRAT or hCRAT, as described herein) within the unit cell as defined by the X-ray diffraction pattern obtained from the carnitine acyltransferase under study (or the corresponding enzyme/substrate complex) so as to best account for the observed diffraction pattern of the unknown crystal. Phases can then be calculated from this model and combined with the observed amplitudes to give an approximate Fourier synthesis of the structure whose coordinates are unknown. This in turn can be subject to any of several forms of refinement to provide a final, accurate structure.

The molecular replacement method may be applied using techniques known to the skilled artisan. For example, the program AMORE (1994, “The CCP4 suite: Programs for computational crystallography”, Acta Crystallogr. D50:760-763) may be employed to determine the previously unknown structure of a carnitine acyltransferase or its enzyme/substrate complex by molecular replacement using the mCRAT and hCRAT coordinates as set forth herein as FIGS. 7 and 10, respectively, as reference structures.

The three-dimensional structural information and the specific atomic coordinates associated with said structural information of CRAT are useful for solving the structure of crystallized forms of other carnitine acyltransferases This technique may also be applied to solve the structures of CRAT-related proteins outside of the carnitine acyltransferase family. Such CRAT-related proteins comprise a root mean square deviation (RMSD) of no greater than 4.0 Å, and preferably no greater than 2.0 Å in the positions of Coc atoms for at least 50 percent or more of the amino acids of the structure of the CRAT of the present invention. Such an RMSD may be expected based on the amino acid sequence identity (Chothia and Lesk, 1986, Embo J. 5:823-826).

The refined three-dimensional CRAT structures of the present invention, specifically mCRAT, mCRAT:carnitine complex, mCRAT:CoA complex and hCRAT are represented by the atomic coordinates set forth in FIGS. 7-10 respectively. A description of various specific features of these structures is presented in the following paragraphs. Such description may be useful for solving the structures of other carnitine acyltransferases.

The crystal structure of the free enzyme of mCRAT has been determined at 1.8 Å resolution. The current atomic model contains residues 30-625 for each of the two molecules in the asymmetric unit, which essentially corresponds to the full-length, mature form of this enzyme. The information obtained from the three-dimensional structures of the present invention reveals that the structure of CRAT contains 16 β-strands (β1-β16) and 20 α-helices (α1-α20), and can be divided into two domains as shown in FIG. 2A. FIG. 2B shows that the C domain contains a six-stranded mixed β-sheet, together with eleven α-helices, and that residues in this domain include the C-terminal third of the protein (residues 407-626) as well as residues 30-95 at the N-terminus, which contribute four α-helices to this domain and interact with an inserted segment between α17 and β13. One face of the β-sheet in this domain is covered by the α-helices, whereas part of the other face of this sheet contacts the N domain of the structure (residues 96-385). FIG. 2A further shows that Helix α3, with about 20 residues, forms the long connection between the N and the C domains.

FIG. 2C shows that the N domain contains an eight-stranded mixed β-sheet, which is covered on both sides by eight α-helices. This eight-stranded β-sheet is formed by flanking the central six-stranded sheet with one extra β-strand on each of its edges. FIG. 2B shows that one of these extra β-strands actually comes from the C domain. Part of the β13-β15 cross-over connection in the C domain forms a β-strand (β14), and this strand is hydrogen-bonded to β1 in an anti-parallel fashion in the N domain as shown in FIG. 2C. The N domain also contains a pair of β-strands (β2 and β3) on the surface, helping enclose one end of the β-sheet.

FIGS. 2B and 2C show that the N and C domains share very similar polypeptide backbone folds, despite the lack of any recognizable amino acid sequence homology between them. This structural homology is limited to the core of the two domains, including the central six β-strands of the β-sheet and three α-helices closely-associated with it (α6, α7 and α12 in the N domain). A total of 71 Cα positions can be superimposed to within 3.5 Å of each other between the two domains, and the rms distance for these equivalent atoms is 2.0 Å. However, there are only three pairs of identical residues (4%) among these structurally-aligned positions, underscoring the lack of amino acid sequence conservation between the two domains.

Comparisons with the protein structure database, performed with the program Dali (Holm and Sander, 1993, J. Mol Biol 233:123-138), revealed that the N and the C domains of mCRAT have similar backbone folds as that of chloramphenicol acetyltransferase (CAT) as depicted in FIG. 2D (Leslie et al., 1988, Proc Natl Acad Sci USA 85:4133-4137) and the catalytic domain of dihydrolipoyl transacetylase (E2 pCD) (Mattevi et al., 1992, Science 255:1544-1550). Both of these enzymes catalyze the transfer an acetyl group from acetyl-CoA to an organic substrate. In addition, E2p forms the cubic core of the pyruvate dehydrogenase multienzyme complex (Mattevi et al., 1992, Science 255:1544-1550). A total of 161 out of 213 Cα atoms in CAT can be superimposed with the equivalent atoms in the C domain of CRAT, giving a rms of 3.7 Å. The amino acid sequence identity among these structurally equivalent residues is only 9 percent. Similar observations are made when the CAT structure is aligned with the N domain of CRAT, or when the structure of E2 pCD is used for comparison.

The structural conservation between CRAT and CAT or E2 pCD extends beyond the similarity in their backbone folds. The organization of the two domains of CRAT is similar to that of two monomers in the trimers of CAT and E2 pCD (FIG. 2E). The rotational relationship between the N and C domains of CRAT is 118°, close to a three-fold rotation. Both CAT and E2 pCD function as trimers, and the active sites of the two enzymes are each located at the interface between a pair of monomers in the trimer (FIG. 2E) (Leslie et al., 1988, Proc Natl Acad Sci USA 85:4133-4137; Mattevi et al., 1992, Science 255:1544-1550). It appears that CRAT may have evolved by gene duplication of a single-domain enzyme (such as E2p or CAT), and the resulting two-domain enzyme has the same function as a homo-timer of the single-domain enzymes.

The amino acid sequences of CRAT have diverged significantly from those of CAT and E2 pCD. In addition, the N and C domains of CRAT contain about 300 amino acid residues each, larger than the sizes of CAT and E2 pCD (about 220 residues). As a result, the two domains in CRAT have significant insertions in several of the surface loops as compared to CAT or E2 pCD (FIGS. 2B-D). These insertions form additional interactions within the domains, for example those between the α18-α19 insertion and helices α2-α4 in the C domain (FIG. 2B). In the N domain, the α8-α10 and α11 insertions cover the face of the β-sheet (FIG. 2C) that would be shielded from solvent by the third monomer in the trimers of CAT and E2 pCD.

Previous biochemical, kinetic, and mutagenesis studies have identified a histidine residue, equivalent to His343 in mCRAT (FIG. 1), as the catalytic residue for the carnitine acyltransferases (McGarry and Brown, 1997, Eur. J. Biochem. 244:1-14; Ramsay et al., 2001, Biochim. Biophys. Acta 1546:21-43). Referring to FIG. 2C, the structural analysis of the present invention confirms the functional importance of the His343 residue, in the connection between β8 and α12 in the N domain. As shown in FIG. 2D, it is located at the same position as His195 in CAT and His610 in E2 pCD, the catalytic residues of these enzymes (Leslie et al., 1988, Proc. Natl. Acad. Sci. U.S.A. 85:4133-4137; Mattevi et al., 1992, Science 255:1544-1550). As in CAT and E2 pCD, the His343 residue in CRAT is held in an unusual conformation, such that the Nδ1 ring nitrogen in the side chain is hydrogen-bonded to the carbonyl oxygen in its main chain.

FIG. 2A shows that the active site of mCRAT is located deep in the enzyme, at the interface between the N and C domains. The His343 residue can be reached by two separate channels, of 15-18 Å depth each, from opposite sides of the protein (FIG. 2A). One of these channels is used for the binding of the CoA substrate, whereas the other is for binding carnitine. While the overall arrangement of the active site is similar to that in CAT (FIG. 2E) and E2 pCD (Leslie et al., 1988, Proc. Natl. Acad. Sci. U.S.A. 85:4133-4137; Mattevi et al., 1992, Science 255:1544-1550), there are significant differences in the binding modes of the substrates, especially CoA, as well as the catalytic mechanism, between mCRAT and these other enzymes (see below).

The crystal structure of mCRAT in complex with the substrate carnitine has been determined at 1.9 Å resolution (Table 1 below). The binding mode of carnitine is clearly defined by the crystallographic analysis (FIG. 3A). X-ray diffraction data on crystals of mCRAT grown in the presence acetylcarnitine, the product of the reaction, has also been collected. The subsequent crystallographic analysis, at 1.9 Å resolution, showed however the presence of only carnitine in the active site. This suggests that the acetylcarnitine has been hydrolyzed to carnitine during the crystallization process, and this hydrolysis may possibly be catalyzed by the enzyme itself.

Carnitine is bound in a partially-folded conformation, with its carboxyl group pointed in the opposite direction from the hydroxyl group (FIG. 3A). This bound conformation corresponds to one of the favored rotamers of this compound in solution, as indicated by NMR studies of free carnitine in aqueous solution (Colucci et al., 1986, J. Amen. Chem. Soc. 108:7141-7147).

The carnitine binding site is formed by the β-sheet (strands β11-β14) in the C domain, and residues in α5-β1 and β8-α12 in the N domain (FIG. 3B). One face of the carnitine molecule is exposed to the solvent in the bound state (FIG. 3C). The 3-hydroxyl group on carnitine forms a hydrogen-bond with the side chain Nε2 atom of the catalytic His343 residue (FIG. 3D), which has implications for the catalytic mechanism of this family of enzymes. The structural analysis demonstrates that the catalysis is stereo-specific, as the other stereo-isomer of the hydroxyl group cannot maintain the same interactions with the enzyme.

The carboxylate group of carnitine has electrostatic interactions with the side chain guanidinium group of Arg518 (from helix α18), with a distance of about 4 Å, as well as a network of hydrogen-bonding interactions (FIGS. 3B and 3D). One of the carboxylic oxygen atoms is hydrogen-bonded to the side chain hydroxyls of Tyr452 and Ser454 (β11), whereas the other is hydrogen-bonded to the side chain hydroxyl of Thr465 (β12) and a water molecule. The tetrahedral coordination of this water is completed by three ligands from the enzyme, the Nε1 atom of Trp102 (α5), the Oil atom of Tyr107 (α5) and the Oε2 atom of Glu347 (β8-α12 loop), which also shield it from the solvent (FIG. 3B).

The trimethylammonium group of carnitine is situated over the aromatic ring of the side chain of Phe566 (β14), with a separation of about 3.6 Å (FIG. 3B). It is also close to the side-chain of Ser552 (β13) and Val569 (β14-β15 loop). However, there are no negatively-charged residues in the immediate vicinity of this group to balance its positive charge. Our structural analysis suggests this positive charge may be important for the catalytic activity of the enzyme (see below).

CRAT has a pre-formed binding site for the substrate carnitine. The only significant conformational difference in the active site between the free enzyme and the carnitine complex is in the side chain of Ser454, which adopts a different rotamer to have better hydrogen-bonding interactions with the carboxylate of carnitine (FIG. 3D). Outside the active site, conformational differences are observed for several regions on the surface of the structure. In addition, there are small rigid-body movements of the domains relative to each other. However, these changes are more likely due to flexibility in the enzyme or differences in crystal packing, and they have only minor impact on the active site region.

The crystal structure of CRAT in complex with the substrate CoA has been determined at 2.3 Å resolution (Table 1). To obtain this structure, X-ray diffraction data were collected on a crystal of the free enzyme of CRAT that was soaked overnight with 1.5 mM acetyl-CoA. The crystal suffered serious damage during the soak, giving rise to higher R factors for the diffraction data and the atomic model (Table 1). Nonetheless, the data set obtained was of sufficient quality to clearly define the binding mode of the substrate (FIG. 4A). It also indicates that the compound bound in the active site is actually CoA (FIG. 4A). This suggests that acetyl-CoA has been hydrolyzed to CoA during the overnight soaking process. Therefore, it appears that CRAT may be able to catalyze the hydrolysis of both acetylcarnitine and acetyl-CoA.

The CoA binding site is on the opposite side of the His343 side chain from the carnitine binding site (FIG. 2A). A unique feature of the β-sheet in the C domain is that the neighboring parallel strands β11 and β13 are splayed apart from each other at the C-terminal end (FIG. 4B). This creates an opening between the two strands, and allows the pantothenic arm of CoA to thread through it to reach the active site (FIG. 4B). In the active site, the thiol group forms a hydrogen-bond with the side chain Nε2 atom of the catalytic His343 residue.

The CoA molecule is bound in the fully-extended, linear conformation (FIG. 4B). The adenine base at one end of the molecule is about 25 Å from the thiol group at the other. This binding mode is in sharp contrast to the binding of CoA to CAT and E2 pCD, where a folded conformation of CoA is observed (Leslie et al., 1988, Proc. Natl. Acad. Sci. U.S.A. 85:4133-4137; Mattevi et al., 1992, Science 255:1544-1550). A comparison of the two binding modes-showed that the pantotheine portion has similar conformations, but the ADP portion has large differences (FIG. 4C). The adenine bases are separated by about 14 Å in the two binding conformations. In CAT and E2 pCD, the N1 and N6 atoms of the adenine base are hydrogen-bonded to the main chain amide and carbonyl of residues near the end of strand βI (FIG. 2E). In mCRAT, the equivalent residue, at the end of strand β15, is Pro580 (FIG. 4B), which would abolish the type of interactions observed in CAT and E2 pCD as it not only eliminates the hydrogen-bond but also introduces steric clash with the adenine base. In the structure of the present invention, the adenine base lies on the surface of the enzyme (FIGS. 5B and 5D) and there appears to be no specific recognition of its N1 or N6 atoms.

Residues in the CoA binding pocket are generally conserved among the carnitine acyltransferases (FIG. 1). These include Lys419 and Lys423, which recognize the 3′-phosphate group of CoA (FIG. 4B). In the binding site for the pantothenic arm of CoA, conserved residues Asp430 and Glu453 are directly hydrogen-bonded to each other FIG. 4B). Mutation of either residue can reduce the activity of the enzyme (Ramsay et al., 2001, Biochim. Biophys. Acta. 1546:21-43).

The structure of the substrate complexes of mCRAT of the present invention also helps to reveal the possible binding site for the hydrocarbon groups of long-chain fatty acids in the CPTs. The acetyl group of acetylcarnitine, modeled based on the structure of the carnitine complex, points towards a hydrophobic pocket that is enclosed by the intersection of the two β-sheets in the enzyme (strands β1 and β8 in the N domain, strands β13 and β14 in the C domain) and helix α12 (FIG. 5). In mCRAT, this pocket is partly filled by the side chain of Met564, coming from strand β14 (FIG. 5). The equivalent residue in the CPTs is a glycine (FIG. 1), which should make it possible for the hydrocarbon groups of the long-chain fatty acid to bind in this pocket. The mutation of this Gly to Glu can cause L-CPT-I deficiency in patients (Ramsay et al., 2001, Biochim. Biophys. Acta 1546:21-43). Additional differences in the amino acid sequences of mCRAT and CPTs, for example in the α5-β1 region (FIGS. 1 and 3B), may also contribute to substrate binding in the CPTs.

The structures of the free enzyme and the substrate complexes of mCRAT of the present invention convincingly demonstrate the catalytic mechanism of this family of enzymes (FIG. 6). The His residue in the active site acts as a general base in the catalysis (McGarry and Brown, 1997, Eur. J. Biochem. 244:1-14; Ramsay et al., 2001, Biochim. Biophys. Acta 1546:21-43). It extracts the proton from the 3-hydroxyl group of carnitine or the thiol group of CoA, depending on the direction of the reaction. In the structures of the carnitine and CoA complexes disclosed herein, the reactive groups of both substrates are directly hydrogen-bonded to the His343 side chain, so that the substrates are optimally positioned in the disclosed structures for the catalysis to occur. The activated hydroxyl or thiol group can then directly attack the carbonyl carbon in acyl-CoA or acylcarnitine, and the reaction proceeds without the formation of an acyl-enzyme intermediate.

It is also clear from the mechanism and the disclosed structures that if only acylcarnitine or acyl-CoA is bound in the active site, a water molecule binds in the opposite channel of the active site and can become the receptor for the acyl groups. Therefore, these transferases can also catalyze the simple hydrolysis of acylcarnitine or acyl-CoA, as we observed in our crystallization experiments.

Structural analysis and modeling studies suggest that the trimethylammonium group of carnitine may play an important role in the catalysis by these acyltransferases, by stabilizing the oxyanion in the tetrahedral intermediate of the reaction (FIG. 6). This suggests that carnitine acyltransferases may be an example of substrate-assisted catalysis (Dall'Acaqua and Carter, 2000, Protein Sci. 9:1-9). The structure described herein for the intermediate places the oxyanion at about 3 Å distance to the trimethylammonium group of carnitine, suggesting strong, favorable interactions between the two charges (FIG. 6). This is supported by the observation that the positive charge of carnitine is not critical for its binding, but is absolutely required for catalysis (Saeed et al., 1993, Arch. Biochem. Biophys. 305:307-312). A carnitine analog, replacing the trimethylammonium group with the neutral t-butyl group, is not a substrate of the enzyme but can compete with carnitine for binding to the active site.

Based on the description for the tetrahedral intermediate of the present invention, the oxyanion is also within 3 Å of the side chain hydroxyl of Ser554 (FIG. 6). Mutation of this Ser residue, in the STS motif described earlier, produced a 10-fold decrease in the k_{cat} while having little impact on the Kₘ for carnitine (Cronin, 1997, Biochem. Biophys. Res. Commun. 238:784-789). The equivalent Ser residue in CAT and E2 pCD has also been identified as the oxyanion hole of those enzymes (Hendle et al., 1995, Biochem. 34:4287-4298; Lewendon et al., 1990, Biochem. 29:2075-2070). However, the small effect of the S554A mutation on the catalysis by COT also supports the functional role of the carnitine substrate itself in stabilizing the transition-state of this reaction.

### Design of Modulators

Modulators of carnitine acyltransferases may be designed, according to the invention, using three-dimensional structural information obtained as set forth in the preceding section. This structural information may be used to build molecules which are able to form the desired interactions with one or more substrate binding site and/or active site (i.e., reactive site) of the carnitine acyltransferase.

Where the carnitine acyltransferase is not CRAT, and its structure has been solved as described above with reference to the CRAT crystal structure, a model of the reactive site may be developed using methods as set forth in this section. The resulting model may be used directly to design, select, or identify modulators of the “non-CRAT” carnitine acyltransferase.

Alternatively, the CRAT reactive site model described herein may be used to either directly develop a modulator for CRAT or indirectly develop a modulator of a “non-CRAT” carnitine acyltransferase for which the structure has not yet been solved. A modulator designed to interact with a CRAT reactive site may be reasonably expected to interact not only with CRAT enzyme but also with the reactive sites of other carnitine acyltransferases due to the strong structural homology shared by members of this enzyme family (even where amino acid homology is not great). The ability for such a modulator to modulate the activity of a non-CRAT carnitine acyltransferase is desirably confirmed by further computer analysis, in vitro and/or in vivo testing.

In non-limiting embodiments, the present invention provides for a model, actual or virtual, of a CRAT reactive site that comprises the catalytic histidine residue (e.g., His343 of mCRAT, His341 of hCRAT) and two substrate-binding sites, preferably comprised in separate channels of 15-18 Å depth each extending in approximately opposite directions from the His residue. One substrate-binding site is capable of binding carnitine, the other is capable of binding CoA. The three dimensional orientation of atoms in the catalytic histidine residue, the substrate binding sites, and the two channels is as set forth by the atomic coordinates for these elements provided for mCRAT and hCRAT herein, or within 4 angstroms thereof.

The reactive site model may be comprised in a virtual or actual protein structure that is smaller than, larger than, or the same size as a native CRAT protein. The protein environment surrounding the reactive site model may be homologous or identical to native CRAT, or it may be partially or completely non-homologous.

In particular non-limiting embodiments of the invention, the carnitine binding site of the model reactive site is as schematically depicted in FIG. 3, in particular in the regions of β-sheet (strands β11-β14) in the C domain, and regions α5-β1 and β8-α12 in the N domain. In another set of specific, non-limiting embodiments, carnitine binding site comprises amino acid residues TRP102, LEU103, ALA106, TYR107, ILE116, TYR117, SER118, SER119, PRO120, GLY121, TYR341, GLU342, HIS343, ALA344, ALA345, ALA346, GLU347, GLY348, PRO349, PRO350, ILE351, TYR452, GLU453, SER454, ALA455, SER456, LEU457, ARG458, ARG464, THR465, ASP466, THR467, ILE468, ARG469, ARG518, LEU551, SER552, THR553, SER554, GLN555, VAL556, MET564, PHE565, PHE566, GLY567, PRO568, and VAL569 and preferably Arg518, Thr465, Trp102, Tyr107, Glu347, His343, Phe566, Val569, Ser552, Ser454, and Tyr452 (see FIG. 3D), in a configuration as defined by the atomic coordinates set forth in FIG. 7 for unbound mCRAT or in a configuration as defined by the coordinates set forth in FIG. 8 for mCRAT bound to carnitine. In yet another set of specific, non-limiting embodiments, the carnitine binding site comprises TRP102, LEU1.03, ALA106, TYR107, ILE116, TYR117, SER118, SER119, PRO120, GLY121, TYR341, GLU342, HIS343, ALA344, ALA345, ALA346, GLU347, GLY348, PHE349, PRO350, ILE351, TYR452, GLU453, SER454, ALA455, SER456, LEU457, ARG458, ARG464, THR465, ASP466, THR467, ILE468, ARG469, ARG518, LEU551, SER552, THR553, SER554, GLN555, VAL556, MET564, PHE565, PHE566, GLY567, PRO568, VAL569 and preferably Arg516, Thr463, Trp100, Tyr105, Glu345, His341, Phe564, Val567, Ser550, Ser452 and Tyr450 in a configuration as defined by the atomic coordinates set forth in FIG. 10A for hCRAT bound to carnitine, to a variation of within 4 angstroms.

In particular non-limiting embodiments of the invention, the CoA binding site of the model reactive site is as schematically depicted in FIG. 4. In another set of specific, non-limiting embodiments, the CoA binding site comprises amino acid residues LEU163, LEU168, HIS343, GLU347, GLY348, PRO349, PRO350, LYS419, ASP420, PHE421, PRO422, LYS423, LEU427, SER428, PRO429, ASP430, ALA431, PHE432, ILE433, GLN434, TYR452, GLU453, SER454, ALA455, SER456, LEU457, ARG458, MET459, ARG464, THR465, ASP466, THR467, ILE468, VAL500, GLN501, HIS503, ARG504, THR507, ASP508, ILE511, GLN555, PRO557, TYR578, PRO580, MET581, and GLU582 and preferably Lys419, Lys423, Asp430 and Glu453, having coordinates as set forth in FIG. 7 for unbound mCRAT and in FIG. 9 for mCRAT bound to CoA, to a variation of within 4 angstroms. In yet another set of specific, non-limiting embodiments, the CoA binding site comprises LEU163, LEU168, HIS343, GLU347, GLY348, PHE349, PRO350, LYS419, ASP420, PHE421, PRO422, LYS423, LEU427, SER428, PRO429, ASP430, ALA431, PHE432, ILE433, GLN434, TYR452, GLU453, SER454, ALA455, SER456, LEU457, ARG458, MET459, ARG464, THR465, ASP466, THR467, ILE468, VAL500, GLN501, HIS503, ARG504, THR507, ASP508, ILE511, GLN555, PRO557, TYR578, PRO580, MET581, and GLU582 and preferably Lys417, Lys221, Asp428 and Glu451 in a configuration as defined by the atomic coordinates set forth in FIG. 10B for hCRAT bound to CoA, to a variation of within 4 angstroms.

Thus, the present invention provides for a method for rationally designing a modulator of a carnitine acyltransferase, comprising the steps of (i) producing a computer readable model of a molecule comprising a carnitine acetyltransferase (e.g. CRAT) reactive site; and (ii) using the model to design a test compound having a structure and a charge distribution compatible with (i.e., able to be accommodated within) the reactive site, wherein the test compound comprises a functional group that may interact with the reactive site to modulate carnitine acyltransferase activity.

The atomic coordinates of atoms in the reactive site of CRAT or another carnitine acyltransferase may be used in conjunction with computer modeling using a docking program such as GRAM, DOCK, HOOK or AUTODOCK (Dunbrack et al., 1997, Folding &Design 2:27-42) to identify potential modulators. This procedure can include computer fitting of potential modulators to a reactive site model to ascertain how well the shape and the chemical structure of the potential modulator will complement the active site or to compare the potential modulators with the binding of substrate or known inhibitor molecules in the active site.

Computer programs may be employed to estimate the attraction, repulsion and/or stearic hindrance associated with a postulated interaction between the reactive site model and the potential modulator compound. Generally, characteristics of an interaction that are associated with modulator activity include, but are not limited to, tight fit, low stearic hindrance, positive attractive forces, and specificity.

Modulator compounds of the present invention may also be designed by visually inspecting the three-dimensional structure of the reactive site, CRAT or other carnitine acyltransferase, a technique known in the art as “manual” drug design. Manual drug design may employ visual inspection and analysis using a graphics visualization program known in the art.

In designing potential modulator compounds according to the invention, the functional aspect of a modulator may be directed at a particular step of the CRAT catalytic mechanism, as illustrated by the following non-limiting examples.

A first example is based on a reaction mechanism where, in the natural function of CRAT, the catalytic His residue extracts a proton from the 3-hydroxyl group of carnitine or the thiol group of CoA, depending on the direction of the reaction. According to the invention, a modulator intended to decrease acetyl-CoA levels (by removing the acetyl group) may be designed as a carnitine mimic which enhances the likelihood of proton extraction from a functional group on the modulator corresponding to the 3-hydroxyl of carnitine, hence facilitating acetylation of the modulator.

In a second example, in view of the observation that if only acetylcarnitine or acetyl-CoA is bound in the reactive site, a water molecule binds in the opposite channel and can become the receptor for the acetyl group, resulting in simple hydrolysis of acetylcarnitine or acetyl-CoA, a modulator that promotes hydrolysis of one of these substrates may be designed to block the other substrate's access to its binding site. For example, a modulator that promotes hydrolysis of acetyl-CoA may be designed to block binding of carnitine (but permit binding of a water molecule) to its substrate binding site.

A third example is based on observations that suggest that the trimethylammonium group of carnitine may play an important catalytic role by stabilizing the oxyanion in the tetrahedral intermediate of the reaction. A modulator intended to facilitate catalysis may be designed to further stabilize the oxyanion, for example by providing a functional group with a greater positive charge than the trimethylammonium group. A modulator intended to inhibit catalysis may be designed to destabilize the oxyanion, and may carry a substituent for the trimethylammonium group, for example, a group with a lesser positive charge.

A fourth example is based on the observation that the side chain of Ser454 changes conformation upon binding to carnitine to facilitate hydrogen-binding interactions with the carboxylate of carnitine. A modulator intended to act as a carnitine antagonist may be designed to obstruct this conformational change.

A fifth example is based on the observation that, according to the CRAT structure provided herein, the acetyl group of acetylcarnitine, modeled based on the structure of the carnitine complex, points towards a hydrophobic pocket that is enclosed by the intersection of the two β-sheets in the enzyme (strands β1 and β18 in the N domain, strands β13 and β14 in the C domain) and helix α12 (FIG. 5). In CRAT, this pocket is partly filled by the side chain of Met564, coming from strand β14 (FIG. 5). The equivalent residue in the CPTs is a glycine (FIG. 1), which should make it possible for the hydrocarbon groups of the long-chain fatty acid to bind in this pocket. A modulator of CPT activity may be designed which inhibits the ability of a long-chain fatty acid to bind in this pocket.

### Screening for Modulator Compounds

As an alternative or an adjunct to rationally designing modulators, random screening of a small molecule library for compounds that interact with and/or bind to a reactive site of CRAT or another carnitine acyltransferase may be used to identify useful compounds. Such screening may be virtual; small molecule data bases can be computationally screened for chemical entities or compounds that can bind to or otherwise interact with a virtual model of a CRAT reactive site. Alternatively, screening can be against actual molecular models of the reactive site.

### Assay Systems

Potential modulators of carnitine acyltransferase activity, produced, for example, by rational drug design or by screening of libraries as described above, may be subjected to one of the following assays to confirm their activity.

A potential modulator may be evaluated for its ability to physically interact with CRAT or another carnitine acyltransferase by co-crystallizing the potential modulator with CRAT or another carnitine acyltransferase and then determining the structure of the resulting co-crystal. For example, the structure of the co-crystal may be determined by molecular replacement to assess the binding characteristics. The ability of the compound to modulate enzyme activity may be correlated with its ability to physically interact with the reactive site and/or to assume an orientation that would facilitate or inhibit acyl exchange (as discussed above). In one specific example, a modulator may be determined to interact with a mCRAT reactive site if, in a co-crystal of the modulator and mCRAT, the modulator contains an atom, and preferably a functional group, within 10 angstroms of an atom comprised in one or more of the following mCRAT amino acid residues: Arg518, Thr465, Trp102, Tyr107, Glu347, His343, Phe566, Val569, Ser552, Ser454, Tyr452, Lys419, Lys423, Asp430 and Glu453. In another specific example, a modulator may be determined to interact with a hCRAT reactive site if, in a co-crystal of the modulator and mCRAT, the modulator contains an atom, and preferably a functional group, within 10 angstroms of an atom comprised in one or more of the following hCRAT amino acid residues: Arg516, Thr463, Trp100, Tyr105, Glu345, His341, Phe564, Val567, Ser550, Ser452, Tyr450, Lys417, Lys421, Asp428 and Glu451.

The present invention further provides for assays comprising incubating the potential modulator with a carnitine acyltransferase, such as CRAT, performing gel filtration to separate any free potential modulator from CRAT-bound modulator, and determining the amount of acyl exchange activity of the modulator-bound enzyme. To measure binding constants (e.g., Kd), methods known to those in the art may be employed such as Biacore™ analysis, isothermal titration calorimetry, Elisa with substrate on the plate to show competitive binding, or by a acyl exchange activity assay. Similarly, the reaction rate may be measured by methods known in the art.

The present invention further provides for methods that determine the effect of a potential modulator iv vivo. Such methods may provide important information, as the effect of the modulator on molecules involved in interrelated pathways may be determined. For example, a potential modulator may be administered to a cell that is capable of performing fatty acid oxidation, such as, but not limited to, a liver, a heart cell, or a skeletal cell (in the form of a mature liver, heart or skeletal cell or a transformed liver. heart or skeletal cell), and then the level of one or more molecules involved in fatty oxidation, the Embden-Meyerhoff pathway, the Krebs cycle, mitochondrial electron transport, fatty acid synthesis, and gluconeogenesis, including insulin, glycogen, cholesterol, and ketone bodies, may be measured, and the success or failure of the potential modulator to achieve the desired effect may be determined. For example, but not by way of limitation, a modulator intended to be used as a therapeutic agent for NIDDM may have one or more of the following effects: a decrease in the acetyl-CoA/CoA ratio; decreased intermediates or products of fatty acid oxidation; increased intermediates or products of the Embden-Meyerhoff pathway, including lactic acid or lactate; increased intermediates and products of fatty acid synthesis; increased glycogen stores, and increased insulin sensitivity. Conversely, a modulator intended to effect preferential metabolism of fats (for example, in the treatment of obesity) may have one or more of the following effects: an increase in the acetyl-CoA/CoA ratio; increased intermediates or products of fatty acid oxidation; decreased intermediates or products of the Embden-Meyerhoff pathway, including lactic acid or lactate; decreased intermediates and products of fatty acid synthesis; decreased glycogen stores, and decreased insulin sensitivity. The foregoing in vivo assays may be performed in a cell in the context of a cell culture, a tissue explant, and/or an organism. Equivalent in vitro systems that duplicate one or more of the recited pathways may also be used to assay the modulator for desired activity.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Example 1

### Protein Expression and Purification

Residues 30-626 of mouse carnitine acetyltransferase (CRAT) was sub-cloned into the pET28a vector (Novagen) and over-expressed in E. coli at 20° C. The expression construct excluded the mitochondrial signal peptide (residues 1-29) of the native protein and introduced a hexa-histidine tag at the N-terminus. The soluble protein was purified by nickel-agarose affinity chromatography, anion exchange and gel-filtration chromatography. The protein was concentrated to 40 mg/ml in a buffer containing 20 mM Tris (pH 8.5), 200 mM NaCl, and 10 mM DTT. The N-terminal His-tag was not removed for crystallization.

For the production of selenomethionyl proteins, the expression construct was transformed into DL41(DE3) cells. The bacterial growth was carried out in defined LeMaster media (Hendrickson et al. (1990) Selenomethionyl proteins produced for analysis by multiwavelength anomalous diffraction (MAD): a vehicle for direct determination of three-dimensional structure, EMBO J 9:1665-1672), and the protein was purified using the same protocol as for the wild-type protein.

### Example 2

### Protein Crystallization

Crystals of mouse CRAT free enzyme were obtained at 4° C. by the sitting-drop vapor diffusion method. The reservoir solution contained 100 mM Tris (pH 7.5) and 20% (w/v) PEG3350. The protein was at 20 mg/ml concentration (diluted 1:1 with water from the stock). The crystals were cryo-protected with the introduction of 25% (v/v) PEG200 and flash frozen in liquid nitrogen.

Crystals of mouse CRAT in complex with carnitine were obtained at 4° C. by the sitting-drop vapor diffusion method. The reservoir solution contained 100 mM Tris (pH 8.0) and 12% (w/v) PEG3350. The protein was at 16 mg/ml concentration, and carnitine was present at 0.6 mM concentration.

For the CoA complex, crystals of the free enzyme of CRAT were soaked with 1.5 mM of acetyl-CoA overnight at 4° C. before they are treated with PEG200 and flash-frozen for data collection.

### Example 3

### Data Collection and Processing

X-ray diffraction data were collected on an ADSC CCD at the X4A beamline of Brookhaven National Laboratory. A seleno-methionyl single-wavelength anomalous diffraction (SAD) data set to 1.8 Å resolution was collected at 100K on the free enzyme crystal, and native reflection data sets were collected for the carnitine and CoA complexes. The diffraction images were processed and scaled with the HKL package (Otwinowski and Minor (1997) Processing of X-ray diffraction data collected in oscillation mode, Method Enzymol 276:307-326). The crystals belong to the space group C2, with cell dimensions of a=158.9 Å, b=89.6 Å, c=19.4 Å, and β=127.5° for the free enzyme crystal, a=160.7 Å, b=91.7 Å, c=122.6 Å, and β=128.8° for the carnitine complex, and a=162.3 Å, b=92.0 Å, c=122.9 Å, and β=129.0° for the CoA complex. There are two molecules in the crystallographic asymmetric unit. The data processing statistics are summarized in Table 1.

### Example 4

### Structure Determination and Refinement

The locations of 40 Se atoms were determined with the program SnB (Weeks and Miller (1999) The design and implementation of SnB v2.0, J Appl Cryst 32:120-124) and further confirmed with SHELXS (Sheldrick (1990) Acta Chest A46:467-473). Reflection phases to 1.8 Å resolution were calculated based on the SAD data and improved with the program SOLVE. (Terwilliger and Berendzen (1999) Automated structure solution for MIR and MAD, Acta Cryst D55:849-861), which also automatically located most of the residues in both molecules. The atomic model was fit into the electron density with the program O (Jones et al. (1991) Improved methods for building protein models in electron density maps and the location of errors in these models, Acta Cryst A47:110-119). The structure of the carnitine complex was determined by molecular replacement with the program COMO (Jogl et al. (2001) COMO: A program for combined molecular replacement, Acta Cryst D57: 1127-1134). The structure refinement was carried out with the program CNS (Brunger et al. (1998) Crystallography and NMR System: A new software suite for macromolecular structure determination, Acta Cryst D54:905-921). The statistics on the structure refinement are summarized in Table 1.

TABLE 1
Summary of crystallographic information

| Substrate | — | Carnitine | CoA |
|---|---|---|---|
| Maximum resolution (Å) | 1.8 | 1.9 | 2.3 |
| Number of observations | 749,015 | 347,562 | 135,285 |
| R_{merge} (%)¹ | 5.6 | 5.7 | 8.8 |
| Resolution range used for refinement | 30-1.8 Å | 20-1.9 Å | 30-2.3 Å |
| Number of reflections² | 235,313 | 105,409 | 53,398 |
| Completeness (%) (2σ cutoff) | 97 | 97 | 85 |
| R factor (%)³ | 18.8 | 20.1 | 27.0 |
| Free R factor (%) | 21.7 | 24.1 | 36.3 |
| rms deviation in bond lengths (Å) | 0.005 | 0.006 | 0.009 |
| rms deviation in bond angles (°) | 1.2 | 1.2 | 1.3 |

$1. {R}_{merge} = {\sum }_{h} {\sum }_{i} \vert {I}_{hi} - 〈 {I}_{h} 〉 \vert / {\sum }_{h} {\sum }_{i} {I}_{hi} .$
2. The number for the free enzyme includes both Friedel pairs.
$3. R = {\sum }_{h} \vert {F}_{h}^{o} - {F}_{h}^{c} \vert / {\sum }_{h} {F}_{h}^{o} .$

What is claimed is:

1. Atomic coordinates for murine carnitine acetyltransferase, as set forth in FIG. 7, or coordinates having a root mean square deviation (RMSD) therefrom, with respect to at least 50% of Cα atoms, of not more than 4.0 Å, in computer readable form.

2. A database containing the atomic coordinates of claim 1.

3. A computer displaying the atomic coordinates of claim 1.

4. Atomic coordinates for human carnitine acetyltransferase, as set forth in FIG. 10, or coordinates having a RMSD therefrom, with respect to at least 50% of Cα atoms, of not more than 4.0 Å, in computer readable form.

5. A database containing the atomic coordinates of claim 4.

6. A computer displaying the atomic coordinates of claim 4.

7. Atomic coordinates for murine carnitine acetyltransferase bound to carnitine, as set forth in FIG. 8, or coordinates having a RMSD therefrom, with respect to at least 50% of Cα atoms, of not more than 4.0 Å, in computer readable form.

8. A database containing the atomic coordinates of claim 7.

9. A computer displaying the atomic coordinates of claim 7.

10. Atomic coordinates for murine carnitine acetyltransferase bound to coenzyme A, as set forth in FIG. 9, or coordinates having a RMSD therefrom, with respect to at least 50% of Cα atoms, of not more than 4.0 Å, in computer readable form.

11. A database containing the atomic coordinates of claim 10.

12. A computer displaying the atomic coordinates of claim 10.

13. A virtual model of the reactive site of murine carnitine acetyltransferase.

14. A computer displaying the virtual model of the reactive site of murine carnitine acetyltransferase.

15. The virtual model of claim 13, comprising the atomic coordinates of atoms in amino acid residues Arg518, Thr465, Trp102, Tyr107, Glu347, His343, Phe566, Val569, Ser552, Ser454, Tyr452, Lys419, Lys423, Asp430 and Glu453 of murine carnitine acetyltransferase.

16. The computer of claim 14, wherein the virtual model comprises the atomic coordinates of atoms in amino acid residues Arg518, Thr465, Trp102, Tyr107, Glu347, His343, Phe566, Val569, Ser552, Ser454, Tyr452, Lys419, Lys423, Asp430 and Glu453 of murine carnitine acetyltransferase.

17. A virtual model of the reactive site of human carnitine acetyltransferase.

18. A computer displaying the virtual model of the reactive site of human carnitine acetyltransferase.

19. The virtual model of claim 17, comprising the atomic coordinates of atoms in amino acid residues Arg516, Thr463, Trp100, Tyr105, Glu345, His341, Phe564, Val567, Ser550, Ser452, Tyr450, Lys417, Lys421, Asp428 and Glu451 of human carnitine acetyltransferase.

20. The computer of claim 18, wherein the virtual model comprises the atomic coordinates of atoms in amino acid residues Arg516, Thr463, Trp100, Tyr105, Glu345, His341, Phe564, Val567, Ser550, Ser452, Tyr450, Lys417, Lys421, Asp428 and Glu451.

21. A method for rationally designing a modulator of a carnitine acyltransferase, comprising, the steps of (i) producing a computer readable model of a molecule comprising a carnitine acyltransferase reactive site; and (ii) using the model to design a test compound having a structure and a charge distribution compatable with the reactive site, wherein the test compound comprises a functional group that may interact with the reactive site to modulate carnitine acyltransferase activity. They wanted to know whether we could claim a method for carnitine acetyltransferase for CPT activity.

22. The method of claim 21, wherein the molecule comprising a carnitine acyltransferase active site has atomic coordinates as set forth in FIG. 7, or coordinates having a RMSD therefrom, with respect to at least 50% of Cα atoms, of not more than 4.0 Å.

23. The method of claim 21, wherein the molecule comprising a carnitine acyltransferase active site has atomic coordinates as set forth in FIG. 10, or coordinates having a RMSD therefrom, with respect to at least 50% of Cα atoms, of not more than 4.0 Å.

24. The method of claim 21, wherein the molecule comprises atoms of amino acid residues Arg518, Thr465, Trp102, Tyr107, Glu347, His343, Phe566, Val569, Ser552, Ser454, Tyr452, Lys419, Lys423, Asp430 and Glu453 having atomic coordinates as set forth in FIG. 7, or coordinates having a RMSD therefrom, with respect to at least 50% of Ca atoms, of not more than 4.0 Å.

25. The method of claim 21, wherein the molecule comprises atoms of amino acid residues Arg516, Thr463, Trp100, Tyr105, Glu345, His341, Phe564, Val567, Ser550, Ser452, Tyr450, Lys417, Lys421, Asp428 and Glu451 having atomic coordinates as set forth in FIG. 10, or coordinates having a RMSD therefrom, with respect to at least 50% of Cα atoms, of not more than 4.0 Å,

26. A method of screening a plurality of test compounds, as represented in computer readable form, to identify a modulator of a carnitine acyltransferase, comprising the steps of (i) producing a computer readable model of a molecule comprising a carnitine acyltransferase reactive site; and (ii) using the model to identify, from among the test compounds, a modulator compound having a structure and a charge distribution compatable with the reactive site and comprising a functional group that may interact with the reactive site to modulate carnitine acyltransferase activity.

27. The method of claim 26, wherein the molecule comprising a carnitine acyltransferase active site has atomic coordinates as set forth in FIG. 7, or coordinates having a RMSD therefrom, with respect to at least 50% of Cα atoms, of not more than 4.0 Å.

28. The method of claim 26, wherein the molecule comprising a carnitine acyltransferase active site has atomic coordinates as set forth in FIG. 10, or coordinates having a RMSD therefrom, with respect to at least 50% of Cα atoms, of not more than 4.0 Å.

29. The method of claim 26, wherein the molecule comprises atoms of amino acid residues Arg518, Thr465, Trp102, Tyr107, Glu347, His343, Phe566, Val569, Ser552, Ser454, Tyr452, Lys419, Lys423, Asp430 and Glu453 having atomic coordinates as set forth in FIG. 7, or coordinates having a RMSD therefrom, with respect to at least 50% of Cα atoms, of not more than 4.0 Å.

30. The method of claim 26, wherein the molecule comprises atoms of amino acid residues Arg516, Thr463, Trp100, Tyr105, Glu345, His341, Phe564, Val567, Ser550, Ser452, Tyr450, Lys417, Lys421, Asp428 and Glu451 having atomic coordinates as set forth in FIG. 10, or coordinates having a RMSD therefrom, with respect to at least 50% of Cα atoms, of not more than 4.0 Å.

31. A method for rationally designing a compound for the treatment of diabetes, comprising the steps of (i) producing a computer readable model of a molecule comprising a carnitine acyltransferase reactive site; and (ii) using the model to design a test compound having a structure and a charge distribution compatable with the reactive site, wherein the test compound comprises a functional group that may interact with the reactive site to modulate carnitine acyltransferase activity; and (iii) evaluating the effects of the test compound in vivo, wherein an effect selected from the group consisting of a decrease in the acetyl-CoA/CoA ratio; a decrease in an intermediate or product of fatty acid oxidation; an increase in an intermediate or product of the Embden-Meyerhoff pathway; an increase in an intermediate or product of fatty acid synthesis; an increase in glycogen; and increased insulin sensitivity is an indicator of effectiveness of the test compound.

32. The method of claim 31, wherein the molecule comprising a carnitine acyltransferase active site has atomic coordinates as set forth in FIG. 7, or coordinates having a RMSD therefrom, with respect to at least 50% of Cα atoms, of not more than 4.0 Å.

33. The method of claim 31, wherein the molecule comprising a carnitine acyltransferase active site has atomic coordinates as set forth in FIG. 10, or coordinates having a RMSD therefrom, with respect to at least 50% of Cα atoms, of not more than 4.0 Å.

34. The method of claim 31, wherein the molecule comprises atoms of amino acid residues Arg518, Thr465, Trp102, Tyr107, Glu347, His343, Phe566, Val569, Ser552, Ser454, Tyr452, Lys419, Lys423, Asp430 and Glu453 having atomic coordinates as set forth in FIG. 7, or coordinates having a RMSD therefrom, with respect to at least 50% of Cα atoms, of not more than 4.0 Å.

35. The method of claim 31, wherein the molecule comprises atoms of amino acid residues Arg516, Thr463, Trp100, Tyr105, Glu345, His341, Phe564, Val567, Ser550, Ser452, Tyr450, Lys417, Lys421, Asp428 and Glu451 having atomic coordinates as set forth in FIGURE, or coordinates having a RMSD therefrom, with respect to at least 50% of Cα atoms, of not more than 4.0 Å.

36. A method of screening a plurality of test compounds, as represented in computer readable form, to identify a compound useful for the treatment of diabetes, comprising the steps of (i) producing a computer readable model of a molecule comprising a carnitine acyltransferase reactive site; (ii) using the model to identify, from among the test compounds, a modulator compound having a structure and a charge distribution compatable with the reactive site and comprising a functional group that may interact with the reactive site to modulate carnitine acyltransferase activity; and (iii) evaluating the effects of the modulator compound in vivo, wherein an effect selected from the group consisting of a decrease in the acetyl-CoA/CoA ratio; a decrease in an intermediate or product of fatty acid oxidation; an increase in an intermediate or product of the Embden-Meyerhoff pathway; an increase in an intermediate or product of fatty acid synthesis; an increase in glycogen; and increased insulin sensitivity is an indicator of effectiveness of the modulator compound.

37. The method of claim 36, wherein the molecule comprising a carnitine acyltransferase active site has atomic coordinates as set forth in FIG. 7 with a root mean square deviation of from about 0 to 4 Å.

38. The method of claim 36, wherein the molecule comprising a carnitine acyltransferase active site has atomic coordinates as set forth in FIG. 10, or coordinates having a RMSD therefrom, with respect to at least 50% of Cα atoms, of not more than 4.0 Å.

39. The method of claim 36, wherein the molecule comprises atoms of amino acid residues Arg518, Thr465, Trp102, Tyr107, Glu347, His343, Phe566, Val569, Ser552, Ser454, Tyr452, Lys419, Lys423, Asp430 and Glu453 having atomic coordinates as set forth in FIG. 7, or coordinates having a RMSD therefrom, with respect to at least 50% of Cα atoms, of not more than 4.0 Å.

40. The method of claim 37, wherein the molecule comprises atoms of amino acid residues Arg516, Thr463, Trp100, Tyr105, Glu345, His341, Phe564, Val567, Ser550, Ser452, Tyr450, Lys417, Lys421, Asp428 and Glu451 having atomic coordinates as set forth in FIG. 10, or coordinates having a RMSD therefrom, with respect to at least 50% of Cα atoms, of not more than 4.0 Å.

41. A set of atomic coordinates, as set forth in FIG. 7, or with coordinates having a RMSD therefrom, with respect to at least 50% of Cα atoms, of not more than 2.0 Å, wherein said coordinates define a three dimensional structure of crystalline mammalian CRAT.

42. A set of atomic coordinates, as set forth in FIG. 10, or with coordinates having a RMSD therefrom, with respect to at least 50% of Cα atoms, of not more than 2.0 Å, wherein said coordinates define a three dimensional structure of crystalline mammalian CRAT.

43. A crystalline form of mammalian CRAT, wherein the crystalline form of the mammalian CRAT is capable of being used for X-ray crystallographic studies, and wherein the crystalline form of the mammalian CRAT has a crystal structure with atomic structural coordinates as set forth in FIG. 7, or with coordinates having a RMSD therefrom, with respect to at least 50% of Cα atoms, of not more than 4.0 Å.

44. The crystalline form of mammalian CRAT of claim 43 further comprising carnitine.

45. The crystalline form of mammalian CRAT of claim 43 further comprising CoA.


SEQUENCE LISTING (3 sequences)

SEQ ID NO: 1 - protein, 626 aa, Mus musculus
MLAFAARTVVKPLGLLKPSSLMKVSGRFKAHQDALPRLPVPPLQQSLDYYLKALQPIVSEEEWAHTKQLVDEFQTSGGVGERLQKGLERRAKKMENWLSEWWLKTAYLQFRQPVVIYSSPGVILPKQDFVDLQGQLRFAAKLIEGVLDFKSMIDNETLPVEFLGGQPLCMNQYYQILSSCREVGPKQDSVVNFLKSKRPPTHITVVHNYQFFELDVYNHDGTPLTSDQIFVQLEKIWNSSLQSNKEPVGILTSNHRNTWAKAYNNLIKDKVNRESVNSIQKSIFTVCLDKQVPRVSDDVYRNHVAGQMLHGGGSKFNSGNRWFDKTLQFIVAEDGSCGMVYEHAAAEGPPIVALVDHVMEYTKKPELVRSPMVPLPMPKKLRFNITPEIKNDIEKAKQNLSIMIQDLDIMMLTFHHFGKDFPKSEKLSPDAFIQVALQLAYYRIYGQACATYESASLRMFHLGRTDTIRSASIDSLAFVKGMGDSTVPEQQKVELLRKAVQAHRAYTDRAIRGEAFDRHLLGLKLQAIEDLVSMPDIFMDTSYAIAMHFNLSTSQVPAKTDCVMFFGPVVPDGYGICYNPMEAHINLFVSAYNSCAETNAARMAHYLEKALLDMRTLLQNHPRAKL

SEQ ID NO: 2 - protein, 633 aa, Homo sapiens
GKMSRATKIWMGMVKIFSGRKPMLYSFQTSLPRLPVPAVKDTVNRYLQSVRPLMKEEDFKRMTALAQDFAVGLGPRLQWYLKLKSWWATNYVSDWWEEYIYLRGRGPLMVNSNYYAMDLLYILPTHIQAARAGNAIHAILLYRRKLDREEIKPIRLLGSTIPLCSAQWERMFNTSRIPGEETDTIQHMRDSKHIVVYHRGRYFKVWLYHDGRLLKPREMEQQMQRILDNTSEPQPGEARLAALTAGDRVPWARCRQAYFGRGKNKQSLDAVEKAAFFVTLDETEEGYRSEDPDTSMDSYAKSLLHGRCYDRWFDKSFTFVVFKNGKMGLNAEHSWADAPIVAHLWEYVMSIDSLQLGYAEDGHCKGDINPNIPYPTRLQWDIPGECQEVIETSLNTANLLANDVDFHSFPFVAFGKGIIKKCRTSPDAFVQLALQLAHYKDMGKFCLTYEASMTRLFREGRTETVRSCTTESCDFVRAMVDPAQTVEQRLKLFKLASEKHQHMYRLAMTGSGIDRHLFCLYVVSKYLAVESPFLKEVLSEPWRLSTSQTPQQQVELFDLENNPEYVSSGGGFGPVADDGYGVSYILVGENLINFHISSKFSCPETDSHRFGRHLKEAMTDIITLFGLSSNSKK

SEQ ID NO: 3 - protein, 630 aa, Homo sapiens
GKTSNLTRIWAMCIRLLSSRHPMLYSFQTSLPKLPVPRVSATIQRYLESVRPLLDDEEYYRMELLAKEFQDKTAPRLQKYLVLKSWWASNYVSDWWEEYIYLRGRSPLMVNSNYYVMDLVLIKNTDVQAARLGNIIHAMIMYRRKLDREEIKPVMALGIVPMCSYQMERMFNTTRIPGKDTDVLQHLSDSRHVAVYHKGRFFKLWLYEGARLLKPQDLEMQFQRILDDPSPPQPGEEKLAALTAGGRVEWAQARQAFFSSGKNKAALEAIERAAFFVALDEESYSYDPEDEASLSLYGKALLHGNCYNRWFDKSFTLISFKNGQLGLNAEHAWADAPIIGHLWEFVLGTDSFHLGYTETGHCLGKPNPALAPPTRLQWDIPKQCQAVIESSYQVAKALADDVELYCFQFLPFGKGLIKKCRTSPDAFVQIALQLAHFRDRGKFCLTYEASMTRMFREGRTETVRSCTSESTAFVQAMMEGSHTKADLRDLFQKAAKKHQNMYRLAMTGAGIDRHLFCLYLVSKYLGVSSPFLAEVLSEPWRLSTSQIPQSQIRMFDPEQHPNHLGAGGGFGPVADDGYGVSYMIAGENTIFFHISSKFSSSETNAQRFGNHIRKALLDIADLFQVPKAYS